(19) United States
(12) Patent Application Publication
(10) Pub. No.: US 2023/0310428 A1
(43) Pub. Date: Oct. 5, 2023

(54) EGFR INHIBITOR AND PREPARATION METHOD AND USE THEREOF

(71) Applicant: HONGYUN BIOTECH CO., LTD., NANJING (CN)

(72) Inventors: Xianming DENG, XIAMEN (CN); Wei HUANG, NANJING (CN); Zhenhua WU, XIAMEN (CN); Yachuang WU, BEIJING (CN); Caihong YUN, BEIJING (CN); Jianming ZHANG, SHANGHAI (CN); Xin HUANG, NANJING (CN)

(73) Assignee: HONGYUN BIOTECH CO., LTD., NANJING (CN)

(21) Appl. No.: 17/999,779

(22) PCT Filed: May 24, 2021
(86) PCT No.: PCT/CN2021/095366

§ 371 Date: Nov. 23, 2022

(30) Foreign Application Priority Data
May 25, 2020 (CN) 202010450437.0
Aug. 17, 2020 (CN) 202010825263.1
May 13, 2021 (CN) 202110524157.4

(51) Int. Cl. A61K 31/506; A61K 39/395; C07D 401/14; C07D 403/12; C07D 403/14; A61K 31/55; C07D 495/04; A61K 31/519; C07D 491/048; C07D 487/04

(52) U.S. Cl. CPC A61K 31/506; A61K 31/55; A61K 31/519; A61K 39/3955; C07D 401/14; C07D 403/12; C07D 403/14; C07D 487/04; C07D 491/048; C07D 495/04

(57) ABSTRACT

A compound represented by the following general formula, a preparation method thereof, a pharmaceutical composition containing the compound, and a use of the compound in preparation of drugs for preventing and/or treating EGFR kinase-mediated cancers and other diseases.

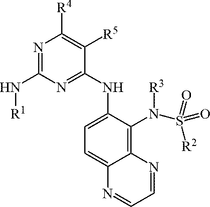

### TECHNICAL FIELD

The present invention relates to the field of medicinal chemistry, and in particular to an EGFR inhibitor, and its preparation method and use.

### BACKGROUND ART

Cancer is one of the most major diseases threatening human life and health at present. In recent years, about 18 million new cancer cases arise worldwide each year, and about 9.5 million deaths are due to the cancer. Fortunately, with the develop of targeted drugs, some progresses have been achieved in the field of cancer therapy. For instance, as for non-small cell lung cancer, a small molecular inhibitor targeting epidermal growth factor receptor (EGFR) can selectively hinder the binding of EGFR to ATP, so as to inhibit the activation and downstream signaling pathways of EGFR, which leads to stagnations on the growth, proliferation and transfer of cells, and further to the apoptosis. Thereby, effective controls of EGFR-mediated tumors are achieved.

Unfortunately, acquired resistance is easy to occur after clinical use of the EGFR inhibitors. The main way is the point mutation of the EGFR (for example, the most common T790M mutation) which leads to the incapacity of small molecules to effectively bind to the EGFR and the occurrence of the case where the inhibiting effects decrease or even disappear. In response to this situation, researchers have developed a new generation of inhibitors step by step, which can effectively overcome the drug resistance problem of the previous generation. At present, the third-generation EGFR inhibitors, Oxitinib and Almonertinib, have been approved in China, which can effectively overcome the T790M mutation, and have weak activities against wild type EGFR, reducing the toxicity and side effects of second-generation drugs. However, the drug resistance to Oxitinib has emerged (Nature Medicine, 2015, 21, 560-562). The main mechanism is that the Cysteine residue at 797 position of EGFR mutates to a Serine residue (C797S), and this mutation causes the disappearance of covalent actions between the Cys and the inhibitor so that the drug resistance emerges, accounting for about 40%.

Hence, the development of fourth-generation EGFR inhibitors which can overcome the C797S mutation will have great significance and clinical values.

### SUMMARY OF INVENTION

After extensive and in-depth research, the inventors of the present invention have designed and synthesized a series of small molecule compounds having novel structures, which have good inhibitory effects on a plurality of common EGFR kinase mutants in clinical, including mutants comprising the C797S mutation.

The present invention provides a compound represented by the general formula:

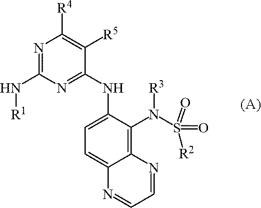

or a stereoisomer, a prodrug, a pharmaceutically acceptable salt or a pharmaceutically acceptable solvate of the above compound.

The definitions of substituents and symbols in the formula are described in detail below.

One object of the present invention is to provide a compound having EGFR kinase inhibitory activity, and a stereoisomer thereof, a prodrug thereof, a pharmaceutically acceptable salt thereof or a pharmaceutically acceptable solvate thereof.

Another object of the present invention is to provide a method of preparing the above compound.

Another object of the present invention is to provide a pharmaceutical composition comprising the above compound.

Another object of the present invention is to provide use of the above compound and the pharmaceutical composition comprising the above compound in the manufacture of a medicament for preventing and/or treating EGFR kinase-mediated cancers or other diseases.

Another object of the present invention is to provide a method of treating cancers, the method comprises administrating an effective amount of the compound or composition of the present invention to a subject.

### SPECIFIC MODES FOR CARRYING OUT THE INVENTION

Various specific embodiments, modes and examples are described herein, including exemplary embodiments and definitions, to understand the claimed invention. While the following detailed description sets forth specific preferred embodiments, those skilled in the art will appreciate that these embodiments are illustrative only, and that the present invention can be practiced in other ways. For the purpose of determining infringement, the scope of the present invention will cover any one or more of the appended claims, including equivalents thereof, and elements or limitations equivalent to those recited.

The present invention is achieved by the following technical solutions.

In the first aspect of the present invention, the present invention provides a compound having the general formula (A):

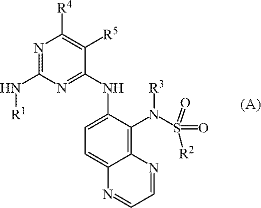

or a stereoisomer, a prodrug, a pharmaceutically acceptable salt or a pharmaceutically acceptable solvate thereof, wherein:
R¹ is selected from:
   1) H, C₁-C₆ alkyl, C₁-C₆ fluorine-containing alkyl, C₃-C₈ cycloalkyl, C₁-C₆ heteroatom-containing alkyl, C₃-C₈ heteroatom-containing cycloalkyl;
   2) 2-N,N-dimethylaminoethyl, 3-N,N-dimethylaminopropyl, 2-N,N-diethylaminoethyl, 3-N,N-diethylaminopropyl, 2-(N-methylpiperazinyl)ethyl, 2-(N-acetylpiperazinyl)ethyl, 2-morpholinylethyl, 2-thiomorpholinylethyl, 2-piperidylethyl, 2-hydroxyethyl, 3-hydroxypropyl, 2-methoxyethyl, N-methyl-4-piperidyl, N-ethyl-4-piperidyl, N-propyl-4-piperidyl, N-isopropyl-4-piperidyl, N-hydroxyethyl-4-piperidyl, N-cyanomethyl-4-piperidyl, N-(2-cyanoethyl)-4-piperidyl, N-acetyl-4-piperidyl, N-propionyl-4-piperidyl, N-isopropionyl-4-piperidyl, N-cyclopropylformyl-4-piperidyl, N-methylsulfonyl-4-piperidyl, N-ethylsulfonyl-4-piperidyl, N-propylsulfonyl-4-piperidyl, N-isopropylsulfonyl-4-piperidyl, N-cyclopropylsulfonyl-4-piperidyl, 3-N,N-dimethylaminocyclopentyl, 3-N,N-diethylaminocyclopentyl, 3-N,N-dipropylaminocyclopentyl, 3-N,N-isopropylaminocyclopentyl, 4-aminocyclohexyl, 4-N,N-dimethylaminocyclohexyl, 4-N,N-diethylaminocyclohexyl, 4-N,N-dipropylaminocyclohexyl, 4-N,N-diisopropylaminocyclohexyl, 4-acetylaminocyclohexyl, 4-propionylaminocyclohexyl, 4-isopropylformylaminocyclohexyl, 4-methylsulfonylaminocyclohexyl, 4-ethylsulfonylaminocyclohexyl, 4-propylsulfonylaminocyclohexyl, 4-isopropylsulfonylaminocyclohexyl, 4-cyclopropylsulfonylaminocyclohexyl, 4-hydroxycyclohexyl, 4-methoxycyclohexyl, 4-ethoxycyclohexyl, 4-isopropoxycyclohexyl;
   3) 1-methyl-3-pyrrolyl, 1-ethyl-3-pyrrolyl, 1-isopropyl-3-pyrrolyl, 1-cyclopropyl-3-pyrrolyl, 1-methyl-3-pyrazolyl, 1-ethyl-3-pyrazolyl, 1-isopropyl-3-pyrazolyl, 1-cyclopropyl-3-pyrazolyl, 1-hydroxyethyl-3-pyrazolyl, 1,5-dimethyl-3-pyrazolyl, 1,5-diethyl-3-pyrazolyl, 1,5-diisopropyl-3-pyrazolyl, 1-methyl-4-pyrazolyl, 1-ethyl-4-pyrazolyl, 1-isopropyl-4-pyrazolyl, 1-cyclopropyl-4-pyrazolyl, 1-hydroxyethyl-4-pyrazolyl, 1-(N-methyl-4-piperidyl)-4-pyrazolyl, 1-(N-t-butoxycarbonyl-4-piperidyl)-4-pyrazolyl, 1,3-dimethyl-4-pyrazolyl, 1-methyl-3-ethyl-4-pyrazolyl, 1-methyl-3-isopropyl-4-pyrazolyl, 1-ethyl-3-methyl-4-pyrazolyl, 1,3-diethyl-4-pyrazolyl, 1-ethyl-3-isopropyl-4-pyrazolyl, 1-isopropyl-3-methyl-4-pyrazolyl, 1-isopropyl-3-ethyl-4-pyrazolyl, 1,3-diisopropyl-4-pyrazolyl, 1,3-dimethyl-5-pyrazolyl, 1-methyl-4-imidazolyl, 3-methyl-5-isoxazolyl;
   4) 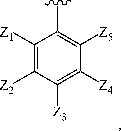
   wherein Z₁, Z₂, Z₃, Z₄, Z₅ each are independently selected from the following groups, and Z₁, Z₂, Z₃, Z₄, Z₅ are not hydrogen at the same time:
      (1) H, F, Cl, Br, I, nitro, cyano,
      (2) C₁-C₆ alkyl, C₁-C₆ alkoxy, C₁-C₆ oxygen-containing alkyl, C₁-C₆ fluorine-containing alkyl, C₁-C₆ fluorine-containing alkoxy, C₃-C₆ cycloalkyl, C₂-C₆ alkenyl,
      (3) 2-N,N-dimethylaminoethoxy, 3-N,N-dimethylaminopropoxy, 2-(N-methylpiperazinyl)ethoxy, 2-(N-acetylpiperazinyl)ethoxy, 2-morpholinylethoxy, 2-thiomorpholinylethoxy, 2-piperidylethoxy, 3-N,N-dimethylaminopropoxy, 3-N,N-diethylaminopropoxy, 3-N,N-diisopropylaminopropoxy, 3-(N-methylpiperazinyl)propoxy, 3-(N-acetylpiperazinyl)propoxy, 3-morpholinylpropoxy, 3-thiomorpholinylpropoxy, 3-piperidylpropoxy, 2-pyridylmethoxy, 3-pyridylmethoxy, 4-pyridylmethoxy, phenylmethoxy, monohalo-substituted phenylmethoxy, gem-dihalo-substituted phenylmethoxy, hetero-dihalo-substituted phenylmethoxy,
      (4) piperazinyl, N-methylpiperazinyl, N-ethylpiperazinyl, N-n-propylpiperazinyl, N-isopropylpiperazinyl, 4-(piperidyl-4-)piperazinyl, 4-(N-methylpiperidyl-4-)piperazinyl, 4-(N-ethylpiperidyl-4-)piperazinyl, 4-(N-n-propylpiperidyl-4-)piperazinyl, 4-(N-isopropylpiperidyl-4-)piperazinyl, 4-(azetidin-3-yl)piperazinyl, 4-(N-methyl-azetidin-3-yl)piperazinyl, 4-(N-ethyl-azetidin-3-yl)piperazinyl, 4-(N-n-propyl-azetidin-3-yl)piperazinyl, 4-(N-isopropyl-azetidin-3-yl)piperazinyl, N-cyclopropylpiperazinyl, N-(2-hydroxyethyl)piperazinyl, N-(2-N,N-dimethylaminoethyl)piperazinyl, N-(2-N,N-diethylaminoethyl)piperazinyl, N-(2-cyanoethyl)piperazinyl, N-cyanomethylpiperazinyl, N-(3-hydroxypropyl)piperazinyl, N-(3-N,N-dimethylaminopropyl)piperazinyl, N-(3-N,N-diethylaminopropyl)piperazinyl, N-(N-methyl-4-piperidyl)piperazinyl, N-(N-ethyl-4-piperidyl)piperazinyl, N-t-butoxycarbonylpiperazinyl, N-acetylpiperazinyl, N-propionylpiperazinyl, N-isobutyrylpiperazinyl, N-cyclopropylformylpiperazinyl, N-methylsulfonylpiperazinyl, N-ethylsulfonylpiperazinyl, N-n-propylsulfonylpiperazinyl, N-isopropylsulfonylpiperazinyl, N-cyclopropylsulfonylpiperazinyl, 2-oxo-piperazin-4-yl,
      (5) morpholinyl, 2,6-dimethylmorpholinyl, thiomorpholinyl,
      (6) tetrahydropyrrolyl, 3-(N,N-dimethylamino)tetrahydropyrrolyl, 3-(N,N-diethylamino)tetrahydropyrrolyl,
      (7) imidazolyl, 4-methyl-1-imidazolyl,
      (8) piperidyl, 4-N,N-dimethylaminopiperidyl, 4-N,N-diethylaminopiperidyl, 4-N,N-diisopropylaminopiperidyl, 4-hydroxypiperidyl, 4-cyanopiperidyl, 4-methoxypiperidyl, 4-ethoxypiperidyl, 4-(piperazinyl-1-)piperidyl, 4-(N-methylpiperazinyl-1-)piperidyl, 4-(N-ethylpiperazinyl-1-)piperidyl, 4-(N-isopropylpiperazinyl-1-)piperidyl, 4-(N-acetylpiperazinyl-1-)piperidyl, 4-(N-t-butoxycarbonylpiperazinyl-1-)piperidyl, 4-(N-methylsulfonylpiperazinyl-1-)piperidyl, 4-(N-(2-hydroxyethyl)piperazinyl-1-)piperidyl, 4-(N-(2-cyanoethyl)piperazinyl-1-)piperidyl, 4-(N-(3-hydroxypropyl)piperazinyl-1-)piperidyl, 4-(N-(2-N,N-dimethylaminoethyl)piperazinyl-1-)piperidyl, 4-(N-(2-N,N-diethylethyl)piperazinyl-1 -)piperidyl, 4-(N-(3-N,N-dimethylpropyl)piperazinyl-1-)piperidyl, 4-(N-(3-N,N-diethylpropyl)piperazinyl-1-)piperidyl, 4-(tetrahydropyrrolyl-1-)piperidyl, 4-(3-N,N-dimethylaminotetrahydropyrrolyl-1-)piperidyl, 4-(3-N,N-diethylaminotetrahydropyrrolyl-1-)piperidyl,
      (9) piperidin-4-yl, N-methyl-4-piperidyl, N-ethyl-4-piperidyl, N-isopropyl-4-piperidyl, N-acetyl-4-piperidyl, N-t-butoxycarbonyl-4-piperidyl, N-methylsulfonyl-4-piperidyl, N-(2-hydroxyethyl)-4-piperidyl, N-(2-N,N-dimethylaminoethyl)-4-piperidyl, N-(2-N,N-diethylaminoethyl)-4-piperidyl, N-(2-cyanoethyl)-4-piperidyl, N-(3-hydroxypropyl)-4-piperidyl, N-(3-N,N-dimethylaminopropyl)-4-piperidyl, N-(3-N,N-diethylaminopropyl)-4-piperidyl, N-(3-cyanopropyl)-4-piperidyl, N-cyanomethylene-4-piperidyl,
      (10) amino, N,N-dimethylamino, N,N-diethylamino, N,N-di-n-propylamino, N,N-diisopropylamino, 2-hydroxyethylamino, 2-N,N-dimethylaminoethylamino, 2-morpholinylethylamino, 2-(N-methylpiperazinyl)ethylamino, 3-N,N-dimethylaminopropylamino, 3-N,N-diethylaminopropylamino, 3-N,N-diisopropylam inopropylamino, 3-morpholinylpropylamino, 3-(N-methylpiperazinyl)propylamino, N-methylpiperidyl-4-amino, N-ethylpiperidyl-4-amino, N-n-propylpiperidyl-4-amino, N-isopropylpiperidyl-4-amino, acetamido, propionamido, 2-(N,N-dimethylamino)acetamido, 2-hydroxyacetamido, 2-methoxyacetamido, methylsulfamido, ethylsulfamido, n-propylsulfamido, isopropylsulfamido, cyclopropylsulfamido, N-methyl-N-(N-methyl-3-tetrahydropyrrolyl)amino, N-(N-methyl-3-tetrahydropyrrolyl)amino, N-methyl-N-(2-N,N-dimethylamino)ethylamino,
      (11) aminomethylene, N,N-dimethylaminomethylene, N,N-diethylaminomethylene, 4-methyl-1-piperazinylmethylene, 4-ethyl-1-piperazinylmethylene, 4-propyl-1-piperazinylmethylene, 4-isopropyl-1-piperazinylmethylene, 4-(2-hydroxyethyl)-1-piperazinylmethylene, 4-(2-N,N-dimethylaminoethyl)-1-piperazinylmethylene, piperidyl-1-methylene, 4-(N,N-dimethylamino)-1-piperidylmethylene, 4-hydroxypiperidyl-1-methylene, tetrahydropyrrolyl-1-methylene, 3-(N,N-dimethylamino)-1-tetrahydropyrrolylmethylene, benzyl, cyclopropylmethylene, cyclobutylmethylene, cyclopentylmethylene, cyclohexylmethylene,
      (12) aminoformyl, methylaminoformyl, N,N-dimethylaminoformyl, ethylaminoformyl, cyclopropylaminoformyl, cyclobutylaminoformyl, cyclopentylaminoformyl, cyclohexylaminoformyl, aminoacetyl, methylaminoacetyl, 2-(N,N-dimethylamino)acetyl, ethylam inoacetyl, cyclopropylaminoacetyl, cyclobutylaminoacetyl, cyclopentylaminoacetyl, cyclohexylaminoacetyl, tetrahydropyrrolyl-1-formyl, 3-(N,N-dimethylamino)tetrahydropyrrolyl-1-formyl, 3-(N,N-diethylamino)tetrahydropyrrolyl-1-formyl, morpholinyl-4-formyl, thiomorpholinyl-4-formyl, 2,6-dimethylmorpholinyl-4-formyl, piperidyl-1-formyl, 4-hydroxypiperidyl-1-formyl, 4-(N,N-dimethylamino)piperidyl-1-formyl, 4-(N,N-diethylamino)piperidyl-1-formyl, 4-(N-methyl-1-piperazinyl)piperidyl-1-formyl, 4-(N-ethyl-1-piperazinyl)piperidyl-1-formyl, N-methylpiperazinyl-1-formyl, N-ethylpiperazinyl-1-formyl, N-isopropylpiperazinyl-1-formyl, N-(2-hydroxyethyl)piperazinyl-1-formyl, N-(2-N,N-dimethylaminoethyl)piperazinyl-1-formyl, N-(2-N,N-diethylaminoethyl)piperazinyl-1-formyl, 4-(N-methyl-4-piperidyl)piperazinyl-1-formyl, 4-(N-ethyl-4-piperidyl)piperazinyl-1-formyl,
      (13) hydroxyformyl, methoxyformyl, ethoxyformyl, propoxyformyl, isopropoxyformyl, n-butoxyformyl, iso-butoxyformyl, t-butoxyformyl,
      (14) hydroxysulfonyl, aminosulfonyl, methylaminosulfonyl, ethylaminosulfonyl, propylaminosulfonyl, isopropylaminosulfonyl, cyclopropylaminosulfonyl, cyclobutylaminosulfonyl, cyclopentylaminosulfonyl, cyclohexylaminosulfonyl, tetrahydropyrrolyl-1-sulfonyl, 3-(N,N-dimethylamino)tetrahydropyrrolyl-1-sulfonyl, 3-(N,N-diethylamino)tetrahydropyrrolyl-1-sulfonyl, morpholinyl-4-sulfonyl, thiomorpholinyl-4-sulfonyl, 2,6-dimethylmorpholinyl-4-sulfonyl, piperidyl-1-sulfonyl, 4-hydroxypiperidyl-1-sulfonyl, 4-(N,N-dimethylamino)piperidyl-1-sulfonyl, 4-(N,N-diethylamino)piperidyl-1-sulfonyl, 4-(N-methyl-1-piperazinyl)piperidyl-1-sulfonyl, 4-(N-ethyl-1-piperazinyl)piperidyl-1-sulfonyl, N-methylpiperazinyl-1-sulfonyl, N-ethylpiperazinyl-1-sulfonyl, N-isopropylpiperazinyl-1-sulfonyl, N-(2-hydroxyethyl)piperazinyl-1-sulfonyl, N-(2-N,N-dimethylaminoethyl)piperazinyl-1-sulfonyl, N-(2-N,N-diethylaminoethyl)piperazinyl-1-sulfonyl, 4-(N-methyl-4-piperidyl)piperazinyl-1-sulfonyl, 4-(N-ethyl-4-piperidyl)piperazinyl-1-sulfonyl,
      (15) aminoformamido, methylaminoformamido, ethylaminoformamido, propylaminoformamido, isopropylaminoformamido, cyclopropylaminoformamido, cyclobutylaminoformamido, cyclopentylaminoformamido, piperidyl-1-formamido, 4-hydroxypiperidyl-1-formamido, 4-N,N-dimethylpiperidyl-1-formamido, 4-N,N-diethylpiperidyl-1-formamido, tetrahydropyrrolyl-1-formamido, 3-N,N-dimethyltetrahydropyrrolyl-1-formamido, 3-N,N-diethyltetrahydropyrrolyl-1-formamido, N-methylpiperazinyl-1-formamido, N-ethylpiperazinyl-1-formamido, N-acetylpiperazinyl-1-formamido, N-t-butoxycarbonylpiperazinyl-1-formamido, N-(2-hydroxyethyl)piperazinyl-1-formamido, N-(2-cyanoethyl)piperazinyl-1-formamido, N-(2-N,N-dimethylaminoethyl)piperazinyl-1-formamido, N-(2-N,N-diethylaminoethyl)piperazinyl-1-formamido, N-(3-hydroxypropyl)piperazinyl-1-formamido, N-(3-N,N-dimethylpropyl)piperazinyl-1-formamido, N-(3-N,N-diethylpropyl)piperazinyl-1-formamido, morpholinyl-1-formamido, 3,5-dimethylmorpholinyl-1-formamido, 4-(tetrahydropyrrolyl)piperidyl-1-formamido, 4-(N-methyl-1-piperazinyl)piperidyl-1-formamido, 4-(N-ethyl-1-piperazinyl)piperidyl-1-formamido, 4-(N-acetyl-1-piperazinyl)piperidyl-1-formamido, N-(N-methyl-4-piperidyl)piperazinyl-1-formamido,
      (16) Z₃ and Z₄ may form an oxygen-containing substituted or unsubstituted 5- to 7-membered ring; the substituent may be selected from the same substituents as described above for Z₁,
      (17) Z₃ and Z₄ may form a nitrogen-containing substituted or unsubstituted 5- to 7-membered ring; the substituent may be selected from the same substituents as described above for Z₁,
      (18) 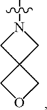 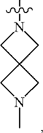 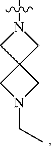 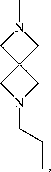 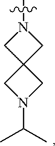 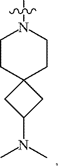 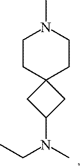 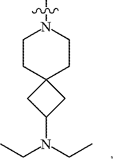 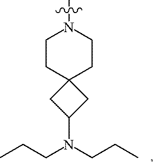 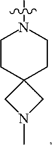 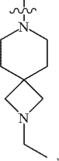 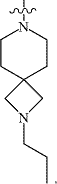 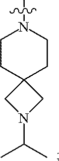
   5) 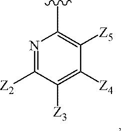
   wherein Z₂, Z₃, Z₄, Z₅ are the same as defined in 4), and Z₂, Z₃, Z₄, Z₅ are not hydrogen at the same time;
   6) 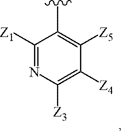
   wherein Z₁, Z₃, Z₄, Z₅ are the same as defined in 4), and Z₁, Z₃, Z₄, Z₅ are not hydrogen at the same time;
   7) 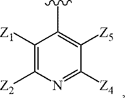
   wherein Z₁, Z₂, Z₄, Z₅ are the same as defined in 4), and Z₁, Z₂, Z₄, Z₅ may be hydrogen at the same time;
   8) 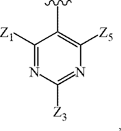
   wherein Z₁, Z₃, Z₅ are the same as defined in 4), and Z₁, Z₃, Z₅ are not hydrogen at the same time;
R², R³ each are independently selected from:
   H, C₁-C₆ alkyl, C₁-C₆ fluorine-containing alkyl, C₃-C₆ cycloalkyl, or C₃-C₆ fluorine-containing cycloalkyl;
R⁴, R⁵ each are independently selected from:
   H, F, Cl, Br, C₁-C₆ fluorine-containing alkyl (e.g., trifluoromethyl), cyano, nitro, C₁-C₆ alkyl (such as, methyl, ethyl or isopropyl), C₁-C₆ alkoxy (such as methoxy or ethoxy), or C₃-C₆ cycloalkyl (e.g., cyclopropyl),
   or, R⁴, R⁵ and carbon atoms linked thereto together form a N-, O- or S-containing 5-membered ring.

In some embodiments, R¹ is selected from:
1) H, C₁-C₆ alkyl, C₁-C₆ fluorine-containing alkyl, C₃-C₈ cycloalkyl, C₁-C₆ heteroatom-containing alkyl, C₃-C₈ heteroatom-containing cycloalkyl;
2) 2-N,N-dimethylaminoethyl, 3-N,N-dimethylaminopropyl, 2-N,N-diethylaminoethyl, 3-N,N-diethylaminopropyl, 2-(N-methylpiperazinyl)ethyl, 2-(N-acetylpiperazinyl)ethyl, 2-morpholinylethyl, 2-thiomorpholinylethyl, 2-piperidylethyl, 2-hydroxyethyl, 3-hydroxypropyl, 2-methoxyethyl, N-methyl-4-piperidyl, N-ethyl-4-piperidyl, N-propyl-4-piperidyl, N-isopropyl-4-piperidyl, N-hydroxyethyl-4-piperidyl, N-cyanomethyl-4-piperidyl, N-(2-cyanoethyl)-4-piperidyl, 3-N,N-dimethylaminocyclopentyl, 3-N,N-diethylaminocyclopentyl, 3-N,N-dipropylaminocyclopentyl, 3-N,N-isopropylaminocyclopentyl, 4-aminocyclohexyl, 4-N,N-dimethylaminocyclohexyl, 4-N,N-diethylaminocyclohexyl, 4-N,N-dipropylaminocyclohexyl, 4-N,N-diisopropylaminocyclohexyl, 4-hydroxycyclohexyl, 4-methoxycyclohexyl, 4-ethoxycyclohexyl, 4-isopropoxycyclohexyl;
3) 1-methyl-3-pyrrolyl, 1-ethyl-3-pyrrolyl, 1-isopropyl-3-pyrrolyl, 1-cyclopropyl-3-pyrrolyl, 1-methyl-3-pyrazolyl, 1-ethyl-3-pyrazolyl, 1-isopropyl-3-pyrazolyl, 1-cyclopropyl-3-pyrazolyl, 1-hydroxyethyl-3-pyrazolyl, 1,5-dimethyl-3-pyrazolyl, 1,5-diethyl-3-pyrazolyl, 1,5-diisopropyl-3-pyrazolyl, 1-methyl-4-pyrazolyl, 1-ethyl-4-pyrazolyl, 1-isopropyl-4-pyrazolyl, 1-cyclopropyl-4-pyrazolyl, 1-hydroxyethyl-4-pyrazolyl, 1,3-dimethyl-4-pyrazolyl, 1-methyl-3-ethyl-4-pyrazolyl, 1-methyl-3-isopropyl-4-pyrazolyl, 1-ethyl-3-methyl-4-pyrazolyl, 1,3-diethyl-4-pyrazolyl, 1-ethyl-3-isopropyl-4-pyrazolyl, 1-isopropyl-3-methyl-4-pyrazolyl, 1-isopropyl-3-ethyl-4-pyrazolyl, 1,3-diisopropyl-4-pyrazolyl, 1,3-dimethyl-5-pyrazolyl, 1-methyl-4-imidazolyl, 3-methyl-5-isoxazolyl;
4)

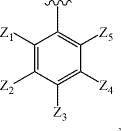

wherein Z₁, Z₂, Z₃, Z₄, Z₅ each are independently selected from the following groups, and Z₁, Z₂, Z₃, Z₄, Z₅ are not hydrogen at the same time:
   (1) H, F, Cl, Br, I, nitro, cyano,
   (2) C₁-C₆ alkyl, C₁-C₆ alkoxy, C₁-C₆ oxygen-containing alkyl, C₁-C₆ fluorine-containing alkyl, C₁-C₆ fluorine-containing alkoxy, C₃-C₆ cycloalkyl, C₂-C₆ alkenyl,
   (3) piperazinyl, N-methylpiperazinyl, N-ethylpiperazinyl, N-n-propylpiperazinyl, N-isopropylpiperazinyl, 4-(piperidyl-4-)piperazinyl, 4-(N-methylpiperidyl-4-)piperazinyl, 4-(N-ethylpiperidyl-4-)piperazinyl, 4-(N-n-propylpiperidyl-4-)piperazinyl, 4-(N-isopropylpiperidyl-4-)piperazinyl, 4-(azetidin-3-yl)piperazinyl, 4-(N-methyl-azetidin-3-yl)piperazinyl, 4-(N-ethyl-azetidin-3-yl)piperazinyl, 4-(N-n-propyl-azetidin-3-yl)piperazinyl, 4-(N-isopropyl-azetidin-3-yl)piperazinyl, N-cyclopropylpiperazinyl, N-(2-hydroxyethyl)piperazinyl, N-(2-N,N-dimethylaminoethyl)piperazinyl, N-(2-N,N-diethylaminoethyl)piperazinyl, N-(2-cyanoethyl)piperazinyl, N-cyanomethylpiperazinyl, N-(3-hydroxypropyl)piperazinyl, N-(3-N,N-dimethylaminopropyl)piperazinyl, N-(3-N,N-diethylaminopropyl)piperazinyl,
   (4) morpholinyl, 2,6-dimethylmorpholinyl, thiomorpholinyl,
   (5) tetrahydropyrrolyl, 3-(N,N-dimethylamino)tetrahydropyrrolyl, 3-(N,N-diethylamino)tetrahydropyrrolyl,
   (6) imidazolyl, 4-methyl-1-imidazolyl,
   (7) piperidyl, 4-N,N-dimethylaminopiperidyl, 4-N,N-diethylaminopiperidyl, 4-N,N-diisopropylaminopiperidyl, 4-hydroxypiperidyl, 4-cyanopiperidyl, 4-methoxypiperidyl, 4-ethoxypiperidyl, 4-(piperazinyl-1-)piperidyl, 4-(N-methylpiperazinyl-1-)piperidyl, 4-(N-ethylpiperazinyl-1-)piperidyl, 4-(N-isopropylpiperazinyl-1-)piperidyl, 4-(N-(2-hydroxyethyl)piperazinyl-1-)piperidyl, 4-(N-(2-cyanoethyl)piperazinyl-1-)piperidyl, 4-(N-(3-hydroxypropyl)piperazinyl-1-)piperidyl, 4-(N-(2-N,N-dimethylaminoethyl)piperazinyl-1-)piperidyl, 4-(N-(2-N,N-diethylethyl)piperazinyl-1 -)piperidyl, 4-(N-(3-N,N-dimethylpropyl)piperazinyl-1-)piperidyl, 4-(N-(3-N,N-diethylpropyl)piperazinyl-1-)piperidyl, 4-(tetrahydropyrrolyl-1-)piperidyl, 4-(3-N,N-dimethylaminotetrahydropyrrolyl-1-)piperidyl, 4-(3-N,N-diethylaminotetrahydropyrrolyl-1-)piperidyl,
   (8) piperidin-4-yl, N-methyl-4-piperidyl, N-ethyl-4-piperidyl, N-isopropyl-4-piperidyl, N-(2-hydroxyethyl)-4-piperidyl, N-(2-N,N-dimethylaminoethyl)-4-piperidyl, N-(2-N,N-diethylaminoethyl)-4-piperidyl, N-(2-cyanoethyl)-4-piperidyl, N-(3-hydroxypropyl)-4-piperidyl, N-(3-N,N-dimethylaminopropyl)-4-piperidyl, N-(3-N,N-diethylaminopropyl)-4-piperidyl, N-(3-cyanopropyl)-4-piperidyl, N-cyanomethylene-4-piperidyl,
   (9) amino, N,N-dimethylamino, N,N-diethylamino, N,N-di-n-propylamino, N,N-diisopropylamino, 2-hydroxyethylamino, 2-N,N-dimethylaminoethylamino, 3-N,N-dimethylaminopropylamino, 3-N,N-diethylaminopropylamino, 3-N,N-diisopropylaminopropylamino, N-methylpiperidyl-4-amino, N-ethylpiperidyl-4-amino, N-n-propylpiperidyl-4-amino, N-isopropylpiperidyl-4-amino, acetamido, propionamido, 2-(N,N-dimethylamino)acetamido, 2-hydroxyacetamido, 2-methoxyacetamido, methylsulfamido, ethylsulfamido, n-propylsulfamido, isopropylsulfamido, cyclopropylsulfamido, N-methyl-N-(N-methyl-3-tetrahydropyrrolyl)amino, N-(N-methyl-3-tetrahydropyrrolyl)amino, N-methyl-N-(2-N,N-dimethylamino)ethylamino,
   (10) 4-methyl-1-piperazinylmethylene, 4-ethyl-1-piperazinylmethylene, 4-propyl-1-piperazinylmethylene, 4-isopropyl-1-piperazinylmethylene, 4-(2-hydroxyethyl)-1-piperazinylmethylene, 4-(2-N,N-dimethylaminoethyl)-1-piperazinylmethylene, piperidyl-1-methylene, 4-(N,N-dimethylamino)-1-piperidylmethylene, 4-hydroxypiperidyl-1-methylene, tetrahydropyrrolyl-1-methylene, 3-(N,N-dimethylamino)-1-tetrahydropyrrolylmethylene, benzyl, cyclopropylmethylene, cyclobutylmethylene, cyclopentylmethylene, cyclohexylmethylene,
   (11) aminoformyl, methylaminoformyl, N,N-dimethylaminoformyl, ethylaminoformyl, aminoacetyl, methylaminoacetyl, 2-(N,N-dimethylamino)acetyl, ethylaminoacetyl, tetrahydropyrrolyl-1-formyl, 3-(N,N-dimethylamino)tetrahydropyrrolyl-1-formyl, 3-(N,N-diethylamino)tetrahydropyrrolyl-1-formyl, morpholinyl-4-formyl, thiomorpholinyl-4-formyl, 2,6-dimethylmorpholinyl-4-formyl, piperidyl-1-formyl, 4-hydroxypiperidyl-1-formyl, 4-(N,N-dimethylamino)piperidyl-1-formyl, 4-(N,N-diethylamino)piperidyl-1-formyl, 4-(N-methyl-1-piperazinyl)piperidyl-1-formyl, 4-(N-ethyl-1-piperazinyl)piperidyl-1-formyl, N-methylpiperazinyl-1-formyl, N-ethylpiperazinyl-1-formyl, N-isopropylpiperazinyl-1-formyl, N-(2-hydroxyethyl)piperazinyl-1-formyl, N-(2-N,N-dimethylaminoethyl)piperazinyl-1-formyl, N-(2-N,N-diethylaminoethyl)piperazinyl-1-formyl, 4-(N-methyl-4-piperidyl)piperazinyl-1-formyl, 4-(N-ethyl-4-piperidyl)piperazinyl-1-formyl,
   (12) hydroxysulfonyl, aminosulfonyl, methylaminosulfonyl, ethylaminosulfonyl, propylaminosulfonyl, isopropylaminosulfonyl, cyclopropylaminosulfonyl, cyclobutylaminosulfonyl, cyclopentylaminosulfonyl, cyclohexylaminosulfonyl, tetrahydropyrrolyl-1-sulfonyl, 3-(N,N-dimethylamino)tetrahydropyrrolyl-1-sulfonyl, 3-(N,N-diethylamino)tetrahydropyrrolyl-1-sulfonyl, morpholinyl-4-sulfonyl, thiomorpholinyl-4-sulfonyl, 2,6-dimethylmorpholinyl-4-sulfonyl, piperidyl-1-sulfonyl, 4-hydroxypiperidyl-1-sulfonyl, 4-(N,N-dimethylamino)piperidyl-1-sulfonyl, 4-(N,N-diethylamino)piperidyl-1-sulfonyl, N-methylpiperazinyl-1-sulfonyl, N-ethylpiperazinyl-1-sulfonyl, N-isopropylpiperazinyl-1-sulfonyl, N-(2-hydroxyethyl)piperazinyl-1-sulfonyl, N-(2-N,N-dimethylaminoethyl)piperazinyl-1-sulfonyl, N-(2-N,N-diethylaminoethyl)piperazinyl-1-sulfonyl,
   (13) aminoformamido, methylaminoformamido, ethylaminoformamido, rolaminoformamido, isorolaminoformamido,
   (14) 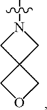 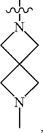 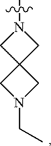 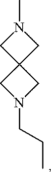 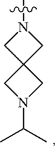 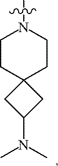 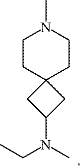 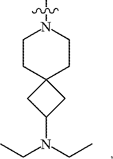 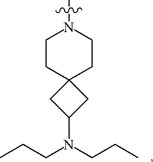 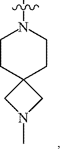 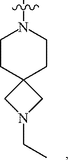 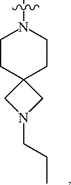 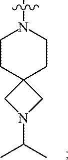
5)

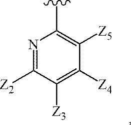

wherein Z₂, Z₃, Z₄, Z₅ are the same as defined in 4), and Z₂, Z₃, Z₄, Z₅ are not hydrogen at the same time;
6)

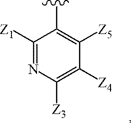

wherein Z₁, Z₃, Z₄, Z₅ are the same as defined in 4), and Z₁, Z₃, Z₄, Z₅ are not hydrogen at the same time;
7)

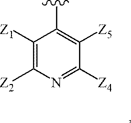

wherein Z₁, Z₂, Z₄, Z₅ are the same as defined in 4), and Z₁, Z₂, Z₄, Z₅ may be hydrogen at the same time;
8)

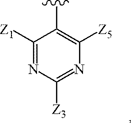

wherein Z₁, Z₃, Z₅ are the same as defined in 4), and Z₁, Z₃, Z₅ are not hydrogen at the same time;
9)

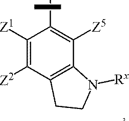

wherein Z¹, Z², Z⁵ are the same as defined in 4), and Z¹, Z², Z⁵ may be hydrogen at the same time, R^{x} is selected from the same substituents as described above for Z¹;
10)

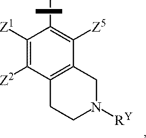

wherein Z₁, Z₂, Z₅ are the same as defined in 4), and Z₁, Z₂, Z₅ may be hydrogen at the same time, R^{y} is selected from the same substituents as described above for Z¹;
11)

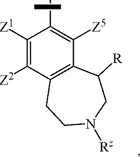

wherein Z₁, Z₂, Z₅ are the same as defined in 4), and Z₁, Z₂, Z₅ may be hydrogen at the same time, R^{z}, R each are independently selected from the same substituents as described above for Z₁.

In some embodiments, R¹ is selected from:
1) H, C₁-C₆ alkyl, C₃-C₈ cycloalkyl;
2) 2-N,N-dimethylaminoethyl, 3-N,N-dimethylaminopropyl, 2-N,N-diethylaminoethyl, 3-N,N-diethylaminopropyl, 2-hydroxyethyl, 3-hydroxypropyl, N-methyl-4-piperidyl, N-ethyl-4-piperidyl, N-propyl-4-piperidyl, N-isopropyl-4-piperidyl, 4-aminocyclohexyl, 4-N,N-dimethylaminocyclohexyl, 4-N,N-diethylaminocyclohexyl, 4-N,N-dipropylaminocyclohexyl, 4-N,N-diisopropylaminocyclohexyl;
3) 1-methyl-3-pyrazolyl, 1-ethyl-3-pyrazolyl, 1-isopropyl-3-pyrazolyl, 1-cyclopropyl-3-pyrazolyl, 1,5-dimethyl-3-pyrazolyl, 1,5-diethyl-3-pyrazolyl, 1,5-diisopropyl-3-pyrazolyl, 1-methyl-4-pyrazolyl, 1-ethyl-4-pyrazolyl, 1-isopropyl-4-pyrazolyl, 1-cyclopropyl-4-pyrazolyl, 1,3-dimethyl-4-pyrazolyl, 1-methyl-3-ethyl-4-pyrazolyl, 1-methyl-3-isopropyl-4-pyrazolyl, 1-ethyl-3-methyl-4-pyrazolyl, 1,3-diethyl-4-pyrazolyl, 1-ethyl-3-isopropyl-4-pyrazolyl, 1-isopropyl-3-methyl-4-pyrazolyl, 1-isopropyl-3-ethyl-4-pyrazolyl, 1,3-diisopropyl-4-pyrazolyl, 1,3-dimethyl-5-pyrazolyl, 1-methyl-4-imidazolyl;
4)

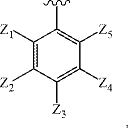

wherein Z₁, Z₂, Z₃, Z₄, Z₅ each are independently selected from the following groups, and Z₁, Z₂, Z₃, Z₄, Z₅ are not hydrogen at the same time:
   (1) H, F, Cl, Br, I, nitro, cyano,
   (2) C₁-C₆ alkyl, C₁-C₆ alkoxy, C₁-C₆ fluorine-containing alkyl, C₁-C₆ fluorine-containing alkoxy, C₂-C₆ alkenyl, C₃-C₆ cycloalkyl,
   (3) piperazinyl, N-methylpiperazinyl, N-ethylpiperazinyl, N-n-propylpiperazinyl, N-isopropylpiperazinyl, 4-(piperidyl-4-)piperazinyl, 4-(N-methylpiperidyl-4-)piperazinyl, 4-(N-ethylpiperidyl-4-)piperazinyl, 4-(N-n-propylpiperidyl-4-)piperazinyl, 4-(N-isopropylpiperidyl-4-)piperazinyl, 4-(azetidin-3-yl)piperazinyl, 4-(N-methyl-azetidin-3-yl)piperazinyl, 4-(N-ethyl-azetidin-3-yl)piperazinyl, 4-(N-n-propyl-azetidin-3-yl)piperazinyl, 4-(N-isopropyl-azetidin-3-yl)piperazinyl,
   (4) morpholinyl, 2,6-dimethylmorpholinyl, thiomorpholinyl,
   (5) 3-(N,N-dimethylamino)tetrahydropyrrolyl, 3-(N,N-diethylamino)tetrahydropyrrolyl,
   (6) 4-N,N-dimethylaminopiperidyl, 4-N,N-diethylaminopiperidyl, 4-N,N-diisopropylaminopiperidyl, 4-hydroxypiperidyl, 4-cyanopiperidyl, 4-(piperazinyl-1-)piperidyl, 4-(N-methylpiperazinyl-1-)piperidyl, 4-(N-ethylpiperazinyl-1-)piperidyl, 4-(N-isopropylpiperazinyl-1-)piperidyl, 4-(tetrahydropyrrolyl-1-)piperidyl,
   (7) piperidin-4-yl, N-methyl-4-piperidyl, N-ethyl-4-piperidyl, N-isopropyl-4-piperidyl,
   (8) aminoformamido, methylaminoformamido, ethylaminoformamido, propylaminoformamido, isopropylaminoformamido, 2-(N,N-dimethylamino)acetamido, cyclopropylsulfamido, 2-hydroxyethylamino, 2-N,N-dimethylaminoethylamino, 3-N,N-dimethylaminopropylamino, 3-N,N-diethylaminopropylamino, 3-N,N-diisopropylaminopropylamino, N-methyl-N-(2-N,N-dimethylamino)ethylamino,
   (9) 4-methyl-1-piperazinylmethylene, 4-ethyl-1-piperazinylmethylene, 4-propyl-1-piperazinylmethylene, 4-isopropyl-1-piperazinylmethylene, benzyl, cyclopropylmethylene, cyclobutylmethylene, cyclopentylmethylene, cyclohexylmethylene,
   (10) 4-(N-methyl-1-piperazinyl)piperidyl-1-formyl, 4-(N-ethyl-1-piperazinyl)piperidyl-1-formyl, N-methylpiperazinyl-1-formyl, N-ethylpiperazinyl-1-formyl, N-isopropylpiperazinyl-1-formyl,
   (11) cyclopropylaminosulfonyl, cyclobutylaminosulfonyl, cyclopentylaminosulfonyl, cyclohexylaminosulfonyl, N-methylpiperazinyl-1-sulfonyl, N-ethylpiperazinyl-1-sulfonyl, N-isopropylpiperazinyl-1-sulfonyl,
   (12) 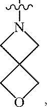 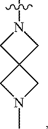 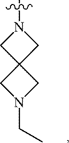 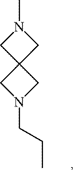 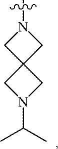 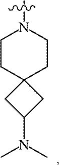 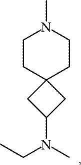 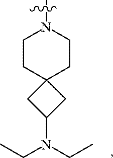 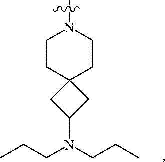 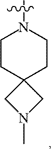 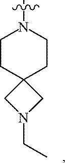 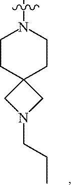 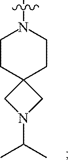
5)

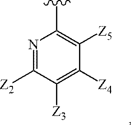

wherein Z₂, Z₃, Z₄, Z₅ each are independently selected from H, C₁-C₆ alkyl, C₁-C₆ alkoxy, piperazinyl, N-methylpiperazinyl, N-ethylpiperazinyl, N-n-propylpiperazinyl, or N-isopropylpiperazinyl, and Z₂, Z₃, Z₄, Z₅ are not hydrogen at the same time;
6)

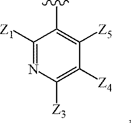

wherein Z₁, Z₃, Z₄, Z₅ each are independently selected from H, C₁-C₆ alkyl, C₁-C₆ alkoxy, N-(N-methyl-3-tetrahydropyrrolyl)amino, N-methyl-N-(N-methyl-3-tetrahydropyrrolyl)amino, N-methylpiperazinyl, N-ethylpiperazinyl, N-n-propylpiperazinyl, N-isopropylpiperazinyl, 4-(N-methylpiperazinyl-1-)piperidyl, 4-(N-ethylpiperazinyl-1-)piperidyl, or 4-(N-isopropylpiperazinyl-1-)piperidyl, and Z₁, Z₃, Z₄, Z₅ are not hydrogen at the same time;
7)

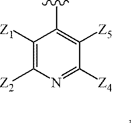

wherein Z₁, Z₂, Z₄, Z₅ each are independently selected from H, or C₁-C₆ alkyl, and Z₁, Z₂, Z₄, Z₅ may be hydrogen at the same time;
8)

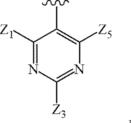

wherein Z₁, Z₃, Z₅ each are independently selected from H, C₁-C₆ alkyl, C₁-C₆ alkoxy, N-methylpiperazinyl, N-ethylpiperazinyl, N-n-propylpiperazinyl, or N-isopropylpiperazinyl, and Z₁, Z₃, Z₅ are not hydrogen at the same time;
9)

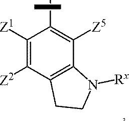

wherein Z₁, Z₂, Z₅ each are independently selected from H, C₁-C₆ alkyl, or C₁-C₆ alkoxy, and Z₁, Z₂, Z₅ may be hydrogen at the same time, R^{x} is selected from aminoformyl, methylaminoformyl, N,N-dimethylaminoformyl, ethylaminoformyl, aminoacetyl, methylaminoacetyl, 2-(N,N-dimethylamino)acetyl, or ethylaminoacetyl;
10)

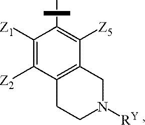

wherein Z₁, Z₂, Z₅ each are independently selected from H, F, Cl, Br, I, C₁-C₆ alkyl, C₁-C₆ alkoxy, C₁-C₆ fluorine-containing alkyl, or C₁-C₆ fluorine-containing alkoxy, and Z₁, Z₂, Z₅ may be hydrogen at the same time, R^{y} is C₁-C₆ alkyl;
11)

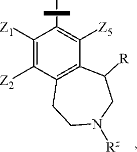

wherein Z₁, Z₂, Z₅ each are independently selected from H, or C₁-C₆ alkyl, and Z₁, Z₂, Z₅ may be hydrogen at the same time, R^{z}, R each are independently selected from C₁-C₆ alkyl.

In some embodiments, R¹ is selected from:
1) cyclopropyl, 2-N,N-dimethylaminoethyl, 2-hydroxyethyl, N-methyl-4-piperidyl, 4-aminocyclohexyl, 1-methyl-3-pyrazolyl, 1-ethyl-3-pyrazolyl, 1-isopropyl-3-pyrazolyl, 1,5-dimethyl-3-pyrazolyl, 1-methyl-4-pyrazolyl, 1-ethyl-4-pyrazolyl, 1-isopropyl-4-pyrazolyl, 1-ethyl-3-methyl-4-pyrazolyl, 1,3-dimethyl-5-pyrazolyl, 1-methyl-4-imidazolyl;
2)

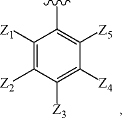

wherein Z₁, Z₂, Z₃, Z₄, Z₅ each are independently selected from the following groups, and Z₁, Z₂, Z₃, Z₄, Z₅ are not hydrogen at the same time:
   H, F, 2-hydroxyethylamino, 2-N,N-dimethylaminoethylamino, N-methyl-N-(2-N,N-dimethylamino)ethylamino, methyl, ethyl, isopropyl, cyclopropyl, methoxy, ethoxy, isopropoxy, trifluoromethyl, trifluoromethoxy, vinyl, piperazin-1-yl, N-methylpiperazinyl, 4-(N-methylpiperidyl-4-)piperazinyl, 4-(azetidin-3-yl)piperazinyl, morpholinyl, 3-(N,N-dimethylamino)tetrahydropyrrolyl, 4-N,N-dimethylaminopiperidyl, 4-hydroxypiperidyl, 4-cyanopiperidyl, 4-(N-methylpiperazinyl-1-)piperidyl, 4-(piperazinyl-1-)piperidyl, 4-(tetrahydropyrrolyl-1-)piperidyl, N-methyl-4-piperidyl, 2-(N,N-dimethylamino)acetamido, cyclopropylsulfamido, N-methyl-N-(2-N,N-dimethylamino)ethylamino, 4-methyl-1-piperazinylmethylene, benzyl, cyclopropylmethylene, 4-(N-methyl-1-piperazinyl)piperidyl-1-formyl, N-methylpiperazinyl-1-formyl, cyclopropylaminosulfonyl, N-methylpiperazinyl-1-sulfonyl, 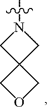 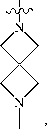 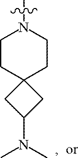 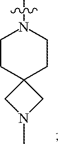
3)

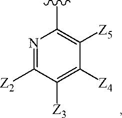

wherein Z₂, Z₃, Z₄, Z₅ each are independently selected from H, methoxy, or N-methylpiperazinyl, and Z₂, Z₃, Z₄, Z₅ are not hydrogen at the same time;
4)

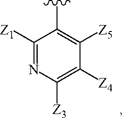

wherein Z₁, Z₃, Z₄, Z₅ each are independently selected from H, methyl, ethyl, methoxy, N-(N-methyl-3-tetrahydropyrrolyl)amino, N-methyl-N-(N-methyl-3-tetrahydropyrrolyl)amino, N-methylpiperazinyl, or 4-(N-methylpiperazinyl-1-)piperidyl, and Z₁, Z₃, Z₄, Z₅ are not hydrogen at the same time;
5)

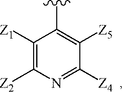

wherein Z₁, Z₂, Z₄, Z₅ are H;
6)

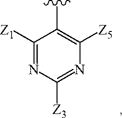

wherein Z₁, Z₃, Z₅ each are independently selected from H, methoxy, or N-methylpiperazinyl, and Z₁, Z₃, Z₅ are not hydrogen at the same time;
7)

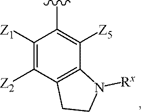

wherein Z₁, Z₂, Z₅ each are independently selected from H, or methoxy, R^{x} is 2-(N,N-dimethylamino)acetyl;
8)

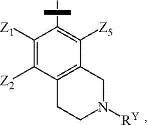

wherein Z₁, Z₂, Z₅ each are independently selected from H, F, Cl, methyl, methoxy, trifluoromethyl, or trifluoromethoxy, R^{y} is methyl;
9)

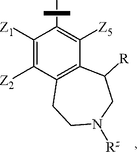

wherein Z₁, Z₂, Z₅ are H, R^{z}, R are methyl.

In some embodiments, R², R³ each are independently selected from H, methyl, trifluoromethyl, ethyl, isopropyl, or cyclopropyl.

In some embodiments, R⁴ is H, R⁵ is selected from H, F, Cl, Br, trifluoromethyl, cyano, nitro, methyl, ethyl, isopropyl, methoxy, ethoxy or cyclopropyl, or, R⁴, R⁵ and carbon atoms linked thereto together form

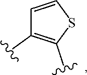

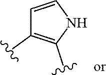

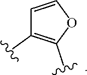

In the second aspect of the present invention, the present invention provides a compound having the general formula (I):

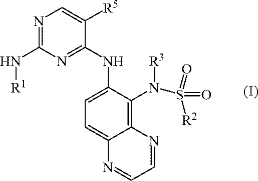

or a stereoisomer, a prodrug, a pharmaceutically acceptable salt or a pharmaceutically acceptable solvate thereof, wherein:
R¹ is selected from:
   1) H, C₁-C₆ alkyl, C₁-C₆ fluorine-containing alkyl, C₃-C₈ cycloalkyl, C₁-C₆ heteroatom-containing alkyl, C₃-C₈ heteroatom-containing cycloalkyl;
   2) 2-N,N-dimethylaminoethyl, 3-N,N-dimethylaminopropyl, 2-N,N-diethylaminoethyl, 3-N,N-diethylaminopropyl, 2-(N-methylpiperazinyl)ethyl, 2-(N-acetylpiperazinyl)ethyl, 2-morpholinylethyl, 2-thiomorpholinylethyl, 2-piperidylethyl, 2-hydroxyethyl, 3-hydroxypropyl, 2-methoxyethyl, N-methyl-4-piperidyl, N-ethyl-4-piperidyl, N-propyl-4-piperidyl, N-isopropyl-4-piperidyl, N-hydroxyethyl-4-piperidyl, N-cyanomethyl-4-piperidyl, N-(2-cyanoethyl)-4-piperidyl, N-acetyl-4-piperidyl, N-propionyl-4-piperidyl, N-isopropionyl-4-piperidyl, N-cyclopropylformyl-4-piperidyl, N-methylsulfonyl-4-piperidyl, N-ethylsulfonyl-4-piperidyl, N-propylsulfonyl-4-piperidyl, N-isopropylsulfonyl-4-piperidyl, N-cyclopropylsulfonyl-4-piperidyl, 3-N,N-dimethylam inocyclopentyl, 3-N,N-diethylam inocyclopentyl, 3-N,N-dipropylam inocyclopentyl, 3-N,N-isopropylam inocyclopentyl, 4-aminocyclohexyl, 4-N,N-dimethylam inocyclohexyl, 4-N,N-diethylam inocyclohexyl, 4-N,N-dipropylaminocyclohexyl, 4-N,N-diisopropylaminocyclohexyl, 4-acetylaminocyclohexyl, 4-propionylam inocyclohexyl, 4-isopropylformylam inocyclohexyl, 4-methylsulfonylam inocyclohexyl, 4-ethylsulfonylam inocyclohexyl, 4-propylsulfonylam inocyclohexyl, 4-isopropylsulfonylam inocyclohexyl, 4-cyclopropylsulfonylam inocyclohexyl, 4-hydroxycyclohexyl, 4-methoxycyclohexyl, 4-ethoxycyclohexyl, 4-isopropoxycyclohexyl;
   3) 1-methyl-3-pyrrolyl, 1-ethyl-3-pyrrolyl, 1-isopropyl-3-pyrrolyl, 1-cyclopropyl-3-pyrrolyl, 1-methyl-3-pyrazolyl, 1-ethyl-3-pyrazolyl, 1-isopropyl-3-pyrazolyl, 1-cyclopropyl-3-pyrazolyl, 1 -hydroxyethyl-3-pyrazolyl, 1,5-dimethyl-3-pyrazolyl, 1,5-diethyl-3-pyrazolyl, 1,5-diisopropyl-3-pyrazolyl, 1-methyl-4-pyrazolyl, 1-ethyl-4-pyrazolyl, 1 -isopropyl-4-pyrazolyl, 1,3-dimethyl-4-pyrazolyl, 1-methyl-3-ethyl-4-pyrazolyl, 1-methyl-3-isopropyl-4-pyrazolyl, 1-ethyl-3-methyl-4-pyrazolyl, 1,3-diethyl-4-pyrazolyl, 1-ethyl-3-isopropyl-4-pyrazolyl, 1-isopropyl-3-methyl-4-pyrazolyl, 1-isopropyl-3-ethyl-4-pyrazolyl, 1,3-diisopropyl-4-pyrazolyl, 1 -cyclopropyl-4-pyrazolyl, 1-hydroxyethyl-4-pyrazolyl, 1-(N-methyl-4-piperidyl)-4-pyrazolyl, 1-(N-t-butoxycarbonyl-4-piperidyl)-4-pyrazolyl, 1,3-dimethyl-5-pyrazolyl, 1-methyl-4-imidazolyl, 3-methyl-5-isoxazolyl;
   4) 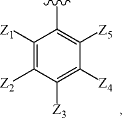
   wherein Z₁, Z₂, Z₃, Z₄, Z₅ each are independently selected from the following groups, and Z₁, Z₂, Z₃, Z₄, Z₅ are not hydrogen at the same time:
      (1) H, F, Cl, Br, l, nitro, cyano,
      (2) C₁-C₆ alkyl, C₁-C₆ alkoxy, C₁-C₆ oxygen-containing alkyl, C₁-C₆ fluorine-containing alkyl, C₁-C₆ fluorine-containing alkoxy, C₃-C₆ cycloalkyl, C₂-C₆ alkenyl,
      (3) 2-N,N-dimethylaminoethoxy, 3-N,N-dimethylaminopropoxy, 2-(N-methylpiperazinyl)ethoxy, 2-(N-acetylpiperazinyl)ethoxy, 2-morpholinylethoxy, 2-thiomorpholinylethoxy, 2-piperidylethoxy, 3-N,N-dimethylaminopropoxy, 3-N,N-diethylaminopropoxy, 3-N,N-diisopropylaminopropoxy, 3-(N-methylpiperazinyl)propoxy, 3-(N-acetylpiperazinyl)propoxy, 3-morpholinylpropoxy, 3-thiomorpholinylpropoxy, 3-piperidylpropoxy, 2-pyridylmethoxy, 3-pyridylmethoxy, 4-pyridylmethoxy, phenylmethoxy, monohalo-substituted phenylmethoxy, gem-dihalo-substituted phenylmethoxy, hetero-dihalo-substituted phenylmethoxy,
      (4) piperazinyl, N-methylpiperazinyl, N-ethylpiperazinyl, N-n-propylpiperazinyl, N-isopropylpiperazinyl, 4-(piperidyl-4-)piperazinyl, 4-(N-methylpiperidyl-4-)piperazinyl, 4-(N-ethylpiperidyl-4-)piperazinyl, 4-(N-n-propylpiperidyl-4-)piperazinyl, 4-(N-isopropylpiperidyl-4-)piperazinyl, 4-(azetidin-3-yl)piperazinyl, 4-(N-methyl-azetidin-3-yl)piperazinyl, 4-(N-ethyl-azetidin-3-yl)piperazinyl, 4-(N-n-propyl-azetidin-3-yl)piperazinyl, 4-(N-isopropyl-azetidin-3-yl)piperazinyl, N-cyclopropylpiperazinyl, N-(2-hydroxyethyl)piperazinyl, N-(2-N,N-dimethylaminoethyl)piperazinyl, N-(2-N,N-diethylaminoethyl)piperazinyl, N-(2-cyanoethyl)piperazinyl, N-cyanomethylpiperazinyl, N-(3-hydroxypropyl)piperazinyl, N-(3-N,N-dimethylaminopropyl)piperazinyl, N-(3-N,N-diethylaminopropyl)piperazinyl, N-(N-methyl-4-piperidyl)piperazinyl, N-(N-ethyl-4-piperidyl)piperazinyl, N-t-butoxycarbonylpiperazinyl, N-acetylpiperazinyl, N-propionylpiperazinyl, N-isobutyrylpiperazinyl, N-cyclopropylformylpiperazinyl, N-methylsulfonylpiperazinyl, N-ethylsulfonylpiperazinyl, N-n-propylsulfonylpiperazinyl, N-isopropylsulfonylpiperazinyl, N-cyclopropylsulfonylpiperazinyl, 2-oxo-piperazin-4-yl,
      (5) morpholinyl, 2,6-dimethylmorpholinyl, thiomorpholinyl,
      (6) tetrahydropyrrolyl, 3-(N,N-dimethylamino)tetrahydropyrrolyl, 3-(N, N-diethylam ino)tetrahydropyrrolyl,
      (7) imidazolyl, 4-methyl-1-imidazolyl,
      (8) piperidyl, 4-N,N-dimethylaminopiperidyl, 4-N,N-diethylaminopiperidyl, 4-N,N-diisopropylaminopiperidyl, 4-hydroxypiperidyl, 4-cyanopiperidyl, 4-methoxypiperidyl, 4-ethoxypiperidyl, 4-(piperazinyl-1 -)piperidyl, 4-(N-methylpiperazinyl-1 -)piperidyl, 4-(N-ethylpiperazinyl-1 -)piperidyl, 4-(N-isopropylpiperazinyl-1-)piperidyl, 4-(N-acetylpiperazinyl-1-)piperidyl, 4-(N-t-butoxycarbonylpiperazinyl-1-)piperidyl, 4-(N-methylsulfonylpiperazinyl-1-)piperidyl, 4-(N-(2-hydroxyethyl)piperazinyl-1-)piperidyl, 4-(N-(2-cyanoethyl)piperazinyl-1-)piperidyl, 4-(N-(3-hydroxypropyl)piperazinyl-1-)piperidyl, 4-(N-(2-N, N-dimethylaminoethyl)piperazinyl-1 -)piperidyl, 4-(N-(2-N,N-diethylethyl)piperazinyl-1 -)piperidyl, 4-(N-(3-N,N-dimethylpropyl)piperazinyl-1 -)piperidyl, 4-(N-(3-N,N-diethylpropyl)piperazinyl-1-)piperidyl, 4-(tetrahydropyrrolyl-1 -)piperidyl, 4-(3-N, N-dimethylaminotetrahydropyrrolyl-1 -)piperidyl, 4-(3-N, N-diethylaminotetrahydropyrrolyl-1 -)piperidyl,
      (9) piperidin-4-yl, N-methyl-4-piperidyl, N-ethyl-4-piperidyl, N-isopropyl-4-piperidyl, N-acetyl-4-piperidyl, N-t-butoxycarbonyl-4-piperidyl, N-methylsulfonyl-4-piperidyl, N-(2-hydroxyethyl)-4-piperidyl, N-(2-N, N-dimethylaminoethyl)-4-piperidyl, N-(2-N, N-diethylaminoethyl)-4-piperidyl, N-(2-cyanoethyl)-4-piperidyl, N-(3-hydroxypropyl)-4-piperidyl, N-(3-N, N-dimethylaminopropyl)-4-piperidyl, N-(3-N, N-diethylaminopropyl)-4-piperidyl, N-(3-cyanopropyl)-4-piperidyl, N-cyanomethylene-4-piperidyl,
      (10) amino, N,N-dimethylamino, N,N-diethylamino, N,N-di-n-propylamino, N,N-diisopropylamino, 2-hydroxyethylamino, 2-N,N-dimethylaminoethylamino, 2-morpholinylethylamino, 2-(N-methylpiperazinyl)ethylamino, 3-N, N-dimethylaminopropylamino, 3-N, N-diethylaminopropylamino, 3-N, N-diisopropylam inopropylam ino, 3-morpholinylpropylamino, 3- (N-methylpiperazinyl)propylamino, N-methylpiperidyl-4-amino, N-ethylpiperidyl-4-amino, N-n-propylpiperidyl-4-amino, N-isopropylpiperidyl-4-amino, acetamido, propionamido, 2-(N,N-dimethylamino)acetamido, 2-hydroxyacetamido, 2-methoxyacetamido, methylsulfamido, ethylsulfamido, n-propylsulfamido, isopropylsulfamido, cyclopropylsulfamido, N-methyl-N-(N-methyl-3-tetrahydropyrrolyl)amino, N-(N-methyl-3-tetrahydropyrrolyl)amino, N-methyl-N-(2-N,N-dimethylamino)ethylamino,
      (11) aminomethylene, N,N-dimethylaminomethylene, N,N-diethylaminomethylene, 4-methyl-1-piperazinylmethylene, 4-ethyl-1-piperazinylmethylene, 4-propyl-1-piperazinylmethylene, 4-isopropyl-1-piperazinylmethylene, 4-(2-hydroxyethyl)-1-piperazinylmethylene, 4-(2-N,N-dimethylaminoethyl)-1-piperazinylmethylene, piperidyl-1-methylene, 4-(N,N-dimethylamino)-1-piperidylmethylene, 4-hydroxypiperidyl-1-methylene, tetrahydropyrrolyl-1-methylene, 3-(N,N-dimethylamino)-1-tetrahydropyrrolylmethylene, benzyl, cyclopropylmethylene, cyclobutylmethylene, cyclopentylmethylene, cyclohexylmethylene,
      (12) aminoformyl, methylaminoformyl, N,N-dimethylaminoformyl, ethylaminoformyl, cyclopropylaminoformyl, cyclobutylaminoformyl, cyclopentylaminoformyl, cyclohexylaminoformyl, aminoacetyl, methylaminoacetyl, 2-(N,N-dimethylamino)acetyl, ethylaminoacetyl, cyclopropylam inoacetyl, cyclobutylaminoacetyl, cyclopentylaminoacetyl, cyclohexylaminoacetyl, tetrahydropyrrolyl-1-formyl, 3-(N, N-dimethylamino)tetrahydropyrrolyl-1 -formyl, 3- (N,N-diethylamino)tetrahydropyrrolyl-1-formyl, morpholinyl-4-formyl, thiomorpholinyl-4-formyl, 2,6-dimethylmorpholinyl-4-formyl, piperidyl-1-formyl, 4-hydroxypiperidyl-1-formyl, 4-(N,N-dimethylamino)piperidyl-1-formyl, 4-(N,N-diethylamino)piperidyl-1-formyl, 4-(N-methyl-1-piperazinyl)piperidyl-1-formyl, 4-(N-ethyl-1-piperazinyl)piperidyl-1-formyl, N-methylpiperazinyl-1-formyl, N-ethylpiperazinyl-1-formyl, N-isopropylpiperazinyl-1-formyl, N-(2-hydroxyethyl)piperazinyl-1-formyl, N-(2-N,N-dimethylam inoethyl)piperazinyl-1-formyl, N-(2-N,N-diethylam inoethyl)piperazinyl-1-formyl, 4-(N-methyl-4-piperidyl)piperazinyl-1-formyl, 4-(N-ethyl-4-piperidyl)piperazinyl-1-formyl,
      (13) hydroxyformyl, methoxyformyl, ethoxyformyl, propoxyformyl, isopropoxyformyl, n-butoxyformyl, iso-butoxyformyl, t-butoxyformyl,
      (14) hydroxysulfonyl, aminosulfonyl, methylaminosulfonyl, ethylaminosulfonyl, propylaminosulfonyl, isopropylaminosulfonyl, cyclopropylaminosulfonyl, cyclobutylaminosulfonyl, cyclopentylaminosulfonyl, cyclohexylaminosulfonyl, tetrahydropyrrolyl-1-sulfonyl, 3-(N,N-dimethylamino)tetrahydropyrrolyl-1-sulfonyl, 3-(N, N-diethylam ino)tetrahydropyrrolyl-1-sulfonyl, morpholinyl-4-sulfonyl, thiomorpholinyl-4-sulfonyl, 2,6-dimethylmorpholinyl-4-sulfonyl, piperidyl-1-sulfonyl, 4-hydroxypiperidyl-1-sulfonyl, 4-(N,N-dimethylamino)piperidyl-1-sulfonyl, 4-(N,N-diethylamino)piperidyl-1-sulfonyl, 4-(N-methyl-1-piperazinyl)piperidyl-1-sulfonyl, 4-(N-ethyl-1-piperazinyl)piperidyl-1-sulfonyl, N-methylpiperazinyl-1-sulfonyl, N-ethylpiperazinyl-1-sulfonyl, N-isopropylpiperazinyl-1-sulfonyl, N-(2-hydroxyethyl)piperazinyl-1-sulfonyl, N-(2-N,N-dimethylam inoethyl)piperazinyl-1-sulfonyl, N-(2-N,N-diethylam inoethyl)piperazinyl-1-sulfonyl, 4-(N-methyl-4-piperidyl)piperazinyl-1-sulfonyl, 4-(N-ethyl-4-piperidyl)piperazinyl-1-sulfonyl,
      (15) aminoformamido, methylaminoformamido, ethylaminoformamido, propylam inoformam ido, isopropylam inoformam ido, cyclopropylam inoformam ido, cyclobutylaminoformamido, cyclopentylaminoformamido, piperidyl-1-formamido, 4-hydroxypiperidyl-1-formamido, 4-N,N-dimethylpiperidyl-1-formamido, 4-N, N-diethylpiperidyl-1 -formamido, tetrahydropyrrolyl-1 -formamido, 3-N, N-dimethyltetrahydropyrrolyl-1-formam ido, 3-N,N-diethyltetrahydropyrrolyl-1-formamido, N-methylpiperazinyl-1-formam ido, N-ethylpiperazinyl-1 -formamido, N-acetylpiperazinyl-1 -formam ido, N-t-butoxycarbonylpiperazinyl-1-formamido, N-(2-hydroxyethyl)piperazinyl-1-formamido, N-(2-cyanoethyl)piperazinyl-1-formam ido, N-(2-N, N-dimethylam inoethyl)piperazinyl-1-formam ido, N-(2-N, N-diethylam inoethyl)piperazinyl-1-formam ido, N-(3-hydroxypropyl)piperazinyl-1-formam ido, N-(3-N,N-dimethylpropyl)piperazinyl-1 -formamido, N-(3-N,N-diethylpropyl)piperazinyl-1-formamido, morpholinyl-1-formamido, 3,5-dimethylmorpholinyl-1-formamido, 4-(tetrahydropyrrolyl)piperidyl-1-formamido, 4-(N-methyl-1-piperazinyl)piperidyl-1-formam ido, 4-(N-ethyl-1-piperazinyl)piperidyl-1-formamido, 4-(N-acetyl-1-piperazinyl)piperidyl-1-formamido, N-(N-methyl-4-piperidyl)piperazinyl-1-formamido,
      (16) 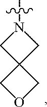 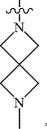 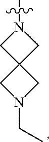 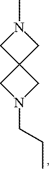 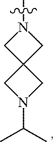 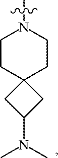 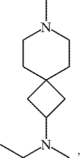 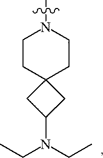 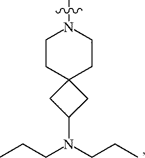 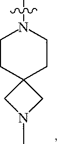 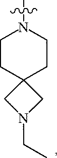 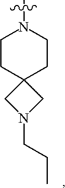 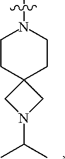
      (17) Z₃ and Z₄ may form an oxygen-containing substituted or unsubstituted 5- to 7-membered ring; the substituent may be selected from the same substituents as described above for Z₁,
      (18) Z₃ and Z₄ may form a nitrogen-containing substituted or unsubstituted 5- to 7-membered ring; the substituent may be selected from the same substituents as described above for Z₁;
   5) 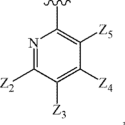
   wherein Z₂, Z₃, Z₄, Z₅ are the same as defined in 4), and Z₂, Z₃, Z₄, Z₅ are not hydrogen at the same time;
   6) 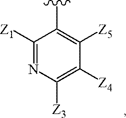
   wherein Z₁, Z₃, Z₄, Z₅ are the same as defined in 4), and Z₁, Z₃, Z₄, Z₅ are not hydrogen at the same time;
   7) 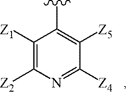
   wherein Z₁, Z₂, Z₄, Z₅ are the same as defined in 4), and Z₁, Z₂, Z₄, Z₅ may be hydrogen at the same time;
   8) 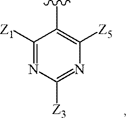
   wherein Z₁, Z₃, Z₅ are the same as defined in 4), and Z₁, Z₃, Z₅ are not hydrogen at the same time;
R², R³ each are independently selected from H, C₁-C₆ alkyl, C₁-C₆ fluorine-containing alkyl, C₃-C₆ cycloalkyl, or C₃-C₆ fluorine-containing cycloalkyl;
R⁵ is selected from H, F, Cl, Br, C₁-C₆ fluorine-containing alkyl, cyano, nitro, C₁-C₆ alkyl, C₁-C₆ alkoxy, or C₃-C₆ cycloalkyl.

In some embodiments, R¹ is selected from:
1) H, C₁-C₆ alkyl, C₁-C₆ fluorine-containing alkyl, C₃-C₈ cycloalkyl, C₁-C₆ heteroatom-containing alkyl, C₃-C₈ heteroatom-containing cycloalkyl;
2) 2-N,N-dimethylaminoethyl, 3-N,N-dimethylaminopropyl, 2-N,N-diethylaminoethyl, 3-N,N-diethylaminopropyl, 2-(N-methylpiperazinyl)ethyl, 2-(N-acetylpiperazinyl)ethyl, 2-morpholinylethyl, 2-thiomorpholinylethyl, 2-piperidylethyl, 2-hydroxyethyl, 3-hydroxypropyl, 2-methoxyethyl, N-methyl-4-piperidyl, N-ethyl-4-piperidyl, N-propyl-4-piperidyl, N-isopropyl-4-piperidyl, N-hydroxyethyl-4-piperidyl, N-cyanomethyl-4-piperidyl, N-(2-cyanoethyl)-4-piperidyl, 3-N,N-dimethylaminocyclopentyl, 3-N,N-diethylaminocyclopentyl, 3-N,N-dipropylaminocyclopentyl, 3-N,N-isopropylaminocyclopentyl, 4-aminocyclohexyl, 4-N, N-dimethylam inocyclohexyl, 4-N, N-diethylam inocyclohexyl, 4-N,N-dipropylaminocyclohexyl, 4-N,N-diisopropylaminocyclohexyl, 4-hydroxycyclohexyl, 4-methoxycyclohexyl, 4-ethoxycyclohexyl, 4-isopropoxycyclohexyl;
3) 1-methyl-3-pyrrolyl, 1-ethyl-3-pyrrolyl, 1-isopropyl-3-pyrrolyl, 1-cyclopropyl-3-pyrrolyl, 1 -methyl-3-pyrazolyl, 1-ethyl-3-pyrazolyl, 1-isopropyl-3-pyrazolyl, 1-cyclopropyl-3-pyrazolyl, 1-hydroxyethyl-3-pyrazolyl, 1,5-dimethyl-3-pyrazolyl, 1,5-diethyl-3-pyrazolyl, 1,5-diisopropyl-3-pyrazolyl, 1-methyl-4-pyrazolyl, 1-ethyl-4-pyrazolyl, 1-isopropyl-4-pyrazolyl, 1-cyclopropyl-4-pyrazolyl, 1-hydroxyethyl-4-pyrazolyl, 1,3-dimethyl-4-pyrazolyl, 1-methyl-3-ethyl-4-pyrazolyl, 1-methyl-3-isopropyl-4-pyrazolyl, 1-ethyl-3-methyl-4-pyrazolyl, 1,3-diethyl-4-pyrazolyl, 1-ethyl-3-isopropyl-4-pyrazolyl, 1-isopropyl-3-methyl-4-pyrazolyl, 1-isopropyl-3-ethyl-4-pyrazolyl, 1,3-diisopropyl-4-pyrazolyl, 1,3-dimethyl-5-pyrazolyl, 1-methyl-4-imidazolyl, 3-methyl-5-isoxazolyl;
4)

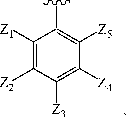

wherein Z₁, Z₂, Z₃, Z₄, Z₅ each are independently selected from the following groups, and Z₁, Z₂, Z₃, Z₄, Z₅ are not hydrogen at the same time:
   (1) H, F, Cl, Br, l, nitro, cyano,
   (2) C₁-C₆ alkyl, C₁-C₆ alkoxy, C₁-C₆ oxygen-containing alkyl, C₁-C₆ fluorine-containing alkyl, C₁-C₆ fluorine-containing alkoxy, C₃-C₆ cycloalkyl, C₂-C₆ alkenyl,
   (3) piperazinyl, N-methylpiperazinyl, N-ethylpiperazinyl, N-n-propylpiperazinyl, N-isopropylpiperazinyl, 4-(piperidyl-4-)piperazinyl, 4-(N-methylpiperidyl-4-)piperazinyl, 4-(N-ethylpiperidyl-4-)piperazinyl, 4-(N-n-propylpiperidyl-4-)piperazinyl, 4-(N-isopropylpiperidyl-4-)piperazinyl, 4-(azetidin-3-yl)piperazinyl, 4-(N-methyl-azetidin-3-yl)piperazinyl, 4-(N-ethyl-azetidin-3-yl)piperazinyl, 4-(N-n-propyl-azetidin-3-yl)piperazinyl, 4-(N-isopropyl-azetidin-3-yl)piperazinyl, N-cyclopropylpiperazinyl, N-(2-hydroxyethyl)piperazinyl, N-(2-N,N-dimethylaminoethyl)piperazinyl, N-(2-N,N-diethylaminoethyl)piperazinyl, N-(2-cyanoethyl)piperazinyl, N-cyanomethylpiperazinyl, N-(3-hydroxypropyl)piperazinyl, N-(3-N,N-dimethylaminopropyl)piperazinyl, N-(3-N,N-diethylaminopropyl)piperazinyl,
   (4) morpholinyl, 2,6-dimethylmorpholinyl, thiomorpholinyl,
   (5) tetrahydropyrrolyl, 3-(N,N-dimethylamino)tetrahydropyrrolyl, 3-(N, N-diethylam ino)tetrahydropyrrolyl,
   (6) imidazolyl, 4-methyl-1-imidazolyl,
   (7) piperidyl, 4-N,N-dimethylaminopiperidyl, 4-N,N-diethylaminopiperidyl, 4-N,N-diisopropylaminopiperidyl, 4-hydroxypiperidyl, 4-cyanopiperidyl, 4-methoxypiperidyl, 4-ethoxypiperidyl, 4-(piperazinyl-1 -)piperidyl, 4-(N-methylpiperazinyl-1 -)piperidyl, 4-(N-ethylpiperazinyl-1 -)piperidyl, 4-(N-isopropylpiperazinyl-1 -)piperidyl, 4-(N-(2-hydroxyethyl)piperazinyl-1 -)piperidyl, 4-(N-(2-cyanoethyl)piperazinyl-1 -)piperidyl, 4-(N-(3-hydroxypropyl)piperazinyl-1 -)piperidyl, 4-(N-(2-N, N-dimethylam inoethyl)piperazinyl-1 -)piperidyl, 4-(N-(2-N,N-diethylethyl)piperazinyl-1 -)piperidyl, 4-(N-(3-N,N-dimethylpropyl)piperazinyl-1 -)piperidyl, 4-(N-(3-N,N-diethylpropyl)piperazinyl-1-)piperidyl, 4-(tetrahydropyrrolyl-1-)piperidyl, 4-(3-N, N-dimethylam inotetrahydropyrrolyl-1 -)piperidyl, 4-(3-N, N-diethylam inotetrahydropyrrolyl-1 -)piperidyl,
   (8) piperidin-4-yl, N-methyl-4-piperidyl, N-ethyl-4-piperidyl, N-isopropyl-4-piperidyl, N-(2-hydroxyethyl)-4-piperidyl, N-(2-N, N-dimethylam inoethyl)-4-piperidyl, N-(2-N, N-diethylam inoethyl)-4-piperidyl, N-(2-cyanoethyl)-4-piperidyl, N-(3-hydroxypropyl)-4-piperidyl, N-(3-N, N-dimethylam inopropyl)-4-piperidyl, N-(3-N, N-diethylam inopropyl)-4-piperidyl, N-(3-cyanopropyl)-4-piperidyl, N-cyanomethylene-4-piperidyl,
   (9) amino, N,N-dimethylamino, N,N-diethylamino, N,N-di-n-propylamino, N,N-diisopropylamino, 2-hydroxyethylamino, 2-N,N-dimethylaminoethylamino, 3-N, N-dimethylam inopropylam ino, 3-N, N-diethylam inopropylam ino, 3-N, N-diisopropylam inopropylam ino, N-methylpiperidyl-4-am ino, N-ethylpiperidyl-4-amino, N-n-propylpiperidyl-4-amino, N-isopropylpiperidyl-4-amino, acetamido, propionamido, 2-(N,N-dimethylamino)acetamido, 2-hydroxyacetamido, 2-methoxyacetamido, methylsulfamido, ethylsulfamido, n-propylsulfamido, isopropylsulfamido, cyclopropylsulfamido, N-methyl-N-(N-methyl-3-tetrahydropyrrolyl)amino, N-(N-methyl-3-tetrahydropyrrolyl)amino, N-methyl-N-(2-N,N-dimethylamino)ethylamino,
   (10) 4-methyl-1-piperazinylmethylene, 4-ethyl-1-piperazinylmethylene, 4-propyl-1-piperazinylmethylene, 4-isopropyl-1-piperazinylmethylene, 4-(2-hydroxyethyl)-1-piperazinylmethylene, 4-(2-N,N-dimethylam inoethyl)-1-piperazinylmethylene, piperidyl-1-methylene, 4-(N,N-dimethylamino)-1-piperidylmethylene, 4-hydroxypiperidyl-1-methylene, tetrahydropyrrolyl-1-methylene, 3-(N,N-dimethylamino)-1-tetrahydropyrrolylmethylene, benzyl, cyclopropylmethylene, cyclobutylmethylene, cyclopentylmethylene, cyclohexylmethylene,
   (11) aminoformyl, methylaminoformyl, N,N-dimethylaminoformyl, ethylaminoformyl, aminoacetyl, methylaminoacetyl, 2-(N,N-dimethylamino)acetyl, ethylaminoacetyl, tetrahydropyrrolyl-1-formyl, 3-(N, N-dimethylam ino)tetrahydropyrrolyl-1 -formyl, 3- (N,N-diethylamino)tetrahydropyrrolyl-1-formyl, morpholinyl-4-formyl, thiomorpholinyl-4-formyl, 2,6-dimethylmorpholinyl-4-formyl, piperidyl-1-formyl, 4-hydroxypiperidyl-1-formyl, 4-(N,N-dimethylamino)piperidyl-1-formyl, 4-(N,N-diethylamino)piperidyl-1-formyl, 4-(N-methyl-1-piperazinyl)piperidyl-1-formyl, 4-(N-ethyl-1-piperazinyl)piperidyl-1-formyl, N-methylpiperazinyl-1-formyl, N-ethylpiperazinyl-1-formyl, N-isopropylpiperazinyl-1-formyl, N-(2-hydroxyethyl)piperazinyl-1-formyl, N-(2-N, N-dimethylam inoethyl)piperazinyl-1-formyl, N-(2-N, N-diethylam inoethyl)piperazinyl-1-formyl, 4-(N-methyl-4-piperidyl)piperazinyl-1-formyl, 4-(N-ethyl-4-piperidyl)piperazinyl-1-formyl,
   (12) hydroxysulfonyl, aminosulfonyl, methylaminosulfonyl, ethylaminosulfonyl, propylaminosulfonyl, isopropylaminosulfonyl, cyclopropylaminosulfonyl, cyclobutylaminosulfonyl, cyclopentylaminosulfonyl, cyclohexylaminosulfonyl, tetrahydropyrrolyl-1-sulfonyl, 3-(N,N-dimethylamino)tetrahydropyrrolyl-1-sulfonyl, 3- (N,N-diethylamino)tetrahydropyrrolyl-1-sulfonyl, morpholinyl-4-sulfonyl, thiomorpholinyl-4-sulfonyl, 2,6-dimethylmorpholinyl-4-sulfonyl, piperidyl-1-sulfonyl, 4-hydroxypiperidyl-1-sulfonyl, 4-(N,N-dimethylamino)piperidyl-1-sulfonyl, 4-(N,N-diethylamino)piperidyl-1-sulfonyl, N-methylpiperazinyl-1-sulfonyl, N-ethylpiperazinyl-1-sulfonyl, N-isopropylpiperazinyl-1-sulfonyl, N-(2-hydroxyethyl)piperazinyl-1-sulfonyl, N-(2-N,N-dimethylam inoethyl)piperazinyl-1-sulfonyl, N-(2-N,N-diethylam inoethyl)piperazinyl-1-sulfonyl,
   (13) aminoformamido, methylaminoformamido, ethylaminoformamido, propylaminoformamido, isopropylaminoformamido,
   (14) 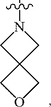 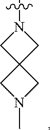 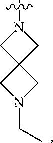 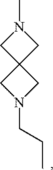 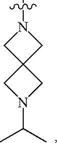 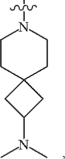 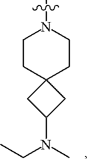 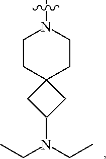 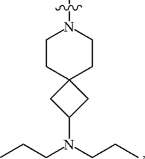 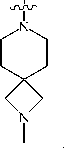 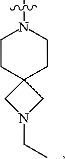 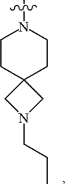 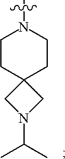
5)

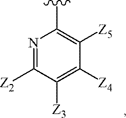

wherein Z₂, Z₃, Z₄, Z₅ are the same as defined in 4), and Z₂, Z₃, Z₄, Z₅ are not hydrogen at the same time;
6)

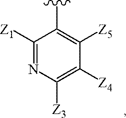

wherein Z₁, Z₃, Z₄, Z₅ are the same as defined in 4), and Z₁, Z₃, Z₄, Z₅ are not hydrogen at the same time;
7)

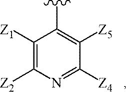

wherein Z₁, Z₂, Z₄, Z₅ are the same as defined in 4), and Z₁, Z₂, Z₄, Z₅ may be hydrogen at the same time;
8)

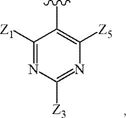

wherein Z₁, Z₃, Z₅ are the same as defined in 4), and Z₁, Z₃, Z₅ are not hydrogen at the same time;
9)

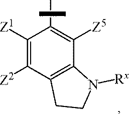

wherein Z₁, Z₂, Z₅ are the same as defined in 4), and Z₁, Z₂, Z₅ may be hydrogen at the same time, R^{X} is selected from the same substituents as described above for Z¹;
10)

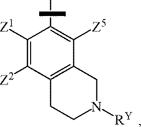

wherein Z₁, Z₂, Z₅ are the same as defined in 4), and Z₁, Z₂, Z₅ may be hydrogen at the same time, R^{y} is selected from the same substituents as described above for Z¹;
11)

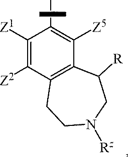

wherein Z₁, Z₂, Z₅ are the same as defined in 4), and Z₁, Z₂, Z₅ may be hydrogen at the same time, R^{z}, R each are independently selected from the same substituents as described above for Z¹.

In some embodiments, R¹ is selected from:
1) H, C₁-C₆ alkyl, C₃-C₈ cycloalkyl;
2) 2-N,N-dimethylaminoethyl, 3-N,N-dimethylaminopropyl, 2-N,N-diethylaminoethyl, 3-N,N-diethylaminopropyl, 2-hydroxyethyl, 3-hydroxypropyl, N-methyl-4-piperidyl, N-ethyl-4-piperidyl, N-propyl-4-piperidyl, N-isopropyl-4-piperidyl, 4-aminocyclohexyl, 4-N,N-dimethylaminocyclohexyl, 4-N,N-diethylaminocyclohexyl, 4-N,N-dipropylaminocyclohexyl, 4-N,N-diisopropylaminocyclohexyl;
3) 1-methyl-3-pyrazolyl, 1-ethyl-3-pyrazolyl, 1-isopropyl-3-pyrazolyl, 1-cyclopropyl-3-pyrazolyl, 1,5-dimethyl-3-pyrazolyl, 1,5-diethyl-3-pyrazolyl, 1,5-diisopropyl-3-pyrazolyl, 1 -methyl-4-pyrazolyl, 1-ethyl-4-pyrazolyl, 1-isopropyl-4-pyrazolyl, 1,3-dimethyl-4-pyrazolyl, 1-methyl-3-ethyl-4-pyrazolyl, 1-methyl-3-isopropyl-4-pyrazolyl, 1-ethyl-3-methyl-4-pyrazolyl, 1,3-diethyl-4-pyrazolyl, 1-ethyl-3-isopropyl-4-pyrazolyl, 1-isopropyl-3-methyl-4-pyrazolyl, 1-isopropyl-3-ethyl-4-pyrazolyl, 1,3-diisopropyl-4-pyrazolyl, 1-cyclopropyl-4-pyrazolyl, 1,3-dimethyl-5-pyrazolyl, 1-methyl-4-imidazolyl;
4)

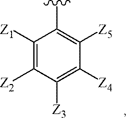

wherein Z₁, Z₂, Z₃, Z₄, Z₅ each are independently selected from the following groups, and Z₁, Z₂, Z₃, Z₄, Z₅ are not hydrogen at the same time:
   (1) H, F, Cl, Br, l, nitro, cyano,
   (2) C₁-C₆ alkyl, C₁-C₆ alkoxy, C₁-C₆ fluorine-containing alkyl, C₁-C₆ fluorine-containing alkoxy, C₂-C₆ alkenyl, C₃-C₆ cycloalkyl,
   (3) piperazinyl, N-methylpiperazinyl, N-ethylpiperazinyl, N-n-propylpiperazinyl, N-isopropylpiperazinyl, 4-(piperidyl-4-)piperazinyl, 4-(N-methylpiperidyl-4-)piperazinyl, 4-(N-ethylpiperidyl-4-)piperazinyl, 4-(N-n-propylpiperidyl-4-)piperazinyl, 4-(N-isopropylpiperidyl-4-)piperazinyl, 4-(azetidin-3-yl)piperazinyl, 4-(N-methyl-azetidin-3-yl)piperazinyl, 4-(N-ethyl-azetidin-3-yl)piperazinyl, 4-(N-n-propyl-azetidin-3-yl)piperazinyl, 4-(N-isopropyl-azetidin-3-yl)piperazinyl,
   (4) morpholinyl, 2,6-dimethylmorpholinyl, thiomorpholinyl,
   (5) 3-(N,N-dimethylamino)tetrahydropyrrolyl, 3-(N,N-diethylamino)tetrahydropyrrolyl,
   (6) 4-N,N-dimethylaminopiperidyl, 4-N,N-diethylaminopiperidyl, 4-N,N-diisopropylaminopiperidyl, 4-hydroxypiperidyl, 4-cyanopiperidyl, 4-(piperazinyl-1 -)piperidyl, 4-(N-methylpiperazinyl-1 -)piperidyl, 4-(N-ethylpiperazinyl-1 -)piperidyl, 4-(N-isopropylpiperazinyl-1 -)piperidyl, 4-(tetrahydropyrrolyl-1 -)piperidyl,
   (7) piperidin-4-yl, N-methyl-4-piperidyl, N-ethyl-4-piperidyl, N-isopropyl-4-piperidyl,
   (8) aminoformamido, methylaminoformamido, ethylaminoformamido, propylaminoformamido, isopropylaminoformamido, 2-(N,N-dimethylamino)acetamido, cyclopropylsulfamido, 2-hydroxyethylamino, 2-N,N-dimethylaminoethylamino, 3-N,N-dimethylaminopropylamino, 3-N,N-diethylaminopropylamino, 3-N,N-diisopropylaminopropylamino, N-methyl-N-(2-N,N-dimethylamino)ethylamino,
   (9) 4-methyl-1-piperazinylmethylene, 4-ethyl-1-piperazinylmethylene, 4-propyl-1-piperazinylmethylene, 4-isopropyl-1-piperazinylmethylene, benzyl, cyclopropylmethylene, cyclobutylmethylene, cyclopentylmethylene, cyclohexylmethylene,
   (10) 4-(N-methyl-1-piperazinyl)piperidyl-1-formyl, 4-(N-ethyl-1-piperazinyl)piperidyl-1-formyl, N-methylpiperazinyl-1-formyl, N-ethylpiperazinyl-1-formyl, N-isopropylpiperazinyl-1-formyl,
   (11) cyclopropylaminosulfonyl, cyclobutylaminosulfonyl, cyclopentylaminosulfonyl, cyclohexylam inosulfonyl, N-methylpiperazinyl-1-sulfonyl, N-ethylpiperazinyl-1-sulfonyl, N-isopropylpiperazinyl-1-sulfonyl,
   (12) 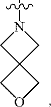 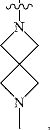 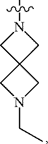 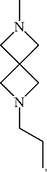 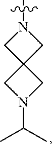 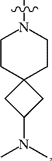 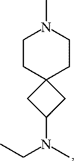 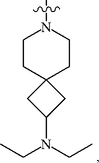 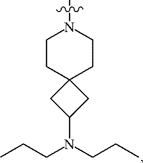 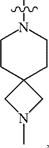 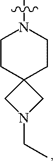 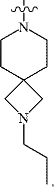 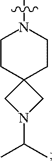
5)

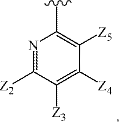

wherein Z₂, Z₃, Z₄, Z₅ each are independently selected from H, C₁-C₆ alkyl, C₁-C₆ alkoxy, piperazinyl, N-methylpiperazinyl, N-ethylpiperazinyl, N-n-propylpiperazinyl, or N-isopropylpiperazinyl, and Z₂, Z₃, Z₄, Z₅ are not hydrogen at the same time;
6)

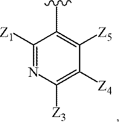

wherein Z₁, Z₃, Z₄, Z₅ each are independently selected from H, C₁-C₆ alkyl, C₁-C₆ alkoxy, N-(N-methyl-3-tetrahydropyrrolyl)amino, N-methyl-N-(N-methyl-3-tetrahydropyrrolyl)amino, N-methylpiperazinyl, N-ethylpiperazinyl, N-n-propylpiperazinyl, N-isopropylpiperazinyl, 4-(N-methylpiperazinyl-1 -)piperidyl, 4-(N-ethylpiperazinyl-1 -)piperidyl, or 4-(N-isopropylpiperazinyl-1-)piperidyl, and Z₁, Z₃, Z₄, Z₅ are not hydrogen at the same time;
7)

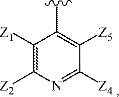

wherein Z₁, Z₂, Z₄, Z₅ each are independently selected from H, or C₁-C₆ alkyl, and Z₁, Z₂, Z₄, Z₅ may be hydrogen at the same time;
8)

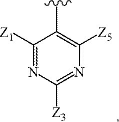

wherein Z₁, Z₃, Z₅ each are independently selected from H, C₁-C₆ alkyl, C₁-C₆ alkoxy, N-methylpiperazinyl, N-ethylpiperazinyl, N-n-propylpiperazinyl, or N-isopropylpiperazinyl, and Z₁, Z₃, Zs are not hydrogen at the same time;
9)

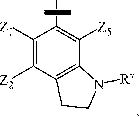

wherein Z₁, Z₂, Z₅ each are independently selected from H, C₁-C₆ alkyl, or C₁-C₆ alkoxy, and Z₁, Z₂, Z₅ may be hydrogen at the same time, R^{X} is selected from aminoformyl, methylaminoformyl, N,N-dimethylaminoformyl, ethylaminoformyl, aminoacetyl, methylaminoacetyl, 2-(N,N-dimethylamino)acetyl, or ethylaminoacetyl;
10)

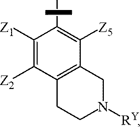

wherein Z₁, Z₂, Z₅ each are independently selected from H, F, Cl, Br, l, C₁-C₆ alkyl, C₁-C₆ alkoxy, C₁-C₆ fluorine-containing alkyl, or C₁-C₆ fluorine-containing alkoxy, and Z₁, Z₂, Z₅ may be hydrogen at the same time, R^{y} is C₁-C₆ alkyl;
11)

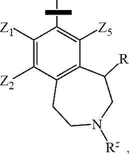

wherein Z₁, Z₂, Z₅ each are independently selected from H, or C₁-C₆ alkyl, and Z₁, Z₂, Z₅ may be hydrogen at the same time, R^{z}, R each are independently selected from C₁-C₆ alkyl.

In some embodiments, R¹ is selected from:
1) cyclopropyl, 2-N,N-dimethylaminoethyl, 2-hydroxyethyl, N-methyl-4-piperidyl, 4-aminocyclohexyl, 1-methyl-3-pyrazolyl, 1-ethyl-3-pyrazolyl, 1-isopropyl-3-pyrazolyl, 1,5-dimethyl-3-pyrazolyl, 1-methyl-4-pyrazolyl, 1-ethyl-4-pyrazolyl, 1-isopropyl-4-pyrazolyl, 1-ethyl-3-methyl-4-pyrazolyl, 1,3-dimethyl-5-pyrazolyl, 1-methyl-4-imidazolyl;
2)

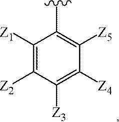

wherein:
   Z₁, Z₂, Z₃, Z₄, Z₅ each are independently selected from the following groups, and Z₁, Z₂, Z₃, Z₄, Z₅ are not hydrogen at the same time:
      H, F, 2-hydroxyethylamino, 2-N,N-dimethylaminoethylamino, N-methyl-N-(2-N,N-dimethylamino)ethylamino, methyl, ethyl, isopropyl, cyclopropyl, methoxy, ethoxy, isopropoxy, trifluoromethyl, trifluoromethoxy, vinyl, piperazin-1-yl, N-methylpiperazinyl, 4-(N-methylpiperidyl-4-)piperazinyl, 4-(azetidin-3-yl)piperazinyl, morpholinyl, 3-(N,N-dimethylamino)tetrahydropyrrolyl, 4-N,N-dimethylaminopiperidyl, 4-hydroxypiperidyl, 4-cyanopiperidyl, 4-(N-methylpiperazinyl-1-)piperidyl, 4-(piperazinyl-1 -)piperidyl, 4-(tetrahydropyrrolyl-1 -)piperidyl, N-methyl-4-piperidyl, 2-(N, N-dimethylam ino)acetam ido, cyclopropylsulfam ido, N-methyl-N-(2-N,N-dimethylamino)ethylamino, 4-methyl-1-piperazinylmethylene, benzyl, cyclopropylmethylene, 4-(N-methyl-1-piperazinyl)piperidyl-1-formyl, N-methylpiperazinyl-1-formyl, cyclopropylaminosulfonyl, N-methylpiperazinyl-1-sulfonyl, 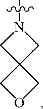 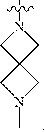 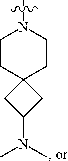 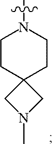
      preferably, Z₁, Z₂, Z₅ each are independently selected from H, F, methoxy, ethoxy, isopropoxy, trifluoromethyl, or trifluoromethoxy; and one of Z₃ and Z₄ is selected from the following groups and the other is H, F, methyl, ethyl, isopropyl, cyclopropyl, vinyl, benzyl or cyclopropylmethylene:
         F, 2-hydroxyethylamino, 2-N,N-dimethylaminoethylamino, N-methyl-N-(2-N,N-dimethylamino)ethylamino, piperazin-1-yl, N-methylpiperazinyl, 4-(N-methylpiperidyl-4-)piperazinyl, 4-(azetidin-3-yl)piperazinyl, morpholinyl, 3-(N,N-dimethylamino)tetrahydropyrrolyl, 4-N,N-dimethylaminopiperidyl, 4-hydroxypiperidyl, 4-cyanopiperidyl, 4-(N-methylpiperazinyl-1-)piperidyl, 4-(piperazinyl-1-)piperidyl, 4-(tetrahydropyrrolyl-1 -)piperidyl, N-methyl-4-piperidyl, 2-(N,N-dimethylamino)acetamido, cyclopropylsulfamido, N-methyl-N-(2-N,N-dimethylamino)ethylamino, 4-methyl-1-piperazinylmethylene, 4-(N-methyl-1-piperazinyl)piperidyl-1-formyl, N-methylpiperazinyl-1-formyl, cyclopropylaminosulfonyl, N-methylpiperazinyl-1-sulfonyl, 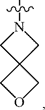 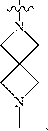 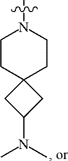 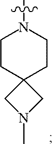
3)

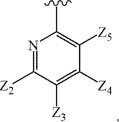

wherein Z₂, Z₃, Z₄, Z₅ each are independently selected from H, methoxy, or N-methylpiperazinyl, and Z₂, Z₃, Z₄, Z₅ are not hydrogen at the same time;
4)

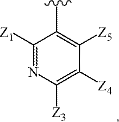

wherein:
   Z₁, Z₃, Z₄, Z₅ each are independently selected from H, methyl, ethyl, methoxy, N-(N-methyl-3-tetrahydropyrrolyl)amino, N-methyl-N-(N-methyl-3-tetrahydropyrrolyl)amino, N-methylpiperazinyl, or 4-(N-methylpiperazinyl-1-)piperidyl, and Z₁, Z₃, Z₄, Z₅ are not hydrogen at the same time;
   preferably, Z₁, Z₄, Z₅ each are independently selected from H, methyl, ethyl, or methoxy, Z₃ is methyl, N-(N-methyl-3-tetrahydropyrrolyl)amino, N-methyl-N-(N-methyl-3-tetrahydropyrrolyl)amino, N-methylpiperazinyl or 4-(N-methylpiperazinyl-1-)piperidyl;
5)

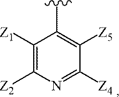

wherein Z₁, Z₂, Z₄, Z₅ are H;
6)

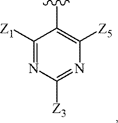

wherein Z₁, Z₃, Z₅ each are independently selected from H, methoxy, or N-methylpiperazinyl, and Z₁, Z₃, Z₅ are not hydrogen at the same time;
7)

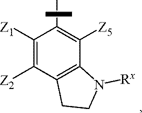

wherein Z₁, Z₂, Z₅ each are independently selected from H, or methoxy, R^{x} is 2-(N,N-dimethylamino)acetyl;
8)

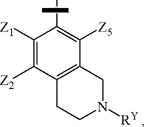

wherein Z₁, Z₂, Z₅ each are independently selected from H, F, Cl, methyl, methoxy, trifluoromethyl, or trifluoromethoxy, R^{y} is methyl;
9)

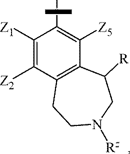

wherein Z₁, Z₂, Z₅ are H, R^{z}, R are methyl.

In some embodiments, R², R³ each are independently selected from H, C₁-C₆ alkyl, C₁-C₆ fluorine-containing alkyl, or C₃-C₆ cycloalkyl.

In some embodiments, R², R³ each are independently selected from H, methyl, trifluoromethyl, ethyl, isopropyl, or cyclopropyl.

In some embodiments, R² is selected from methyl, trifluoromethyl, ethyl, isopropyl, or cyclopropyl, R³ is selected from H, methyl, ethyl, isopropyl, or cyclopropyl.

In some embodiments, R⁵ is selected from H, F, Cl, Br, trifluoromethyl, cyano, nitro, methyl, ethyl, isopropyl, methoxy, ethoxy, or cyclopropyl.

In the third aspect of the present invention, the present invention provides a compound having the general formula (II-1) or (II-2):

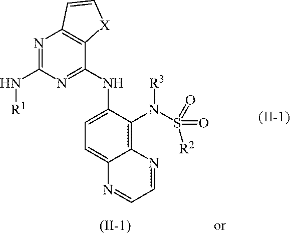

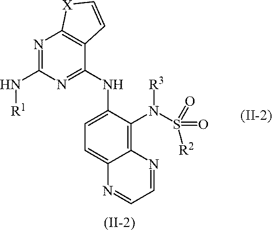

or a stereoisomer, a prodrug, a pharmaceutically acceptable salt or a pharmaceutically acceptable solvate thereof, wherein:
each X is independently selected from NH, O, or S;
each R¹ is independently selected from:
   1) 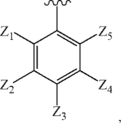
   wherein Z₁, Z₂, Z₃, Z₄, Z₅ each are independently selected from the following groups, and Z₁, Z₂, Z₃, Z₄, Z₅ are not hydrogen at the same time:
      (1) H, F, Cl, Br, I, nitro, cyano,
      (2) C₁-C₆ alkyl, C₁-C₆ alkoxy, C₁-C₆ oxygen-containing alkyl, C₁-C₆ fluorine-containing alkyl, C₁-C₆ fluorine-containing alkoxy, C₃-C₆ cycloalkyl, C₂-C₆ alkenyl,
      (3) 2-N,N-dimethylaminoethoxy, 3-N,N-dimethylaminopropoxy, 2-(N-methylpiperazinyl)ethoxy, 2-(N-acetylpiperazinyl)ethoxy, 2-morpholinylethoxy, 2-thiomorpholinylethoxy, 2-piperidylethoxy, 3-N,N-dimethylaminopropoxy, 3-N,N-diethylam inopropoxy, 3-N,N-diisopropylaminopropoxy, 3-(N-methylpiperazinyl)propoxy, 3-(N-acetylpiperazinyl)propoxy, 3-morpholinylpropoxy, 3-thiomorpholinylpropoxy, 3-piperidylpropoxy, 2-pyridylmethoxy, 3-pyridylmethoxy, 4-pyridylmethoxy, phenylmethoxy, monohalo-substituted phenylmethoxy, gem-dihalo-substituted phenylmethoxy, hetero-dihalo-substituted phenylmethoxy,
      (4) piperazinyl, N-methylpiperazinyl, N-ethylpiperazinyl, N-n-propylpiperazinyl, N-isopropylpiperazinyl, N-cyclopropylpiperazinyl, N-(2-hydroxyethyl)piperazinyl, N-(2-N,N-dimethylaminoethyl)piperazinyl, N-(2-N,N-diethylaminoethyl)piperazinyl, N-(2-cyanoethyl)piperazinyl, N-cyanomethylpiperazinyl, N-(3-hydroxypropyl)piperazinyl, N-(3-N,N-dimethylaminopropyl)piperazinyl, N-(3-N,N-diethylaminopropyl)piperazinyl, N-(N-methyl-4-piperidyl)piperazinyl, N-(N-ethyl-4-piperidyl)piperazinyl, N-t-butoxycarbonylpiperazinyl, N-acetylpiperazinyl, N-propionylpiperazinyl, N-isobutyrylpiperazinyl, N-cyclopropylformylpiperazinyl, N-methylsulfonylpiperazinyl, N-ethylsulfonylpiperazinyl, N-n-propylsulfonylpiperazinyl, N-isopropylsulfonylpiperazinyl, N-cyclopropylsulfonylpiperazinyl, 2-oxo-piperazin-4-yl,
      (5) morpholinyl, 2,6-dimethylmorpholinyl, thiomorpholinyl,
      (6) tetrahydropyrrolyl, 3-(N,N-dimethylamino)tetrahydropyrrolyl, 3-(N, N-diethylam ino)tetrahydropyrrolyl,
      (7) imidazolyl, 4-methyl-1-imidazolyl,
      (8) piperidyl, 4-N,N-dimethylaminopiperidyl, 4-N,N-diethylaminopiperidyl, 4-N,N-diisopropylaminopiperidyl, 4-hydroxypiperidyl, 4-methoxypiperidyl, 4-ethoxypiperidyl, 4-(N-methylpiperazinyl-1-)piperidyl, 4-(N-ethylpiperazinyl-1-)piperidyl, 4-(N-isopropylpiperazinyl-1-)piperidyl, 4-(N-acetylpiperazinyl-1-)piperidyl, 4-(N-t-butoxycarbonylpiperazinyl-1-)piperidyl, 4-(N-methylsulfonylpiperazinyl-1-)piperidyl, 4-(N-(2-hydroxyethyl)piperazinyl-1-)piperidyl, 4-(N-(2-cyanoethyl)piperazinyl-1-)piperidyl, 4-(N-(3-hydroxypropyl)piperazinyl-1-)piperidyl, 4-(N-(2-N, N-dimethylaminoethyl)piperazinyl-1-)piperidyl, 4-(N-(2-N,N-diethylethyl)piperazinyl-1-)piperidyl, 4-(N-(3-N,N-dimethylpropyl)piperazinyl-1-)piperidyl, 4-(N-(3-N,N-diethylpropyl)piperazinyl-1-)piperidyl, 4-(tetrahydropyrrolyl-1-)piperidyl, 4-(3-N, N-dimethylaminotetrahydropyrrolyl-1-)piperidyl, 4-(3-N, N-diethylam inotetrahydropyrrolyl-1-)piperidyl,
      (9) piperidin-4-yl, N-methyl-4-piperidyl, N-ethyl-4-piperidyl, N-isopropyl-4-piperidyl, N-acetyl-4-piperidyl, N-t-butoxycarbonyl-4-piperidyl, N-methylsulfonyl-4-piperidyl, N-(2-hydroxyethyl)-4-piperidyl, N-(2-N, N-dimethylaminoethyl)-4-piperidyl, N-(2-N, N-diethylam inoethyl)-4-piperidyl, N-(2-cyanoethyl)-4-piperidyl, N-(3-hydroxypropyl)-4-piperidyl, N-(3-N, N-dimethylaminopropyl)-4-piperidyl, N-(3-N, N-diethylam inopropyl)-4-piperidyl, N-(3-cyanopropyl)-4-piperidyl, N-cyanomethylene-4-piperidyl,
      (10) amino, N,N-dimethylamino, N,N-diethylamino, N,N-di-n-propylamino, N,N-diisopropylamino, 2-N,N-dimethylaminoethylamino, 2-morpholinylethylamino, 2-(N-methylpiperazinyl)ethylamino, 3-N, N-dimethylaminopropylamino, 3-N, N-diethylaminopropylamino, 3-N, N-diisopropylaminopropylamino, 3-morpholinylpropylamino, 3-(N-methylpiperazinyl)propylamino, N-methylpiperidyl-4-amino, N-ethylpiperidyl-4-amino, N-n-propylpiperidyl-4-amino, N-isopropylpiperidyl-4-amino, acetamido, propionamido, 2-(N,N-dimethylamino)acetamido, 2-hydroxyacetamido, 2-methoxyacetamido, methylsulfamido, ethylsulfamido, n-propylsulfamido, isopropylsulfamido, cyclopropylsulfamido, N-methyl-N-(N-methyl-3-tetrahydropyrrolyl)amino, N-(N-methyl-3-tetrahydropyrrolyl)amino, N-methyl-N-(2-N,N-dimethylamino)ethylamino,
      (11) aminomethylene, N,N-dimethylaminomethylene, N,N-diethylaminomethylene, 4-methyl-1-piperazinylmethylene, 4-ethyl-1-piperazinylmethylene, 4-propyl-1-piperazinylmethylene, 4-isopropyl-1-piperazinylmethylene, 4-(2-hydroxyethyl)-1-piperazinylmethylene, 4-(2-N,N-dimethylaminoethyl)-1-piperazinylmethylene, piperidyl-1-methylene, 4-(N,N-dimethylamino)-1-piperidylmethylene, 4-hydroxypiperidyl-1-methylene, tetrahydropyrrolyl-1-methylene, 3-(N,N-dimethylamino)-1-tetrahydropyrrolylmethylene, benzyl, cyclopropylmethylene, cyclobutylmethylene, cyclopentylmethylene, cyclohexylmethylene,
      (12) aminoformyl, methylaminoformyl, N,N-dimethylaminoformyl, ethylaminoformyl, cyclopropylaminoformyl, cyclobutylaminoformyl, cyclopentylaminoformyl, cyclohexylaminoformyl, aminoacetyl, methylaminoacetyl, 2-(N,N-dimethylamino)acetyl, ethylaminoacetyl, cyclopropylaminoacetyl, cyclobutylaminoacetyl, cyclopentylaminoacetyl, cyclohexylaminoacetyl, tetrahydropyrrolyl-1-formyl, 3-(N, N-dimethylamino)tetrahydropyrrolyl-1-formyl, 3-(N,N-diethylamino)tetrahydropyrrolyl-1-formyl, morpholinyl-4-formyl, thiomorpholinyl-4-formyl, 2,6-dimethylmorpholinyl-4-formyl, piperidyl-1-formyl, 4-hydroxypiperidyl-1-formyl, 4-(N,N-dimethylamino)piperidyl-1-formyl, 4-(N,N-diethylamino)piperidyl-1-formyl, 4-(N-methyl-1-piperazinyl)piperidyl-1-formyl, 4-(N-ethyl-1-piperazinyl)piperidyl-1-formyl, N-methylpiperazinyl-1-formyl, N-ethylpiperazinyl-1-formyl, N-isopropylpiperazinyl-1-formyl, N-(2-hydroxyethyl)piperazinyl-1-formyl, N-(2-N, N-dimethylaminoethyl)piperazinyl-1-formyl, N-(2-N, N-diethylaminoethyl)piperazinyl-1-formyl, 4-(N-methyl-4-piperidyl)piperazinyl-1-formyl, 4-(N-ethyl-4-piperidyl)piperazinyl-1-formyl,
      (13) hydroxyformyl, methoxyformyl, ethoxyformyl, propoxyformyl, isopropoxyformyl, n-butoxyformyl, iso-butoxyformyl, t-butoxyformyl,
      (14) hydroxysulfonyl, aminosulfonyl, methylaminosulfonyl, ethylaminosulfonyl, propylaminosulfonyl, isopropylaminosulfonyl, cyclopropylaminosulfonyl, cyclobutylaminosulfonyl, cyclopentylaminosulfonyl, cyclohexylaminosulfonyl, tetrahydropyrrolyl-1-sulfonyl, 3-(N,N-dimethylamino)tetrahydropyrrolyl-1-sulfonyl, 3-(N,N-diethylamino)tetrahydropyrrolyl-1-sulfonyl, morpholinyl-4-sulfonyl, thiomorpholinyl-4-sulfonyl, 2,6-dimethylmorpholinyl-4-sulfonyl, piperidyl-1-sulfonyl, 4-hydroxypiperidyl-1-sulfonyl, 4-(N,N-dimethylamino)piperidyl-1-sulfonyl, 4-(N,N-diethylamino)piperidyl-1-sulfonyl, 4-(N-methyl-1-piperazinyl)piperidyl-1-sulfonyl, 4-(N-ethyl-1-piperazinyl)piperidyl-1-sulfonyl, N-methylpiperazinyl-1-sulfonyl, N-ethylpiperazinyl-1-sulfonyl, N-isopropylpiperazinyl-1-sulfonyl, N-(2-hydroxyethyl)piperazinyl-1-sulfonyl, N-(2-N,N-dimethylaminoethyl)piperazinyl-1-sulfonyl, N-(2-N,N-diethylaminoethyl)piperazinyl-1-sulfonyl, 4-(N-methyl-4-piperidyl)piperazinyl-1-sulfonyl, 4-(N-ethyl-4-piperidyl)piperazinyl-1-sulfonyl,
      (15)aminoformamido,methylaminoformamido,ethylaminoformamido, propylaminoformamido,isopropylaminoformamido,cyclopropylaminoformamido, cyclobutylaminoformamido,cyclopentylaminoformamido,piperidyl-1-formamido, 4-hydroxypiperidyl-1-formamido,4-N,N-dimethylpiperidyl-1-formamido, 4-N,N-diethylpiperidyl-1-formamido, tetrahydropyrrolyl-1-formamido, 3-N,N-dimethyltetrahydropyrrolyl-1-formamido, 3-N,N-diethyltetrahydropyrrolyl-1-formamido,N-methylpiperazinyl-1-formamido, N-ethylpiperazinyl-1-formamido,N-acetylpiperazinyl-1-formamido, N-t-butoxycarbonylpiperazinyl-1-formamido,N-(2-hydroxyethyl)piperazinyl-1-formamido, N-(2-cyanoethyl)piperazinyl-1-formamido, N-(2-N,N-dimethylaminoethyl)piperazinyl-1-formamido, N-(2-N,N-diethylaminoethyl)piperazinyl-1-formamido, N-(3-hydroxypropyl)piperazinyl-1-formamido, N-(3-N,N-dimethylpropyl)piperazinyl-1-formamido, N-(3-N,N-diethylpropyl)piperazinyl-1-formamido,morpholinyl-1-formamido, 3,5-dimethylmorpholinyl-1-formamido,4-(tetrahydropyrrolyl)piperidyl-1-formamido, 4-(N-methyl-1-piperazinyl)piperidyl-1-formamido, 4-(N-ethyl-1-piperazinyl)piperidyl-1-formamido, 4-(N-acetyl-1-piperazinyl)piperidyl-1-formamido, N-(N-methyl-4-piperidyl)piperazinyl-1-formamido;
   2) 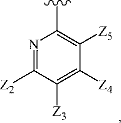
   wherein Z₂, Z₃, Z₄, Z₅ are the same as defined in 1), and Z₂, Z₃, Z₄, Z₅ are not hydrogen at the same time;
   3) 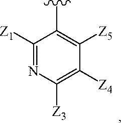
   wherein Z₁, Z₃, Z₄, Z₅ are the same as defined in 1), and Z₁, Z₃, Z₄, Z₅ are not hydrogen at the same time;
   4) 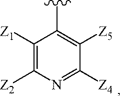
   wherein Z₁, Z₂, Z₄, Z₅ are the same as defined in 1), and Z₁, Z₂, Z₄, Z₅ may be hydrogen at the same time;
   5) 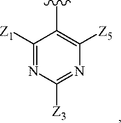
   wherein Z₁, Z₃, Z₅ are the same as defined in 1), and Z₁, Z₃, Z₅ are not hydrogen at the same time;
R², R³ each are independently selected from:
   H, C₁-C₆ alkyl, C₁-C₆ fluorine-containing alkyl, C₃-C₆ cycloalkyl, or C₃-C₆ fluorine-containing cycloalkyl.

In some embodiments, each R¹ is independently selected from:
1)

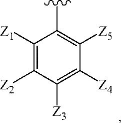

wherein Z₁, Z₂, Z₃, Z₄, Z₅ each are independently selected from the following groups, and Z₁, Z₂, Z₃, Z₄, Z₅ are not hydrogen at the same time:
   (1) H, F, Cl, Br, I, nitro, cyano,
   (2) C₁-C₆ alkyl, C₁-C₆ alkoxy, C₁-C₆ oxygen-containing alkyl, C₁-C₆ fluorine-containing alkyl, C₁-C₆ fluorine-containing alkoxy, C₃-C₆ cycloalkyl, C₂-C₆ alkenyl,
   (3) piperazinyl, N-methylpiperazinyl, N-ethylpiperazinyl, N-n-propylpiperazinyl, N-isopropylpiperazinyl, N-cyclopropylpiperazinyl, N-(2-hydroxyethyl)piperazinyl, N-(2-N,N-dimethylaminoethyl)piperazinyl, N-(2-N,N-diethylaminoethyl)piperazinyl, N-(2-cyanoethyl)piperazinyl, N-cyanomethylpiperazinyl, N-(3-hydroxypropyl)piperazinyl, N-(3-N,N-dimethylaminopropyl)piperazinyl, N-(3-N,N-diethylaminopropyl)piperazinyl,
   (4) morpholinyl, 2,6-dimethylmorpholinyl, thiomorpholinyl,
   (5) tetrahydropyrrolyl, 3-(N,N-dimethylamino)tetrahydropyrrolyl, 3-(N, N-diethylam ino)tetrahydropyrrolyl,
   (6) imidazolyl, 4-methyl-1-imidazolyl,
   (7) piperidyl, 4-N,N-dimethylaminopiperidyl, 4-N,N-diethylaminopiperidyl, 4-N,N-diisopropylaminopiperidyl, 4-hydroxypiperidyl, 4-methoxypiperidyl, 4-ethoxypiperidyl, 4-(N-methylpiperazinyl-1-)piperidyl, 4-(N-ethylpiperazinyl-1-)piperidyl, 4-(N-isopropylpiperazinyl-1-)piperidyl, 4-(N-(2-hydroxyethyl)piperazinyl-1-)piperidyl, 4-(N-(2-cyanoethyl)piperazinyl-1-)piperidyl, 4-(N-(3-hydroxypropyl)piperazinyl-1-)piperidyl, 4-(N-(2-N,N-dimethylaminoethyl)piperazinyl-1-)piperidyl, 4-(N-(2-N,N-diethylethyl)piperazinyl-1-)piperidyl, 4-(N-(3-N,N-dimethylpropyl)piperazinyl-1-)piperidyl, 4-(N-(3-N,N-diethylpropyl)piperazinyl-1-)piperidyl, 4-(tetrahydropyrrolyl-1-)piperidyl, 4-(3-N,N-dimethylaminotetrahydropyrrolyl-1-)piperidyl, 4-(3-N,N-diethylaminotetrahydropyrrolyl-1-)piperidyl,
   (8) piperidin-4-yl, N-methyl-4-piperidyl, N-ethyl-4-piperidyl, N-isopropyl-4-piperidyl, N-(2-hydroxyethyl)-4-piperidyl, N-(2-N,N-dimethylaminoethyl)-4-piperidyl, N-(2-N,N-diethylaminoethyl)-4-piperidyl, N-(2-cyanoethyl)-4-piperidyl, N-(3-hydroxypropyl)-4-piperidyl, N-(3-N,N-dimethylaminopropyl)-4-piperidyl, N-(3-N,N-diethylaminopropyl)-4-piperidyl, N-(3-cyanopropyl)-4-piperidyl, N-cyanomethylene-4-piperidyl,
   (9) amino, N,N-dimethylamino, N,N-diethylamino, N,N-di-n-propylamino, N,N-diisopropylamino, 2-N,N-dimethylaminoethylamino, 3-N,N-dimethylaminopropylamino, 3-N, N-diethylaminopropylamino, 3-N,N-diisopropylaminopropylamino, N-methylpiperidyl-4-amino, N-ethylpiperidyl-4-amino, N-n-propylpiperidyl-4-amino, N-isopropylpiperidyl-4-amino, acetamido, propionamido, 2-(N,N-dimethylamino)acetamido, 2-hydroxyacetamido, 2-methoxyacetamido, methylsulfamido, ethylsulfamido, n-propylsulfamido, isopropylsulfamido, cyclopropylsulfamido, N-methyl-N-(N-methyl-3-tetrahydropyrrolyl)amino, N-(N-methyl-3-tetrahydropyrrolyl)amino, N-methyl-N-(2-N,N-dimethylamino)ethylamino,
   (10) 4-methyl-1-piperazinylmethylene, 4-ethyl-1-piperazinylmethylene, 4-propyl-1-piperazinylmethylene, 4-isopropyl-1-piperazinylmethylene, 4-(2-hydroxyethyl)-1-piperazinylmethylene, 4-(2-N,N-dimethylaminoethyl)-1-piperazinylmethylene, piperidyl-1-methylene, 4-(N,N-dimethylamino)-1-piperidylmethylene, 4-hydroxypiperidyl-1-methylene, tetrahydropyrrolyl-1-methylene, 3-(N,N-dimethylamino)-1-tetrahydropyrrolylmethylene, benzyl, cyclopropylmethylene, cyclobutylmethylene, cyclopentylmethylene, cyclohexylmethylene,
   (11) aminoformyl, methylaminoformyl, N,N-dimethylaminoformyl, ethylaminoformyl, aminoacetyl, methylaminoacetyl, 2-(N,N-dimethylamino)acetyl, ethylaminoacetyl, tetrahydropyrrolyl-1-formyl, 3-(N, N-dimethylamino)tetrahydropyrrolyl-1-formyl, 3-(N,N-diethylamino)tetrahydropyrrolyl-1-formyl, morpholinyl-4-formyl, thiomorpholinyl-4-formyl, 2,6-dimethylmorpholinyl-4-formyl, piperidyl-1-formyl, 4-hydroxypiperidyl-1-formyl, 4-(N,N-dimethylamino)piperidyl-1-formyl, 4-(N,N-diethylamino)piperidyl-1-formyl, 4-(N-methyl-1-piperazinyl)piperidyl-1-formyl, 4-(N-ethyl-1-piperazinyl)piperidyl-1-formyl, N-methylpiperazinyl-1-formyl, N-ethylpiperazinyl-1-formyl, N-isopropylpiperazinyl-1-formyl, N-(2-hydroxyethyl)piperazinyl-1-formyl, N-(2-N,N-dimethylaminoethyl)piperazinyl-1-formyl, N-(2-N,N-diethylaminoethyl)piperazinyl-1-formyl, 4-(N-methyl-4-piperidyl)piperazinyl-1-formyl, 4-(N-ethyl-4-piperidyl)piperazinyl-1-formyl,
   (12) hydroxysulfonyl, aminosulfonyl, methylaminosulfonyl, ethylaminosulfonyl, propylaminosulfonyl, isopropylaminosulfonyl, cyclopropylaminosulfonyl, cyclobutylaminosulfonyl, cyclopentylaminosulfonyl, cyclohexylaminosulfonyl, tetrahydropyrrolyl-1-sulfonyl, 3-(N,N-dimethylamino)tetrahydropyrrolyl-1-sulfonyl, 3-(N,N-diethylamino)tetrahydropyrrolyl-1-sulfonyl, morpholinyl-4-sulfonyl, thiomorpholinyl-4-sulfonyl, 2,6-dimethylmorpholinyl-4-sulfonyl, piperidyl-1-sulfonyl, 4-hydroxypiperidyl-1-sulfonyl, 4-(N,N-dimethylamino)piperidyl-1-sulfonyl, 4-(N,N-diethylamino)piperidyl-1-sulfonyl, N-methylpiperazinyl-1-sulfonyl, N-ethylpiperazinyl-1-sulfonyl, N-isopropylpiperazinyl-1-sulfonyl, N-(2-hydroxyethyl)piperazinyl-1-sulfonyl, N-(2-N,N-dimethylaminoethyl)piperazinyl-1-sulfonyl, N-(2-N,N-diethylam inoethyl)piperazinyl-1-sulfonyl,
   (13) aminoformamido, methylaminoformamido, ethylaminoformamido, propylaminoformamido, isopropylaminoformamido;
2)

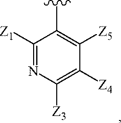

wherein Z₁, Z₃, Z₄, Z₅ are the same as defined in 1), and Z₁, Z₃, Z₄, Z₅ are not hydrogen at the same time.

In some embodiments, each R¹ is independently selected from:
1)

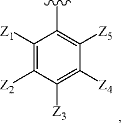

wherein Z₁, Z₂, Z₃, Z₄, Z₅ each are independently selected from the following groups, and Z₁, Z₂, Z₃, Z₄, Z₅ are not hydrogen at the same time:
   (1) H, F, Cl, Br, I, nitro, cyano,
   (2) C₁-C₆ alkyl, C₁-C₆ alkoxy, C₁-C₆ fluorine-containing alkoxy, C₂-C₆ alkenyl,
   (3) piperazinyl, N-methylpiperazinyl, N-ethylpiperazinyl, N-n-propylpiperazinyl, N-isopropylpiperazinyl,
   (5) 3-(N,N-dimethylamino)tetrahydropyrrolyl, 3-(N,N-diethylamino)tetrahydropyrrolyl,
   (6) 4-N,N-dimethylaminopiperidyl, 4-N,N-diethylaminopiperidyl, 4-N,N-diisopropylaminopiperidyl, 4-(N-methylpiperazinyl-1-)piperidyl, 4-(N-ethylpiperazinyl-1-)piperidyl, 4-(N-isopropylpiperazinyl-1-)piperidyl, 4-hydroxypiperidyl,
   (7) benzyl, cyclopropylmethylene, cyclobutylmethylene, cyclopentylmethylene, cyclohexylmethylene,
2)

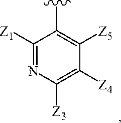

wherein Z₁, Z₃, Z₄, Z₅ each are independently selected from H, C₁-C₆ alkyl, C₁-C₆ alkoxy, 4-(N-methylpiperazinyl-1-)piperidyl, 4-(N-ethylpiperazinyl-1-)piperidyl, or 4-(N-isopropylpiperazinyl-1-)piperidyl, and Z₁, Z₃, Z₄, Z₅ are not hydrogen at the same time.

In some embodiments, each R¹ is independently selected from:
1)

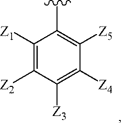

wherein:
   Z₁, Z₂, Z₃, Z₄, Z₅ each are independently selected from the following groups, and Z₁, Z₂, Z₃, Z₄, Z₅ are not hydrogen at the same time:
      H, methyl, ethyl, methoxy, trifluoromethoxy, vinyl, N-methylpiperazinyl, 3-(N,N-dimethylamino)tetrahydropyrrolyl, 4-N,N-dimethylaminopiperidyl, 4-(N-methylpiperazinyl-1-)piperidyl, 4-hydroxypiperidyl, benzyl, or cyclopropylmethylene preferably, Z₁, Z₂, Z₅ each are independently selected from H, methoxy, or trifluoromethoxy, Z₄ is H, methyl, ethyl, vinyl, benzyl, cyclopropylmethylene or 4-hydroxypiperidyl, Z₃ is H, N-methylpiperazinyl, 3-(N,N-dimethylamino)tetrahydropyrrolyl, 4-N,N-dimethylamino piperidyl or 4-(N-methylpiperazinyl-1-)piperidyl, Z₁, Z₂, Z₃, Z₄, Z₅ are not hydrogen at the same time;
2)

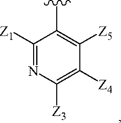

wherein:
   Z₁, Z₃, Z₄, Z₅ each are independently selected from H, methyl, ethyl, methoxy, or 4-(N-methylpiperazinyl-1-)piperidyl, and Z₁, Z₃, Z₄, Z₅ are not hydrogen at the same time;
   preferably, Z₁, Z₄, Z₅ each are independently selected from H, methyl, ethyl, or methoxy, Z₃ is 4-(N-methylpiperazinyl-1-)piperidyl.

In some embodiments, R², R³ each are independently selected from H, or C₁-C₆ alkyl.

In some embodiments, R², R³ each are independently selected from methyl.

In the fourth aspect of the present invention, the present invention provides a compound having the general formula (A):

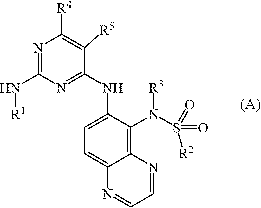

or a stereoisomer, a prodrug, a pharmaceutically acceptable salt or a pharmaceutically acceptable solvate thereof, wherein:
R¹ is selected from:
   1) H, C₁-C₆ alkyl, C₁-C₆ fluorine-containing alkyl, C₃-C₈ cycloalkyl, C₁-C₆ heteroatom-containing alkyl, C₃-C₈ heteroatom-containing cycloalkyl;
   2) 2-N,N-dimethylaminoethyl, 3-N,N-dimethylaminopropyl, 2-N,N-diethylaminoethyl, 3-N,N-diethylaminopropyl, 2-(N-methylpiperazinyl)ethyl, 2-(N-acetylpiperazinyl)ethyl, 2-morpholinylethyl, 2-thiomorpholinylethyl, 2-piperidylethyl, 2-hydroxyethyl, 3-hydroxypropyl, 2-methoxyethyl, N-methyl-4-piperidyl, N-ethyl-4-piperidyl, N-propyl-4-piperidyl, N-isopropyl-4-piperidyl, N-hydroxyethyl-4-piperidyl, N-cyanomethyl-4-piperidyl, N-(2-cyanoethyl)-4-piperidyl, N-acetyl-4-piperidyl, N-propionyl-4-piperidyl, N-isopropionyl-4-piperidyl, N-cyclopropylformyl-4-piperidyl, N-methylsulfonyl-4-piperidyl, N-ethylsulfonyl-4-piperidyl, N-propylsulfonyl-4-piperidyl, N-isopropylsulfonyl-4-piperidyl, N-cyclopropylsulfonyl-4-piperidyl, 3-N,N-dimethylaminocyclopentyl, 3-N,N-diethylam inocyclopentyl, 3-N,N-dipropylaminocyclopentyl, 3-N,N-isopropylaminocyclopentyl, 4-aminocyclohexyl, 4-N,N-dimethylaminocyclohexyl, 4-N,N-diethylam inocyclohexyl, 4-N,N-dipropylaminocyclohexyl, 4-N,N-diisopropylaminocyclohexyl, 4-acetylaminocyclohexyl, 4-propionylaminocyclohexyl, 4-isopropylformylaminocyclohexyl, 4-methylsulfonylaminocyclohexyl, 4-ethylsulfonylaminocyclohexyl, 4-propylsulfonylaminocyclohexyl, 4-isopropylsulfonylaminocyclohexyl, 4-cyclopropylsulfonylaminocyclohexyl, 4-hydroxycyclohexyl, 4-methoxycyclohexyl, 4-ethoxycyclohexyl, 4-isopropoxycyclohexyl;
   3) 1-methyl-3-pyrrolyl, 1-ethyl-3-pyrrolyl, 1-isopropyl-3-pyrrolyl, 1-cyclopropyl-3-pyrrolyl, 1-methyl-3-pyrazolyl, 1-ethyl-3-pyrazolyl, 1-isopropyl-3-pyrazolyl, 1-cyclopropyl-3-pyrazolyl, 1-hydroxyethyl-3-pyrazolyl, 1-methyl-4-pyrazolyl, 1-ethyl-4-pyrazolyl, 1-isopropyl-4-pyrazolyl, 1-cyclopropyl-4-pyrazolyl, 1-hydroxyethyl-4-pyrazolyl, 1-(N-methyl-4-piperidyl)-4-pyrazolyl, 1-(N-t-butoxycarbonyl-4-piperidyl)-4-pyrazolyl, 1,3-dimethyl-5-pyrazolyl, 1-methyl-4-imidazolyl, 3-methyl-5-isoxazolyl;
   4) 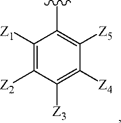
   wherein Z₁, Z₂, Z₃, Z₄, Z₅ each are independently selected from the following groups, and Z₁, Z₂, Z₃, Z₄, Z₅ are not hydrogen at the same time:
      (1) H, F, Cl, Br, I, nitro, cyano,
      (2) C₁-C₆ alkyl, C₁-C₆ alkoxy, C₁-C₆ oxygen-containing alkyl, C₁-C₆ fluorine-containing alkyl, C₁-C₆ fluorine-containing alkoxy, C₃-C₆ cycloalkyl, C₂-C₆ alkenyl,
      (3) 2-N,N-dimethylaminoethoxy, 3-N,N-dimethylaminopropoxy, 2-(N-methylpiperazinyl)ethoxy, 2-(N-acetylpiperazinyl)ethoxy, 2-morpholinylethoxy, 2-thiomorpholinylethoxy, 2-piperidylethoxy, 3-N,N-dimethylaminopropoxy, 3-N,N-diethylaminopropoxy, 3-N,N-diisopropylaminopropoxy, 3-(N-methylpiperazinyl)propoxy, 3-(N-acetylpiperazinyl)propoxy, 3-morpholinylpropoxy, 3-thiomorpholinylpropoxy, 3-piperidylpropoxy, 2-pyridylmethoxy, 3-pyridylmethoxy, 4-pyridylmethoxy, phenylmethoxy, monohalo-substituted phenylmethoxy, gem-dihalo-substituted phenylmethoxy, hetero-dihalo-substituted phenylmethoxy,
      (4) piperazinyl, N-methylpiperazinyl, N-ethylpiperazinyl, N-n-propylpiperazinyl, N-isopropylpiperazinyl, N-cyclopropylpiperazinyl, N-(2-hydroxyethyl)piperazinyl, N-(2-N,N-dimethylaminoethyl)piperazinyl, N-(2-N,N-diethylaminoethyl)piperazinyl, N-(2-cyanoethyl)piperazinyl, N-cyanomethylpiperazinyl, N-(3-hydroxypropyl)piperazinyl, N-(3-N,N-dimethylaminopropyl)piperazinyl, N-(3-N,N-diethylaminopropyl)piperazinyl, N-(N-methyl-4-piperidyl)piperazinyl, N-(N-ethyl-4-piperidyl)piperazinyl, N-t-butoxycarbonylpiperazinyl, N-acetylpiperazinyl, N-propionylpiperazinyl, N-isobutyrylpiperazinyl, N-cyclopropylformylpiperazinyl, N-methylsulfonylpiperazinyl, N-ethylsulfonylpiperazinyl, N-n-propylsulfonylpiperazinyl, N-isopropylsulfonylpiperazinyl, N-cyclopropylsulfonylpiperazinyl, 2-oxo-piperazin-4-yl,
      (5) morpholinyl, 2,6-dimethylmorpholinyl, thiomorpholinyl,
      (6) tetrahydropyrrolyl, 3-(N,N-dimethylamino)tetrahydropyrrolyl, 3-(N, N-diethylam ino)tetrahydropyrrolyl,
      (7) imidazolyl, 4-methyl-1-imidazolyl,
      (8) piperidyl, 4-N,N-dimethylaminopiperidyl, 4-N,N-diethylaminopiperidyl, 4-N,N-diisopropylaminopiperidyl, 4-hydroxypiperidyl, 4-methoxypiperidyl, 4-ethoxypiperidyl, 4-(N-methylpiperazinyl-1-)piperidyl, 4-(N-ethylpiperazinyl-1-)piperidyl, 4-(N-isopropylpiperazinyl-1-)piperidyl, 4-(N-acetylpiperazinyl-1-)piperidyl, 4-(N-t-butoxycarbonylpiperazinyl-1-)piperidyl, 4-(N-methylsulfonylpiperazinyl-1-)piperidyl, 4-(N-(2-hydroxyethyl)piperazinyl-1-)piperidyl, 4-(N-(2-cyanoethyl)piperazinyl-1-)piperidyl, 4-(N-(3-hydroxypropyl)piperazinyl-1-)piperidyl, 4-(N-(2-N, N-dimethylaminoethyl)piperazinyl-1-)piperidyl, 4-(N-(2-N,N-diethylethyl)piperazinyl-1 -)piperidyl, 4-(N-(3-N,N-dimethylpropyl)piperazinyl-1-)piperidyl, 4-(N-(3-N,N-diethylpropyl)piperazinyl-1-)piperidyl, 4-(tetrahydropyrrolyl-1-)piperidyl, 4-(3-N,N-dimethylaminotetrahydropyrrolyl-1-)piperidyl, 4-(3-N,N-diethylam inotetrahydropyrrolyl-1-)piperidyl,
      (9) piperidin-4-yl, N-methyl-4-piperidyl, N-ethyl-4-piperidyl, N-isopropyl-4-piperidyl, N-acetyl-4-piperidyl, N-t-butoxycarbonyl-4-piperidyl, N-methylsulfonyl-4-piperidyl, N-(2-hydroxyethyl)-4-piperidyl, N-(2-N,N-dimethylaminoethyl)-4-piperidyl, N-(2-N, N-diethylam inoethyl)-4-piperidyl, N-(2-cyanoethyl)-4-piperidyl, N-(3-hydroxypropyl)-4-piperidyl, N-(3-N,N-dimethylaminopropyl)-4-piperidyl, N-(3-N, N-diethylam inopropyl)-4-piperidyl, N-(3-cyanopropyl)-4-piperidyl, N-cyanomethylene-4-piperidyl,
      (10) amino, N,N-dimethylamino, N,N-diethylamino, N,N-di-n-propylamino, N,N-diisopropylamino, 2-N,N-dimethylaminoethylamino, 2-morpholinylethylamino, 2-(N-methylpiperazinyl)ethylamino, 3-N, N-dimethylaminopropylamino, 3-N, N-diethylam inopropylamino, 3-N,N-diisopropylaminopropylamino, 3-morpholinylpropylamino, 3-(N-methylpiperazinyl)propylamino, N-methylpiperidyl-4-amino, N-ethylpiperidyl-4-amino, N-n-propylpiperidyl-4-amino, N-isopropylpiperidyl-4-amino, acetamido, propionamido, 2-(N,N-dimethylamino)acetamido, 2-hydroxyacetamido, 2-methoxyacetamido, methylsulfamido, ethylsulfamido, n-propylsulfamido, isopropylsulfamido, cyclopropylsulfamido, N-methyl-N-(N-methyl-3-tetrahydropyrrolyl)amino, N-(N-methyl-3-tetrahydropyrrolyl)amino, N-methyl-N-(2-N,N-dimethylamino)ethylamino,
      (11) aminomethylene, N,N-dimethylaminomethylene, N,N-diethylaminomethylene, 4-methyl-1-piperazinylmethylene, 4-ethyl-1-piperazinylmethylene, 4-propyl-1-piperazinylmethylene, 4-isopropyl-1-piperazinylmethylene, 4-(2-hydroxyethyl)-1-piperazinylmethylene, 4-(2-N,N-dimethylaminoethyl)-1-piperazinylmethylene, piperidyl-1-methylene, 4-(N,N-dimethylamino)-1-piperidylmethylene, 4-hydroxypiperidyl-1-methylene, tetrahydropyrrolyl-1-methylene, 3-(N,N-dimethylamino)-1-tetrahydropyrrolylmethylene, benzyl, cyclopropylmethylene, cyclobutylmethylene, cyclopentylmethylene, cyclohexylmethylene,
      (12) aminoformyl, methylaminoformyl, N,N-dimethylaminoformyl, ethylaminoformyl, cyclopropylaminoformyl, cyclobutylaminoformyl, cyclopentylaminoformyl, cyclohexylaminoformyl, aminoacetyl, methylaminoacetyl, 2-(N,N-dimethylamino)acetyl, ethylaminoacetyl, cyclopropylaminoacetyl, cyclobutylaminoacetyl, cyclopentylaminoacetyl, cyclohexylaminoacetyl, tetrahydropyrrolyl-1-formyl, 3-(N,N-dimethylamino)tetrahydropyrrolyl-1-formyl, 3-(N,N-diethylamino)tetrahydropyrrolyl-1-formyl, morpholinyl-4-formyl, thiomorpholinyl-4-formyl, 2,6-dimethylmorpholinyl-4-formyl, piperidyl-1-formyl, 4-hydroxypiperidyl-1-formyl, 4-(N,N-dimethylamino)piperidyl-1-formyl, 4-(N,N-diethylamino)piperidyl-1-formyl, 4-(N-methyl-1-piperazinyl)piperidyl-1-formyl, 4-(N-ethyl-1-piperazinyl)piperidyl-1-formyl, N-methylpiperazinyl-1-formyl, N-ethylpiperazinyl-1-formyl, N-isopropylpiperazinyl-1-formyl, N-(2-hydroxyethyl)piperazinyl-1-formyl, N-(2-N,N-dimethylaminoethyl)piperazinyl-1-formyl, N-(2-N,N-diethylaminoethyl)piperazinyl-1-formyl, 4-(N-methyl-4-piperidyl)piperazinyl-1-formyl, 4-(N-ethyl-4-piperidyl)piperazinyl-1-formyl,
      (13) hydroxyformyl, methoxyformyl, ethoxyformyl, propoxyformyl, isopropoxyformyl, n-butoxyformyl, iso-butoxyformyl, t-butoxyformyl,
      (14) hydroxysulfonyl, aminosulfonyl, methylaminosulfonyl, ethylaminosulfonyl, propylaminosulfonyl, isopropylaminosulfonyl, cyclopropylaminosulfonyl, cyclobutylaminosulfonyl, cyclopentylaminosulfonyl, cyclohexylaminosulfonyl, tetrahydropyrrolyl-1-sulfonyl, 3-(N,N-dimethylamino)tetrahydropyrrolyl-1-sulfonyl, 3-(N,N-diethylamino)tetrahydropyrrolyl-1-sulfonyl, morpholinyl-4-sulfonyl, thiomorpholinyl-4-sulfonyl, 2,6-dimethylmorpholinyl-4-sulfonyl, piperidyl-1-sulfonyl, 4-hydroxypiperidyl-1-sulfonyl, 4-(N,N-dimethylamino)piperidyl-1-sulfonyl, 4-(N,N-diethylamino)piperidyl-1-sulfonyl, 4-(N-methyl-1-piperazinyl)piperidyl-1-sulfonyl, 4-(N-ethyl-1-piperazinyl)piperidyl-1-sulfonyl, N-methylpiperazinyl-1-sulfonyl, N-ethylpiperazinyl-1-sulfonyl, N-isopropylpiperazinyl-1-sulfonyl, N-(2-hydroxyethyl)piperazinyl-1-sulfonyl, N-(2-N,N-dimethylaminoethyl)piperazinyl-1-sulfonyl, N-(2-N,N-diethylaminoethyl)piperazinyl-1-sulfonyl, 4-(N-methyl-4-piperidyl)piperazinyl-1-sulfonyl, 4-(N-ethyl-4-piperidyl)piperazinyl-1-sulfonyl,
      (15) aminoformamido, methylaminoformamido, ethylaminoformamido, propylaminoformamido, isopropylaminoformamido, cyclopropylaminoformamido, cyclobutylaminoformamido, cyclopentylaminoformamido, piperidyl-1-formamido, 4-hydroxypiperidyl-1-formamido, 4-N,N-dimethylpiperidyl-1-formamido, 4-N,N-diethylpiperidyl-1-formamido, tetrahydropyrrolyl-1-formamido, 3-N,N-dimethyltetrahydropyrrolyl-1-formamido, 3-N,N-diethyltetrahydropyrrolyl-1-formamido, N-methylpiperazinyl-1-formamido, N-ethylpiperazinyl-1-formamido, N-acetylpiperazinyl-1-formamido, N-t-butoxycarbonylpiperazinyl-1-formamido, N-(2-hydroxyethyl)piperazinyl-1-formamido, N-(2-cyanoethyl)piperazinyl-1-formamido, N-(2-N,N-dimethylaminoethyl)piperazinyl-1-formamido, N-(2-N,N-diethylaminoethyl)piperazinyl-1-formamido, N-(3-hydroxypropyl)piperazinyl-1-formamido, N-(3-N,N-dimethylpropyl)piperazinyl-1 -formamido, N-(3-N,N-diethylpropyl)piperazinyl-1-formamido, morpholinyl-1-formamido, 3,5-dimethylmorpholinyl-1-formamido, 4-(tetrahydropyrrolyl) piperidyl-1-formamido, 4-(N-methyl-1-piperazinyl)piperidyl-1-formamido, 4-(N-ethyl-1-piperazinyl)piperidyl-1-formamido, 4-(N-acetyl-1-piperazinyl)piperidyl-1-formamido, N-(N-methyl-4-piperidyl)piperazinyl-1-formamido,
      (16) Z₃ and Z₄ may form an oxygen-containing substituted or unsubstituted 5- to 7-membered ring; the substituent may be selected from the same substituents as described above for Z₁,
      (17) Z₃ and Z₄ may form a nitrogen-containing substituted or unsubstituted 5- to 7-membered ring; the substituent may be selected from the same substituents as described above for Z₁,
   5) 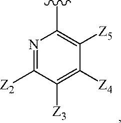
   wherein Z₂, Z₃, Z₄, Z₅ are the same as defined in 4), and Z₂, Z₃, Z₄, Z₅ are not hydrogen at the same time;
   6) 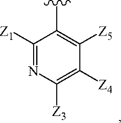
   wherein Z₁, Z₃, Z₄, Z₅ are the same as defined in 4), and Z₁, Z₃, Z₄, Z₅ are not hydrogen at the same time;
   7) 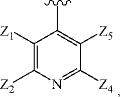
   wherein Z₁, Z₂, Z₄, Z₅ are the same as defined in 4), and Z₁, Z₂, Z₄, Z₅ may be hydrogen at the same time;
   8) 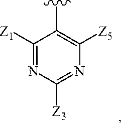
   wherein Z₁, Z₃, Z₅ are the same as defined in 4), and Z₁, Z₃, Z₅ are not hydrogen at the same time;
R², R³ each are independently selected from:
   H, C₁—C₆ alkyl, C₁—C₆ fluorine-containing alkyl, C₃—C₆ cycloalkyl, or C₃—C₆ fluorine-containing cycloalkyl;
   R⁴, R⁵ each are independently selected from:
      H, F, Cl, Br, C₁—C₆ fluorine-containing alkyl (e.g., trifluoromethyl), cyano, nitro, C₁—C₆ alkyl (e.g., methyl), C₁—C₆ alkoxy (e.g., methoxy), or C₃—C₆ cycloalkyl (e.g., cyclopropyl), or, R⁴, R⁵ and carbon atoms linked thereto together form a N—, O— or S-containing 5-membered ring.

In some embodiments, R¹ is selected from:
1) H, C₁—C₆ alkyl, C₁—C₆ fluorine-containing alkyl, C₃—C₈ cycloalkyl, C₁—C₆ heteroatom-containing alkyl, C₃—C₈ heteroatom-containing cycloalkyl;
2) 2-N,N-dimethylaminoethyl, 3-N,N-dimethylaminopropyl, 2-N,N-diethylaminoethyl, 3-N,N-diethylaminopropyl, 2-(N-methylpiperazinyl)ethyl, 2-(N-acetylpiperazinyl)ethyl, 2-morpholinylethyl, 2-thiomorpholinylethyl, 2-piperidylethyl, 2-hydroxyethyl, 3-hydroxypropyl, 2-methoxyethyl, N-methyl-4-piperidyl, N-ethyl-4-piperidyl, N-propyl-4-piperidyl, N-isopropyl-4-piperidyl, N-hydroxyethyl-4-piperidyl, N-cyanomethyl-4-piperidyl, N-(2-cyanoethyl)-4-piperidyl, 3-N,N-dimethylaminocyclopentyl, 3-N,N-diethylaminocyclopentyl, 3-N,N-dipropylaminocyclopentyl, 3-N,N-isopropylaminocyclopentyl, 4-aminocyclohexyl, 4-N,N-dimethylaminocyclohexyl, 4-N,N-diethylaminocyclohexyl, 4-N,N-dipropylaminocyclohexyl, 4-N,N-diisopropylaminocyclohexyl, 4-hydroxycyclohexyl, 4-methoxycyclohexyl, 4-ethoxycyclohexyl, 4-isopropoxycyclohexyl;
3) 1-methyl-3-pyrrolyl, 1-ethyl-3-pyrrolyl, 1-isopropyl-3-pyrrolyl, 1-cyclopropyl-3-pyrrolyl, 1-methyl-3-pyrazolyl, 1-ethyl-3-pyrazolyl, 1-isopropyl-3-pyrazolyl, 1-cyclopropyl-3-pyrazolyl, 1-hydroxyethyl-3-pyrazolyl, 1-methyl-4-pyrazolyl, 1-ethyl-4-pyrazolyl, 1-isopropyl-4-pyrazolyl, 1-cyclopropyl-4-pyrazolyl, 1-hydroxyethyl-4-pyrazolyl, 1,3-dimethyl-5-pyrazolyl, 1-methyl-4-imidazolyl, 3-methyl-5-isoxazolyl;
4)

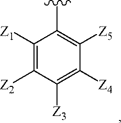

wherein Z₁, Z₂, Z₃, Z₄, Z₅ each are independently selected from the following groups, and Z₁, Z₂, Z₃, Z₄, Z₅ are not hydrogen at the same time:
   (1) H, F, Cl, Br, I, nitro, cyano,
   (2) C₁-C₆ alkyl, C₁-C₆ alkoxy, C₁-C₆ oxygen-containing alkyl, C₁-C₆ fluorine-containing alkyl, C₁-C₆ fluorine-containing alkoxy, C₃-C₆ cycloalkyl, C₂-C₆ alkenyl,
   (3) piperazinyl, N-methylpiperazinyl, N-ethylpiperazinyl, N-n-propylpiperazinyl, N-isopropylpiperazinyl, N-cyclopropylpiperazinyl, N-(2-hydroxyethyl)piperazinyl, N-(2-N,N-dimethylaminoethyl)piperazinyl, N-(2-N,N-diethylaminoethyl)piperazinyl, N-(2-cyanoethyl)piperazinyl, N-cyanomethylpiperazinyl, N-(3-hydroxypropyl)piperazinyl, N-(3-N,N-dimethylaminopropyl)piperazinyl, N-(3-N,N-diethylaminopropyl)piperazinyl,
   (4) morpholinyl, 2,6-dimethylmorpholinyl, thiomorpholinyl,
   (5) tetrahydropyrrolyl, 3-(N,N-dimethylamino)tetrahydropyrrolyl, 3-(N,N-diethylamino)tetrahydropyrrolyl,
   (6) imidazolyl, 4-methyl-1-imidazolyl,
   (7) piperidyl, 4-N,N-dimethylaminopiperidyl, 4-N,N-diethylaminopiperidyl, 4-N,N-diisopropylaminopiperidyl, 4-hydroxypiperidyl, 4-methoxypiperidyl, 4-ethoxypiperidyl, 4-(N-methylpiperazinyl-1-)piperidyl, 4-(N-ethylpiperazinyl-1-)piperidyl, 4-(N-isopropylpiperazinyl-1-)piperidyl, 4-(N-(2-hydroxyethyl)piperazinyl-1-)piperidyl, 4-(N-(2-cyanoethyl)piperazinyl-1-)piperidyl, 4-(N-(3-hydroxypropyl)piperazinyl-1-)piperidyl, 4-(N-(2-N,N-dimethylaminoethyl)piperazinyl-1-)piperidyl, 4-(N-(2-N,N-diethylethyl)piperazinyl-1-)piperidyl, 4-(N-(3-N,N-dimethylpropyl)piperazinyl-1-)piperidyl, 4-(N-(3-N,N-diethylpropyl)piperazinyl-1-)piperidyl, 4-(tetrahydropyrrolyl-1-)piperidyl, 4-(3-N,N-dimethylaminotetrahydropyrrolyl-1-)piperidyl, 4-(3-N,N-diethylaminotetrahydropyrrolyl-1-)piperidyl,
   (8) piperidin-4-yl, N-methyl-4-piperidyl, N-ethyl-4-piperidyl, N-isopropyl-4-piperidyl, N-(2-hydroxyethyl)-4-piperidyl, N-(2-N,N-dimethylaminoethyl)-4-piperidyl, N-(2-N,N-diethylaminoethyl)-4-piperidyl, N-(2-cyanoethyl)-4-piperidyl, N-(3-hydroxypropyl)-4-piperidyl, N-(3-N,N-dimethylaminopropyl)-4-piperidyl, N-(3-N,N-diethylaminopropyl)-4-piperidyl, N-(3-cyanopropyl)-4-piperidyl, N-cyanomethylene-4-piperidyl,
   (9) amino, N,N-dimethylamino, N,N-diethylamino, N,N-di-n-propylamino, N,N-diisopropylamino, 2-N,N-dimethylaminoethylamino, 3-N,N-dimethylaminopropylamino, 3-N,N-diethylaminopropylamino, 3-N,N-diisopropylaminopropylamino, N-methylpiperidyl-4-amino, N-ethylpiperidyl-4-amino, N-n-propylpiperidyl-4-amino, N-isopropylpiperidyl-4-amino, acetamido, propionamido, 2-(N,N-dimethylamino)acetamido, 2-hydroxyacetamido, 2-methoxyacetamido, methylsulfamido, ethylsulfamido, n-propylsulfamido, isopropylsulfamido, cyclopropylsulfamido, N-methyl-N-(N-methyl-3-tetrahydropyrrolyl)amino, N-(N-methyl-3-tetrahydropyrrolyl)amino, N-methyl-N-(2-N,N-dimethylamino)ethylamino,
   (10) 4-methyl-1-piperazinylmethylene, 4-ethyl-1-piperazinylmethylene, 4-propyl-1-piperazinylmethylene, 4-isopropyl-1-piperazinylmethylene, 4-(2-hydroxyethyl)-1-piperazinylmethylene, 4-(2-N,N-dimethylaminoethyl)-1-piperazinylmethylene, piperidyl-1-methylene, 4-(N,N-dimethylamino)-1-piperidylmethylene, 4-hydroxypiperidyl-1-methylene, tetrahydropyrrolyl-1-methylene, 3-(N,N-dimethylamino)-1-tetrahydropyrrolylmethylene, benzyl, cyclopropylmethylene, cyclobutylmethylene, cyclopentylmethylene, cyclohexylmethylene,
   (11) aminoformyl, methylaminoformyl, N,N-dimethylaminoformyl, ethylaminoformyl, aminoacetyl, methylaminoacetyl, 2-(N,N-dimethylamino)acetyl, ethylaminoacetyl, tetrahydropyrrolyl-1-formyl, 3-(N,N-dimethylamino)tetrahydropyrrolyl-1-formyl, 3-(N,N-diethylamino)tetrahydropyrrolyl-1-formyl, morpholinyl-4-formyl, thiomorpholinyl-4-formyl, 2,6-dimethylmorpholinyl-4-formyl, piperidyl-1-formyl, 4-hydroxypiperidyl-1-formyl, 4-(N,N-dimethylamino)piperidyl-1-formyl, 4-(N,N-diethylamino)piperidyl-1-formyl, 4-(N-methyl-1-piperazinyl)piperidyl-1-formyl, 4-(N-ethyl-1-piperazinyl)piperidyl-1-formyl, N-methylpiperazinyl-1-formyl, N-ethylpiperazinyl-1-formyl, N-isopropylpiperazinyl-1-formyl, N-(2-hydroxyethyl)piperazinyl-1-formyl, N-(2-N,N-dimethylaminoethyl)piperazinyl-1-formyl, N-(2-N,N-diethylaminoethyl)piperazinyl-1-formyl, 4-(N-methyl-4-piperidyl)piperazinyl-1-formyl, 4-(N-ethyl-4-piperidyl)piperazinyl-1-formyl,
   (12) hydroxysulfonyl, aminosulfonyl, methylaminosulfonyl, ethylaminosulfonyl, propylaminosulfonyl, isopropylaminosulfonyl, cyclopropylaminosulfonyl, cyclobutylaminosulfonyl, cyclopentylaminosulfonyl, cyclohexylaminosulfonyl, tetrahydropyrrolyl-1-sulfonyl, 3-(N,N-dimethylamino)tetrahydropyrrolyl-1-sulfonyl, 3-(N,N-diethylamino)tetrahydropyrrolyl-1-sulfonyl, morpholinyl-4-sulfonyl, thiomorpholinyl-4-sulfonyl, 2,6-dimethylmorpholinyl-4-sulfonyl, piperidyl-1-sulfonyl, 4-hydroxypiperidyl-1-sulfonyl, 4-(N,N-dimethylamino)piperidyl-1-sulfonyl, 4-(N,N-diethylamino)piperidyl-1-sulfonyl, N-methylpiperazinyl-1-sulfonyl, N-ethylpiperazinyl-1-sulfonyl, N-isopropylpiperazinyl-1-sulfonyl, N-(2-hydroxyethyl)piperazinyl-1-sulfonyl, N-(2-N,N-dimethylaminoethyl)piperazinyl-1-sulfonyl, N-(2-N,N-diethylaminoethyl)piperazinyl-1-sulfonyl,
   (13) aminoformamido, methylaminoformamido, ethylaminoformamido, propylaminoformamido, isopropylaminoformamido;
5)

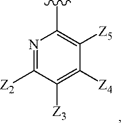

wherein Z₂, Z₃, Z₄, Z₅ are the same as defined in 4), and Z₂, Z₃, Z₄, Z₅ are not hydrogen at the same time;
6)

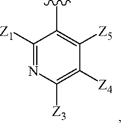

wherein Z₁, Z₃, Z₄, Z₅ are the same as defined in 4), and Z₁, Z₃, Z₄, Z₅ are not hydrogen at the same time;
7)

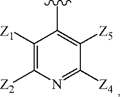

wherein Z₁, Z₂, Z₄, Z₅ are the same as defined in 4), and Z₁, Z₂, Z₄, Z₅ may be hydrogen at the same time;
8)

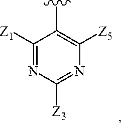

wherein Z₁, Z₃, Z₅ are the same as defined in 4), and Z₁, Z₃, Z₅ are not hydrogen at the same time;
9)

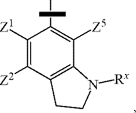

wherein Z₁, Z₂, Z₅ are the same as defined in 4), and Z₁, Z₂, Z₅ may be hydrogen at the same time, R^{X} is selected from the same substituents as described above for Z¹;
10)

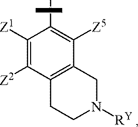

wherein Z₁, Z₂, Z₅ are the same as defined in 4), and Z₁, Z₂, Z₅ may be hydrogen at the same time, R^{y} is selected from the same substituents as described above for Z¹;
11)

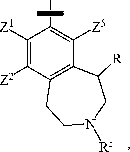

wherein Z₁, Z₂, Z₅ are the same as defined in 4), and Z₁, Z₂, Z₅ may be hydrogen at the same time, R^{z}, R each are independently selected from the same substituents as described above for Z¹.

In some embodiments, R¹ is selected from:
1) H, C₁-C₆ alkyl, C₃-C₈ cycloalkyl;
2) 2-N,N-dimethylaminoethyl, 3-N,N-dimethylaminopropyl, 2-N,N-diethylaminoethyl, 3-N,N-diethylaminopropyl, 2-hydroxyethyl, 3-hydroxypropyl, N-methyl-4-piperidyl, N-ethyl-4-piperidyl, N-propyl-4-piperidyl, N-isopropyl-4-piperidyl, 4-aminocyclohexyl, 4-N,N-dimethylaminocyclohexyl, 4-N,N-diethylaminocyclohexyl, 4-N,N-dipropylaminocyclohexyl, 4-N,N-diisopropylaminocyclohexyl;
3) 1-methyl-3-pyrazolyl, 1-ethyl-3-pyrazolyl, 1-isopropyl-3-pyrazolyl, 1-cyclopropyl-3-pyrazolyl, 1-methyl-4-pyrazolyl, 1-ethyl-4-pyrazolyl, 1-isopropyl-4-pyrazolyl, 1-cyclopropyl-4-pyrazolyl, 1,3-dimethyl-5-pyrazolyl, 1-methyl-4-imidazolyl;
4)

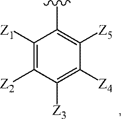

wherein Z₁, Z₂, Z₃, Z₄, Z₅ each are independently selected from the following groups, and Z₁, Z₂, Z₃, Z₄, Z₅ are not hydrogen at the same time:
   (1) H, F, Cl, Br, I, nitro, cyano,
   (2) C₁-C₆ alkyl, C₁-C₆ alkoxy, C₁-C₆ fluorine-containing alkyl, C₁-C₆ fluorine-containing alkoxy, C₂-C₆ alkenyl,
   (3) piperazinyl, N-methylpiperazinyl, N-ethylpiperazinyl, N-n-propylpiperazinyl, N-isopropylpiperazinyl,
   (4) morpholinyl, 2,6-dimethylmorpholinyl, thiomorpholinyl,
   (5) 3-(N,N-dimethylamino)tetrahydropyrrolyl, 3-(N,N-diethylamino)tetrahydropyrrolyl,
   (6) 4-N,N-dimethylaminopiperidyl, 4-N,N-diethylaminopiperidyl, 4-N,N-diisopropylaminopiperidyl, 4-hydroxypiperidyl, 4-(N-methylpiperazinyl-1-)piperidyl, 4-(N-ethylpiperazinyl-1-)piperidyl, 4-(N-isopropylpiperazinyl-1-)piperidyl, 4-(tetrahydropyrrolyl-1-)piperidyl,
   (7) piperidin-4-yl, N-methyl-4-piperidyl, N-ethyl-4-piperidyl, N-isopropyl-4-piperidyl,
   (8) aminoformamido, methylaminoformamido, ethylaminoformamido, propylaminoformamido, isopropylaminoformamido, 2-(N,N-dimethylamino)acetamido, cyclopropylsulfamido, 2-N,N-dimethylaminoethylamino, 3-N,N-dimethylaminopropylamino, 3-N,N-diethylaminopropylamino, 3-N,N-diisopropylaminopropylamino, N-methyl-N-(2-N,N-dimethylamino)ethylamino,
   (9) 4-methyl-1-piperazinylmethylene, 4-ethyl-1-piperazinylmethylene, 4-propyl-1-piperazinylmethylene, 4-isopropyl-1-piperazinylmethylene, benzyl, cyclopropylmethylene, cyclobutylmethylene, cyclopentylmethylene, cyclohexylmethylene,
   (10) 4-(N-methyl-1-piperazinyl)piperidyl-1-formyl, 4-(N-ethyl-1-piperazinyl)piperidyl-1-formyl, N-methylpiperazinyl-1-formyl, N-ethylpiperazinyl-1-formyl, N-isopropylpiperazinyl-1-formyl,
   (11) cyclopropylaminosulfonyl, cyclobutylaminosulfonyl, cyclopentylaminosulfonyl, cyclohexylaminosulfonyl, N-methylpiperazinyl-1-sulfonyl, N-ethylpiperazinyl-1-sulfonyl, N-isopropylpiperazinyl-1-sulfonyl;
5)

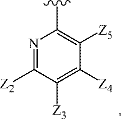

wherein Z₂, Z₃, Z₄, Z₅ each are independently selected from H, C₁-C₆ alkyl, C₁—C₆ alkoxy, piperazinyl, N-methylpiperazinyl, N-ethylpiperazinyl, N-n-propylpiperazinyl, or N-isopropylpiperazinyl, and Z₂, Z₃, Z₄, Z₅ are not hydrogen at the same time;
6)

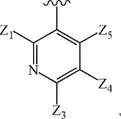

wherein Z₁, Z₃, Z₄, Z₅ each are independently selected from H, C₁—C₆ alkyl, C₁—C₆ alkoxy, N-(N-methyl-3-tetrahydropyrrolyl)amino, N-methyl-N-(N-methyl-3-tetrahydropyrrolyl)amino, N-methylpiperazinyl, N-ethylpiperazinyl, N-n-propylpiperazinyl, N-isopropylpiperazinyl, 4-(N-methylpiperazinyl-1-)piperidyl, 4-(N-ethylpiperazinyl-1-)piperidyl, or 4-(N-isopropylpiperazinyl-1-)piperidyl, and Z₁, Z₃, Z₄, Z₅ are not hydrogen at the same time;
7)

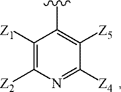

wherein Z₁, Z₂, Z₄, Z₅ each are independently selected from H, or C₁-C₆ alkyl, and Z₁, Z₂, Z₄, Z₅ may be hydrogen at the same time;
8)

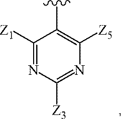

wherein Z₁, Z₃, Z₅ each are independently selected from H, C₁-C₆ alkyl, C₁-C₆ alkoxy, N-methylpiperazinyl, N-ethylpiperazinyl, N-n-propylpiperazinyl, or N-isopropylpiperazinyl, and Z₁, Z₃, Z₅ are not hydrogen at the same time;
9)

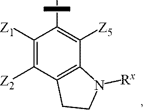

wherein Z₁, Z₂, Z₅ each are independently selected from H, C₁-C₆ alkyl, or C₁-C₆ alkoxy, and Z₁, Z₂, Z₅ may be hydrogen at the same time, R^{x} is selected from aminoformyl, methylaminoformyl, N,N-dimethylaminoformyl, ethylaminoformyl, aminoacetyl, methylaminoacetyl, 2-(N,N-dimethylamino)acetyl, or ethylaminoacetyl;
10)

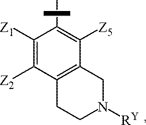

wherein Z₁, Z₂, Z₅ each are independently selected from H, F, Cl, Br, I, C₁-C₆ alkyl, C₁-C₆ alkoxy, C₁-C₆ fluorine-containing alkyl, or C₁-C₆ fluorine-containing alkoxy, and Z₁, Z₂, Z₅ may be hydrogen at the same time, R^{y} is C₁-C₆ alkyl;
11)

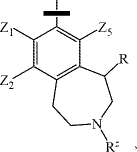

wherein Z₁, Z₂, Z₅ each are independently selected from H, or C₁-C₆ alkyl, and Z₁, Z₂, Z₅ may be hydrogen at the same time, R^{z}, R each are independently selected from C₁-C₆ alkyl.

In some embodiments, R¹ is selected from:
1) cyclopropyl, 2-N,N-dimethylaminoethyl, 2-hydroxyethyl, N-methyl-4-piperidyl, 4-aminocyclohexyl, 1-methyl-3-pyrazolyl, 1-methyl-4-pyrazolyl, 1,3-dimethyl-5-pyrazolyl, 1-methyl-4-imidazolyl;
2)

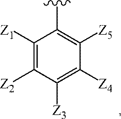

wherein Z₁, Z₂, Z₃, Z₄, Z₅ each are independently selected from the following groups, and Z₁, Z₂, Z₃, Z₄, Z₅ are not hydrogen at the same time:
   H, methyl, ethyl, methoxy, ethoxy, isopropoxy, trifluoromethyl, trifluoromethoxy, vinyl, N-methylpiperazinyl, morpholinyl, 3-(N,N-dimethylamino)tetrahydropyrrolyl, 4-N,N-dimethylaminopiperidyl, 4-hydroxypiperidyl, 4-(N-methylpiperazinyl-1-)piperidyl, 4-(tetrahydropyrrolyl-1-)piperidyl, N-methyl-4-piperidyl, 2-(N,N-dimethylamino)acetamido, cyclopropylsulfamido, N-methyl-N-(2-N,N-dimethylamino)ethylamino, 4-methyl-1-piperazinylmethylene, benzyl, cyclopropylmethylene, 4-(N-methyl-1-piperazinyl)piperidyl-1-formyl, N-methylpiperazinyl-1-formyl, cyclopropylaminosulfonyl, or N-methylpiperazinyl-1-sulfonyl;
3)

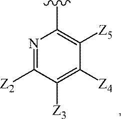

wherein Z₂, Z₃, Z₄, Z₅ each are independently selected from H, methoxy, or N-methylpiperazinyl, and Z₂, Z₃, Z₄, Z₅ are not hydrogen at the same time;
4)

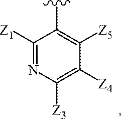

wherein Z₁, Z₃, Z₄, Z₅ each are independently selected from H, methyl, ethyl, methoxy, N-(N-methyl-3-tetrahydropyrrolyl)amino, N-methyl-N-(N-methyl-3-tetrahydropyrrolyl)amino, N-methylpiperazinyl, or 4-(N-methylpiperazinyl-1-)piperidyl, and Z₁, Z₃, Z₄, Z₅ are not hydrogen at the same time;
5)

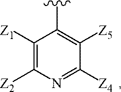

wherein Z₁, Z₂, Z₄, Z₅ are H;
6)

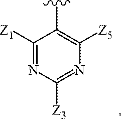

wherein Z₁, Z₃, Z₅ each are independently selected from H, methoxy, or N-methylpiperazinyl, and Z₁, Z₃, Z₅ are not hydrogen at the same time;
7)

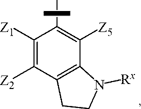

wherein Z₁, Z₂ Z₅ each are independently selected from H, or methoxy, R^{x} is 2-(N,N-dimethylamino)acetyl;
8)

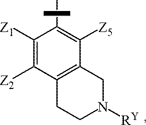

wherein Z₁, Z₂, Z₅ each are independently selected from H, F, Cl, methyl, methoxy, trifluoromethyl, or trifluoromethoxy, R^{y} is methyl;
9)

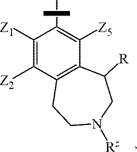

wherein Z₁, Z₂, Z₅ are H, R^{z}, R are methyl.

In some embodiments, R², R³ each are independently selected from methyl, trifluoromethyl, ethyl, isopropyl, or cyclopropyl.

In some embodiments, R⁴ is H, R⁵ is selected from F, Cl, Br, trifluoromethyl, cyano, nitro, methyl, methoxy or cyclopropyl, or, R⁴, R⁵ and carbon atoms linked thereto together form

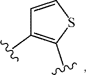

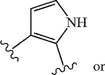

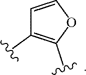

In the fifth aspect of the present invention, the present invention provides a compound having the general formula (I):

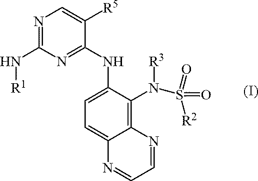

or a stereoisomer, a prodrug, a pharmaceutically acceptable salt or a pharmaceutically acceptable solvate thereof, wherein:
R¹ is selected from:
   1) H, C₁-C₆ alkyl, C₁-C₆ fluorine-containing alkyl, C₃-C₈ cycloalkyl, C₁-C₆ heteroatom-containing alkyl, C₃-C₈ heteroatom-containing cycloalkyl;
   2) 2-N,N-dimethylaminoethyl, 3-N,N-dimethylaminopropyl, 2-N,N-diethylaminoethyl, 3-N,N-diethylaminopropyl, 2-(N-methylpiperazinyl)ethyl, 2-(N-acetylpiperazinyl)ethyl, 2-morpholinylethyl, 2-thiomorpholinylethyl, 2-piperidylethyl, 2-hydroxyethyl, 3-hydroxypropyl, 2-methoxyethyl, N-methyl-4-piperidyl, N-ethyl-4-piperidyl, N-propyl-4-piperidyl, N-isopropyl-4-piperidyl, N-hydroxyethyl-4-piperidyl, N-cyanomethyl-4-piperidyl, N-(2-cyanoethyl)-4-piperidyl, N-acetyl-4-piperidyl, N-propionyl-4-piperidyl, N-isopropionyl-4-piperidyl, N-cyclopropylformyl-4-piperidyl, N-methylsulfonyl-4-piperidyl, N-ethylsulfonyl-4-piperidyl, N-propylsulfonyl-4-piperidyl, N-isopropylsulfonyl-4-piperidyl, N-cyclopropylsulfonyl-4-piperidyl, 3-N,N-dimethylaminocyclopentyl, 3-N,N-diethylaminocyclopentyl, 3-N,N-dipropylaminocyclopentyl, 3-N,N-isopropylaminocyclopentyl, 4-aminocyclohexyl, 4-N,N-dimethylaminocyclohexyl, 4-N,N-diethylaminocyclohexyl, 4-N,N-dipropylaminocyclohexyl, 4-N,N-diisopropylaminocyclohexyl, 4-acetylaminocyclohexyl, 4-propionylaminocyclohexyl, 4-isopropylformylaminocyclohexyl, 4-methylsulfonylaminocyclohexyl, 4-ethylsulfonylaminocyclohexyl, 4-propylsulfonylaminocyclohexyl, 4-isopropylsulfonylaminocyclohexyl, 4-cyclopropylsulfonylaminocyclohexyl, 4-hydroxycyclohexyl, 4-methoxycyclohexyl, 4-ethoxycyclohexyl, 4-isopropoxycyclohexyl;
   3) 1-methyl-3-pyrrolyl, 1-ethyl-3-pyrrolyl, 1-isopropyl-3-pyrrolyl, 1-cyclopropyl-3-pyrrolyl, 1-methyl-3-pyrazolyl, 1-ethyl-3-pyrazolyl, 1-isopropyl-3-pyrazolyl, 1-cyclopropyl-3-pyrazolyl, 1-hydroxyethyl-3-pyrazolyl, 1-methyl-4-pyrazolyl, 1-ethyl-4-pyrazolyl, 1-isopropyl-4-pyrazolyl, 1-cyclopropyl-4-pyrazolyl, 1-hydroxyethyl-4-pyrazolyl, 1-(N-methyl-4-piperidyl)-4-pyrazolyl, 1-(N-t-butoxycarbonyl-4-piperidyl)-4-pyrazolyl, 1,3-dimethyl-5-pyrazolyl, 1-methyl-4-imidazolyl, 3-methyl-5-isoxazolyl;
   4) 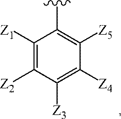
   wherein Z₁, Z₂, Z₃, Z₄, Z₅ each are independently selected from the following groups, and Z₁, Z₂, Z₃, Z₄, Z₅ are not hydrogen at the same time:
      (1) H, F, Cl, Br, I, nitro, cyano,
      (2) C₁-C₆ alkyl, C₁-C₆ alkoxy, C₁-C₆ oxygen-containing alkyl, C₁-C₆ fluorine-containing alkyl, C₁-C₆ fluorine-containing alkoxy, C₃-C₆ cycloalkyl, C₂-C₆ alkenyl,
      (3) 2-N,N-dimethylaminoethoxy, 3-N,N-dimethylaminopropoxy, 2-(N-methylpiperazinyl)ethoxy, 2-(N-acetylpiperazinyl)ethoxy, 2-morpholinylethoxy, 2-thiomorpholinylethoxy, 2-piperidylethoxy, 3-N,N-dimethylaminopropoxy, 3-N,N-diethylaminopropoxy, 3-N,N-diisopropylaminopropoxy, 3-(N-methylpiperazinyl)propoxy, 3-(N-acetylpiperazinyl)propoxy, 3-morpholinylpropoxy, 3-thiomorpholinylpropoxy, 3-piperidylpropoxy, 2-pyridylmethoxy, 3-pyridylmethoxy, 4-pyridylmethoxy, phenylmethoxy, monohalo-substituted phenylmethoxy, gem-dihalo-substituted phenylmethoxy, hetero-dihalo-substituted phenylmethoxy,
      (4) piperazinyl, N-methylpiperazinyl, N-ethylpiperazinyl, N-n-propylpiperazinyl, N-isopropylpiperazinyl, N-cyclopropylpiperazinyl, N-(2-hydroxyethyl)piperazinyl, N-(2-N,N-dimethylaminoethyl)piperazinyl, N-(2-N,N-diethylaminoethyl)piperazinyl, N-(2-cyanoethyl)piperazinyl, N-cyanomethylpiperazinyl, N-(3-hydroxypropyl)piperazinyl, N-(3-N,N-dimethylaminopropyl)piperazinyl, N-(3-N,N-diethylaminopropyl)piperazinyl, N-(N-methyl-4-piperidyl)piperazinyl, N-(N-ethyl-4-piperidyl)piperazinyl, N-t-butoxycarbonylpiperazinyl, N-acetylpiperazinyl, N-propionylpiperazinyl, N-isobutyrylpiperazinyl, N-cyclopropylformylpiperazinyl, N-methylsulfonylpiperazinyl, N-ethylsulfonylpiperazinyl, N-n-propylsulfonylpiperazinyl, N-isopropylsulfonylpiperazinyl, N-cyclopropylsulfonylpiperazinyl, 2-oxo-piperazin-4-yl,
      (5) morpholinyl, 2,6-dimethylmorpholinyl, thiomorpholinyl,
      (6) tetrahydropyrrolyl, 3-(N,N-dimethylamino)tetrahydropyrrolyl, 3-(N,N-diethylamino)tetrahydropyrrolyl,
      (7) imidazolyl, 4-methyl-1-imidazolyl,
      (8) piperidyl, 4-N,N-dimethylaminopiperidyl, 4-N,N-diethylaminopiperidyl, 4-N,N-diisopropylaminopiperidyl, 4-hydroxypiperidyl, 4-methoxypiperidyl, 4-ethoxypiperidyl, 4-(N-methylpiperazinyl-1-)piperidyl, 4-(N-ethylpiperazinyl-1-)piperidyl, 4-(N-isopropylpiperazinyl-1-)piperidyl, 4-(N-acetylpiperazinyl-1-)piperidyl, 4-(N-t-butoxycarbonylpiperazinyl-1-)piperidyl, 4-(N-methylsulfonylpiperazinyl-1-)piperidyl, 4-(N-(2-hydroxyethyl)piperazinyl-1-)piperidyl, 4-(N-(2-cyanoethyl)piperazinyl-1-)piperidyl, 4-(N-(3-hydroxypropyl)piperazinyl-1-)piperidyl, 4-(N-(2-N,N-dimethylaminoethyl)piperazinyl-1-)piperidyl, 4-(N-(2-N,N-diethylethyl)piperazinyl-1-)piperidyl, 4-(N-(3-N,N-dimethylpropyl)piperazinyl-1-)piperidyl, 4-(N-(3-N,N-diethylpropyl)piperazinyl-1-)piperidyl, 4-(tetrahydropyrrolyl-1-)piperidyl, 4-(3-N,N-dimethylaminotetrahydropyrrolyl-1-)piperidyl, 4-(3-N,N-diethylaminotetrahydropyrrolyl-1-)piperidyl,
      (9) piperidin-4-yl, N-methyl-4-piperidyl, N-ethyl-4-piperidyl, N-isopropyl-4-piperidyl, N-acetyl-4-piperidyl, N-t-butoxycarbonyl-4-piperidyl, N-methylsulfonyl-4-piperidyl, N-(2-hydroxyethyl)-4-piperidyl, N-(2-N,N-dimethylaminoethyl)-4-piperidyl, N-(2-N,N-diethylaminoethyl)-4-piperidyl, N-(2-cyanoethyl)-4-piperidyl, N-(3-hydroxypropyl)-4-piperidyl, N-(3-N,N-dimethylaminopropyl)-4-piperidyl, N-(3-N,N-diethylaminopropyl)-4-piperidyl, N-(3-cyanopropyl)-4-piperidyl, N-cyanomethylene-4-piperidyl,
      (10) amino, N,N-dimethylamino, N,N-diethylamino, N,N-di-n-propylamino, N,N-diisopropylamino, 2-N,N-dimethylaminoethylamino, 2-morpholinylethylamino, 2-(N-methylpiperazinyl)ethylamino, 3-N,N-dimethylaminopropylamino, 3-N,N-diethylaminopropylamino, 3-N,N-diisopropylaminopropylamino, 3-morpholinylpropylamino, 3-(N-methylpiperazinyl)propylamino, N-methylpiperidyl-4-amino, N-ethylpiperidyl-4-amino, N-n-propylpiperidyl-4-amino, N-isopropylpiperidyl-4-amino, acetamido, propionamido, 2-(N,N-dimethylamino)acetamido, 2-hydroxyacetamido, 2-methoxyacetamido, methylsulfamido, ethylsulfamido, n-propylsulfamido, isopropylsulfamido, cyclopropylsulfamido, N-methyl-N-(N-methyl-3-tetrahydropyrrolyl)amino, N-(N-methyl-3-tetrahydropyrrolyl)amino, N-methyl-N-(2-N,N-dimethylamino)ethylamino,
      (11) aminomethylene, N,N-dimethylaminomethylene, N,N-diethylaminomethylene, 4-methyl-1-piperazinylmethylene, 4-ethyl-1-piperazinylmethylene, 4-propyl-1-piperazinylmethylene, 4-isopropyl-1-piperazinylmethylene, 4-(2-hydroxyethyl)-1-piperazinylmethylene, 4-(2-N,N-dimethylaminoethyl)-1-piperazinylmethylene, piperidyl-1-methylene, 4-(N,N-dimethylamino)-1-piperidylmethylene, 4-hydroxypiperidyl-1-methylene, tetrahydropyrrolyl-1-methylene, 3-(N,N-dimethylamino)-1-tetrahydropyrrolylmethylene, benzyl, cyclopropylmethylene, cyclobutylmethylene, cyclopentylmethylene, cyclohexylmethylene,
      (12) aminoformyl, methylaminoformyl, N,N-dimethylaminoformyl, ethylaminoformyl, cyclopropylaminoformyl, cyclobutylaminoformyl, cyclopentylaminoformyl, cyclohexylaminoformyl, aminoacetyl, methylaminoacetyl, 2-(N,N-dimethylamino)acetyl, ethylam inoacetyl, cyclopropylam inoacetyl, cyclobutylam inoacetyl, cyclopentylaminoacetyl, cyclohexylaminoacetyl, tetrahydropyrrolyl-1-formyl, 3-(N, N-dimethylamino)tetrahydropyrrolyl-1 -formyl, 3-(N, N-diethylam ino)tetrahydropyrrolyl-1-formyl, morpholinyl-4-formyl, thiomorpholinyl-4-formyl, 2,6-dimethylmorpholinyl-4-formyl, piperidyl-1-formyl, 4-hydroxypiperidyl-1-formyl, 4-(N,N-dimethylamino)piperidyl-1-formyl, 4-(N,N-diethylamino)piperidyl-1-formyl, 4-(N-methyl-1-piperazinyl)piperidyl-1-formyl, 4-(N-ethyl-1-piperazinyl)piperidyl-1-formyl, N-methylpiperazinyl-1-formyl, N-ethylpiperazinyl-1-formyl, N-isopropylpiperazinyl-1-formyl, N-(2-hydroxyethyl)piperazinyl-1-formyl, N-(2-N, N-dimethylaminoethyl)piperazinyl-1-formyl, N-(2-N, N-diethylaminoethyl)piperazinyl-1-formyl, 4-(N-methyl-4-piperidyl)piperazinyl-1-formyl, 4-(N-ethyl-4-piperidyl)piperazinyl-1-formyl,
      (13) hydroxyformyl, methoxyformyl, ethoxyformyl, propoxyformyl, isopropoxyformyl, n-butoxyformyl, iso-butoxyformyl, t-butoxyformyl,
      (14) hydroxysulfonyl, aminosulfonyl, methylaminosulfonyl, ethylaminosulfonyl, propylaminosulfonyl, isopropylaminosulfonyl, cyclopropylaminosulfonyl, cyclobutylaminosulfonyl, cyclopentylaminosulfonyl, cyclohexylaminosulfonyl, tetrahydropyrrolyl-1-sulfonyl, 3-(N,N-dimethylamino)tetrahydropyrrolyl-1-sulfonyl, 3-(N, N-diethylam ino)tetrahydropyrrolyl-1-sulfonyl, morpholinyl-4-sulfonyl, thiomorpholinyl-4-sulfonyl, 2,6-dimethylmorpholinyl-4-sulfonyl, piperidyl-1-sulfonyl, 4-hydroxypiperidyl-1-sulfonyl, 4-(N,N-dimethylamino)piperidyl-1-sulfonyl, 4-(N,N-diethylamino)piperidyl-1-sulfonyl, 4-(N-methyl-1-piperazinyl)piperidyl-1-sulfonyl, 4-(N-ethyl-1-piperazinyl)piperidyl-1-sulfonyl, N-methylpiperazinyl-1-sulfonyl, N-ethylpiperazinyl-1-sulfonyl, N-isopropylpiperazinyl-1-sulfonyl, N-(2-hydroxyethyl)piperazinyl-1-sulfonyl, N-(2-N,N-dimethylaminoethyl)piperazinyl-1-sulfonyl, N-(2-N,N-diethylaminoethyl)piperazinyl-1-sulfonyl, 4-(N-methyl-4-piperidyl)piperazinyl-1-sulfonyl, 4-(N-ethyl-4-piperidyl)piperazinyl-1-sulfonyl,
      (15) aminoformamido, methylaminoformamido, ethylaminoformamido, propylaminoformamido, isopropylaminoformamido, cyclopropylaminoformamido, cyclobutylaminoformamido, cyclopentylaminoformamido, piperidyl-1-formamido, 4-hydroxypiperidyl-1-formamido, 4-N,N-dimethylpiperidyl-1-formamido, 4-N, N-diethylpiperidyl-1 -formamido, tetrahydropyrrolyl-1 -formam ido, 3-N, N-dimethyltetrahydropyrrolyl-1 -formam ido, 3-N,N-diethyltetrahydropyrrolyl-1-formamido, N-methylpiperazinyl-1-formamido, N-ethylpiperazinyl-1-formamido, N-acetylpiperazinyl-1-formamido, N-t-butoxycarbonylpiperazinyl-1-formamido, N-(2-hydroxyethyl)piperazinyl-1-formamido, N-(2-cyanoethyl)piperazinyl-1 -formam ido, N-(2-N, N-dimethylam inoethyl)piperazinyl-1-formam ido, N-(2-N, N-diethylam inoethyl)piperazinyl-1-formam ido, N-(3-hydroxypropyl)piperazinyl-1 -formam ido, N-(3-N,N-dimethylpropyl)piperazinyl-1 -formamido, N-(3-N,N-diethylpropyl)piperazinyl-1-formam ido, morpholinyl-1-formam ido, 3,5-dimethylmorpholinyl-1-formamido, 4-(tetrahydropyrrolyl)piperidyl-1-formamido, 4-(N-methyl-1-piperazinyl)piperidyl-1-formam ido, 4-(N-ethyl-1 -piperazi nyl)piperidyl-1 -form am ido, 4-(N-acetyl-1-piperazinyl)piperidyl-1-formamido, N-(N-methyl-4-piperidyl)piperazinyl-1-formam ido,
      (16) Z₃ and Z₄ may form an oxygen-containing substituted or unsubstituted 5- to 7-membered ring; the substituent may be selected from the same substituents as described above for Z₁,
      (17) Z₃ and Z₄ may form a nitrogen-containing substituted or unsubstituted 5- to 7-membered ring; the substituent may be selected from the same substituents as described above for Z₁,
   5) 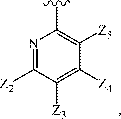
   wherein Z₂, Z₃, Z₄, Z₅ are the same as defined in 4), and Z₂, Z₃, Z₄, Z₅ are not hydrogen at the same time;
   6) 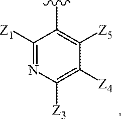
   wherein Z₁, Z₃, Z₄, Z₅ are the same as defined in 4), and Z₁, Z₃, Z₄, Z₅ are not hydrogen at the same time;
   7) 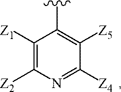
   wherein Z₁, Z₂, Z₄, Z₅ are the same as defined in 4), and Z₁, Z₂, Z₄, Z₅ may be hydrogen at the same time;
   8) 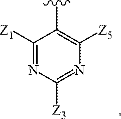
   wherein Z₁, Z₃, Z₅ are the same as defined in 4), and Z₁, Z₃, Z₅ are not hydrogen at the same time;
R², R³ each are independently selected from H, C₁-C₆ alkyl, C₁-C₆ fluorine-containing alkyl, C₃-C₆ cycloalkyl, or C₃-C₆ fluorine-containing cycloalkyl;
R⁵ is selected from F, Cl, Br, C₁-C₆ fluorine-containing alkyl, cyano, nitro, C₁-C₆ alkyl, C₁-C₆ alkoxy, C₃-C₆ cycloalkyl.

In some embodiments, R¹ is selected from:
1) H, C₁-C₆ alkyl, C₁-C₆ fluorine-containing alkyl, C₃-C₈ cycloalkyl, C₁-C₆ heteroatom-containing alkyl, C₃-C₈ heteroatom-containing cycloalkyl;
2) 2-N,N-dimethylaminoethyl, 3-N,N-dimethylaminopropyl, 2-N,N-diethylaminoethyl, 3-N,N-diethylaminopropyl, 2-(N-methylpiperazinyl)ethyl, 2-(N-acetylpiperazinyl)ethyl, 2-morpholinylethyl, 2-thiomorpholinylethyl, 2-piperidylethyl, 2-hydroxyethyl, 3-hydroxypropyl, 2-methoxyethyl, N-methyl-4-piperidyl, N-ethyl-4-piperidyl, N-propyl-4-piperidyl, N-isopropyl-4-piperidyl, N-hydroxyethyl-4-piperidyl, N-cyanomethyl-4-piperidyl, N-(2-cyanoethyl)-4-piperidyl, 3-N,N-dimethylaminocyclopentyl, 3-N,N-diethylaminocyclopentyl, 3-N,N-dipropylaminocyclopentyl, 3-N,N-isopropylaminocyclopentyl, 4-aminocyclohexyl, 4-N,N-dimethylaminocyclohexyl, 4-N,N-diethylaminocyclohexyl, 4-N,N-dipropylaminocyclohexyl, 4-N,N-diisopropylaminocyclohexyl, 4-hydroxycyclohexyl, 4-methoxycyclohexyl, 4-ethoxycyclohexyl, 4-isopropoxycyclohexyl;
3) 1-methyl-3-pyrrolyl, 1-ethyl-3-pyrrolyl, 1-isopropyl-3-pyrrolyl, 1-cyclopropyl-3-pyrrolyl, 1-methyl-3-pyrazolyl, 1-ethyl-3-pyrazolyl, 1-isopropyl-3-pyrazolyl, 1-cyclopropyl-3-pyrazolyl, 1-hydroxyethyl-3-pyrazolyl, 1-methyl-4-pyrazolyl, 1-ethyl-4-pyrazolyl, 1 -isopropyl-4-pyrazolyl, 1-cyclopropyl-4-pyrazolyl, 1-hydroxyethyl-4-pyrazolyl, 1,3-dimethyl-5-pyrazolyl, 1-methyl-4-imidazolyl, 3-methyl-5-isoxazolyl;
4)

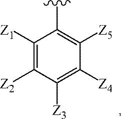

wherein Z₁, Z₂, Z₃, Z₄, Z₅ each are independently selected from the following groups, and Z₁, Z₂, Z₃, Z₄, Z₅ are not hydrogen at the same time:
   (1) H, F, Cl, Br, l, nitro, cyano,
   (2) C₁-C₆ alkyl, C₁-C₆ alkoxy, C₁-C₆ oxygen-containing alkyl, C₁-C₆ fluorine-containing alkyl, C₁-C₆ fluorine-containing alkoxy, C₃-C₆ cycloalkyl, C₂-C₆ alkenyl,
   (3) piperazinyl, N-methylpiperazinyl, N-ethylpiperazinyl, N-n-propylpiperazinyl, N-isopropylpiperazinyl, N-cyclopropylpiperazinyl, N-(2-hydroxyethyl)piperazinyl, N-(2-N,N-dimethylaminoethyl)piperazinyl, N-(2-N,N-diethylaminoethyl)piperazinyl, N-(2-cyanoethyl)piperazinyl, N-cyanomethylpiperazinyl, N-(3-hydroxypropyl)piperazinyl, N-(3-N,N-dimethylaminopropyl)piperazinyl, N-(3-N,N-diethylaminopropyl)piperazinyl,
   (4) morpholinyl, 2,6-dimethylmorpholinyl, thiomorpholinyl,
   (5) tetrahydropyrrolyl, 3-(N,N-dimethylamino)tetrahydropyrrolyl, 3-(N, N-diethylam ino)tetrahydropyrrolyl,
   (6) imidazolyl, 4-methyl-1-imidazolyl,
   (7) piperidyl, 4-N,N-dimethylaminopiperidyl, 4-N,N-diethylaminopiperidyl, 4-N,N-diisopropylaminopiperidyl, 4-hydroxypiperidyl, 4-methoxypiperidyl, 4-ethoxypiperidyl, 4-(N-methylpiperazinyl-1 -)piperidyl, 4-(N-ethylpiperazinyl-1 -)piperidyl, 4-(N-isopropylpiperazinyl-1 -)piperidyl, 4-(N-(2-hydroxyethyl)piperazinyl-1 -)piperidyl, 4-(N-(2-cyanoethyl)piperazinyl-1 -)piperidyl, 4-(N-(3-hydroxypropyl)piperazinyl-1 -)piperidyl, 4-(N-(2-N, N-dimethylam inoethyl)piperazinyl-1 -)piperidyl, 4-(N-(2-N,N-diethylethyl)piperazinyl-1 -)piperidyl, 4-(N-(3-N,N-dimethylpropyl)piperazinyl-1 -)piperidyl, 4-(N-(3-N,N-diethylpropyl)piperazinyl-1-)piperidyl, 4-(tetrahydropyrrolyl-1-)piperidyl, 4-(3-N, N-dimethylam inotetrahydropyrrolyl-1 -)piperidyl, 4-(3-N, N-diethylam inotetrahydropyrrolyl-1 -)piperidyl,
   (8) piperidin-4-yl, N-methyl-4-piperidyl, N-ethyl-4-piperidyl, N-isopropyl-4-piperidyl, N-(2-hydroxyethyl)-4-piperidyl, N-(2-N, N-dimethylam inoethyl)-4-piperidyl, N-(2-N, N-diethylam inoethyl)-4-piperidyl, N-(2-cyanoethyl)-4-piperidyl, N-(3-hydroxypropyl)-4-piperidyl, N-(3-N, N-dimethylam inopropyl)-4-piperidyl, N-(3-N, N-diethylam inopropyl)-4-piperidyl, N-(3-cyanopropyl)-4-piperidyl, N-cyanomethylene-4-piperidyl,
   (9) amino, N,N-dimethylamino, N,N-diethylamino, N,N-di-n-propylamino, N, N-diisopropylam ino, 2-N, N-dimethylam inoethylam ino, 3-N, N-dimethylam inopropylam ino, 3-N, N-diethylam inopropylam ino, 3-N, N-diisopropylam inopropylam ino, N-methylpiperidyl-4-am ino, N-ethylpiperidyl-4-amino, N-n-propylpiperidyl-4-amino, N-isopropylpiperidyl-4-amino, acetamido, propionamido, 2-(N,N-dimethylamino)acetamido, 2-hydroxyacetamido, 2-methoxyacetamido, methylsulfamido, ethylsulfamido, n-propylsulfamido, isopropylsulfamido, cyclopropylsulfamido, N-methyl-N-(N-methyl-3-tetrahydropyrrolyl)amino, N-(N-methyl-3-tetrahydropyrrolyl)amino, N-methyl-N-(2-N,N-dimethylamino)ethylamino,
   (10) 4-methyl-1-piperazinylmethylene, 4-ethyl-1-piperazinylmethylene, 4-propyl-1-piperazinylmethylene, 4-isopropyl-1-piperazinylmethylene, 4-(2-hydroxyethyl)-1-piperazinylmethylene, 4-(2-N,N-dimethylam inoethyl)-1-piperazinylmethylene, piperidyl-1-methylene, 4-(N,N-dimethylamino)-1-piperidylmethylene, 4-hydroxypiperidyl-1-methylene, tetrahydropyrrolyl-1-methylene, 3-(N,N-dimethylamino)-1-tetrahydropyrrolylmethylene, benzyl, cyclopropylmethylene, cyclobutylmethylene, cyclopentylmethylene, cyclohexylmethylene,
   (11) aminoformyl, methylaminoformyl, N,N-dimethylaminoformyl, ethylaminoformyl, aminoacetyl, methylaminoacetyl, 2-(N,N-dimethylamino)acetyl, ethylaminoacetyl, tetrahydropyrrolyl-1-formyl, 3-(N, N-dimethylam ino)tetrahydropyrrolyl-1 -formyl, 3-(N, N-diethylam ino)tetrahydropyrrolyl-1-formyl, morpholinyl-4-formyl, thiomorpholinyl-4-formyl, 2,6-dimethylmorpholinyl-4-formyl, piperidyl-1-formyl, 4-hydroxypiperidyl-1-formyl, 4-(N,N-dimethylamino)piperidyl-1-formyl, 4-(N,N-diethylamino)piperidyl-1-formyl, 4-(N-methyl-1-piperazinyl)piperidyl-1-formyl, 4-(N-ethyl-1-piperazinyl)piperidyl-1-formyl, N-methylpiperazinyl-1-formyl, N-ethylpiperazinyl-1-formyl, N-isopropylpiperazinyl-1-formyl, N-(2-hydroxyethyl)piperazinyl-1-formyl, N-(2-N, N-dimethylam inoethyl)piperazinyl-1-formyl, N-(2-N, N-diethylam inoethyl)piperazinyl-1-formyl, 4-(N-methyl-4-piperidyl)piperazinyl-1-formyl, 4-(N-ethyl-4-piperidyl)piperazinyl-1-formyl,
   (12) hydroxysulfonyl, aminosulfonyl, methylaminosulfonyl, ethylaminosulfonyl, propylaminosulfonyl, isopropylaminosulfonyl, cyclopropylaminosulfonyl, cyclobutylaminosulfonyl, cyclopentylaminosulfonyl, cyclohexylaminosulfonyl, tetrahydropyrrolyl-1-sulfonyl, 3-(N,N-dimethylamino)tetrahydropyrrolyl-1-sulfonyl, 3-(N, N-diethylam ino)tetrahydropyrrolyl-1-sulfonyl, morpholinyl-4-sulfonyl, thiomorpholinyl-4-sulfonyl, 2,6-dimethylmorpholinyl-4-sulfonyl, piperidyl-1-sulfonyl, 4-hydroxypiperidyl-1-sulfonyl, 4-(N,N-dimethylamino)piperidyl-1-sulfonyl, 4-(N,N-diethylamino) piperidyl-1-sulfonyl, N-methylpiperazinyl-1-sulfonyl, N-ethylpiperazinyl-1-sulfonyl, N-isopropylpiperazinyl-1-sulfonyl, N-(2-hydroxyethyl)piperazinyl-1-sulfonyl, N-(2-N,N-dimethylam inoethyl)piperazinyl-1-sulfonyl, N-(2-N,N-diethylam inoethyl)piperazinyl-1-sulfonyl,
   (13) aminoformamido, methylaminoformamido, ethylaminoformamido, propylaminoformamido, isopropylaminoformamido;
5)

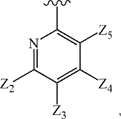

wherein Z₂, Z₃, Z₄, Z₅ are the same as defined in 4), and Z₂, Z₃, Z₄, Z₅ are not hydrogen at the same time;
6)

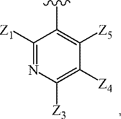

wherein Z₁, Z₃, Z₄, Z₅ are the same as defined in 4), and Z₁, Z₃, Z₄, Z₅ are not hydrogen at the same time;
7)

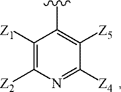

wherein Z₁, Z₂, Z₄, Z₅ are the same as defined in 4), and Z₁, Z₂, Z₄, Z₅ may be hydrogen at the same time;
8)

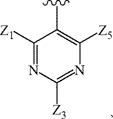

wherein Z₁, Z₃, Z₅ are the same as defined in 4), and Z₁, Z₃, Z₅ are not hydrogen at the same time;
9)

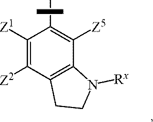

wherein Z₁, Z₂, Z₅ are the same as defined in 4), and Z₁, Z₂, Z₅ may be hydrogen at the same time, R^{X} is selected from the same substituents as described above for Z¹;
10)

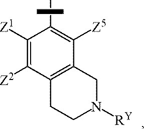

wherein Z₁, Z₂, Z₅ are the same as defined in 4), and Z₁, Z₂, Z₅ may be hydrogen at the same time, R^{y} is selected from the same substituents as described above for Z¹;
11)

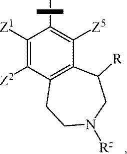

wherein Z₁, Z₂, Z₅ are the same as defined in 4), and Z₁, Z₂, Z₅ may be hydrogen at the same time, R^{z}, R each are independently selected from the same substituents as described above for Z¹.

In some embodiments, R¹ is selected from:
1) H, C₁-C₆ alkyl, C₃-C₈ cycloalkyl;
2) 2-N,N-dimethylaminoethyl, 3-N,N-dimethylaminopropyl, 2-N,N-diethylaminoethyl, 3-N,N-diethylaminopropyl, 2-hydroxyethyl, 3-hydroxypropyl, N-methyl-4-piperidyl, N-ethyl-4-piperidyl, N-propyl-4-piperidyl, N-isopropyl-4-piperidyl, 4-aminocyclohexyl, 4-N,N-dimethylaminocyclohexyl, 4-N,N-diethylaminocyclohexyl, 4-N,N-dipropylaminocyclohexyl, 4-N,N-diisopropylaminocyclohexyl;
3) 1-methyl-3-pyrazolyl, 1-ethyl-3-pyrazolyl, 1-isopropyl-3-pyrazolyl, 1-cyclopropyl-3-pyrazolyl, 1 -methyl-4-pyrazolyl, 1-ethyl-4-pyrazolyl, 1-isopropyl-4-pyrazolyl, 1-cyclopropyl-4-pyrazolyl, 1,3-dimethyl-5-pyrazolyl, 1-methyl-4-imidazolyl;
4)

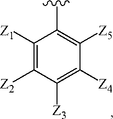

wherein Z₁, Z₂, Z₃, Z₄, Z₅ each are independently selected from the following groups, and Z₁, Z₂, Z₃, Z₄, Z₅ are not hydrogen at the same time:
   (1) H, F, Cl, Br, l, nitro, cyano,
   (2) C₁-C₆ alkyl, C₁-C₆ alkoxy, C₁-C₆ fluorine-containing alkyl, C₁-C₆ fluorine-containing alkoxy, C₂-C₆ alkenyl,
   (3) piperazinyl, N-methylpiperazinyl, N-ethylpiperazinyl, N-n-propylpiperazinyl, N-isopropylpiperazinyl,
   (4) morpholinyl, 2,6-dimethylmorpholinyl, thiomorpholinyl,
   (5) 3-(N,N-dimethylamino)tetrahydropyrrolyl, 3-(N,N-diethylamino)tetrahydropyrrolyl,
   (6) 4-N,N-dimethylaminopiperidyl, 4-N,N-diethylaminopiperidyl, 4-N,N-diisopropylaminopiperidyl, 4-hydroxypiperidyl, 4-(N-methylpiperazinyl-1-)piperidyl, 4-(N-ethylpiperazinyl-1 -)piperidyl, 4-(N-isopropylpiperazinyl-1 -)piperidyl, 4-(tetrahydropyrrolyl-1 -)piperidyl,
   (7) piperidin-4-yl, N-methyl-4-piperidyl, N-ethyl-4-piperidyl, N-isopropyl-4-piperidyl,
   (8) aminoformamido, methylaminoformamido, ethylaminoformamido, propylaminoformamido, isopropylaminoformamido, 2-(N,N-dimethylamino)acetamido, cyclopropylsulfamido, 2-N,N-dimethylaminoethylamino, 3-N,N-dimethylaminopropylamino, 3-N,N-diethylaminopropylamino, 3-N,N-diisopropylaminopropylamino, N-methyl-N-(2-N,N-dimethylamino)ethylamino,
   (9) 4-methyl-1-piperazinylmethylene, 4-ethyl-1-piperazinylmethylene, 4-propyl-1-piperazinylmethylene, 4-isopropyl-1-piperazinylmethylene, benzyl, cyclopropylmethylene, cyclobutylmethylene, cyclopentylmethylene, cyclohexylmethylene,
   (10) 4-(N-methyl-1-piperazinyl)piperidyl-1-formyl, 4-(N-ethyl-1-piperazinyl)piperidyl-1-formyl, N-methylpiperazinyl-1-formyl, N-ethylpiperazinyl-1-formyl, N-isopropylpiperazinyl-1-formyl,
   (11) cyclopropylaminosulfonyl, cyclobutylaminosulfonyl, cyclopentylaminosulfonyl, cyclohexylam inosulfonyl, N-methylpiperazinyl-1-sulfonyl, N-ethylpiperazinyl-1-sulfonyl, N-isopropylpiperazinyl-1-sulfonyl;
5)

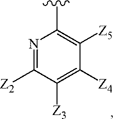

wherein Z₂, Z₃, Z₄, Z₅ each are independently selected from H, C₁-C₆ alkyl, C₁-C₆ alkoxy, piperazinyl, N-methylpiperazinyl, N-ethylpiperazinyl, N-n-propylpiperazinyl, or N-isopropylpiperazinyl, and Z₂, Z₃, Z₄, Z₅ are not hydrogen at the same time;
6)

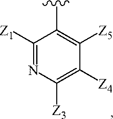

wherein Z₁, Z₃, Z₄, Z₅ each are independently selected from H, C₁—C₆ alkyl, C₁—C₆ alkoxy, N-(N-methyl-3-tetrahydropyrrolyl)amino, N-methyl-N-(N-methyl-3-tetrahydropyrrolyl)amino, N-methylpiperazinyl, N-ethylpiperazinyl, N-n-propylpiperazinyl, N-isopropylpiperazinyl, 4-(N-methylpiperazinyl-1 -)piperidyl, 4-(N-ethylpiperazinyl-1 -)piperidyl, or 4-(N-isopropylpiperazinyl-1-)piperidyl, and Z₁, Z₃, Z₄, Z₅ are not hydrogen at the same time;
7)

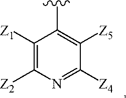

wherein Z₁, Z₂, Z₄, Z₅ each are independently selected from H, or C₁-C₆ alkyl, and Z₁, Z₂, Z₄, Z₅ may be hydrogen at the same time;
8)

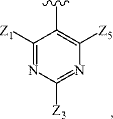

wherein Z₁, Z₃, Z₅ each are independently selected from H, C₁-C₆ alkyl, C₁-C₆ alkoxy, N-methylpiperazinyl, N-ethylpiperazinyl, N-n-propylpiperazinyl, or N-isopropylpiperazinyl, and Z₁, Z₃, Z₅ are not hydrogen at the same time;
9)

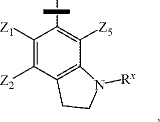

wherein Z₁, Z₂, Z₅ each are independently selected from H, C₁-C₆ alkyl, or C₁-C₆ alkoxy, and Z₁, Z₂, Z₅ may be hydrogen at the same time, R^{X} is selected from aminoformyl, methylaminoformyl, N,N-dimethylaminoformyl, ethylaminoformyl, aminoacetyl, methylaminoacetyl, 2-(N,N-dimethylamino)acetyl, or ethylaminoacetyl;
10)

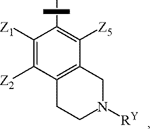

wherein Z₁, Z₂, Z₅ each are independently selected from H, F, Cl, Br, l, C₁-C₆ alkyl, C₁-C₆ alkoxy, C₁-C₆ fluorine-containing alkyl, or C₁-C₆ fluorine-containing alkoxy, and Z₁, Z₂, Z₅ may be hydrogen at the same time, R^{y} is C₁-C₆ alkyl;
11)

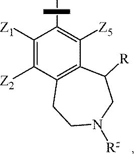

wherein Z₁, Z₂, Z₅ each are independently selected from H, or C₁-C₆ alkyl, and Z₁, Z₂, Z₅ may be hydrogen at the same time, R^{z}, R each are independently selected from C₁-C₆ alkyl.

In some embodiments, R¹ is selected from:
1) cyclopropyl, 2-N,N-dimethylaminoethyl, 2-hydroxyethyl, N-methyl-4-piperidyl, 4-aminocyclohexyl, 1-methyl-3-pyrazolyl, 1-methyl-4-pyrazolyl, 1,3-dimethyl-5-pyrazolyl, 1-methyl-4-imidazolyl;
2)

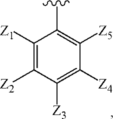

wherein: Z₁, Z₂, Z₃, Z₄, Z₅ each are independently selected from the following groups, and Z₁, Z₂, Z₃, Z₄, Z₅ are not hydrogen at the same time:
   H, methyl, ethyl, methoxy, ethoxy, isopropoxy, trifluoromethyl, trifluoromethoxy, vinyl, N-methylpiperazinyl, morpholinyl, 3-(N,N-dimethylamino)tetrahydropyrrolyl, 4-N,N-dimethylaminopiperidyl, 4-hydroxypiperidyl, 4-(N-methylpiperazinyl-1-)piperidyl, 4-(tetrahydropyrrolyl-1-)piperidyl, N-methyl-4-piperidyl, 2-(N,N-dimethylamino)acetamido, cyclopropylsulfamido, N-methyl-N-(2-N,N-dimethylamino)ethylamino, 4-methyl-1-piperazinylmethylene, benzyl, cyclopropylmethylene, 4-(N-methyl-1-piperazinyl)piperidyl-1-formyl, N-methylpiperazinyl-1-formyl, cyclopropylaminosulfonyl, or N-methylpiperazinyl-1-sulfonyl;
   preferably, Z₁, Z₂, Z₅ each are independently selected from H, methoxy, ethoxy, isopropoxy, trifluoromethyl, trifluoromethoxy; and one of Z₃ and Z₄ is selected from the following groups, and the other is H, methyl, ethyl, vinyl, benzyl or cyclopropylmethylene: N-methylpiperazinyl, morpholinyl, 3-(N,N-dimethylamino)tetrahydropyrrolyl, 4-N,N-dimethylaminopiperidyl, 4-hydroxypiperidyl, 4-(N-methylpiperazinyl-1-)piperidyl, 4-(tetrahydropyrrolyl-1-)piperidyl, N-methyl-4-piperidyl, 2-(N,N-dimethylamino)acetamido, cyclopropylsulfamido, N-methyl-N-(2-N,N-dimethylamino)ethylamino, 4-methyl-1-piperazinylmethylene, 4-(N-methyl-1-piperazinyl)piperidyl-1-formyl, N-methylpiperazinyl-1-formyl, cyclopropylaminosulfonyl, or N-methylpiperazinyl-1-sulfonyl;
3)

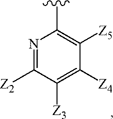

wherein Z₂, Z₃, Z₄, Z₅ each are independently selected from H, methoxy, or N-methylpiperazinyl, and Z₂, Z₃, Z₄, Z₅ are not hydrogen at the same time;
4)

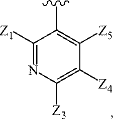

wherein:
   Z₁, Z₃, Z₄, Z₅ each are independently selected from H, methyl, ethyl, methoxy, N-(N-methyl-3-tetrahydropyrrolyl)am ino, N-methyl-N-(N-methyl-3-tetrahydropyrrolyl)amino, N-methylpiperazinyl, or 4-(N-methylpiperazinyl-1-)piperidyl, and Z₁, Z₃, Z₄, Z₅ are not hydrogen at the same time;
   preferably, Z₁, Z₄, Z₅ each are independently selected from H, methyl, ethyl, or methoxy, Z₃ is methyl, N-(N-methyl-3-tetrahydropyrrolyl)amino, N-methyl-N-(N-methyl-3-tetrahydropyrrolyl)amino, N-methylpiperazinyl or 4-(N-methylpiperazinyl-1-)piperidyl;
5)

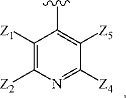

wherein Z₁, Z₂, Z₄, Z₅ are H;
6)

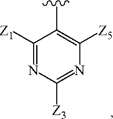

wherein Z₁, Z₃, Z₅ each are independently selected from H methoxy, or N-methylpiperazinyl, and Z₁, Z₃, Z₅ are not hydrogen at the same time;
7)

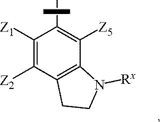

wherein Z₁, Z₂, Z₅ each are independently selected from H, or methoxy, R^{X} is 2-(N,N-dimethylamino)acetyl;
8)

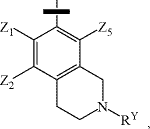

wherein Z₁, Z₂, Z₅ each are independently selected from H, F, Cl, methyl, methoxy, trifluoromethyl, or trifluoromethoxy, R^{y} is methyl;
9)

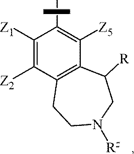

wherein Z₁, Z₂, Z₅ are H, R^{z}, R are methyl.

In some embodiments, R², R³ each are independently selected from C₁-C₆ alkyl, C₁-C₆ fluorine-containing alkyl, or C₃-C₆ cycloalkyl.

In some embodiments, R², R³ each are independently selected from methyl, trifluoromethyl, ethyl, isopropyl, or cyclopropyl.

In some embodiments, R² is selected from methyl, trifluoromethyl, ethyl, isopropyl, or cyclopropyl, R³ is selected from methyl, ethyl, isopropyl, or cyclopropyl.

In some embodiments, R⁵ is selected from F, Cl, Br, trifluoromethyl, cyano, nitro, methyl, methoxy, or cyclopropyl.

In the sixth aspect of the present invention, the present invention provides a compound having the general formula (II-1) or (II-2):

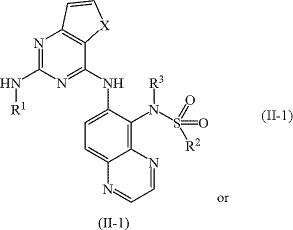

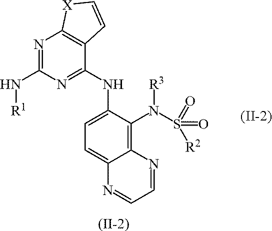

or a stereoisomer, a prodrug, a pharmaceutically acceptable salt or a pharmaceutically acceptable solvate thereof, wherein:
each X is independently selected from NH, O, or S;
each R¹ is independently selected from:
   1) 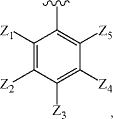
   wherein Z₁, Z₂, Z₃, Z₄, Z₅ each are independently selected from the following groups, and Z₁, Z₂, Z₃, Z₄, Z₅ are not hydrogen at the same time:
      (1) H, F, Cl, Br, l, nitro, cyano,
      (2) C₁-C₆ alkyl, C₁-C₆ alkoxy, C₁-C₆ oxygen-containing alkyl, C₁-C₆ fluorine-containing alkyl, C₁-C₆ fluorine-containing alkoxy, C₃-C₆ cycloalkyl, C₂-C₆ alkenyl,
      (3) 2-N,N-dimethylaminoethoxy, 3-N,N-dimethylaminopropoxy, 2-(N-methylpiperazinyl)ethoxy, 2-(N-acetylpiperazinyl)ethoxy, 2-morpholinylethoxy, 2-thiomorpholinylethoxy, 2-piperidylethoxy, 3-N,N-dimethylaminopropoxy, 3-N,N-diethylaminopropoxy, 3-N,N-diisopropylaminopropoxy, 3-(N-methylpiperazinyl)propoxy, 3-(N-acetylpiperazinyl)propoxy, 3-morpholinylpropoxy, 3-thiomorpholinylpropoxy, 3-piperidylpropoxy, 2-pyridylmethoxy, 3-pyridylmethoxy, 4-pyridylmethoxy, phenylmethoxy, monohalo-substituted phenylmethoxy, gem-dihalo-substituted phenylmethoxy, hetero-dihalo-substituted phenylmethoxy,
      (4) piperazinyl, N-methylpiperazinyl, N-ethylpiperazinyl, N-n-propylpiperazinyl, N-isopropylpiperazinyl, N-cyclopropylpiperazinyl, N-(2-hydroxyethyl)piperazinyl, N-(2-N,N-dimethylaminoethyl)piperazinyl, N-(2-N,N-diethylaminoethyl)piperazinyl, N-(2-cyanoethyl)piperazinyl, N-cyanomethylpiperazinyl, N-(3-hydroxypropyl)piperazinyl, N-(3-N,N-dimethylaminopropyl)piperazinyl, N-(3-N,N-diethylaminopropyl)piperazinyl, N-(N-methyl-4-piperidyl)piperazinyl, N-(N-ethyl-4-piperidyl)piperazinyl, N-t-butoxycarbonylpiperazinyl, N-acetylpiperazinyl, N-propionylpiperazinyl, N-isobutyrylpiperazinyl, N-cyclopropylformylpiperazinyl, N-methylsulfonylpiperazinyl, N-ethylsulfonylpiperazinyl, N-n-propylsulfonylpiperazinyl, N-isopropylsulfonylpiperazinyl, N-cyclopropylsulfonylpiperazinyl, 2-oxo-piperazin-4-yl,
      (5) morpholinyl, 2,6-dimethylmorpholinyl, thiomorpholinyl,
      (6) tetrahydropyrrolyl, 3-(N,N-dimethylamino)tetrahydropyrrolyl, 3-(N, N-diethylam ino)tetrahydropyrrolyl,
      (7) imidazolyl, 4-methyl-1-imidazolyl,
      (8) piperidyl, 4-N,N-dimethylaminopiperidyl, 4-N,N-diethylaminopiperidyl, 4-N,N-diisopropylaminopiperidyl, 4-hydroxypiperidyl, 4-methoxypiperidyl, 4-ethoxypiperidyl, 4-(N-methylpiperazinyl-1-)piperidyl, 4-(N-ethylpiperazinyl-1-)piperidyl, 4-(N-isopropylpiperazinyl-1-)piperidyl, 4-(N-acetylpiperazinyl-1-)piperidyl, 4-(N-t-butoxycarbonylpiperazinyl-1-)piperidyl, 4-(N-methylsulfonylpiperazinyl-1-)piperidyl, 4-(N-(2-hydroxyethyl)piperazinyl-1-)piperidyl, 4-(N-(2-cyanoethyl)piperazinyl-1-)piperidyl, 4-(N-(3-hydroxypropyl)piperazinyl-1-)piperidyl, 4-(N-(2-N, N-dimethylam inoethyl)piperazinyl-1 -)piperidyl, 4-(N-(2-N,N-diethylethyl)piperazinyl-1 -)piperidyl, 4-(N-(3-N,N-dimethylpropyl)piperazinyl-1 -)piperidyl, 4-(N-(3-N,N-diethylpropyl)piperazinyl-1-)piperidyl, 4-(tetrahydropyrrolyl-1-)piperidyl, 4-(3-N, N-dimethylam inotetrahydropyrrolyl-1 -)piperidyl, 4-(3-N, N-diethylam inotetrahydropyrrolyl-1 -)piperidyl,
      (9) piperidin-4-yl, N-methyl-4-piperidyl, N-ethyl-4-piperidyl, N-isopropyl-4-piperidyl, N-acetyl-4-piperidyl, N-t-butoxycarbonyl-4-piperidyl, N-methylsulfonyl-4-piperidyl, N-(2-hydroxyethyl)-4-piperidyl, N-(2-N, N-dimethylam inoethyl)-4-piperidyl, N-(2-N, N-diethylam inoethyl)-4-piperidyl, N-(2-cyanoethyl)-4-piperidyl, N-(3-hydroxypropyl)-4-piperidyl, N-(3-N, N-dimethylam inopropyl)-4-piperidyl, N-(3-N, N-diethylam inopropyl)-4-piperidyl, N-(3-cyanopropyl)-4-piperidyl, N-cyanomethylene-4-piperidyl,
      (10) amino, N,N-dimethylamino, N,N-diethylamino, N,N-di-n-propylamino, N,N-diisopropylamino, 2-N,N-dimethylaminoethylamino, 2-morpholinylethylamino, 2-(N-methylpiperazinyl)ethylam ino, 3-N, N-dimethylaminopropylam ino, 3-N, N-diethylam inopropylam ino, 3-N, N-diisopropylaminopropylam ino, 3-morpholinylpropylamino, 3-(N-methylpiperazinyl)propylamino, N-methylpiperidyl-4-amino, N-ethylpiperidyl-4-amino, N-n-propylpiperidyl-4-amino, N-isopropylpiperidyl-4-am ino, acetamido, propionamido, 2-(N,N-dimethylamino)acetamido, 2-hydroxyacetamido, 2-methoxyacetamido, methylsulfamido, ethylsulfamido, n-propylsulfamido, isopropylsulfamido, cyclopropylsulfam ido, N-methyl-N-(N-methyl-3-tetrahydropyrrolyl)am ino, N-(N-methyl-3-tetrahydropyrrolyl)amino, N-methyl-N-(2-N,N-dimethylamino)ethylamino,
      (11) aminomethylene, N,N-dimethylaminomethylene, N,N-diethylaminomethylene, 4-methyl-1-piperazinylmethylene, 4-ethyl-1-piperazinylmethylene, 4-propyl-1-piperazinylmethylene, 4-isopropyl-1-piperazinylmethylene, 4-(2-hydroxyethyl)-1-piperazinylmethylene, 4-(2-N,N-dimethylaminoethyl)-1-piperazinylmethylene, piperidyl-1-methylene, 4-(N,N-dimethylamino)-1-piperidylmethylene, 4-hydroxypiperidyl-1-methylene, tetrahydropyrrolyl-1-methylene, 3-(N,N-dimethylamino)-1-tetrahydropyrrolylmethylene, benzyl, cyclopropylmethylene, cyclobutylmethylene, cyclopentylmethylene, cyclohexylmethylene,
      (12) aminoformyl, methylaminoformyl, N,N-dimethylaminoformyl, ethylaminoformyl, cyclopropylaminoformyl, cyclobutylaminoformyl, cyclopentylaminoformyl, cyclohexylaminoformyl, aminoacetyl, methylaminoacetyl, 2-(N,N-dimethylamino)acetyl, ethylaminoacetyl, cyclopropylaminoacetyl, cyclobutylaminoacetyl, cyclopentylaminoacetyl, cyclohexylaminoacetyl, tetrahydropyrrolyl-1-formyl, 3-(N,N-dimethylamino)tetrahydropyrrolyl-1-formyl, 3-(N,N-diethylamino)tetrahydropyrrolyl-1-formyl, morpholinyl-4-formyl, thiomorpholinyl-4-formyl, 2,6-dimethylmorpholinyl-4-formyl, piperidyl-1-formyl, 4-hydroxypiperidyl-1-formyl, 4-(N,N-dimethylamino)piperidyl-1-formyl, 4-(N,N-diethylamino)piperidyl-1-formyl, 4-(N-methyl-1-piperazinyl)piperidyl-1-formyl, 4-(N-ethyl-1-piperazinyl)piperidyl-1-formyl, N-methylpiperazinyl-1-formyl, N-ethylpiperazinyl-1-formyl, N-isopropylpiperazinyl-1-formyl, N-(2-hydroxyethyl)piperazinyl-1-formyl, N-(2-N,N-dimethylaminoethyl)piperazinyl-1-formyl, N-(2-N,N-diethylaminoethyl)piperazinyl-1-formyl, 4-(N-methyl-4-piperidyl)piperazinyl-1-formyl, 4-(N-ethyl-4-piperidyl)piperazinyl-1-formyl,
      (13) hydroxyformyl, methoxyformyl, ethoxyformyl, propoxyformyl, isopropoxyformyl, n-butoxyformyl, iso-butoxyformyl, t-butoxyformyl,
      (14) hydroxysulfonyl, aminosulfonyl, methylaminosulfonyl, ethylaminosulfonyl, propylaminosulfonyl, isopropylaminosulfonyl, cyclopropylaminosulfonyl, cyclobutylaminosulfonyl, cyclopentylaminosulfonyl, cyclohexylaminosulfonyl, tetrahydropyrrolyl-1-sulfonyl, 3-(N,N-dimethylamino)tetrahydropyrrolyl-1-sulfonyl, 3-(N,N-diethylamino)tetrahydropyrrolyl-1-sulfonyl, morpholinyl-4-sulfonyl, thiomorpholinyl-4-sulfonyl, 2,6-dimethylmorpholinyl-4-sulfonyl, piperidyl-1-sulfonyl, 4-hydroxypiperidyl-1-sulfonyl, 4-(N,N-dimethylamino)piperidyl-1-sulfonyl, 4-(N,N-diethylamino)piperidyl-1-sulfonyl, 4-(N-methyl-1-piperazinyl)piperidyl-1-sulfonyl, 4-(N-ethyl-1-piperazinyl)piperidyl-1-sulfonyl, N-methylpiperazinyl-1-sulfonyl, N-ethylpiperazinyl-1-sulfonyl, N-isopropylpiperazinyl-1-sulfonyl, N-(2-hydroxyethyl)piperazinyl-1-sulfonyl, N-(2-N,N-dimethylaminoethyl)piperazinyl-1-sulfonyl, N-(2-N,N-diethylaminoethyl)piperazinyl-1-sulfonyl, 4-(N-methyl-4-piperidyl)piperazinyl-1-sulfonyl, 4-(N-ethyl-4-piperidyl)piperazinyl-1-sulfonyl,
      (15) aminoformamido, methylaminoformamido, ethylaminoformamido, propylaminoformamido, isopropylaminoformamido, cyclopropylaminoformamido, cyclobutylaminoformamido, cyclopentylaminoformamido, piperidyl-1-formamido, 4-hydroxypiperidyl-1-formamido, 4-N,N-dimethylpiperidyl-1-formamido, 4-N,N-diethylpiperidyl-1-formamido, tetrahydropyrrolyl-1-formamido, 3-N,N-dimethyltetrahydropyrrolyl-1-formam ido, 3-N,N-diethyltetrahydropyrrolyl-1-formamido, N-methylpiperazinyl-1-formamido, N-ethylpiperazinyl-1-formamido, N-acetylpiperazinyl-1-formamido, N-t-butoxycarbonylpiperazinyl-1-formamido, N-(2-hydroxyethyl)piperazinyl-1-formamido, N-(2-cyanoethyl)piperazinyl-1-formam ido, N-(2-N,N-dimethylaminoethyl)piperazinyl-1-formamido, N-(2-N,N-diethylaminoethyl)piperazinyl-1-formamido, N-(3-hydroxypropyl)piperazinyl-1-formamido, N-(3-N,N-dimethylpropyl)piperazinyl-1-formamido, N-(3-N,N-diethylpropyl)piperazinyl-1-formamido, morpholinyl-1-formamido, 3,5-dimethylmorpholinyl-1-formamido, 4-(tetrahydropyrrolyl)piperidyl-1-formamido, 4-(N-methyl-1-piperazinyl)piperidyl-1-formamido, 4-(N-ethyl-1-piperazinyl)piperidyl-1-formamido, 4-(N-acetyl-1-piperazinyl)piperidyl-1-formamido, N-(N-methyl-4-piperidyl)piperazinyl-1-formamido;
   2) 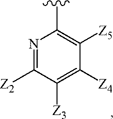
   wherein Z₂, Z₃, Z₄, Z₅ are the same as defined in 1), and Z₂, Z₃, Z₄, Z₅ are not hydrogen at the same time;
   3) 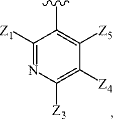
   wherein Z₁, Z₃, Z₄, Z₅ are the same as defined in 1), and Z₁, Z₃, Z₄, Z₅ are not hydrogen at the same time;
   4) 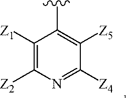
   wherein Z₁, Z₂, Z₄, Z₅ are the same as defined in 1), and Z₁, Z₂, Z₄, Z₅ may be hydrogen at the same time;
   5) 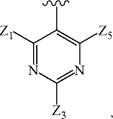
   wherein Z₁, Z₃, Z₅ are the same as defined in 1), and Z₁, Z₃, Z₅ are not hydrogen at the same time;
R², R³ each are independently selected from:
   H, C₁-C₆ alkyl, C₁-C₆ fluorine-containing alkyl, C₃-C₆ cycloalkyl, or C₃-C₆ fluorine-containing cycloalkyl.

In some embodiments, each R¹ is independently selected from:
1)

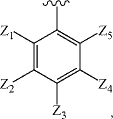

Z₁, Z₂, Z₃, Z₄, Z₅ each are independently selected from the following groups, and Z₁, Z₂, Z₃, Z₄, Z₅ are not hydrogen at the same time:
   (1) H, F, Cl, Br, I, nitro, cyano,
   (2) C₁-C₆ alkyl, C₁-C₆ alkoxy, C₁-C₆ oxygen-containing alkyl, C₁-C₆ fluorine-containing alkyl, C₁-C₆ fluorine-containing alkoxy, C₃-C₆ cycloalkyl, C₂-C₆ alkenyl,
   (3) piperazinyl, N-methylpiperazinyl, N-ethylpiperazinyl, N-n-propylpiperazinyl, N-isopropylpiperazinyl, N-cyclopropylpiperazinyl, N-(2-hydroxyethyl)piperazinyl, N-(2-N,N-dimethylaminoethyl)piperazinyl, N-(2-N,N-diethylaminoethyl)piperazinyl, N-(2-cyanoethyl)piperazinyl, N-cyanomethylpiperazinyl, N-(3-hydroxypropyl)piperazinyl, N-(3-N,N-dimethylaminopropyl)piperazinyl, N-(3-N,N-diethylaminopropyl)piperazinyl,
   (4) morpholinyl, 2,6-dimethylmorpholinyl, thiomorpholinyl,
   (5) tetrahydropyrrolyl, 3-(N,N-dimethylamino)tetrahydropyrrolyl, 3-(N,N-diethylamino)tetrahydropyrrolyl,
   (6) imidazolyl, 4-methyl-1-imidazolyl,
   (7) piperidyl, 4-N,N-dimethylaminopiperidyl, 4-N,N-diethylaminopiperidyl, 4-N,N-diisopropylaminopiperidyl, 4-hydroxypiperidyl, 4-methoxypiperidyl, 4-ethoxypiperidyl, 4-(N-methylpiperazinyl-1-)piperidyl, 4-(N-ethylpiperazinyl-1-)piperidyl, 4-(N-isopropylpiperazinyl-1-)piperidyl, 4-(N-(2-hydroxyethyl)piperazinyl-1-)piperidyl, 4-(N-(2-cyanoethyl)piperazinyl-1-)piperidyl, 4-(N-(3-hydroxypropyl)piperazinyl-1-)piperidyl, 4-(N-(2-N,N-dimethylaminoethyl)piperazinyl-1-)piperidyl, 4-(N-(2-N,N-diethylethyl)piperazinyl-1 -)piperidyl, 4-(N-(3-N,N-dimethylpropyl)piperazinyl-1-)piperidyl, 4-(N-(3-N,N-diethylpropyl)piperazinyl-1-)piperidyl, 4-(tetrahydropyrrolyl-1-)piperidyl, 4-(3-N,N-dimethylaminotetrahydropyrrolyl-1-)piperidyl, 4-(3-N,N-diethylaminotetrahydropyrrolyl-1-)piperidyl,
   (8) piperidin-4-yl, N-methyl-4-piperidyl, N-ethyl-4-piperidyl, N-isopropyl-4-piperidyl, N-(2-hydroxyethyl)-4-piperidyl, N-(2-N,N-dimethylaminoethyl)-4-piperidyl, N-(2-N,N-diethylaminoethyl)-4-piperidyl, N-(2-cyanoethyl)-4-piperidyl, N-(3-hydroxypropyl)-4-piperidyl, N-(3-N,N-dimethylaminopropyl)-4-piperidyl, N-(3-N,N-diethylaminopropyl)-4-piperidyl, N-(3-cyanopropyl)-4-piperidyl, N-cyanomethylene-4-piperidyl,
   (9) amino, N,N-dimethylamino, N,N-diethylamino, N,N-di-n-propylamino, N,N-diisopropylamino, 2-N,N-dimethylaminoethylamino, 3-N,N-dimethylaminopropylamino, 3-N,N-diethylaminopropylamino, 3-N,N-diisopropylaminopropylamino, N-methylpiperidyl-4-amino, N-ethylpiperidyl-4-amino, N-n-propylpiperidyl-4-amino, N-isopropylpiperidyl-4-amino, acetamido, propionamido, 2-(N,N-dimethylamino)acetamido, 2-hydroxyacetamido, 2-methoxyacetamido, methylsulfamido, ethylsulfamido, n-propylsulfamido, isopropylsulfamido, cyclopropylsulfamido, N-methyl-N-(N-methyl-3-tetrahydropyrrolyl)amino, N-(N-methyl-3-tetrahydropyrrolyl)amino, N-methyl-N-(2-N,N-dimethylamino)ethylamino,
   (10) 4-methyl-1-piperazinylmethylene, 4-ethyl-1-piperazinylmethylene, 4-propyl-1-piperazinylmethylene, 4-isopropyl-1-piperazinylmethylene, 4-(2-hydroxyethyl)-1-piperazinylmethylene, 4-(2-N,N-dimethylaminoethyl)-1-piperazinylmethylene, piperidyl-1-methylene, 4-(N,N-dimethylamino)-1-piperidylmethylene, 4-hydroxypiperidyl-1-methylene, tetrahydropyrrolyl-1-methylene, 3-(N,N-dimethylamino)-1-tetrahydropyrrolylmethylene, benzyl, cyclopropylmethylene, cyclobutylmethylene, cyclopentylmethylene, cyclohexylmethylene,
   (11) aminoformyl, methylaminoformyl, N,N-dimethylaminoformyl, ethylaminoformyl, aminoacetyl, methylaminoacetyl, 2-(N,N-dimethylamino)acetyl, ethylaminoacetyl, tetrahydropyrrolyl-1-formyl, 3-(N,N-dimethylamino)tetrahydropyrrolyl-1-formyl, 3-(N,N-diethylamino)tetrahydropyrrolyl-1-formyl, morpholinyl-4-formyl, thiomorpholinyl-4-formyl, 2,6-dimethylmorpholinyl-4-formyl, piperidyl-1-formyl, 4-hydroxypiperidyl-1-formyl, 4-(N,N-dimethylamino)piperidyl-1-formyl, 4-(N,N-diethylamino)piperidyl-1-formyl, 4-(N-methyl-1-piperazinyl)piperidyl-1-formyl, 4-(N-ethyl-1-piperazinyl)piperidyl-1-formyl, N-methylpiperazinyl-1-formyl, N-ethylpiperazinyl-1-formyl, N-isopropylpiperazinyl-1-formyl, N-(2-hydroxyethyl)piperazinyl-1-formyl, N-(2-N,N-dimethylaminoethyl)piperazinyl-1-formyl, N-(2-N,N-diethylaminoethyl)piperazinyl-1-formyl, 4-(N-methyl-4-piperidyl)piperazinyl-1-formyl, 4-(N-ethyl-4-piperidyl)piperazinyl-1-formyl,
   (12) hydroxysulfonyl, aminosulfonyl, methylaminosulfonyl, ethylaminosulfonyl, propylaminosulfonyl, isopropylaminosulfonyl, cyclopropylaminosulfonyl, cyclobutylaminosulfonyl, cyclopentylaminosulfonyl, cyclohexylaminosulfonyl, tetrahydropyrrolyl-1-sulfonyl, 3-(N,N-dimethylamino)tetrahydropyrrolyl-1-sulfonyl, 3-(N,N-diethylamino)tetrahydropyrrolyl-1-sulfonyl, morpholinyl-4-sulfonyl, thiomorpholinyl-4-sulfonyl, 2,6-dimethylmorpholinyl-4-sulfonyl, piperidyl-1-sulfonyl, 4-hydroxypiperidyl-1-sulfonyl, 4-(N,N-dimethylamino)piperidyl-1-sulfonyl, 4-(N,N-diethylamino)piperidyl-1-sulfonyl, N-methylpiperazinyl-1-sulfonyl, N-ethylpiperazinyl-1-sulfonyl, N-isopropylpiperazinyl-1-sulfonyl, N-(2-hydroxyethyl)piperazinyl-1-sulfonyl, N-(2-N,N-dimethylaminoethyl)piperazinyl-1-sulfonyl, N-(2-N,N-diethylaminoethyl)piperazinyl-1-sulfonyl,
   (13) aminoformamido, methylaminoformamido, ethylaminoformamido, propylaminoformamido, isopropylaminoformamido;
2)

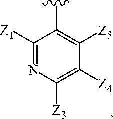

wherein Z₁, Z₃, Z₄, Z₅ are the same as defined in 1), and Z₁, Z₃, Z₄, Z₅ are not hydrogen at the same time.

In some embodiments, each R¹ is independently selected from:
1)

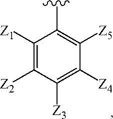

wherein Z₁, Z₂, Z₃, Z₄, Z₅ each are independently selected from the following groups, and Z₁, Z₂, Z₃, Z₄, Z₅ are not hydrogen at the same time:
   (1) H, F, Cl, Br, I, nitro, cyano,
   (2) C₁-C₆ alkyl, C₁-C₆ alkoxy, C₁-C₆ fluorine-containing alkoxy, C₂-C₆ alkenyl,
   (3) piperazinyl, N-methylpiperazinyl, N-ethylpiperazinyl, N-n-propylpiperazinyl, N-isopropylpiperazinyl,
   (5) 3-(N,N-dimethylamino)tetrahydropyrrolyl, 3-(N,N-diethylamino)tetrahydropyrrolyl,
   (6) 4-N,N-dimethylaminopiperidyl, 4-N,N-diethylaminopiperidyl, 4-N,N-diisopropylaminopiperidyl, 4-(N-methylpiperazinyl-1-)piperidyl, 4-(N-ethylpiperazinyl-1-)piperidyl, 4-(N-isopropylpiperazinyl-1-)piperidyl,
   (7) benzyl, cyclopropylmethylene, cyclobutylmethylene, cyclopentylmethylene, cyclohexylmethylene,
2)

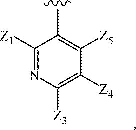

wherein Z₁, Z₃, Z₄, Z₅ each are independently selected from H, C₁-C₆ alkyl, C₁-C₆ alkoxy, 4-(N-methylpiperazinyl-1-)piperidyl, 4-(N-ethylpiperazinyl-1-)piperidyl, or 4-(N-isopropylpiperazinyl-1-)piperidyl, and Z₁, Z₃, Z₄, Z₅ are not hydrogen at the same time.

In some embodiments, each R¹ is independently selected from:
1)

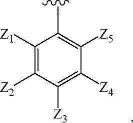

wherein: Z₁, Z₂, Z₃, Z₄, Z₅ each are independently selected from the following groups, and Z₁, Z₂, Z₃, Z₄, Z₅ are not hydrogen at the same time:
   H, methyl, ethyl, methoxy, trifluoromethoxy, vinyl, N-methylpiperazinyl, 3-(N,N-dimethylamino)tetrahydropyrrolyl, 4-N,N-dimethylaminopiperidyl, 4-(N-methylpiperazinyl-1-)piperidyl, benzyl, or cyclopropylmethylene;
   preferably, Z₁, Z₂, Z₅ each are independently selected from H, methoxy, trifluoromethoxy, Z₄ is H, methyl, ethyl, vinyl, benzyl or cyclopropylmethylene, Z₃ is N-methylpiperazinyl, 3-(N,N-dimethylamino)tetrahydropyrrolyl, 4-N,N-dimethylaminopiperidyl or 4-(N-methylpiperazinyl-1-)piperidyl;
2)

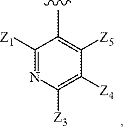

wherein: Z₁, Z₃, Z₄, Z₅ each are independently selected from H, methyl, ethyl, methoxy, or 4-(N-methylpiperazinyl-1-)piperidyl, and Z₁, Z₃, Z₄, Z₅ are not hydrogen at the same time; preferably, Z₁, Z₄, Z₅ each are independently selected from H, methyl, ethyl, or methoxy, Z₃ is 4-(N-methylpiperazinyl-1-)piperidyl.

In some embodiments, R², R³ each are independently selected from H, or C₁-C₆ alkyl.

In some embodiments, R², R³ each are independently selected from methyl.

In the seventh aspect of the present invention, the present invention provides a compound having the general formula (A):

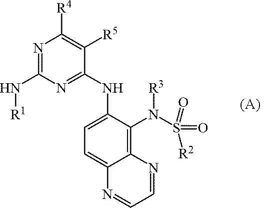

or a stereoisomer, a prodrug, a pharmaceutically acceptable salt or a pharmaceutically acceptable solvate thereof, wherein:
R¹ is selected from:
   1) H, C₁-C₆ alkyl, C₁-C₆ fluorine-containing alkyl, C₃-C₈ cycloalkyl, C₁-C₆ heteroatom-containing alkyl, C₃-C₈ heteroatom-containing cycloalkyl;
   2) 2-N,N-dimethylaminoethyl, 3-N,N-dimethylaminopropyl, 2-N,N-diethylaminoethyl, 3-N,N-diethylaminopropyl, 2-(N-methylpiperazinyl)ethyl, 2-(N-acetylpiperazinyl)ethyl, 2-morpholinylethyl, 2-thiomorpholinylethyl,2-piperidylethyl, 2-hydroxyethyl, 3-hydroxypropyl, 2-methoxyethyl, N-methyl-4-piperidyl, N-ethyl-4-piperidyl, N-propyl-4-piperidyl, N-isopropyl-4-piperidyl, N-hydroxyethyl-4-piperidyl, N-cyanomethyl-4-piperidyl, N-(2-cyanoethyl)-4-piperidyl, N-acetyl-4-piperidyl, N-propionyl-4-piperidyl, N-isopropionyl-4-piperidyl, N-cyclopropylformyl-4-piperidyl, N-methylsulfonyl-4-piperidyl, N-ethylsulfonyl-4-piperidyl, N-propylsulfonyl-4-piperidyl, N-isopropylsulfonyl-4-piperidyl, N-cyclopropylsulfonyl-4-piperidyl, 3-N,N-dimethylaminocyclopentyl, 3-N,N-diethylaminocyclopentyl, 3-N,N-dipropylaminocyclopentyl, 3-N,N-isopropylaminocyclopentyl, 4-aminocyclohexyl, 4-N,N-dimethylaminocyclohexyl, 4-N,N-diethylaminocyclohexyl, 4-N,N-dipropylaminocyclohexyl, 4-N,N-diisopropylaminocyclohexyl, 4-acetylaminocyclohexyl, 4-propionylaminocyclohexyl, 4-isopropylformylaminocyclohexyl, 4-methylsulfonylaminocyclohexyl, 4-ethylsulfonylaminocyclohexyl, 4-propylsulfonylaminocyclohexyl, 4-isopropylsulfonylaminocyclohexyl, 4-cyclopropylsulfonylaminocyclohexyl, 4-hydroxycyclohexyl, 4-methoxycyclohexyl, 4-ethoxycyclohexyl, 4-isopropoxycyclohexyl;
   3)1-methyl-3-pyrrolyl, 1-ethyl-3-pyrrolyl, 1-isopropyl-3-pyrrolyl, 1-cyclopropyl-3-pyrrolyl, 1-methyl-3-pyrazolyl, 1-ethyl-3-pyrazolyl, 1-isopropyl-3-pyrazolyl, 1-cyclopropyl-3-pyrazolyl, 1-hydroxyethyl-3-pyrazolyl, 1-methyl-4-pyrazolyl, 1-ethyl-4-pyrazolyl, 1-isopropyl-4-pyrazolyl, 1-cyclopropyl-4-pyrazolyl, 1-hydroxyethyl-4-pyrazolyl, 1-(N-methyl-4-piperidyl)-4-pyrazolyl, 1-(N-t-butoxycarbonyl-4-piperidyl)-4-pyrazolyl, 1,3-dimethyl-5-pyrazolyl, 1-methl-4-imidazolyl, 3-methyl-5-isoxazolyl;
   4) 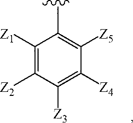
   wherein Z₁, Z₂, Z₃, Z₄, Z₅ each are independently selected from the following groups, and Z₁, Z₂, Z₃, Z₄, Z₅ are not hydrogen at the same time:
      (1) H, F, Cl, Br, I, nitro, cyano,
      (2) C₁-C₆ alkyl, C₁-C₆ alkoxy, C₁-C₆ oxygen-containing alkyl, C₁-C₆ fluorine-containing alkyl, C₁-C₆ fluorine-containing alkoxy, C₃-C₆ cycloalkyl, C₂-C₆ alkenyl,
      (3) 2-N,N-dimethylaminoethoxy, 3-N,N-dimethylaminopropoxy, 2-(N-methylpiperazinyl)ethoxy, 2-(N-acetylpiperazinyl)ethoxy, 2-morpholinylethoxy, 2-thiomorpholinylethoxy, 2-piperidylethoxy, 3-N,N-dimethylaminopropoxy, 3-N,N-diethylaminopropoxy, 3-N,N-diisopropylaminopropoxy, 3-(N-methylpiperazinyl)propoxy, 3-(N-acetylpiperazinyl)propoxy, 3-morpholinylpropoxy, 3-thiomorpholinylpropoxy, 3-piperidylpropoxy, 2-pyridylmethoxy, 3-pyridylmethoxy, 4-pyridylmethoxy, phenylmethoxy, monohalo-substituted phenylmethoxy, gem-dihalo-substituted phenylmethoxy, hetero-dihalo-substituted phenylmethoxy,
      (4) piperazinyl, N-methylpiperazinyl, N-ethylpiperazinyl, N-n-propylpiperazinyl, N-isopropylpiperazinyl, N-cyclopropylpiperazinyl, N-(2-hydroxyethyl)piperazinyl, N-(2-N,N-dimethylaminoethyl)piperazinyl, N-(2-N,N-diethylaminoethyl)piperazinyl, N-(2-cyanoethyl)piperazinyl, N-cyanomethylpiperazinyl, N-(3-hydroxypropyl)piperazinyl, N-(3-N,N-dimethylaminopropyl)piperazinyl, N-(3-N,N-diethylaminopropyl)piperazinyl, N-(N-methyl-4-piperidyl)piperazinyl, N-(N-ethyl-4-piperidyl)piperazinyl, N-t-butoxycarbonylpiperazinyl, N-acetylpiperazinyl, N-propionylpiperazinyl, N-isobutyrylpiperazinyl, N-cyclopropylformylpiperazinyl, N-methylsulfonylpiperazinyl, N-ethylsulfonylpiperazinyl, N-n-propylsulfonylpiperazinyl, N-isopropylsulfonylpiperazinyl, N-cyclopropylsulfonylpiperazinyl, 2-oxo-piperazin-4-yl,
      (5) morpholinyl, 2,6-dimethylmorpholinyl, thiomorpholinyl,
      (6) tetrahydropyrrolyl, 3-(N,N-dimethylamino)tetrahydropyrrolyl, 3-(N,N-diethylamino)tetrahydropyrrolyl,
      (7) imidazolyl, 4-methyl-1-imidazolyl,
      (8) piperidyl, 4-N,N-dimethylaminopiperidyl, 4-N,N-diethylaminopiperidyl, 4-N,N-diisopropylaminopiperidyl, 4-hydroxypiperidyl, 4-methoxypiperidyl, 4-ethoxypiperidyl, 4-(N-methylpiperazinyl-1-)piperidyl, 4-(N-ethylpiperazinyl-1-)piperidyl, 4-(N-isopropylpiperazinyl-1-)piperidyl, 4-(N-acetylpiperazinyl-1-)piperidyl, 4-(N-t-butoxycarbonylpiperazinyl-1-)piperidyl, 4-(N-methylsulfonylpiperazinyl-1-)piperidyl, 4-(N-(2-hydroxyethyl)piperazinyl-1-)piperidyl, 4-(N-(2-cyanoethyl)piperazinyl-1-)piperidyl, 4-(N-(3-hydroxypropyl)piperazinyl-1-)piperidyl, 4-(N-(2-N,N-dimethylaminoethyl)piperazinyl-1-)piperidyl, 4-(N-(2-N,N-diethylethyl)piperazinyl-1 -)piperidyl, 4-(N-(3-N,N-dimethylpropyl)piperazinyl-1-)piperidyl, 4-(N-(3-N,N-diethylpropyl)piperazinyl-1-)piperidyl, 4-(tetrahydropyrrolyl-1-)piperidyl, 4-(3-N,N-dimethylaminotetrahydropyrrolyl-1-)piperidyl, 4-(3-N,N-diethylaminotetrahydropyrrolyl-1-)piperidyl,
      (9) piperidin-4-yl, N-methyl-4-piperidyl, N-ethyl-4-piperidyl, N-isopropyl-4-piperidyl, N-acetyl-4-piperidyl, N-t-butoxycarbonyl-4-piperidyl, N-methylsulfonyl-4-piperidyl, N-(2-hydroxyethyl)-4-piperidyl, N-(2-N,N-dimethylaminoethyl)-4-piperidyl, N-(2-N,N-diethylaminoethyl)-4-piperidyl, N-(2-cyanoethyl)-4-piperidyl, N-(3-hydroxypropyl)-4-piperidyl, N-(3-N,N-dimethylaminopropyl)-4-piperidyl, N-(3-N,N-diethylaminopropyl)-4-piperidyl, N-(3-cyanopropyl)-4-piperidyl, N-cyanomethylene-4-piperidyl,
      (10) amino, N,N-dimethylamino, N,N-diethylamino, N,N-di-n-propylamino, N,N-diisopropylamino, 2-N,N-dimethylaminoethylamino, 2-morpholinylethylamino, 2-(N-methylpiperazinyl)ethylamino, 3-N,N-dimethylaminopropylamino, 3-N,N-diethylaminopropylamino, 3-N,N-diisopropylaminopropylamino, 3-morpholinylpropylamino, 3-(N-methylpiperazinyl)propylamino, N-methylpiperidyl-4-amino, N-ethylpiperidyl-4-amino, N-n-propylpiperidyl-4-amino, N-isopropylpiperidyl-4-amino, acetamido, propionamido, 2-(N,N-dimethylamino)acetamido, 2-hydroxyacetamido, 2-methoxyacetamido, methylsulfamido, ethylsulfamido, n-propylsulfamido, isopropylsulfamido, cyclopropylsulfamido, N-methyl-N-(N-methyl-3-tetrahydropyrrolyl)amino, N-(N-methyl-3-tetrahydropyrrolyl)amino, N-methyl-N-(2-N,N-dimethylamino)ethylamino,
      (11) aminomethylene, N,N-dimethylaminomethylene, N,N-diethylaminomethylene, 4-methyl-1-piperazinylmethylene, 4-ethyl-1-piperazinylmethylene, 4-propyl-1-piperazinylmethylene, 4-isopropyl-1-piperazinylmethylene, 4-(2-hydroxyethyl)-1-piperazinylmethylene, 4-(2-N,N-dimethylaminoethyl)-1-piperazinylmethylene, piperidyl-1-methylene, 4-(N,N-dimethylamino)-1-piperidylmethylene, 4-hydroxypiperidyl-1-methylene, tetrahydropyrrolyl-1-methylene, 3-(N,N-dimethylamino)-1-tetrahydropyrrolylmethylene, benzyl, cyclopropylmethylene, cyclobutylmethylene, cyclopentylmethylene, cyclohexylmethylene,
      (12) aminoformyl, methylaminoformyl, N,N-dimethylaminoformyl, ethylaminoformyl, cyclopropylaminoformyl, cyclobutylaminoformyl, cyclopentylaminoformyl, cyclohexylaminoformyl, aminoacetyl, methylaminoacetyl, 2-(N,N-dimethylamino)acetyl, ethylaminoacetyl, cyclopropylaminoacetyl, cyclobutylaminoacetyl, cyclopentylaminoacetyl, cyclohexylaminoacetyl, tetrahydropyrrolyl-1-formyl, 3-(N,N-dimethylamino)tetrahydropyrrolyl-1-formyl, 3-(N,N-diethylamino)tetrahydropyrrolyl-1-formyl, morpholinyl-4-formyl, thiomorpholinyl-4-formyl, 2,6-dimethylmorpholinyl-4-formyl, piperidyl-1-formyl, 4-hydroxypiperidyl-1-formyl, 4-(N,N-dimethylamino)piperidyl-1-formyl, 4-(N,N-diethylamino)piperidyl-1-formyl, 4-(N-methyl-1-piperazinyl)piperidyl-1-formyl, 4-(N-ethyl-1-piperazinyl)piperidyl-1-formyl, N-methylpiperazinyl-1-formyl, N-ethylpiperazinyl-1-formyl, N-isopropylpiperazinyl-1-formyl, N-(2-hydroxyethyl)piperazinyl-1-formyl, N-(2-N,N-dimethylaminoethyl)piperazinyl-1-formyl, N-(2-N,N-diethylaminoethyl)piperazinyl-1-formyl, 4-(N-methyl-4-piperidyl)piperazinyl-1-formyl, 4-(N-ethyl-4-piperidyl)piperazinyl-1-formyl,
      (13) hydroxyformyl, methoxyformyl, ethoxyformyl, propoxyformyl, isopropoxyformyl, n-butoxyformyl, iso-butoxyformyl, t-butoxyformyl,
      (14) hydroxysulfonyl, aminosulfonyl, methylaminosulfonyl, ethylaminosulfonyl, propylaminosulfonyl, isopropylaminosulfonyl, cyclopropylaminosulfonyl, cyclobutylaminosulfonyl, cyclopentylaminosulfonyl, cyclohexylaminosulfonyl, tetrahydropyrrolyl-1-sulfonyl, 3-(N,N-dimethylamino)tetrahydropyrrolyl-1-sulfonyl, 3-(N,N-diethylamino)tetrahydropyrrolyl-1-sulfonyl, morpholinyl-4-sulfonyl, thiomorpholinyl-4-sulfonyl, 2,6-dimethylmorpholinyl-4-sulfonyl, piperidyl-1-sulfonyl, 4-hydroxypiperidyl-1-sulfonyl, 4-(N,N-dimethylamino)piperidyl-1-sulfonyl, 4-(N,N-diethylamino)piperidyl-1-sulfonyl, 4-(N-methyl-1-piperazinyl)piperidyl-1-sulfonyl, 4-(N-ethyl-1-piperazinyl)piperidyl-1-sulfonyl, N-methylpiperazinyl-1-sulfonyl, N-ethylpiperazinyl-1-sulfonyl, N-isopropylpiperazinyl-1-sulfonyl, N-(2-hydroxyethyl)piperazinyl-1-sulfonyl, N-(2-N,N-dimethylaminoethyl)piperazinyl-1-sulfonyl, N-(2-N,N-diethylaminoethyl)piperazinyl-1-sulfonyl, 4-(N-methyl-4-piperidyl)piperazinyl-1-sulfonyl, 4-(N-ethyl-4-piperidyl)piperazinyl-1-sulfonyl,
      (15) aminoformamido, methylaminoformamido, ethylaminoformamido, propylaminoformamido, isopropylaminoformamido, cyclopropylaminoformamido, cyclobutylaminoformamido, cyclopentylaminoformamido, piperidyl-1-formamido, 4-hydroxypiperidyl-1-formamido, 4-N,N-dimethylpiperidyl-1-formamido, 4-N,N-diethylpiperidyl-1-formamido, tetrahydropyrrolyl-1-formamido, 3-N,N-dimethyltetrahydropyrrolyl-1-formamido, 3-N,N-diethyltetrahydropyrrolyl-1-formamido, N-methylpiperazinyl-1-formamido, N-ethylpiperazinyl-1-formamido, N-acetylpiperazinyl-1-formamido, N-t-butoxycarbonylpiperazinyl-1-formamido, N-(2-hydroxyethyl)piperazinyl-1-formamido, N-(2-cyanoethyl)piperazinyl-1-formamido, N-(2-N,N-dimethylaminoethyl)piperazinyl-1-formamido, N-(2-N,N-diethylaminoethyl)piperazinyl-1-formamido, N-(3-hydroxypropyl)piperazinyl-1-formam ido, N-(3-N,N-dimethylpropyl)piperazinyl-1 -formamido, N-(3-N,N-diethylpropyl)piperazinyl-1-formamido, morpholinyl-1-formamido, 3,5-dimethylmorpholinyl-1-formamido, 4-(tetrahydropyrrolyl)piperidyl-1-formamido, 4-(N-methyl-1-piperazinyl) piperidyl-1-formamido, 4-(N-ethyl-1-piperazinyl)piperidyl-1-formamido, 4-(N-acetyl-1-piperazinyl)piperidyl-1-formamido, N-(N-methyl-4-piperidyl)piperazinyl-1-formamido,
      (16) Z₃ and Z₄ may form an oxygen-containing substituted or unsubstituted 5- to 7-membered ring; the substituent may be selected from the same substituents as described above for Z₁,
      (17) Z₃ and Z₄ may form a nitrogen-containing substituted or unsubstituted 5- to 7-membered ring; the substituent may be selected from the same substituents as described above for Z₁,
   5) 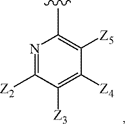
   wherein Z₂, Z₃, Z₄, Z₅ are the same as defined in 4), and Z₂, Z₃, Z₄, Z₅ are not hydrogen at the same time;
   6) 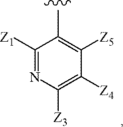
   wherein Z₁, Z₃, Z₄, Z₅ are the same as defined in 4), and Z₁, Z₃, Z₄, Z₅ are not hydrogen at the same time;
   7) 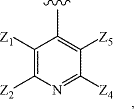
   wherein Z₁, Z₂, Z₄, Z₅ are the same as defined in 4), and Z₁, Z₂, Z₄, Z₅ may be hydrogen at the same time;
   8) 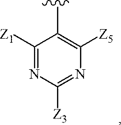
   wherein Z₁, Z₃, Z₅ are the same as defined in 4), and Z₁, Z₃, Z₅ are not hydrogen at the same time;
R², R³ each are independently selected from:
   H, C₁-C₆ alkyl, C₁-C₆ fluorine-containing alkyl, C₃-C₆ cycloalkyl, or C₃-C₆ fluorine-containing cycloalkyl;
R⁴, R⁵ each are independently selected from:
   H, F, Cl, Br, C₁-C₆ fluorine-containing alkyl (e.g., trifluoromethyl), cyano, or nitro, or, R⁴, R⁵ and carbon atoms linked thereto together form a N-, O- or S-containing 5-membered ring.

In some embodiments, R¹ is selected from:
1) H, C₁-C₆ alkyl, C₁-C₆ fluorine-containing alkyl, C₃-C₈ cycloalkyl, C₁-C₆ heteroatom-containing alkyl, C₃-C₈ heteroatom-containing cycloalkyl;
2) 2-N,N-dimethylaminoethyl, 3-N,N-dimethylaminopropyl, 2-N,N-diethylaminoethyl, 3-N,N-diethylaminopropyl, 2-(N-methylpiperazinyl)ethyl, 2-(N-acetylpiperazinyl)ethyl, 2-morpholinylethyl, 2-thiomorpholinylethyl, 2-piperidylethyl, 2-hydroxyethyl, 3-hydroxypropyl, 2-methoxyethyl, N-methyl-4-piperidyl, N-ethyl-4-piperidyl, N-propyl-4-piperidyl, N-isopropyl-4-piperidyl, N-hydroxyethyl-4-piperidyl, N-cyanomethyl-4-piperidyl, N-(2-cyanoethyl)-4-piperidyl, 3-N,N-dimethylaminocyclopentyl, 3-N,N-diethylaminocyclopentyl, 3-N,N-dipropylaminocyclopentyl, 3-N,N-isopropylaminocyclopentyl, 4-aminocyclohexyl, 4-N,N-dimethylaminocyclohexyl, 4-N,N-diethylaminocyclohexyl, 4-N,N-dipropylaminocyclohexyl, 4-N,N-diisopropylaminocyclohexyl, 4-hydroxycyclohexyl, 4-methoxycyclohexyl, 4-ethoxycyclohexyl, 4-isopropoxycyclohexyl;
3) 1-methyl-3-pyrrolyl, 1-ethyl-3-pyrrolyl, 1-isopropyl-3-pyrrolyl, 1-cyclopropyl-3-pyrrolyl, 1-methyl-3-pyrazolyl, 1-ethyl-3-pyrazolyl, 1-isopropyl-3-pyrazolyl, 1-cyclopropyl-3-pyrazolyl, 1-hydroxyethyl-3-pyrazolyl, 1-methyl-4-pyrazolyl, 1-ethyl-4-pyrazolyl, 1-isopropyl-4-pyrazolyl, 1-cyclopropyl-4-pyrazolyl, 1-hydroxyethyl-4-pyrazolyl, 1,3-dimethyl-5-pyrazolyl, 1-methyl-4-imidazolyl, 3-methyl-5-isoxazolyl;
4)

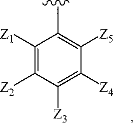

wherein Z₁, Z₂, Z₃, Z₄, Z₅ each are independently selected from the following groups, and Z₁, Z₂, Z₃, Z₄, Z₅ are not hydrogen at the same time:
   (1) H, F, Cl, Br, I, nitro, cyano,
   (2) C₁—C₆ alkyl, C₁—C₆ alkoxy, C₁—C₆ oxygen-containing alkyl, C₁—C₆ fluorine-containing alkyl, C₁—C₆ fluorine-containing alkoxy, C₃—C₆ cycloalkyl, C₂—C₆ alkenyl,
   (3) piperazinyl, N-methylpiperazinyl, N-ethylpiperazinyl, N-n-propylpiperazinyl, N-isopropylpiperazinyl, N-cyclopropylpiperazinyl, N-(2-hydroxyethyl)piperazinyl, N-(2-N,N-dimethylaminoethyl)piperazinyl, N-(2-N,N-diethylaminoethyl)piperazinyl, N-(2-cyanoethyl)piperazinyl, N-cyanomethylpiperazinyl, N-(3-hydroxypropyl)piperazinyl, N-(3-N,N-dimethylaminopropyl)piperazinyl, N-(3-N,N-diethylaminopropyl)piperazinyl,
   (4) morpholinyl, 2,6-dimethylmorpholinyl, thiomorpholinyl,
   (5) tetrahydropyrrolyl, 3-(N,N-dimethylamino)tetrahydropyrrolyl, 3-(N,N-diethylamino)tetrahydropyrrolyl,
   (6) imidazolyl, 4-methyl-1-imidazolyl,
   (7) piperidyl, 4-N,N-dimethylaminopiperidyl, 4-N,N-diethylaminopiperidyl, 4-N,N-diisopropylaminopiperidyl, 4-hydroxypiperidyl, 4-methoxypiperidyl, 4-ethoxypiperidyl, 4-(N-methylpiperazinyl-1-)piperidyl, 4-(N-ethylpiperazinyl-1-)piperidyl, 4-(N-isopropylpiperazinyl-1-)piperidyl, 4-(N-(2-hydroxyethyl)piperazinyl-1-)piperidyl, 4-(N-(2-cyanoethyl)piperazinyl-1-)piperidyl, 4-(N-(3-hydroxypropyl)piperazinyl-1-)piperidyl, 4-(N-(2-N,N-dimethylaminoethyl)piperazinyl-1-)piperidyl, 4-(N-(2-N,N-diethylethyl)piperazinyl-1-)piperidyl, 4-(N-(3-N,N-dimethylpropyl)piperazinyl-1-)piperidyl, 4-(N-(3-N,N-diethylpropyl)piperazinyl-1-)piperidyl, 4-(tetrahydropyrrolyl-1-)piperidyl, 4-(3-N,N-dimethylaminotetrahydropyrrolyl-1-)piperidyl, 4-(3-N,N-diethylaminotetrahydropyrrolyl-1-)piperidyl,
   (8) piperidin-4-yl, N-methyl-4-piperidyl, N-ethyl-4-piperidyl, N-isopropyl-4-piperidyl, N-(2-hydroxyethyl)-4-piperidyl, N-(2-N,N-dimethylaminoethyl)-4-piperidyl, N-(2-N,N-diethylaminoethyl)-4-piperidyl, N-(2-cyanoethyl)-4-piperidyl, N-(3-hydroxypropyl)-4-piperidyl, N-(3-N,N-dimethylaminopropyl)-4-piperidyl, N-(3-N,N-diethylaminopropyl)-4-piperidyl, N-(3-cyanopropyl)-4-piperidyl, N-cyanomethylene-4-piperidyl,
   (9) amino, N,N-dimethylamino, N,N-diethylamino, N,N-di-n-propylamino, N,N-diisopropylamino, 2-N,N-dimethylaminoethylamino, 3-N,N-dimethylaminopropylamino, 3-N,N-diethylaminopropylamino, 3-N,N-diisopropylaminopropylamino, N-methylpiperidyl-4-amino, N-ethylpiperidyl-4-amino, N-n-propylpiperidyl-4-amino, N-isopropylpiperidyl-4-amino, acetamido, propionamido, 2-(N,N-dimethylamino)acetamido, 2-hydroxyacetamido, 2-methoxyacetamido, methylsulfamido, ethylsulfamido, n-propylsulfamido, isopropylsulfamido, cyclopropylsulfamido, N-methyl-N-(N-methyl-3-tetrahydropyrrolyl)amino, N-(N-methyl-3-tetrahydropyrrolyl)amino, N-methyl-N-(2-N,N-dimethylamino)ethylamino,
   (10) 4-methyl-1-piperazinylmethylene, 4-ethyl-1-piperazinylmethylene, 4-propyl-1-piperazinylmethylene, 4-isopropyl-1-piperazinylmethylene, 4-(2-hydroxyethyl)-1-piperazinylmethylene, 4-(2-N,N-dimethylaminoethyl)-1-piperazinylmethylene, piperidyl-1-methylene, 4-(N,N-dimethylamino)-1-piperidylmethylene, 4-hydroxypiperidyl-1-methylene, tetrahydropyrrolyl-1-methylene, 3-(N,N-dimethylamino)-1-tetrahydropyrrolylmethylene, benzyl, cyclopropylmethylene, cyclobutylmethylene, cyclopentylmethylene, cyclohexylmethylene,
   (11) aminoformyl, methylaminoformyl, N,N-dimethylaminoformyl, ethylaminoformyl, aminoacetyl, methylaminoacetyl, 2-(N,N-dimethylamino)acetyl, ethylaminoacetyl, tetrahydropyrrolyl-1-formyl, 3-(N,N-dimethylamino)tetrahydropyrrolyl-1-formyl, 3-(N,N-diethylamino)tetrahydropyrrolyl-1-formyl, morpholinyl-4-formyl, thiomorpholinyl-4-formyl, 2,6-dimethylmorpholinyl-4-formyl, piperidyl-1-formyl, 4-hydroxypiperidyl-1-formyl, 4-(N,N-dimethylamino)piperidyl-1-formyl, 4-(N,N-diethylamino)piperidyl-1-formyl, 4-(N-methyl-1-piperazinyl)piperidyl-1-formyl, 4-(N-ethyl-1-piperazinyl)piperidyl-1-formyl, N-methylpiperazinyl-1-formyl, N-ethylpiperazinyl-1-formyl, N-isopropylpiperazinyl-1-formyl, N-(2-hydroxyethyl)piperazinyl-1-formyl, N-(2-N,N-dimethylaminoethyl)piperazinyl-1-formyl, N-(2-N,N-diethylaminoethyl)piperazinyl-1-formyl, 4-(N-methyl-4-piperidyl)piperazinyl-1-formyl, 4-(N-ethyl-4-piperidyl)piperazinyl-1-formyl,
   (12) hydroxysulfonyl, aminosulfonyl, methylaminosulfonyl, ethylaminosulfonyl, propylaminosulfonyl, isopropylaminosulfonyl, cyclopropylaminosulfonyl, cyclobutylaminosulfonyl, cyclopentylaminosulfonyl, cyclohexylaminosulfonyl, tetrahydropyrrolyl-1-sulfonyl, 3-(N,N-dimethylamino)tetrahydropyrrolyl-1-sulfonyl, 3-(N,N-diethylamino)tetrahydropyrrolyl-1-sulfonyl, morpholinyl-4-sulfonyl, thiomorpholinyl-4-sulfonyl, 2,6-dimethylmorpholinyl-4-sulfonyl, piperidyl-1-sulfonyl, 4-hydroxypiperidyl-1-sulfonyl, 4-(N,N-dimethylamino)piperidyl-1-sulfonyl, 4-(N,N-diethylamino)piperidyl-1-sulfonyl, N-methylpiperazinyl-1-sulfonyl, N-ethylpiperazinyl-1-sulfonyl, N-isopropylpiperazinyl-1-sulfonyl, N-(2-hydroxyethyl)piperazinyl-1-sulfonyl, N-(2-N,N-dimethylaminoethyl)piperazinyl-1-sulfonyl, N-(2-N,N-diethylaminoethyl)piperazinyl-1-sulfonyl,
   (13) aminoformamido, methylaminoformamido, ethylaminoformamido, propylaminoformamido, isopropylaminoformamido;
5)

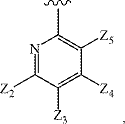

wherein Z₂, Z₃, Z₄, Z₅ are the same as defined in 4), and Z₂, Z₃, Z₄, Z₅ are not hydrogen at the same time;
6)

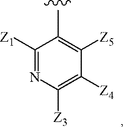

wherein Z₁, Z₃, Z₄, Z₅ are the same as defined in 4), and Z₁, Z₃, Z₄, Z₅ are not hydrogen at the same time;
7)

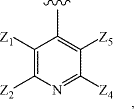

wherein Z₁, Z₂, Z₄, Z₅ are the same as defined in 4), and Z₁, Z₂, Z₄, Z₅ may be hydrogen at the same time;
8)

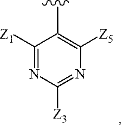

wherein Z₁, Z₃, Z₅ are the same as defined in 4), and Z₁, Z₃, Z₅ are not hydrogen at the same time;
9)

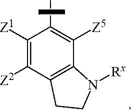

wherein Z₁, Z₂, Z₅ are the same as defined in 4), and Z₁, Z₂, Z₅ may be hydrogen at the same time, R^{x} is selected from the same substituents as described above for Z¹;
10)

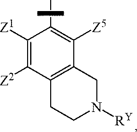

wherein Z₁, Z₂, Z₅ are the same as defined in 4), and Z₁, Z₂, Z₅ may be hydrogen at the same time, R^{y} is selected from the same substituents as described above for Z¹;
11)

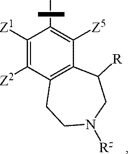

wherein Z₁, Z₂, Z₅ are the same as defined in 4), and Z₁, Z₂, Z₅ may be hydrogen at the same time, R^{z}, R each are independently selected from the same substituents as described above for Z¹.

In some embodiments, R¹ is selected from:
1) H, C₁-C₆ alkyl, C₃-C₈ cycloalkyl;
2) 2-N,N-dimethylaminoethyl, 3-N,N-dimethylaminopropyl, 2-N,N-diethylaminoethyl, 3-N,N-diethylaminopropyl, 2-hydroxyethyl, 3-hydroxypropyl, N-methyl-4-piperidyl, N-ethyl-4-piperidyl, N-propyl-4-piperidyl, N-isopropyl-4-piperidyl, 4-aminocyclohexyl, 4-N,N-dimethylaminocyclohexyl, 4-N,N-diethylaminocyclohexyl, 4-N,N-dipropylaminocyclohexyl, 4-N,N-diisopropylaminocyclohexyl;
3) 1-methyl-3-pyrazolyl, 1-ethyl-3-pyrazolyl, 1-isopropyl-3-pyrazolyl, 1-cyclopropyl-3-pyrazolyl, 1-methyl-4-pyrazolyl, 1-ethyl-4-pyrazolyl, 1-isopropyl-4-pyrazolyl, 1-cyclopropyl-4-pyrazolyl, 1,3-dimethyl-5-pyrazolyl, 1-methyl-4-imidazolyl;
4)

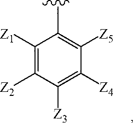

wherein Z₁, Z₂, Z₃, Z₄, Z₅ each are independently selected from the following groups, and Z₁, Z₂, Z₃, Z₄, Z₅ are not hydrogen at the same time:
   (1) H, F, Cl, Br, I, nitro, cyano,
   (2) C₁-C₆ alkyl, C₁-C₆ alkoxy, C₁-C₆ fluorine-containing alkyl, C₁-C₆ fluorine-containing alkoxy, C₂-C₆ alkenyl,
   (3) piperazinyl, N-methylpiperazinyl, N-ethylpiperazinyl, N-n-propylpiperazinyl, N-isopropylpiperazinyl,
   (4) morpholinyl, 2,6-dimethylmorpholinyl, thiomorpholinyl,
   (5) 3-(N,N-dimethylamino)tetrahydropyrrolyl, 3-(N,N-diethylamino)tetrahydropyrrolyl,
   (6) 4-N,N-dimethylaminopiperidyl, 4-N,N-diethylaminopiperidyl, 4-N,N-diisopropylaminopiperidyl, 4-hydroxypiperidyl, 4-(N-methylpiperazinyl-1-)piperidyl, 4-(N-ethylpiperazinyl-1-)piperidyl, 4-(N-isopropylpiperazinyl-1-)piperidyl, 4-(tetrahydropyrrolyl-1-)piperidyl,
   (7) piperidin-4-yl, N-methyl-4-piperidyl, N-ethyl-4-piperidyl, N-isopropyl-4-piperidyl,
   (8) aminoformamido, methylaminoformamido, ethylaminoformamido, propylaminoformamido, isopropylaminoformamido, 2-(N,N-dimethylamino)acetamido, cyclopropylsulfamido, 2-N,N-dimethylaminoethylamino, 3-N,N-dimethylaminopropylamino, 3-N,N-diethylaminopropylamino, 3-N,N-diisopropylaminopropylamino, N-methyl-N-(2-N,N-dimethylamino)ethylamino,
   (9) 4-methyl-1-piperazinylmethylene, 4-ethyl-1-piperazinylmethylene, 4-propyl-1-piperazinylmethylene, 4-isopropyl-1-piperazinylmethylene, benzyl, cyclopropylmethylene, cyclobutylmethylene, cyclopentylmethylene, cyclohexylmethylene,
   (10) 4-(N-methyl-1-piperazinyl)piperidyl-1-formyl, 4-(N-ethyl-1-piperazinyl)piperidyl-1-formyl, N-methylpiperazinyl-1-formyl, N-ethylpiperazinyl-1-formyl, N-isopropylpiperazinyl-1-formyl,
   (11) cyclopropylaminosulfonyl, cyclobutylaminosulfonyl, cyclopentylaminosulfonyl, cyclohexylaminosulfonyl, N-methylpiperazinyl-1-sulfonyl, N-ethylpiperazinyl-1-sulfonyl, N-isopropylpiperazinyl-1-sulfonyl;
5)

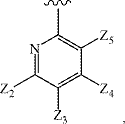

wherein Z₂, Z₃, Z₄, Z₅ each are independently selected from H, C₁-C₆ alkyl, C₁-C₆ alkoxy, piperazinyl, N-methylpiperazinyl, N-ethylpiperazinyl, N-n-propylpiperazinyl, or N-isopropylpiperazinyl, and Z₂, Z₃, Z₄, Z₅ are not hydrogen at the same time;
6)

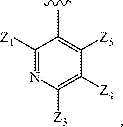

wherein Z₁, Z₃, Z₄, Z₅ each are independently selected from H, C₁—C₆ alkyl, C₁—C₆ alkoxy, N-(N-methyl-3-tetrahydropyrrolyl)amino, N-methyl-N-(N-methyl-3-tetrahydropyrrolyl)amino, N-methylpiperazinyl, N-ethylpiperazinyl, N-n-propylpiperazinyl, N-isopropylpiperazinyl, 4-(N-methylpiperazinyl-1-)piperidyl, 4-(N-ethylpiperazinyl-1-)piperidyl, or 4-(N-isopropylpiperazinyl-1-)piperidyl, and Z₁, Z₃, Z₄, Z₅ are not hydrogen at the same time;
7)

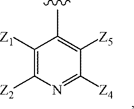

wherein Z₁, Z₂, Z₄, Z₅ each are independently selected from H, or C₁-C₆ alkyl, and Z₁, Z₂, Z₄, Z₅ may be hydrogen at the same time;
8)

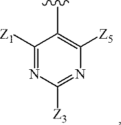

wherein Z₁, Z₃, Z₅ each are independently selected from H, C₁—C₆ alkyl, C₁—C₆ alkoxy, N-methylpiperazinyl, N-ethylpiperazinyl, N-n-propylpiperazinyl, or N-isopropylpiperazinyl, and Z₁, Z₃, Z₅ are not hydrogen at the same time;
9)

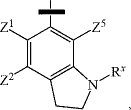

wherein Z₁, Z₂, Z₅ each are independently selected from H, C₁—C₆ alkyl, or C₁—C₆ alkoxy, and Z₁, Z₂, Z₅ may be hydrogen at the same time, R^{x} is selected from aminoformyl, methylaminoformyl, N,N-dimethylaminoformyl, ethylaminoformyl, aminoacetyl, methylaminoacetyl, 2-(N,N-dimethylamino)acetyl, or ethylaminoacetyl;
10)

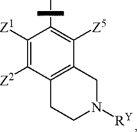

wherein Z₁, Z₂, Z₅ each are independently selected from H, F, Cl, Br, I, C₁—C₆ alkyl, C₁—C₆ alkoxy, C₁—C₆ fluorine-containing alkyl, or C₁—C₆ fluorine-containing alkoxy, and Z₁, Z₂, Z₅ may be hydrogen at the same time, R^{y} is C₁—C₆ alkyl;
11)

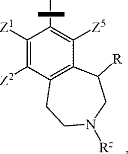

wherein Z₁, Z₂, Z₅ each are independently selected from H, or C₁—C₆ alkyl, and Z₁, Z₂, Z₅ may be hydrogen at the same time, R^{z}, R each are independently selected from C₁—C₆ alkyl.

In some embodiments, R¹ is selected from:
1) cyclopropyl, 2-N,N-dimethylaminoethyl, 2-hydroxyethyl, N-methyl-4-piperidyl, 4-aminocyclohexyl, 1-methyl-3-pyrazolyl, 1-methyl-4-pyrazolyl, 1,3-dimethyl-5-pyrazolyl, 1-methyl-4-imidazolyl;
2)

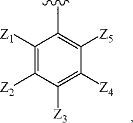

wherein Z₁, Z₂, Z₃, Z₄, Z₅ each are independently selected from the following groups, and Z₁, Z₂, Z₃, Z₄, Z₅ are not hydrogen at the same time: H, methyl, ethyl, methoxy, ethoxy, isopropoxy, trifluoromethyl, trifluoromethoxy, vinyl, N-methylpiperazinyl, morpholinyl, 3-(N,N-dimethylamino)tetrahydropyrrolyl, 4-N,N-dimethylaminopiperidyl, 4-hydroxypiperidyl, 4-(N-methylpiperazinyl-1-)piperidyl, 4-(tetrahydropyrrolyl-1-)piperidyl, N-methyl-4-piperidyl, 2-(N,N-dimethylamino)acetamido, cyclopropylsulfamido, N-methyl-N-(2-N,N-dimethylamino)ethylamino, 4-methyl-1-piperazinylmethylene, benzyl, cyclopropylmethylene, 4-(N-methyl-1-piperazinyl)piperidyl-1-formyl, N-methylpiperazinyl-1-formyl, cyclopropylaminosulfonyl, or N-methylpiperazinyl-1-sulfonyl;
3)

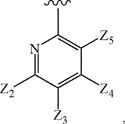

wherein Z₂, Z₃, Z₄, Z₅ each are independently selected from H, methoxy, or N-methylpiperazinyl, and Z₂, Z₃, Z₄, Z₅ are not hydrogen at the same time;
4)

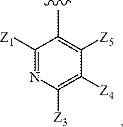

wherein Z₁, Z₃, Z₄, Z₅ each are independently selected from H, methyl, ethyl, methoxy, N-(N-methyl-3-tetrahydropyrrolyl)amino, N-methyl-N-(N-methyl-3-tetrahydropyrrolyl)amino, N-methylpiperazinyl, or 4-(N-methylpiperazinyl-1-)piperidyl, and Z₁, Z₃, Z₄, Z₅ are not hydrogen at the same time;
5)

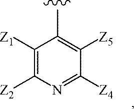

wherein Z₁, Z₂, Z₄, Z₅ are H;
6)

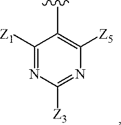

wherein Z₁, Z₃, Z₅ each are independently selected from H, methoxy, or N-methylpiperazinyl, and Z₁, Z₃, Z₅ are not hydrogen at the same time;
7)

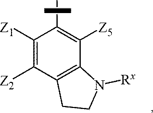

wherein Z₁, Z₂, Z₅ each are independently selected from H, or methoxy, R^{x} is 2-(N,N-dimethylamino)acetyl;
8)

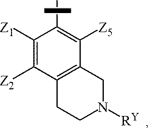

wherein Z₁, Z₂, Z₅ each are independently selected from H, F, Cl, methyl, methoxy, trifluoromethyl, or trifluoromethoxy, R^{y} is methyl;
9)

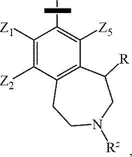

wherein Z₁, Z₂, Z₅ are H, R^{z}, R are methyl.

In some embodiments, R², R³ each are independently selected from methyl, trifluoromethyl, ethyl, isopropyl, or cyclopropyl.

In some embodiments, R⁴ is H, R⁵ is selected from F, Cl, Br, trifluoromethyl, cyano or nitro or, R⁴, R⁵ and carbon atoms linked thereto together form

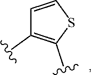

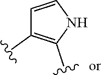

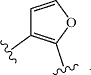

In the eighth aspect of the present invention, the present invention provides a compound having the general formula (I):

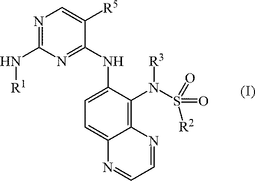

or a stereoisomer, a prodrug, a pharmaceutically acceptable salt or a pharmaceutically acceptable solvate thereof, wherein:
R¹ is selected from:
   1) H, C₁-C₆ alkyl, C₁-C₆ fluorine-containing alkyl, C₃-C₈ cycloalkyl, C₁-C₆ heteroatom-containing alkyl, C₃-C₈ heteroatom-containing cycloalkyl;
   2) 2-N,N-dimethylaminoethyl, 3-N,N-dimethylaminopropyl, 2-N,N-diethylaminoethyl, 3-N,N-diethylaminopropyl, 2-(N-methylpiperazinyl)ethyl, 2-(N-acetylpiperazinyl)ethyl, 2-morpholinylethyl, 2-thiomorpholinylethyl, 2-piperidylethyl, 2-hydroxyethyl, 3-hydroxypropyl, 2-methoxyethyl, N-methyl-4-piperidyl, N-ethyl-4-piperidyl, N-propyl-4-piperidyl, N-isopropyl-4-piperidyl, N-hydroxyethyl-4-piperidyl, N-cyanomethyl-4-piperidyl, N-(2-cyanoethyl)-4-piperidyl, N-acetyl-4-piperidyl, N-propionyl-4-piperidyl, N-isopropionyl-4-piperidyl, N-cyclopropylformyl-4-piperidyl, N-methylsulfonyl-4-piperidyl, N-ethylsulfonyl-4-piperidyl, N-propylsulfonyl-4-piperidyl, N-isopropylsulfonyl-4-piperidyl, N-cyclopropylsulfonyl-4-piperidyl, 3-N,N-dimethylaminocyclopentyl, 3-N,N-diethylaminocyclopentyl, 3-N,N-dipropylaminocyclopentyl, 3-N,N-isopropylaminocyclopentyl, 4-aminocyclohexyl, 4-N,N-dimethylaminocyclohexyl, 4-N,N-diethylaminocyclohexyl, 4-N,N-dipropylaminocyclohexyl, 4-N,N-diisopropylaminocyclohexyl, 4-acetylaminocyclohexyl, 4-propionylaminocyclohexyl, 4-isopropylformylaminocyclohexyl, 4-methylsulfonylaminocyclohexyl, 4-ethylsulfonylaminocyclohexyl, 4-propylsulfonylaminocyclohexyl, 4-isopropylsulfonylaminocyclohexyl, 4-cyclopropylsulfonylaminocyclohexyl, 4-hydroxycyclohexyl, 4-methoxycyclohexyl, 4-ethoxycyclohexyl, 4-isopropoxycyclohexyl;
   3) 1-methyl-3-pyrrolyl, 1-ethyl-3-pyrrolyl, 1-isopropyl-3-pyrrolyl, 1-cyclopropyl-3-pyrrolyl, 1-methyl-3-pyrazolyl, 1-ethyl-3-pyrazolyl, 1-isopropyl-3-pyrazolyl, 1-cyclopropyl-3-pyrazolyl, 1-hydroxyethyl-3-pyrazolyl, 1-methyl-4-pyrazolyl, 1-ethyl-4-pyrazolyl, 1-isopropyl-4-pyrazolyl, 1-cyclopropyl-4-pyrazolyl, 1-hydroxyethyl-4-pyrazolyl, 1-(N-methyl-4-piperidyl)-4-pyrazolyl, 1-(N-t-butoxycarbonyl-4-piperidyl)-4-pyrazolyl, 1,3-dimethyl-5-pyrazolyl, 1-methyl-4-imidazolyl, 3-methyl-5-isoxazolyl;
   4) 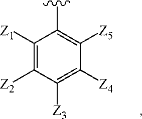
   wherein Z₁, Z₂, Z₃, Z₄, Z₅ each are independently selected from the following groups, and Z₁, Z₂, Z₃, Z₄, Z₅ are not hydrogen at the same time:
      (1) H, F, Cl, Br, I, nitro, cyano,
      (2) C₁—C₆ alkyl, C₁—C₆ alkoxy, C₁—C₆ oxygen-containing alkyl, C₁—C₆ fluorine-containing alkyl, C₁—C₆ fluorine-containing alkoxy, C₃—C₆ cycloalkyl, C₂—C₆ alkenyl,
      (3) 2-N,N-dimethylaminoethoxy, 3-N,N-dimethylaminopropoxy, 2-(N-methylpiperazinyl)ethoxy, 2-(N-acetylpiperazinyl)ethoxy, 2-morpholinylethoxy, 2-thiomorpholinylethoxy, 2-piperidylethoxy, 3-N,N-dimethylaminopropoxy, 3-N,N-diethylaminopropoxy, 3-N,N-diisopropylaminopropoxy, 3-(N-methylpiperazinyl)propoxy, 3-(N-acetylpiperazinyl)propoxy, 3-morpholinylpropoxy, 3-thiomorpholinylpropoxy, 3-piperidylpropoxy, 2-pyridylmethoxy, 3-pyridylmethoxy, 4-pyridylmethoxy, phenylmethoxy, monohalo-substituted phenylmethoxy, gem-dihalo-substituted phenylmethoxy, hetero-dihalo-substituted phenylmethoxy,
      (4) piperazinyl, N-methylpiperazinyl, N-ethylpiperazinyl, N-n-propylpiperazinyl, N-isopropylpiperazinyl, N-cyclopropylpiperazinyl, N-(2-hydroxyethyl)piperazinyl, N-(2-N,N-dimethylaminoethyl)piperazinyl, N-(2-N,N-diethylaminoethyl)piperazinyl, N-(2-cyanoethyl)piperazinyl, N-cyanomethylpiperazinyl, N-(3-hydroxypropyl)piperazinyl, N-(3-N,N-dimethylaminopropyl)piperazinyl, N-(3-N,N-diethylaminopropyl)piperazinyl, N-(N-methyl-4-piperidyl)piperazinyl, N-(N-ethyl-4-piperidyl)piperazinyl, N-t-butoxycarbonylpiperazinyl, N-acetylpiperazinyl, N-propionylpiperazinyl, N-isobutyrylpiperazinyl, N-cyclopropylformylpiperazinyl, N-methylsulfonylpiperazinyl, N-ethylsulfonylpiperazinyl, N-n-propylsulfonylpiperazinyl, N-isopropylsulfonylpiperazinyl, N-cyclopropylsulfonylpiperazinyl, 2-oxo-piperazin-4-yl,
      (5) morpholinyl, 2,6-dimethylmorpholinyl, thiomorpholinyl,
      (6) tetrahydropyrrolyl, 3-(N, N-dimethylamino)tetrahydropyrrolyl, 3-(N,N-diethylamino)tetrahydropyrrolyl,
      (7) imidazolyl, 4-methyl-1-imidazolyl,
      (8) piperidyl, 4-N,N-dimethylaminopiperidyl, 4-N,N-diethylaminopiperidyl, 4-N,N-diisopropylaminopiperidyl, 4-hydroxypiperidyl, 4-methoxypiperidyl, 4-ethoxypiperidyl, 4-(N-methylpiperazinyl-1-)piperidyl, 4-(N-ethylpiperazinyl-1-)piperidyl, 4-(N-isopropylpiperazinyl-1-)piperidyl, 4-(N-acetylpiperazinyl-1-)piperidyl, 4-(N-t-butoxycarbonylpiperazinyl-1-)piperidyl, 4-(N-methylsulfonylpiperazinyl-1-)piperidyl, 4-(N-(2-hydroxyethyl)piperazinyl-1-)piperidyl, 4-(N-(2-cyanoethyl)piperazinyl-1-)piperidyl, 4-(N-(3-hydroxypropyl)piperazinyl-1-)piperidyl, 4-(N-(2-N,N-dimethylaminoethyl)piperazinyl-1-)piperidyl, 4-(N-(2-N,N-diethylethyl)piperazinyl-1 -)piperidyl, 4-(N-(3-N,N-dimethylpropyl)piperazinyl-1-)piperidyl, 4-(N-(3-N,N-diethylpropyl)piperazinyl-1-)piperidyl, 4-(tetrahydropyrrolyl-1-)piperidyl, 4-(3-N,N-dimethylaminotetrahydropyrrolyl-1-)piperidyl, 4-(3-N,N-diethylaminotetrahydropyrrolyl-1-)piperidyl,
      (9) piperidin-4-yl, N-methyl-4-piperidyl, N-ethyl-4-piperidyl, N-isopropyl-4-piperidyl, N-acetyl-4-piperidyl, N-t-butoxycarbonyl-4-piperidyl, N-methylsulfonyl-4-piperidyl, N-(2-hydroxyethyl)-4-piperidyl, N-(2-N,N-dimethylaminoethyl)-4-piperidyl, N-(2-N,N-diethylaminoethyl)-4-piperidyl, N-(2-cyanoethyl)-4-piperidyl, N-(3-hydroxypropyl)-4-piperidyl, N-(3-N,N-dimethylaminopropyl)-4-piperidyl, N-(3-N,N-diethylaminopropyl)-4-piperidyl, N-(3-cyanopropyl)-4-piperidyl, N-cyanomethylene-4-piperidyl,
      (10) amino, N,N-dimethylamino, N,N-diethylamino, N,N-di-n-propylamino, N,N-diisopropylamino, 2-N,N-dimethylaminoethylamino, 2-morpholinylethylamino, 2-(N-methylpiperazinyl)ethylamino, 3-N,N-dimethylaminopropylamino, 3-N,N-diethylaminopropylamino, 3-N,N-diisopropylaminopropylamino, 3-morpholinylpropylamino, 3-(N-methylpiperazinyl)propylamino, N-methylpiperidyl-4-amino, N-ethylpiperidyl-4-amino, N-n-propylpiperidyl-4-amino, N-isopropylpiperidyl-4-amino, acetamido, propionamido, 2-(N,N-dimethylamino)acetamido, 2-hydroxyacetamido, 2-methoxyacetamido, methylsulfamido, ethylsulfamido, n-propylsulfamido, isopropylsulfamido, cyclopropylsulfamido, N-methyl-N-(N-methyl-3-tetrahydropyrrolyl)amino, N-(N-methyl-3-tetrahydropyrrolyl)amino, N-methyl-N-(2-N,N-dimethylamino)ethylamino,
      (11) aminomethylene, N,N-dimethylaminomethylene, N,N-diethylaminomethylene, 4-methyl-1-piperazinylmethylene, 4-ethyl-1-piperazinylmethylene, 4-propyl-1-piperazinylmethylene, 4-isopropyl-1-piperazinylmethylene, 4-(2-hydroxyethyl)-1-piperazinylmethylene, 4-(2-N,N-dimethylaminoethyl)-1-piperazinylmethylene, piperidyl-1-methylene, 4-(N,N-dimethylamino)-1-piperidylmethylene, 4-hydroxypiperidyl-1-methylene, tetrahydropyrrolyl-1-methylene, 3-(N,N-dimethylamino)-1-tetrahydropyrrolylmethylene, benzyl, cyclopropylmethylene, cyclobutylmethylene, cyclopentylmethylene, cyclohexylmethylene,
      (12) aminoformyl, methylaminoformyl, N,N-dimethylaminoformyl, ethylaminoformyl, cyclopropylaminoformyl, cyclobutylaminoformyl, cyclopentylaminoformyl, cyclohexylaminoformyl, aminoacetyl, methylaminoacetyl, 2-(N,N-dimethylamino)acetyl, ethylaminoacetyl, cyclopropylaminoacetyl, cyclobutylaminoacetyl, cyclopentylaminoacetyl, cyclohexylaminoacetyl, tetrahydropyrrolyl-1-formyl, 3-(N, N-dimethylamino)tetrahydropyrrolyl-1-formyl, 3-(N,N-diethylamino)tetrahydropyrrolyl-1-formyl, morpholinyl-4-formyl, thiomorpholinyl-4-formyl, 2,6-dimethylmorpholinyl-4-formyl, piperidyl-1-formyl, 4-hydroxypiperidyl-1-formyl, 4-(N,N-dimethylamino)piperidyl-1-formyl, 4-(N,N-diethylamino)piperidyl-1-formyl, 4-(N-methyl-1-piperazinyl)piperidyl-1-formyl, 4-(N-ethyl-1-piperazinyl)piperidyl-1-formyl, N-methylpiperazinyl-1-formyl, N-ethylpiperazinyl-1-formyl, N-isopropylpiperazinyl-1-formyl, N-(2-hydroxyethyl)piperazinyl-1-formyl, N-(2-N,N-dimethylaminoethyl)piperazinyl-1-formyl, N-(2-N,N-diethylaminoethyl)piperazinyl-1-formyl, 4-(N-methyl-4-piperidyl)piperazinyl-1-formyl, 4-(N-ethyl-4-piperidyl)piperazinyl-1-formyl,
      (13) hydroxyformyl, methoxyformyl, ethoxyformyl, propoxyformyl, isopropoxyformyl, n-butoxyformyl, iso-butoxyformyl, t-butoxyformyl,
      (14)hydroxysulfonyl, aminosulfonyl, methylaminosulfonyl, ethylaminosulfonyl, propylaminosulfonyl, isopropylaminosulfonyl, cyclopropylaminosulfonyl, cyclobutylaminosulfonyl, cyclopentylaminosulfonyl, cyclohexylaminosulfonyl, tetrahydropyrrolyl-1-sulfonyl, 3-(N,N-dimethylamino)tetrahydropyrrolyl-1-sulfonyl, 3-(N,N-diethylamino)tetrahydropyrrolyl-1-sulfonyl, morpholinyl-4-sulfonyl, thiomorpholinyl-4-sulfonyl, 2,6-dimethylmorpholinyl-4-sulfonyl, piperidyl-1-sulfonyl, 4-hydroxypiperidyl-1-sulfonyl, 4-(N,N-dimethylamino)piperidyl-1-sulfonyl, 4-(N,N-diethylamino)piperidyl-1-sulfonyl, 4-(N-methyl-1-piperazinyl)piperidyl-1-sulfonyl, 4-(N-ethyl-1-piperazinyl)piperidyl-1-sulfonyl, N-methylpiperazinyl-1-sulfonyl, N-ethylpiperazinyl-1-sulfonyl, N-isopropylpiperazinyl-1-sulfonyl, N-(2-hydroxyethyl)piperazinyl-1-sulfonyl, N-(2-N,N-dimethylaminoethyl)piperazinyl-1-sulfonyl, N-(2-N,N-diethylaminoethyl)piperazinyl-1-sulfonyl, 4-(N-methyl-4-piperidyl )piperazinyl-1-sulfonyl, 4-(N-ethyl-4-piperidyl)piperazinyl-1-sulfonyl,
      (15) aminoformamido, methylaminoformamido, ethylaminoformamido, propylaminoformamido, isopropylaminoformamido, cyclopropylaminoformamido, cyclobutylaminoformamido, cyclopentylaminoformamido, piperidyl-1-formamido, 4-hydroxypiperidyl-1-formamido, 4-N,N-dimethylpiperidyl-1-formamido, 4-N,N-diethylpiperidyl-1-formamido, tetrahydropyrrolyl-1-formamido, 3-N,N-dimethyltetrahydropyrrolyl-1-formamido, 3-N,N-diethyltetrahydropyrrolyl-1-formamido, N-methylpiperazinyl-1-formamido, N-ethylpiperazinyl-1-formamido, N-acetylpiperazinyl-1-formamido, N-t-butoxycarbonylpiperazinyl-1-formamido, N-(2-hydroxyethyl)piperazinyl-1-formamido, N-(2-cyanoethyl)piperazinyl-1-formam ido, N-(2-N,N-dimethylaminoethyl)piperazinyl-1-formam ido, N-(2-N,N-diethylaminoethyl)piperazinyl-1-formamido, N-(3-hydroxypropyl)piperazinyl-1-formamido, N-(3-N,N-dimethylpropyl)piperazinyl-1 -formamido, N-(3-N,N-diethylpropyl)piperazinyl-1-formamido, morpholinyl-1-formamido, 3,5-dimethylmorpholinyl-1-formamido, 4-(tetrahydropyrrolyl)piperidyl-1-formamido, 4-(N-methyl-1-piperazinyl)piperidyl-1-formamido, 4-(N-ethyl-1-piperazinyl)piperidyl-1-formamido, 4-(N-acetyl-1-piperazinyl)piperidyl-1-formamido, N-(N-methyl-4-piperidyl)piperazinyl-1-formamido,
      (16) Z₃ and Z₄ may form an oxygen-containing substituted or unsubstituted 5- to 7-membered ring; the substituent may be selected from the same substituents as described above for Z₁,
      (17) Z₃ and Z₄ may form a nitrogen-containing substituted or unsubstituted 5- to 7-membered ring; the substituent may be selected from the same substituents as described above for Z₁,
   5) 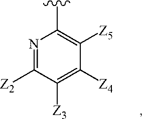
   wherein Z₂, Z₃, Z₄, Z₅ are the same as defined in 4), and Z₂, Z₃, Z₄, Z₅ are not hydrogen at the same time;
   6) 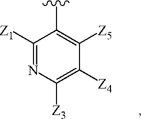
   wherein Z₁, Z₃, Z₄, Z₅ are the same as defined in 4), and Z₁, Z₃, Z₄, Z₅ are not hydrogen at the same time;
   7) 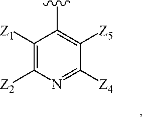
   wherein Z₁, Z₂, Z₄, Z₅ are the same as defined in 4), and Z₁, Z₂, Z₄, Z₅ may be hydrogen at the same time;
   8) 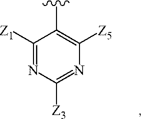
   wherein Z₁, Z₃, Z₅ are the same as defined in 4), and Z₁, Z₃, Z₅ are not hydrogen at the same time;
R², R³ each are independently selected from H, C₁-C₆ alkyl, C₁-C₆ fluorine-containing alkyl, C₃-C₆ cycloalkyl, or C₃-C₆ fluorine-containing cycloalkyl;
R⁵ is selected from F, Cl, Br, C₁-C₆ fluorine-containing alkyl, cyano, or nitro.

In some embodiments, R¹ is selected from:
1) H, C₁-C₆ alkyl, C₁-C₆ fluorine-containing alkyl, C₃-C₈ cycloalkyl, C₁-C₆ heteroatom-containing alkyl, C₃-C₈ heteroatom-containing cycloalkyl;
2) 2-N,N-dimethylaminoethyl, 3-N,N-dimethylaminopropyl, 2-N,N-diethylaminoethyl, 3-N,N-diethylaminopropyl, 2-(N-methylpiperazinyl)ethyl, 2-(N-acetylpiperazinyl)ethyl, 2-morpholinylethyl, 2-thiomorpholinylethyl,2-piperidylethyl, 2-hydroxyethyl, 3-hydroxypropyl, 2-methoxyethyl, N-methyl-4-piperidyl, N-ethyl-4-piperidyl, N-propyl-4-piperidyl, N-isopropyl-4-piperidyl, N-hydroxyethyl-4-piperidyl, N-cyanomethyl-4-piperidyl, N-(2-cyanoethyl)-4-piperidyl, 3-N,N-dimethylaminocyclopentyl, 3-N,N-diethylaminocyclopentyl, 3-N,N-dipropylaminocyclopentyl, 3-N,N-isopropylaminocyclopentyl, 4-aminocyclohexyl, 4-N,N-dimethylaminocyclohexyl, 4-N,N-diethylaminocyclohexyl, 4-N,N-dipropylaminocyclohexyl, 4-N,N-diisopropylaminocyclohexyl, 4-hydroxycyclohexyl, 4-methoxycyclohexyl, 4-ethoxycyclohexyl, 4-isopropoxycyclohexyl;
3) 1-methyl-3-pyrrolyl, 1-ethyl-3-pyrrolyl, 1-isopropyl-3-pyrrolyl, 1-cyclopropyl-3-pyrrolyl, 1-methyl-3-pyrazolyl, 1-ethyl-3-pyrazolyl, 1-isopropyl-3-pyrazolyl, 1-cyclopropyl-3-pyrazolyl, 1-hydroxyethyl-3-pyrazolyl, 1-methyl-4-pyrazolyl, 1-ethyl-4-pyrazolyl, 1-isopropyl-4-pyrazolyl, 1-cyclopropyl-4-pyrazolyl, 1-hydroxyethyl-4-pyrazolyl, 1,3-dimethyl-5-pyrazolyl, 1-methyl-4-imidazolyl, 3-methyl-5-isoxazolyl;
4)

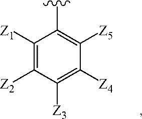

Z₁, Z₂, Z₃, Z₄, Z₅ each are independently selected from the following groups, and Z₁, Z₂, Z₃, Z₄, Z₅ are not hydrogen at the same time:
   (1) H, F, Cl, Br, I, nitro, cyano,
   (2) C₁—C₆ alkyl, C₁—C₆ alkoxy, C₁—C₆ oxygen-containing alkyl, C₁—C₆ fluorine-containing alkyl, C₁—C₆ fluorine-containing alkoxy, C₃—C₆ cycloalkyl, C₂—C₆ alkenyl,
   (3) piperazinyl, N-methylpiperazinyl, N-ethylpiperazinyl, N-n-propylpiperazinyl, N-isopropylpiperazinyl, N-cyclopropylpiperazinyl, N-(2-hydroxyethyl)piperazinyl, N-(2-N,N-dimethylaminoethyl)piperazinyl, N-(2-N,N-diethylaminoethyl)piperazinyl, N-(2-cyanoethyl)piperazinyl, N-cyanomethylpiperazinyl, N-(3-hydroxypropyl)piperazinyl, N-(3-N,N-dimethylaminopropyl)piperazinyl, N-(3-N,N-diethylaminopropyl)piperazinyl,
   (4) morpholinyl, 2,6-dimethylmorpholinyl, thiomorpholinyl,
   (5) tetrahydropyrrolyl, 3-(N,N-dimethylamino)tetrahydropyrrolyl, 3-(N,N-diethylamino)tetrahydropyrrolyl,
   (6) imidazolyl, 4-methyl-1-imidazolyl,
   (7) piperidyl, 4-N,N-dimethylaminopiperidyl, 4-N,N-diethylaminopiperidyl, 4-N,N-diisopropylaminopiperidyl, 4-hydroxypiperidyl, 4-methoxypiperidyl, 4-ethoxypiperidyl, 4-(N-methylpiperazinyl-1-)piperidyl, 4-(N-ethylpiperazinyl-1-)piperidyl, 4-(N-isopropylpiperazinyl-1-)piperidyl, 4-(N-(2-hydroxyethyl)piperazinyl-1-)piperidyl, 4-(N-(2-cyanoethyl)piperazinyl-1-)piperidyl, 4-(N-(3-hydroxypropyl)piperazinyl-1-)piperidyl, 4-(N-(2-N,N-dimethylaminoethyl)piperazinyl-1-)piperidyl, 4-(N-(2-N,N-diethylethyl)piperazinyl-1-)piperidyl, 4-(N-(3-N,N-dimethylpropyl)piperazinyl-1-)piperidyl, 4-(N-(3-N,N-diethylpropyl)piperazinyl-1-)piperidyl, 4-(tetrahydropyrrolyl-1-)piperidyl, 4-(3-N,N-dimethylaminotetrahydropyrrolyl-1-)piperidyl, 4-(3-N,N-diethylaminotetrahydropyrrolyl-1-)piperidyl,
   (8) piperidin-4-yl, N-methyl-4-piperidyl, N-ethyl-4-piperidyl, N-isopropyl-4-piperidyl, N-(2-hydroxyethyl)-4-piperidyl, N-(2-N,N-dimethylaminoethyl)-4-piperidyl, N-(2-N,N-diethylaminoethyl)-4-piperidyl, N-(2-cyanoethyl)-4-piperidyl, N-(3-hydroxypropyl)-4-piperidyl, N-(3-N,N-dimethylaminopropyl)-4-piperidyl, N-(3-N,N-diethylaminopropyl)-4-piperidyl, N-(3-cyanopropyl)-4-piperidyl, N-cyanomethylene-4-piperidyl,
   (9) amino, N,N-dimethylamino, N,N-diethylamino, N,N-di-n-propylamino, N,N-diisopropylamino, 2-N,N-dimethylaminoethylamino, 3-N,N-dimethylaminopropylamino, 3-N,N-diethylaminopropylamino, 3-N,N-diisopropylaminopropylamino, N-methylpiperidyl-4-amino, N-ethylpiperidyl-4-amino, N-n-propylpiperidyl-4-amino, N-isopropylpiperidyl-4-amino, acetamido, propionamido, 2-(N,N-dimethylamino)acetamido, 2-hydroxyacetamido, 2-methoxyacetamido, methylsulfamido, ethylsulfamido, n-propylsulfamido, isopropylsulfamido, cyclopropylsulfamido, N-methyl-N-(N-methyl-3-tetrahydropyrrolyl)amino, N-(N-methyl-3-tetrahydropyrrolyl)amino, N-methyl-N-(2-N,N-dimethylamino)ethylamino,
   (10) 4-methyl-1-piperazinylmethylene, 4-ethyl-1-piperazinylmethylene, 4-propyl-1-piperazinylmethylene, 4-isopropyl-1-piperazinylmethylene, 4-(2-hydroxyethyl)-1-piperazinylmethylene, 4-(2-N,N-dimethylaminoethyl)-1-piperazinylmethylene, piperidyl-1-methylene, 4-(N,N-dimethylamino)-1-piperidylmethylene, 4-hydroxypiperidyl-1-methylene, tetrahydropyrrolyl-1-methylene, 3-(N,N-dimethylamino)-1-tetrahydropyrrolylmethylene, benzyl, cyclopropylmethylene, cyclobutylmethylene, cyclopentylmethylene, cyclohexylmethylene,
   (11) aminoformyl, methylaminoformyl, N,N-dimethylaminoformyl, ethylaminoformyl, aminoacetyl, methylaminoacetyl, 2-(N,N-dimethylamino)acetyl, ethylaminoacetyl, tetrahydropyrrolyl-1-formyl, 3-(N,N-dimethylamino)tetrahydropyrrolyl-1-formyl, 3-(N,N-diethylamino)tetrahydropyrrolyl-1-formyl, morpholinyl-4-formyl, thiomorpholinyl-4-formyl, 2,6-dimethylmorpholinyl-4-formyl, piperidyl-1-formyl, 4-hydroxypiperidyl-1-formyl, 4-(N,N-dimethylamino)piperidyl-1-formyl, 4-(N,N-diethylamino)piperidyl-1-formyl, 4-(N-methyl-1-piperazinyl)piperidyl-1-formyl, 4-(N-ethyl-1-piperazinyl)piperidyl-1-formyl, N-methylpiperazinyl-1-formyl, N-ethylpiperazinyl-1-formyl, N-isopropylpiperazinyl-1-formyl, N-(2-hydroxyethyl)piperazinyl-1-formyl, N-(2-N,N-dimethylaminoethyl)piperazinyl-1-formyl, N-(2-N,N-diethylaminoethyl)piperazinyl-1-formyl, 4-(N-methyl-4-piperidyl)piperazinyl-1-formyl, 4-(N-ethyl-4-piperidyl)piperazinyl-1-formyl,
   (12) hydroxysulfonyl, aminosulfonyl, methylaminosulfonyl, ethylaminosulfonyl, propylaminosulfonyl, isopropylaminosulfonyl, cyclopropylaminosulfonyl, cyclobutylaminosulfonyl, cyclopentylaminosulfonyl, cyclohexylaminosulfonyl, tetrahydropyrrolyl-1-sulfonyl, 3-(N,N-dimethylamino)tetrahydropyrrolyl-1-sulfonyl, 3-(N,N-diethylamino)tetrahydropyrrolyl-1-sulfonyl, morpholinyl-4-sulfonyl, thiomorpholinyl-4-sulfonyl, 2,6-dimethylmorpholinyl-4-sulfonyl, piperidyl-1-sulfonyl, 4-hydroxypiperidyl-1-sulfonyl, 4-(N,N-dimethylamino)piperidyl-1-sulfonyl, 4-(N,N-diethylamino)piperidyl-1-sulfonyl, N-methylpiperazinyl-1-sulfonyl, N-ethylpiperazinyl-1-sulfonyl, N-isopropylpiperazinyl-1-sulfonyl, N-(2-hydroxyethyl)piperazinyl-1-sulfonyl, N-(2-N,N-dimethylaminoethyl)piperazinyl-1-sulfonyl, N-(2-N,N-diethylaminoethyl)piperazinyl-1-sulfonyl,
   (13) aminoformamido, methylaminoformamido, ethylaminoformamido, propylaminoformamido, isopropylaminoformamido;
5)

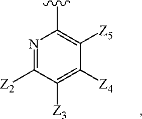

wherein Z₂, Z₃, Z₄, Z₅ are the same as defined in 4), and Z₂, Z₃, Z₄, Z₅ are not hydrogen at the same time;
6)

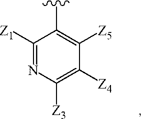

wherein Z₁, Z₃, Z₄, Z₅ are the same as defined in 4), and Z₁, Z₃, Z₄, Z₅ are not hydrogen at the same time;
7)

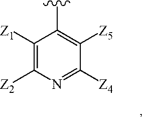

wherein Z₁, Z₂, Z₄, Z₅ are the same as defined in 4), and Z₁, Z₂, Z₄, Z₅ may be hydrogen at the same time;
8)

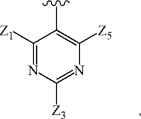

wherein Z₁, Z₃, Z₅ are the same as defined in 4), and Z₁, Z₃, Z₅ are not hydrogen at the same time;
9)

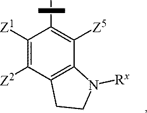

wherein Z₁, Z₂, Z₅ are the same as defined in 4), and Z₁, Z₂, Z₅ may be hydrogen at the same time, R^{X} is selected from the same substituents as described above for Z¹;
10)

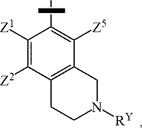

wherein Z₁, Z₂, Z₅ are the same as defined in 4), and Z₁, Z₂, Z₅ may be hydrogen at the same time, R^{y} is selected from the same substituents as described above for Z¹;
11)

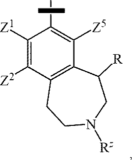

wherein Z₁, Z₂, Z₅ are the same as defined in 4), and Z₁, Z₂, Z₅ may be hydrogen at the same time, R^{z}, R each are independently selected from the same substituents as described above for Z¹.

In some embodiments, R¹ is selected from:
1) H, C₁-C₆ alkyl, C₃-C₈ cycloalkyl;
2) 2-N,N-dimethylaminoethyl, 3-N,N-dimethylaminopropyl, 2-N,N-diethylaminoethyl, 3-N,N-diethylaminopropyl, 2-hydroxyethyl, 3-hydroxypropyl, N-methyl-4-piperidyl, N-ethyl-4-piperidyl, N-propyl-4-piperidyl, N-isopropyl-4-piperidyl, 4-aminocyclohexyl, 4-N,N-dimethylaminocyclohexyl, 4-N,N-diethylaminocyclohexyl, 4-N,N-dipropylaminocyclohexyl, 4-N,N-diisopropylaminocyclohexyl;
3) 1-methyl-3-pyrazolyl, 1-ethyl-3-pyrazolyl, 1-isopropyl-3-pyrazolyl, 1-cyclopropyl-3-pyrazolyl, 1-methyl-4-pyrazolyl, 1-ethyl-4-pyrazolyl, 1-isopropyl-4-pyrazolyl, 1-cyclopropyl-4-pyrazolyl, 1,3-dimethyl-5-pyrazolyl, 1-methyl-4-imidazolyl;
4)

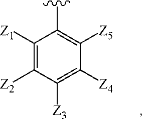

wherein Z₁, Z₂, Z₃, Z₄, Z₅ each are independently selected from the following groups, and Z₁, Z₂, Z₃, Z₄, Z₅ are not hydrogen at the same time:
   (1) H, F, Cl, Br, I, nitro, cyano,
   (2) C₁-C₆ alkyl, C₁-C₆ alkoxy, C₁-C₆ fluorine-containing alkyl, C₁-C₆ fluorine-containing alkoxy, C₂-C₆ alkenyl,
   (3) piperazinyl, N-methylpiperazinyl, N-ethylpiperazinyl, N-n-propylpiperazinyl, N-isopropylpiperazinyl,
   (4) morpholinyl, 2,6-dimethylmorpholinyl, thiomorpholinyl,
   (5) 3-(N,N-dimethylamino)tetrahydropyrrolyl, 3-(N,N-diethylamino)tetrahydropyrrolyl,
   (6) 4-N,N-dimethylaminopiperidyl, 4-N,N-diethylaminopiperidyl, 4-N,N-diisopropylaminopiperidyl, 4-hydroxypiperidyl, 4-(N-methylpiperazinyl-1-)piperidyl, 4-(N-ethylpiperazinyl-1-)piperidyl, 4-(N-isopropylpiperazinyl-1-)piperidyl, 4-(tetrahydropyrrolyl-1-)piperidyl,
   (7) piperidin-4-yl, N-methyl-4-piperidyl, N-ethyl-4-piperidyl, N-isopropyl-4-piperidyl,
   (8) aminoformamido, methylaminoformamido, ethylaminoformamido, propylaminoformamido, isopropylaminoformamido, 2-(N,N-dimethylamino)acetamido, cyclopropylsulfamido, 2-N,N-dimethylaminoethylamino, 3-N,N-dimethylaminopropylamino, 3-N,N-diethylaminopropylamino, 3-N,N-diisopropylaminopropylamino, N-methyl-N-(2-N,N-dimethylamino)ethylamino,
   (9) 4-methyl-1-piperazinylmethylene, 4-ethyl-1-piperazinylmethylene, 4-propyl-1-piperazinylmethylene, 4-isopropyl-1-piperazinylmethylene, benzyl, cyclopropylmethylene, cyclobutylmethylene, cyclopentylmethylene, cyclohexylmethylene,
   (10) 4-(N-methyl-1-piperazinyl)piperidyl-1-formyl, 4-(N-ethyl-1-piperazinyl)piperidyl-1-formyl, N-methylpiperazinyl-1-formyl, N-ethylpiperazinyl-1-formyl, N-isopropylpiperazinyl-1-formyl,
   (11) cyclopropylaminosulfonyl, cyclobutylaminosulfonyl, cyclopentylaminosulfonyl, cyclohexylam inosulfonyl, N-methylpiperazinyl-1-sulfonyl, N-ethylpiperazinyl-1-sulfonyl, N-isopropylpiperazinyl-1-sulfonyl;
5)

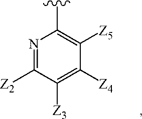

wherein Z₂, Z₃, Z₄, Z₅ each are independently selected from H, C₁-C₆ alkyl, C₁-C₆ alkoxy, piperazinyl, N-methylpiperazinyl, N-ethylpiperazinyl, N-n-propylpiperazinyl, or N-isopropylpiperazinyl, and Z₂, Z₃, Z₄, Z₅ are not hydrogen at the same time;
6)

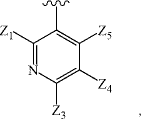

wherein Z₁, Z₃, Z₄, Z₅ each are independently selected from H, C₁—C₆ alkyl, C₁—C₆ alkoxy, N-(N-methyl-3-tetrahydropyrrolyl)amino, N-methyl-N-(N-methyl-3-tetrahydropyrrolyl)amino, N-methylpiperazinyl, N-ethylpiperazinyl, N-n-propylpiperazinyl, N-isopropylpiperazinyl, 4-(N-methylpiperazinyl-1-)piperidyl, 4-(N-ethylpiperazinyl-1-)piperidyl, or 4-(N-isopropylpiperazinyl-1-)piperidyl, and Z₁, Z₃, Z₄, Z₅ are not hydrogen at the same time;
7)

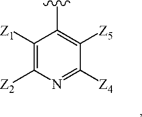

wherein Z₁, Z₂, Z₄, Z₅ each are independently selected from H, or C₁—C₆ alkyl, and Z₁, Z₂, Z₄, Z₅ may be hydrogen at the same time;
8)

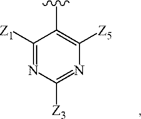

wherein Z₁, Z₃, Z₅ each are independently selected from H, C₁-C₆ alkyl, C₁-C₆ alkoxy, N-methylpiperazinyl, N-ethylpiperazinyl, N-n-propylpiperazinyl, or N-isopropylpiperazinyl, and Z₁, Z₃, Z₅ are not hydrogen at the same time;
9)

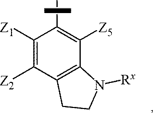

wherein Z₁, Z₂, Z₅ each are independently selected from H, C₁-C₆ alkyl, or C₁-C₆ alkoxy, and Z₁, Z₂, Z₅ may be hydrogen at the same time, R^{X} is selected from aminoformyl, methylaminoformyl, N,N-dimethylaminoformyl, ethylaminoformyl, aminoacetyl, methylaminoacetyl, 2-(N,N-dimethylamino)acetyl, or ethylaminoacetyl;
10)

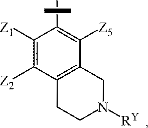

wherein Z₁, Z₂, Z₅ each are independently selected from H, F, Cl, Br, I, C₁-C₆ alkyl, C₁-C₆ alkoxy, C₁-C₆ fluorine-containing alkyl, or C₁-C₆ fluorine-containing alkoxy, and Z₁, Z₂, Z₅ may be hydrogen at the same time, R^{y} is C₁-C₆ alkyl;
11)

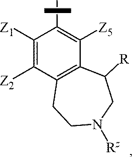

wherein Z₁, Z₂, Z₅ each are independently selected from H, or C₁-C₆ alkyl, and Z₁, Z₂, Z₅ may be hydrogen at the same time, R^{z}, R each are independently selected from C₁-C₆ alkyl.

In some embodiments, R¹ is selected from:
1) cyclopropyl, 2-N,N-dimethylaminoethyl, 2-hydroxyethyl, N-methyl-4-piperidyl, 4-aminocyclohexyl, 1-methyl-3-pyrazolyl, 1-methyl-4-pyrazolyl, 1,3-dimethyl-5-pyrazolyl, 1-methyl-4-imidazolyl;
2)

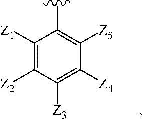

wherein:
   Z₁, Z₂, Z₃, Z₄, Z₅ each are independently selected from the following groups, and Z₁, Z₂, Z₃, Z₄, Z₅ are not hydrogen at the same time:
      H, methyl, ethyl, methoxy, ethoxy, isopropoxy, trifluoromethyl, trifluoromethoxy, vinyl, N-methylpiperazinyl, morpholinyl, 3-(N,N-dimethylamino)tetrahydropyrrolyl, 4-N,N-dimethylaminopiperidyl, 4-hydroxypiperidyl, 4-(N-methylpiperazinyl-1-)piperidyl, 4-(tetrahydropyrrolyl-1-)piperidyl, N-methyl-4-piperidyl, 2-(N,N-dimethylamino)acetamido, cyclopropylsulfamido, N-methyl-N-(2-N,N-dimethylamino)ethylamino, 4-methyl-1-piperazinylmethylene, benzyl, cyclopropylmethylene, 4-(N-methyl-1-piperazinyl)piperidyl-1-formyl, N-methylpiperazinyl-1-formyl, cyclopropylaminosulfonyl, or N-methylpiperazinyl-1-sulfonyl;
      preferably, Z₁, Z₂, Z₅ each are independently selected from H, methoxy, ethoxy, isopropoxy, trifluoromethyl, or trifluoromethoxy; and one of Z₃ and Z₄ is selected from the following groups, and the other is H, methyl, ethyl, vinyl, benzyl or cyclopropylmethylene: N-methylpiperazinyl, morpholinyl, 3-(N,N-dimethylamino)tetrahydropyrrolyl, 4-N,N-dimethylaminopiperidyl, 4-hydroxypiperidyl, 4-(N-methylpiperazinyl-1-)piperidyl, 4-(tetrahydropyrrolyl-1-)piperidyl, N-methyl-4-piperidyl, 2-(N,N-dimethylamino)acetamido, cyclopropylsulfamido, N-methyl-N-(2-N,N-dimethylamino)ethylamino, 4-methyl-1-piperazinylmethylene, 4-(N-methyl-1-piperazinyl)piperidyl-1-formyl, N-methylpiperazinyl-1-formyl, cyclopropylaminosulfonyl, or N-methylpiperazinyl-1-sulfonyl;
3)

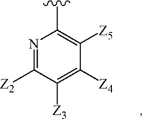

wherein Z₂, Z₃, Z₄, Z₅ each are independently selected from H, methoxy, or N-methylpiperazinyl, and Z₂, Z₃, Z₄, Z₅ are not hydrogen at the same time;
4)

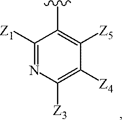

wherein:
   Z₁, Z₃, Z₄, Z₅ each are independently selected from H, methyl, ethyl, methoxy, N-(N-methyl-3-tetrahydropyrrolyl)am ino, N-methyl-N-(N-methyl-3-tetrahydropyrrolyl)amino, N-methylpiperazinyl, or 4-(N-methylpiperazinyl-1-)piperidyl, and Z₁, Z₃, Z₄, Z₅ are not hydrogen at the same time;
   preferably, Z₁, Z₄, Z₅ each are independently selected from H, methyl, ethyl, or methoxy, Z₃ is methyl, N-(N-methyl-3-tetrahydropyrrolyl)amino, N-methyl-N-(N-methyl-3-tetrahydropyrrolyl)amino, N-methylpiperazinyl or 4-(N-methylpiperazinyl-1-)piperidyl;
5)

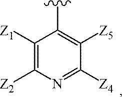

wherein Z₁, Z₂, Z₄, Z₅ are H;
6)

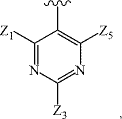

wherein Z₁, Z₃, Z₅ each are independently selected from H, methoxy, or N-methylpiperazinyl, and Z₁, Z₃, Z₅ are not hydrogen at the same time;
7)

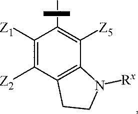

wherein Z₁, Z₂, Z₅ each are independently selected from H, or methoxy, R^{X} is 2-(N,N-dimethylamino)acetyl;
8)

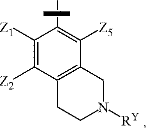

wherein Z₁, Z₂, Z₅ each are independently selected from H, F, Cl, methyl, methoxy, trifluoromethyl, or trifluoromethoxy, R^{y} is methyl;
9)

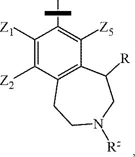

wherein Z₁, Z₂, Z₅ are H, R^{z}, R are methyl.

In some embodiments, R², R³ each are independently selected from C₁-C₆ alkyl, C₁-C₆ fluorine-containing alkyl, or C₃-C₆ cycloalkyl.

In some embodiments, R², R³ each are independently selected from methyl, trifluoromethyl, ethyl, isopropyl, or cyclopropyl.

In some embodiments, R² is selected from methyl, trifluoromethyl, ethyl, isopropyl, or cyclopropyl, R³ is selected from methyl, ethyl, isopropyl, or cyclopropyl.

In some embodiments, R⁵ is selected from F, Cl, Br, trifluoromethyl, cyano, or nitro.

In the ninth aspect of the present invention, the present invention provides a compound having the general formula (II-1) or (II-2):

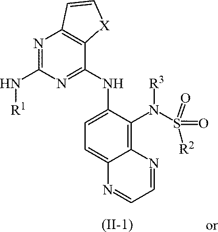

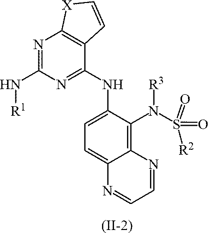

or a stereoisomer, a prodrug, a pharmaceutically acceptable salt or a pharmaceutically acceptable solvate thereof, wherein:
each X is independently selected from NH, O, or S;
each R¹ is independently selected from:
   1) 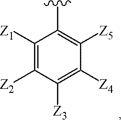
   wherein Z₁, Z₂, Z₃, Z₄, Z₅ each are independently selected from the following groups, and Z₁, Z₂, Z₃, Z₄, Z₅ are not hydrogen at the same time:
      (1) H, F, Cl, Br, I, nitro, cyano,
      (2) C₁—C₆ alkyl, C₁—C₆ alkoxy, C₁—C₆ oxygen-containing alkyl, C₁—C₆ fluorine-containing alkyl, C₁—C₆ fluorine-containing alkoxy, C₃—C₆ cycloalkyl, C₂—C₆ alkenyl,
      (3) 2-N,N-dimethylaminoethoxy, 3-N,N-dimethylaminopropoxy, 2-(N-methylpiperazinyl)ethoxy, 2-(N-acetylpiperazinyl)ethoxy, 2-morpholinylethoxy, 2-thiomorpholinylethoxy, 2-piperidylethoxy, 3-N,N-dimethylaminopropoxy, 3-N,N-diethylaminopropoxy, 3-N,N-diisopropylaminopropoxy, 3-(N-methylpiperazinyl)propoxy, 3-(N-acetylpiperazinyl)propoxy, 3-morpholinylpropoxy, 3-thiomorpholinylpropoxy, 3-piperidylpropoxy, 2-pyridylmethoxy, 3-pyridylmethoxy, 4-pyridylmethoxy, phenylmethoxy, monohalo-substituted phenylmethoxy, gem-dihalo-substituted phenylmethoxy, hetero-dihalo-substituted phenylmethoxy,
      (4) piperazinyl, N-methylpiperazinyl, N-ethylpiperazinyl, N-n-propylpiperazinyl, N-isopropylpiperazinyl, N-cyclopropylpiperazinyl, N-(2-hydroxyethyl)piperazinyl, N-(2-N,N-dimethylaminoethyl)piperazinyl, N-(2-N,N-diethylaminoethyl)piperazinyl, N-(2-cyanoethyl)piperazinyl, N-cyanomethylpiperazinyl, N-(3-hydroxypropyl)piperazinyl, N-(3-N,N-dimethylaminopropyl)piperazinyl, N-(3-N,N-diethylaminopropyl)piperazinyl, N-(N-methyl-4-piperidyl)piperazinyl, N-(N-ethyl-4-piperidyl)piperazinyl, N-t-butoxycarbonylpiperazinyl, N-acetylpiperazinyl, N-propionylpiperazinyl, N-isobutyrylpiperazinyl, N-cyclopropylformylpiperazinyl, N-methylsulfonylpiperazinyl, N-ethylsulfonylpiperazinyl, N-n-propylsulfonylpiperazinyl, N-isopropylsulfonylpiperazinyl, N-cyclopropylsulfonylpiperazinyl, 2-oxo-piperazin-4-yl,
      (5) morpholinyl, 2,6-dimethylmorpholinyl, thiomorpholinyl,
      (6) tetrahydropyrrolyl, 3-(N,N-dimethylamino)tetrahydropyrrolyl, 3-(N,N-diethylamino)tetrahydropyrrolyl,
      (7) imidazolyl, 4-methyl-1-imidazolyl,
      (8) piperidyl, 4-N,N-dimethylaminopiperidyl, 4-N,N-diethylaminopiperidyl, 4-N,N-diisopropylaminopiperidyl, 4-hydroxypiperidyl, 4-methoxypiperidyl, 4-ethoxypiperidyl, 4-(N-methylpiperazinyl-1-)piperidyl, 4-(N-ethylpiperazinyl-1-)piperidyl, 4-(N-isopropylpiperazinyl-1-)piperidyl, 4-(N-acetylpiperazinyl-1-)piperidyl, 4-(N-t-butoxycarbonylpiperazinyl-1-)piperidyl, 4-(N-methylsulfonylpiperazinyl-1-)piperidyl, 4-(N-(2-hydroxyethyl)piperazinyl-1-)piperidyl, 4-(N-(2-cyanoethyl)piperazinyl-1-)piperidyl, 4-(N-(3-hydroxypropyl)piperazinyl-1-)piperidyl, 4-(N-(2-N,N-dimethylaminoethyl)piperazinyl-1-)piperidyl, 4-(N-(2-N,N-diethylethyl)piperazinyl-1-)piperidyl, 4-(N-(3-N,N-dimethylpropyl)piperazinyl-1-)piperidyl, 4-(N-(3-N,N-diethylpropyl)piperazinyl-1-)piperidyl, 4-(tetrahydropyrrolyl-1-)piperidyl, 4-(3-N,N-dimethylaminotetrahydropyrrolyl-1-)piperidyl, 4-(3-N,N-diethylaminotetrahydropyrrolyl-1-)piperidyl,
      (9) piperidin-4-yl, N-methyl-4-piperidyl, N-ethyl-4-piperidyl, N-isopropyl-4-piperidyl, N-acetyl-4-piperidyl, N-t-butoxycarbonyl-4-piperidyl, N-methylsulfonyl-4-piperidyl, N-(2-hydroxyethyl)-4-piperidyl, N-(2-N,N-dimethylaminoethyl)-4-piperidyl, N-(2-N,N-diethylaminoethyl)-4-piperidyl, N-(2-cyanoethyl)-4-piperidyl, N-(3-hydroxypropyl)-4-piperidyl, N-(3-N,N-dimethylaminopropyl)-4-piperidyl, N-(3-N,N-diethylaminopropyl)-4-piperidyl, N-(3-cyanopropyl)-4-piperidyl, N-cyanomethylene-4-piperidyl,
      (10) amino, N,N-dimethylamino, N,N-diethylamino, N,N-di-n-propylamino, N,N-diisopropylamino, 2-N,N-dimethylaminoethylamino, 2-morpholinylethylamino, 2-(N-methylpiperazinyl)ethylamino, 3-N,N-dimethylaminopropylamino, 3-N,N-diethylaminopropylamino, 3-N,N-diisopropylaminopropylamino, 3-morpholinylpropylamino, 3-(N-methylpiperazinyl)propylamino, N-methylpiperidyl-4-amino, N-ethylpiperidyl-4-amino, N-n-propylpiperidyl-4-amino, N-isopropylpiperidyl-4-amino, acetamido, propionamido, 2-(N,N-dimethylamino)acetamido, 2-hydroxyacetamido, 2-methoxyacetamido, methylsulfamido, ethylsulfamido, n-propylsulfamido, isopropylsulfamido, cyclopropylsulfamido, N-methyl-N-(N-methyl-3-tetrahydropyrrolyl)amino, N-(N-methyl-3-tetrahydropyrrolyl)amino, N-methyl-N-(2-N,N-dimethylamino)ethylamino,
      (11) aminomethylene, N,N-dimethylaminomethylene, N,N-diethylaminomethylene, 4-methyl-1-piperazinylmethylene, 4-ethyl-1-piperazinylmethylene, 4-propyl-1-piperazinylmethylene, 4-isopropyl-1-piperazinylmethylene, 4-(2-hydroxyethyl)-1-piperazinylmethylene, 4-(2-N,N-dimethylaminoethyl)-1-piperazinylmethylene, piperidyl-1-methylene, 4-(N,N-dimethylamino)-1-piperidylmethylene, 4-hydroxypiperidyl-1-methylene, tetrahydropyrrolyl-1-methylene, 3-(N,N-dimethylamino)-1-tetrahydropyrrolylmethylene, benzyl, cyclopropylmethylene, cyclobutylmethylene, cyclopentylmethylene, cyclohexylmethylene,
      (12) aminoformyl, methylaminoformyl, N,N-dimethylaminoformyl, ethylaminoformyl, cyclopropylaminoformyl, cyclobutylaminoformyl, cyclopentylaminoformyl, cyclohexylaminoformyl, aminoacetyl, methylaminoacetyl, 2-(N,N-dimethylamino)acetyl, ethylam inoacetyl, cyclopropylam inoacetyl, cyclobutylam inoacetyl, cyclopentylaminoacetyl, cyclohexylaminoacetyl, tetrahydropyrrolyl-1-formyl, 3-(N,N-dimethylamino)tetrahydropyrrolyl-1-formyl, 3-(N,N-diethylamino)tetrahydropyrrolyl-1-formyl, morpholinyl-4-formyl, thiomorpholinyl-4-formyl, 2,6-dimethylmorpholinyl-4-formyl, piperidyl-1-formyl, 4-hydroxypiperidyl-1-formyl, 4-(N,N-dimethylamino)piperidyl-1-formyl, 4-(N,N-diethylamino)piperidyl-1-formyl, 4-(N-methyl-1-piperazinyl)piperidyl-1-formyl, 4-(N-ethyl-1-piperazinyl)piperidyl-1-formyl, N-methylpiperazinyl-1-formyl, N-ethylpiperazinyl-1-formyl, N-isopropylpiperazinyl-1-formyl, N-(2-hydroxyethyl)piperazinyl-1-formyl, N-(2-N,N-dimethylaminoethyl)piperazinyl-1-formyl, N-(2-N,N-diethylaminoethyl)piperazinyl-1-formyl, 4-(N-methyl-4-piperidyl)piperazinyl-1-formyl, 4-(N-ethyl-4-piperidyl)piperazinyl-1-formyl,
      (13) hydroxyformyl, methoxyformyl, ethoxyformyl, propoxyformyl, isopropoxyformyl, n-butoxyformyl, iso-butoxyformyl, t-butoxyformyl,
      (14) hydroxysulfonyl, aminosulfonyl, methylaminosulfonyl, ethylaminosulfonyl, propylaminosulfonyl, isopropylaminosulfonyl, cyclopropylaminosulfonyl, cyclobutylaminosulfonyl, cyclopentylaminosulfonyl, cyclohexylaminosulfonyl, tetrahydropyrrolyl-1-sulfonyl, 3-(N,N-dimethylamino)tetrahydropyrrolyl-1-sulfonyl, 3-(N,N-diethylamino)tetrahydropyrrolyl-1-sulfonyl, morpholinyl-4-sulfonyl, thiomorpholinyl-4-sulfonyl, 2,6-dimethylmorpholinyl-4-sulfonyl, piperidyl-1-sulfonyl, 4-hydroxypiperidyl-1-sulfonyl, 4-(N,N-dimethylamino)piperidyl-1-sulfonyl, 4-(N,N-diethylamino)piperidyl-1-sulfonyl, 4-(N-methyl-1-piperazinyl)piperidyl-1-sulfonyl, 4-(N-ethyl-1-piperazinyl)piperidyl-1-sulfonyl, N-methylpiperazinyl-1-sulfonyl, N-ethylpiperazinyl-1-sulfonyl, N-isopropylpiperazinyl-1-sulfonyl, N-(2-hydroxyethyl)piperazinyl-1-sulfonyl, N-(2-N,N-dimethylaminoethyl)piperazinyl-1-sulfonyl, N-(2-N,N-diethylaminoethyl)piperazinyl-1-sulfonyl, 4-(N-methyl-4-piperidyl)piperazinyl-1-sulfonyl, 4-(N-ethyl-4-piperidyl)piperazinyl-1-sulfonyl,
      (15) aminoformamido, methylaminoformamido, ethylaminoformamido, propylam inoformamido, isopropylam inoformamido, cyclopropylam inoformamido, cyclobutylaminoformamido, cyclopentylaminoformamido, piperidyl-1-formamido, 4-hydroxypiperidyl-1-formamido, 4-N,N-dimethylpiperidyl-1-formamido, 4-N,N-diethylpiperidyl-1-formamido, tetrahydropyrrolyl-1-formamido, 3-N,N-dimethyltetrahydropyrrolyl-1-formamido, 3-N,N-diethyltetrahydropyrrolyl-1-formamido, N-methylpiperazinyl-1-formamido, N-ethylpiperazinyl-1-formamido, N-acetylpiperazinyl-1-formamido, N-t-butoxycarbonylpiperazinyl-1-formamido, N-(2-hydroxyethyl)piperazinyl-1-formamido, N-(2-cyanoethyl)piperazinyl-1-formamido, N-(2-N,N-dimethylaminoethyl)piperazinyl-1-formamido, N-(2-N,N-diethylaminoethyl)piperazinyl-1-formamido, N-(3-hydroxypropyl)piperazinyl-1-formamido, N-(3-N,N-dimethylpropyl)piperazinyl-1-formamido, N-(3-N,N-diethylpropyl)piperazinyl-1-formamido, morpholinyl-1-formamido, 3,5-dimethylmorpholinyl-1-formamido, 4-(tetrahydropyrrolyl)piperidyl-1-formamido, 4-(N-methyl-1-piperazinyl)piperidyl-1-formamido, 4-(N-ethyl-1-piperazinyl)piperidyl-1-formamido, 4-(N-acetyl-1-piperazinyl)piperidyl-1-formamido, N-(N-methyl-4-piperidyl)piperazinyl-1-formamido;
   2) 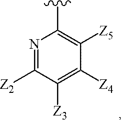
   wherein Z₂, Z₃, Z₄, Z₅ are the same as defined in 1), and Z₂, Z₃, Z₄, Z₅ are not hydrogen at the same time;
   3) 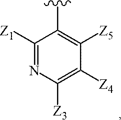
   wherein Z₁, Z₃, Z₄, Z₅ are the same as defined in 1), and Z₁, Z₃, Z₄, Z₅ are not hydrogen at the same time;
   4) 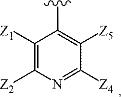
   wherein Z₁, Z₂, Z₄, Z₅ are the same as defined in 1), and Z₁, Z₂, Z₄, Z₅ may be hydrogen at the same time;
   5) 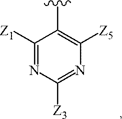
   wherein Z₁, Z₃, Z₅ are the same as defined in 1), and Z₁, Z₃, Z₅ are not hydrogen at the same time;
R², R³ each are independently selected from:
   H, C₁-C₆ alkyl, C₁-C₆ fluorine-containing alkyl, C₃-C₆ cycloalkyl, or C₃-C₆ fluorine-containing cycloalkyl.

In some embodiments, each R¹ is independently selected from:
1)

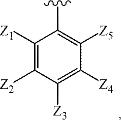

wherein Z₁, Z₂, Z₃, Z₄, Z₅ each are independently selected from the following groups, and Z₁, Z₂, Z₃, Z₄, Z₅ are not hydrogen at the same time:
   (1) H, F, Cl, Br, I, nitro, cyano,
   (2) C₁—C₆ alkyl, C₁—C₆ alkoxy, C₁—C₆ oxygen-containing alkyl, C₁—C₆ fluorine-containing alkyl, C₁—C₆ fluorine-containing alkoxy, C₃—C₆ cycloalkyl, C₂—C₆ alkenyl,
   (3) piperazinyl, N-methylpiperazinyl, N-ethylpiperazinyl, N-n-propylpiperazinyl, N-isopropylpiperazinyl, N-cyclopropylpiperazinyl, N-(2-hydroxyethyl)piperazinyl, N-(2-N,N-dimethylaminoethyl)piperazinyl, N-(2-N,N-diethylaminoethyl)piperazinyl, N-(2-cyanoethyl)piperazinyl, N-cyanomethylpiperazinyl, N-(3-hydroxypropyl)piperazinyl, N-(3-N,N-dimethylaminopropyl)piperazinyl, N-(3-N,N-diethylaminopropyl)piperazinyl,
   (4) morpholinyl, 2,6-dimethylmorpholinyl, thiomorpholinyl,
   (5) tetrahydropyrrolyl, 3-(N,N-dimethylamino)tetrahydropyrrolyl, 3-(N,N-diethylamino)tetrahydropyrrolyl,
   (6) imidazolyl, 4-methyl-1-imidazolyl,
   (7) piperidyl, 4-N,N-dimethylaminopiperidyl, 4-N,N-diethylaminopiperidyl, 4-N,N-diisopropylaminopiperidyl, 4-hydroxypiperidyl, 4-methoxypiperidyl, 4-ethoxypiperidyl, 4-(N-methylpiperazinyl-1-)piperidyl, 4-(N-ethylpiperazinyl-1-)piperidyl, 4-(N-isopropylpiperazinyl-1-)piperidyl, 4-(N-(2-hydroxyethyl)piperazinyl-1-)piperidyl, 4-(N-(2-cyanoethyl)piperazinyl-1-)piperidyl, 4-(N-(3-hydroxypropyl)piperazinyl-1-)piperidyl, 4-(N-(2-N,N-dimethylaminoethyl)piperazinyl-1-)piperidyl, 4-(N-(2-N,N-diethylethyl)piperazinyl-1-)piperidyl, 4-(N-(3-N,N-dimethylpropyl)piperazinyl-1-)piperidyl, 4-(N-(3-N,N-diethylpropyl)piperazinyl-1-)piperidyl, 4-(tetrahydropyrrolyl-1-)piperidyl, 4-(3-N,N-dimethylaminotetrahydropyrrolyl-1-)piperidyl, 4-(3-N,N-diethylaminotetrahydropyrrolyl-1-)piperidyl,
   (8) piperidin-4-yl, N-methyl-4-piperidyl, N-ethyl-4-piperidyl, N-isopropyl-4-piperidyl, N-(2-hydroxyethyl)-4-piperidyl, N-(2-N,N-dimethylaminoethyl)-4-piperidyl, N-(2-N,N-diethylaminoethyl)-4-piperidyl, N-(2-cyanoethyl)-4-piperidyl, N-(3-hydroxypropyl)-4-piperidyl, N-(3-N,N-dimethylaminopropyl)-4-piperidyl, N-(3-N,N-diethylaminopropyl)-4-piperidyl, N-(3-cyanopropyl)-4-piperidyl, N-cyanomethylene-4-piperidyl,
   (9) amino, N,N-dimethylamino, N,N-diethylamino, N,N-di-n-propylamino, N,N-diisopropylamino, 2-N,N-dimethylaminoethylamino, 3-N,N-dimethylaminopropylamino, 3-N,N-diethylaminopropylamino, 3-N,N-diisopropylaminopropylamino, N-methylpiperidyl-4-amino, N-ethylpiperidyl-4-amino, N-n-propylpiperidyl-4-amino, N-isopropylpiperidyl-4-amino, acetamido, propionamido, 2-(N,N-dimethylamino)acetamido, 2-hydroxyacetamido, 2-methoxyacetamido, methylsulfamido, ethylsulfamido, n-propylsulfamido, isopropylsulfamido,cyclopropylsulfamido, N-methyl-N-(N-methyl-3-tetrahydropyrrolyl)amino, N-(N-methyl-3-tetrahydropyrrolyl)amino, N-methyl-N-(2-N,N-dimethylamino)ethylamino,
   (10) 4-methyl-1-piperazinylmethylene, 4-ethyl-1-piperazinylmethylene, 4-propyl-1-piperazinylmethylene, 4-isopropyl-1-piperazinylmethylene, 4-(2-hydroxyethyl)-1-piperazinylmethylene, 4-(2-N,N-dimethylaminoethyl)-1-piperazinylmethylene, piperidyl-1-methylene, 4-(N,N-dimethylamino)-1-piperidylmethylene, 4-hydroxypiperidyl-1-methylene, tetrahydropyrrolyl-1-methylene, 3-(N,N-dimethylamino)-1-tetrahydropyrrolylmethylene, benzyl, cyclopropylmethylene, cyclobutylmethylene, cyclopentylmethylene, cyclohexylmethylene,
   (11) aminoformyl, methylaminoformyl, N,N-dimethylaminoformyl, ethylaminoformyl, aminoacetyl, methylaminoacetyl, 2-(N,N-dimethylamino)acetyl, ethylaminoacetyl, tetrahydropyrrolyl-1-formyl, 3-(N,N-dimethylamino)tetrahydropyrrolyl-1-formyl, 3-(N,N-diethylamino)tetrahydropyrrolyl-1-formyl, morpholinyl-4-formyl, thiomorpholinyl-4-formyl, 2,6-dimethylmorpholinyl-4-formyl, piperidyl-1-formyl, 4-hydroxypiperidyl-1-formyl, 4-(N,N-dimethylamino)piperidyl-1-formyl, 4-(N,N-diethylamino)piperidyl-1-formyl, 4-(N-methyl-1-piperazinyl)piperidyl-1-formyl, 4-(N-ethyl-1-piperazinyl)piperidyl-1-formyl, N-methylpiperazinyl-1-formyl, N-ethylpiperazinyl-1-formyl, N-isopropylpiperazinyl-1-formyl, N-(2-hydroxyethyl)piperazinyl-1-formyl, N-(2-N,N-dimethylaminoethyl)piperazinyl-1-formyl, N-(2-N,N-diethylaminoethyl)piperazinyl-1-formyl, 4-(N-methyl-4-piperidyl)piperazinyl-1-formyl, 4-(N-ethyl-4-piperidyl)piperazinyl-1-formyl,
   (12) hydroxysulfonyl, aminosulfonyl, methylaminosulfonyl, ethylaminosulfonyl, propylaminosulfonyl, isopropylaminosulfonyl, cyclopropylaminosulfonyl, cyclobutylaminosulfonyl, cyclopentylaminosulfonyl, cyclohexylaminosulfonyl, tetrahydropyrrolyl-1-sulfonyl, 3-(N,N-dimethylamino)tetrahydropyrrolyl-1-sulfonyl, 3-(N,N-diethylamino)tetrahydropyrrolyl-1-sulfonyl, morpholinyl-4-sulfonyl, thiomorpholinyl-4-sulfonyl, 2,6-dimethylmorpholinyl-4-sulfonyl, piperidyl-1-sulfonyl, 4-hydroxypiperidyl-1-sulfonyl, 4-(N,N-dimethylamino)piperidyl-1-sulfonyl, 4-(N,N-diethylamino)piperidyl-1-sulfonyl, N-methylpiperazinyl-1-sulfonyl, N-ethylpiperazinyl-1-sulfonyl, N-isopropylpiperazinyl-1-sulfonyl, N-(2-hydroxyethyl)piperazinyl-1-sulfonyl, N-(2-N,N-dimethylaminoethyl)piperazinyl-1-sulfonyl, N-(2-N,N-diethylaminoethyl)piperazinyl-1-sulfonyl,
   (13) aminoformamido, methylaminoformamido, ethylaminoformamido, propylaminoformamido, isopropylaminoformamido;
2)

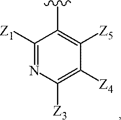

wherein Z₁, Z₃, Z₄, Z₅ are the same as defined in 1), and Z₁, Z₃, Z₄, Z₅ are not hydrogen at the same time.

In some embodiments, each R¹ is independently selected from:
1)

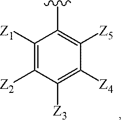

wherein Z₁, Z₂, Z₃, Z₄, Z₅ each are independently selected from the following groups, and Z₁, Z₂, Z₃, Z₄, Z₅ are not hydrogen at the same time:
   (1) H, F, Cl, Br, I, nitro, cyano,
   (2) C₁—C₆ alkyl, C₁—C₆ alkoxy, C₁—C₆ fluorine-containing alkoxy, C₂—C₆ alkenyl,
   (3) piperazinyl, N-methylpiperazinyl, N-ethylpiperazinyl, N-n-propylpiperazinyl, N-isopropylpiperazinyl,
   (5) 3-(N,N-dimethylamino)tetrahydropyrrolyl, 3-(N,N-diethylamino)tetrahydropyrrolyl,
   (6) 4-N,N-dimethylaminopiperidyl, 4-N,N-diethylaminopiperidyl, 4-N,N-diisopropylaminopiperidyl, 4-(N-methylpiperazinyl-1-)piperidyl, 4-(N-ethylpiperazinyl-1-)piperidyl, 4-(N-isopropylpiperazinyl-1-)piperidyl,
   (7) benzyl, cyclopropylmethylene, cyclobutylmethylene, cyclopentylmethylene, cyclohexylmethylene;
2)

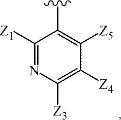

wherein Z₁, Z₃, Z₄, Z₅ each are independently selected from H, C₁-C₆ alkyl, C₁-C₆ alkoxy, 4-(N-methylpiperazinyl-1-)piperidyl, 4-(N-ethylpiperazinyl-1-)piperidyl, or 4-(N-isopropylpiperazinyl-1-)piperidyl, and Z₁, Z₃, Z₄, Z₅ are not hydrogen at the same time.

In some embodiments, each R¹ is independently selected from:
1)

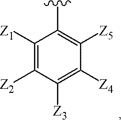

wherein:
   Z₁, Z₂, Z₃, Z₄, Z₅ each are independently selected from the following groups, and Z₁, Z₂, Z₃, Z₄, Z₅ are not hydrogen at the same time:
      H, methyl, ethyl, methoxy, trifluoromethoxy, vinyl, N-methylpiperazinyl, 3-(N,N-dimethylamino)tetrahydropyrrolyl, 4-N,N-dimethylaminopiperidyl, 4-(N-methylpiperazinyl-1-)piperidyl, benzyl, or cyclopropylmethylene;
      preferably, Z₁, Z₂, Z₅ each are independently selected from H, methoxy, or trifluoromethoxy, Z₄ is H, methyl, ethyl, vinyl, benzyl or cyclopropylmethylene, Z₃ is N-methylpiperazinyl, 3-(N,N-dimethylamino)tetrahydropyrrolyl, 4-N,N-dimethylamino piperidyl or 4-(N-methylpiperazinyl-1-)piperidyl;
2)

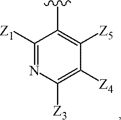

wherein:
   Z₁, Z₃, Z₄, Z₅ each are independently selected from H, methyl, ethyl, methoxy, or 4-(N-methylpiperazinyl-1-)piperidyl, and Z₁, Z₃, Z₄, Z₅ are not hydrogen at the same time;
   preferably, Z₁, Z₄, Z₅ each are independently selected from H, methyl, ethyl, or methoxy, Z₃ is 4-(N-methylpiperazinyl-1-)piperidyl.

In some embodiments, R², R³ each are independently selected from H, or C₁-C₆ alkyl.

In some embodiments, R², R³ each are independently selected from methyl.

In some embodiments, the pharmaceutically acceptable salt is an inorganic acid salt or an organic acid salt, wherein the inorganic acid salt is hydrochloride, hydrobromide, hydroiodide, nitrate, bicarbonate and carbonate, sulfate or phosphate; the organic acid salt is formate, acetate, propionate, benzoate, maleate, fumarate, succinate, tartrate, citrate, ascorbate, a-ketoglutarate, a-glycerophosphate, alkyl sulfonate or aryl sulfonate; preferably, the alkyl sulfonate is methyl sulfonate or ethyl sulfonate; the aryl sulfonate is benzenesulfonate or p-toluenesulfonate.

Unless otherwise indicated, the above groups and substituents have the ordinary meanings in the field of medicinal chemistry.

The term “C₁-C₆ alkyl” refers to any straight-chain or branched-chain group containing 1 to 6 carbon atoms, such as methyl, ethyl, n-propyl, isopropyl, n-butyl, isobutyl, t-butyl, sec-butyl, n-pentyl, tert-amyl, n-hexyl and the like.

The term “C₁-C₃ alkyl” refers to any straight-chain or branched-chain group containing 1 to 3 carbon atoms, such as methyl, ethyl, n-propyl, isopropyl and the like.

The term “oxygen-containing alkyl” refers to a group in which the alkyl skeleton is substituted by one or more alkoxy groups, for example, methoxyethyl, methoxyethoxymethyl and the like.

For example, C₁-C₆ oxygen-containing alkyl refers to a group in which a C₁-C₆ alkyl skeleton is substituted by one or more C₁-C₆ alkoxy groups, for example, methoxyethyl, methoxyethoxymethyl and the like. Similarly, C₁-C₃ oxygen-containing alkyl refers to a group in which a C₁-C₃ alkyl skeleton is substituted by one or more C₁-C₆ alkoxy groups.

The term “C₂-C₆ alkenyl” refers to any straight-chain or branched-chain group containing 2 to 6 carbon atoms and containing at least one carbon-carbon double bond, such as vinyl, 1-propenyl, 2-propenyl and the like.

The term “Cs-Cs cycloalkyl” refers to a hydrocarbon having a 3- to 8-membered monocylic system of saturated ring, and the C₃-C₈ cycloalkyl may be cyclopropyl, cyclobutyl, cyclopentyl, cyclohexyl and the like.

The term “C₃-C₆ cycloalkyl” refers to a hydrocarbon having a 3- to 6-membered monocylic system of saturated ring, and the C₃-C₆ cycloalkyl may be cyclopropyl, cyclobutyl, cyclopentyl, cyclohexyl and the like.

The term “cyano” refers to -CN residue.

The term “nitro” refers to —NO₂ group.

The terms “alkoxy”, “cycloalkoxy” and derivatives thereof refer to any of the above groups (such as C₁-C₆ alkyl, C₁-C₃ alkyl, etc.), or cycloalkyl (e.g., C₃-C₆ cycloalkyl), which is attached to the remainder of molecules through oxygen atom (-O-).

The term “heteroaryl” refers to an aromatic heterocyclic ring, which is usually a 5-, 6-, 7-, or 8-membered heterocyclic ring having from 1 to 3 heteroatoms selected from N, O or S; a heteroaryl ring may be optionally further fused or attached to aromatic or non-aromatic carbocyclic rings or heterocyclic rings. Non-limiting examples of the heteroaryl group are, for example, pyridyl, pyrazinyl, pyrimidinyl, pyridazinyl, indolyl, imidazolyl, thiazolyl, isothiazolyl, thioxazolyl, pyrrolyl, phenyl-pyrrolyl, furyl, phenyl-furyl, oxazolyl, isoxazolyl, pyrazolyl, thienyl, benzofuryl, benzothienyl, benzo(1,3-dioxolanyl) (benzodioxolyl), isoindolinyl, benzoimidazolyl, indazolyl, quinolyl, isoquinolyl, 1,2,3-triazolyl, 1-phenyl-1,2,3-triazolyl, 2,3-indolinyl, 2,3-dihydrobenzofuryl, 2,3-dihydrobenzothienyl, benzopyranyl, 2,3-dihydrobenzoxazinyl, 2,3-dihydroquinoxalinyl and the like.

The term “heterocyclyl” (also referred to as “heterocycloalkyl”) refers to 3-, 4-, 5-, 6- and 7-membered saturated or partially unsaturated carbocyclic rings, wherein one or more carbon atoms are replaced by heteroatoms such as nitrogen, oxygen and sulfur. Non-limiting examples of the heterocyclic group are, for example, pyranyl, pyrrolidinyl, pyrrolinyl, imidazolinyl, imidazolidinyl, pyrazolidinyl, pyrazolinyl, thiazolinyl, thiazolidinyl, dihydrofuryl, tetrahydrofuryl, 1,3-dioxolanyl, piperidinyl, piperazinyl, morpholine, morpholinyl, tetrahydropyrrolyl, thiomorpholinyl and the like.

For example, “6-memebred heterocyclyl” refers to 6-membered saturated or partially unsaturated carbocyclic rings, wherein one or more carbon atoms are replaced by heteroatoms such as nitrogen, oxygen and sulfur. Non-limiting examples of the 6-membered heterocyclyl are, for example, pyranyl, piperidinyl, piperazinyl, morpholine, morpholinyl, thiomorpholinyl and the like.

“5-Memebred heterocyclyl” refers to 5-membered saturated or partially unsaturated carbocyclic rings, wherein one or more carbon atoms are replaced by heteroatoms such as nitrogen, oxygen and sulfur. Non-limiting examples of the 5-membered heterocyclyl are, for example, pyrrolidinyl, pyrrolinyl, imidazolinyl, imidazolidinyl, pyrazolidinyl, pyrazolinyl, thiazolinyl, thiazolidinyl, 1,3-dioxolanyl and the like.

The term “optionally substituted heterocyclyl” refers to that the above “heterocyclyl” is substituted with one or more “C₁-C₆ alkyl”, “C₁-C₃ alkyl”, “C₃-C₆ cycloalkyl” and the like.

“Fluorine-containing alkyl” refers to groups in which an alkyl skeleton is substituted with one or more fluorine groups, such as monofluoromethyl, difluoroethyl, trifluoromethyl and the like.

The term “C₁-C₆ fluorine-containing alkyl” refers to groups in which a C₁-C₆ alkyl skeleton is substituted with one or more fluorine groups, such as monofluoromethyl, difluoroethyl, trifluoromethyl and the like.

Similarly, the term “C₁-C₃ fluorine-containing alkyl” refers to groups in which a C₁-C₃ alkyl skeleton is substituted with one or more fluorine groups, such as monofluoromethyl, difluoroethyl, trifluoromethyl and the like.

The term “C₁-C₆ heteroatom-containing alkyl” refers to groups in which one or more carbon atoms in a C₁-C₆ alkyl skeleton is replaced by one or more heteroatoms, such as N, O or S, such as

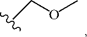

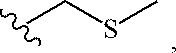

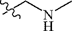

and the like.

The term “Cs-Cs heteroatom-containing cycloalkyl” refers to groups in which one or more carbon atoms in a C₃-C₈ cycloalkyl skeleton is replaced by one or more heteroatoms, such as N, O or S, such as pyrrolidinyl, imidazolidinyl, pyrazolidinyl, thiazolidinyl, piperidinyl, piperazinyl, morpholine, morpholinyl, thiomorpholinyl and the like.

The term “C₁-C₆ acyl” refers to —C(═O)—H and —C(═O)—Ci—C₅ alkyl, such as formyl, acetyl, propionyl, butyryl and the like.

The term “sulfonyl” refers to —S(═O)₂—.

The term “C₁—C₆ alkylsulfonyl” refers to —S(═O)₂—C₁—C₆ alkyl, such as methylsulfonyl, ethylsulfonyl, propylsulfonyl, butylsulfonyl and the like.

The terms “alkoxy”, “cycloalkoxy” and derivatives thereof refer to any of the above groups (such as C₁-C₆ alkyl, C₁-C₃ alkyl, etc.), or cycloalkyl (e.g., C₃-C₆ cycloalkyl), which is attached to the remainder of molecules through oxygen atom (—O—).

From all of the above description, it will be apparent to those skilled in the art that any group whose name is a compound name, such as “fluorine-containing oxygen-containing alkyl”, shall mean to conventionally construct from the moiety that is derived, such as the oxygen-containing alkyl substituted by the fluorine group, wherein the alkyl is as defined above, and similarly, a “fluorine-containing alkoxy” is further exemplified. Moreover, for example, the “arylamino” shall mean to conventionally construct from the moiety that is derived, such as the amino substituted by the aryl, wherein the aryl is as defined above. Similarly, the meaning of “heteroarylamino” should be understood. Similarly, the meanings of “hydroxysulfonyl”, “aminosulfonyl” and the like should be understood.

Similarly, any term such as alkylamino, dialkylamino, alkoxycarbonyl, alkoxycarbonylamino, heterocyclylcarbonyl, heterocyclylcarbonylamino, cycloalkyloxycarbonyl, alkoxyformyl and the like includes groups, wherein alkyl, alkoxy, aryl, C₃-C₇ cycloalkyl and heterocyclyl moieties are as defined above.

In the general formula

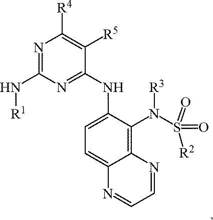

the term “R⁴, R⁵ and carbon atoms linked thereto together form

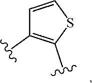

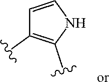

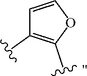

means the formation of the general formula

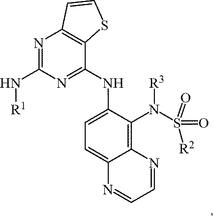

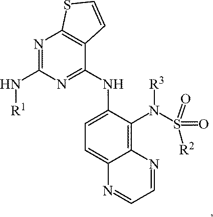

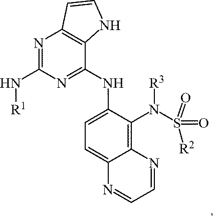

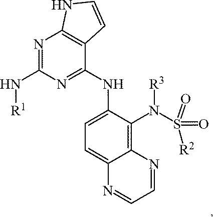

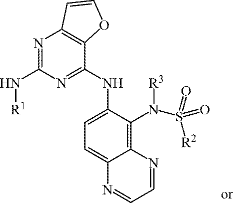

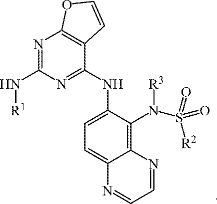

The term “each R¹ is independently selected from” means that each qroup represented by R¹ may be the same or different. For example, “in the formula

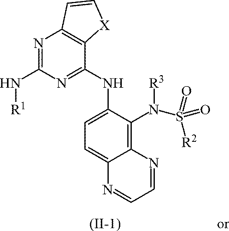

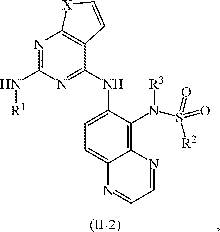

each R¹ is independent selected from H or methyl”, which means that R¹ in the formula (ll-1) is selected from H or methyl, and R¹ in the formula (ll-2) is selected from H or methyl.

The term “R², R³ each are independently selected from” means that groups represented by each R², each R³ or R² and R³ may be the same or different. For example, “in

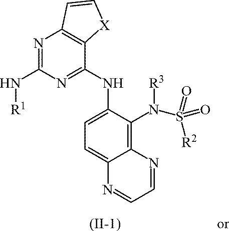

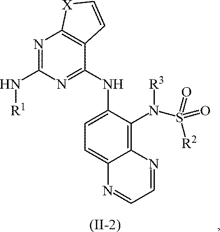

R², R³ each are independently selected from H or methyl”, which means that in the formula (ll-1), R² is selected from H or methyl, and in the formula (ll-1), R³ is selected from H or methyl; in the formula (ll-2), R² is selected from H or methyl, and in the formula (ll-2), R³ is selected from H or methyl.

According to the invention, unless otherwise indicated, any of the above groups may be optionally substituted by one or more groups at any free position thereof, for example by 1-6 groups, which are independently selected from: halogen atom, nitro, oxo (═O), cyano, C₁-C₆ alkyl, polyfluoroalkyl, polyfluoroalkoxy, alkenyl, alkynyl, hydroxyalkyl, hydroxyalkylamino, hydroxyheterocyclyl, aryl, aryl-alkyl, heteroaryl, heteroaryl-alkyl, heterocyclyl, heterocyclyl-alkyl, C₃-C₇ cycloalkyl, cycloalkyl-alkyl, alkyl-aryl, alkyl-heteroaryl, alkyl-heterocyclyl, alkyl-cycloalkyl, alkyl-aryl-alkyl, alkyl-heteroaryl-alkyl, alkyl-heterocyclyl-alkyl, alkyl-cycloalkyl-alkyl, alkyl-heterocyclyl-heterocyclyl, heterocyclyl-heterocyclyl, heterocyclyl-alkyl-heterocyclyl, heterocyclyl-alkylamino, alkyl-heterocyclyl-alkyl-amino, hydroxy, alkoxy, aryloxy, heterocyclyloxy, alkyl-heterocyclyloxy, methylenedioxy, alkylcarbonyloxy, arylcarbonyloxy, cycloalkenyloxy, heterocyclyl carbonyloxy, alkylene aminooxy, carboxy, alkoxycarbonyl, aryloxycarbonyl, cycloalkyloxycarbonyl, heterocyclyl oxycarbonyl, amino, ureido, alkylamino, amino-alkylamino, dialkylamino, dialkylamino-heterocyclyl, dialkylamino-alkylamino, arylamino, arylalkylamino, diarylamino, heterocyclylamino, alkyl-heterocyclylamino, alkyl-heterocyclylcarbonyl, formylamino, alkylcarbonylamino, arylcarbonylam ino, heterocyclylcarbonylam ino, alkyl-heterocyclylcarbonylam ino, aminocarbonyl, alkylaminocarbonyl, dialkylaminocarbonyl, arylaminocarbonyl, heterocyclylaminocarbonyl, alkoxycarbonylamino, alkoxycarbonylamino-alkylamino, alkoxycarbonylheterocyclyl-alkylamino, alkoxy-aryl-alkyl, hydroxyamino-carbonyl, alkoxyimino, alkylsulfonylamino, arylsulfonylamino, heterocyclylsulfonylamino, formyl, alkylcarbonyl, arylcarbonyl, cycloalkylcarbonyl, heterocyclylcarbonyl, alkylsulfonyl, arylsulfonyl, aminosulfonyl, alkylaminosulfonyl, dialkylaminosulfonyl, arylaminosulfonyl, heterocyclyl aminosulfonyl, arylthio, alkylthio, phosphonate and alkylphosphonate.

Further, if appropriate, each of the above substituents may be further substituted by one or more of the above-exemplified groups.

The term “oxygen-containing substituted or unsubstituted five- to seven-membered ring” or “nitrogen-containing substituted or unsubstituted five- to seven-membered ring” refers to 5-, 6-, or 7-membered saturated or partially unsaturated carbocyclic rings, wherein one or more carbon atoms are replaced by oxygen or nitrogen. Their non-limiting examples are, for example, pyran, pyrrolidine, pyrroline, imidazoline, imidazolidine, pyrazolidine, pyrazoline, dihydrofuran, tetrahydrofuran, 1,3-dioxolane, piperidine, piperazine, morpholine, tetrahydropyrrolyl, hexamethylene imine and the like.

The term “1 to 2 heteroatom-containing substituted or unsubstituted three- to seven-membered ring” refers to 3-, 4-, 5-, 6-, or 7-membered saturated or partially unsaturated carbocyclic rings, wherein one or two carbon atoms are replaced by heteroatoms such as oxygen, nitrogen or sulfur. Their non-limiting examples are, for example, pyran, pyrrolidine, pyrroline, imidazoline, imidazolidine, pyrazolidine, pyrazoline, dihydrofuran, tetrahydrofuran, 1,3-dioxolane, piperidine, piperazine, morpholine, tetrahydropyrrolyl, hexamethylene imine and the like.

As used herein, unless otherwise indicated, the term “prodrug” refers to a derivative that can be hydrolyzed, oxidized or otherwise reacted under biological conditions (in vitro or in vivo) to provide a compound of the invention. Prodrugs can become active compounds only by carrying out the reaction under biological conditions, or they are inactive in their non-reacted form. Prodrugs can be generally prepared using known methods, for example, those methods described in Burger’s Medicinal Chemistry and Drug Discovery (1995) 172-178, 949-982 (Manfred E. Wolff, ed. 5^{th} edition).

As used herein, examples of the term “pharmaceutically acceptable salt of a compound of formula (A), (l), (ll-1) or (ll-2)” are addition salts formed with organic acids capable of forming pharmaceutically acceptable anions, including, but not limited to, formate, acetate, propionate, benzoate, maleate, fumarate, succinate, tartrate, citrate, ascorbate, a-ketoglutarate, α-glycerophosphate, alkyl sulfonate or aryl sulfonate; preferably, the alkyl sulfonate is methyl sulfonate or ethyl sulfonate; the aryl sulfonate is benzenesulfonate or p-toluenesulfonate. Suitable inorganic salts also can be formed, including, but not limited to, hydrochloride, hydrobromide, hydroiodide, nitrate, bicarbonate and carbonate, sulfate or phosphate.

Pharmaceutically acceptable salts can be obtained using standard procedures well known in the art, for example, by reacting a sufficient amount of a basic compound with a suitable acid that provides a pharmaceutically acceptable anion.

The term “treatment” as used herein generally refers to obtaining the desired pharmacological and/or physiological effect. The effect may be preventive according to complete or partial prevention of disease or its symptoms; and/or may be therapeutic according to partial or complete stabilization or cure of disease and/or side effects due to the disease. The term “treatment” as used herein encompasses any treatment on a patient’s disease, including: (a) preventing the disease or symptom that occurs in a patient who is susceptible to the disease or symptom but not yet diagnosed to suffer from the disease; (b) suppressing symptoms of the disease, i.e., stopping its development; or (c) relieving symptoms of the disease, i.e., causing degeneration of the disease or symptom.

According to a specific embodiment of the present invention relating to the compound, a stereoisomer thereof, a prodrug thereof, or a pharmaceutically acceptable salt thereof or a pharmaceutically acceptable solvate thereof, the compound is one of the compounds described in the examples below.

In another aspect, the present invention provides a pharmaceutical composition comprising the compound according to any one of the above embodiments, a stereoisomer thereof, a prodrug thereof, or a pharmaceutically acceptable salt thereof or a pharmaceutically acceptable solvate thereof, and optionally a pharmaceutically acceptable carrier, diluent or excipient.

In some embodiments, the pharmaceutical composition further comprises a EGFR monoclonal antibody.

In some embodiments, the EGFR monoclonal antibody is Cetuximab or biosimilars thereof.

The term “biosimilars” refers to antibody products having the same sequence to Cetuximab and having consistent physicochemical properties, biological activities, and clinical safety and efficiencies to the Cetuximab.

Methods for preparing a pharmaceutical composition comprising a certain amount of an active ingredient, are known or are obvious for a person skilled in the art according to the contents as disclosed in the invention. For example, as described in REMINGTON’S PHARMACEUTICAL SCIENCES, Martin, E.W., ed., Mack Publishing Company, 19^{th} ed. (1995), methods for preparing a pharmaceutical composition comprise incorporating a suitable pharmaceutically acceptable excipient, carrier, diluent, etc.

The known methods for preparing a pharmaceutical preparation according to the invention include the conventional mixing, dissolving or freeze-drying methods. The compound according to the invention can be used to prepare into a pharmaceutical composition, which is administered to a patient by various routes suitable for the selected administration mode, for example, oral, or parenteral route (intravenous, intramuscular, topical, or subcutaneous route).

Therefore, the compound of the invention in combination with a pharmaceutically acceptable carrier (such as an inert diluent or an assimilable edible carrier) can be administered systemically, e.g., orally. They can be encapsulated into a hard or soft shell gelatin capsule, or pressed into a tablet. For the treatment by oral administration, an active compound may be combined with one or more excipients, and be used in a form of a deglutible tablet, a buccal tablet, a troche, a capsule, an elixir, a suspension, a syrup, a wafer, etc. The composition and preparation shall comprise at least 0.1% of an active compound. The ratio of the active compound in the composition or the preparation can be varied certainly, and the ratio may account for about 1 wt% to about 99 wt% of a given unit dosage form. In such a therapeutically active composition, the active compound is in an amount sufficient to obtain an effective dosage level.

A tablet, a troche, a pill, a capsule, and the like may include: a binder, such as tragacanth gum, arabic gum, maize starch or gelatin; an excipient, such as dicalcium phosphate; a disintegrant, such as maize starch, potato starch, and alginic acid etc.; a lubricant, such as magnesium stearate; and a sweeting agent, such as sucrose, fructose, lactose or aspartame; or a flavoring agent, such as peppermint, winter green oil or cherry flavor. When the unit dosage form is a capsule, in addition to the above types of materials, it may comprise a liquid carrier, such as vegetable oil or polyethylene glycol. Various other materials may be present as a coating or change the physical form of a solid unit dosage form in other manners. For example, a tablet, a pill or a capsule may be coated with gelatin, wax, shellac or sugar etc. A syrup or elixir may comprise an active compound, sucrose or fructose is used as a sweeting agent, methyl p-hydroxybenzoate or propyl p-hydroxybenzoate is used as a preservative, a dye and a flavoring agent (such as a cherry flavor or an orange flavor). Certainly, any material for preparing any unit dosage form should be pharmaceutically acceptable and be substantively not toxic in its applied amount. In addition, an active compound may be incorporated into a sustained release preparation and a sustained release device.

An active compound may also be administered intravenously or intraperitoneally by infusion or injection. An aqueous solution of an active compound or a salt thereof may be prepared, optionally, by mixing it with a non-toxic surfactant. A dispersible formulation in glycerol, liquid polyethylene glycol, glycerin triacetate and a mixture thereof and in oil may also be prepared. Under the common conditions of storage and use, the preparations may comprise a preservative in order to suppress the growth of microbes.

A pharmaceutical dosage form suitable for injection or infusion may include a sterile aqueous solution or a dispersible formulation or a sterile powder comprising an active ingredient (optionally encapsulated into a liposome) of an immediate preparation such as a solution or a dispersible formulation suitable for sterile injection or infusion. Under all the conditions, the final dosage form shall be sterile, liquid and stable under the production and storage conditions. A liquid carrier may be a solution or a liquid disperse medium, including, for example, water, ethanol, polyols (such as glycerol, propylene glycol, and liquid macrogol, etc.), vegetable oil, a non-toxic glyceride and a suitable mixture thereof. A suitable fluidity may be retained, for example, by the formation of liposome, by retaining the desired particle size in the presence of a dispersing agent, or by using a surfactant. The effect of suppressing microbes can be obtained by various antibacterial agents and antifungal agents (such as paraben, chlorbutol, phenol, sorbic acid, and thiomersal, etc.). In many conditions, an isotonizing agent, such as sugar, buffer agent or NaCl, is preferably comprised. By the use of a composition of delayed absorbents (e.g., aluminium monostearate and gelatin), an extended absorption of an injectable composition can be obtained.

A sterile injectable solution can be prepared by mixing a desired amount of an active compound in a suitable solvent with the desired various other ingredients as listed above, and then performing filtration and sterilization. In the case of a sterile powder for the preparation of a sterile injectable solution, the preferred preparation methods are vacuum drying and freeze drying techniques, which will result in the production of the powder of the active ingredient and any other desired ingredient present in the previous sterile filtration solution.

A useful solid carrier includes crushed solid (such as talc, clay, microcrystalline cellulose, silicon dioxide, and aluminum oxide etc.). A useful liquid carrier includes water, ethanol or ethylene glycol or water-ethanol/ethylene glycol mixture, in which the compound of the invention may be dissolved or dispersed in an effective amount, optionally, with the aid of a non-toxic surfactant. An adjuvant (such as a flavor) and an additional antimicrobial agent may be added to optimize the property for a given use.

A thickener (such as synthetic polymer, fatty acid, fatty acid salt and ester, fatty alcohol, modified cellulose or modified inorganic material) may also be used with a liquid carrier to form a coatable paste, gel, ointment, soap and the like, and be directly applied to the skin of a user.

A therapeutically required amount of a compound or an active salt or derivative thereof not only depends on the specific salt selected, but also depends on the administration mode, the nature of the disease to be treated and the age and state of a patient, and finally depends on the decision made by an attending physician or a clinical physician.

Above preparation may be present in a unit dosage form, which is a physical dispersion unit comprising a unit dose, suitable for administration to a human body and other mammalian body. A unit dosage form may be capsule(s) or tablet(s). Depending on the particular treatment involved, the amount of an active ingredient in a unit dose may be varied or adjusted between about 0.1 and about 1000 mg or more.

In addition, the present invention further includes use of various new drug dosage forms such as milk liposomes, microspheres and nanospheres, for example, medicaments prepared with the use of a particulate dispersion system including polymeric micelles, nanoemulsions, submicroemulsions, microcapsules, microspheres, liposomes and niosomes (also known as nonionic surfactant vesicles), etc.

In another aspect, the present invention further provides a preparation method of the compound according to any of the above embodiments, comprising the following steps:

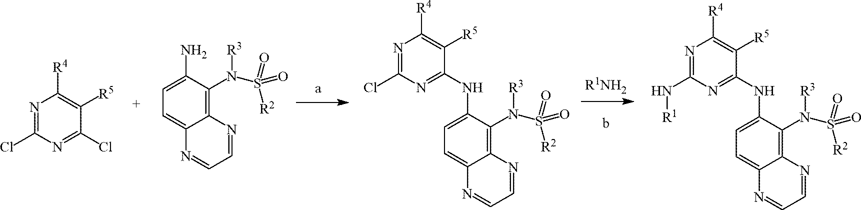

reaction conditions: (a) a nucleophilic substitution reaction of heteroaryl chloride with amine compound under acidic or alkaline condition; (b) a coupling reaction of carbon-nitrogen bond formation of heteroaryl chloride and amine compound catalyzed by metal palladium, or a nucleophilic substitution reaction of heteroaryl chloride with amine compound under acidic condition;
wherein the heteroaryl chloride comprises the following types (“P” represents protective groups, for example: benzyloxycarbonyl (Cbz), t-butoxycarbonyl (BOC), 9-fluorenylmethoxycarbonyl (FMOC), benzyl (Bn), p-methoxybenzyl (PMB), phenylsulfonyl, p-methylphenylsulfonyl, 2-(trimethylsilyl)ethoxymethyl (SEM), acetyl (Ac), tribenzyl derivative protective groups): 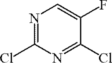 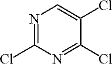 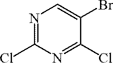 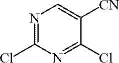 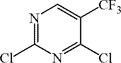 [SMILES: FC(F)(F)c1cnc(Cl)nc1Cl] 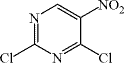 [SMILES: O=[N+]([O-])c1cnc(Cl)nc1Cl] 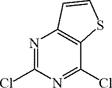 [SMILES: Clc1nc(Cl)c2sccc2n1] 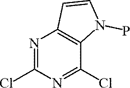 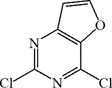 [SMILES: Clc1nc(Cl)c2occc2n1] 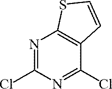 [SMILES: Clc1nc(Cl)c2ccsc2n1] 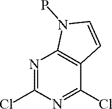 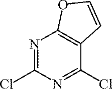 [SMILES: Clc1nc(Cl)c2ccoc2n1] 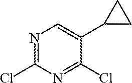 [SMILES: Clc1ncc(C2CC2)c(Cl)n1] 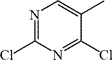 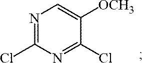
   the amine compound is selected from substituted or unsubstituted 5- or 6-membered heterocyclic amines, aniline and derivatives thereof, C₁—C₆ alkylamine, C₃—C₇ cycloalkylamine, C₁—C₆ oxygen-containing alkylamine or C₃—C₇ oxygen-containing cycloalkylamine, see above for details;
   the metal palladium catalyst is selected from palladium acetate, tetrakis(triphenylphosphine) palladium, bistriphenylphosphine palladium dichloride, [1,1′-bis(diphenylphosphino)ferrocene] palladium dichloride or tris(dibenzylideneacetone) dipalladium; the alkaline condition refers to a condition in which any of the following substances exists: triethylamine, diisopropylethylamine, pyridine, sodium bicarbonate, sodium carbonate, potassium carbonate, cesium carbonate, lithium hydroxide, sodium hydroxide, potassium hydroxide, sodium hydride, or potassium hydride; the acidic condition refers to a condition in which any of the following substances exists: acetic acid, trifluoroacetic acid, hydrochloric acid, methanesulfonic acid, p-toluene sulfonic acid, or camphorsulfonic acid.

In another aspect, the present invention further provides a method of preparing the compound according to any of the above embodiments, comprising the following steps:

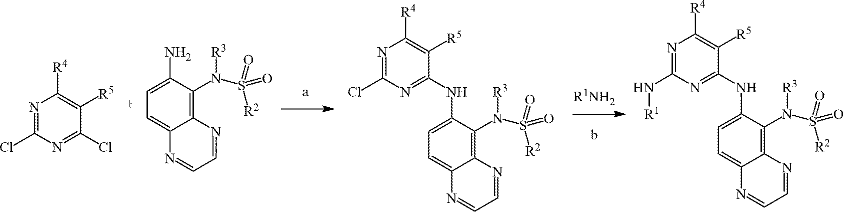

reaction conditions are as follows:
(a) a nucleophilic substitution reaction under acidic or alkaline condition;
(b) a coupling reaction catalyzed by metal palladium, or a nucleophilic substitution reaction under acidic condition; wherein:
   the metal palladium catalyst is selected from palladium acetate, tetrakis(triphenylphosphine) palladium, bistriphenylphosphine palladium dichloride, [1,1′-bis(diphenylphosphino)ferrocene] palladium dichloride or tris(dibenzylideneacetone) dipalladium;
   the alkaline condition refers to a condition in which any of the following substances exists: triethylamine, diisopropylethylamine, pyridine, sodium bicarbonate, sodium carbonate, potassium carbonate, cesium carbonate, lithium hydroxide, sodium hydroxide, potassium hydroxide, sodium hydride, or potassium hydride;
   the acidic condition refers to a condition in which any of the following substances exists:
      acetic acid, trifluoroacetic acid, hydrochloric acid, methanesulfonic acid, p-toluene sulfonic acid, or camphorsulfonic acid;
      see above for the definitions of R¹, R², R³, R⁴ and R⁵.

In another aspect, the present invention provides a pharmaceutical composition comprising the compound according to any one of the above embodiments, a stereoisomer thereof, a prodrug thereof, or a pharmaceutically acceptable salt thereof or a pharmaceutically acceptable solvate thereof, and optionally a pharmaceutically acceptable carrier, diluent or excipient.

In some embodiments, the pharmaceutical composition further comprises a EGFR monoclonal antibody.

In some embodiments, the EGFR monoclonal antibody is Cetuximab or biosimilars thereof.

In another aspect, the present invention further provides use of the compound according to any one of the above embodiments, a stereoisomer thereof, a prodrug thereof, or a pharmaceutically acceptable salt thereof or a pharmaceutically acceptable solvate thereof, and a pharmaceutical composition comprising the compound in the manufacture of a medicament for preventing and/or treating EGFR kinase-mediated cancers and other diseases, especially in the manufacture of a medicament for preventing and/or treating lung cancer (preferably non-small cell lung cancer), particularly in the manufacture of a medicament for preventing and/or treating mutation-type lung cancer (preferably non-small cell lung cancer) with 19Del, L858R, T790M, or C797S mutation or a combination thereof of EGFR kinase, and most preferably in the manufacture of a medicament for preventing and/or treating lung cancer (preferably non-small cell lung cancer) with 19Del single mutation, L858R single mutation, 19Del/T790M dual mutation, L858R/T790M dual mutation, 19Del/T790M/C797S triple mutation or L858R/T790M/C797S triple mutation of EGFR kinase.

In another aspect, the present invention further provides a method of preventing and/or treating EGFR kinase-mediated cancers and other diseases, comprising administering a preventively effective amount and/or a therapeutically effective amount of the above compound or a stereoisomer thereof, a prodrug thereof, or a pharmaceutically acceptable salt thereof or a pharmaceutically acceptable solvate thereof, or the above pharmaceutical composition to a subject in need.

In some embodiments, the method is for preventing and/or treating lung cancer (preferably non-small cell lung cancer).

In some embodiments, the method is for preventing and/or treating mutation-type lung cancer (preferably non-small cell lung cancer) with 19Del, L858R, T790M, or C797S mutation or a combination thereof of EGFR kinase.

In some embodiments, the method is for preventing and/or treating lung cancer (preferably non-small cell lung cancer) with 19Del single mutation, L858R single mutation, 19Del/T790M dual mutation, L858R/T790M dual mutation, 19Del/T790M/C797S triple mutation or L858R/T790M/C797S triple mutation of EGFR kinase.

In another aspect, the present invention further provides the above compound or a stereoisomer thereof, a prodrug thereof, or a pharmaceutically acceptable salt thereof or a pharmaceutically acceptable solvate thereof, or the above pharmaceutical composition, for use in preventing and/or treating EGFR kinase-mediated cancers and other diseases, preferably for use in preventing and/or treating lung cancer (preferably non-small cell lung cancer), more preferably for use in preventing and/or treating mutation-type lung cancer (preferably non-small cell lung cancer) with 19Del, L858R, T790M, or C797S mutation or a combination thereof of EGFR kinase, and most preferably for use in preventing and/or treating lung cancer (preferably non-small cell lung cancer) with 19Del single mutation, L858R single mutation, 19Del/T790M dual mutation, L858R/T790M dual mutation, 19Del/T790M/C797S triple mutation or L858R/T790M/C797S triple mutation of EGFR kinase.

In the present invention, the term “subject” refers to a vertebrate. In some embodiments, the vertebrate refers to a mammal, and the mammal includes, but are not limited to, livestock (such as a cow), pets (such as cat, dog and horse), quadrumana, mouse and rat. In some embodiment, the mammal refers to a human.

In the present invention, the term “effective amount” refers to an amount that can achieve desired treating or preventing effects at necessary doses and time. The “therapeutically effective amount” of the material/molecule of the present invention will vary according to the disease states, ages, sexual distinction and body weights of individuals and the abilities of the material/molecule for initiating desired responses in the individuals. The therapeutically effective amount further covers an amount in which the material/molecule can achieve better beneficial therapeutic effects than any toxic or harmful aftereffects. The “preventively effective amount” refers to an amount that can achieve desired preventive effects at necessary doses and time. Typically but not certainly, since a preventively effective amount is administrated to a subject before disease attacks or at the early stage of diseases, the amount will be lower than the therapeutically effective amount. In the case of cancers, a therapeutically effective amount of a medicine will reduce the number of cancer cells; reduce the volume of tumor; suppress (i.e., retard to an extent, preferably to stop) the infiltration of cancer cells into ambient organs, suppress (i.e., retard to an extent, preferably to stop) tumor metastasis; suppress tumor growths to an extent; and/or relieve one or more symptoms related to cancers to an extent.

### EXPERIMENTAL SECTION

Regarding the examples described below, the compounds of the present invention are synthesized using the methods described herein or other methods well known in the art.

### GENERAL METHODS OF PURIFICATION AND ANALYSIS

Thin layer chromatography was carried out on a silica gel GF254 precoated plate (Qingdao Marine Chemical Plant). Column chromatography was carried out by silica gel (300-400 mesh, Yantai Zhifu Huangwu Silica Gel Development Test Factory) under medium pressure or by a pre-packed silica gel cartridge (ISCO or Welch) with the use of an ISCO Combiflash Rf200 rapid purification system. The ingredient was visualized by UV light λ: 254 nm) or iodine vapor. When necessary, the compound was purified by preparative HPLC through a Waters Symmetry C18 (19 x 50 mm, 5 µm) column or a Waters X Terra RP 18 (30 x 150 mm, 5 µm) column, and was detected by using of a Waters preparative HPLC 600 equipped with a 996 Waters PDA detector and Micromass mod. ZMD single quadrupole mass spectrometry (electrospray ionization, cationic mode). Method 1: Phase A: 0.1% TFA/MeOH 95/5; Phase B: MeOH/H₂O 95/5. Gradient: proceeding at 10 to 90% B for 8 min, keeping at 90% B for 2 min; flow rate: 20 mL/min. Method 2: Phase A: 0.05% NH₄OH/MeOH 95/5; Phase B: MeOH/H₂O 95/5. Gradient: proceeding at 10 to 100% B for 8 min, keeping at 100% B for 2 min; flow rate: 20 mL/min.

¹H-NMR spectra were recorded via a Bruker Avance 600 spectrometer at 600 MHz. Chemical shift (δ) was reported in parts per million (ppm) and coupling constant (J) was measured in Hz. The tetramethylsilane signal was used as a reference (δ= 0 ppm). The following abbreviations were used for peak splitting: s = singlet; br. s. = broad signal; d = doublet; t = triplet; m = multiplet; dd = doublet of doublets.

Electrospray (ESI) mass spectra were obtained via Finnigan LCQ ion trap.

Unless otherwise indicated, all final compounds were homogeneous (with purity not less than 95%), as determined by high performance liquid chromatography (HPLC). HPLC-UV-MS analysis for evaluation of compound purity was performed by combining an ion trap MS device and an HPLC system SSP4000 (Thermo Separation Products) equipped with an autosampler LC Pal (CTC Analytics) and a UV6000LP diode array detector (UV detection 215-400 nm). Device control, data acquisition and processing were performed with Xcalibur 1.2 software (Finnigan). HPLC chromatography was carried out at room temperature and a flow rate of 1 mL/min using a Waters X Terra RP 18 column (4.6 x 50 mm; 3.5 µm). Mobile phase A was ammonium acetate 5 mM buffer (pH 5.5 obtained with acetic acid): acetonitrile 90:10, mobile phase B was ammonium acetate 5 mM buffer (pH 5.5 obtained with acetic acid): acetonitrile 10:90; proceeding at a gradient of 0 to 100% B for 7 min and then keeping at 100% B for 2 min before rebalancing.

Reagent purification was carried out in accordance with the book Purification of Laboratory Chemicals (Perrin, D. D., Armarego, W. L. F. and Perrins Eds, D. R.; Pergamon Press: Oxford, 1980). Petroleum ether was 60-90° C. fraction, ethyl acetate, methanol and dichloromethane were all analytically pure.

### MODE OF CARRYING OUT THE INVENTION

The embodiments of the present invention are described in detail below by way of specific examples, but in any case they cannot be construed as limiting the present invention.

Compounds may be divided into the following three classes:

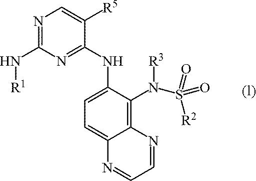

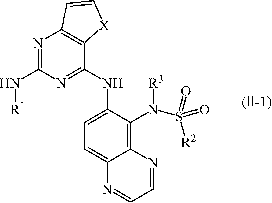

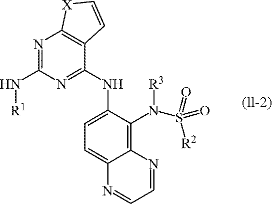

wherein, R¹, R², R³, R⁵ are as above described, X = NH, O or S.

Some of the raw materials and intermediates involved in the synthesis process are described below:

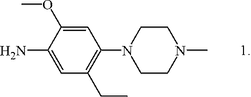 [SMILES: CCc1cc(N)c(OC)cc1N1CCN(C)CC1]

was obtained by the following steps: reducing 2-fluoro-4-hydroxyacetophenone (cas: 98619-07-9, Bidepharm, Shanghai) with palladium carbon and hydrogen gas to obtain 3-fluoro-4-ethylphenol, nitrifying the above reduction product to obtain 3-fluoro-4-ethyl-6-nitrophenol, reacting the above nitrification product with iodomethane (cas: 74-88-4, Xiya Reagent, Shandong) to obtain 3-fluoro-4-ethyl-6-nitroanisole, then reacting the above reaction product with N-methylpiperazine (cas: 109-01-3, Energy, Shanghai) to obtain 3-(N-methylpiperazinyl)-4-ethyl-6-nitroanisole, and finally, reducing the nitro.

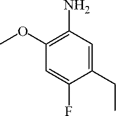

was obtained by reducing the nitro in 3-fluoro-4-ethyl-6-nitroanisole.

The following intermediates were obtained in a similar manner:

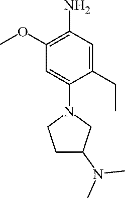

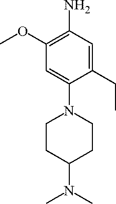 [SMILES: CCc1cc(N)c(OC)cc1N1CCC(N(C)C)CC1]

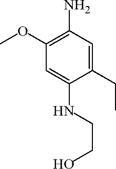

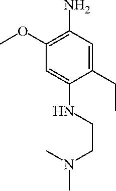 [SMILES: CCc1cc(N)c(OC)cc1NCCN(C)C]

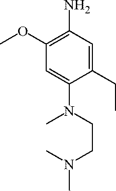 [SMILES: CCc1cc(N)c(OC)cc1N(C)CCN(C)C]

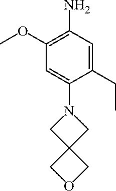 [SMILES: CCc1cc(N)c(OC)cc1N1CC2(COC2)C1]

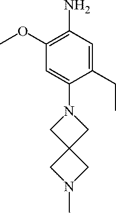 [SMILES: CCc1cc(N)c(OC)cc1N1CC2(CN(C)C2)C1]

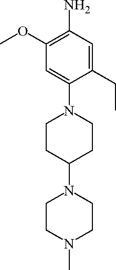 [SMILES: CCc1cc(N)c(OC)cc1N1CCC(N2CCN(C)CC2)CC1]

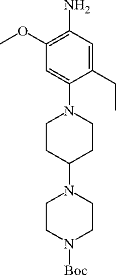 [SMILES: CCc1cc(N)c(OC)cc1N1CCC(N2CCN(C(=O)OC(C)(C)C)CC2)CC1]

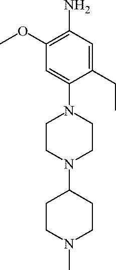 [SMILES: CCc1cc(N)c(OC)cc1N1CCN(C2CCN(C)CC2)CC1]

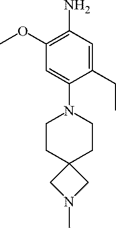 [SMILES: CCc1cc(N)c(OC)cc1N1CCC2(CC1)CN(C)C2]

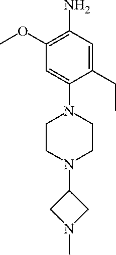 [SMILES: CCc1cc(N)c(OC)cc1N1CCN(C2CN(C)C2)CC1]

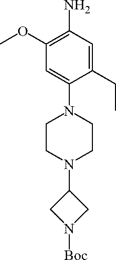 [SMILES: CCc1cc(N)c(OC)cc1N1CCN(C2CN(C(=O)OC(C)(C)C)C2)CC1]

The involved raw materials were: 3-N,N-dimethylaminopyrrolidine (cas: 64021-83-6, TCI, Shanghai), 4-dimethylaminopiperidine (cas: 50533-97-6, Accela ChemBio, Shanghai), 1-methyl-4-(4-piperidyl)piperazine (cas: 53617-36-0, Aikonchem, Jiangsu), ethanolamine (cas: 141-43-5, Aladdin, Shanghai), N,N-dimethylethylenediamine (cas: 108-00-9, Bidepharm, Shanghai), N,N,N′-trimethylethylenediamine (cas: 142-25-6, Bidepharm, Shanghai), 2-oxa-6-azaspiro[3.3]heptane (cas: 174-78-7, Bidepharm, Shanghai), 2-methyl-2,6-diazaspiro[3.3]heptane (cas: 1203567-11-6, Bidepharm, Shanghai), t-butyl 4-(piperidin-4-yl)piperazinyl-1-formate (cas: 205059-24-1, Bidepharm, Shanghai), 1-(1-methylpiperidin-4-yl)piperazine (cas: 23995-88-2, J&K, Shanghai), 2-methyl-2,7-diazaspiro[3.5]nonane dihydrochloride (cas: 1610028-42-6, Bidepharm, Shanghai), 1-(1-methylazetidin-3-yl)piperazine(cas: 864350-81-2, J&K, Shanghai), 1-t-butoxycarbonyl-3-(1-piperazinyl)azetidine (cas: 178311-48-3, Bidepharm, Shanghai).

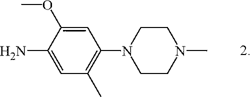

was obtained by reacting 1-fluoro-5-methoxy-2-methyl-4-nitrobenzene (cas: 314298-13-0, Bidepharm, Shanghai) with N-methylpiperazine (cas: 109-01-3, Energy, Shanghai) to obtain 1-(N-methylpiperazinyl)-5-methoxy-2-methyl-4-nitrobenzene, and then reducing the nitro. The following intermediates were obtained in a similar manner:

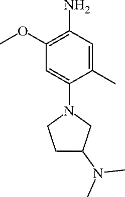

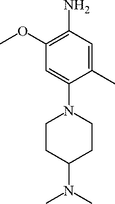

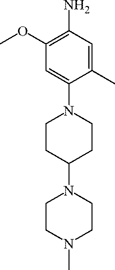

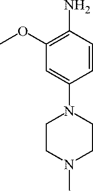 [SMILES: COc1cc(N2CCN(C)CC2)ccc1N]

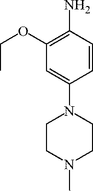 [SMILES: CCOc1cc(N2CCN(C)CC2)ccc1N]

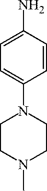

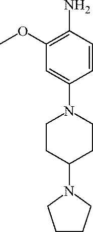 [SMILES: COc1cc(N2CCC(N3CCCC3)CC2)ccc1N]

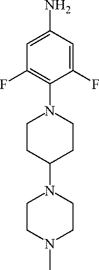 [SMILES: CN1CCN(C2CCN(c3c(F)cc(N)cc3F)CC2)CC1]

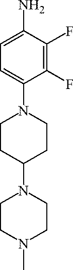 [SMILES: CN1CCN(C2CCN(c3ccc(N)c(F)c3F)CC2)CC1]

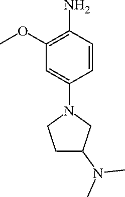

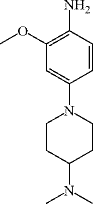

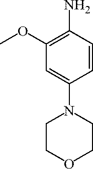

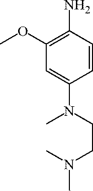 [SMILES: COc1cc(N(C)CCN(C)C)ccc1N]

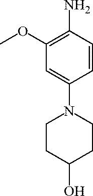 [SMILES: COc1cc(N2CCC(O)CC2)ccc1N]

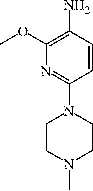 [SMILES: COc1nc(N2CCN(C)CC2)ccc1N]

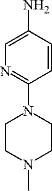

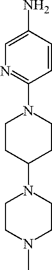 [SMILES: CN1CCN(C2CCN(c3ccc(N)cn3)CC2)CC1]

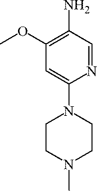 [SMILES: COc1cc(N2CCN(C)CC2)ncc1N]

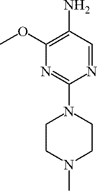 [SMILES: COc1nc(N2CCN(C)CC2)ncc1N]

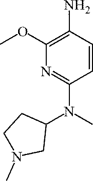

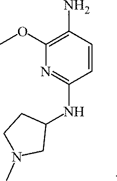

The involved raw materials were: 3-N,N-dimethylaminopyrrolidine (cas: 64021-83-6, TCI, Shanghai), 4-dimethylaminopiperidine (cas: 50533-97-6, Accela ChemBio, Shanghai), 1-methyl-4-(4-piperidyl) piperazine (cas: 53617-36-0, Aikonchem, Jiangsu), 5-fluoro-2-nitroanisole (cas: 448-19-1, Bidepharm, Shanghai), 2-ethoxy-4-fluoro-1-nitrobenzene (cas: 28987-44-2, Bidepharm, Shanghai), p-fluoronitrobenzene (cas: 350-46-9, Bidepharm, Shanghai), 4-(1-pyrrolidyl)piperidine (cas: 5004-07-9, Accela ChemBio, Shanghai), morpholine (cas: 110-91-8, Energy, Shanghai), N,N,N′-trimethylethylenediamine (cas: 142-25-6, Energy, Shanghai), 4-hydroxypiperidine (cas: 5382-16-1, Energy, Shanghai), 1-methyl-3-aminopyrrolidine (cas: 13220-27-4, Shuya, Shanghai), N,N′-dimethyl-3-aminopyrrolidine (cas: 64021-83-6, TCI, Shanghai), 2-methoxy-3-nitro-6-chloropyridine (cas: 40851-91-0, Bidepharm, Shanghai), 2-fluoro-5-nitropyridine (cas: 456-24-6, Bidepharm, Shanghai), 2-chloro-4-methoxy-5-nitropyridine (cas: 607373-83-1, Bidepharm, Shanghai), 2-chloro-4-methoxy-5-nitropyrimidine (cas: 282102-07-2, Bidepharm, Shanghai), 3,4,5-trifluoronitrobenzene (cas: 66684-58-0, Bidepharm, Shanghai), 2,3,4-trifluoronitrobenzene (cas: 771-69-7, Bidepharm, Shanghai).

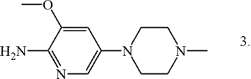

was obtained by the steps of nitrifying 3-fluoro-5-hydroxypyridine (cas: 209328-55-2, Bidepharm, Shanghai) to obtain 3-fluoro-5-hydroxy-6-nitropyridine, then reacting the above nitrification product with iodomethane (cas: 74-88-4, Xiya Reagent, Shandong) to obtain 3-fluoro-5-methoxy-6-nitropyridine, reacting the above reaction product with N-methylpiperazine (cas: 109-01-3, Energy, Shanghai) to obtain and then reducing the nitro.

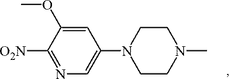

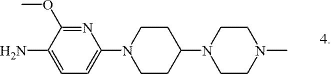

was obtained by the steps of reacting 2,6-dichloro-3-nitropyridine with sodium methoxide (cas: 124-41-4, Energy, Shanghai) to obtain 2-methoxy-3-nitro-6-chloropyridine, reacting the above reaction product with 1-methyl-4-(4-piperidyl)piperazine (cas: 53617-36-0, Aikonchem, Jiangsu) to obtain

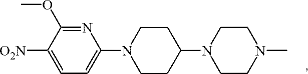

and further reducing the amino.

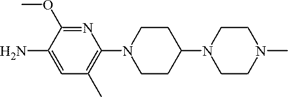

was obtained from 2,6-dichloro-3-nitro-5-methylpyridine according to a similar method.

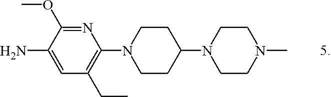

was obtained by the following steps: reacting

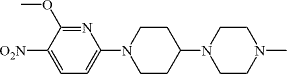

with liquid bromine (cas: 7726-95-6, Sinopharm, Shanghai) to obtain

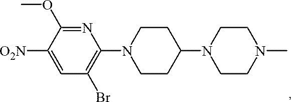

then reacting the above reaction product with pinacol vinylboronate (cas: 75927-49-0, Bidepharm, Shanghai) to obtain

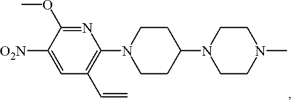

and further reducing the nitro and the double bonds.

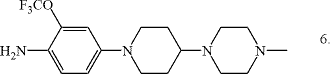 [SMILES: CN1CCN(C2CCN(c3ccc(N)c(OC(F)(F)F)c3)CC2)CC1]

was obtained by the following steps: nitrifying 1-bromo-2-fluoro-4-trifluoromethoxybenzene (cas: 168971-68-4, Bidepharm, Shanghai) to obtain 2-trifluoromethoxy-4-fluoro-5-bromonitrobenzene, then reacting the above nitrification product with 1-methyl-4-(4-piperidyl)piperazine (cas: 53617-36-0, Aikonchem, Jiangsu) to obtain

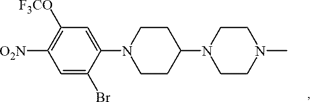

and further reducing the nitro and debrominating.

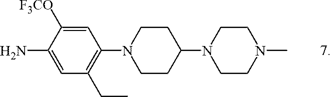 [SMILES: CCc1cc(N)c(OC(F)(F)F)cc1N1CCC(N2CCN(C)CC2)CC1]

was obtained by reacting

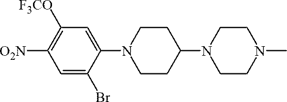 [SMILES: CN1CCN(C2CCN(c3cc(OC(F)(F)F)c([N+](=O)[O-])cc3Br)CC2)CC1]

with pinacol vinylboronate (cas: 75927-49-0, Bideharm, Shanghai) to obtain

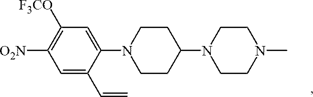

and then reducing the nitro and the double bonds.

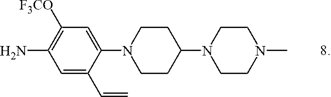 [SMILES: C=Cc1cc(N)c(OC(F)(F)F)cc1N1CCC(N2CCN(C)CC2)CC1]

was obtained by reducing the nitro in

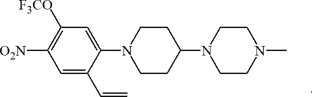

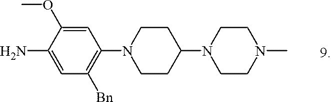 [SMILES: COc1cc(N2CCC(N3CCN(C)CC3)CC2)c(Cc2ccccc2)cc1N]

was obtained by the following steps: nitrifying 2-fluoro-4-methoxybenzaldehyde (cas: 331-64-6) to obtain 2-fluoro-4-methoxy-5-nitrobenzaldehyde, reducing the above nitrification product to obtain 2-fluoro-4-methoxy-5-nitrobenzyl alcohol, further bromating the above reduced product to obtain 2-fluoro-4-methoxy-5-nitrobenzyl bromide, reacting the above bromation product with phenylboronic acid (cas: 98-80-6, Energy, Shanghai) to obtain 1-benzyl-2-fluoro-4-methoxy-5-nitrobenzene, reacting the above reaction product with 1-methyl-4-(4-piperidyl)piperazine (cas: 53617-36-0, Aikonchem, Jiangsu) to obtain

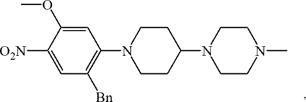

and then reducing the nitro. The phenylboronic acid was replaced with cyclopropylboronic acid (cas: 411235-57-9, Bidepharm, Shanghai), and

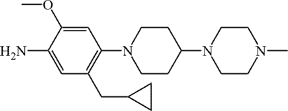 [SMILES: COc1cc(N2CCC(N3CCN(C)CC3)CC2)c(CC2CC2)cc1N]

was obtained according to a similar method.

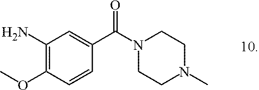

was obtained by reacting 4-methoxy-3-nitrobenzoic acid (cas: 89-41-8, Bidepharm, Shanghai) with N-methylpiperazine (cas: 109-01-3, Energy, Shanghai) to obtain

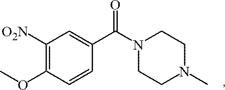

and then reducing the nitro; the following intermediates were obtained in a similar manner:

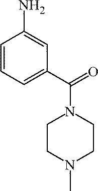 [SMILES: CN1CCN(C(=O)c2cccc(N)c2)CC1]

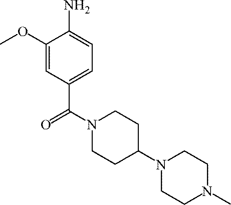

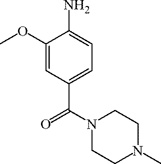

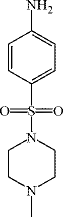 [SMILES: CN1CCN(S(=O)(=O)c2ccc(N)cc2)CC1]

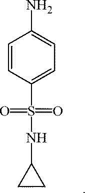

The involved raw materials were: 3-methoxy-4-nitrobenzoic acid (cas: 5081-36-7, Bidepharm, Shanghai), p-nitrobenzenesulfonyl chloride (cas: 98-74-8, Aladdin, Shanghai), m-nitrobenzoic acid (cas: 121-92-6, Energy, Shanghai), 1-methyl-4-(4-piperidyl)piperazine (cas: 53617-36-0, Aikonchem, Jiangsu), N-methylpiperazine (cas: 109-01-3, Energy, Shanghai), cyclopropylamine (cas: 765-30-0, Energy, Shanghai).

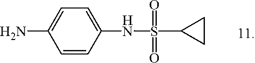 [SMILES: Nc1ccc(NS(=O)(=O)C2CC2)cc1]

was obtained by reacting 4-t-butoxycarbonylaminoaniline (cas: 71026-66-9, Bidepharm, Shanghai) with cyclopropylsulfonyl chloride (cas: 139631-62-2, Energy, Shanghai) to obtain

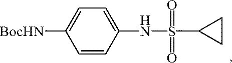

and then removing t-butoxycarbonyl.

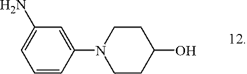 [SMILES: Nc1cccc(N2CCC(O)CC2)c1]

was obtained by reacting m-bromonitrobenzene (cas: 585-79-5, Energy, Shanghai) with 4-hydroxypiperidine (cas: 5382-16-1, Energy, Shanghai) to obtain

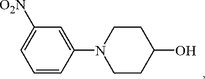

and then reducing the nitro; the following intermediates were obtained in a similar manner:

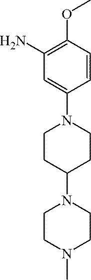 [SMILES: COc1ccc(N2CCC(N3CCN(C)CC3)CC2)cc1N]

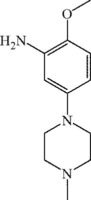 [SMILES: COc1ccc(N2CCN(C)CC2)cc1N]

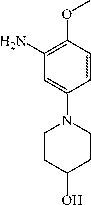 [SMILES: COc1ccc(N2CCC(O)CC2)cc1N]

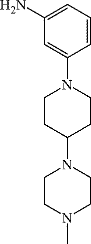 [SMILES: CN1CCN(C2CCN(c3cccc(N)c3)CC2)CC1]

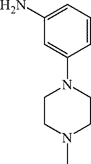

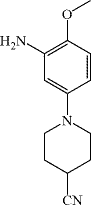 [SMILES: COc1ccc(N2CCC(C#N)CC2)cc1N]

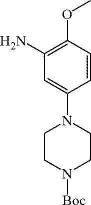 [SMILES: COc1ccc(N2CCN(C(=O)OC(C)(C)C)CC2)cc1N]

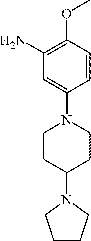 [SMILES: COc1ccc(N2CCC(N3CCCC3)CC2)cc1N]

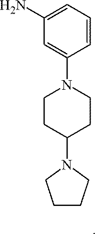

The involved raw materials were: 4-bromo-2-nitroanisole (cas: 33696-00-3, Shuya, Shanghai), 4-hydroxypiperidine (cas: 5382-16-1, Energy, Shanghai), 1-methyl-4-(4-piperidyl)piperazine (cas: 53617-36-0, Aikonchem, Jiangsu), N-methylpiperazine (cas: 109-01-3, Energy, Shanghai), 4-cyanopiperidine (cas: 4395-98-6, J&K, Shanghai), 1-t-butoxycarbonylpiperazine (cas: 57260-71-6, Bidepharm, Shanghai), 4-(pyrrolidin-1-yl)piperidine (cas: 5004-07-9, Bidepharm, Shanghai).

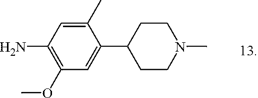 [SMILES: COc1cc(C2CCN(C)CC2)c(C)cc1N]

was obtained by the following steps: reacting 2-nitro-4-methyl-5-chlorophenol (cas: 100278-74-8, Bidepharm, Shanghai) with iodomethane (cas: 74-88-4, Xiya Reagent, Shandong) to obtain 2-nitro-4-methyl-5-chloroanisole, reacting the above reaction product with pyridine-4-boronic acid (cas: 1692-15-5, Shuya, Shanghai) to obtain

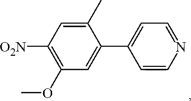

which was further reacted with iodomethane (cas: 74-88-4, Xiya Reagent, Shandong) to obtain

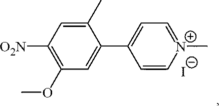

and finally reducing the nitro and the pyridine ring; the iodomethane was replaced with bromated isopropane (cas: 75-26-3, Macklin, Shanghai), and

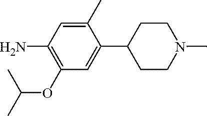 [SMILES: Cc1cc(N)c(OC(C)C)cc1C1CCN(C)CC1]

may be obtained according to a similar method.

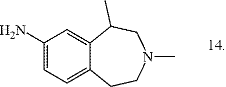

was obtained by the following steps: reacting p-nitrophenylethanol (cas: 100-27-6, Bidepharm, Shanghai) with 1,3-dibromo-5,5-dimethylhydantoin (cas: 77-48-5, Bidepharm, Shanghai) to obtain 2-bromo-4-nitrophenylethanol, reacting the above reaction product with methylsulfonyl chloride (cas: 124-63-0, Xiya Reagent, Shandong) to obtain 2-bromo-4-nitrophenylethyl mesylate, which was continuously reacted with methylallylamine (cas: 627-37-2, Energy,Shanghai) to obtain

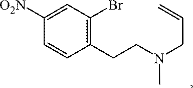

further converting to

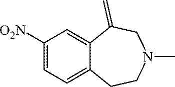 [SMILES: C=C1CN(C)CCc2ccc([N+](=O)[O-])cc21]

via a Heck reaction, and finally reducing the nitro and the double bonds.

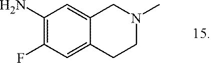

was obtained by the following steps: reacting 6-fluoro-3,4-dihydro-2H-isoquinolin-1-one (cas: 214045-84-8, Bidepharm, Shanghai) with iodomethane (cas: 74-88-4, Xiya Reagent, Shandong) to obtain 2-methyl-6-fluoro-3,4-dihydro-2H-isoquinolin-1-one, reacting the above reaction product with fuming nitric acid (cas: 7697-37-2, Hushi, Shanghai) to obtain 2-methyl-6-fluoro-7-nitro-3,4-dihydro-2H-isoquinolin-1-one, further reducing the carbonyl to obtain 2-methyl-6-fluoro-7-nitro-1,2,3,4-tetrahydroisoquinoline, and finally reducing the nitro;

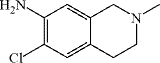

was obtained from 6-chloro-3,4-dihydro-2H-isoquinolin-1-one (cas: 22246-02-2, Bidepharm, Shanghai) according to a similar method;

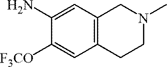 [SMILES: CN1CCc2cc(OC(F)(F)F)c(N)cc2C1]

was obtained by the steps of reducing 3-(trifluoromethoxy)phenylacetonitrile (cas: 108307-56-8, Bidepharm, Shanghai) to obtain 2-(3-trifluoromethoxyphenyl)ethylamine, reacting the above reduction product with ethyl chloroformate (cas: 541-41-3, Xiya Reagent, Shandong) to obtain 2-(3-trifluoromethoxyphenyl)ethylaminoformate, which was further converted into 6-trifluoromethoxy-3,4-dihydro-2H-isoquinolin-1-one under the action of trifluoromethanesulfonic anhydride (cas: 358-23-6, Energy, Shanghai), and then performing a similar method;

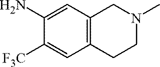 [SMILES: CN1CCc2cc(C(F)(F)F)c(N)cc2C1]

was obtained from m-trifluoromethylphenylacetonitrile (cas: 2338-76-3, Bidepharm, Shanghai) according to a similar method; 2-methyl-6-fluoro-7-nitro-3,4-dihydro-2H-isoquinolin-1-one was reacted with methanol (cas: 67-56-1, Hushi, Shanghai) to obtain 2-methyl-6-methoxy-7-nitro-3,4-dihydro-2H-isoquinolin-1 -one, and then

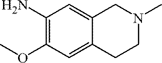

was obtained according to a similar method.

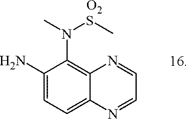

was obtained by the following steps: reacting 6-nitroquinoxaline (cas: 6639-87-8, Bidepharm, Shanghai), and hydroxyamine hydrochloride (cas: 5470-11-1, Energy, Shanghai) to obtain 5-amino-6-nitro quinoxaline, then converting the above reaction product to 5-chloro-6-nitroquinoxaline, which was further reacted with N-methyl methanesulfonamide (cas: 1184-85-6, Bidepharm, Shanghai) to obtain

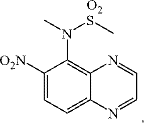

and finally reducing the nitro. The following intermediates were obtained in a similar manner:

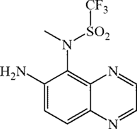 [SMILES: CN(c1c(N)ccc2nccnc12)S(=O)(=O)C(F)(F)F]

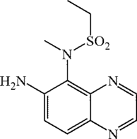 [SMILES: CCS(=O)(=O)N(C)c1c(N)ccc2nccnc12]

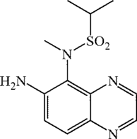 [SMILES: CC(C)S(=O)(=O)N(C)c1c(N)ccc2nccnc12]

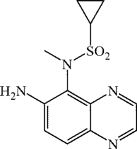 [SMILES: CN(c1c(N)ccc2nccnc12)S(=O)(=O)C1CC1]

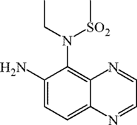 [SMILES: CCN(c1c(N)ccc2nccnc12)S(C)(=O)=O]

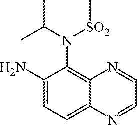 [SMILES: CC(C)N(c1c(N)ccc2nccnc12)S(C)(=O)=O]

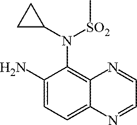 [SMILES: CS(=O)(=O)N(c1c(N)ccc2nccnc12)C1CC1]

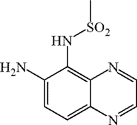 [SMILES: CS(=O)(=O)Nc1c(N)ccc2nccnc12]

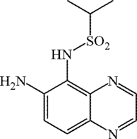 [SMILES: CC(C)S(=O)(=O)Nc1c(N)ccc2nccnc12]

The involved raw materials were: methylamine (cas: 74-89-5, Energy, Shanghai), ethylamine (cas: 75-04-7, Aladdin, Shanghai), isopropylamine (cas: 75-31-0, Energy, Shanghai), cyclopropylamine (cas: 765-30-0, Energy, Shanghai), trifluoromethanesulfonyl chloride (cas: 421-83-0, Aladdin, Shanghai), ethylsulfonyl chloride (cas: 594-44-5, Energy, Shanghai), isopropylsulfonyl chloride (cas: 10147-37-2, TCI, Shanghai), cyclopropylsulfonyl chloride (cas: 139631-62-2, Energy, Shanghai), methylsulfamide (cas: 3144-09-0, Bidepharm, Shanghai), isopropylsulfamide (cas: 81363-76-0, Bidepharm, Shanghai).

17. 1,3-dimethyl-5-aminopyrazole (cas: 3524-32-1, J&K, beijing).

18. 1-methyl-3-aminopyrazole (cas: 1904-31-0, Bidepharm, Shanghai).

19. 1-methyl-4-aminopyrazole (cas: 69843-13-6, Bidepharm, Shanghai).

20. 1-methyl-4-aminoimidazole (cas: 79578-98-6, Bidepharm, Shanghai).

21. 1-methyl-4-aminopiperidine (cas: 41838-46-4, Energy, Shanghai).

22. N,N-dimethylethylenediamine (cas: 108-00-9, Accela ChemBio, Shanghai).

23. ethanolamine (cas: 141-43-5, Aladdin, Shanghai).

24. trans-1,4-diaminocyclohexane (cas: 2615-25-0, Energy, Shanghai).

25.

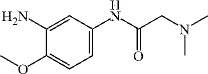

was obtained by the following steps: reacting 3-nitro-4-methoxyaniline (cas: 577-72-0, Bidepharm, Shanghai) with bromoacetyl bromide (cas: 598-21-0, Energy, Shanghai), further reacting the above reaction product with dimethylamine (cas: 124-40-3, Meryer, Shanghai) to obtain

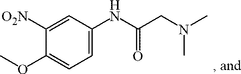

then reducing the nitro.

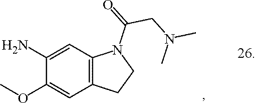

cas: 1116229-84-5, Bidepharm, Shanghai.

27. 3-methylpiperazinyl-1-methylene)-5-trifluoromethylaniline (cas: 853296-94-3, Aikonchem, Jiangsu).

28. 3-trifluoromethyl-4-(4-methylpiperazinyl-1-methylene)-aniline (cas: 694499-26-8, Shuya, Shanghai).

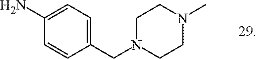

was obtained by reacting p-nitrobenzaldehyde (cas: 555-16-8, Bidepharm, Shanghai) with N-methylpiperazine (cas: 109-01-3, Energy, Shanghai) to obtain

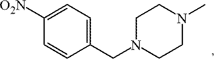

and then reducing the nitro. The following intermediates were obtained in a similar manner:

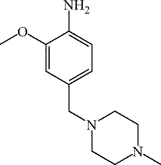

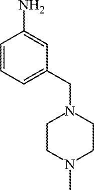

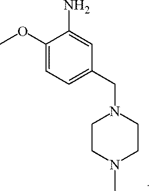

The involved raw materials were: 3-methoxy-4-nitrobenzaldehyde (cas: 80410-57-7, Bidepharm, Shanghai), 4-methoxy-3-nitrobenzaldehyde (cas: 31680-08-7, Bidepharm, Shanghai), m-nitrobenzaldehyde(cas: 99-61-6, Bidepharm, Shanghai), N-methylpiperazine (cas: 109-01-3, Energy, Shanghai).

30. 3-amino-6-methylpyridine (cas: 3430-14-6, Energy, Shanghai).

31. 4-aminopyridine (cas: 504-24-5, Energy, Shanghai).

32. 2,4-dichloro-5-bromopyrimidine (cas: 36082-50-5, Bidepharm, Shanghai).

33. 2,4,5-trichloropyrimidine (cas: 5750-76-5, Energy, Shanghai).

34. 2,4-dichloro-5-fluoropyrimidine (cas: 2927-71-7, Bidepharm, Shanghai).

35. 2,4-dichloro-5-trifluoromethylpyrimidine (cas: 3932-97-6, Bidepharm, Shanghai).

36. 2,4-dichloro-5-cyanopyrimidine (cas: 3177-24-0, Bidepharm, Shanghai).

37. 2,4-dichloro-5-nitropyrimidine (cas: 49845-33-2, Aikonchem, Shanghai).

38. 2,4-dichlorothieno[3,2-d]pyrimidine (cas: 16234-14-3, PharmaBlock, Jiangsu).

39. 2,4-dichlorofuro[3,2-d]pyrimidine (cas: 956034-07-4, Aikonchem, Shanghai).

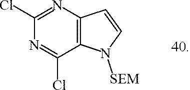 [SMILES: C[Si](C)(C)CCOCn1ccc2nc(Cl)nc(Cl)c21]

was obtained by reacting 2,4-dichloropyrrolo[3,2-d]pyrimidine (cas:63200-54-4, PharmaBlock, Jiangsu) with 2-(trimethylsilyl)ethoxymethyl chloride (CAS: 76513-69-4, Efe, Shanghai).

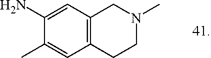

(cas: 1268394-21-3, Aikonchem, Shanghai). 42. 2,4-dichlorothieno[2,3-d]pyrimidine (cas: 18740-39-1, Chemlin, Jiangsu).

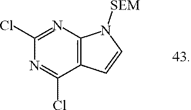 [SMILES: C[Si](C)(C)CCOCn1ccc2c(Cl)nc(Cl)nc21]

was obtained by reacting 2,4-dichloro-7H-pyrrolo[2,3-d]pyrimidine (cas: 90213-66-4, Shuya, Shanghai) with 2-(trimethylsilyl)ethoxymethyl chloride (CAS: 76513-69-4, Efe, Shanghai).

44. 2,4-dichlorofuro[2,3-d]pyrimidine (cas: 1000577-84-3, Bidepharm, Shanghai).

45. 2,4-dichloro-5-cyclopropylpyrimidine (cas: 1190379-86-2, Bidepharm, Shanghai).

46. 2,4-dichloro-5-methylpyrimidine (cas: 1780-31-0, Bidepharm, Shanghai).

47. 2,4-dichloro-5-methoxypyrimidine (cas: 19646-07-2, Bidepharm, Shanghai).

48. 2,4-dichloropyrimidine (cas: 3934-20-1, Bidepharm, Shanghai).

49. 2,4-dichloro-5-ethylpyrimidine (cas: 34171-40-9, Bidepharm, Shanghai).

50. 2,4-dichloro-5-isopropylpyrimidine (cas: 514843-12-0, Bidepharm, Shanghai).

51. 2,4-dichloro-5-ethoxypyrimidine (cas: 280582-25-4, Bidepharm, Shanghai).

52. 1-ethyl-3-aminopyrazole (cas: 55361-49-4, Bidepharm, Shanghai).

53. 1-isopropyl-3-aminopyrazole (cas: 857267-04-0, Bidepharm, Shanghai).

54. 1,5-dimethyl-3-aminopyrazole (cas: 35100-92-6, Bidepharm, Shanghai).

55. 1-ethyl-4-aminopyrazole (cas: 876343-24-7, Bidepharm, Shanghai).

56. 1-isopropyl-4-aminopyrazole (cas: 97421-16-4, Bidepharm, Shanghai).

57. 1-ethyl-3-methyl-4-aminopyrazole (cas: 947763-34-0, Bidepharm, Shanghai).

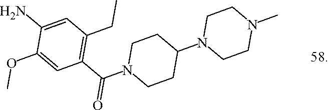

was obtained by the following steps: oxidizing 1-bromo-2-methyl-4-methoxy-5-nitrobenzene (cas: 89978-56-3, Jiuding chem, Shanghai) into 2-bromo-4-nitro-5-methoxybenzoic acid, which was further suffered from condensation reaction with 1-methyl-4-(4-piperidyl)piperazine (cas: 53617-36-0,Aikonchem, Jiangsu) to obtain

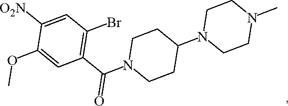

coupling the above reaction product with potassium vinyltrifluoroborate (cas: 13682-77-4, Bidepharm, Shanghai) to obtain

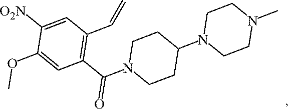

and performing a reduction.

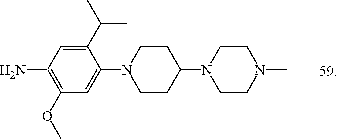 [SMILES: COc1cc(N2CCC(N3CCN(C)CC3)CC2)c(C(C)C)cc1N]

was obtained by the following steps: reacting 1-bromo-2-fluoro-4-methoxy-5-nitrobenzene (cas: 1352244-77-9, Bidepharm, Shanghai) with 1-methyl-4-(4-piperidyl)piperazine (cas: 53617-36-0, Aikonchem, Jiangsu) to obtain

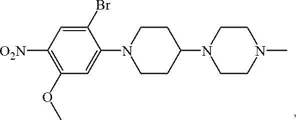

coupling the above reaction product with isopropenylboronic acid pinacol ester (cas: 126726-62-3, Bidepharm, Shanghai) to obtain

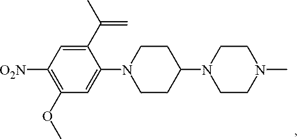

and further performing a reduction. The following intermediates were obtained in a similar manner:

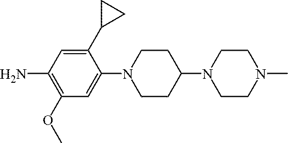 [SMILES: COc1cc(N2CCC(N3CCN(C)CC3)CC2)c(C2CC2)cc1N]

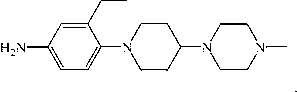

The involved raw materials were: 3-bromo-4-fluoronitrobenzene (cas: 701-45-1, Bidepharm, Shanghai), cyclopropylboronic acid pinacol ester (cas: 126689-01-8, Bidepharm, Shanghai), vinylboronic acid pinacol ester (cas: 75927-49-0, Bidepharm, Shanghai).

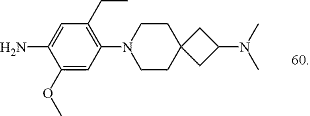 [SMILES: CCc1cc(N)c(OC)cc1N1CCC2(CC1)CC(N(C)C)C2]

was obtained by the following steps: reacting tert-butyl 2-oxo-7-azaspiro[3.5]nonane-7-carboxylate (cas: 203661-69-2, Bidepharm, Shanghai) with dimethylamine (cas: 124-40-3, Aladdin, Shanghai) to obtain

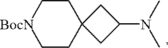

after removing t-butoxycarbonyl, reacting the above reaction product with 3-fluoro-4-ethyl-6-nitroanisole in (1) to obtain

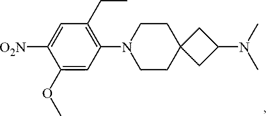

and thenreducing the nitro.

The embodiments of the present invention are described in detail below by way of specific examples, but in any case they cannot be construed as limiting the present invention.

### Synthesis of Compound 1-1

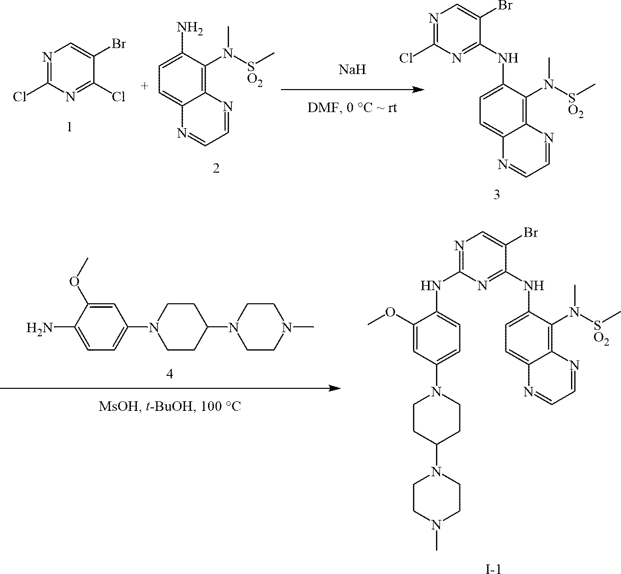

First step: Compound 2 (504 mg, 2 mmol) was dissolved in DMF (5 mL), and, sodium hydride (60% concentration, dispersed in liquid paraffin wax, 160 mg, 4 mmol) was added thereto in batches at 0° C. After 5 minutes, compound 1 (501 mg, 2.2 mmol) was added and reacted by self-heating for 1 hour, the completion of the reaction was detected by TLC and LCMS, and then 100 mL of water were added to the reaction mixture to precipitate solids. The solids were filtered and dried under reduced pressure to obtain compound 3, which was directly used for the next step reaction.

Second step: Compound 3 (44 mg, 0.1 mmol), compound 4 (30.4 mg, 0.1 mmol) and methanesulfonic acid (19 µL, 0.3 mmol) in t-BuOH (2 mL) were heated at 100° C. for 4 h, and the completion of the reaction was detected by TLC and LCMS. After cooling, the reaction mixture was concentrated, and purified by silica gel chromatograph (elution with dichloromethane/methanol) and then further purified by preparative HPLC (aqueous solution containing 0.35% trifluoroacetic acid and methanol as mobile phase), to obtain compound l-1.

### Synthesis of Compound I-1

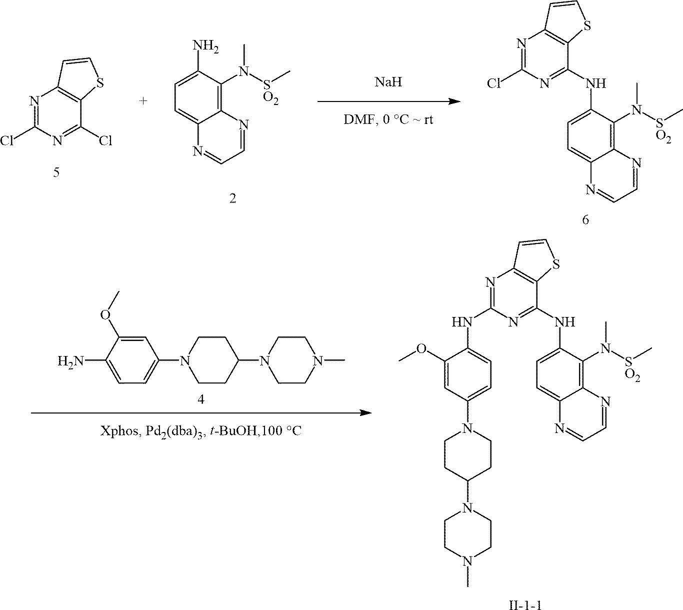

First step: Compound 6 was obtained from compound 5 and compound 2 according to the similar method to that for preparation of compound 3.

Second step: Compound 6 (42.1 mg, 0.1 mmol), compound 4 (30.4 mg, 0.1 mmol), 2-dicyclohexylphosphino-2,4,6-triisopropylbiphenyl (7.2 mg, 0.015 mmol), tri(dibenzylideneacetone) dipalladium (9.2 mg, 0.01 mmol) and potassium carbonate (41.4 mg, 0.3 mmol) were dispersed in t-BuOH (2 mL), and after nitrogen gas replacement, the reaction system solution was heated and stirred by placing in an oil bath preheated to 100° C. And after 6 hours, the completion of the reaction was detected by TLC and LCMS. After cooling, the reaction mixture was concentrated, and purified by silica gel chromatograph (elution with dichloromethane/methanol) and then further purified by preparative HPLC (aqueous solution containing 0.35% trifluoroacetic acid and methanol as mobile phase), to obtain compound ll-1-1.

### Preparation of Compound II-1-18

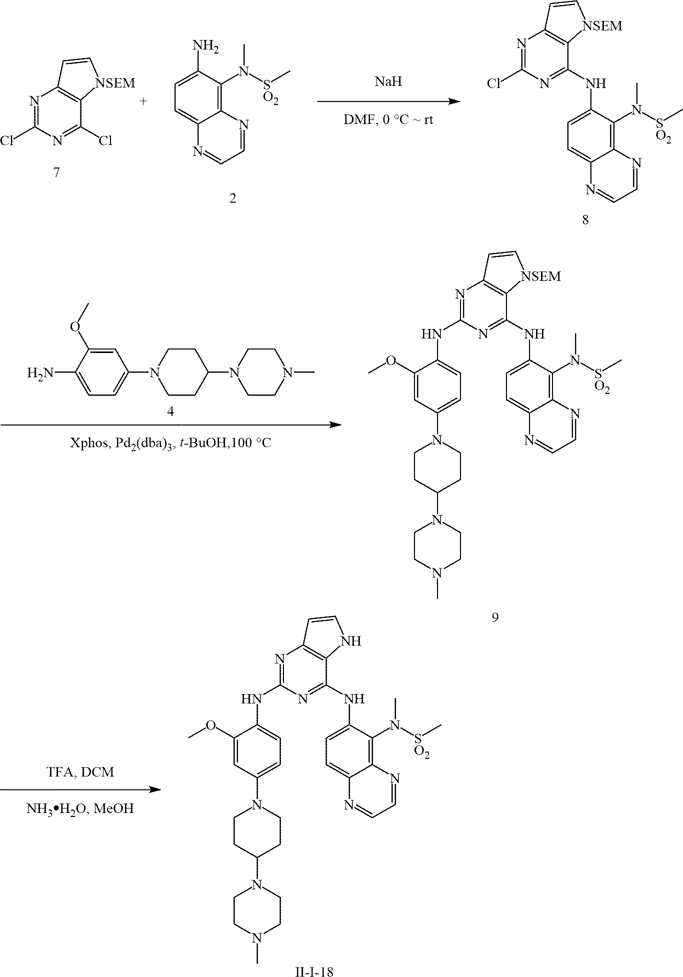

First step: Compound 8 was obtained from compound 7 and compound 2 according to the similar method to that for preparation of compound 3.

Second step: Compound 9 was obtained from compound 8 and compound 4 according to the similar method to that for preparation of compound ll-1-1.

Third step: After compound 9 (30 mg, 0.037 mmol) was stirred for 2 hours in TFA/DCM (⅟2 mL) at room temperature, the completion of the reaction was detected by TLC and LCMS, and the reaction mixture was concentrated. The residues were dissolved in methanol (2 mL), and 25% ammonia (1 mL) was added, and after the reaction continued at room temperature for 4 hours, the completion of the reaction was detected by TLC and LCMS. The reaction mixture was diluted with dichloromethane (50 mL), washed with saturated saline solution (30 mL) once, and separated. The organic phase was dried with anhydrous Na₂SO₄, filtered, concentrated, purified by silica gel chromatograph (elution with dichloromethane/methanol), and then further purified by preparative HPLC (aqueous solution containing 0.35% trifluoroacetic acid and methanol as mobile phase), to obtain compound ll-1-18.

Other compounds could be synthesized by similar methods.

In the following table, it lists the specific compounds and their structural characterization data.

| | | |
|---|---|---|
| No. | Structure | ¹H NMR and/or MS data |
| I-1 | 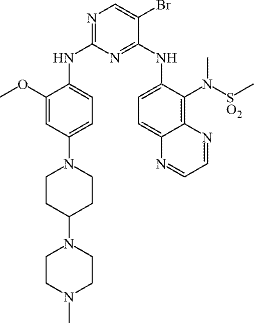 | ¹H NMR (600 MHz, Methanol-d₄) δ 8.97 (d, J = 1.7 Hz, 1H), 8.90 (d, J = 1.7 Hz, 1H), 8.79 (d, J = 9.5 Hz, 1H), 8.28 (s, 1H), 8.10 (d, J = 9.4 Hz, 1H), 7.92 - 7.70 (m, 1H), 7.00 (s, 1H), 6.83 (d, J = 8.9 Hz, 1H), 3.91 (s, 3H), 3.87 - 3.74 (m, 2H), 3.48 (s, 3H), 3.43 - 3.33 (m, 8H), 3.28 - 3.26 (m, 1H), 3.20 (s, 3H), 2.97 - 2.90 (m, 2H), 2.88 (s, 3H), 2.23 - 2.13 (m, 2H), 2.01 - 1.89 (m, 2H). MS (ESI) m/z: 711 [M+H]⁺. |
| I-2 | 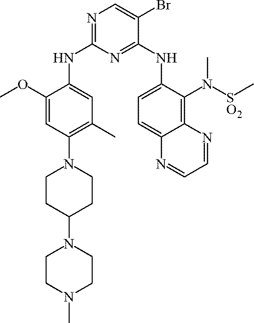 | ¹H NMR (600 MHz, Methanol-d₄) δ 8.99 (d, J = 1.9 Hz, 1H), 8.93 (d, J = 1.9 Hz, 1H), 8.65 - 8.50 (m, 1H), 8.27 (s, 1H), 8.07 (d, J = 9.3 Hz, 1H), 7.36 (s, 1H), 6.83 (s, 1H), 3.85 (s, 3H), 3.70 - 3.48 (m, 8H), 3.47 (s, 3H), 3.36 - 3.33 (m, 2H), 3.28 - 3.23 (m, 1H), 3.18 (s, 3H), 2.96 (s, 3H), 2.95 - 2.87 (m, 2H), 2.26 - 2.19 (m, 2H), 2.05 (s, 3H), 2.01 - 1.90 (m, 2H). MS (ESI) m/z: 725 [M+H]⁺. |
| I-3 | 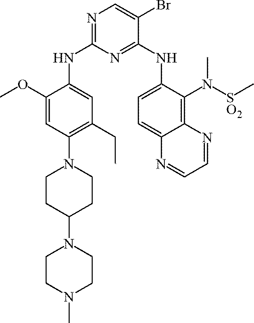 | ¹H NMR (600 MHz, MeOD) δ 8.99 (d, J = 1.8 Hz, 1H), 8.93 (d, J = 1.8 Hz, 1H), 8.60 - 8.42 (m, 1H), 8.28 (s, 1H), 8.11 - 7.97 (m, 1H), 7.39 (s, 1H), 6.85 (s, 1H), 3.85 (s, 3H), 3.72 - 3.53 (m, 8H), 3.47 (s, 3H), 3.37 - 3.34 (m, 1H), 3.26 - 3.21 (m, 2H), 3.17 (s, 3H), 2.97 (s, 3H), 2.95 - 2.88 (m, 2H), 2.56 - 2.36 (m, 2H), 2.34 - 2.18 (m, 2H), 2.13 - 1.89 (m, 2H), 1.04 - 0.73 (m, 3H). MS (ESI) m/z: 739 [M+H]⁺. |
| I-4 | 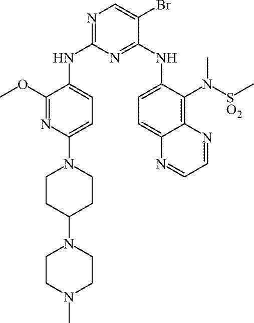 | ¹H NMR (600 MHz, MeOD) δ 8.98 (d, J = 1.9 Hz, 1H), 8.92 (d, J = 1.9 Hz, 1H), 8.80 - 8.45 (m, 1H), 8.35 - 8.18 (m, 1H), 8.16 - 7.97 (m, 1H), 7.60 - 7.47 (m, 1H), 6.50 - 6.23 (m, 1 H), 4.64 - 4.40 (m, 2H), 3.88 (s, 3H), 3.77 - 3.52 (m, 9H), 3.48 (s, 3H), 3.49 - 3.43 (m, 2H), 3.18 (s, 3H), 2.96 (s, 3H), 2.95 - 2.92 (m, 1H), 2.33 - 2.10 (m, 2H), 1.91 - 1.62 (m, 2H). MS (ESI) m/z: 712 [M+H]⁺. |
| I-5 | 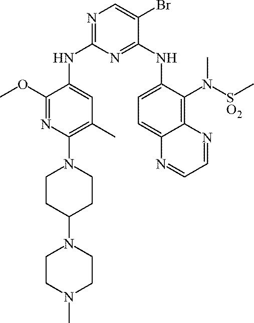 | ¹H NMR (600 MHz, MeOD) δ 8.98 (d, J = 1.8 Hz, 1H), 8.96 - 8.89 (m, 1H), 8.75 - 8.46 (m, 1H), 8.37 - 8.21 (m, 1H), 8.19 - 7.85 (m, 1H), 7.44 (s, 1H), 3.95 (s, 3H), 3.86 - 3.73 (m, 2H), 3.65 - 3.52 (m, 8H), 3.50 (s, 3H), 3.38 - 3.33 (m, 1H), 3.20 (s, 3H), 2.95 (s, 3H), 2.93 - 2.86 (m, 2H), 2.12 - 2.04 (m, 2H), 2.05 - 1.90 (m, 3H), 1.86 - 1.59 (m, 2H). MS (ESI) m/z: 726 [M+H]⁺. |
| I-6 | 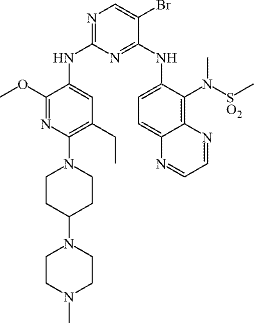 | ¹H NMR (600 MHz, MeOD) δ 8.99 (d, J = 1.8 Hz, 1H), 8.93 (d, J = 1.9 Hz, 1H), 8.47 (s, 1H), 8.34 - 8.27 (m, 1H), 8.03 (d, J = 9.4 Hz, 1H), 7.70 (s, 1H), 3.93 (s, 3H), 3.78 - 3.61 (m, 8H), 3.58 - 3.52 (m, 2H), 3.47 (s, 3H), 3.45 - 3.38 (m, 1H), 3.17 (s, 3H), 3.05 - 3.00 (m, 2H), 2.99 (s, 3H), 2.50 - 2.29 (m, 2H), 2.22 (d, J = 11.8 Hz, 2H), 2.05 - 1.91 (m, 2H), 1.01 - 0.73 (m, 3H). MS (ESI) m/z: 740 [M+H]⁺. |
| I-7 | 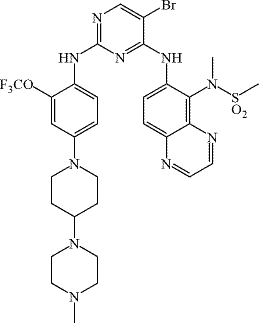 [SMILES: CN1CCN(C2CCN(c3ccc(Nc4ncc(Br)c(Nc5ccc6nccnc6c5N(C)S(C)(=O)=O)n4)c(OC(F)(F)F)c3)CC2)CC1] | ¹H NMR (600 MHz, Methanol-d4) δ 8.94 (d, J = 1.8 Hz, 1H), 8.85 (d, J = 1.9 Hz, 1H), 8.76 (d, J = 9.7 Hz, 1H), 8.24 (s, 1H), 7.93 (d, J = 9.5 Hz, 1H), 7.51 (d, J = 8.8 Hz, 1H), 7.00 (dd, J = 8.8, 2.8 Hz, 1H), 6.98 (s, 1H), 3.92 - 3.81 (m, 2H), 3.46 (s, 3H), 3.36 - 3.31 (m, 6H), 3.20 (s, 3H), 3.19 - 3.09 (m, 2H), 2.97 - 2.93 (m, 1H), 2.90 (t, J = 12.2 Hz, 2H), 2.85 (s, 3H), 2.17 - 2.05 (m, 2H), 1.83 - 1.72 (m, 2H). MS (ESI) m/z: 765 [M+H]⁺. |
| I-8 | 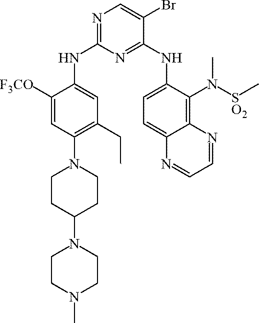 [SMILES: CCc1cc(Nc2ncc(Br)c(Nc3ccc4nccnc4c3N(C)S(C)(=O)=O)n2)c(OC(F)(F)F)cc1N1CCC(N2CCN(C)CC2)CC1] | MS (ESI) m/z: 793 [M+H]⁺. |
| I-9 | 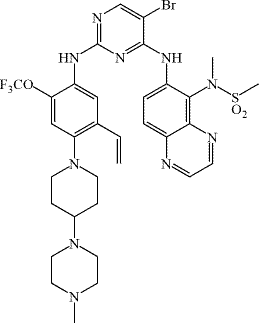 [SMILES: C=Cc1cc(Nc2ncc(Br)c(Nc3ccc4nccnc4c3N(C)S(C)(=O)=O)n2)c(OC(F)(F)F)cc1N1CCC(N2CCN(C)CC2)CC1] | 1H NMR (600 MHz, MeOD) δ 8.95 (d, J = 2.0 Hz, 1H), 8.86 (d, J = 1.8 Hz, 1H), 8.58 (s, 1H), 8.29 (s, 1H), 7.93 - 7.80 (m, 1H), 7.78 (s, 1H), 7.13 - 6.99 (m, 1H), 6.94 - 6.81 (m, 1H), 5.40 (d, J = 17.7 Hz, 1H), 5.02 (d, J = 11.3 Hz, 1H), 3.53 - 3.48 (m, 2H), 3.46 (s, 3H), 3.36 - 3.31 (m, 8H), 3.18 (s, 3H), 3.15 - 3.07 (m, 1H), 2.91 (s, 3H), 2.81 - 2.73 (m, 2H), 2.23 - 2.14 (m, 2H), 1.98 - 1.87 (m, 2H). MS (ESI) m/z: 791 [M+H]⁺. |
| I-10 | 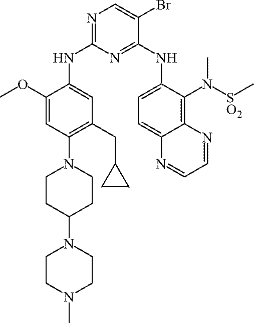 | ¹H NMR (600 MHz, MeOD) δ 8.99 (d, J = 1.9 Hz, 1H), 8.93 (d, J = 1.9 Hz, 1H), 8.54 (s, 1H), 8.35 - 8.22 (m, 1H), 8.09 - 7.93 (m, 1H), 7.59 (s, 1H), 6.86 (s, 1H), 3.85 (s, 3H), 3.72 - 3.51 (m, 8H), 3.48 (s, 3H), 3.30 - 3.26 (m, 1H), 3.25 - 3.19 (m, 2H), 3.18 (s, 3H), 2.95 (s, 3H), 2.93 - 2.76 (m, 2H), 2.44 - 2.25 (m, 2H), 2.24 - 2.17 (m, 2H), 2.02 - 1.82 (m, 2H), 0.73 - 0.51 (m, 1H), 0.34 - 0.14 (m, 2H), - 0.05 - -0.24 (m, 2H). MS (ESI) m/z: 765 [M+H]⁺. |
| I-11 | 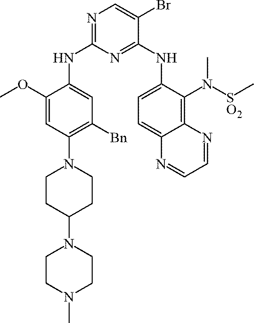 | ¹H NMR (600 MHz, MeOD) δ 9.02 - 8.90 (m, 1H), 8.86 (d, J = 6.2 Hz, 1H), 8.46 - 8.18 (m, 2H), 8.01 (s, 1H), 7.35 (s, 1H), 7.15 - 6.96 (m, 3H), 6.94 - 6.74 (m, 3H), 3.84 (s, 3H), 3.80 - 3.58 (m, 10H), 3.43 (s, 3H), 3.39 - 3.33 (m, 2H), 3.19 (s, 1H), 3.13 (s, 3H), 3.09 - 3.02 (m, 2H), 3.01 (s, 3H), 2.88 - 2.69 (m, 2H), 2.24 - 2.09 (m, 2H), 1.94 - 1.76 (m, 2H). MS (ESI) m/z: 801 [M+H]⁺. |
| I-12 | 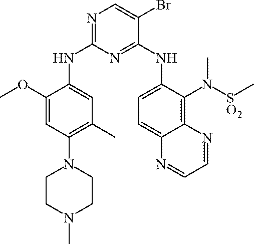 | ¹H NMR (600 MHz, MeOD) δ 8.97 (d, J = 1.8 Hz, 1H), 8.88 (d, J = 1.8 Hz, 1H), 8.68 - 8.59 (m, 1H), 8.23 (s, 1H), 8.00 (d, J = 9.3 Hz, 1H), 7.46 (s, 1H), 6.78 (s, 1H), 3.85 (s, 3H), 3.65 - 3.56 (m, 2H), 3.47 (s, 3H), 3.37 - 3.33 (m, 2H), 3.29 - 3.22 (m, 2H), 3.19 (s, 3H), 3.16 - 3.06 (m, 2H), 3.00 (s, 3H), 2.06 (s, 3H). MS (ESI) m/z: 642 [M+H]⁺. |
| I-13 | 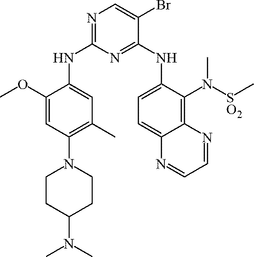 | ¹H NMR (600 MHz, MeOD) δ 8.96 (d, J = 1.9 Hz, 1H), 8.88 (d, J = 1.8 Hz, 1H), 8.73 - 8.51 (m, 1H), 8.21 (s, 1H), 7.99 (d, J = 9.1 Hz, 1H), 7.38 (s, 1H), 6.75 (s, 1H), 3.83 (s, 3H), 3.46 (s, 3H), 3.38 - 3.33 (m, 1H), 3.29 - 3.22 (m, 2H), 3.19 (s, 3H), 2.94 (s, 6H), 2.87 - 2.72 (m, 2H), 2.29 - 2.17 (m, 2H), 2.05 (s, 3H), 1.98 - 1.88 (m, 2H). MS (ESI) m/z: 670 [M+H]⁺. |
| I-14 | 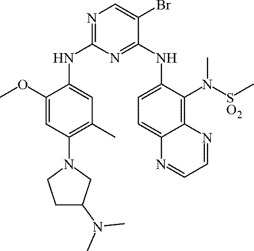 | ¹H NMR (600 MHz, MeOD) δ 8.97 (d, J = 1.7 Hz, 1H), 8.90 (d, J = 1.7 Hz, 1H), 8.72 - 8.56 (m, 1H), 8.24 (s, 1H), 8.09 - 7.94 (m, 1H), 7.31 (s, 1H), 6.73 (s, 1H), 4.11 - 3.97 (m, 1H), 3.84 (s, 3H), 3.48 (s, 3H), 3.46 - 3.38 (m, 2H), 3.19 (d, J = 3.6 Hz, 3H), 3.18 - 3.11 (m, 2H), 2.99 (s, 6H), 2.59 - 2.42 (m, 1H), 2.31 - 2.18 (m, 1H), 2.08 (s, 3H). MS (ESI) m/z: 656 [M+H]⁺. |
| I-15 | 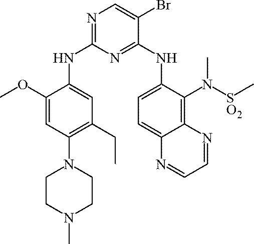 | ¹H NMR (600 MHz, MeOD) δ 8.96 (d, J = 1.9 Hz, 1H), 8.89 (d, J = 1.9 Hz, 1H), 8.66 - 8.40 (m, 1H), 8.25 (s, 1H), 7.99 (d, J = 9.4 Hz, 1H), 7.49 (s, 1H), 6.82 (s, 1H), 3.84 (s, 3H), 3.68 - 3.55 (m, 2H), 3.45 (s, 3H), 3.35 - 3.33 (m, 2H), 3.33 - 3.30 (m, 2H), 3.17 (s, 3H), 3.16 - 3.13 (m, 2H), 2.98 (s, 3H), 2.43 (d, J = 8.5 Hz, 2H), 0.97 - 0.72 (m, 3H). MS (ESI) m/z: 656 [M+H]⁺. |
| I-16 | 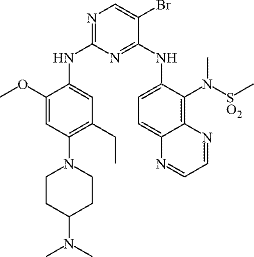 | ¹H NMR (600 MHz, MeOD) δ 8.98 (d, J = 1.8 Hz, 1H), 8.91 (d, J = 1.8 Hz, 1H), 8.67 - 8.46 (m, 1H), 8.25 (s, 1H), 8.00 (d, J = 8.7 Hz, 1H), 7.40 (s, 1H), 6.82 (s, 1H), 3.83 (s, 3H), 3.46 (s, 3H), 3.37 - 3.34 (m, 1H), 3.22 - 3.14 (m, 5H), 2.94 (s, 6H), 2.89 - 2.80 (m, 2H), 2.58 - 2.37 (m, 2H), 2.29 - 2.14 (m, 2H), 2.07 - 1.81 (m, 2H), 1.09 - 0.75 (m, 3H). MS (ESI) m/z: 684 [M+H]⁺. |
| I-17 | 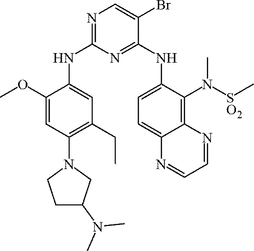 | ¹H NMR (600 MHz, MeOD) δ 9.01 - 8.96 (m, 1H), 8.94 - 8.87 (m, 1H), 8.67 - 8.43 (m, 1H), 8.26 (s, 1 H), 8.09 - 7.92 (m, 1H), 7.33 (s, 1H), 6.80 (s, 1H), 4.14 - 3.98 (m, 1H), 3.84 (s, 3H), 3.47 (s, 3H), 3.46 - 3.41 (m, 2H), 3.31 (s, 2H), 3.17 (s, 3H), 2.98 (s, 6H), 2.69 - 2.47 (m, 2H), 2.47 - 2.37 (m, 1H), 2.36 - 1.97 (m, 1H), 1.13 - 0.69 (m, 3H). MS (ESI) m/z: 670 [M+H]⁺. |
| I-18 | 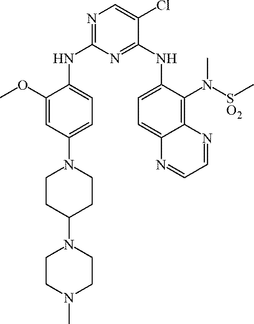 | ¹H NMR (600 MHz, MeOD) δ 9.01 - 8.95 (m, 1H), 8.93 - 8.88 (m, 1H), 8.75 - 8.65 (m, 1H), 8.20 (s, 1H), 8.11 (d, J = 9.2 Hz, 1H), 7.74 (d, J = 8.6 Hz, 1H), 7.01 (s, 1H), 6.81 (d, J = 8.9 Hz, 1H), 3.91 (s, 3H), 3.86 - 3.77 (m, 2H), 3.48 (s, 3H), 3.46 - 3.42 (m, 2H), 3.33 - 3.30 (m, 8H), 3.19 (s, 3H), 3.14 - 3.06 (m, 1H), 2.93 (s, 3H), 2.28 - 2.20 (m, 2H), 2.06 - 1.92 (m, 2H). MS (ESI) m/z: 667 [M+H]⁺. |
| I-19 | 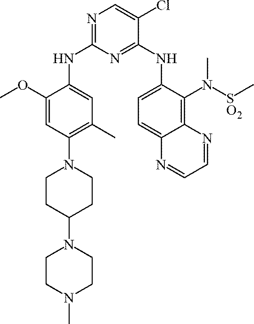 | ¹H NMR (600 MHz, MeOD) δ 9.00 (d, J = 1.8 Hz, 1H), 8.95 (d, J = 1.8 Hz, 1H), 8.56 - 8.42 (m, 1H), 8.21 (s, 1H), 8.09 (d, J = 9.3 Hz, 1H), 7.28 (s, 1H), 6.81 (s, 1H), 3.84 (s, 3H), 3.78 - 3.56 (m, 8H), 3.46 (s, 3H), 3.44 - 3.37 (m, 1H), 3.31 (s, 3H), 3.16 (s, 3H), 3.01 (s, 3H), 2.95 - 2.84 (m, 2H), 2.37 - 2.21 (m, 2H), 2.05 - 1.93 (m, 4H). MS (ESI) m/z: 681 [M+H]⁺. |
| I-20 | 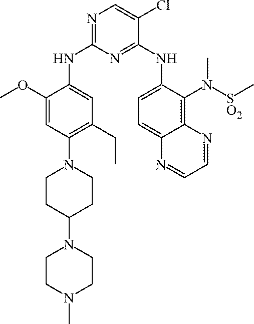 | ¹H NMR (600 MHz, MeOD) δ 8.92 (d, J = 1.8 Hz, 1H), 8.87 (d, J = 1.8 Hz, 1H), 8.46 - 8.28 (m, 1H), 8.14 (s, 1H), 8.03 - 7.93 (m, 1H), 7.26 (s, 1H), 6.76 (s, 1H), 3.76 (s, 3H), 3.69 - 3.52 (m, 8H), 3.38 (s, 3H), 3.35 - 3.29 (m, 1H), 3.16 - 3.10 (m, 2H), 3.08 (s, 3H), 2.92 (s, 3H), 2.87 - 2.70 (m, 2H), 2.48 - 2.23 (m, 2H), 2.22 - 2.13 (m, 2H), 2.01 - 1.79 (m, 2H), 0.90 - 0.65 (m, 3H). MS (ESI) m/z: 695 [M+H]⁺. |
| I-21 | 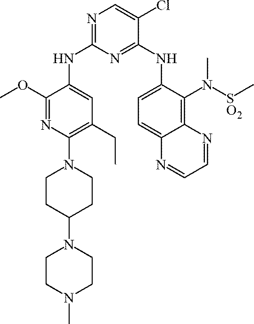 | ¹H NMR (600 MHz, MeOD) δ 9.00 (d, J = 1.8 Hz, 1H), 8.94 (d, J = 1.9 Hz, 1H), 8.54 - 8.41 (m, 1H), 8.23 (s, 1H), 8.07 (d, J = 9.3 Hz, 1H), 7.72 (s, 1H), 3.93 (s, 3H), 3.68 - 3.53 (m, 10H), 3.47 (s, 3H), 3.36 - 3.33 (m, 1H), 3.16 (s, 3H), 3.04 - 2.99 (m, 2H), 2.97 (s, 3H), 2.43 - 2.28 (m, 2H), 2.24 - 2.15 (m, 2H), 2.01 - 1.92 (m, 2H), 0.91 - 0.73 (m, 3H). MS (ESI) m/z: 696 [M+H]⁺. |
| I-22 | 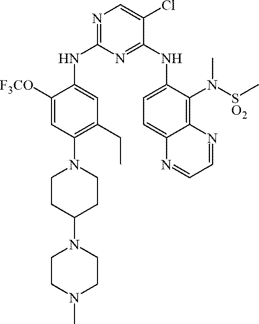 [SMILES: CCc1cc(Nc2ncc(Cl)c(Nc3ccc4nccnc4c3N(C)S(C)(=O)=O)n2)c(OC(F)(F)F)cc1N1CCC(N2CCN(C)CC2)CC1] | ¹H NMR (600 MHz, MeOD) δ 8.96 (d, J = 1.8 Hz, 1H), 8.88 (d, J = 1.9 Hz, 1H), 8.54 (d, J = 9.3 Hz, 1H), 8.21 (s, 1H), 7.92 (d, J = 9.3 Hz, 1H), 7.52 (s, 1H), 7.07 (s, 1H), 3.63 - 3.50 (m, 8H), 3.44 (s, 3H), 3.28 - 3.23 (m, 1H), 3.21 - 3.18 (m, 2H), 3.17 (s, 3H), 2.95 (s, 3H), 2.83 - 2.74 (m, 2H), 2.59 - 2.46 (m, 2H), 2.27 - 2.18 (m, 2H), 1.99 - 1.89 (m, 2H), 0.93 (t, J = 7.4 Hz, 3H). MS (ESI) m/z: 749 [M+H]⁺. |
| I-23 | 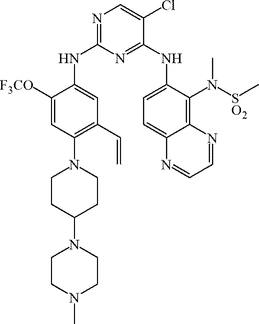 [SMILES: C=Cc1cc(Nc2ncc(Cl)c(Nc3ccc4nccnc4c3N(C)S(C)(=O)=O)n2)c(OC(F)(F)F)cc1N1CCC(N2CCN(C)CC2)CC1] | ¹H NMR (600 MHz, Methanol-d₄) δ 8.95 (d, J = 1.7 Hz, 1H), 8.87 (d, J = 1.8 Hz, 1H), 8.63 - 8.43 (m, 1H), 8.22 (s, 1H), 7.86 (d, J = 9.4 Hz, 1H), 7.77 (s, 1H), 7.02 (s, 1H), 6.85 (dd, J = 17.7, 11.0 Hz, 1H), 5.37 (d, J = 17.7 Hz, 1H), 4.99 (d, J = 11.0 Hz, 2H), 3.73 - 3.48 (m, 10H), 3.45 (s, 3H), 3.25 - 3.21 (m, 1H), 3.17 (s, 3H), 2.95 (s, 3H), 2.81 - 2.71 (m, 2H), 2.25 - 2.16 (m, 2H), 2.00 - 1.89 (m, 2H). MS (ESI) m/z: 747 [M+H]⁺. |
| I-24 | 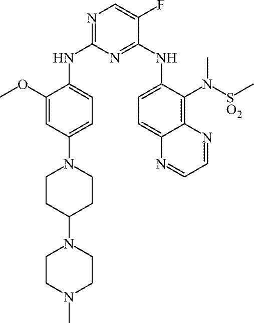 | ¹H NMR (600 MHz, MeOD) δ 8.98 (d, J = 1.9 Hz, 1H), 8.92 (d, J = 1.9 Hz, 1H), 8.58 (d, J = 9.3 Hz, 1H), 8.14 (d, J = 9.3 Hz, 1H), 8.10 (d, J = 4.2 Hz, 1H), 7.72 - 7.64 (m, 1H), 6.99 - 6.91 (m, 1H), 6.75 (d, J = 8.4 Hz, 1H), 3.89 (s, 3H), 3.84 - 3.78 (m, 2H), 3.49 (s, 3H), 3.43 - 3.31 (m, 8H), 3.23 - 3.19 (m, 2H), 3.15 (s, 3H), 3.01 - 2.96 (m, 1H), 2.89 (s, 3H), 2.22 - 2.14 (m, 2H), 1.97 - 1.88 (m, 2H). MS (ESI) m/z: 651 [M+H]⁺. |
| I-25 | 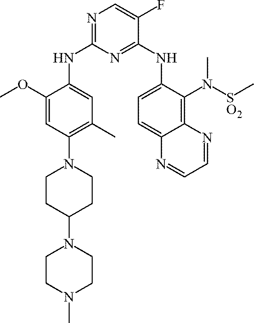 | ¹H NMR (600 MHz, MeOD) δ 8.94 (d, J = 1.8 Hz, 1H), 8.90 (d, J = 1.7 Hz, 1H), 8.28 (d, J = 9.2 Hz, 1H), 8.07 (s, 1H), 8.06 - 8.04 (m, 1H), 7.21 (s, 1H), 6.72 (s, 1H), 3.78 (s, 3H), 3.69 - 3.50 (m, 8H), 3.39 (s, 3H), 3.30 - 3.27 (m, 1H), 3.24 - 3.16 (m, 2H), 3.08 (s, 3H), 2.92 (s, 3H), 2.86 - 2.76 (m, 2H), 2.24 - 2.11 (m, 2H), 1.95 - 1.88 (m, 2H), 1.84 (s, 3H). MS (ESI) m/z: 665 [M+Hr]⁺. |
| I-26 | 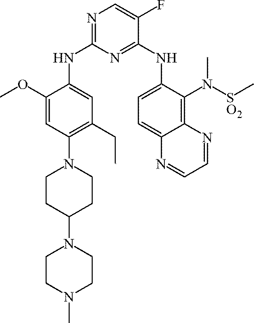 | ¹H NMR (600 MHz, MeOD) δ 8.99 (d, J = 1.9 Hz, 1H), 8.94 (d, J = 1.9 Hz, 1H), 8.41 (d, J = 9.3 Hz, 1H), 8.11 (s, 1H), 8.10 - 8.09 (m, 1H), 7.43 (s, 1H), 6.81 (s, 1H), 3.84 (s, 3H), 3.46 (s, 3H), 3.39 - 3.33 (m, 8H), 3.22 - 3.15 (m, 4H), 3.13 (s, 3H), 2.95 - 2.91 (m, 1H), 2.90 - 2.84 (m, 2H), 2.82 (s, 3H), 2.14 - 2.04 (m, 2H), 1.94 - 1.74 (m, 2H), 0.87 - 0.73 (m, 3H). MS (ESI) m/z: 679 [M+H]⁺. |
| I-27 | 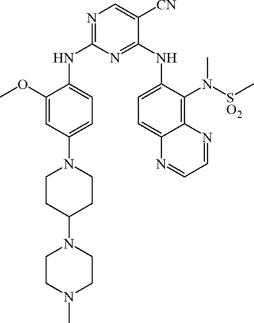 | ¹H NMR (600 MHz, DMSO-d₆) δ 9.25 (s, 1H), 9.02 - 8.96 (m, 1H), 8.95 - 8.86 (m, 1H), 8.81 - 8.64 (m, 1H), 8.63 - 8.34 (m, 2H), 8.03 - 7.74 (m, 1H), 7.29 - 7.06 (m, 1H), 6.71 (s, 1H), 6.47 (s, 1H), 3.91 - 3.87 (m, 2H), 3.76 (s, 3H), 3.60 - 3.44 (m, 5H), 3.33 (s, 3H), 3.18 (s, 3H), 3.14 - 2.88 (m, 4H), 2.80 (s, 3H), 2.75 (t, J = 12.6 Hz, 2H), 2.11 - 1.98 (m, 2H), 1.71 - 1.59 (m, 2H). MS (ESI) m/z: 658 [M+H]⁺. |
| I-28 | 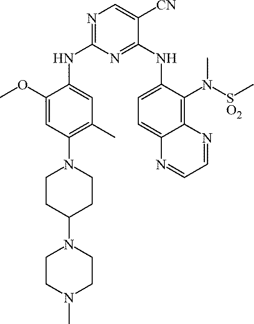 | ¹H NMR (600 MHz, Methanol-d₄) δ 8.93 (d, J = 1.8 Hz, 1H), 8.83 (d, J = 1.9 Hz, 1H), 8.63 - 8.46 (m, 1H), 8.38 (s, 1H), 8.09 - 7.80 (m, 1H), 7.61 - 7.27 (m, 1H), 6.73 (s, 1H), 3.81 (s, 3H), 3.72 (p, J = 6.6 Hz, 1H), 3.44 (s, 3H), 3.28 - 3.18 (m, 9H), 3.16 (s, 3H), 2.97 (s, 1H), 2.86 - 2.75 (m, 5H), 2.13 - 2.06 (m, 2H), 2.03 - 1.75 (m, 5H). MS (ESI) m/z: 672 [M+H]⁺. |
| I-29 | 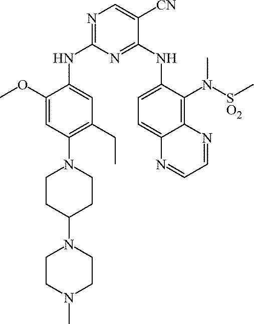 | ¹H NMR (600 MHz, Methanol-d₄) δ 9.01 - 8.89 (m, 1H), 8.90 - 8.74 (m, 1H), 8.53 - 8.31 (m, 2H), 8.09 - 7.80 (m, 1H), 7.68 - 7.34 (m, 1H), 6.80 (s, 1H), 3.82 (s, 3H), 3.72 (p, J = 6.6 Hz, 1H), 3.44 (s, 3H), 3.34 - 3.20 (m, 7H), 3.16 - 3.10 (m, 5H), 3.03 - 2.94 (m, 1H), 2.87 - 2.78 (m, 5H), 2.42 - 2.13 (m, 2H), 2.15 - 2.05 (m, 2H), 1.93 - 1.76 (m, 2H), 0.65 (s, 3H). MS (ESI) m/z: 686 [M+H]⁺. |
| I-30 | 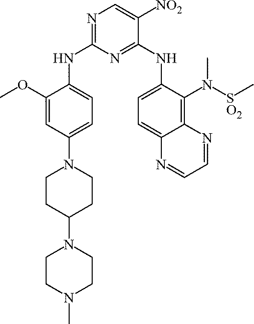 | ¹H NMR (600 MHz, DMSO-d₆) δ 11.31 (s, 1H), 9.89 (s, 1H), 9.14 (s, 1H), 9.01 - 8.94 (m, 1H), 8.95 - 8.86 (m, 1H), 8.73 (d, J = 9.4 Hz, 1H), 7.81 (d, J = 9.4 Hz, 1H), 7.23 (d, J = 8.5 Hz, 1H), 6.81 (s, 1H), 6.62 (d, J = 8.8 Hz, 1H), 4.16 - 3.89 (m, 10H), 3.78 (s, 3H), 3.45 - 3.42 (m, 1H), 3.36 (s, 3H), 3.21 (s, 3H), 2.86 (s, 3H), 2.84 - 2.78 (m, 2H), 2.22 - 2.04 (m, 2H), 1.80 - 1.65 (m, 2H). MS (ESI) m/z: 678 [M+H]⁺. |
| I-31 | 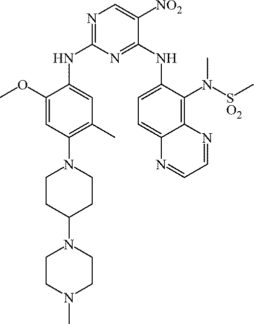 | ¹H NMR (600 MHz, Chloroform-d) δ 11.51 (s, 1H), 9.18 (s, 1H), 8.95 - 8.87 (m, 2H), 8.82 (d, J = 9.3 Hz, 1H), 8.13 (s, 1H), 8.03 (d, J = 9.2 Hz, 1H), 7.79 (s, 1H), 6.58 (s, 1H), 3.86 (s, 3H), 3.80 - 3.63 (m, 8H), 3.52 (s, 3H), 3.33 - 3.26 (m, 2H), 3.27 - 3.22 (m, 1H), 3.19 (s, 3H), 2.91 (s, 3H), 2.79 - 2.72 (m, 2H), 2.22 - 2.15 (m, 2H), 2.02 - 1.95 (m, 2H), 1.91 (s, 3H). MS (ESI) m/z: 692 [M+H]⁺. |
| I-32 | 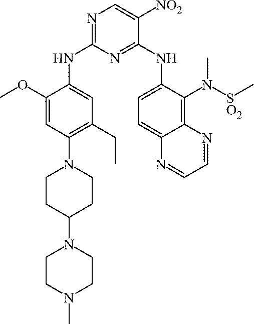 | ¹H NMR (600 MHz, DMSO-d₆) δ 11.23 (s, 1H), 9.84 (s, 1H), 9.16 (s, 1H), 9.03 - 8.97 (m, 1H), 8.96 - 8.87 (m, 1H), 8.59 (d, J = 9.4 Hz, 1H), 7.71 (d, J = 9.4 Hz, 1H), 7.21 (s, 1H), 6.88 (s, 1H), 3.76 (s, 3H), 3.70 - 3.59 (m, 8H), 3.36 (s, 3H), 3.25 (s, 1H), 3.21 (s, 3H), 3.17 - 3.12 (m, 4H), 2.84 (s, 3H), 2.49 - 2.40 (m, 2H), 2.15 - 2.05 (m, 2H), 1.81 - 1.72 (m, 2H), 0.92 (t, J = 7.4 Hz, 3H). MS (ESI) m/z: 706 [M+H]⁺. |
| I-33 | 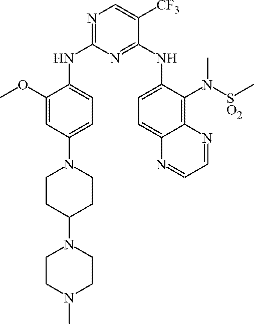 | ¹H NMR (600 MHz, MeOD) δ 8.97 (d, J = 1.9 Hz, 1H), 8.91 (d, J = 1.9 Hz, 1H), 8.63 - 8.55 (m, 1H), 8.42 (s, 1H), 8.08 (d, J = 9.2 Hz, 1H), 7.84 - 7.77 (m, 1H), 7.06 - 7.01 (m, 1H), 6.76 (s, 1H), 3.90 (s, 3H), 3.82 - 3.76 (m, 2H), 3.43 (s, 3H), 3.43 - 3.38 (m, 2H), 3.35 - 3.28 (m, 8H), 3.15 (s, 3H), 3.06 - 3.01 (m, 1H), 2.92 (s, 3H), 2.21 (d, J = 12.6 Hz, 2H), 1.99 (tt, J = 12.1, 6.2 Hz, 2H). MS (ESI) m/z: 701 [M+H]⁺. |
| I-34 | 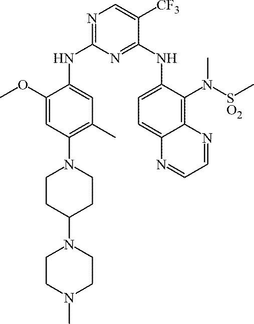 | ¹H NMR (600 MHz, MeOD) δ 8.98 (d, J = 1.7 Hz, 1H), 8.92 (d, J = 1.7 Hz, 1H), 8.54 - 8.32 (m, 2H), 8.13 - 7.96 (m, 1H), 7.38 (s, 1H), 6.80 (s, 1H), 3.84 (s, 3H), 3.73 - 3.52 (m, 8H), 3.42 (s, 3H), 3.35 - 3.33 (m, 1H), 3.31 - 3.24 (m, 2H), 3.14 (s, 3H), 2.98 (s, 3H), 2.95 - 2.88 (m, 2H), 2.32 - 2.18 (m, 2H), 2.08 - 1.97 (m, 2H), 1.93 (s, 3H). MS (ESI) m/z: 715 [M+H]⁺. |
| I-35 | 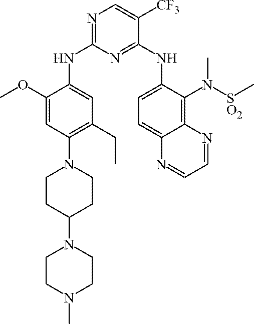 | ¹H NMR (600 MHz, MeOD) δ 8.95 (d, J = 2.0 Hz, 1H), 8.88 (d, J = 2.0 Hz, 1H), 8.50 - 8.27 (m, 2H), 7.97 (s, 1H), 7.46 (s, 1H), 6.80 (s, 1H), 3.81 (s, 3H), 3.60 - 3.42 (m, 8H), 3.40 (s, 3H), 3.22 - 3.17 (m, 2H), 3.17 - 3.13 (m, 1H), 3.11 (s, 3H), 2.93 - 2.91 (m, 3H), 2.91 - 2.85 (m, 2H), 2.41 - 2.18 (m, 2H), 2.19 - 2.12 (m, 2H), 1.97 - 1.86 (m, 2H), 0.97 - 0.30 (m, 3H). MS (ESI) m/z: 729 [M+H]⁺. |
| I-36 | 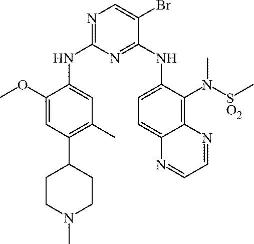 | MS (ESI) m/z: 641 [M+H]⁺. |
| I-37 | 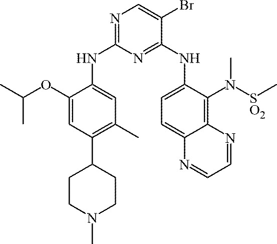 [SMILES: Cc1cc(Nc2ncc(Br)c(Nc3ccc4nccnc4c3N(C)S(C)(=O)=O)n2)c(OC(C)C)cc1C1CCN(C)CC1] | ¹H NMR (600 MHz, DMSO-d₆) δ 9.01 - 8.98 (m, 1H), 8.97 - 8.92 (m, 1H), 8.81 (d,J = 9.1 Hz, 1H), 8.70 (s, 1H), 8.36 (s, 1H), 8.24 (s, 1H), 7.99 (d, J = 9.3 Hz, 1H), 7.50 (s, 1H), 6.79 (s, 1H), 4.55 - 4.49 (m, 1H), 3.37 (s, 3H), 3.23 (s, 3H), 3.19 - 3.08 (m, 4H), 3.02 - 2.90 (m, 1H), 2.83 (s, 3H), 2.05 (s, 3H), 1.94 - 1.87 (m, 4H), 1.26 - 1.22 (m, 6H). MS (ESI) m/z: 669 [M+H]⁺. |
| I-38 | 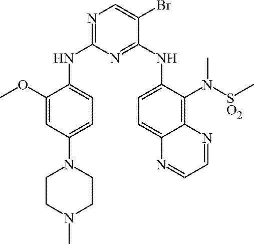 | ¹H NMR (600 MHz, DMSO-d₆) δ 9.88 - 9.70 (m, 1H), 8.97 (d, J = 1.9 Hz, 1H), 8.91 (d, J = 1.9 Hz, 1H), 8.62 (s, 1H), 8.58 (s, 1H), 8.30 (s, 1H), 8.04 - 7.93 (m, 1H), 7.39 (d, J = 8.8 Hz, 1H), 6.77 (d, J = 2.6 Hz, 1H), 6.57 (d, J = 8.8, 2.7 Hz, 1H), 3.95 - 3.90 (m, 2H), 3.79 (s, 3H), 3.61 - 3.54 (m, 2H), 3.38 (s, 3H), 3.25 (s, 3H), 3.23 - 3.17 (m, 2H), 3.05 - 2.94 (m, 2H), 2.90 (s, 3H). MS (ESI) m/z: 628 [M+H]⁺. |
| I-39 | 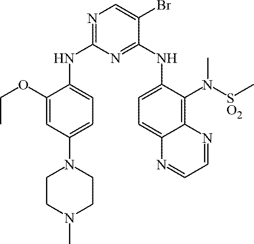 [SMILES: CCOc1cc(N2CCN(C)CC2)ccc1Nc1ncc(Br)c(Nc2ccc3nccnc3c2N(C)S(C)(=O)=O)n1] | ¹H NMR (600 MHz, DMSO-d₆) δ 9.78 - 9.68 (m, 1H), 8.98 (d, J = 1.8 Hz, 1H), 8.92 (d, J = 1.8 Hz, 1H), 8.64 (s, 1H), 8.49 (s, 1H), 8.31 (s, 1H), 8.00 - 7.93 (m, 1H), 7.41 (d, J = 8.7 Hz, 1H), 6.75 (d, J = 2.5 Hz, 1H), 6.55 (d, J = 8.7, 2.5 Hz, 1H), 4.09 - 4.02 (m, 2H), 3.95 - 3.85 (m, 2H), 3.59 - 3.52 (m, 2H), 3.38 (s, 3H), 3.24 (s, 3H), 3.22 - 3.15 (m, 2H), 3.00 - 2.93 (m, 2H), 2.89 (s, 3H), 1.25 (t, J = 6.9 Hz, 3H). MS (ESI) m/z: 642 [M+H]⁺. |
| I-40 | 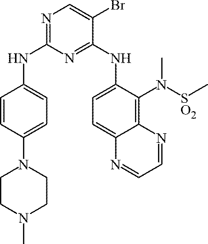 [SMILES: CN1CCN(c2ccc(Nc3ncc(Br)c(Nc4ccc5nccnc5c4N(C)S(C)(=O)=O)n3)cc2)CC1] | ¹H NMR (600 MHz, DMSO-d₆) δ 9.35 (s, 1H), 8.98 (d, J = 1.8 Hz, 1H), 8.94 (d, J = 1.8 Hz, 1H), 8.61 (s, 1H), 8.34 (s, 1H), 8.10 (d, J = 9.5 Hz, 1H), 7.45 - 7.41 (m, 3H), 6.90 - 6.87 (m, 2H), 3.39 (s, 3H),3.25 (s, 3H), 3.11 - 3.06 (m, 4H), 2.49 - 2.46 (m, 4H), 2.24 (s, 3H). MS (ESI) m/z: 598 [M+H]⁺. |
| 1-41 | 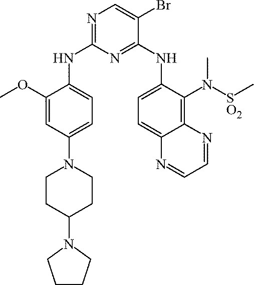 | ¹H NMR (600 MHz, DMSO-d₆) δ 8.95 (d, J = 1.9 Hz, 1H), 8.89 (d, J = 1.8 Hz, 1H), 8.55 (s, 1H), 8.43 (s, 1H), 8.27 (s, 1H), 7.98 - 7.88 (m, 1H), 7.36 - 7.26 (m, 1H), 6.70 - 6.66 (m, 1H), 6.51 - 6.46 (m, 1H), 3.77 (s, 3H), 3.66 (d, J = 12.2 Hz, 2H), 3.37 (s, 3H), 3.24 (s, 3H), 2.82 - 2.68 (m, 2H), 2.58 - 2.52 (m, 4H), 2.17 - 2.09 (m, 1H), 1.98 - 1.90 (m, 2H), 1.72 - 1.65 (m, 4H), 1.58 - 1.50 (m, 2H). MS (ESI) m/z: 682 [M+H]⁺. |
| I-42 | 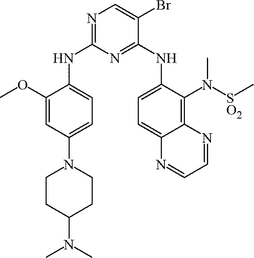 | MS (ESI) m/z: 656 [M+H]⁺. |
| I-43 | 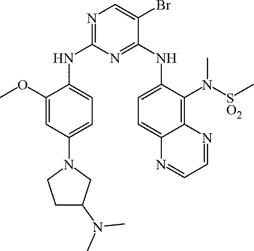 | ¹H NMR (600 MHz, DMSO-d₆) δ 10.45 (s, 1H), 9.01 - 8.95 (m, 1H), 8.95 - 8.89 (m, 2H), 8.87 - 8.80 (m, 1H), 8.31 (s, 1H), 8.03 - 7.88 (m, 1H), 7.23 (d, J = 8.5 Hz, 1H), 6.38 - 6.31 (m, 1H), 6.19 (dd, J = 8.5, 2.3 Hz, 1H), 4.10 - 4.00 (m, 1H), 3.78 (s, 3H), 3.69 - 3.62 (m, 1H), 3.57 - 3.48 (m, 2H), 3.37 (s, 3H), 3.32 - 3.26 (m, 1H), 3.23 (s, 3H), 2.90 (s, 6H), 2.49 - 2.44 (m, 1H), 2.28 - 2.19 (m, 1H). MS (ESI) m/z: 642 [M+H]⁺. |
| I-44 | 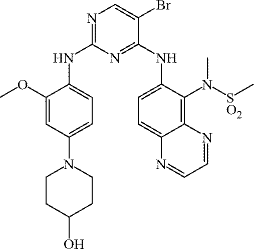 | ¹H NMR (600 MHz, DMSO-d₆) δ 8.98 (d, J = 1.9 Hz, 1H), 8.94 (d, J = 1.8 Hz, 1H), 8.90 - 8.84 (m, 1H), 8.73 (s, 1H), 8.70 (s, 1H), 8.36 (s, 1H), 8.06 (d, J = 9.4 Hz, 1H), 7.72 - 7.62 (m, 1H), 7.15 - 7.10 (m, 1H), 6.96 - 6.90 (m, 1H), 3.87 - 3.79 (m, 4H), 3.68 - 3.56 (m, 2H), 3.38 (s, 3H), 3.36 - 3.27 (m, 2H), 3.23 (s, 3H), 2.05 - 1.95 (m, 2H), 1.78 - 1.65 (m, 2H). MS (ESI) m/z: 629 [M+H]⁺. |
| I-45 | 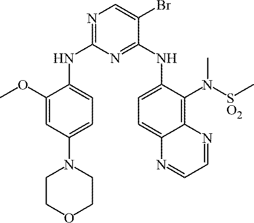 | ¹H NMR (600 MHz, DMSO-d₆) δ 9.06 (s, 1H), 9.01 - 8.94 (m, 3H), 8.76 (s, 1H), 8.35 (s, 1H), 8.07 - 7.97 (m, 1H), 7.32 (d, J = 8.7 Hz, 1H), 6.74 (d, J = 2.5 Hz, 1H), 6.51 (dd, J = 8.7, 2.5 Hz, 1H), 3.82 - 3.79 (m, 4H), 3.79 (s, 3H), 3.38 (s, 3H), 3.23 (s, 3H), 3.19 (t, J = 4.9 Hz, 4H). MS (ESI) m/z: 615 [M+H]⁺. |
| I-46 | 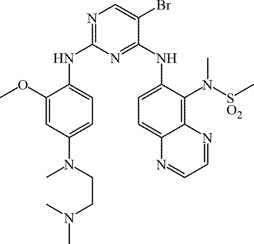 | MS (ESI) m/z: 630 [M+H]⁺. |
| I-47 | 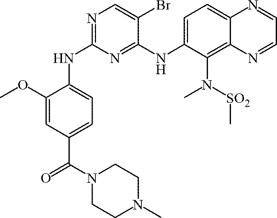 [SMILES: COc1cc(C(=O)N2CCN(C)CC2)ccc1Nc1ncc(Br)c(Nc2ccc3nccnc3c2N(C)S(C)(=O)=O)n1] | MS (ESI) m/z: 656 [M+H]⁺. |
| I-48 | 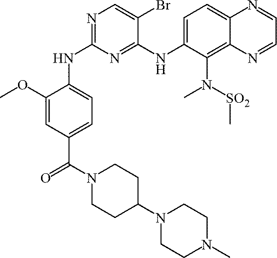 [SMILES: COc1cc(C(=O)N2CCC(N3CCN(C)CC3)CC2)ccc1Nc1ncc(Br)c(Nc2ccc3nccnc3c2N(C)S(C)(=O)=O)n1] | ¹H NMR (600 MHz, DMSO-d₆) δ 9.03 - 8.98 (m, 1H), 8.96 - 8.93 (m, 1H), 8.88 (d, J = 9.4 Hz, 1H), 8.76 (s, 1H), 8.73 (s, 1H), 8.43 (s, 1H), 8.08 (d, J = 9.3 Hz, 1H), 7.85 (d, J = 8.1 Hz, 1H), 7.12 (s, 1H), 6.96 (d, J = 8.2 Hz, 1H), 3.87 (s, 3H), 3.52 (s, 9H), 3.40 (s, 3H), 3.25 (s, 3H), 3.20 - 2.98 (m, 4H), 2.90 (s, 3H), 2.20 - 1.97 (m, 2H), 1.72 - 1.57 (m, 2H). MS (ESI) m/z: 739 [M+H]⁺. |
| I-49 | 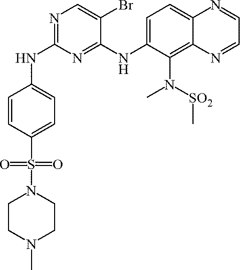 [SMILES: CN1CCN(S(=O)(=O)c2ccc(Nc3ncc(Br)c(Nc4ccc5nccnc5c4N(C)S(C)(=O)=O)n3)cc2)CC1] | ¹H NMR (600 MHz, DMSO-d₆) δ 10.20 (s, 1H), 9.02 (d, J = 1.9 Hz, 1H), 8.99 (d, J = 1.9 Hz, 1H), 8.96 (d, J = 9.5 Hz, 1H), 8.79 (s, 1H), 8.51 (s, 1H), 8.22 (d, J = 9.3 Hz, 1H), 7.95 (d, J = 8.5 Hz, 2H), 7.62 (d, J = 8.5 Hz, 2H), 3.84 - 3.65 (m, 2H), 3.59 - 3.46 (m, 2H), 3.41 (s, 3H), 3.25 (s, 3H), 3.20 - 3.11 (m, 2H), 2.80 (s, 3H), 2.58 - 2.45 (m, 2H). MS (ESI) m/z: 662 [M+H]⁺. |
| I-50 | 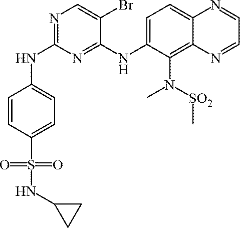 [SMILES: CN(c1c(Nc2nc(Nc3ccc(S(=O)(=O)NC4CC4)cc3)ncc2Br)ccc2nccnc12)S(C)(=O)=O] | ¹H NMR (600 MHz, DMSO-d₆) δ 10.04 (s, 1H), 9.00 (d, 1H), 8.97 (d, J = 1.8 Hz, 1H), 8.94 (d, J = 9.3 Hz, 1H), 8.73 (s, 1H), 8.48 (s, 1H), 8.17 (d, J = 9.3 Hz, 1H), 7.84 (d, J = 8.4 Hz, 2H), 7.73 (d, J = 2.7 Hz, 1H), 7.64 (d, J = 8.4 Hz, 2H), 3.39 (s, 3H), 3.23 (s, 3H), 2.09 - 2.03 (m, 1H), 0.51 - 0.42 (m, 2H), 0.40 - 0.32 (m, 2H). MS (ESI) m/z: 619 [M+H]⁺. |
| I-51 | 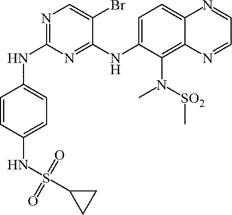 [SMILES: CN(c1c(Nc2nc(Nc3ccc(NS(=O)(=O)C4CC4)cc3)ncc2Br)ccc2nccnc12)S(C)(=O)=O] | MS (ESI) m/z: 619 [M+H]⁺. |
| I-52 | 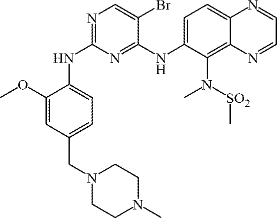 [SMILES: COc1cc(CN2CCN(C)CC2)ccc1Nc1ncc(Br)c(Nc2ccc3nccnc3c2N(C)S(C)(=O)=O)n1] | MS (ESI) m/z: 642 [M+H]⁺. |
| I-53 | 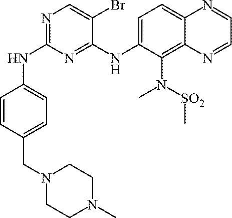 [SMILES: CN1CCN(Cc2ccc(Nc3ncc(Br)c(Nc4ccc5nccnc5c4N(C)S(C)(=O)=O)n3)cc2)CC1] | MS (ESI) m/z: 612 [M+H]⁺. |
| I-54 | 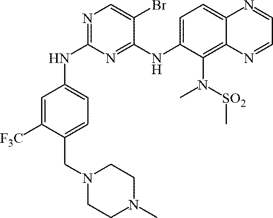 [SMILES: CN1CCN(Cc2ccc(Nc3ncc(Br)c(Nc4ccc5nccnc5c4N(C)S(C)(=O)=O)n3)cc2C(F)(F)F)CC1] | MS (ESI) m/z: 680 [M+H]⁺. |
| I-55 | 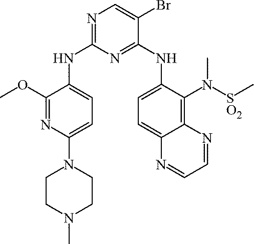 | ¹H NMR (600 MHz, DMSO-d₆) δ 10.36 (s, 1H), 8.99 (d, J = 1.9 Hz, 1H), 8.94 (d, J = 1.8 Hz, 1H), 8.93 - 8.90 (m, 1H), 8.77 (s, 1H), 8.34 (s, 1H), 8.05 - 7.92 (m, 1H), 7.69 (d, J = 8.3 Hz, 1H), 6.51 (d, J = 8.4 Hz, 1H), 4.45 - 4.36 (m, 2H), 3.84 (s, 3H), 3.62 - 3.53 (m, 2H), 3.40 (s, 3H), 3.25 (s, 3H), 3.21 - 3.09 (m, 4H), 2.90 (s, 3H). MS (ESI) m/z: 629 [M+H]⁺. |
| I-56 | 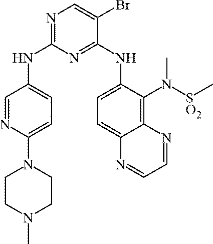 [SMILES: CN1CCN(c2ccc(Nc3ncc(Br)c(Nc4ccc5nccnc5c4N(C)S(C)(=O)=O)n3)cn2)CC1] | ¹H NMR (600 MHz, DMSO-d₆) δ 9.34 (s, 1H), 9.01 - 8.96 (m, 1H), 8.94 - 8.92 (m, 1H), 8.61 (s, 1H), 8.33 (s, 1H), 8.30 - 8.24 (m, 1H), 8.14 - 7.96 (m, 1H), 7.84 - 7.69 (m, 1H), 6.82 (d, J = 8.8 Hz, 1H), 3.49 - 3.42 (m, 4H), 3.25 (s, 3H), 3.18 (s, 3H), 2.45 - 2.37 (m, 4H), 2.23 (s, 3H). MS (ESI) m/z: 599 [M+H]⁺. |
| I-57 | 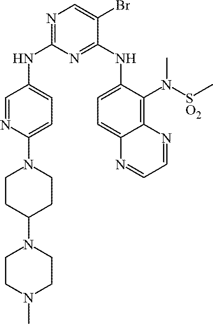 [SMILES: CN1CCN(C2CCN(c3ccc(Nc4ncc(Br)c(Nc5ccc6nccnc6c5N(C)S(C)(=O)=O)n4)cn3)CC2)CC1] | MS (ESI) m/z: 682 [M+H]⁺. |
| I-58 | 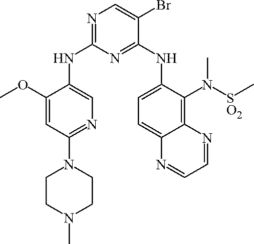 | MS (ESI) m/z: 629 [M+H]⁺. |
| I-59 | 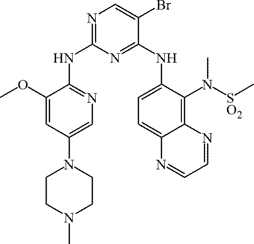 | MS (ESI) m/z: 629 [M+H]⁺. |
| I-60 | 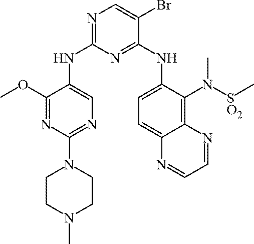 | MS (ESI) m/z: 630 [M+H]⁺. |
| I-61 | 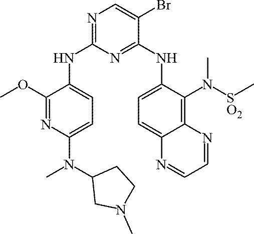 | MS (ESI) m/z: 643 [M+H]⁺. |
| I-62 | 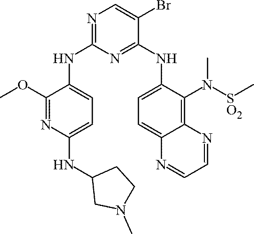 | MS (ESI) m/z: 629 [M+H]⁺. |
| I-63 | 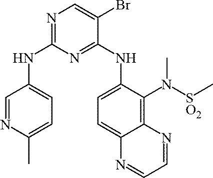 [SMILES: Cc1ccc(Nc2ncc(Br)c(Nc3ccc4nccnc4c3N(C)S(C)(=O)=O)n2)cn1] | ¹H NMR (600 MHz, DMSO-d₆) δ 10.40 (s, 1H), 9.10 (s, 1H), 9.02 (d, J = 1.9 Hz, 1H), 8.98 (d, J = 1.9 Hz, 1H), 8.89 (d, J = 9.3 Hz, 1H), 8.82 (s, 1H), 8.55 - 8.51 (m, 2H), 8.22 (d, J = 9.3 Hz, 1H), 7.81 (d, J = 8.8 Hz, 1H), 3.43 (s, 3H), 3.25 (s, 3H), 2.65 (s, 3H). MS (ESI) m/z: 515 [M+H]⁺. |
| I-64 | 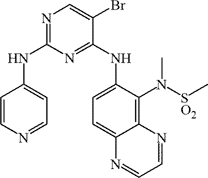 [SMILES: CN(c1c(Nc2nc(Nc3ccncc3)ncc2Br)ccc2nccnc12)S(C)(=O)=O] | MS (ESI) m/z: 501 [M+H]⁺. |
| I-65 | 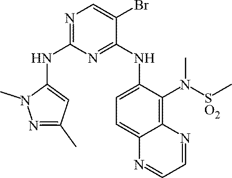 | MS (ESI) m/z: 518 [M+H]⁺. |
| I-66 | 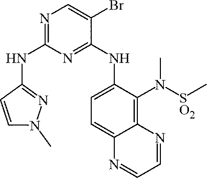 [SMILES: CN(c1c(Nc2nc(Nc3ccn(C)n3)ncc2Br)ccc2nccnc12)S(C)(=O)=O] | ¹H NMR (600 MHz, DMSO-d₆) δ 10.15 - 10.02 (m, 1H), 9.20 (d, J = 9.3 Hz, 1H), 8.99 (d, J = 1.9 Hz, 1H), 8.95 (d, J = 1.9 Hz, 1H), 8.84 (s, 1H), 8.39 (s, 1H), 8.11 (d, J = 9.3 Hz, 1H), 7.58 (d, J = 2.2 Hz, 1H), 6.30 (s, 1H), 3.79 (s, 3H), 3.41 (s, 3H), 3.25 (s, 3H). MS (ESI) m/z: 504 [M+H]⁺. |
| I-67 | 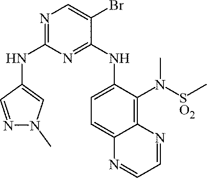 [SMILES: CN(c1c(Nc2nc(Nc3cnn(C)c3)ncc2Br)ccc2nccnc12)S(C)(=O)=O] | ¹H NMR (600 MHz, DMSO-d₆) δ 9.73 - 9.51 (m, 1H), 9.19 (s, 1H), 9.06 - 8.99 (m, 1H), 8.98 - 8.94 (m, 1H), 8.88 - 8.71 (m, 1H), 8.40 - 8.34 (m, 1H), 8.19 (d, J = 9.3 Hz, 1H), 7.95 - 7.81 (m, 1H), 7.52 - 7.30 (m, 1H), 3.85 - 3.55 (m, 3H), 3.39 (s, 3H), 3.24 (s, 3H). MS (ESI) m/z: 504 [M+H]⁺. |
| I-68 | 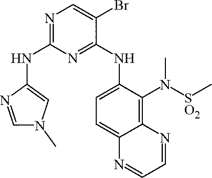 [SMILES: CN(c1c(Nc2nc(Nc3cn(C)cn3)ncc2Br)ccc2nccnc12)S(C)(=O)=O] | MS (ESI) m/z: 504 [M+H]⁺. |
| I-69 | 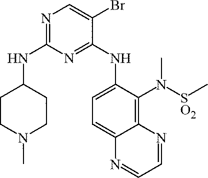 [SMILES: CN1CCC(Nc2ncc(Br)c(Nc3ccc4nccnc4c3N(C)S(C)(=O)=O)n2)CC1] | MS (ESI) m/z: 521 [M+H]⁺. |
| I-70 | 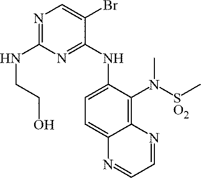 [SMILES: CN(c1c(Nc2nc(NCCO)ncc2Br)ccc2nccnc12)S(C)(=O)=O] | ¹H NMR (600 MHz, DMSO-d₆) δ 9.29 - 9.11 (m, 1H), 8.97 (d, J = 1.9 Hz, 1H), 8.92 (d, J = 1.8 Hz, 1H), 8.53 (s, 1H), 8.22 (s, 1H), 8.14 (s, 1H), 7.24 (t, J = 5.8 Hz, 1H), 4.73 (t, J = 5.4 Hz, 1H), 3.59 - 3.51 (m, 4H), 3.40 (s, 3H), 3.26 (s, 3H). MS (ESI) m/z: 468 [M+H]⁺. |
| I-71 | 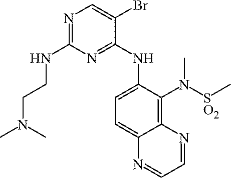 | ¹H NMR (600 MHz, DMSO-d₆) δ 9.53 (s, 1H), 9.00 (d, J = 1.9 Hz, 1H), 8.95 (s, 1H), 8.88 - 8.76 (m, 1H), 8.32 (s, 1H), 8.22 - 8.11 (m, 1H), 7.76 - 7.48 (m, 1H), 3.65 - 3.55 (m, 2H), 3.41 (s, 3H), 3.29 - 3.21 (m, 5H), 2.96 - 2.64 (m, 6H). MS (ESI) m/z: 495 [M+H]⁺. |
| I-72 | 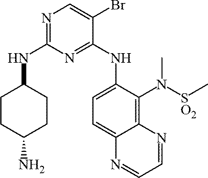 [SMILES: CN(c1c(Nc2nc(N[C@H]3CC[C@H](N)CC3)ncc2Br)ccc2nccnc12)S(C)(=O)=O] | MS (ESI) m/z: 521 [M+H]⁺. |
| I-73 | 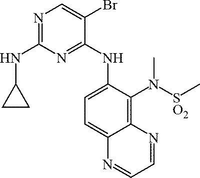 [SMILES: CN(c1c(Nc2nc(NC3CC3)ncc2Br)ccc2nccnc12)S(C)(=O)=O] | ¹H NMR (600 MHz, DMSO-d₆) δ 9.50 - 9.36 (m, 1H), 9.04 - 8.98 (m, 1H), 8.99 - 8.93 (m, 1H), 8.89 - 8.79 (m, 1H), 8.43 - 8.28 (m, 2H), 8.25 - 8.15 (m, 1H), 3.42 (s, 3H), 3.26 (s, 3H), 2.76 - 2.62 (m, 1H), 0.89 - 0.73 (m, 2H), 0.62 - 0.53 (m, 2H). MS (ESI) m/z: 464 [M+H]⁺. |
| I-74 | 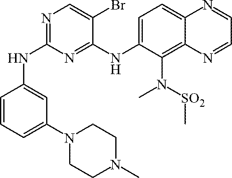 [SMILES: CN1CCN(c2cccc(Nc3ncc(Br)c(Nc4ccc5nccnc5c4N(C)S(C)(=O)=O)n3)c2)CC1] | MS (ESI) m/z: 598 [M+H]⁺. |
| I-75 | 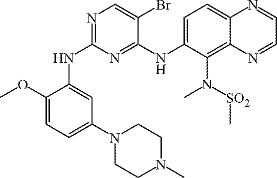 [SMILES: COc1ccc(N2CCN(C)CC2)cc1Nc1ncc(Br)c(Nc2ccc3nccnc3c2N(C)S(C)(=O)=O)n1] | MS (ESI) m/z: 628 [M+H]⁺. |
| I-76 | 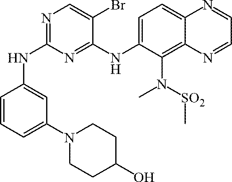 [SMILES: CN(c1c(Nc2nc(Nc3cccc(N4CCC(O)CC4)c3)ncc2Br)ccc2nccnc12)S(C)(=O)=O] | ¹H NMR (600 MHz, DMSO-d₆) δ 9.88 (s, 1H), 9.01 (d, J = 1.8 Hz, 1H), 8.96 (d, J = 1.9 Hz, 1H), 8.76 (s, 1H), 8.45 (s, 1H), 8.14 (d, J = 9.4 Hz, 1H), 7.82 - 7.76 (m, 1H), 7.60 - 7.51 (m, 1H), 7.35 (t, J = 8.1 Hz, 1H), 7.09 (d, J = 8.1 Hz, 1H), 3.78 - 3.66 (m, 1H), 3.56 - 3.48 (m, 2H), 3.43 (s, 3H), 3.25 (s, 3H), 3.23 - 3.20 (m, 2H), 1.95 - 1.84 (m, 2H), 1.72 - 1.61 (m, 2H). MS (ESI) m/z: 599 [M+H]⁺. |
| I-77 | 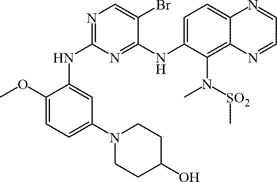 [SMILES: COc1ccc(N2CCC(O)CC2)cc1Nc1ncc(Br)c(Nc2ccc3nccnc3c2N(C)S(C)(=O)=O)n1] | 1H NMR (600 MHz, DMSO-d₆) δ 8.99 (d, J = 1.9 Hz, 1H), 8.94 (d, J = 1.9 Hz, 1H), 8.87 (d, J = 8.4 Hz, 1H), 8.75 - 8.69 (m, 2H), 8.41 (s, 1H), 8.04 (d, J = 9.3 Hz, 1H), 7.40 - 7.30 (m, 1H), 7.22 (d, J = 8.9 Hz, 1H), 3.86 (s, 3H), 3.77 - 3.62 (m, 1H), 3.51 - 3.44 (m, 2H), 3.42 (s, 3H), 3.34 - 3.27 (m, 2H), 3.25 (s, 3H), 1.95 - 1.82 (m, 2H), 1.75 - 1.62 (m, 2H). MS (ESI) m/z: 629 [M+H]⁺. |
| 1-78 | 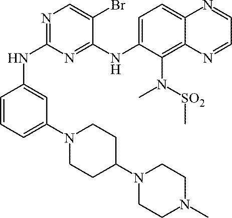 [SMILES: CN1CCN(C2CCN(c3cccc(Nc4ncc(Br)c(Nc5ccc6nccnc6c5N(C)S(C)(=O)=O)n4)c3)CC2)CC1] | ¹H NMR (600 MHz, DMSO-d₆) δ 9.53 (s, 1H), 9.00 (d, J = 1.8 Hz, 1H), 8.96 (d, J = 1.9 Hz, 1H), 8.69 (s, 1H), 8.41 (s, 1H), 8.11 (d, J = 9.3 Hz, 1H), 7.29 - 7.23 (m, 1H), 7.19 - 7.16 (m, 1H), 7.13 (t, J = 8.0 Hz, 1H), 6.68 (dd, J = 8.2, 2.3 Hz, 1H), 3.76 - 3.67 (m, 2H), 3.63 - 3.46 (m, 5H), 3.41 (s, 3H), 3.25 (s, 3H), 3.20 - 2.92 (m, 4H), 2.86 (s, 3H), 2.72 - 2.57 (m, 2H), 2.04 - 1.91 (m, 2H), 1.68 - 1.51 (m, 2H). MS (ESI) m/z: 681 [M+H]⁺. |
| I-79 | 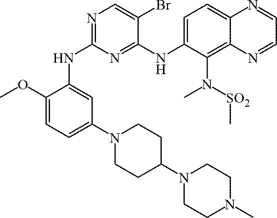 [SMILES: COc1ccc(N2CCC(N3CCN(C)CC3)CC2)cc1Nc1ncc(Br)c(Nc2ccc3nccnc3c2N(C)S(C)(=O)=O)n1] | MS (ESI) m/z: 711 [M+H]⁺. |
| I-80 | 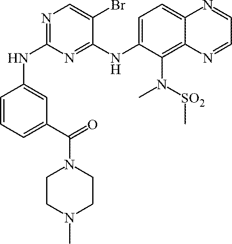 [SMILES: CN1CCN(C(=O)c2cccc(Nc3ncc(Br)c(Nc4ccc5nccnc5c4N(C)S(C)(=O)=O)n3)c2)CC1] | ¹H NMR (600 MHz, DMSO-d₆) δ 10.32 - 10.20 (m, 1H), 9.80 (s, 1H), 9.00 (d, J = 1.9 Hz, 1H), 8.96 (d, J = 1.8 Hz, 1H), 8.71 (s, 1H), 8.44 (s, 1H), 8.15 (d, J = 9.3 Hz, 1H), 7.82 (s, 1H), 7.70 (dd, J = 8.1, 2.1 Hz, 1H), 7.36 (t, J = 7.8 Hz, 1H), 7.07 (d, J = 7.5 Hz, 1H), 3.41 (s, 3H), 3.39 - 3.31 (m, 4H), 3.25 (s, 3H), 3.18 - 2.94 (m, 4H), 2.83 (s, 3H). MS (ESI) m/z: 626 [M+H]⁺. |
| I-81 | 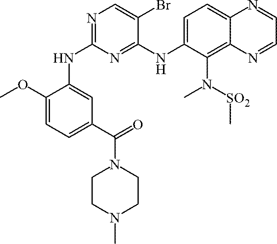 [SMILES: COc1ccc(C(=O)N2CCN(C)CC2)cc1Nc1ncc(Br)c(Nc2ccc3nccnc3c2N(C)S(C)(=O)=O)n1] | ¹H NMR (600 MHz, DMSO-d₆) δ 10.18 - 10.08 (m, 1H), 8.99 (d, J = 1.8 Hz, 1H), 8.94 (d, J = 1.8 Hz, 1H), 8.70 (s, 1H), 8.65 (s, 1H), 8.40 (s, 1H), 8.05 (d, J = 9.4 Hz, 1H), 7.94 (s, 1H), 7.27 (d, J = 8.4, 2.1 Hz, 1H), 7.18 (d, J = 8.5 Hz, 1H), 3.89 (s, 3H), 3.50 - 3.42 (m, 4H), 3.40 (s, 3H), 3.25 (s, 3H), 3.14 - 3.02 (m, 4H), 2.83 (s, 3H). MS (ESI) m/z: 656 [M+H]⁺. |
| I-82 | 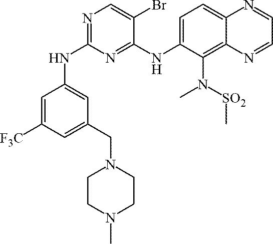 [SMILES: CN1CCN(Cc2cc(Nc3ncc(Br)c(Nc4ccc5nccnc5c4N(C)S(C)(=O)=O)n3)cc(C(F)(F)F)c2)CC1] | ¹H NMR (600 MHz, DMSO-d₆) δ 10.01 (s, 1H), 9.02 (d, J = 1.8 Hz, 1H), 8.98 (d, J = 1.9 Hz, 1H), 8.94 - 8.87 (m, 1H), 8.77 (s, 1H), 8.49 (s, 1H), 8.13 (d, J = 9.3 Hz, 1H), 8.06 (s, 1H), 7.88 (s, 1H), 7.32 (s, 1H), 3.83 (s, 2H), 3.42 (s, 3H), 3.25 (s, 3H), 3.21 - 2.94 (m, 4H), 2.82 (s, 3H), 2.76 - 2.57 (m, 4H). MS (ESI) m/z: 680 [M+H]⁺. |
| I-83 | 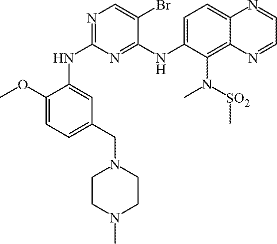 [SMILES: COc1ccc(CN2CCN(C)CC2)cc1Nc1ncc(Br)c(Nc2ccc3nccnc3c2N(C)S(C)(=O)=O)n1] | ¹H NMR (600 MHz, DMSO-d₆) δ 8.99 (d, J = 1.8 Hz, 1H), 8.94 (d, J = 1.8 Hz, 1H), 8.92 - 8.87 (m, 1H), 8.72 (s, 1H), 8.70 (s, 1H), 8.37 (s, 1H), 8.01 (d, J = 9.4 Hz, 1H), 7.76 (s, 1H), 7.24 - 7.20 (m, 1H), 7.16 (d, J = 8.4 Hz, 1H), 4.02 - 3.89 (m, 2H), 3.84 (s, 3H), 3.40 (s, 3H), 3.25 (s, 3H), 3.24 - 3.08 (m, 4H), 2.80 (s, 3H), 2.77 - 2.58 (m, 4H). MS (ESI) m/z: 642 [M+H]⁺. |
| I-84 | 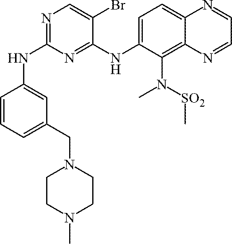 [SMILES: CN1CCN(Cc2cccc(Nc3ncc(Br)c(Nc4ccc5nccnc5c4N(C)S(C)(=O)=O)n3)c2)CC1] | ¹H NMR (600 MHz, DMSO-d₆) δ 9.76 (s, 1H), 9.03 (d, J = 10.4 Hz, 1H), 9.01 (d, J = 1.8 Hz, 1H), 8.96 (d, J = 1.8 Hz, 1H), 8.73 (s, 1H), 8.43 (s, 1H), 8.15 (d, J = 9.3 Hz, 1H), 7.70 (d, J = 8.3 Hz, 1H), 7.66 (s, 1H), 7.33 (t, J = 7.8 Hz, 1H), 7.07 (d, J = 7.5 Hz, 1H), 3.99 (s, 2H), 3.41 (s, 3H), 3.26 (s, 3H), 3.23 - 2.89 (m, 4H), 2.83 (s, 3H), 2.82 - 2.60 (m, 4H). MS (ESI) m/z: 612 [M+H]⁺. |
| I-85 | 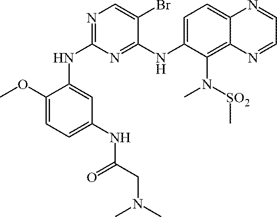 [SMILES: COc1ccc(NC(=O)CN(C)C)cc1Nc1ncc(Br)c(Nc2ccc3nccnc3c2N(C)S(C)(=O)=O)n1] | ¹H NMR (600 MHz, DMSO-d₆) δ 10.50 (s, 1H), 8.98 (d, J = 1.8 Hz, 1H), 8.95 (d, J = 9.4 Hz, 1H), 8.93 (d, J = 1.9 Hz, 1H), 8.69 (s, 1H), 8.65 (s, 1H), 8.36 (s, 1H), 7.96 (d, J = 9.4 Hz, 1H), 7.89 (s, 1H), 7.45 (d, J = 8.8, 2.6 Hz, 1H), 7.13 (d, J = 8.9 Hz, 1H), 4.10 (s, 2H), 3.81 (s, 3H), 3.40 (s, 3H), 3.25 (s, 3H), 2.86 (s, 6H). MS (ESI) m/z: 630 [M+H]⁺. |
| I-86 | 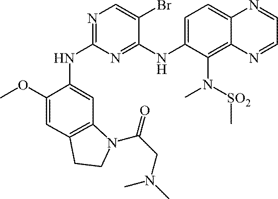 [SMILES: COc1cc2c(cc1Nc1ncc(Br)c(Nc3ccc4nccnc4c3N(C)S(C)(=O)=O)n1)N(C(=O)CN(C)C)CC2] | MS (ESI) m/z: 656 [M+H]⁺. |
| I-87 | 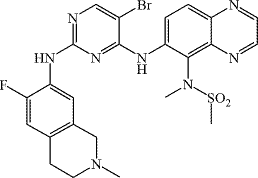 [SMILES: CN1CCc2cc(F)c(Nc3ncc(Br)c(Nc4ccc5nccnc5c4N(C)S(C)(=O)=O)n3)cc2C1] | ¹H NMR (600 MHz, DMSO-d₆) δ 10.33 - 10.20 (m, 1H), 9.37 (s, 1H), 8.99 (d, J = 1.8 Hz, 1H), 8.94 (d, J = 1.9 Hz, 1H), 8.71 - 8.67 (m, 1H), 8.38 (s, 1H), 8.10 (d, J = 9.6 Hz, 1H), 7.45 (d, J = 7.8 Hz, 1H), 7.29 (d, J = 11.2 Hz, 1H), 4.44 - 4.36 (m, 1H), 4.25 - 4.14 (m, 1H), 3.77 - 3.65 (m, 1H), 3.40 (s, 3H), 3.37 - 3.31 (m, 1H), 3.25 (s, 3H), 3.15 - 3.04 (m, 2H), 2.93 (s, 3H). MS (ESI) m/z: 587 [M+H]⁺. |
| I-88 | 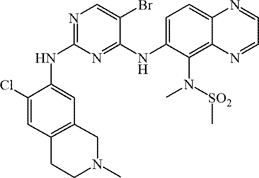 [SMILES: CN1CCc2cc(Cl)c(Nc3ncc(Br)c(Nc4ccc5nccnc5c4N(C)S(C)(=O)=O)n3)cc2C1] | ¹H NMR (600 MHz, DMSO-d₆) δ 9.20 (s, 1H), 8.99 (d, J = 1.8 Hz, 1H), 8.94 (d, J = 1.9 Hz, 1H), 8.88 (d, J = 9.4 Hz, 1H), 8.68 (s, 1H), 8.36 (s, 1H), 8.04 (d, J = 9.4 Hz, 1H), 7.54 (s, 1H), 7.46 (s, 1H), 4.48 - 4.37 (m, 1H), 4.28 - 4.16 (m, 1H), 3.77 - 3.66 (m, 1H), 3.40 (s, 3H), 3.38 - 3.31 (m, 1H), 3.25 (s, 3H), 3.21 - 3.08 (m, 2H), 2.94 (s, 3H). MS (ESI) m/z: 603 [M+H]⁺. |
| I-89 | 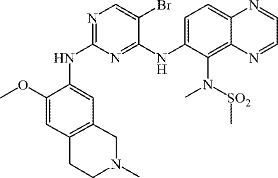 [SMILES: COc1cc2c(cc1Nc1ncc(Br)c(Nc3ccc4nccnc4c3N(C)S(C)(=O)=O)n1)CN(C)CC2] | ¹H NMR (600 MHz, DMSO-d₆) δ 9.00 (d, J = 1.8 Hz, 1H), 8.95 (d, J = 1.7 Hz, 1H), 8.89 - 8.79 (m, 1H), 8.78 - 8.73 (m, 1H), 8.59 (s, 1H), 8.38 (s, 1H), 8.19 - 8.12 (m, 1H), 7.51 (s, 1H), 6.99 (s, 1H), 4.26 - 4.07 (m, 1H), 4.05 - 3.94 (m, 1H), 3.81 (s, 3H), 3.72 - 3.63 (m, 1H), 3.39 (s, 3H), 3.31 (s, 1H), 3.23 (s, 3H), 3.20 - 3.09 (m, 1H), 3.07 - 2.99 (m, 1H), 2.89 (s, 3H). MS (ESI) m/z: 599 [M+H]⁺. |
| I-90 | 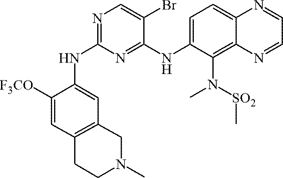 [SMILES: CN1CCc2cc(OC(F)(F)F)c(Nc3ncc(Br)c(Nc4ccc5nccnc5c4N(C)S(C)(=O)=O)n3)cc2C1] | ¹H NMR (600 MHz, DMSO-d₆) δ 9.33 (s, 1H), 9.00 (d, J = 1.9 Hz, 1H), 8.94 (d, J = 2.0 Hz, 1H), 8.88 (d, J = 9.5 Hz, 1H), 8.70 (s, 1H), 8.37 (s, 1H), 8.10 (d, J = 9.5 Hz, 1H), 7.54 (s, 1H), 7.43 (s, 1H), 4.47 - 4.37 (m, 1H), 4.30 - 4.14 (m, 1H), 3.78 - 3.65 (m, 2H), 3.40 (s, 3H), 3.25 (s, 3H), 3.21 - 3.12 (m, 2H), 2.94 (s, 3H). MS (ESI) m/z: 653 [M+H]⁺. |
| I-91 | 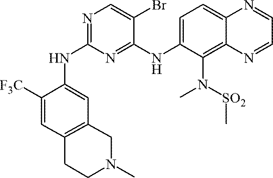 [SMILES: CN1CCc2cc(C(F)(F)F)c(Nc3ncc(Br)c(Nc4ccc5nccnc5c4N(C)S(C)(=O)=O)n3)cc2C1] | MS (ESI) m/z: 637 [M+H]⁺. |
| I-92 | 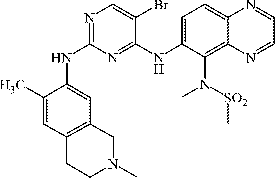 [SMILES: Cc1cc2c(cc1Nc1ncc(Br)c(Nc3ccc4nccnc4c3N(C)S(C)(=O)=O)n1)CN(C)CC2] | ¹H NMR (600 MHz, DMSO-d₆) δ 9.18 (s, 1H), 8.99 (d, J = 1.7 Hz, 1H), 8.94 (d, J = 1.8 Hz, 1H), 8.90 - 8.83 (m, 1H), 8.76 - 8.71 (m, 1H), 8.35 (s, 1H), 8.06 - 8.00 (m, 1H), 7.24 (s, 1H), 7.18 (s, 1H), 4.48 - 4.35 (m, 1H), 4.28 - 4.16 (m, 1H), 3.81 - 3.65 (m, 1H), 3.40 (s, 3H), 3.38 - 3.31 (m, 1H), 3.26 (s, 3H), 3.20 - 3.13 (m, 1H), 3.09 - 3.02 (m, 1H), 2.95 (s, 3H), 2.20 (s, 3H). MS (ESI) m/z: 583 [M+H]⁺. |
| I-93 | 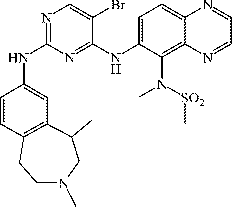 [SMILES: CC1CN(C)CCc2ccc(Nc3ncc(Br)c(Nc4ccc5nccnc5c4N(C)S(C)(=O)=O)n3)cc21] | ¹H NMR (600 MHz, DMSO-d₆) δ 9.48 (s, 1H), 9.07 - 8.90 (m, 3H), 8.70 - 8.57 (m, 1H), 8.38 (s, 1H), 8.09 (d, J = 9.4 Hz, 1H), 7.44 - 7.36 (m, 1H), 7.35 - 7.28 (m, 1H), 6.96 (d, J = 8.1 Hz, 1H), 3.39 (s, 3H), 3.24 (s, 3H), 2.91 - 2.76 (m, 3H), 2.42 - 2.28 (m, 2H), 2.24 (s, 3H), 2.19 - 2.04 (m, 2H), 1.15 - 1.04 (m, 3H). MS (ESI) m/z: 597 [M+H]⁺. |
| I-94 | 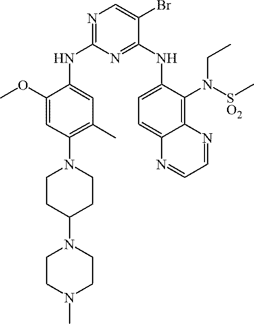 | ¹H NMR (600 MHz, Methanol-d₄) δ 8.91 (d, J = 1.9 Hz, 1H), 8.82 (d, J = 1.9 Hz, 1H), 8.80 (d, J = 8.7 Hz, 1H), 8.19 (s, 1H), 7.97 (d, J = 9.3 Hz, 1H), 7.59 (s, 1H), 6.74 (s, 1H), 4.17 - 4.06 (m, 1H), 3.84 - 3.75 (m, 4H), 3.26 - 3.18 (m, 4H), 3.16 (s, 3H), 3.13 - 3.00 (m, 3H), 2.86 - 2.74 (m, 6H), 2.65 (s, 3H), 2.09 - 2.03 (m, 5H), 1.88 - 1.75 (m, 2H), 1.02 (t, J = 7.2 Hz, 3H). MS (ESI) m/z: 739 [M+H]⁺. |
| I-95 | 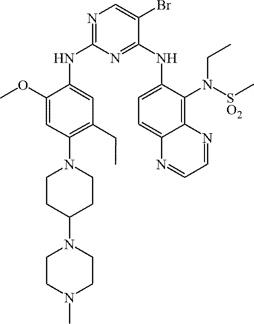 | ¹H NMR (600 MHz, Methanol-d₄) δ 8.96 - 8.90 (m, 1H), 8.90 - 8.81 (m, 1H), 8.68 - 8.58 (m, 1H), 8.24 (s, 1H), 8.00 (d, J = 9.4 Hz, 1H), 7.56 (s, 1H), 6.83 (s, 1H), 4.20 - 4.07 (m, 1H), 3.83 (s, 3H), 3.82 - 3.74 (m, 1H), 3.50 - 3.37 (m, 4H), 3.30 - 3.27 (m, 2H), 3.26 - 3.17 (m, 2H), 3.14 (s, 3H), 3.11 - 3.04 (m, 1H), 2.95 - 2.87 (m, 2H), 2.87 (s, 3H), 2.67 - 2.62 (m, 5H), 2.57 - 2.46 (m, 1H), 2.45 - 2.35 (m, 1H), 2.19 - 2.09 (m, 2H), 1.98 - 1.81 (m, 2H), 1.00 (t, J = 7.2 Hz, 3H). MS (ESI) m/z: 753 [M+H]⁺. |
| I-96 | 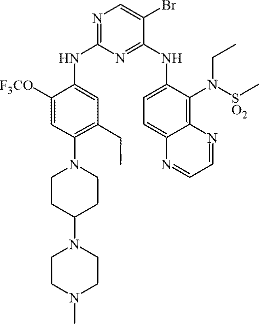 [SMILES: CCc1cc(Nc2ncc(Br)c(Nc3ccc4nccnc4c3N(CC)S(C)(=O)=O)n2)c(OC(F)(F)F)cc1N1CCC(N2CCN(C)CC2)CC1] | ¹H NMR (600 MHz, Methanol-d₄) δ 8.92 (d, J = 1.9 Hz, 1H), 8.84 (d, J = 1.8 Hz, 1H), 8.65 (d, J = 9.3 Hz, 1H), 8.24 (s, 1H), 7.88 (d, J = 9.3 Hz, 1H), 7.56 (s, 1H), 7.05 (s, 1H), 4.19 - 4.06 (m, 1H), 3.83 - 3.72 (m, 1H), 3.27 - 3.15 (m, 8H), 3.00 - 2.91 (m, 1H), 2.82 (s, 3H), 2.78 - 2.72 (m, 2H), 2.65 (s, 5H), 2.63 - 2.56 (m, 1H), 2.56 - 2.44 (m, 1H), 2.16 - 2.04 (m, 2H), 1.88 - 1.74 (m, 2H), 1.01 (t, J = 7.1 Hz, 3H), 0.92 (t, J = 7.5 Hz, 3H). MS (ESI) m/z: 807 [M+H]⁺. |
| I-97 | 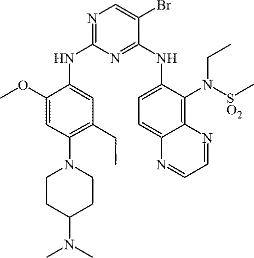 | ¹H NMR (600 MHz, Methanol-d₄) δ 8.93 (d, J = 1.8 Hz, 1H), 8.85 (d, J = 1.8 Hz, 1H), 8.72 - 8.64 (m, 1H), 8.21 (s, 1H), 7.98 (d, J = 9.3 Hz, 1H), 7.64 (s, 1H), 6.78 (s, 1H), 4.19 - 4.05 (m, 1H), 3.83 (s, 3H), 3.81 - 3.76 (m, 1H), 3.34 - 3.28 (m, 1H), 3.18 - 3.12 (m, 5H), 2.93 (s, 6H), 2.86 - 2.78 (m, 2H), 2.58 - 2.43 (m, 1H), 2.44 - 2.31 (m, 1H), 2.22 - 2.11 (m, 2H), 1.97 - 1.81 (m, 2H), 1.01 (t, J = 7.2 Hz, 3H), 0.84 - 0.71 (m, 3H). MS (ESI) m/z: 698 [M+H]⁺. |
| I-98 | 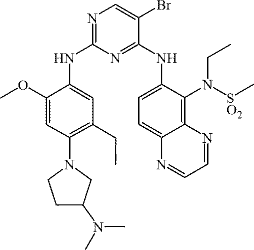 | ¹H NMR (600 MHz, Methanol-d₄) δ 8.93 (d, J = 1.8 Hz, 1H), 8.86 (d, J = 1.9 Hz, 1H), 8.71 - 8.58 (m, 1H), 8.22 (s, 1H), 7.98 (d, J = 9.2 Hz, 1H), 7.57 (d, J = 4.6 Hz, 1H), 6.80 (d, J = 1.4 Hz, 1H), 4.19 - 4.07 (m, 1H), 4.05 - 3.96 (m, 1H), 3.84 (s, 3H), 3.82 - 3.72 (m, 1H), 3.45 - 3.36 (m, 2H), 3.33 - 3.25 (m, 1H), 3.16 (s, 3H), 3.13 - 3.03 (m, 1H), 3.01 - 2.93 (m, 6H), 2.56 - 2.44 (m, 2H), 2.44 - 2.33 (m, 1H), 2.26 - 2.11 (m, 1H), 1.01 (t, J = 7.2 Hz, 3H), 0.90 - 0.71 (m, 3H). MS (ESI) m/z: 684 [M+H]⁺. |
| I-99 | 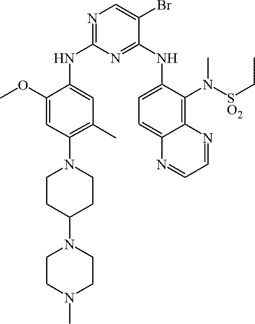 [SMILES: CCS(=O)(=O)N(C)c1c(Nc2nc(Nc3cc(C)c(N4CCC(N5CCN(C)CC5)CC4)cc3OC)ncc2Br)ccc2nccnc12] | ¹H NMR (600 MHz, MeOD) δ 8.99 (d, J = 1.8 Hz, 1H), 8.91 (d, J = 1.8 Hz, 1H), 8.62 (d, J = 9.2 Hz, 1H), 8.26 (s, 1H), 8.06 (d, J = 9.3 Hz, 1H), 7.45 (s, 1H), 6.83 (s, 1H), 3.85 (s, 3H), 3.52 - 3.48 (m, 2H), 3.47 (s, 3H), 3.41 - 3.32 (m, 8H), 3.24 - 3.19 (m, 2H), 3.09 - 3.01 (m, 1H), 2.95 - 2.90 (m, 2H), 2.88 (s, 3H), 2.19 - 2.13 (m, 2H), 2.05 (s, 3H), 1.96 - 1.85 (m, 2H), 1.42 (t, J = 7.4 Hz, 3H). MS (ESI) m/z: 739 [M+H]⁺. |
| I-100 | 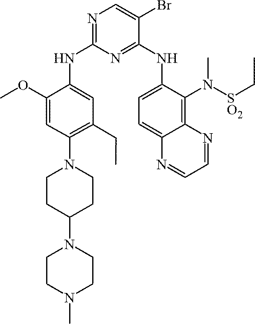 [SMILES: CCc1cc(Nc2ncc(Br)c(Nc3ccc4nccnc4c3N(C)S(=O)(=O)CC)n2)c(OC)cc1N1CCC(N2CCN(C)CC2)CC1] | ¹H NMR (600 MHz, MeOD) δ 8.94 (d, J =1.9 Hz, 1H), 8.88 (d, J = 1.8 Hz, 1H), 8.44 - 8.34 (m, 1H), 8.23 (s, 1H), 8.02 - 7.97 (m, 1H), 7.31 (s, 1H), 6.79 (s, 1H), 3.79 (s, 3H), 3.57 (s, 8H), 3.48 - 3.42 (m, 2H), 3.41 (s, 3H), 3.33 - 3.28 (m, 1H), 3.18 - 3.13 (m, 2H), 2.93 (s, 3H), 2.89 - 2.79 (m, 2H), 2.40 - 2.27 (m, 2H), 2.21 - 2.14 (m, 2H), 1.96 - 1.85 (m, 2H), 1.36 (t, J = 7.4 Hz, 3H), 0.87 - 0.69 (m, 3H). MS (ESI) m/z: 753 [M+H]⁺. |
| I-101 | 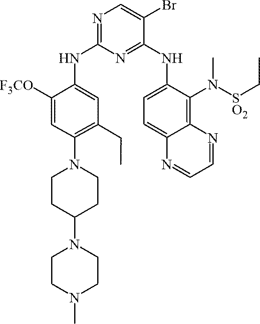 [SMILES: CCc1cc(Nc2ncc(Br)c(Nc3ccc4nccnc4c3N(C)S(=O)(=O)CC)n2)c(OC(F)(F)F)cc1N1CCC(N2CCN(C)CC2)CC1] | ¹H NMR (600 MHz, MeOD) δ 8.95 (d, J = 1.9 Hz, 1H), 8.87 - 8.86 (m, 1H), 8.47 (d, J = 9.8 Hz, 1H), 8.27 (s, 1H), 7.89 (d, J = 9.3 Hz, 1H), 7.49 (s, 1H), 7.05 (s, 1H), 3.55 - 3.46 (m, 8H), 3.43 (s, 3H), 3.25 - 3.10 (m, 5H), 2.92 (s, 3H), 2.82 - 2.72 (m, 2H), 2.49 (q, J = 7.5 Hz, 2H), 2.22 - 2.15 (m, 2H), 1.94 - 1.84 (m, 2H), 1.40 (t, J = 7.4 Hz, 3H), 0.89 (t, J = 7.5 Hz, 3H). MS (ESI) m/z: 807 [M+H]⁺. |
| I-102 | 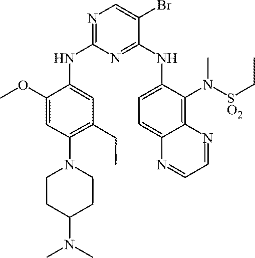 [SMILES: CCc1cc(Nc2ncc(Br)c(Nc3ccc4nccnc4c3N(C)S(=O)(=O)CC)n2)c(OC)cc1N1CCC(N(C)C)CC1] | ¹H NMR (600 MHz, MeOD) δ 8.98 (d, J = 1.8 Hz, 1H), 8.92 (d, J = 1.8 Hz, 1H), 8.54 - 8.44 (m, 1H), 8.26 (s, 1H), 8.02 (d, J = 9.1 Hz, 1H), 7.37 (s, 1H), 6.80 (s, 1H), 3.82 (s, 3H), 3.52 - 3.47 (m, 2H), 3.46 (s, 3H), 3.21 - 3.12 (m, 3H), 2.92 (s, 6H), 2.87 - 2.79 (m, 2H), 2.47 - 2.35 (m, 2H), 2.24 - 2.14 (m, 2H), 1.98 - 1.85 (m, 2H), 1.40 (t, J = 7.4 Hz, 3H), 0.95 - 0.73 (m, 3H). MS (ESI) m/z: 698 [M+H]⁺. |
| I-103 | 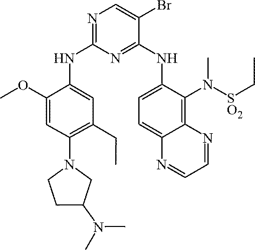 [SMILES: CCc1cc(Nc2ncc(Br)c(Nc3ccc4nccnc4c3N(C)S(=O)(=O)CC)n2)c(OC)cc1N1CCC(N(C)C)C1] | MS (ESI) m/z: 684 [M+H]⁺. |
| I-104 | 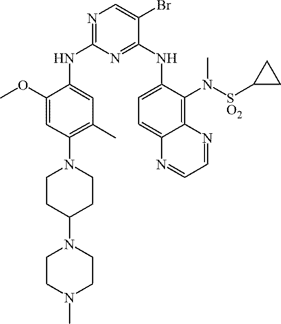 [SMILES: COc1cc(N2CCC(N3CCN(C)CC3)CC2)c(C)cc1Nc1ncc(Br)c(Nc2ccc3nccnc3c2N(C)S(=O)(=O)C2CC2)n1] | MS (ESI) m/z: 751 [M+H]⁺. |
| I-105 | 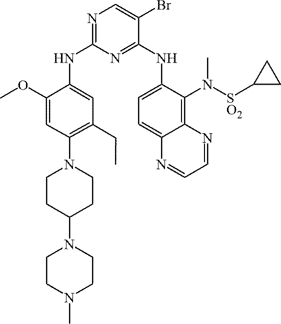 [SMILES: CCc1cc(Nc2ncc(Br)c(Nc3ccc4nccnc4c3N(C)S(=O)(=O)C3CC3)n2)c(OC)cc1N1CCC(N2CCN(C)CC2)CC1] | ¹H NMR (600 MHz, Methanol-d₄) δ 8.97 (d, J = 1.9 Hz, 1H), 8.89 (d, J = 1.8 Hz, 1H), 8.58 - 8.44 (m, 1H), 8.24 (s, 1H), 8.02 (d, J = 9.2 Hz, 1H), 7.41 (s, 1H), 6.83 (s, 1H), 3.83 (s, 3H), 3.59 - 3.36 (m, 11H), 3.27 - 3.13 (m, 3H), 2.94 - 2.87 (m, 5H), 2.86 - 2.82 (m, 1H), 2.50 - 2.36 (m, 2H), 2.22 - 2.09 (m, 2H), 1.98 - 1.83 (m, 2H), 1.21 - 1.09 (m, 2H), 0.92 - 0.76 (m, 3H), 0.72 - 0.67 (m, 1H), 0.67 - 0.59 (m, 1H). MS (ESI) m/z: 765 [M+H]⁺. |
| I-106 | 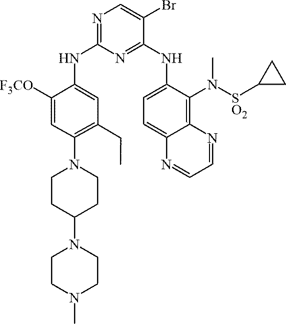 [SMILES: CCc1cc(Nc2ncc(Br)c(Nc3ccc4nccnc4c3N(C)S(=O)(=O)C3CC3)n2)c(OC(F)(F)F)cc1N1CCC(N2CCN(C)CC2)CC1] | ¹H NMR (600 MHz, MeOD) δ 8.92 (d, J = 1.9 Hz, 1H), 8.82 (d, J = 1.9 Hz, 1H), 8.52 (d, J = 9.2 Hz, 1H), 8.23 (s, 1H), 7.86 (d, J = 9.3 Hz, 1H), 7.48 (s, 1H), 7.03 (s, 1H), 3.45 (s, 3H), 3.43 - 3.30 (m, 8H), 3.18 - 3.11 (m, 2H), 3.10 - 3.03 (m, 1H), 2.85 (s, 3H), 2.79 - 2.69 (m, 2H), 2.55 - 2.45 (m, 2H), 2.19 - 2.07 (m, 2H), 1.92 - 1.79 (m, 2H), 1.29 - 1.19 (m, 1H), 1.18 - 1.10 (m, 2H), 0.91 (t, J = 7.5 Hz, 3H), 0.74 - 0.60 (m, 2H). MS (ESI) m/z: 819 [M+H]⁺. |
| I-107 | 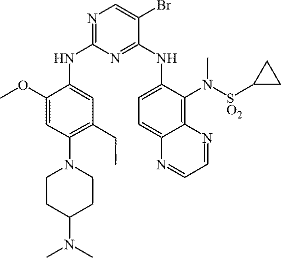 [SMILES: CCc1cc(Nc2ncc(Br)c(Nc3ccc4nccnc4c3N(C)S(=O)(=O)C3CC3)n2)c(OC)cc1N1CCC(N(C)C)CC1] | ¹H NMR (600 MHz, Methanol-d₄) δ 8.97 (d, J = 1.8 Hz, 1H), 8.89 (d, J = 1.9 Hz, 1H), 8.55 - 8.41 (m, 1H), 8.25 (d, J = 5.3 Hz, 1H), 8.00 (d, J = 9.2 Hz, 1H), 7.35 (s, 1H), 6.79 (s, 1H), 3.81 (s, 3H), 3.49 (s, 3H), 3.35 - 3.30 (m, 1H), 3.23 - 3.10 (m, 2H), 2.92 (s, 6H), 2.87 - 2.74 (m, 3H), 2.52 - 2.29 (m, 2H), 2.26 - 2.14 (m, 2H), 1.98 - 1.83 (m, 2H), 1.22 - 1.10 (m, 2H), 0.96 - 0.78 (m, 3H), 0.74 - 0.68 (m, 1H), 0.66 - 0.61 (m, 1H). MS (ESI) m/z: 710 [M+H]⁺. |
| I-108 | 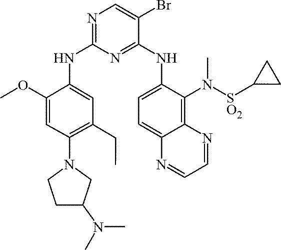 [SMILES: CCc1cc(Nc2ncc(Br)c(Nc3ccc4nccnc4c3N(C)S(=O)(=O)C3CC3)n2)c(OC)cc1N1CCC(N(C)C)C1] | ¹H NMR (600 MHz, MeOD) δ 8.93 (d, J = 1.8 Hz, 1H), 8.85 (d, J = 1.9 Hz, 1H), 8.52 - 8.33 (m, 1H), 8.20 (s, 1H), 8.01 - 7.89 (m, 1H), 7.25 (s, 1H), 6.76 - 6.71 (m, 1H), 4.02 - 3.93 (m, 1H), 3.78 (s, 3H), 3.45 (s, 3H), 3.40 - 3.35 (m, 2H), 3.14 - 3.03 (m, 1H), 2.92 (s, 6H), 2.84 - 2.76 (m, 1H), 2.47 - 2.42 (m, 1H), 2.41 - 2.32 (m, 2H), 2.24 - 2.08 (m, 1H), 1.30 - 1.03 (m, 3H), 0.93 - 0.71 (m, 3H), 0.69 - 0.56 (m, 2H). MS (ESI) m/z: 696 [M+H]⁺. |
| I-109 | 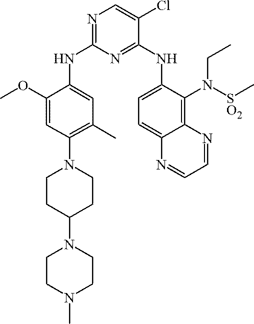 | MS (ESI) m/z: 695 [M+H]⁺. |
| I-110 | 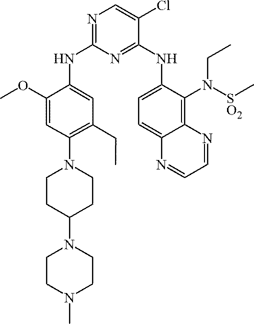 | ¹H NMR (600 MHz, Methanol-d₄) δ 8.90 (d, J = 1.9 Hz, 1H), 8.84 (d, J = 1.9 Hz, 1H), 8.59 - 8.46 (m, 1H), 8.14 (s, 1H), 7.96 (d, J = 9.3 Hz, 1H), 7.47 (s, 1H), 6.80 (s, 1H), 4.13 - 4.00 (m, 1H), 3.79 (s, 3H), 3.77 - 3.68 (m, 1H), 3.56 - 3.47 (m, 6H), 3.26 - 3.24 (m, 1H), 3.23 - 3.16 (m, 2H), 3.10 (s, 3H), 2.94 - 2.85 (m, 5H), 2.52 - 2.42 (m, 1H), 2.42 - 2.32 (m, 1H), 2.22 - 2.06 (m, 2H), 1.99 - 1.82 (m, 2H), 0.94 (t, J = 7.2 Hz, 3H), 0.86 - 0.71 (m, 3H). MS (ESI) m/z: 709 [M+H]⁺. |
| I-111 | 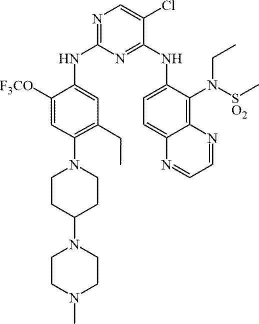 [SMILES: CCc1cc(Nc2ncc(Cl)c(Nc3ccc4nccnc4c3N(CC)S(C)(=O)=O)n2)c(OC(F)(F)F)cc1N1CCC(N2CCN(C)CC2)CC1] | ¹H NMR (600 MHz, Methanol-d₄) δ 8.89 (d, J = 1.9 Hz, 1H), 8.83 (d, J = 1.9 Hz, 1H), 8.53 - 8.41 (m, 1H), 8.18 (s, 1H), 7.86 (d, J = 9.4 Hz, 1H), 7.48 (s, 1H), 7.06 - 6.98 (m, 1H), 4.14 - 4.00 (m, 1H), 3.79 - 3.71 (m, 1H), 3.68 - 3.51 (m, 8H), 3.28 - 3.24 (m, 1H), 3.19 - 3.13 (m, 2H), 3.10 (s, 3H), 2.94 (s, 3H), 2.75 (t, J = 11.6 Hz, 2H), 2.62 - 2.45 (m, 2H), 2.30 - 2.12 (m, 2H), 1.98 - 1.82 (m, 2H), 0.97 - 0.88 (m, 6H). MS (ESI) m/z: 763 [M+H]⁺. |
| I-112 | 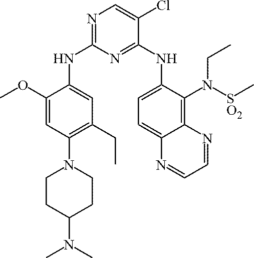 | ¹H NMR (600 MHz, Methanol-d₄) δ 8.92 (d, J = 1.9 Hz, 1H), 8.86 (d, J = 1.9 Hz, 1H), 8.66 - 8.50 (m, 1H), 8.15 (s, 1H), 7.97 (d, J = 9.4 Hz, 1H), 7.48 (s, 1H), 6.78 (s, 1H), 4.16 - 4.04 (m, 1H), 3.80 (s, 3H), 3.78 - 3.73 (m, 1H), 3.34 - 3.30 (m, 2H), 3.29 - 3.25 (m, 1H), 3.20 - 3.14 (m, 2H), 3.13 (s, 3H), 2.91 (s, 6H), 2.87 - 2.74 (m, 2H), 2.56 - 2.45 (m, 1H), 2.45 - 2.34 (m, 1H), 2.19 - 2.09 (m, 2H), 1.98 - 1.80 (m, 2H), 0.97 (t, J = 7.2 Hz, 3H), 0.90 - 0.72 (m, 3H). MS (ESI) m/z: 654 [M+H]⁺. |
| I-113 | 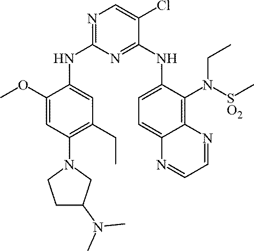 | ¹H NMR (600 MHz, Methanol-d₄) δ 8.92 (d, J = 1.9 Hz, 1H), 8.86 (d, J = 1.9 Hz, 1H), 8.65 - 8.41 (m, 1H), 8.17 (s, 1H), 8.03 - 7.90 (m, 1H), 7.34 (d, J = 4.2 Hz, 1H), 6.77 (d, J = 2.3 Hz, 1H), 4.17 - 4.04 (m, 1H), 4.05 - 3.94 (m, 1H), 3.79 (s, 3H), 3.79 - 3.70 (m, 1H), 3.52 - 3.38 (m, 2H), 3.29 - 3.24 (m, 1H), 3.16 - 3.08 (m, 4H), 3.00 - 2.90 (m, 6H), 2.56 - 2.34 (m, 3H), 2.27 - 2.10 (m, 1H), 1.05 - 0.94 (m, 3H), 0.85 (s, 3H). MS (ESI) m/z: 640 [M+H]⁺. |
| I-114 | 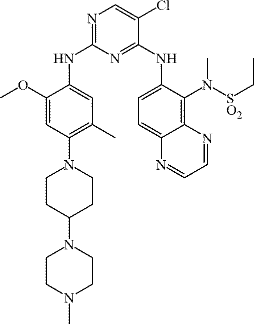 [SMILES: CCS(=O)(=O)N(C)c1c(Nc2nc(Nc3cc(C)c(N4CCC(N5CCN(C)CC5)CC4)cc3OC)ncc2Cl)ccc2nccnc12] | ¹H NMR (600 MHz, Methanol-d₄) δ 8.94 (d, J = 1.8 Hz, 1H), 8.84 (d, J = 1.9 Hz, 1H), 8.74 (d, J = 9.4 Hz, 1H), 8.11 (s, 1H), 7.99 (d, J = 9.3 Hz, 1H), 7.58 (s, 1H), 6.75 (s, 1H), 3.83 (s, 3H), 3.52 - 3.47 (m, 1H), 3.45 (s, 3H), 3.31 - 2.97 (m, 10H), 2.94 - 2.83 (m, 2H), 2.84 - 2.71 (m, 5H), 2.16 - 2.05 (m, 2H), 2.04 (s, 3H), 1.88 - 1.78 (m, 2H), 1.42 (t, J = 7.4 Hz, 3H). MS (ESI) m/z: 695 [M+H]⁺. |
| I-115 | 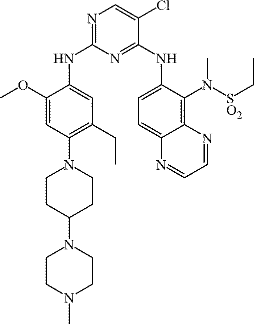 [SMILES: CCc1cc(Nc2ncc(Cl)c(Nc3ccc4nccnc4c3N(C)S(=O)(=O)CC)n2)c(OC)cc1N1CCC(N2CCN(C)CC2)CC1] | ¹H NMR (600 MHz, Methanol-d₄) δ 8.97 (d, J = 1.9 Hz, 1H), 8.88 (d, J = 1.9 Hz, 1H), 8.64 (d, J = 9.3 Hz, 1H), 8.16 (s, 1H), 8.02 (d, J = 9.3 Hz, 1H), 7.60 (s, 1H), 6.80 (s, 1H), 3.84 (s, 3H), 3.53 - 3.47 (m, 1H), 3.46 (s, 3H), 3.28 - 2.94 (m, 10H), 2.92 - 2.79 (m, 4H), 2.78 (s, 3H), 2.50 - 2.31 (m, 2H), 2.15 - 1.97 (m, 2H), 1.89 - 1.69 (m, 2H), 1.42 (t, J = 7.4 Hz, 3H), 0.90 - 0.68 (m, 3H). MS (ESI) m/z: 709 [M+H]⁺. |
| I-116 | 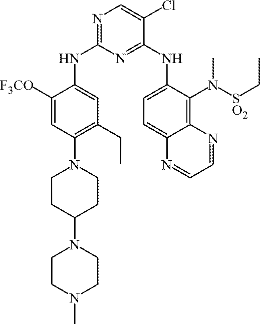 [SMILES: CCc1cc(Nc2ncc(Cl)c(Nc3ccc4nccnc4c3N(C)S(=O)(=O)CC)n2)c(OC(F)(F)F)cc1N1CCC(N2CCN(C)CC2)CC1] | ¹H NMR (600 MHz, Methanol-d₄) δ 8.95 (d, J = 1.9 Hz, 1H), 8.85 (d, J = 1.8 Hz, 1H), 8.68 (d, J = 9.4 Hz, 1H), 8.17 (s, 1H), 7.91 (d, J = 9.4 Hz, 1H), 7.59 (s, 1H), 7.05 (s, 1H), 3.54 - 3.48 (m, 1H), 3.45 (s, 3H), 3.25 - 3.00 (m, 10H), 2.93 - 2.82 (m, 2H), 2.79 (s, 3H), 2.76 - 2.70 (m, 2H), 2.59 - 2.47 (m, 2H), 2.13 - 2.02 (m, 2H), 1.85 - 1.71 (m, 2H), 1.42 (t, J = 7.4 Hz, 3H), 0.93 (t, J = 7.5 Hz, 3H). MS (ESI) m/z: 763 [M+H]⁺. |
| I-117 | 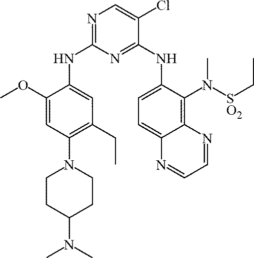 [SMILES: CCc1cc(Nc2ncc(Cl)c(Nc3ccc4nccnc4c3N(C)S(=O)(=O)CC)n2)c(OC)cc1N1CCC(N(C)C)CC1] | ¹H NMR (600 MHz, MeOD) δ 8.96 (d, J = 1.9 Hz, 1H), 8.89 (d, J = 1.9 Hz, 1H), 8.48 - 8.38 (m, 1H), 8.17 (s, 1H), 8.00 (d, J = 9.3 Hz, 1H), 7.41 (s, 1H), 6.79 (s, 1H), 3.82 (s, 3H), 3.56 (d, J = 12.1 Hz, 2H), 3.43 (s, 4H), 3.31 (s, 1H), 3.29 - 3.24 (m, J = 4.2, 3.4 Hz, 2H), 3.19 - 3.07 (m, 6H), 2.95 (s, 3H), 2.35 (s, 2H), 1.37 (t, J = 7.4 Hz, 3H), 0.76 (s, 3H). MS (ESI) m/z: 654 [M+H]⁺. |
| I-118 | 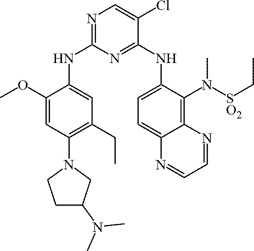 [SMILES: CCc1cc(Nc2ncc(Cl)c(Nc3ccc4nccnc4c3N(C)S(=O)(=O)CC)n2)c(OC)cc1N1CCC(N(C)C)C1] | ¹H NMR (600 MHz, MeOD) δ 8.96 (d, J = 1.8 Hz, 1H), 8.90 (d, J = 1.8 Hz, 1H), 8.51 - 8.32 (m, 1H), 8.17 (s, 1H), 8.05 - 7.93 (m, 1H), 7.28 (s, 1H), 6.79 - 6.74 (m, 1H), 4.03 - 3.94 (m, 1H), 3.81 (s, 3H), 3.49 - 3.45 (m, 1H), 3.43 (s, 3H), 3.41 - 3.36 (m, 2H), 3.32 - 3.30 (m, 1H), 3.14 (dd, J = 14.3, 7.3 Hz, 1H), 3.10 - 3.05 (m, 1H), 2.94 (s, 6H), 2.50 - 2.41 (m, 1H), 2.36 (s, 2H), 2.23 - 2.10 (m, 1H), 1.37 (t, J = 7.4 Hz, 3H), 1.06 - 0.66 (m, 3H). MS (ESI) m/z: 640 [M+H]⁺. |
| I-119 | 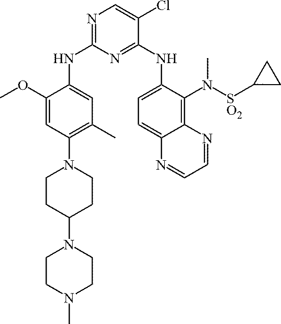 [SMILES: COc1cc(N2CCC(N3CCN(C)CC3)CC2)c(C)cc1Nc1ncc(Cl)c(Nc2ccc3nccnc3c2N(C)S(=O)(=O)C2CC2)n1] | MS (ESI) m/z: 707 [M+H]⁺. |
| I-120 | 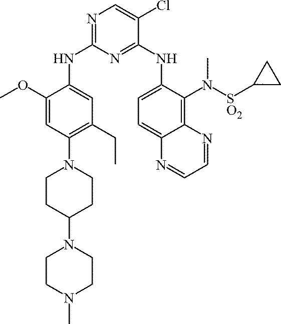 [SMILES: CCc1cc(Nc2ncc(Cl)c(Nc3ccc4nccnc4c3N(C)S(=O)(=O)C3CC3)n2)c(OC)cc1N1CCC(N2CCN(C)CC2)CC1] | ¹H NMR (600 MHz, MeOD) δ 8.97 (d, J = 1.8 Hz, 1H), 8.90 (d, J = 1.9 Hz, 1H), 8.51 - 8.42 (m, 1H), 8.17 (s, 1H), 8.04 (d, J = 9.3 Hz, 1H), 7.41 (s, 1H), 6.82 (s, 1H), 3.82 (s, 3H), 3.51 - 3.44 (m, 8H), 3.47 (s, 3H), 3.21 - 3.16 (m, 2H), 2.91 (s, 3H), 2.90 - 2.86 (m, 2H), 2.85 - 2.80 (m, 1H), 2.47 - 2.31 (m, 2H), 2.17 (d, J = 11.6 Hz, 2H), 1.97 - 1.85 (m, 2H), 1.27 - 1.12 (m, 3H), 0.88 - 0.75 (m, 3H), 0.72 - 0.62 (m, 2H). MS (ESI) m/z: 721 [M+H]⁺. |
| I-121 | 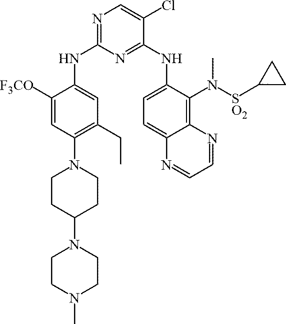 [SMILES: CCc1cc(Nc2ncc(Cl)c(Nc3ccc4nccnc4c3N(C)S(=O)(=O)C3CC3)n2)c(OC(F)(F)F)cc1N1CCC(N2CCN(C)CC2)CC1] | ¹H NMR (600 MHz, MeOD) δ 8.95 (d, J = 1.9 Hz, 1H), 8.85 (d, J = 2.0 Hz, 1H), 8.63 (d, J = 9.6 Hz, 1H), 8.18 (s, 1H), 7.91 (d, J = 9.3 Hz, 1H), 7.55 (s, 1H), 7.07 (s, 1H), 3.48 (s, 3H), 3.28 - 3.13 (m, 8H), 2.97 - 2.92 (m, 1H), 2.92 - 2.86 (m, 2H), 2.82 (s, 3H), 2.80 - 2.71 (m, 2H), 2.58 - 2.52 (m, 2H), 2.14 - 2.08 (m, 2H), 1.86 - 1.79 (m, 2H), 1.33 - 1.27 (m, 1H), 1.22 - 1.14 (m, 2H), 0.95 (t, J = 7.5 Hz, 3H), 0.75 - 0.64 (m, 2H). MS (ESI) m/z: 775 [M+H]⁺. |
| I-122 | 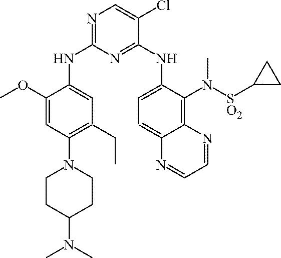 [SMILES: CCc1cc(Nc2ncc(Cl)c(Nc3ccc4nccnc4c3N(C)S(=O)(=O)C3CC3)n2)c(OC)cc1N1CCC(N(C)C)CC1] | ¹H NMR (600 MHz, MeOD) δ 8.98 (d, J =1.8 Hz, 1H), 8.89 (d, J = 1.9 Hz, 1H), 8.63 - 8.51 (m, 1H), 8.17 (s, 1H), 8.03 (d, J = 9.4 Hz, 1H), 7.47 (s, 1H), 6.80 (s, 1H), 3.84 (s, 3H), 3.50 (s, 3H), 3.16 (t, J = 13.8 Hz, 2H), 2.93 (s, 6H), 2.89 - 2.85 (m, 1H), 2.85 - 2.79 (m, 2H), 2.49 - 2.36 (m, 2H), 2.24 - 2.12 (m, 2H), 1.90 (q, J = 11.8, 4.1 Hz, 2H), 1.37 - 1.23 (m, 1H), 1.22 - 1.12 (m, 2H), 0.94 - 0.78 (m, 3H), 0.77 - 0.63 (m, 2H). MS (ESI) m/z: 666 [M+H]⁺. |
| I-123 | 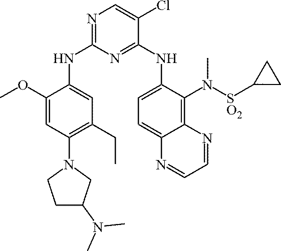 [SMILES: CCc1cc(Nc2ncc(Cl)c(Nc3ccc4nccnc4c3N(C)S(=O)(=O)C3CC3)n2)c(OC)cc1N1CCC(N(C)C)C1] | ¹H NMR (600 MHz, MeOD) δ 8.95 (d, J = 1.8 Hz, 1H), 8.87 (d, J = 1.8 Hz, 1H), 8.55 - 8.40 (m, 1H), 8.14 (s, 1H), 7.99 (d, J = 9.4 Hz, 1H), 7.35 (s, 1H), 6.81 - 6.73 (m, 1H), 4.03 - 3.93 (m, 1H), 3.81 (s, 3H), 3.46 (s, 3H), 3.40 - 3.34 (m, 2H), 3.12 - 3.02 (m, 1H), 2.93 (s, 6H), 2.86 - 2.77 (m, 1H), 2.52 - 2.43 (m, 1H), 2.44 - 2.34 (m, 2H), 2.21 - 2.12 (m, 1H), 1.26 - 1.08 (m, 3H), 0.90 - 0.77 (m, 3H), 0.72 - 0.59 (m, 2H). MS (ESI) m/z: 652 [M+H]⁺. |
| I-124 | 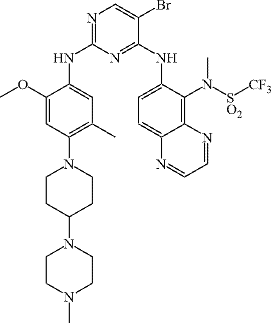 [SMILES: COc1cc(N2CCC(N3CCN(C)CC3)CC2)c(C)cc1Nc1ncc(Br)c(Nc2ccc3nccnc3c2N(C)S(=O)(=O)C(F)(F)F)n1] | ¹H NMR (600 MHz, Methanol-d₄) δ 8.99 (d, J = 1.9 Hz, 1H), 8.96 (d, J = 1.8 Hz, 1H), 8.25 (s, 1H), 8.20 - 8.06 (m, 2H), 7.13 (s, 1H), 6.68 (s, 1H), 3.77 (s, 3H), 3.61 - 3.42 (m, 11H), 3.23 - 3.18 (m, 1H), 3.19 - 3.09 (m, 2H), 2.91 (s, 3H), 2.84 - 2.73 (m, 2H), 2.21 - 2.08 (m, 2H), 1.95 - 1.81 (m, 2H), 1.65 (br s, 3H). MS (ESI) m/z: 779 [M+H]⁺. |
| I-125 | 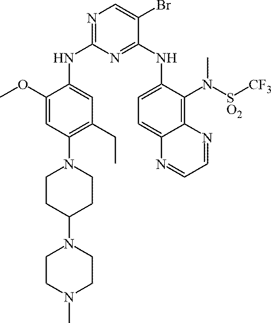 [SMILES: CCc1cc(Nc2ncc(Br)c(Nc3ccc4nccnc4c3N(C)S(=O)(=O)C(F)(F)F)n2)c(OC)cc1N1CCC(N2CCN(C)CC2)CC1] | ¹H NMR (600 MHz, Methanol-d₄) δ 9.08 - 8.98 (m, 1H), 8.98 - 8.92 (m, 1H), 8.33 (s, 1H), 8.28 - 8.24 (m, 1H), 8.13 (d, J = 10.6 Hz, 1H), 7.42 (s, 1H), 6.84 - 6.71 (m, 1H), 4.01 - 3.85 (m, 1H), 3.82 (s, 3H), 3.67 - 3.61 (m, 1H), 3.57 (s, 3H), 3.30 - 3.03 (m, 9H), 2.98 - 2.87 (m, 2H), 2.85 - 2.80 (m, 3H), 2.33 - 2.21 (m, 1H), 2.12 - 2.05 (m, 2H), 1.91 - 1.72 (m, 2H), 1.36 - 1.23 (m, 1H), 0.80 - 0.57 (m, 3H). MS (ESI) m/z: 793 [M+H]⁺. |
| I-126 | 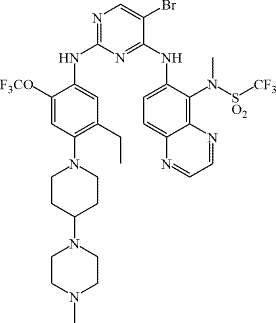 [SMILES: CCc1cc(Nc2ncc(Br)c(Nc3ccc4nccnc4c3N(C)S(=O)(=O)C(F)(F)F)n2)c(OC(F)(F)F)cc1N1CCC(N2CCN(C)CC2)CC1] | ¹H NMR (600 MHz, MeOD) δ 8.96 (d, J = 1.8 Hz, 1H), 8.93 (d, J = 1.9 Hz, 1H), 8.28 (s, 1H), 8.13 (d, J = 9.3 Hz, 1H), 8.01 (d, J = 9.2 Hz, 1H), 7.31 (s, 1H), 6.93 (s, 1H), 3.65 - 3.52 (m, 8H), 3.50 (s, 3H), 3.25 - 3.22 (m, 1H), 3.08 - 2.97 (m, 2H), 2.92 (s, 3H), 2.72 - 2.59 (m, 2H), 2.38 - 2.27 (m, 1H), 2.20 - 2.13 (m, 2H), 2.14 - 2.07 (m, 1H), 1.92 - 1.76 (m, 2H), 0.80 - 0.68 (m, 3H). MS (ESI) m/z: 847 [M+H]⁺. |
| I-127 | 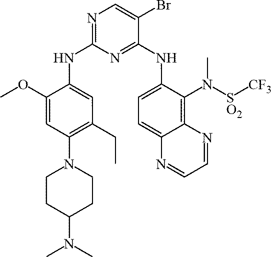 [SMILES: CCc1cc(Nc2ncc(Br)c(Nc3ccc4nccnc4c3N(C)S(=O)(=O)C(F)(F)F)n2)c(OC)cc1N1CCC(N(C)C)CC1] | ¹H NMR (600 MHz, Methanol-d₄) δ 9.02 (d, J = 1.8 Hz, 1H), 8.98 (d, J = 1.8 Hz, 1H), 8.28 (s, 1H), 8.25 - 8.11 (m, 2H), 7.24 (s, 1H), 6.72 (s, 1H), 3.80 (s, 3H), 3.56 (s, 3H), 3.17 - 3.00 (m, 3H), 2.92 (s, 6H), 2.83 - 2.70 (m, 2H), 2.35 - 2.23 (m, 1H), 2.21 - 2.11 (m, 2H), 2.10 - 1.95 (m, 1H), 1.92 - 1.80 (m, 2H), 0.88 - 0.51 (m, 3H). MS (ESI) m/z: 738 [M+H]⁺. |
| I-128 | 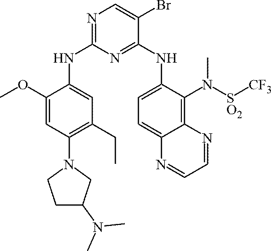 [SMILES: CCc1cc(Nc2ncc(Br)c(Nc3ccc4nccnc4c3N(C)S(=O)(=O)C(F)(F)F)n2)c(OC)cc1N1CCC(N(C)C)C1] | ¹H NMR (600 MHz, Methanol-d₄) δ 9.00 (d, J = 1.9 Hz, 1H), 8.93 (d, J = 1.9 Hz, 1H), 8.42 (d, J = 9.3 Hz, 1H), 8.26 (s, 1H), 7.87 (d, J = 8.6 Hz, 1H), 7.46 (s, 1H), 6.75 (s, 1H), 3.83 (s, 3H), 3.58 (s, 3H), 3.07 - 3.03 (m, 1H), 2.96 (d, J = 2.4 Hz, 6H), 2.53 - 2.39 (m, 1H), 2.28 (s, 1H), 2.21 - 2.10 (m, 2H), 2.04 - 1.97 (m, 1H), 1.82 - 1.74 (m, 1H), 1.68 - 1.51 (m, 2H), 0.76 - 0.59 (m, 3H). MS (ESI) m/z: 724 [M+H]⁺. |
| I-129 | 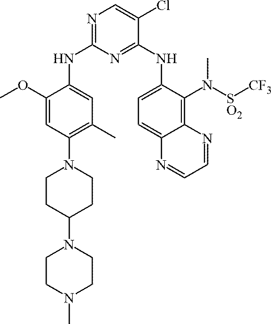 [SMILES: COc1cc(N2CCC(N3CCN(C)CC3)CC2)c(C)cc1Nc1ncc(Cl)c(Nc2ccc3nccnc3c2N(C)S(=O)(=O)C(F)(F)F)n1] | ¹H NMR (600 MHz, MeOD) δ 9.01 (d, J = 1.8 Hz, 1H), 8.97 (d, J = 1.8 Hz, 1H), 8.18 (s, 1H), 8.17 - 8.15 (m, 1H), 7.20 (s, 1H), 6.71 (s, 1H), 3.79 (s, 3H), 3.53 (s, 3H), 3.49 - 3.31 (m, 8H), 3.21 - 3.13 (m, 2H), 3.13 - 3.06 (m, 1H), 2.89 (s, 3H), 2.86 - 2.78 (m, 2H), 2.17 - 2.08 (m, 2H), 1.92 - 1.82 (m, 2H), 1.66 (s, 3H). MS (ESI) m/z: 735 [M+H]⁺. |
| I-130 | 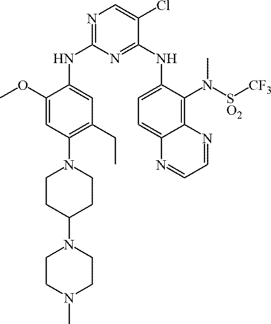 [SMILES: CCc1cc(Nc2ncc(Cl)c(Nc3ccc4nccnc4c3N(C)S(=O)(=O)C(F)(F)F)n2)c(OC)cc1N1CCC(N2CCN(C)CC2)CC1] | ¹H NMR (600 MHz, Methanol-d₄) δ 9.01 (d, J = 1.9 Hz, 1H), 8.98 (d, J = 1.8 Hz, 1H), 8.19 (s, 1H), 8.18 - 8.04 (m, 2H), 7.25 (s, 1H), 6.74 (s, 1H), 3.79 (s, 3H), 3.54 (s, 3H), 3.53 - 3.32 (m, 8H), 3.16 - 3.04 (m, 3H), 2.89 (s, 3H), 2.86 - 2.77 (m, 2H), 2.29 - 2.18 (m, 1H), 2.17 - 2.08 (m, 2H), 2.04 - 1.91 (m, 1H), 1.89 - 1.78 (m, 2H), 0.82 - 0.28 (m, 2H). MS (ESI) m/z: 749 [M+H]⁺. |
| I-131 | 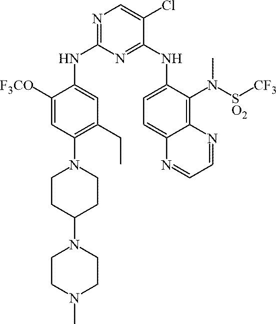 [SMILES: CCc1cc(Nc2ncc(Cl)c(Nc3ccc4nccnc4c3N(C)S(=O)(=O)C(F)(F)F)n2)c(OC(F)(F)F)cc1N1CCC(N2CCN(C)CC2)CC1] | ¹H NMR (600 MHz, Methanol-d₄) δ 8.96 (d, J = 1.8 Hz, 1H), 8.93 (d, J = 1.9 Hz, 1H), 8.20 (s, 1H), 8.09 (d, J = 9.2 Hz, 1H), 8.03 (d, J = 9.2 Hz, 1H), 7.30 (s, 1H), 6.92 (s, 1H), 3.71 - 3.52 (m, 8H), 3.49 (s, 3H), 3.26 - 3.21 (m, 1H), 3.04 - 2.97 (m, 2H), 2.92 (s, 3H), 2.71 - 2.58 (m, 2H), 2.37 - 2.26 (m, 1H), 2.22 - 2.11 (m, 2H), 2.11 - 2.00 (m, 1H), 1.90 - 1.76 (m, 2H), 0.81 - 0.64 (m, 3H). MS (ESI) m/z: 803 [M+H]⁺. |
| I-132 | 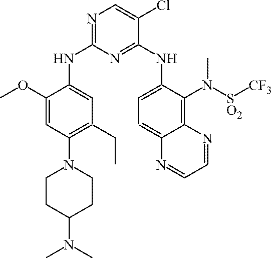 [SMILES: CCc1cc(Nc2ncc(Cl)c(Nc3ccc4nccnc4c3N(C)S(=O)(=O)C(F)(F)F)n2)c(OC)cc1N1CCC(N(C)C)CC1] | ¹H NMR (600 MHz, Methanol-d₄) δ 9.00 (d, J = 1.8 Hz, 1H), 8.98 (d, J = 1.8 Hz, 1H), 8.19 (s, 1H), 8.15 (d, J = 9.1 Hz, 1H), 8.08 (d, J = 9.2 Hz, 1H), 7.17 (s, 1H), 6.69 (s, 1H), 3.77 (s, 3H), 3.53 (s, 3H), 3.27 - 3.22 (m, 1H), 3.09 - 2.97 (m, 2H), 2.89 (s, 6H), 2.80 - 2.65 (m, 2H), 2.33 - 2.18 (m, 1H), 2.17 - 2.09 (m, 2H), 2.05 - 1.75 (m, 3H), 0.67 (d, J = 64.2 Hz, 3H). MS (ESI) m/z: 694 [M+H]⁺. |
| I-133 | 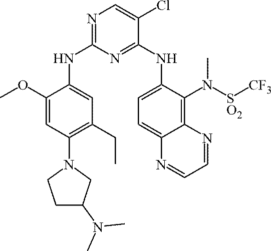 [SMILES: CCc1cc(Nc2ncc(Cl)c(Nc3ccc4nccnc4c3N(C)S(=O)(=O)C(F)(F)F)n2)c(OC)cc1N1CCC(N(C)C)C1] | ¹H NMR (600 MHz, MeOD) δ 9.01 (d, J = 1.7 Hz, 1H), 8.97 (d, J = 2.0 Hz, 1H), 8.19 (s, 1H), 8.17 - 8.10 (m, 2H), 7.19 (d, J = 8.6 Hz, 1H), 6.73 - 6.65 (m, 1H), 4.01 - 3.92 (m, 1H), 3.79 (s, 3H), 3.57 (s, 3H), 3.38 - 3.30 (m, 3H), 3.26 - 3.18 (m, 1H), 3.08 - 3.01 (m, 1H), 2.94 (s, 3H), 2.93 - 2.91 (m, 2H), 2.52 - 2.39 (m, 1H), 2.33 - 2.21 (m, 1H), 2.19 - 2.12 (m, 1H), 2.12 - 1.88 (m, 1H), 0.81 - 0.42 (m, 3H). MS (ESI) m/z: 680 [M+H]⁺. |
| I-134 | 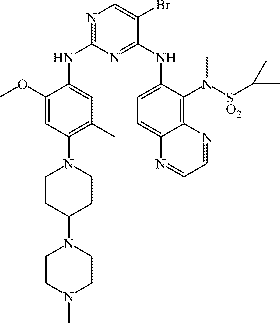 [SMILES: COc1cc(N2CCC(N3CCN(C)CC3)CC2)c(C)cc1Nc1ncc(Br)c(Nc2ccc3nccnc3c2N(C)S(=O)(=O)C(C)C)n1] | ¹H NMR (600 MHz, MeOD) δ 8.95 (d, J = 1.8 Hz, 1H), 8.89 (d, J = 1.9 Hz, 1H), 8.43 - 8.33 (m, 1H), 8.22 (s, 1H), 8.01 (d, J = 9.4 Hz, 1H), 7.23 (s, 1H), 6.73 (s, 1H), 3.77 (s, 3H), 3.71 - 3.48 (m, 8H), 3.40 (s, 3H), 3.40 - 3.37 (m, 1H), 3.35 - 3.28 (m, 1H), 3.25 - 3.18 (m, 2H), 2.93 (s, 3H), 2.87 - 2.77 (m, 2H), 2.22 - 2.12 (m, 2H), 2.04 - 1.81 (m, 5H), 1.59 (d, J = 6.6 Hz, 3H), 1.21 (d, J = 6.8 Hz, 3H). MS (ESI) m/z: 753 [M+H]⁺. |
| I-135 | 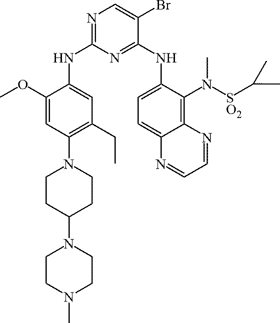 [SMILES: CCc1cc(Nc2ncc(Br)c(Nc3ccc4nccnc4c3N(C)S(=O)(=O)C(C)C)n2)c(OC)cc1N1CCC(N2CCN(C)CC2)CC1] | ¹H NMR (600 MHz, MeOD) δ 9.00 - 8.98 (m, 1H), 8.92 (d, J = 1.7 Hz, 1H), 8.43 - 8.35 (m, 1H), 8.26 (s, 1H), 8.04 (d, J = 9.3 Hz, 1H), 7.40 (s, 1H), 6.82 (s, 1H), 3.83 (s, 3H), 3.58 - 3.41 (m, 11H), 3.26 - 3.14 (m, 3H), 2.91 (s, 3H), 2.91 - 2.86 (m, 2H), 2.45 - 2.28 (m, 2H), 2.23 - 2.10 (m, 2H), 1.99 - 1.85 (m, 2H), 1.64 (d, J = 6.7 Hz, 3H), 1.26 (d, J = 6.9 Hz, 3H), 0.94 - 0.61 (m, 3H). MS (ESI) m/z: 767 [M+H]⁺. |
| I-136 | 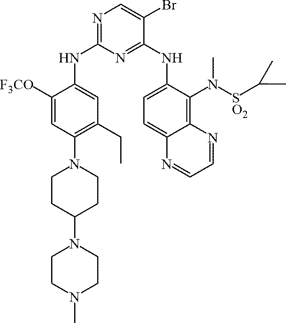 [SMILES: CCc1cc(Nc2ncc(Br)c(Nc3ccc4nccnc4c3N(C)S(=O)(=O)C(C)C)n2)c(OC(F)(F)F)cc1N1CCC(N2CCN(C)CC2)CC1] | MS (ESI) m/z: 821 [M+H]⁺. |
| I-137 | 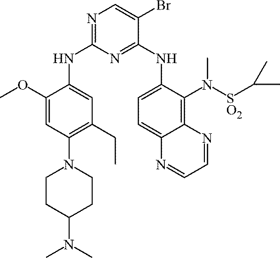 [SMILES: CCc1cc(Nc2ncc(Br)c(Nc3ccc4nccnc4c3N(C)S(=O)(=O)C(C)C)n2)c(OC)cc1N1CCC(N(C)C)CC1] | ¹H NMR (600 MHz, MeOD) δ 8.99 (d, J = 1.9 Hz, 1H), 8.93 (d, J = 1.8 Hz, 1H), 8.44 - 8.32 (m, 1H), 8.26 (s, 1H), 8.10 - 7.98 (m, 1H), 7.29 (s, 1H), 6.78 (s, 1H), 3.81 (s, 3H), 3.44 (s, 3H), 3.44 - 3.40 (m, 1H), 3.33 - 3.29 (m, 1H), 3.18 - 3.08 (m, 2H), 2.91 (s, 6H), 2.83 - 2.76 (m, 2H), 2.41 - 2.26 (m, 2H), 2.21 - 2.11 (m, 2H), 1.88 (s, 2H), 1.63 (d, J = 6.7 Hz, 3H), 1.25 (d, J = 6.9 Hz, 3H), 0.76 (t, J = 7.3 Hz, 3H). MS (ESI) m/z: 712 [M+H]⁺. |
| I-138 | 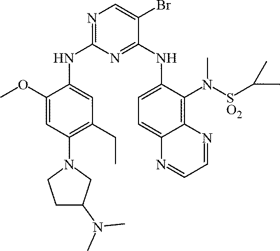 [SMILES: CCc1cc(Nc2ncc(Br)c(Nc3ccc4nccnc4c3N(C)S(=O)(=O)C(C)C)n2)c(OC)cc1N1CCC(N(C)C)C1] | ¹H NMR (600 MHz, Methanol-d₄) δ 8.99 (d, J = 1.9 Hz, 1H), 8.92 (d, J = 1.9 Hz, 1H), 8.50 - 8.35 (m, 1H), 8.26 (s, 1H), 8.02 (d, J = 8.8 Hz, 1H), 7.36 (d, J = 2.9 Hz, 1H), 6.77 (d, J = 2.5 Hz, 1H), 4.03 - 3.96 (m, 1H), 3.83 (s, 3H), 3.46 (s, 3H), 3.45 - 3.42 (m, 1H), 3.41 - 3.37 (m, 2H), 3.34 - 3.31 (m, 1H), 3.13 - 3.05 (m, 1H), 3.01 - 2.92 (m, 6H), 2.52 - 2.44 (m, 1H), 2.42 - 2.27 (m, 2H), 2.23 - 2.13 (m, 1H), 1.65 (d, J = 6.7 Hz, 3H), 1.26 (d, J = 6.9 Hz, 3H), 0.78 (br s, 3H). MS (ESI) m/z: 698 [M+H]⁺. |
| I-139 | 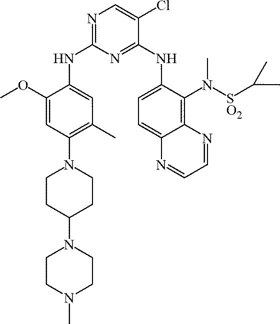 [SMILES: COc1cc(N2CCC(N3CCN(C)CC3)CC2)c(C)cc1Nc1ncc(Cl)c(Nc2ccc3nccnc3c2N(C)S(=O)(=O)C(C)C)n1] | MS (ESI) m/z: 709 [M+H]⁺. |
| I-140 | 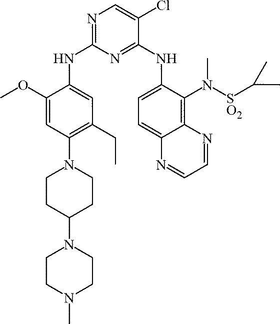 [SMILES: CCc1cc(Nc2ncc(Cl)c(Nc3ccc4nccnc4c3N(C)S(=O)(=O)C(C)C)n2)c(OC)cc1N1CCC(N2CCN(C)CC2)CC1] | MS (ESI) m/z: 723 [M+H]⁺. |
| I-141 | 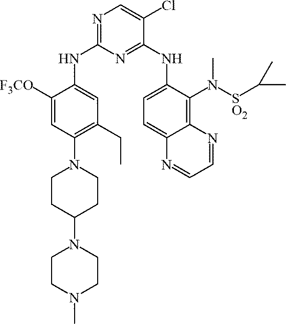 [SMILES: CCc1cc(Nc2ncc(Cl)c(Nc3ccc4nccnc4c3N(C)S(=O)(=O)C(C)C)n2)c(OC(F)(F)F)cc1N1CCC(N2CCN(C)CC2)CC1] | MS (ESI) m/z: 777 [M+H]⁺. |
| I-142 | 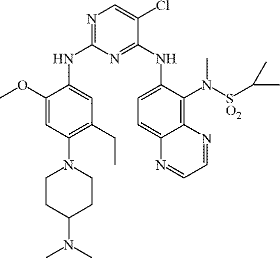 [SMILES: CCc1cc(Nc2ncc(Cl)c(Nc3ccc4nccnc4c3N(C)S(=O)(=O)C(C)C)n2)c(OC)cc1N1CCC(N(C)C)CC1] | ¹H NMR (600 MHz, MeOD) δ 8.99 (d, J = 1.9 Hz, 1H), 8.93 (d, J = 1.9 Hz, 1H), 8.40 - 8.30 (m, 1H), 8.19 (s, 1H), 8.04 (d, J = 9.4 Hz, 1H), 7.32 (s, 1H), 6.77 (s, 1H), 3.81 (s, 3H), 3.44 (s, 3H), 3.43 - 3.40 (m, 1H), 3.33 - 3.30 (m, 1H), 3.19 - 3.06 (m, 2H), 2.91 (s, 6H), 2.84 - 2.75 (m, 2H), 2.42 - 2.26 (m, 2H), 2.21 - 2.12 (m, 2H), 1.94 - 1.82 (m, 2H), 1.62 (d, J = 6.7 Hz, 3H), 1.25 (d, J = 6.9 Hz, 3H), 0.91 - 0.63 (m, 3H). MS (ESI) m/z: 668 [M+H]⁺. |
| I-143 | 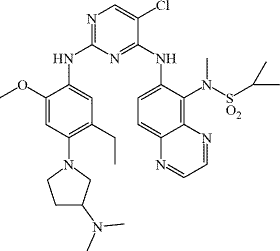 [SMILES: CCc1cc(Nc2ncc(Cl)c(Nc3ccc4nccnc4c3N(C)S(=O)(=O)C(C)C)n2)c(OC)cc1N1CCC(N(C)C)C1] | ¹H NMR (600 MHz, MeOD) δ 9.00 (d, J = 1.9 Hz, 1H), 8.93 (d, J = 1.8 Hz, 1H), 8.46 - 8.31 (m, 1H), 8.19 (s, 1H), 8.03 (d, J = 9.3 Hz, 1H), 7.33 (s, 1H), 6.79 - 6.74 (m, 1H), 4.02 - 3.95 (m, 1H), 3.83 (s, 3H), 3.45 (s, 3H), 3.45 - 3.41 (m, 1H), 3.41 - 3.37 (m, 2H), 3.29 - 3.25 (m, 1H), 3.14 - 3.04 (m, 1H), 2.96 (s, 6H), 2.51 - 2.43 (m, 1H), 2.42 - 2.29 (m, 2H), 2.23 - 2.11 (m, 1H), 1.63 (d, J = 6.7 Hz, 3H), 1.26 (d, J = 7.0 Hz, 3H), 0.90 - 0.67 (m, 3H). MS (ESI) m/z: 654 [M+H]⁺. |
| I-144 | 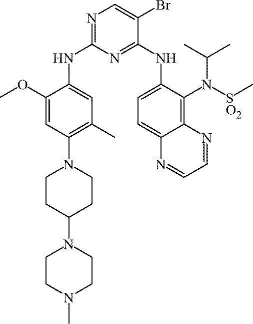 | MS (ESI) m/z: 753 [M+H]⁺. |
| I-145 | 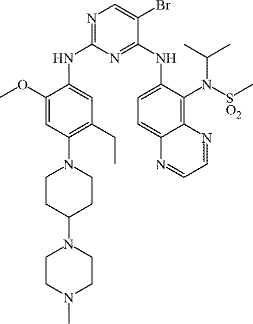 | MS (ESI) m/z: 767 [M+H]⁺. |
| I-146 | 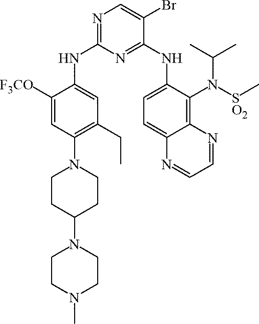 [SMILES: CCc1cc(Nc2ncc(Br)c(Nc3ccc4nccnc4c3N(C(C)C)S(C)(=O)=O)n2)c(OC(F)(F)F)cc1N1CCC(N2CCN(C)CC2)CC1] | MS (ESI) m/z: 821 [M+H]⁺. |
| I-147 | 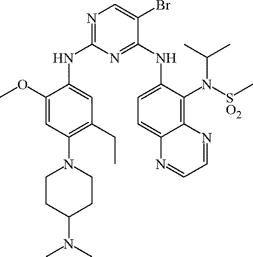 | MS (ESI) m/z: 712 [M+H]⁺. |
| I-148 | 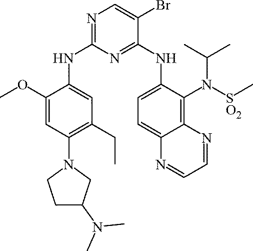 | MS (ESI) m/z: 698 [M+H]⁺. |
| I-149 | 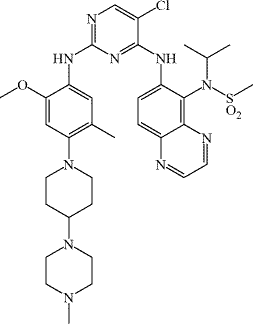 | MS (ESI) m/z: 709 [M+H]⁺. |
| I-150 | 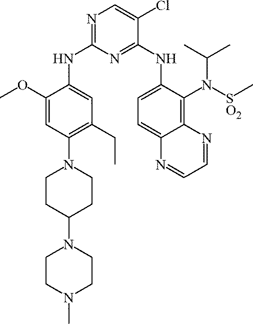 | MS (ESI) m/z: 723 [M+H]⁺. |
| I-151 | 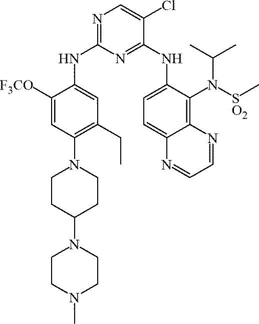 [SMILES: CCc1cc(Nc2ncc(Cl)c(Nc3ccc4nccnc4c3N(C(C)C)S(C)(=O)=O)n2)c(OC(F)(F)F)cc1N1CCC(N2CCN(C)CC2)CC1] | MS (ESI) m/z: 777 [M+H]⁺. |
| I-152 | 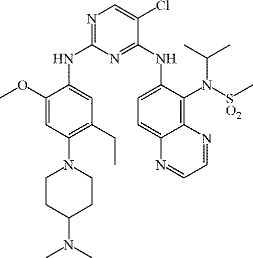 | MS (ESI) m/z: 668 [M+H]⁺. |
| I-153 | 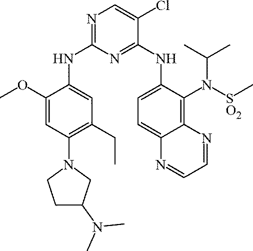 | MS (ESI) m/z: 654 [M+H]⁺. |
| I-154 | 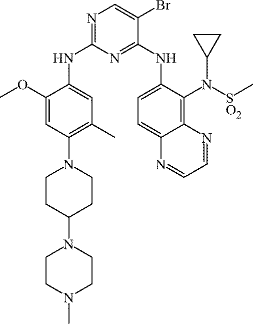 | ¹H NMR (600 MHz, Methanol-d₄) δ 8.96 (d, J = 1.9 Hz, 1H), 8.86 (d, J = 1.9 Hz, 1H), 8.77 - 8.65 (m, 1H), 8.22 (s, 1H), 7.96 (d, J = 9.4 Hz, 1H), 7.51 (s, 1H), 6.83 (s, 1H), 3.84 (s, 3H), 3.74 - 3.67 (m, 1H), 3.62 - 3.39 (m, 8H), 3.37 - 3.34 (m, 5H), 3.26 - 3.16 (m, 1H), 2.99 - 2.91 (m, 5H), 2.28 - 2.17 (m, 2H), 2.10 (s, 3H), 2.04 - 1.90 (m, 2H), 1.31 - 1.16 (m, 1H), 1.08 - 0.91 (m, 1H), 0.49 - 0.36 (m, 1H), 0.34 - 0.21 (m, 1H). MS (ESI) m/z: 751 [M+H]⁺. |
| I-155 | 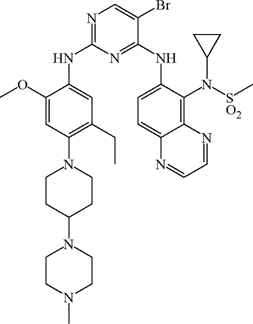 | ¹H NMR (600 MHz, Methanol-d₄) δ 8.97 (d, J = 1.9 Hz, 1H), 8.88 (d, J = 1.8 Hz, 1H), 8.72 - 8.56 (m, 1H), 8.24 (s, 1H), 7.98 (d, J = 9.3 Hz, 1H), 7.57 (s, 1H), 6.85 (s, 1H), 3.85 (s, 3H), 3.73 - 3.65 (m, 1H), 3.51 - 3.33 (m, 8H), 3.33 (s, 3H), 3.27 - 3.19 (m, 2H), 3.15 - 3.09 (m, 1H), 3.00 - 2.90 (m, 2H), 2.89 (s, 3H), 2.59 - 2.47 (m, 1H), 2.47 - 2.37 (m, 1H), 2.24 - 2.11 (m, 2H), 1.97 - 1.84 (m, 2H), 1.27 - 1.18 (m, 1H), 1.04 - 0.94 (m, 1H), 0.90 - 0.75 (m, 3H), 0.46 - 0.35 (m, 1H), 0.32 - 0.22 (m, 1H). MS (ESI) m/z: 765 [M+H]⁺. |
| I-156 | 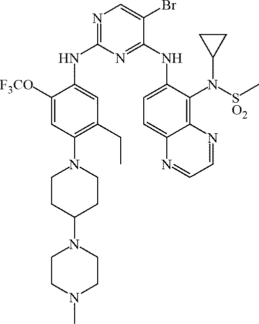 [SMILES: CCc1cc(Nc2ncc(Br)c(Nc3ccc4nccnc4c3N(C3CC3)S(C)(=O)=O)n2)c(OC(F)(F)F)cc1N1CCC(N2CCN(C)CC2)CC1] | ¹H NMR (600 MHz, Methanol-d₄) δ 8.94 (d, J = 1.9 Hz, 1H), 8.84 (d, J = 1.9 Hz, 1H), 8.67 (d, J = 9.4 Hz, 1H), 8.24 (s, 1H), 7.86 (d, J = 9.4 Hz, 1H), 7.57 (s, 1H), 7.06 (s, 1H), 3.75 - 3.64 (m, 1H), 3.39 - 3.23 (m, 11H), 3.20 - 3.14 (m, 2H), 3.07 - 2.93 (m, 1H), 2.84 (s, 3H), 2.80 - 2.73 (m, 2H), 2.63 - 2.50 (m, 2H), 2.16 - 2.09 (m, 2H), 1.92 - 1.79 (m, 2H), 1.29 - 1.22 (m, 1H), 1.02 - 0.88 (m, 4H), 0.44 - 0.33 (m, 1H), 0.33 - 0.19 (m, 1H). MS (ESI) m/z: 819 [M+H]⁺. |
| I-157 | 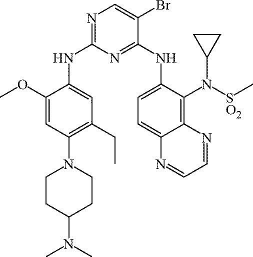 | ¹H NMR (600 MHz, DMSO-d₆) δ 10.01 (s, 1H), 8.99 (d, J = 1.9 Hz, 1H), 8.93 (d, J = 1.9 Hz, 1H), 8.90 - 8.74 (m, 1H), 8.48 (s, 1H), 8.31 (d, J = 2.2 Hz, 2H), 7.90 (d, J = 8.7 Hz, 1H), 7.40 (s, 1H), 6.82 (s, 1H), 3.78 (s, 3H), 3.65 - 3.54 (m, 1H), 3.38 (s, 3H), 3.33 - 3.23 (m, 1H), 3.12 (d, J = 10.7 Hz, 2H), 2.84 (s, 6H), 2.78 (t, J = 11.9 Hz, 2H), 2.55 (t, J = 7.3 Hz, 1H), 2.47 (t, J = 7.2 Hz, 1H), 2.16 - 2.00 (m, 2H), 1.88 - 1.77 (m, 2H), 1.28 - 1.17 (m, 1H), 1.01 - 0.96 (m, 1H), 0.95 - 0.84 (m, 3H), 0.47 - 0.37 (m, 1H), 0.31 - 0.21 (m, 1H). MS (ESI) m/z: 710 [M+H]⁺. |
| I-158 | 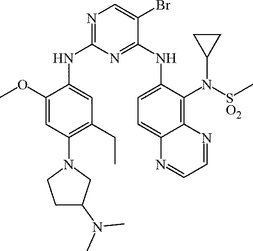 | ¹H NMR (600 MHz, Methanol-d₄) δ 8.94 (d, J = 1.9 Hz, 1H), 8.84 (d, J = 1.9 Hz, 1H), 8.76 - 8.62 (m, 1H), 8.20 (s, 1H), 7.94 (dd, J = 9.2, 1.9 Hz, 1H), 7.62 (d, J = 4.7 Hz, 1H), 6.80 (s, 1H), 4.04 - 3.97 (m, 1H), 3.84 (s, 3H), 3.71 - 3.64 (m, 1H), 3.44 - 3.36 (m, 2H), 3.33 (s, 3H), 3.30 - 3.26 (m, 1H), 3.15 - 3.04 (m, 1H), 3.00 - 2.94 (m, 6H), 2.56 - 2.37 (m, 3H), 2.25 - 2.12 (m, 1H), 1.30 - 1.23 (m, 1H), 1.01 - 0.94 (m, 1H), 0.87 - 0.74 (m, 3H), 0.43 - 0.34 (m, 1H), 0.34 - 0.25 (m, 1H). MS (ESI) m/z: 696 [M+H]⁺. |
| I-159 | 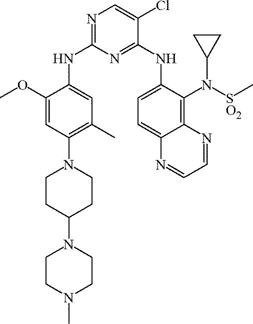 | ¹H NMR (600 MHz, Methanol-d₄) δ 8.98 (d, J = 1.9 Hz, 1H), 8.89 (d, J = 1.9 Hz, 1H), 8.70 (d, J = 10.5 Hz, 1H), 8.17 (s, 1H), 8.01 (d, J = 9.4 Hz, 1H), 7.54 (s, 1H), 6.86 (s, 1H), 3.87 (s, 3H), 3.77 - 3.68 (m, 1H), 3.65 - 3.42 (m, 8H), 3.41 - 3.35 (m, 5H), 3.31 - 3.21 (m, 1H), 3.05 - 2.91 (m, 5H), 2.29 - 2.17 (m, 2H), 2.12 (s, 3H), 2.07 - 1.92 (m, 2H), 1.28 - 1.12 (m, 1H), 1.05 - 0.95 (m, 1H), 0.53 - 0.39 (m, 1H), 0.38 - 0.25 (m, 1H). MS (ESI) m/z: 707 [M+H]⁺. |
| I-160 | 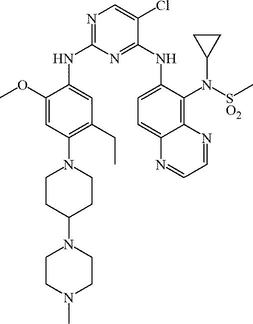 | ¹H NMR (600 MHz, Methanol-d₄) δ 8.98 (d, J = 1.8 Hz, 1H), 8.90 (d, J = 1.9 Hz, 1H), 8.60 (s, 1H), 8.19 (s, 1H), 8.01 (d, J = 9.3 Hz, 1H), 7.53 (s, 1H), 6.87 (s, 1H), 3.85 (s, 3H), 3.72 - 3.66 (m, 1H), 3.65 - 3.44 (m, 8H), 3.35 (s, 3H), 3.30 - 3.21 (m, 3H), 3.03 - 2.90 (m, 5H), 2.58 - 2.48 (m, 1H), 2.49 - 2.40 (m, 1H), 2.29 - 2.15 (m, 2H), 2.06 - 1.89 (m, 2H), 1.23 - 1.07 (m, 1H), 1.04 - 0.94 (m, 1H), 0.94 - 0.75 (m, 3H), 0.46 - 0.35 (m, 1H), 0.32 - 0.23 (m, 1H). MS (ESI) m/z: 721 [M+H]⁺. |
| I-161 | 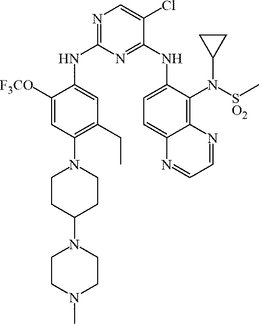 [SMILES: CCc1cc(Nc2ncc(Cl)c(Nc3ccc4nccnc4c3N(C3CC3)S(C)(=O)=O)n2)c(OC(F)(F)F)cc1N1CCC(N2CCN(C)CC2)CC1] | ¹H NMR (600 MHz, Methanol-d₄) δ 8.95 (d, J = 1.9 Hz, 1H), 8.86 (d, J = 1.9 Hz, 1H), 8.62 (d, J = 9.1 Hz, 1H), 8.18 (s, 1H), 7.89 (d, J = 9.3 Hz, 1H), 7.57 (s, 1H), 7.15 - 7.02 (m, 1H), 3.71 - 3.64 (m, 1H), 3.63 - 3.43 (m, 8H), 3.35 (s, 3H), 3.27 - 3.15 (m, 3H), 2.93 (s, 3H), 2.86 - 2.76 (m, 2H), 2.66 - 2.49 (m, 2H), 2.28 - 2.15 (m, 2H), 2.02 - 1.87 (m, 2H), 1.26 - 1.11 (m, 1H), 1.04 - 0.90 (m, 4H), 0.46 - 0.34 (m, 1H), 0.30 - 0.21 (m, 1H). MS (ESI) m/z: 775 [M+H]⁺. |
| I-162 | 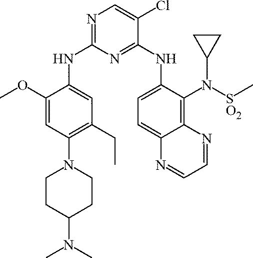 | ¹H NMR (600 MHz, Methanol-d₄) δ 8.99 (d, J = 1.8 Hz, 1H), 8.92 (d, J = 1.8 Hz, 1H), 8.67 - 8.49 (m, 1H), 8.21 (s, 1H), 8.01 (d, J = 9.4 Hz, 1H), 7.43 (s, 1H), 6.84 (s, 1H), 3.84 (s, 3H), 3.73 - 3.67 (m, 1H), 3.35 (s, 3H), 3.31 - 3.30 (m, 1H), 3.25 - 3.15 (m, 2H), 2.94 (s, 6H), 2.86 (t, J = 11.6 Hz, 2H), 2.57 - 2.41 (m, 2H), 2.27 - 2.12 (m, 2H), 2.00 - 1.83 (m, 2H), 1.19 - 1.10 (m, 1H), 1.04 - 0.94 (m, 1H), 0.95 - 0.72 (m, 3H), 0.47 - 0.35 (m, 1H), 0.32 - 0.22 (m, 1H). MS (ESI) m/z: 666 [M+H]⁺. |
| I-163 | 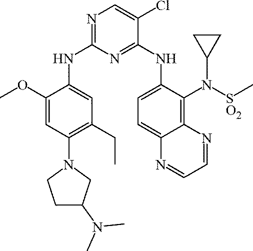 | ¹H NMR (600 MHz, Methanol-d₄) δ 8.98 (d, J = 1.7 Hz, 1H), 8.94 - 8.89 (m, 1H), 8.69 - 8.51 (m, 1H), 8.18 (s, 1H), 7.98 (d, J = 9.3 Hz, 1H), 7.44 (d, J = 3.9 Hz, 1H), 6.81 (s, 1H), 4.10 - 3.97 (m, 1H), 3.85 (s, 3H), 3.72 - 3.63 (m, 1H), 3.49 - 3.40 (m, 2H), 3.37 - 3.35 (m, 1H), 3.32 (s, 3H), 3.20 - 3.08 (m, 1H), 2.98 (s, 6H), 2.59 - 2.39 (m, 3H), 2.30 - 2.17 (m, 1H), 1.21 - 1.12 (m, 1H), 1.02 - 0.94 (m, 1H), 0.93 - 0.77 (m, 3H), 0.47 - 0.34 (m, 1H), 0.34 - 0.22 (m, 1H). MS (ESI) m/z: 652 [M+H]⁺. |
| I-164 | 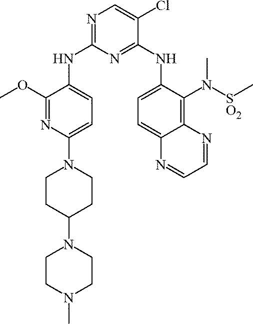 | ¹H NMR (600 MHz, Methanol-d₄) δ 8.93 (d, J = 1.7 Hz, 1H), 8.88 (d, J = 1.8 Hz, 1H), 8.64 - 8.35 (m, 1H), 8.21 - 8.07 (m, 1H), 8.07 - 7.95 (m, 1H), 7.47 - 7.40 (m, 1H), 6.37 - 6.18 (m, 1H), 4.58 - 4.40 (m, 2H), 3.82 (s, 3H), 3.68 - 3.56 (m, 8H), 3.50 - 3.46 (m, 1H), 3.43 (s, 3H), 3.12 (s, 3H), 2.93 (s, 3H), 2.91 - 2.84 (m, 2H), 2.27 - 2.07 (m, 2H), 1.84 - 1.62 (m, 2H). MS (ESI) m/z: 668 [M+H]⁺. |
| I-165 | 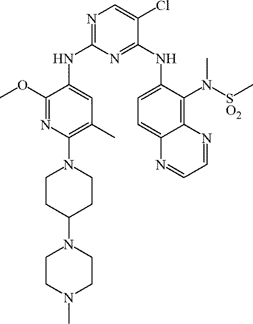 | ¹H NMR (600 MHz, Methanol-d₄) δ 9.00 (d, J = 1.7 Hz, 1H), 8.94 (d, J = 1.8 Hz, 1H), 8.68 - 8.52 (m, 1H), 8.22 (d, J = 6.0 Hz, 1H), 8.17 - 7.96 (m, 1H), 7.46 (s, 1H), 3.96 (s, 3H), 3.88 - 3.73 (m, 2H), 3.69 - 3.53 (m, 8H), 3.52 (s, 3H), 3.33 - 3.28 (m, 1H), 3.21 (s, 3H), 2.95 (s, 3H), 2.94 - 2.87 (m, 2H), 2.23 - 2.07 (m, 2H), 2.02 (s, 3H), 1.86 - 1.62 (m, 2H). MS (ESI) m/z: 682 [M+H]⁺. |
| I-166 | 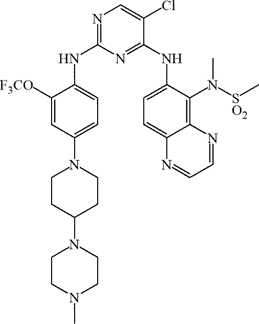 [SMILES: CN1CCN(C2CCN(c3ccc(Nc4ncc(Cl)c(Nc5ccc6nccnc6c5N(C)S(C)(=O)=O)n4)c(OC(F)(F)F)c3)CC2)CC1] | ¹H NMR (600 MHz, Methanol-d₄) δ 8.97 (d, J = 1.9 Hz, 1H), 8.91 (d, J = 1.9 Hz, 1H), 8.54 (d, J = 9.4 Hz, 1H), 8.21 (s, 1H), 7.99 (d, J = 9.4 Hz, 1H), 7.45 (d, J = 8.6 Hz, 1H), 7.03 - 6.95 (m, 2H), 3.95 - 3.88 (m, 2H), 3.68 - 3.53 (m, 8H), 3.45 (s, 3H), 3.38 - 3.34 (m, 1H), 3.17 (s, 3H), 2.97 (s, 3H), 2.92 (t, J = 12.4 Hz, 2H), 2.31 - 2.18 (m, 2H), 2.01 - 1.80 (m, 2H). MS (ESI) m/z: 721 [M+H]⁺. |
| I-167 | 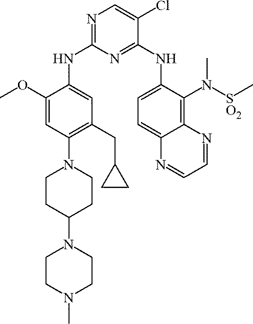 | ¹H NMR (600 MHz, Methanol-d₄) δ 8.98 (d, J = 1.8 Hz, 1H), 8.92 (d, J = 1.9 Hz, 1H), 8.61 - 8.44 (m, 1H), 8.19 (s, 1H), 8.03 (d, J = 9.3 Hz, 1H), 7.60 (s, 1H), 6.84 (s, 1H), 3.84 (s, 3H), 3.45 (s, 3H), 3.44 - 3.32 (m, 8H), 3.18 (s, 3H), 3.16 - 3.13 (m, 3H), 2.94 - 2.86 (m, 5H), 2.38 - 2.23 (m, 2H), 2.18 - 2.08 (m, 2H), 1.97 - 1.76 (m, 2H), 0.75 - 0.52 (m, 1H), 0.36 - 0.11 (m, 2H), - 0.08 - -0.30 (m, 2H). MS (ESI) m/z: 721 [M+H]⁺. |
| I-168 | 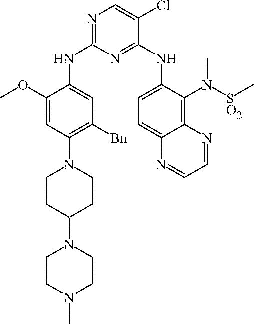 | ¹H NMR (600 MHz, Methanol-d₄) δ 8.90 (d, J = 1.8 Hz, 1H), 8.82 (d, J = 1.8 Hz, 1H), 8.48 - 8.37 (m, 1H), 8.18 (s, 1H), 7.99 (d, J = 9.4 Hz, 1H), 7.46 (s, 1H), 7.07 - 6.98 (m, 3H), 6.90 - 6.79 (m, 3H), 3.84 (s, 3H), 3.77 - 3.68 (m, 2H), 3.66 - 3.56 (m, 8H), 3.41 (s, 3H), 3.29 - 3.24 (m, 1H), 3.12 (s, 3H), 3.02 - 2.92 (m, 5H), 2.86 - 2.67 (m, 2H), 2.18 - 2.03 (m, 2H), 1.89 - 1.70 (m, 2H). MS (ESI) m/z: 757 [M+H]⁺. |
| I-169 | 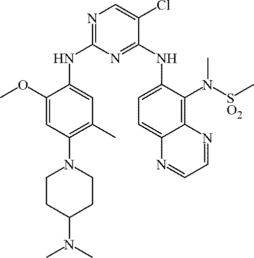 | ¹H NMR (600 MHz, Methanol-d₄) δ 8.96 (d, J = 1.8 Hz, 1H), 8.89 (d, J = 1.8 Hz, 1H), 8.67 - 8.51 (m, 1H), 8.15 (s, 1H), 8.02 (d, J = 9.3 Hz, 1H), 7.33 (s, 1H), 6.74 (s, 1H), 3.81 (s, 3H), 3.44 (s, 3H), 3.34 - 3.30 (m, 1H), 3.26 - 3.18 (m, 2H), 3.15 (s, 3H), 2.92 (s, 6H), 2.83 - 2.61 (m, 2H), 2.24 - 2.15 (m, 2H), 2.00 (br s, 3H), 1.94 - 1.85 (m, 2H). MS (ESI) m/z: 626 [M+H]⁺. |
| I-170 | 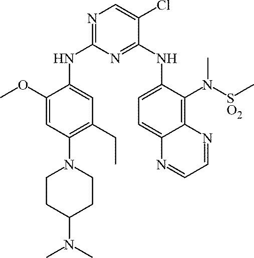 | ¹H NMR (600 MHz, Methanol-d₄) δ 8.94 (d, J = 1.8 Hz, 1H), 8.88 (d, J = 1.8 Hz, 1H), 8.51 - 8.38 (m, 1H), 8.15 (s, 1H), 7.98 (d, J = 9.3 Hz, 1H), 7.32 (s, 1H), 6.76 (s, 1H), 3.79 (s, 3H), 3.41 (s, 3H), 3.33 - 3.28 (m, 1H), 3.17 - 3.08 (m, 5H), 2.89 (s, 6H), 2.85 - 2.71 (m, 2H), 2.48 - 2.25 (m, 2H), 2.18 - 2.08 (m, 2H), 1.92 - 1.78 (m, 2H), 0.92 - 0.70 (m, 3H). MS (ESI) m/z: 640 [M+H]⁺. |
| I-171 | 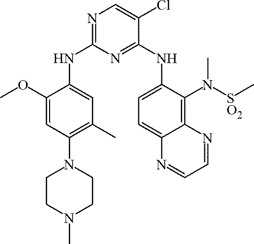 | ¹H NMR (600 MHz, Methanol-d₄) δ 8.93 (d, J = 1.8 Hz, 1H), 8.86 (d, J = 1.9 Hz, 1H), 8.62 - 8.42 (m, 1H), 8.13 (s, 1H), 7.98 (d, J = 9.6 Hz, 1H), 7.35 (s, 1H), 6.73 (s, 1H), 3.80 (s, 3H), 3.62 - 3.53 (m, 2H), 3.41 (s, 3H), 3.31 - 3.27 (m, 2H), 3.24 - 3.17 (m, 2H), 3.13 (s, 3H), 3.11 - 3.04 (m, 2H), 2.96 (s, 3H), 1.97 (s, 3H). MS (ESI) m/z: 598 [M+H]⁺. |
| I-172 | 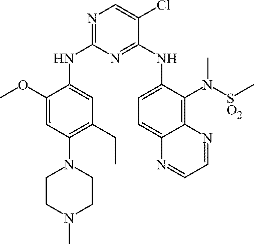 | ¹H NMR (600 MHz, Methanol-d₄) δ 8.97 (d, J = 1.8 Hz, 1H), 8.90 (d, J = 1.7 Hz, 1H), 8.66 - 8.52 (m, 1H), 8.18 (s, 1H), 8.02 (d, J = 9.3 Hz, 1H), 7.53 (s, 1H), 6.82 (s, 1H), 3.85 (s, 3H), 3.65 - 3.54 (m, 2H), 3.46 (s, 3H), 3.36 - 3.31 (m, 2H), 3.21 - 3.17 (m, 2H), 3.16 (s, 3H), 3.15 - 3.08 (m, 2H), 2.98 (s, 3H), 2.52 - 2.32 (m, 2H), 0.96 - 0.71 (m, 3H). MS (ESI) m/z: 612 [M+H]⁺. |
| I-173 | 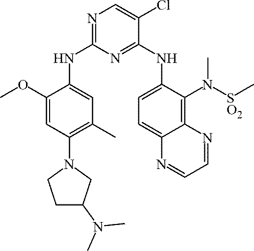 | ¹H NMR (600 MHz, Methanol-d₄) δ 8.95 (d, J = 1.9 Hz, 1H), 8.89 (d, J = 1.9 Hz, 1H), 8.55 - 8.40 (m, 1H), 8.15 (br s, 1H), 8.07 - 7.96 (m, 1H), 7.20 (s, 1H), 6.68 (d, J = 1.7 Hz, 1H), 4.03 - 3.96 (m, 1H), 3.80 (s, 3H), 3.43 (s, 3H), 3.41 - 3.38 (m, 1H), 3.37 - 3.32 (m, 2H), 3.33 - 3.31 (m, 1H), 3.13 (s, 3H), 2.95 (s, 6H), 2.53 - 2.43 (m, 1H), 2.24 - 2.17 (m, 1H), 2.00 (br s, 3H). MS (ESI) m/z: 612 [M+H]⁺. |
| I-174 | 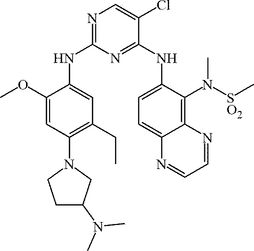 | ¹H NMR (600 MHz, Methanol-d₄) δ 8.98 (d, J = 1.7 Hz, 1H), 8.91 (d, J = 1.7 Hz, 1H), 8.61 - 8.43 (m, 1H), 8.18 (s, 1H), 8.01 (d, J = 9.3 Hz, 1H), 7.36 (s, 1H), 6.79 (s, 1H), 4.06 - 3.99 (m, 1H), 3.84 (s, 3H), 3.46 (s, 3H), 3.44 - 3.40 (m, 2H), 3.36 - 3.33 (m, 1H), 3.16 (s, 3H), 3.14 - 3.09 (m, 1H), 2.98 (s, 6H), 2.55 - 2.48 (m, 1H), 2.50 - 2.38 (m, 2H), 2.30 - 2.14 (m, 1H), 0.99 - 0.78 (m, 3H). MS (ESI) m/z: 626 [M+H]⁺. |
| I-175 | 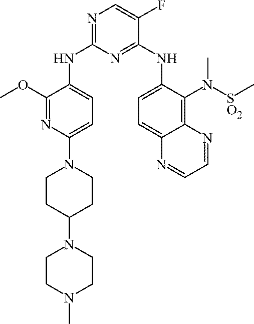 | ¹H NMR (600 MHz, Methanol-d₄) δ 8.88 (d, J = 1.7 Hz, 1H), 8.84 (d, J = 1.8 Hz, 1H), 8.39 - 8.18 (m, 1H), 8.11 - 7.89 (m, 2H), 7.31 (d, J = 8.5 Hz, 1H), 6.35 - 6.04 (m, 1H), 4.56 - 4.36 (m, 2H), 3.75 (s, 3H), 3.68 - 3.52 (m, 8H), 3.50 - 3.44 (m, 1H), 3.38 (s, 3H), 3.01 (s, 3H), 2.88 (s, 3H), 2.85 - 2.72 (m, 2H), 2.21 - 2.06 (m, 2H), 1.78 - 1.58 (m, 2H). MS (ESI) m/z: 652 [M+H]⁺. |
| I-176 | 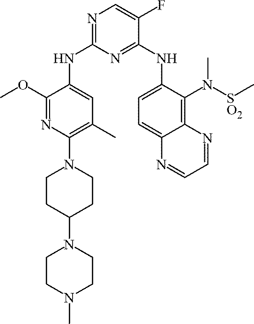 | ¹H NMR (600 MHz, Methanol-d₄) δ 8.95 (d, J = 1.8 Hz, 1H), 8.91 (d, J = 1.8 Hz, 1H), 8.32 (d, J = 9.3 Hz, 1H), 8.15 - 7.98 (m, 2H), 7.35 (s, 1H), 3.88 (s, 3H), 3.78 - 3.64 (m, 2H), 3.57 - 3.37 (m, 11H), 3.22 - 3.16 (m, 1H), 3.11 (s, 3H), 2.88 (s, 3H),2.86 - 2.73 (m, 2H), 2.08 - 1.99 (m, 2H), 2.00 - 1.84 (m, 3H), 1.75 - 1.52 (m, 2H). MS (ESI) m/z: 666 [M+H]⁺. |
| I-177 | 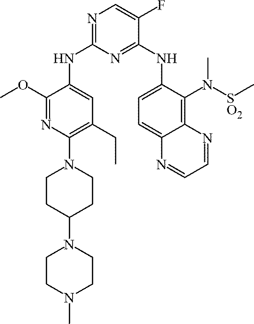 | ¹H NMR (600 MHz, Methanol-d₄) δ 8.96 (d, J = 1.8 Hz, 1H), 8.92 (d, J = 1.8 Hz, 1H), 8.24 - 8.16 (m, 1H), 8.10 (d, J = 4.8 Hz, 1H), 8.04 (d, J = 9.1 Hz, 1H), 7.58 (s, 1H), 3.87 (s, 3H), 3.73 - 3.53 (m, 8H), 3.47 - 3.39 (m, 5H), 3.35 - 3.29 (m, 1H), 3.08 (s, 3H), 2.93 (s, 3H), 2.92 - 2.83 (m, 2H), 2.34 - 2.22 (m, 1H), 2.18 - 2.09 (m, 2H), 2.09 - 2.01 (m, 1H), 1.96 - 1.75 (m, 2H), 0.77 - 0.55 (m, 3H). MS (ESI) m/z: 680 [M+H]⁺. |
| I-178 | 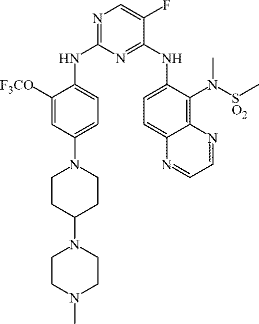 [SMILES: CN1CCN(C2CCN(c3ccc(Nc4ncc(F)c(Nc5ccc6nccnc6c5N(C)S(C)(=O)=O)n4)c(OC(F)(F)F)c3)CC2)CC1] | ¹H NMR (600 MHz, Methanol-d₄) δ 8.99 (d, J = 1.7 Hz, 1H), 8.95 (d, J = 1.7 Hz, 1H), 8.32 (d, J = 9.2 Hz, 1H), 8.13 (d, J = 4.7 Hz, 1H), 8.06 (d, J = 9.1 Hz, 1H), 7.37 (d, J = 9.0 Hz, 1H), 6.95 (s, 1H), 6.90 (d, J = 8.6 Hz, 1H), 3.97 - 3.85 (m, 2H), 3.69 - 3.53 (m, 8H), 3.45 (s, 3H), 3.13 (s, 3H), 2.97 (s, 3H), 2.88 (t, J = 13.1 Hz, 2H), 2.28 - 2.18 (m, 2H), 1.95 - 1.71 (m, 2H). MS (ESI) m/z: 705 [M+H]⁺. |
| I-179 | 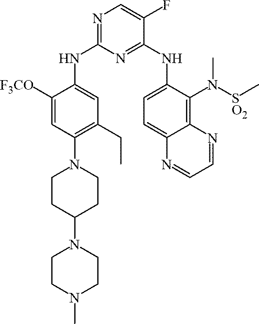 [SMILES: CCc1cc(Nc2ncc(F)c(Nc3ccc4nccnc4c3N(C)S(C)(=O)=O)n2)c(OC(F)(F)F)cc1N1CCC(N2CCN(C)CC2)CC1] | ¹H NMR (600 MHz, Methanol-d₄) δ 9.01 - 8.99 (m, 1H), 8.97 (d, J = 1.9 Hz, 1H), 8.19 (d, J = 4.9 Hz, 1H), 8.12 (d, J = 9.2 Hz, 1H), 8.01 (d, J = 9.2 Hz, 1H), 7.35 (s, 1H), 7.03 (s, 1H), 3.86 - 3.68 (m, 8H), 3.49 - 3.45 (m, 1H), 3.43 (s, 3H), 3.11 (s, 3H), 3.11 - 3.06 (m, 2H), 3.02 (s, 3H), 2.82 - 2.65 (m, 2H), 2.53 - 2.36 (m, 1H), 2.38 - 2.23 (m, 2H), 2.19 (s, 1H), 2.03 - 1.87 (m, 2H), 0.87 - 0.60 (m, 3H). MS (ESI) m/z: 733 [M+H]⁺. |
| I-180 | 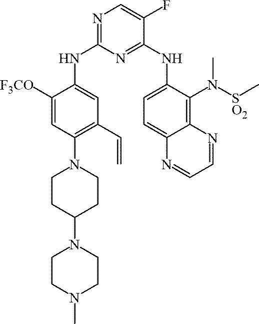 [SMILES: C=Cc1cc(Nc2ncc(F)c(Nc3ccc4nccnc4c3N(C)S(C)(=O)=O)n2)c(OC(F)(F)F)cc1N1CCC(N2CCN(C)CC2)CC1] | ¹H NMR (600 MHz, Methanol-d₄) δ 8.97 (d, J = 1.9 Hz, 1H), 8.91 (d, J = 1.8 Hz, 1H), 8.28 (d, J = 9.3 Hz, 1H), 8.16 (d, J = 4.3 Hz, 1H), 7.91 (d, J = 9.2 Hz, 1H), 7.70 (s, 1H), 6.98 (s, 1H), 6.72 (dd, J = 17.7, 11.0 Hz, 1H), 5.21 (d, J = 17.7 Hz, 1H), 4.85 (d, J = 11.0 Hz, 1H), 3.67 - 3.48 (m, 8H), 3.43 (s, 3H), 3.27 - 3.19 (m, 3H), 3.13 (s, 3H), 2.96 (s, 3H), 2.72 (td, J = 11.8, 2.2 Hz, 2H), 2.23 - 2.12 (m, 2H), 2.03 - 1.80 (m, 2H). MS (ESI) m/z: 731 [M+H]⁺. |
| I-181 | 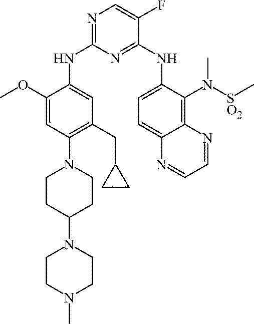 | ¹H NMR (600 MHz, Methanol-d₄) δ 8.95 - 8.89 (m, 1H), 8.90 - 8.84 (m, 1H), 8.24 - 8.11 (m, 1H), 8.08 - 8.01 (m, 1H), 7.99 (d, J = 9.1 Hz, 1H), 7.35 (s, 1H), 6.72 (s, 1H), 3.74 (s, 3H), 3.70 - 3.51 (m, 8H), 3.37 (s, 3H), 3.34 - 3.26 (m, 1H), 3.08 - 3.03 (m, 2H), 3.02 (s, 3H), 2.91 (s, 3H), 2.75 (dt, J = 25.4, 11.7 Hz, 2H), 2.27 - 2.04 (m, 3H), 1.97 - 1.73 (m, 3H), 0.55 - 0.33 (m, 1H), 0.24 - 0.00 (m, 2H), -0.13 - - 0.41 (m, 2H). MS (ESI) m/z: 705 [M+H]⁺. |
| I-182 | 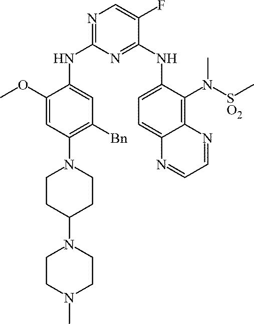 | ¹H NMR (600 MHz, Methanol-d₄) δ 8.90 (d, J = 1.9 Hz, 1H), 8.85 (d, J = 1.9 Hz, 1H), 8.29 (d, J = 9.2 Hz, 1H), 8.13 (d, J = 4.7 Hz, 1H), 8.06 (d, J = 9.2 Hz, 1H), 7.41 (s, 1H), 7.12 - 6.95 (m, 3H), 6.89 - 6.77 (m, 3H), 3.84 (s, 3H), 3.74 - 3.61 (m, 2H), 3.59 - 3.46 (m, 8H), 3.42 (s, 3H), 3.23 - 3.15 (m, 1H), 3.09 (s, 3H), 2.99 - 2.88 (m, 5H), 2.82 - 2.66 (m, 2H), 2.11 - 1.98 (m, 2H), 1.85 - 1.68 (m, 2H). MS (ESI) m/z: 741 [M+H]⁺. |
| I-183 | 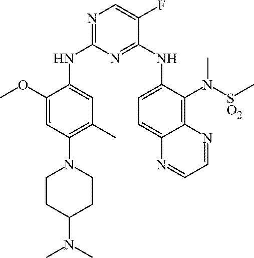 | ¹H NMR (600 MHz, DMSO-d₆) δ 9.23 (s, 1H), 9.01 (d, J = 1.8 Hz, 1H), 8.97 (d, J =1.9 Hz, 1H), 8.57 (d, J = 9.3 Hz, 1H), 8.47 (s, 1H), 8.27 (d, J = 3.7 Hz, 1H), 8.06 (d, J = 9.3 Hz, 1H), 7.39 (s, 1H), 6.70 (s, 1H), 3.78 (s, 3H), 3.35 (s, 3H), 3.31 - 3.25 (m, 1H), 3.18 (s, 3H), 3.15 - 3.08 (m, 2H), 2.82 (s, 6H), 2.71 - 2.61 (m, 2H), 2.18 - 2.03 (m, 2H), 1.91 (s, 3H), 1.83 - 1.71 (m, 2H). MS (ESI) m/z: 610 [M+H]⁺. |
| I-184 | 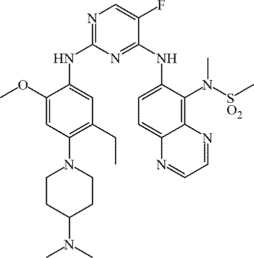 | 1H NMR (600 MHz, DMSO-d₆) δ 9.57 - 9.41 (m, 1H), 9.02 (d, J = 1.9 Hz, 1H), 8.99 (d, J = 1.9 Hz, 1H), 8.62 (s, 1H), 8.46 (d, J = 9.1 Hz, 1H), 8.31 (d, J = 3.9 Hz, 1H), 8.06 (d, J = 9.2 Hz, 1H), 7.41 (s, 1H), 6.74 (s, 1H), 3.78 (s, 3H), 3.33 (s, 3H), 3.30 - 3.23 (m, 1H), 3.16 (s, 3H), 3.09 - 2.97 (m, 2H), 2.82 (s, 6H), 2.77 - 2.65 (m, 2H), 2.41 - 2.29 (m, 1H), 2.27 - 2.15 (m, 1H), 2.13 - 2.05 (m, 2H), 1.86 - 1.63 (m, 2H), 0.88 - 0.61 (m, 3H). MS (ESI) m/z: 624 [M+H]⁺. |
| I-185 | 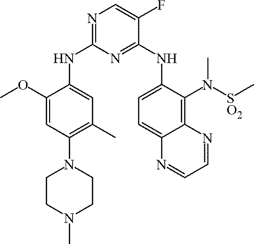 | ¹H NMR (600 MHz, Methanol-d₄) δ 8.99 (d, J = 1.8 Hz, 1H), 8.94 (d, J = 1.8 Hz, 1H), 8.41 (d, J = 9.9 Hz, 1H), 8.21 - 7.99 (m, 2H), 7.37 (s, 1H), 6.76 (s, 1H), 3.84 (s, 3H), 3.69 - 3.56 (m, 2H), 3.47 (s, 3H), 3.36 - 3.32 (m, 2H), 3.24 - 3.18 (m, 2H), 3.14 (s, 3H), 3.13 - 3.05 (m, 2H), 2.99 (s, 3H), 1.93 (s, 3H). MS (ESI) m/z: 582 [M+H]⁺. |
| I-186 | 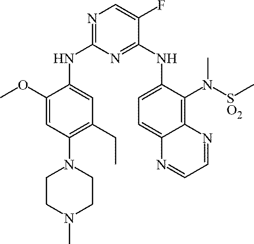 | ¹H NMR (600 MHz, Methanol-d₄) δ 9.01 (d, J = 1.9 Hz, 1H), 8.96 (d, J = 1.8 Hz, 1H), 8.27 (d, J = 9.1 Hz, 1H), 8.14 (d, J = 4.8 Hz, 1H), 8.09 (d, J = 9.1 Hz, 1H), 7.35 (s, 1H), 6.81 (s, 1H), 3.85 (s, 3H), 3.65 - 3.53 (m, 2H), 3.46 (s, 3H), 3.30 - 3.26 (m, 2H), 3.17 - 3.10 (m, 7H), 2.98 (s, 3H), 2.47 - 2.31 (m, 1H), 2.28 - 2.09 (m, 1H), 0.90 - 0.50 (m, 3H). MS (ESI) m/z: 596 [M+H]⁺. |
| I-187 | 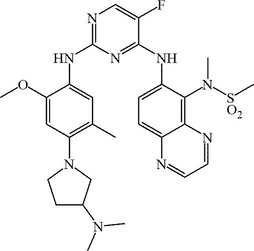 | ¹H NMR (600 MHz, Methanol-d₄) δ 9.00 (d, J = 1.8 Hz, 1H), 8.95 (d, J = 1.8 Hz, 1H), 8.37 (d, J = 9.5 Hz, 1H), 8.10 (d, J = 7.9 Hz, 2H), 7.21 (s, 1H), 6.69 (s, 1H), 4.05 - 3.96 (m, 1H), 3.83 (s, 3H), 3.48 (d, J = 2.9 Hz, 3H), 3.47 - 3.44 (m, 1H), 3.42 - 3.34 (m, 2H), 3.20 - 3.13 (m, 3H), 3.11 (t, J = 8.7 Hz, 1H), 3.03 - 2.94 (m, 6H), 2.57 - 2.44 (m, 1H), 2.30 - 2.16 (m, 1H), 1.96 (s, 3H). MS (ESI) m/z: 596 [M+H]⁺. |
| I-188 | 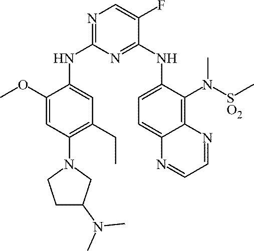 | ¹H NMR (600 MHz, Methanol-d₄) δ 9.00 (d, J = 1.7 Hz, 1H), 8.96 (d, J = 1.8 Hz, 1H), 8.34 - 8.22 (m, 1H), 8.19 - 8.04 (m, 2H), 7.23 (d, J = 5.9 Hz, 1H), 6.76 (s, 1H), 4.07 - 3.97 (m, 1H), 3.83 (s, 3H), 3.47 (s, 3H), 3.44 - 3.38 (m, 2H), 3.29 - 3.25 (m, 1H), 3.12 (s, 3H), 3.10 - 3.05 (m, 1H), 3.03 - 2.94 (m, 6H), 2.56 - 2.33 (m, 2H), 2.31 - 2.14 (m, 2H), 0.93 - 0.60 (m, 3H). MS (ESI) m/z: 610 [M+H]⁺. |
| I-189 | 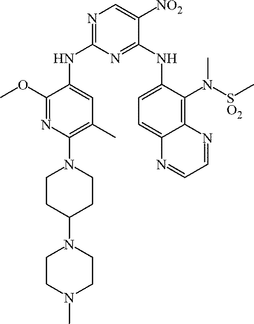 | ¹H NMR (600 MHz, Chloroform-d) δ 11.59 (s, 1H), 9.24 (s, 1H), 8.85 (s, 2H), 8.83 (d, J = 9.7 Hz, 1H), 8.50 (s, 1H), 7.97 (d, J = 9.3 Hz, 1H), 7.83 (s, 1H), 3.91 (s, 3H), 3.80 - 3.60 (m, 8H), 3.61 - 3.51 (m, 2H), 3.48 (s, 3H), 3.35 - 3.27 (m, 1H), 3.16 (s, 3H), 3.00 - 2.82 (m, 5H), 2.22 - 2.11 (m, 2H), 2.12 - 1.94 (m, 5H). MS (ESI) m/z: 693 [M+H]⁺. |
| I-190 | 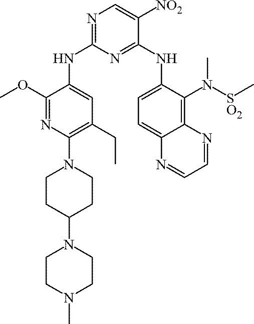 | ¹H NMR (600 MHz, DMSO-d₆) δ 11.19 (s, 1H), 9.94 (s, 1H), 9.17 (s, 1H), 9.01 (s, 1H), 8.96 (s, 1H), 8.52 (d, J = 9.4 Hz, 1H), 7.72 (d, J = 9.4 Hz, 1H), 7.57 (s, 1H), 3.83 (s, 3H), 3.68 - 3.50 (m, 8H), 3.47 - 3.43 (m, 2H), 3.37 (s, 3H), 3.28 - 3.26 (m, 1H), 3.22 (s, 3H), 2.93 - 2.89 (m, 2H), 2.87 (s, 3H), 2.47 - 2.34 (m, 2H), 2.18 - 2.09 (m, 2H), 1.86 - 1.76 (m, 2H), 0.92 (t, J = 7.5 Hz, 3H). MS (ESI) m/z: 707 [M+H]⁺. |
| I-191 | 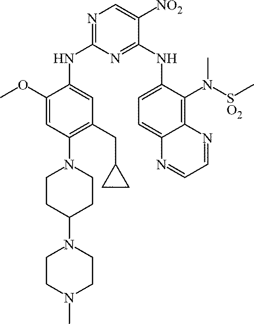 | ¹H NMR (600 MHz, Methanol-d₄) δ 9.13 - 9.05 (m, 1H), 8.94 (d, J = 1.9 Hz, 1H), 8.89 - 8.79 (m, 1H), 8.56 (d, J = 9.2 Hz, 1H), 7.80 (d, J = 9.3 Hz, 1H), 7.71 - 7.64 (m, 1H), 6.83 (s, 1H), 3.82 (s, 3H), 3.66 - 3.41 (m, 11H), 3.26 - 3.16 (m, 6H), 2.98 - 2.88 (m, 5H), 2.39 - 2.28 (m, 1H), 2.26 - 2.11 (m, 3H), 1.97 - 1.79 (m, 2H), 0.49 - 0.33 (m, 1H), 0.14 - 0.01 (m, 2H), -0.27 - -0.48 (m, 2H). MS (ESI) m/z: 732 [M+H]⁺. |
| I-192 | 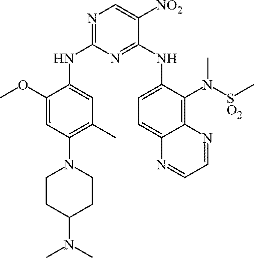 | ¹H NMR (600 MHz, DMSO-d₆) δ 10.08 (s, 1H), 9.85 (s, 1H), 9.14 (s, 1H), 8.98 (s, 1H), 8.93 (s, 1H), 8.64 (d, J = 9.4 Hz, 1H), 7.73 (d, J = 9.4 Hz, 1H), 7.20 (s, 1H), 6.82 (s, 1H), 3.77 (s, 3H), 3.36 (s, 3H), 3.29 - 3.23 (m, 3H), 3.20 (s, 3H), 2.88 - 2.83 (m, 6H), 2.80 - 2.71 (m, 2H), 2.20 - 2.13 (m, 2H), 2.08 (s, 3H), 1.92 - 1.77 (m, 2H). MS (ESI) m/z: 637 [M+H]⁺. |
| I-193 | 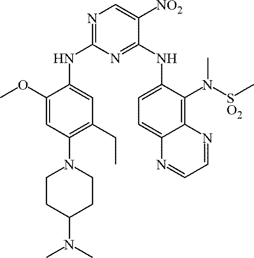 | ¹H NMR (600 MHz, Chloroform-d) δ 11.42 (s, 1H), 9.17 (s, 1H), 8.93 - 8.84 (m, 2H), 8.67 (d, J = 9.4 Hz, 1H), 8.17 (s, 1H), 8.00 (d, J = 9.4 Hz, 1H), 7.79 (s, 1H), 6.63 (s, 1H), 3.85 (s, 3H), 3.50 (s, 3H), 3.35 - 3.21 (m, 2H), 3.17 (s, 3H), 2.84 (s, 6H), 2.81 - 2.78 (m, 1H), 2.77 - 2.63 (m, 4H), 2.21 - 2.07 (m, 2H), 2.01 - 1.90 (m, 2H), 0.75 - 0.48 (m, 3H). MS (ESI) m/z: 651 [M+H]⁺. |
| I-194 | 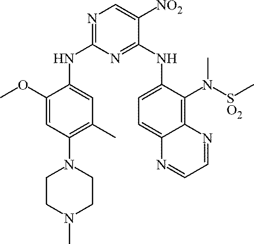 | ¹H NMR (600 MHz, Chloroform-d) δ 11.54 (s, 1H), 9.15 (s, 1H), 8.94 - 8.84 (m, 3H), 8.19 - 7.99 (m, 2H), 7.85 (s, 1H), 6.64 (s, 1H), 3.87 (s, 3H), 3.72 - 3.64 (m, 2H), 3.51 (s, 3H), 3.34 - 3.26 (m, 2H), 3.18 (s, 3H), 3.17 - 3.01 (m, 4H), 2.90 (s, 3H), 1.96 (s, 3H). MS (ESI) m/z: 609 [M+H]⁺. |
| I-195 | 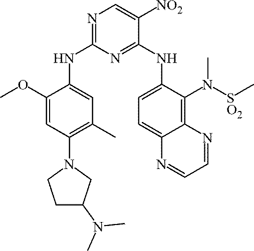 | ¹H NMR (600 MHz, Chloroform-d) δ 11.50 (s, 1H), 9.08 (d, J = 14.5 Hz, 1H), 8.96 - 8.78 (m, 3H), 8.12 (s, 1H), 8.07 - 8.00 (m, 1H), 7.76 (s, 1H), 6.57 (s, 1H), 3.90 - 3.80 (m, 3H), 3.50 (s, 3H), 3.43 - 3.32 (m, 2H), 3.18 (s, 3H), 3.11 - 3.04 (m, 1H), 2.96 - 2.88 (m, 6H), 2.82 - 2.68 (m, 2H), 2.45 - 2.37 (m, 1H), 2.35 - 2.26 (m, 1H), 2.01 - 1.92 (m, 3H). MS (ESI) m/z: 623 [M+H]⁺. |
| I-196 | 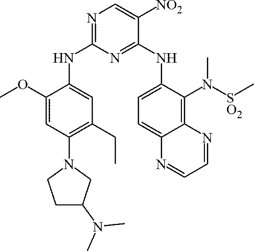 | ¹H NMR (600 MHz, Chloroform-d) δ 11.40 (d, J = 3.3 Hz, 1H), 9.12 (d, J = 6.8 Hz, 1H), 8.88 (d, J = 7.5 Hz, 2H), 8.77 - 8.62 (m, 1H), 8.22 (s, 1H), 8.05 - 7.97 (m, 1H), 7.73 (s, 1H), 6.67 (s, 1H), 3.90 - 3.80 (m, 3H), 3.49 (s, 3H), 3.43 - 3.29 (m, 4H), 3.16 (s, 3H), 3.12 - 3.06 (m, 1H), 2.90 (d, J = 8.6 Hz, 6H), 2.47 - 2.16 (m, 4H), 0.56 (t, J = 8.3 Hz, 3H). MS (ESI) m/z: 637 [M+H]⁺. |
| I-197 | 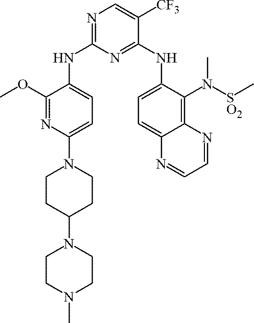 | ¹H NMR (600 MHz, Methanol-d₄) δ 8.99 - 8.94 (m, 1H), 8.94 - 8.88 (m, 1H), 8.50 - 8.20 (m, 2H), 8.07 - 7.79 (m, 1H), 7.52 (s, 1H), 6.43 - 6.07 (m, 1H), 4.56 - 4.36 (m, 2H), 3.86 (s, 3H), 3.66 - 3.49 (m, 8H), 3.46 - 3.43 (m, 1H), 3.42 (s, 3H), 3.14 (s, 3H), 2.94 (s, 3H), 2.93 - 2.89 (m, 2H), 2.23 - 2.10 (m, 2H), 1.80 - 1.65 (m, 2H). MS (ESI) m/z: 702 [M+H]⁺. |
| I-198 | 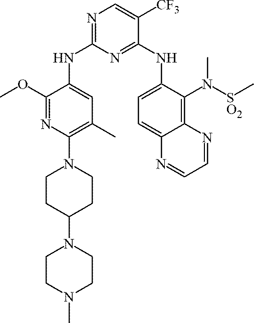 | ¹H NMR (600 MHz, Methanol-d₄) δ 9.00 - 8.93 (m, 1H), 8.91 - 8.83 (m, 1H), 8.51 - 8.38 (m, 1H), 7.94 - 7.75 (m, 1H), 7.45 (s, 1H), 3.93 (s, 3H), 3.83 - 3.70 (m, 2H), 3.66 - 3.48 (m, 8H), 3.46 - 3.39 (m, 3H), 3.33 - 3.28 (m, 1H), 3.17 (s, 3H), 2.93 (s, 3H), 2.92 - 2.82 (m, 2H), 2.05 (t, J = 16.1 Hz, 3H), 1.99 - 1.82 (m, 2H), 1.77 - 1.66 (m, 2H). MS (ESI) m/z: 716 [M+H]⁺. |
| I-199 | 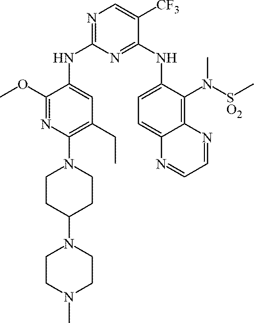 | ¹H NMR (600 MHz, Methanol-d₄) δ 8.98 (d, J = 1.9 Hz, 1H), 8.92 (d, J = 1.8 Hz, 1H), 8.51 - 8.21 (m, 2H), 8.12 - 7.93 (m, 1H), 7.87 - 7.66 (m, 1H), 3.92 (s, 3H), 3.74 - 3.57 (m, 8H), 3.51 - 3.47 (m, 2H), 3.43 (s, 3H), 3.37 - 3.33 (m, 1H), 3.14 (s, 3H), 3.03 - 2.99 (m, 2H), 2.97 (s, 3H), 2.39 - 2.17 (m, 4H), 2.06 - 1.89 (m, 2H), 1.02 - 0.34 (m, 3H). MS (ESI) m/z: 730 [M+H]⁺. |
| I-200 | 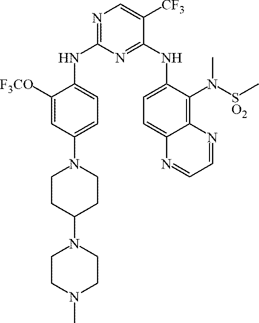 [SMILES: CN1CCN(C2CCN(c3ccc(Nc4ncc(C(F)(F)F)c(Nc5ccc6nccnc6c5N(C)S(C)(=O)=O)n4)c(OC(F)(F)F)c3)CC2)CC1] | ¹H NMR (600 MHz, Methanol-d₄) δ 8.94 (d, J = 1.9 Hz, 1H), 8.87 (d, J = 1.9 Hz, 1H), 8.57 - 8.45 (m, 1H), 8.39 (s, 1H), 7.96 - 7.85 (m, 1H), 7.43 (d, J = 8.8 Hz, 1H), 6.95 (s, 1H), 6.93 (d, J = 10.9 Hz, 1H), 3.94 - 3.82 (m, 2H), 3.66 - 3.47 (m, 8H), 3.40 (s, 3H), 3.30 - 3.26 (m, 1H), 3.15 (s, 3H), 2.96 (s, 3H), 2.90 (ddd, J = 12.7, 9.9, 3.0 Hz, 2H), 2.28-2.18 (m, 2H), 1.93 - 1.83 (m, 2H). MS (ESI) m/z: 755 [M+H]⁺. |
| I-201 | 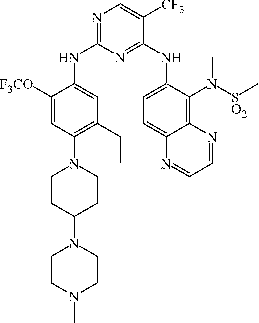 [SMILES: CCc1cc(Nc2ncc(C(F)(F)F)c(Nc3ccc4nccnc4c3N(C)S(C)(=O)=O)n2)c(OC(F)(F)F)cc1N1CCC(N2CCN(C)CC2)CC1] | ¹H NMR (600 MHz, Methanol-d₄) δ 8.96 (d, J = 1.8 Hz, 1H), 8.89 (d, J = 1.8 Hz, 1H), 8.44 (s, 1H), 8.40 - 8.21 (m, 1H), 7.99 - 7.79 (m, 1H), 7.44 (s, 1H), 7.05 (s, 1H), 3.77 - 3.61 (m, 8H), 3.41 (s, 3H), 3.40 - 3.36 (m, 1H), 3.20 - 3.16 (m, 2H), 3.14 (s, 3H), 3.00 (s, 3H), 2.90 - 2.67 (m, 2H), 2.55 - 2.39 (m, 2H), 2.33 - 2.15 (m, 2H), 2.07 - 1.88 (m, 2H), 1.03 - 0.71 (m, 3H). MS (ESI) m/z: 783 [M+H]⁺. |
| I-202 | 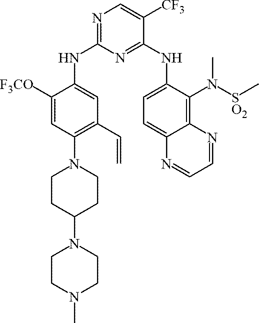 [SMILES: C=Cc1cc(Nc2ncc(C(F)(F)F)c(Nc3ccc4nccnc4c3N(C)S(C)(=O)=O)n2)c(OC(F)(F)F)cc1N1CCC(N2CCN(C)CC2)CC1] | ¹H NMR (600 MHz, Methanol-d₄) δ 8.95 (d, J = 1.8 Hz, 1H), 8.88 (d, J = 1.8 Hz, 1H), 8.44 (s, 1H), 8.39 - 8.17 (m, 1H), 7.87 - 7.75 (m, 1H), 7.68 (s, 1H), 6.99 (s, 1H), 6.88 - 6.67 (m, 1H), 5.43 - 5.04 (m, 2H), 3.82 - 3.60 (m, 8H), 3.42 (s, 3H), 3.39 - 3.34 (m, 2H), 3.34 - 3.32 (m, 1H), 3.14 (s, 3H), 3.00 (s, 3H), 2.83 - 2.70 (m, 2H), 2.33 - 2.19 (m, 2H), 2.07 - 1.90 (m, 2H). MS (ESI) m/z: 781 [M+H]⁺. |
| I-203 | 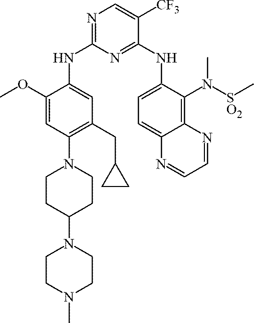 | ¹H NMR (600 MHz, Methanol-d₄) δ 8.95 (d, J = 1.9 Hz, 1H), 8.90 (d, J = 1.9 Hz, 1H), 8.40 (s, 2H), 7.95 (s, 1H), 7.55 (s, 1H), 6.80 (s, 1H), 3.81 (s, 3H), 3.67 - 3.47 (m, 8H), 3.40 (s, 3H), 3.31 - 3.28 (m, 1H), 3.19 - 3.11 (m, 2H), 3.09 (s, 3H), 2.93 (s, 3H), 2.91 - 2.81 (m, 2H), 2.30 - 1.98 (m, 4H), 1.96 - 1.79 (m, 2H), 0.51 - 0.23 (m, 1H), 0.20 - -0.08 (m, 2H), -0.02 - -0.69 (m, 2H). MS (ESI) m/z: 755 [M+H]⁺. |
| I-204 | 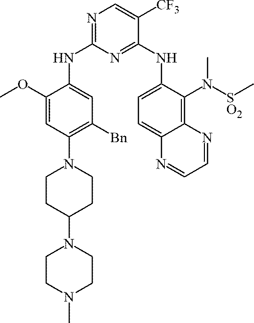 | ¹H NMR (600 MHz, Methanol-d₄) δ 8.89 - 8.84 (m, 1H), 8.81 - 8.77 (m, 1H), 8.48 - 8.28 (m, 2H), 8.05 - 7.87 (m, 1H), 7.57 (s, 1H), 7.07 - 6.90 (m, 3H), 6.83 (s, 1H), 6.80 - 6.49 (m, 2H), 3.84 (s, 3H), 3.57 - 3.40 (m, 8H), 3.35 (s, 3H), 3.17 - 3.12 (m, 1H), 3.10 (s, 3H), 3.04 - 2.99 (m, 1H), 2.91 (s, 3H), 2.88 - 2.81 (m, 1H), 2.81 - 2.64 (m, 2H), 2.13 - 1.95 (m, 2H), 1.84 - 1.65 (m, 2H). MS (ESI) m/z: 791 [M+H]⁺. |
| I-205 | 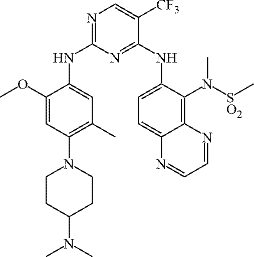 | ¹H NMR (600 MHz, Methanol-d₄) δ 8.98 (d, J = 1.9 Hz, 1H), 8.92 (d, J = 1.9 Hz, 1H), 8.58 - 8.27 (m, 2H), 8.12 - 7.93 (m, 1H), 7.33 (s, 1H), 6.74 (s, 1H), 3.82 (s, 3H), 3.43 (s, 3H), 3.38 - 3.34 (m, 1H), 3.28 - 3.20 (m, 2H), 3.14 (s, 3H), 2.94 (s, 6H), 2.85 - 2.73 (m, 2H), 2.26 - 2.16 (m, 2H), 2.09 - 1.70 (m, 5H). MS (ESI) m/z: 660 [M+H]⁺. |
| I-206 | 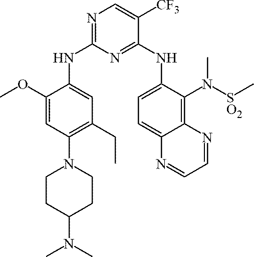 | ¹H NMR (600 MHz, Methanol-d₄) δ 8.98 (d, J = 1.8 Hz, 1H), 8.92 (d, J = 1.8 Hz, 1H), 8.62 - 8.26 (m, 2H), 8.10 - 7.86 (m, 1H), 7.40 (s, 1H), 6.79 (s, 1H), 3.83 (s, 3H), 3.43 (s, 3H), 3.37 - 3.33 (m, 1H), 3.21 - 3.09 (m, 5H), 2.99 - 2.91 (m, 6H), 2.82 (t, J = 11.0 Hz, 2H), 2.45 - 2.10 (m, 4H), 2.00 - 1.83 (m, 2H), 1.07 - 0.25 (m, 3H). MS (ESI) m/z: 674 [M+H]⁺. |
| I-207 | 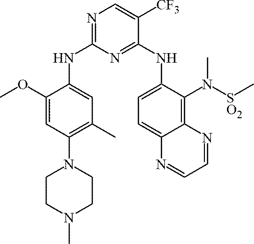 | ¹H NMR (600 MHz, Methanol-d₄) δ 8.97 (d, J = 1.9 Hz, 1H), 8.90 (d, J = 1.9 Hz, 1H), 8.60 - 8.45 (m, 1H), 8.40 (s, 1H), 8.05 - 7.93 (m, 1H), 7.46 (s, 1H), 6.76 (s, 1H), 3.84 (s, 3H), 3.64 - 3.54 (m, 2H), 3.43 (s, 3H), 3.36 - 3.32 (m, 2H), 3.28 - 3.20 (m, 2H), 3.15 (s, 3H), 3.12 - 3.05 (m, 2H), 2.99 (s, 3H), 2.10 - 1.74 (m, 3H). MS (ESI) m/z: 632 [M+H]⁺. |
| I-208 | 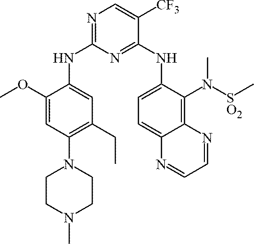 | ¹H NMR (600 MHz, Methanol-d₄) δ 8.97 (d, J = 1.9 Hz, 1H), 8.91 (d, J = 1.9 Hz, 1H), 8.51 - 8.29 (m, 2H), 8.06 - 7.89 (m, 1H), 7.51 (s, 1H), 6.81 (s, 1H), 3.85 (s, 3H), 3.67 - 3.54 (m, 2H), 3.44 (s, 3H), 3.31 - 3.26 (m, 2H), 3.21 - 3.11 (m, 7H), 2.99 (s, 3H), 2.58 - 2.08 (m, 2H), 1.14 - 0.33 (m, 3H). MS (ESI) m/z: 646 [M+H]⁺. |
| I-209 | 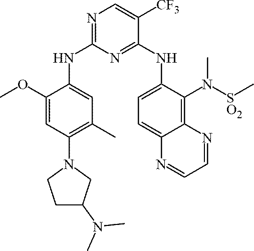 | ¹H NMR (600 MHz, Methanol-d₄) δ 8.98 (d, J = 1.8 Hz, 1H), 8.92 (d, J = 1.7 Hz, 1H), 8.56 - 8.24 (m, 2H), 8.08 - 7.87 (m, 1H), 7.27 (s, 1H), 6.70 (s, 1H), 4.08 - 3.99 (m, 1H), 3.83 (s, 3H), 3.45 (s, 3H), 3.42 - 3.33 (m, 3H), 3.14 (s, 4H), 3.05 - 2.95 (m, 6H), 2.57 - 2.44 (m, 1H), 2.32 - 2.19 (m, 1H), 2.13 - 1.59 (m, 3H). MS (ESI) m/z: 646 [M+H]⁺. |
| I-210 | 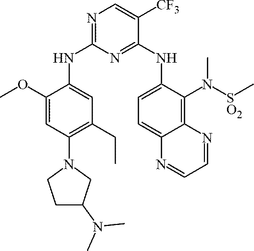 | ¹H NMR (600 MHz, Methanol-d₄) δ 8.98 (d, J = 1.9 Hz, 1H), 8.93 (d, J = 1.8 Hz, 1H), 8.54 - 8.39 (m, 1H), 8.37 - 8.20 (m, 1H), 8.11 - 7.86 (m, 1H), 7.33 (s, 1H), 6.78 (s, 1H), 4.08 - 3.96 (m, 1H), 3.84 (s, 3H), 3.59 - 3.38 (m, 6H), 3.13 (s, 3H), 3.11 - 3.06 (m, 1H), 2.98 (s, 6H), 2.62 - 2.09 (m, 4H), 1.16 - 0.39 (m, 3H). MS (ESI) m/z: 660 [M+H]⁺. |
| I-211 | 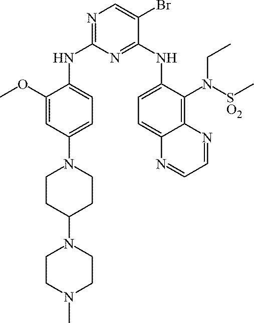 | ¹H NMR (600 MHz, Methanol-d₄) δ 8.97 (d, J = 1.9 Hz, 1H), 8.90 (d, J = 1.9 Hz, 1H), 8.80 (d, J = 9.4 Hz, 1H), 8.30 (s, 1H), 8.11 (d, J = 9.4 Hz, 1H), 7.86 (d, J = 8.6 Hz, 1H), 7.06 (d, J = 2.4 Hz, 1H), 6.89 (dd, J = 8.9, 2.5 Hz, 1H), 4.17 (dq, J = 14.6, 7.3 Hz, 1H), 3.93 (s, 3H), 3.91 - 3.81 (m, 3H), 3.45 (s, 4H), 3.38 - 3.35 (m, 2H), 3.21 (s, 7H), 3.13 - 3.07 (m, 1H), 2.94 (s, 3H), 2.31 - 2.19 (m, 2H), 2.02 (qd, J = 12.2, 3.9 Hz, 2H), 1.04 (t, J = 7.2 Hz, 3H). MS (ESI) m/z: 725 [M+H]⁺. |
| I-212 | 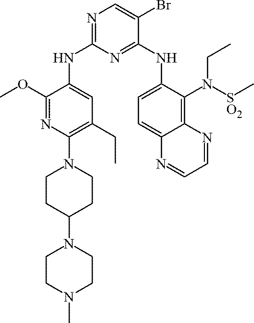 | ¹H NMR (600 MHz, Methanol-d₄) δ 8.93 (d, J = 1.8 Hz, 1H), 8.87 (d, J = 1.9 Hz, 1H), 8.62 - 8.47 (m, 1H), 8.26 (s, 1H), 7.99 (d, J = 9.3 Hz, 1H), 7.80 (s, 1H), 4.17 - 4.06 (m, 1H), 3.90 (s, 3H), 3.82 - 3.75 (m, 1H), 3.64 - 3.49 (m, 10H), 3.28 (p, J = 1.6 Hz, 1H), 3.13 (s, 3H), 3.05 - 2.98 (m, 2H), 2.93 (s, 3H), 2.49 - 2.32 (m, 2H), 2.22 - 2.13 (m, 2H), 1.99 - 1.88 (m, 2H), 0.97 (t, J = 7.1 Hz, 3H), 0.89 - 0.73 (m, 3H). MS (ESI) m/z: 754 [M+H]⁺. |
| I-213 | 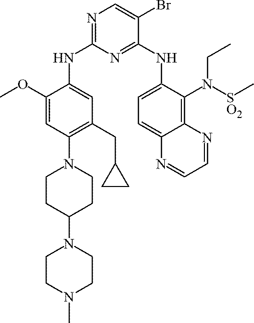 | ¹H NMR (600 MHz, Methanol-d₄) δ 8.94 (d, J = 1.9 Hz, 1H), 8.88 (d, J = 1.8 Hz, 1H), 8.71 - 8.58 (m, 1H), 8.24 (s, 1H), 7.98 (d, J = 9.4 Hz, 1H), 7.73 (s, 1H), 6.83 (s, 1H), 4.13 (dq, J = 14.5, 7.3 Hz, 1H), 3.84 (s, 3H), 3.77 (dq, J = 13.9, 7.0 Hz, 1H), 3.46 - 3.29 (m, 8H), 3.23 - 3.18 (m, 2H), 3.14 (s, 3H), 3.08 - 3.00 (m, 1H), 2.97 - 2.86 (m, 2H), 2.85 (s, 3H), 2.47 (dd, J = 14.6, 6.7 Hz, 1H), 2.28 - 2.16 (m, 1H), 2.16 - 2.07 (m, 2H), 1.94 - 1.74 (m, 2H), 1.01 (t, J = 7.2 Hz, 3H), 0.57 - 0.48 (m, 1H), 0.20 - 0.00 (m, 2H), -0.12 - -0.32 (m, 2H). MS (ESI) m/z: 779 [M+H]⁺. |
| I-214 | 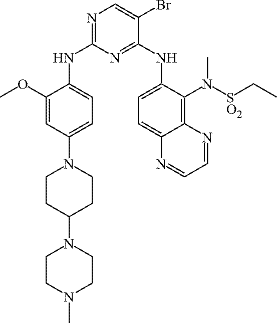 [SMILES: CCS(=O)(=O)N(C)c1c(Nc2nc(Nc3ccc(N4CCC(N5CCN(C)CC5)CC4)cc3OC)ncc2Br)ccc2nccnc12] | ¹H NMR (600 MHz, Methanol-d₄) δ 8.97 (d, J = 1.9 Hz, 1H), 8.89 (d, J = 1.8 Hz, 1H), 8.68 (d, J = 9.4 Hz, 1H), 8.26 (s, 1H), 8.08 (d, J = 9.3 Hz, 1H), 7.75 (d, J = 8.8 Hz, 1H), 7.01 (d, J = 2.4 Hz, 1H), 6.86 - 6.75 (m, 1H), 3.90 (s, 3H), 3.87 - 3.80 (m, 2H), 3.58 - 3.34 (m, 9H), 3.30 - 3.15 (m, 6H), 3.12 - 3.04 (m, 1H), 2.93 (s, 3H), 2.28 - 2.17 (m, 2H), 2.04 - 1.93 (m, 2H), 1.43 (t, J = 7.4 Hz, 3H). MS (ESI) m/z: 725 [M+H]⁺. |
| I-215 | 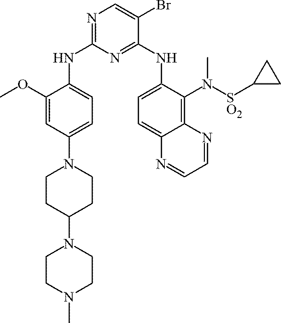 [SMILES: COc1cc(N2CCC(N3CCN(C)CC3)CC2)ccc1Nc1ncc(Br)c(Nc2ccc3nccnc3c2N(C)S(=O)(=O)C2CC2)n1] | ¹H NMR (600 MHz, Methanol-d₄) δ 8.95 (d, J = 1.9 Hz, 1H), 8.86 (d, J = 1.9 Hz, 1H), 8.65 (d, J = 9.5 Hz, 1H), 8.23 (s, 1H), 8.05 (d, J = 9.3 Hz, 1H), 7.70 (d, J = 8.9 Hz, 1H), 6.97 (d, J = 2.5 Hz, 1H), 6.78 (dd, J = 8.9, 2.5 Hz, 1H), 3.87 (s, 3H), 3.84 - 3.75 (m, 2H), 3.49 (s, 3H), 3.47 - 3.34 (m, 4H), 3.28 - 3.15 (m, 6H), 3.13 - 3.05 (m, 1H), 2.91 (s, 3H), 2.87 (td, J = 8.0, 4.0 Hz, 1H), 2.30 - 2.14 (m, 2H), 2.02 - 1.87 (m, 2H), 1.23 - 1.07 (m, 2H), 0.75 - 0.63 (m, 2H). MS (ESI) m/z: 737 [M+H]⁺. |
| I-216 | 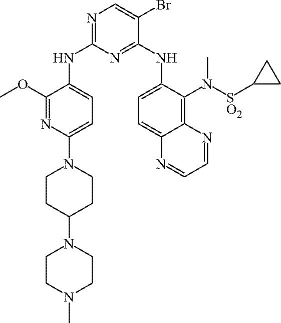 [SMILES: COc1nc(N2CCC(N3CCN(C)CC3)CC2)ccc1Nc1ncc(Br)c(Nc2ccc3nccnc3c2N(C)S(=O)(=O)C2CC2)n1] | ¹H NMR (600 MHz, Methanol-d₄) δ 8.97 (d, J = 1.9 Hz, 1H), 8.88 (d, J = 1.8 Hz, 1H), 8.68 (m, 1H), 8.20 (s, 1H), 8.10 - 7.96 (m, 1H), 7.56 (d, J = 8.5 Hz, 1H), 6.32 (d, J = 8.7 Hz, 1H), 4.53 - 4.44 (m, 2H), 3.87 (s, 3H), 3.51 (s, 3H), 3.29 - 3.11 (m, 6H), 3.09 - 3.04 (m, 1H), 2.97 - 2.86 (m, 5H), 2.83 (s, 3H), 2.16 - 2.04 (m, 2H), 1.72 - 1.61 (m, 2H), 1.23 - 1.16 (m, 2H), 0.77 - 0.62 (m, 2H). MS (ESI) m/z: 738 [M+H]⁺. |
| I-217 | 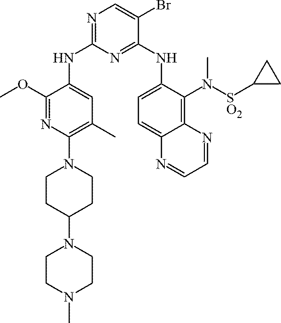 [SMILES: COc1nc(N2CCC(N3CCN(C)CC3)CC2)c(C)cc1Nc1ncc(Br)c(Nc2ccc3nccnc3c2N(C)S(=O)(=O)C2CC2)n1] | ¹H NMR (600 MHz, Methanol-d₄) δ 8.94 (d, J = 1.8 Hz, 1H), 8.86 (d, J = 1.8 Hz, 1H), 8.63 (s, 1H), 8.23 (s, 1H), 8.00 - 7.85 (m, 1H), 7.45 (s, 1H), 3.93 (s, 3H), 3.83 - 3.70 (m, 2H), 3.51 (s, 3H), 3.46 - 3.30 (m, 8H), 3.16 - 3.07 (m, 1H), 2.96 - 2.83 (m, 6H), 2.11 - 1.94 (m, 5H), 1.72 - 1.57 (m, 2H), 1.28 - 1.13 (m, 2H), 0.78 - 0.60 (m, 2H). MS (ESI) m/z: 752 [M+H]⁺. |
| I-218 | 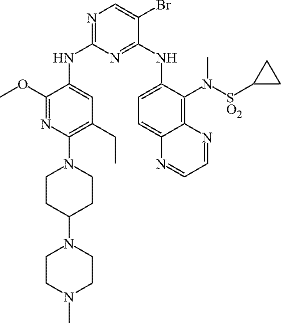 [SMILES: CCc1cc(Nc2ncc(Br)c(Nc3ccc4nccnc4c3N(C)S(=O)(=O)C3CC3)n2)c(OC)nc1N1CCC(N2CCN(C)CC2)CC1] | ¹H NMR (600 MHz, Methanol-d₄) δ 8.98 (d, J = 1.9 Hz, 1H), 8.90 (d, J = 1.9 Hz, 1H), 8.59 - 8.45 (m, 1H), 8.27 (s, 1H), 8.02 (d, J = 9.3 Hz, 1H), 7.77 (s, 1H), 3.93 (s, 3H), 3.68 - 3.41 (m, 13H), 3.29 (d, J = 14.8 Hz, 1H), 3.01 (t, J = 12.6 Hz, 2H), 2.94 (s, 3H), 2.89 - 2.84 (m, 1H), 2.49 -2.33 (m, 2H), 2.24 - 2.14 (m, 2H), 1.94 (dt, J = 12.1, 3.0 Hz, 2H), 1.22 - 1.13 (m, 2H), 0.97 - 0.77 (m, 3H), 0.76 - 0.63 (m, 2H). MS (ESI) m/z: 766 [M+H]⁺. |
| I-219 | 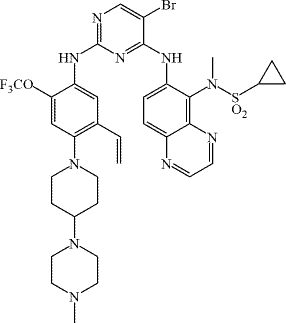 [SMILES: C=Cc1cc(Nc2ncc(Br)c(Nc3ccc4nccnc4c3N(C)S(=O)(=O)C3CC3)n2)c(OC(F)(F)F)cc1N1CCC(N2CCN(C)CC2)CC1] | ¹H NMR (600 MHz, Methanol-d₄) δ 8.94 (d, J = 1.9 Hz, 1H), 8.83 (d, J = 1.9 Hz, 1H), 8.60 - 8.52 (m, 1H), 8.27 (s, 1H), 7.83 (d, J = 9.5 Hz, 1H), 7.77 (s, 1H), 7.01 (s, 1H), 6.88 (dd, J = 17.7, 11.0 Hz, 1H), 5.39 (d, J = 17.8 Hz, 1H), 5.02 - 5.01 (m, 1H), 3.48 (s, 3H), 3.45 - 3.35 (m, 8H), 3.10 - 3.03 (m, 1H), 2.89 (d, J = 4.9 Hz, 6H), 2.74 (t, J = 11.9 Hz, 2H), 2.18 - 2.08 (m, 2H), 1.95 - 1.84 (m, 2H), 1.24 - 1.12 (m, 2H), 0.76 - 0.63 (m, 2H). MS (ESI) m/z: 817 [M+H]⁺. |
| I-220 | 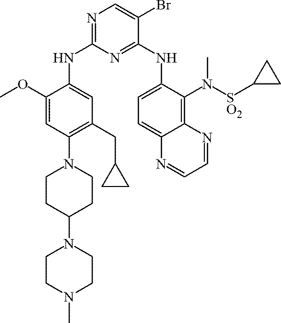 [SMILES: COc1cc(N2CCC(N3CCN(C)CC3)CC2)c(CC2CC2)cc1Nc1ncc(Br)c(Nc2ccc3nccnc3c2N(C)S(=O)(=O)C2CC2)n1] | ¹H NMR (600 MHz, Methanol-d₄) δ 8.95 (d, J = 1.9 Hz, 1H), 8.87 (d, J = 1.9 Hz, 1H), 8.62 - 8.39 (m, 1H), 8.22 (s, 1H), 7.97 (d, J = 9.1 Hz, 1H), 7.60 (s, 1H), 6.82 (s, 1H), 3.82 (s, 3H), 3.53 - 3.39 (m, 11H), 3.21 - 3.13 (m, 3H), 2.90 - 2.88 (m, 3H), 2.88 - 2.80 (m, 3H), 2.39 - 2.32 (m, 1H), 2.23 (s, 1H), 2.19 - 2.08 (m, 2H), 1.94 - 1.81 (m, 2H), 1.21 - 1.11 (m, 2H), 0.73 - 0.63 (m, 2H), 0.63 - 0.46 (m, 1H), 0.30 - 0.11 (m, 2H), -0.04 - -0.36 (m, 2H). MS (ESI) m/z: 791 [M+H]⁺. |
| I-221 | 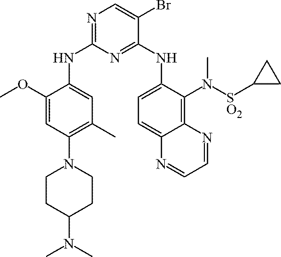 [SMILES: COc1cc(N2CCC(N(C)C)CC2)c(C)cc1Nc1ncc(Br)c(Nc2ccc3nccnc3c2N(C)S(=O)(=O)C2CC2)n1] | ¹H NMR (600 MHz, Methanol-d4) δ 8.95 (d, J = 1.8 Hz, 1H), 8.88 (d, J = 1.9 Hz, 1H), 8.65 - 8.38 (m, 1H), 8.29 - 8.14 (m, 1H), 8.06 - 7.91 (m, 1H), 7.24 (s, 1H), 6.73 (s, 1H), 3.79 (s, 3H), 3.48 (s, 3H), 3.33 - 3.30 (m, 1H), 3.25 - 3.19 (m, 2H), 2.91 (s, 6H), 2.87 - 2.82 (m, 1H), 2.79 - 2.68 (m, 2H), 2.25 - 2.12 (m, 2H), 1.99 (brs, 3H), 1.94 - 1.82 (m, 2H), 1.23 - 1.08 (m, 2H), 0.75 - 0.54 (m, 2H). MS (ESI) m/z: 696 [M+H]+. |
| I-222 | 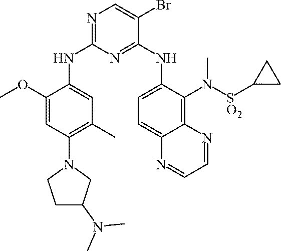 [SMILES: COc1cc(N2CCC(N(C)C)C2)c(C)cc1Nc1ncc(Br)c(Nc2ccc3nccnc3c2N(C)S(=O)(=O)C2CC2)n1] | ¹H NMR (600 MHz, Methanol-d₄) δ 8.98 (d, J = 1.9 Hz, 1H), 8.89 (d, J = 1.8 Hz, 1H), 8.68 - 8.54 (m, 1H), 8.23 (s, 1H), 8.01 (d, J = 9.3 Hz, 1H), 7.33 (s, 1H), 6.72 (d, J = 1.6 Hz, 1H), 4.08 - 3.99 (m, 1H), 3.84 (s, 3H), 3.51 (s, 3H), 3.50 - 3.46 (m, 1H), 3.45 - 3.36 (m, 2H), 3.18 - 3.08 (m, 1H), 2.99 (s, 6H), 2.92 - 2.83 (m, 1H), 2.54 - 2.46 (m, 1H), 2.29 - 2.18 (m, 1H), 2.07 (br s, 3H), 1.24 - 1.13 (m, 2H), 0.77 - 0.63 (m, 2H). MS (ESI) m/z: 682 [M+H]⁺. |
| I-223 | 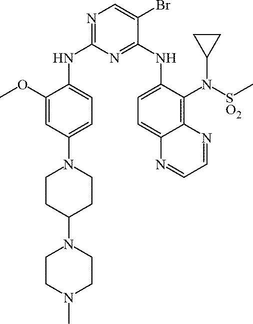 | ¹H NMR (600 MHz, Methanol-d₄) δ 8.95 - 8.93 (m, 1H), 8.83 - 8.81 (m, 1H), 8.79 (d, J = 9.5 Hz, 1H), 8.21 (d, J = 2.0 Hz, 1H), 7.97 (dd, J = 9.4, 2.0 Hz, 1H), 7.86 (d, J = 7.8 Hz, 1H), 7.05 (s, 1H), 6.88 (d, J = 8.4 Hz, 1H), 3.90 (s, 3H), 3.85 - 3.80 (m, 2H), 3.73 - 3.67 (m, 1H), 3.57 - 3.32 (m, 10H), 3.28 - 3.12 (m, 3H), 3.11 - 3.04 (m, 1H), 2.93 (s, 3H), 2.28 - 2.17 (m, 2H), 2.02 (td, J = 14.3, 7.1 Hz, 2H), 1.32 - 1.21 (m, 1H), 1.00 (ddd, J = 9.2, 6.2, 2.3 Hz, 1H), 0.42 (ddd, J = 9.6, 5.1, 2.2 Hz, 1H), 0.33 - 0.20 (m, 1H). MS (ESI) m/z: 737 [M+H]⁺. |
| I-224 | 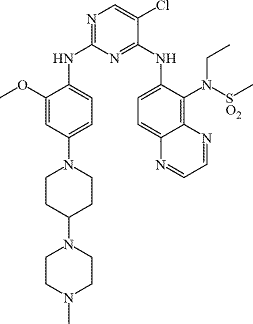 | ¹H NMR (600 MHz, Methanol-d₄) δ 8.93 (d, J = 1.9 Hz, 1H), 8.87 - 8.81 (m, 1H), 8.74 (d, J = 9.4 Hz, 1H), 8.18 (s, 1H), 8.03 (d, J = 9.3 Hz, 1H), 7.78 (d, J = 8.8 Hz, 1H), 7.03 (d, J = 2.5 Hz, 1H), 6.86 (dd, J = 8.9, 2.5 Hz, 1H), 4.14 (dq, J = 14.6, 7.4 Hz, 1H), 3.90 (s, 3H), 3.88 - 3.83 (m, 2H), 3.83 - 3.77 (m, 1H), 3.63 - 3.48 (m, 4H), 3.45 - 3.31 (m, 6H), 3.27 - 3.23 (m, 1H), 3.20 (s, 3H), 2.97 (s, 3H), 2.35 - 2.22 (m, 2H), 2.06 (td, J = 12.3, 3.9 Hz, 2H), 1.06 - 0.99 (m, 3H). MS (ESI) m/z: 681 [M+H]⁺. |
| I-225 | 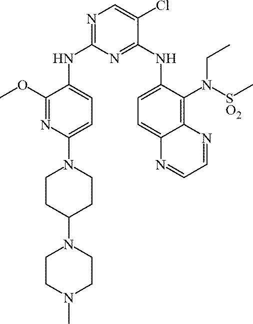 | ¹H NMR (600 MHz, Methanol-d₄) δ 8.96 (d, J = 1.8 Hz, 1H), 8.90 (d, J = 1.8 Hz, 1H), 8.79 (d, J = 41.4 Hz, 1H), 8.18 (s, 1H), 8.04 (s, 1H), 7.68 - 7.59 (m, 1H), 6.38 (d, J = 8.5 Hz, 1H), 4.61 - 4.50 (m, 2H), 4.23 - 4.10 (m, 1H), 3.89 (s, 3H), 3.86 - 3.80 (m, 1H), 3.69 - 3.48 (m, 8H), 3.45 - 3.41 (m, 1H), 3.19 (s, 3H), 3.03 - 2.93 (m, 5H), 2.31 - 2.10 (m, 2H), 1.86 - 1.71 (m, 2H), 1.03 (t, J = 7.2 Hz, 3H). MS (ESI) m/z: 682 [M+H]⁺. |
| I-226 | 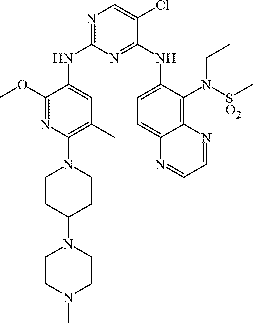 | ¹H NMR (600 MHz, Methanol-d₄) δ 8.95 (d, J = 1.9 Hz, 1H), 8.89 (d, J = 1.8 Hz, 1H), 8.84 - 8.70 (m, 1H), 8.21 (s, 1H), 8.09 - 7.92 (m, 1H), 7.52 (s, 1H), 4.19 - 4.08 (m, 1H), 3.97 (s, 3H), 3.90 - 3.78 (m, 3H), 3.56 - 3.37 (m, 8H), 3.23 - 3.17 (m, 4H), 2.96 - 2.87 (m, 5H), 2.07 (s, 3H), 2.02 (d, J = 18.1 Hz, 2H), 1.78 - 1.61 (m, 2H), 1.04 (t, J = 7.1 Hz, 3H). MS (ESI) m/z: 696 [M+H]⁺. |
| I-227 | 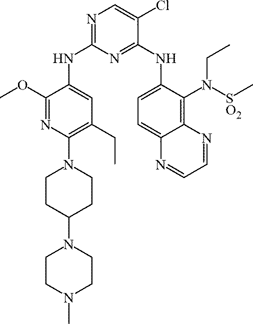 | ¹H NMR (600 MHz, Methanol-d₄) δ 8.96 (d, J = 1.9 Hz, 1H), 8.89 (d, J = 1.9 Hz, 1H), 8.71 - 8.55 (m, 1H), 8.21 (s, 1H), 8.03 (d, J = 9.3 Hz, 1H), 7.88 (s, 1H), 4.13 (dq, J = 14.5, 7.3 Hz, 1H), 3.94 (s, 3H), 3.81 (dq, J = 13.9, 7.0 Hz, 1H), 3.69 - 3.48 (m, 10H), 3.35 - 3.32 (m, 1H), 3.17 (s, 3H), 3.05 (q, J = 11.9 Hz, 2H), 2.96 (s, 3H), 2.55 - 2.45 (m, 1H), 2.45 - 2.38 (m, 1H), 2.26 - 2.13 (m, 2H), 2.05 - 1.91 (m, 2H), 1.00 (t, J = 7.2 Hz, 3H), 0.90 - 0.74 (m, 3H). MS (ESI) m/z: 710 [M+H]⁺. |
| I-228 | 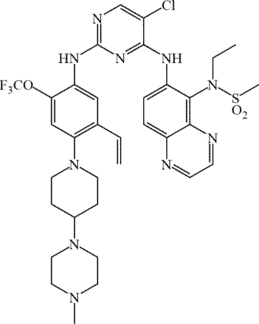 [SMILES: C=Cc1cc(Nc2ncc(Cl)c(Nc3ccc4nccnc4c3N(CC)S(C)(=O)=O)n2)c(OC(F)(F)F)cc1N1CCC(N2CCN(C)CC2)CC1] | ¹H NMR (600 MHz, Methanol-d₄) δ 8.94 (d, J = 1.9 Hz, 1H), 8.88 (d, J = 1.8 Hz, 1H), 8.60 - 8.42 (m, 1H), 8.25 (s, 1H), 7.86 (d, J = 9.4 Hz, 1H), 7.78 (s, 1H), 7.05 - 6.99 (m, 1H), 6.92 (dd, J = 17.7, 11.1 Hz, 1H), 5.43 (d, J = 17.6 Hz, 1H), 5.07 (d, J = 11.1 Hz, 1H), 4.18 - 4.08 (m, 1H), 3.82 - 3.77 (m, 1H), 3.74 - 3.58 (m, 8H), 3.42 - 3.37 (m, 2H), 3.35 - 3.32 (m, 1H), 3.15 (s, 3H), 3.00 (s, 3H), 2.83 - 2.71 (m, 2H), 2.34 - 2.20 (m, 2H), 2.09 - 1.93 (m, 2H), 0.99 (t, J = 7.2 Hz, 3H). MS (ESI) m/z: 761 [M+H]⁺. |
| I-229 | 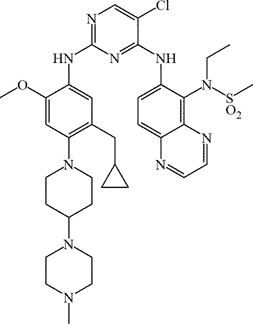 | ¹H NMR (600 MHz, Methanol-d₄) δ 8.94 (d, J = 1.8 Hz, 1H), 8.88 (d, J = 1.9 Hz, 1H), 8.68 - 8.47 (m, 1H), 8.18 (s, 1H), 7.99 (d, J = 9.5 Hz, 1H), 7.68 (s, 1H), 6.84 (s, 1H), 4.12 (dq, J = 14.5, 7.3 Hz, 1H), 3.83 (s, 3H), 3.76 (dq, J = 13.8, 6.9 Hz, 1H), 3.64 - 3.39 (m, 8H), 3.25 - 3.17 (m, 3H), 3.13 (s, 3H), 2.99 - 2.85 (m, 5H), 2.46 (dd, J = 14.7, 6.7 Hz, 1H), 2.30-2.21 (m, 1H), 2.20 - 2.12 (m, 2H), 1.98 - 1.83 (m, 2H), 0.98 (t, J = 7.2 Hz, 3H), 0.56 (s, 1H), 0.25 - 0.04 (m, 2H), -0.13 - -0.32 (m, 2H). MS (ESI) m/z: 735 [M+H]⁺. |
| I-230 | 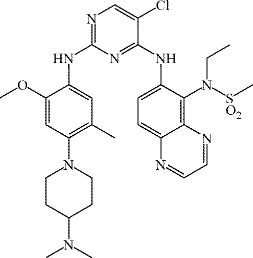 | ¹H NMR (600 MHz, Methanol-d₄) δ 8.93 (d, J = 1.9 Hz, 1H), 8.86 (d, J = 1.8 Hz, 1H), 8.80 - 8.68 (m, 1H), 8.15 (s, 1H), 7.99 (d, J = 9.3 Hz, 1H), 7.50 (s, 1H), 6.75 (s, 1H), 4.17 - 4.08 (m, 1H), 3.83 (s, 3H), 3.82 - 3.78 (m, 1H), 3.35 - 3.33 (m, 1H), 3.30 - 3.22 (m, 2H), 3.17 (s, 3H), 2.94 (s, 6H), 2.86 - 2.75 (m, 2H), 2.28 - 2.16 (m, 2H), 2.09 (s, 3H), 1.99 - 1.88 (m, 2H), 1.01 (t, J = 7.2 Hz, 3H). MS (ESI) m/z: 640 [M+H]⁺. |
| I-231 | 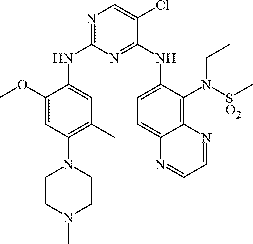 | ¹H NMR (600 MHz, Methanol-d₄) δ 8.95 (d, J = 1.9 Hz, 1H), 8.87 (d, J = 1.8 Hz, 1H), 8.72 (d, J = 9.1 Hz, 1H), 8.17 (s, 1H), 8.01 (d, J = 9.3 Hz, 1H), 7.56 (s, 1H), 6.78 (s, 1H), 4.20 - 4.07 (m, 1H), 3.85 (s, 3H), 3.84 - 3.79 (m, 1H), 3.65 - 3.58 (m, 2H), 3.34 - 3.31 (m, 2H), 3.30 - 3.23 (m, 2H), 3.17 (s, 3H), 3.15 - 3.08 (m, 2H), 3.00 (s, 3H), 2.10 (s, 3H), 1.00 (t, J = 7.0 Hz, 3H). MS (ESI) m/z: 612 [M+H]⁺. |
| I-232 | 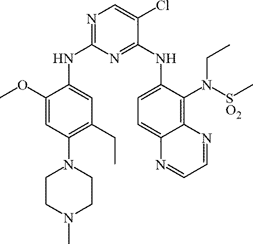 | ¹H NMR (600 MHz, DMSO-d₆) δ 10.25 (s, 1H), 8.99 (d, J = 1.8 Hz, 1H), 8.95 (d, J = 1.9 Hz, 1H), 8.87 (s, 1H), 8.64 (s, 1H), 8.43 (s, 1H), 8.29 (s, 1H), 8.00 (d, J = 9.4 Hz, 1H), 7.53 (s, 1H), 6.82 (s, 1H), 4.07 (dq, J = 14.4, 7.3 Hz, 1H), 3.82 (s, 3H), 3.69 (dq, J = 13.9, 7.0 Hz, 1H), 3.60 - 3.51 (m, 2H), 3.31 - 3.20 (m, 5H), 3.19 - 3.04 (m, 4H), 2.92 (s, 3H), 2.60 - 2.53 (m, 1H), 2.51 - 2.43 (m, 1H), 1.07 - 0.97 (m, 3H), 0.95 - 0.78 (m, 3H). MS (ESI) m/z: 626 [M+H]⁺. |
| I-233 | 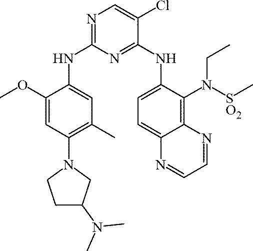 | ¹H NMR (600 MHz, Methanol-d₄) δ 8.96 (d, J = 1.8 Hz, 1H), 8.90 (d, J = 1.9 Hz, 1H), 8.78 - 8.52 (m, 1H), 8.19 (s, 1H), 8.07 - 7.95 (m, 1H), 7.34 (d, J = 2.2 Hz, 1H), 6.73 (d, J = 2.6 Hz, 1H), 4.20 - 4.10 (m, 1H), 4.08 - 3.99 (m, 1H), 3.84 (s, 3H), 3.83 - 3.79 (m, 1H), 3.56 - 3.48 (m, 1H), 3.47 - 3.38 (m, 2H), 3.22 - 3.13 (m, 4H), 3.00 (s, 6H), 2.58 - 2.48 (m, 1H), 2.28 - 2.20 (m, 1H), 2.14 (d, J = 7.0 Hz, 3H), 1.00 (t, J = 7.2 Hz, 3H). MS (ESI) m/z: 626 [M+H]⁺. |
| I-234 | 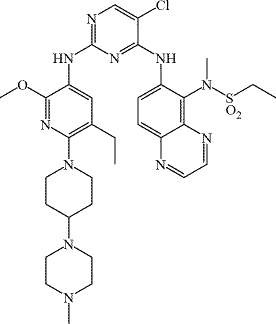 [SMILES: CCc1cc(Nc2ncc(Cl)c(Nc3ccc4nccnc4c3N(C)S(=O)(=O)CC)n2)c(OC)nc1N1CCC(N2CCN(C)CC2)CC1] | ¹H NMR (600 MHz, Methanol-d₄) δ 8.99 (d, J = 1.9 Hz, 1H), 8.92 (d, J = 1.9 Hz, 1H), 8.52 - 8.35 (m, 1H), 8.21 (s, 1H), 8.04 (d, J = 9.3 Hz, 1H), 7.76 (s, 1H), 3.93 (s, 3H), 3.66 - 3.53 (m, 8H), 3.51 - 3.48 (m, 2H), 3.46 (s, 3H), 3.36 - 3.31 (m, 2H), 3.25 - 3.11 (m, 1H), 3.06 - 2.98 (m, 2H), 2.96 (s, 3H), 2.43 - 2.26 (m, 2H), 2.26 - 2.16 (m, 2H), 2.02 - 1.88 (m, 2H), 1.41 (t, J = 7.4 Hz, 3H), 0.93 - 0.68 (m, 3H). MS (ESI) m/z: 710 [M+H]⁺. |
| I-235 | 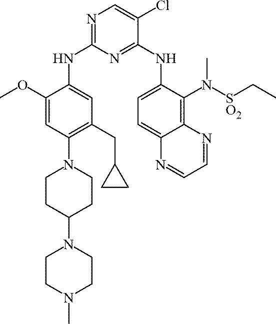 [SMILES: CCS(=O)(=O)N(C)c1c(Nc2nc(Nc3cc(CC4CC4)c(N4CCC(N5CCN(C)CC5)CC4)cc3OC)ncc2Cl)ccc2nccnc12] | ¹H NMR (600 MHz, Methanol-d₄) δ 9.00 (d, J = 1.9 Hz, 1H), 8.94 (d, J = 1.8 Hz, 1H), 8.45 (s, 1H), 8.21 (s, 1H), 8.04 (d, J = 9.2 Hz, 1H), 7.58 (s, 1H), 6.85 (s, 1H), 3.85 (s, 3H), 3.67 - 3.51 (m, 8H), 3.50 - 3.48 (m, 1H), 3.47 (s, 3H), 3.29 - 3.25 (m, 1H), 3.23 - 3.12 (m, 3H), 2.95 (s, 3H), 2.93 - 2.80 (m, 2H), 2.35 - 2.11 (m, 4H), 2.00 - 1.87 (m, 2H), 1.40 (t, J = 7.4 Hz, 3H), 0.72 - 0.36 (m, 1H), 0.32 - 0.10 (m, 2H), -0.04 - -0.37 (m, 2H). MS (ESI) m/z: 735 [M+H]⁺. |
| I-236 | 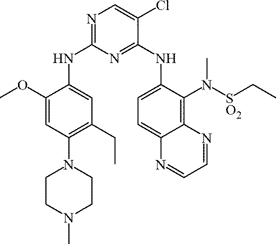 [SMILES: CCc1cc(Nc2ncc(Cl)c(Nc3ccc4nccnc4c3N(C)S(=O)(=O)CC)n2)c(OC)cc1N1CCN(C)CC1] | ¹H NMR (600 MHz, Methanol-d₄) δ 9.00 (d, J = 1.8 Hz, 1H), 8.94 (d, J = 1.8 Hz, 1H), 8.55 - 8.33 (m, 1H), 8.24 - 8.16 (m, 1H), 8.05 (d, J = 9.1 Hz, 1H), 7.34 (s, 1H), 6.81 (s, 1H), 3.83 (s, 3H), 3.46 (s, 3H), 3.21 - 3.15 (m, 2H), 2.95 - 2.92 (m, 5H), 2.88 - 2.78 (m, 2H), 2.53 - 2.29 (m, 2H), 2.23 - 2.13 (m, 2H), 2.01 - 1.86 (m, 2H), 1.40 (t, J = 7.4 Hz, 3H), 1.00 - 0.66 (m, 3H). MS (ESI) m/z: 626 [M+H]⁺. |
| I-237 | 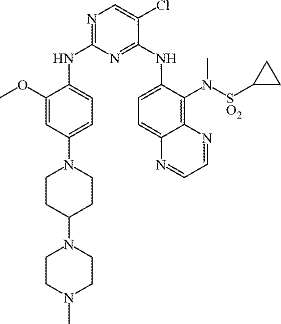 [SMILES: COc1cc(N2CCC(N3CCN(C)CC3)CC2)ccc1Nc1ncc(Cl)c(Nc2ccc3nccnc3c2N(C)S(=O)(=O)C2CC2)n1] | ¹H NMR (600 MHz, Methanol-d₄) δ 8.98 (d, J = 1.9 Hz, 1H), 8.90 (d, J = 1.9 Hz, 1H), 8.70 (d, J = 9.3 Hz, 1H), 8.20 (s, 1H), 8.11 (d, J = 9.3 Hz, 1H), 7.80 (d, J = 8.8 Hz, 1H), 7.01 (s, 1H), 6.82 (d, J = 8.5 Hz, 1H), 3.91 (s, 3H), 3.85 - 3.75 (m, 2H), 3.52 (s, 3H), 3.46 - 3.32 (m, 6H), 3.28 - 3.07 (m, 4H), 3.04 - 2.99 (m, 1H), 2.91 (s, 3H), 2.90 - 2.86 (m, 1H), 2.27 - 2.09 (m, 2H), 2.02 - 1.88 (m, 2H), 1.30 - 1.10 (m, 2H), 0.82 - 0.61 (m, 2H). MS (ESI) m/z: 693 [M+H]⁺. |
| I-238 | 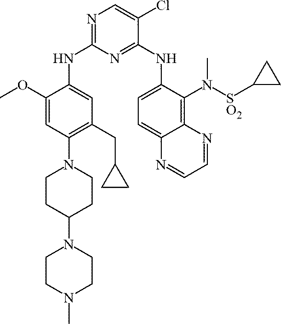 [SMILES: COc1cc(N2CCC(N3CCN(C)CC3)CC2)c(CC2CC2)cc1Nc1ncc(Cl)c(Nc2ccc3nccnc3c2N(C)S(=O)(=O)C2CC2)n1] | ¹H NMR (600 MHz, Methanol-d₄) δ 8.99 (d, J = 1.5 Hz, 1H), 8.90 (s, 1H), 8.59 (s, 1H), 8.18 (s, 1H), 8.02 (d, J = 9.4 Hz, 1H), 7.69 (s, 1H), 6.85 (s, 1H), 3.86 (s, 3H), 3.51 (d, J = 2.9 Hz, 3H), 3.30 - 3.08 (m, 11 H), 2.99 - 2.92 (m, 1H), 2.89 - 2.85 (m, 2H), 2.82 (s, 3H), 2.44 - 2.21 (m, 2H), 2.16 - 2.04 (m, 2H), 1.91 - 1.78 (m, 2H), 1.23 - 1.15 (m, 2H), 0.77 - 0.65 (m, 2H), 0.64 - 0.53 (m, 1H), 0.29 - 0.06 (m, 2H), -0.03 - -0.36 (m, 2H). MS (ESI) m/z: 747 [M+H]⁺. |
| I-239 | 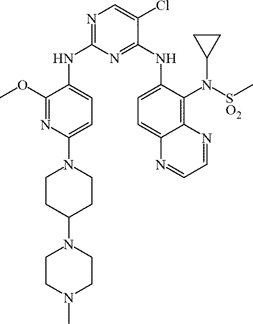 | ¹H NMR (600 MHz, Methanol-d₄) δ 8.96 (d, J = 1.8 Hz, 1H), 8.89 (d, J = 1.9 Hz, 1H), 8.81 - 8.60 (m, 1H), 8.14 (s, 1H), 8.09 - 7.92 (m, 1H), 7.69 - 7.50 (m, 1H), 6.37 (d, J = 8.6 Hz, 1H), 4.60 - 4.45 (m, 2H), 3.88 (s, 3H), 3.73 - 3.67 (m, 1H), 3.59 - 3.45 (m, 8H), 3.35 (s, 3H), 3.35 - 3.32 (m, 1H), 2.98 - 2.94 (m, 2H), 2.93 (s, 3H), 2.25 - 2.10 (m, 2H), 1.82 - 1.64 (m, 2H), 1.26 - 1.13 (m, 1H), 1.07 - 0.92 (m, 1H), 0.51 - 0.37 (m, 1H), 0.35 - 0.21 (m, 1H). MS (ESI) m/z: 694 [M+H]⁺. |
| I-240 | 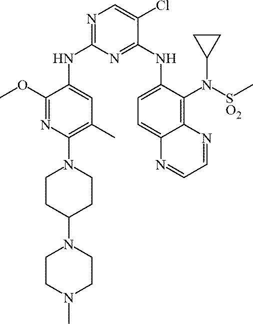 | ¹H NMR (600 MHz, Methanol-d₄) δ 8.96 (d, J = 1.8 Hz, 1H), 8.87 (d, J = 1.9 Hz, 1H), 8.86 - 8.72 (m, 1H), 8.18 (s, 1H), 8.01 - 7.85 (m, 1H), 7.51 (s, 1H), 3.97 (s, 3H), 3.85 - 3.75 (m, 2H), 3.72 - 3.66 (m, 1H), 3.51 - 3.31 (m, 11H), 3.19 - 3.05 (m, 1H), 2.92 - 2.88 (m, 2H), 2.87 (s, 3H), 2.06 (s, 3H), 2.04 - 1.94 (m, 2H), 1.78 - 1.53 (m, 2H), 1.35 - 1.18 (m, 1H), 1.10 - 0.94 (m, 1H), 0.52 - 0.39 (m, 1H), 0.39 - 0.24 (m, 1H). MS (ESI) m/z: 708 [M+H]⁺. |
| I-241 | 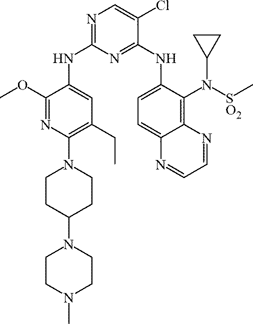 | ¹H NMR (600 MHz, Methanol-d₄) δ 8.99 (d, J = 1.9 Hz, 1H), 8.92 (d, J = 1.9 Hz, 1H), 8.66 - 8.50 (m, 1H), 8.23 (s, 1H), 8.04 (d, J = 9.1 Hz, 1H), 7.84 (s, 1H), 3.95 (s, 3H), 3.77 - 3.66 (m, 1H), 3.62 - 3.41 (m, 10H), 3.32 (s, 3H), 3.29 - 3.26 (m, 1H), 3.12 - 3.01 (m, 2H), 2.94 (s, 3H), 2.55 - 2.33 (m, 2H), 2.28 - 2.08 (m, 2H), 2.02 - 1.88 (m, 2H), 1.20 - 1.08 (m, 1H), 1.02 - 0.92 (m, 1H), 0.90 - 0.70 (m, 3H), 0.49 - 0.35 (m, 1H), 0.31 - 0.22 (m, 1H). MS (ESI) m/z: 722 [M+H]⁺. |
| I-242 | 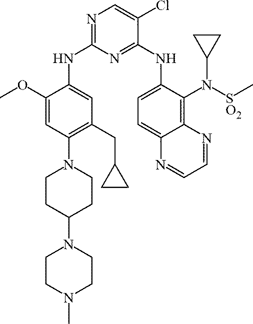 | ¹H NMR (600 MHz, Methanol-d₄) δ 8.92 (d, J = 1.9 Hz, 1H), 8.85 (d, J = 1.9 Hz, 1H), 8.65 - 8.39 (m, 1H), 8.13 (s, 1H), 7.93 (d, J = 9.4 Hz, 1H), 7.61 (s, 1H), 6.81 (s, 1H), 3.79 (s, 3H), 3.67 - 3.61 (m, 1H), 3.53 (s, 8H), 3.26 (s, 3H), 3.24 - 3.22 (m, 1H), 3.18 (t, J = 12.8 Hz, 2H), 2.90 (s, 3H), 2.88 - 2.80 (m, 2H), 2.45 - 2.34 (m, 1H), 2.33 - 2.23 (m, 1H), 2.22 - 2.10 (m, 2H), 1.96 - 1.80 (m, 2H), 1.18 - 1.05 (m, 1H), 1.00 - 0.71 (m, 1H), 0.66 - 0.47 (m, 1H), 0.40 - 0.29 (m, 1H), 0.23 - 0.04 (m, 3H), -0.09 - -0.28 (m, 2H). MS (ESI) m/z: 747 [M+H]⁺. |
| I-243 | 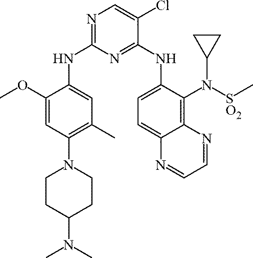 | ¹H NMR (600 MHz, Methanol-d₄) δ 8.99 - 8.91 (m, 1H), 8.87 (d, J = 1.9 Hz, 1H), 8.74 - 8.57 (m, 1H), 8.15 (s, 1H), 7.97 (d, J = 9.3 Hz, 1H), 7.41 (s, 1H), 6.76 (s, 1H), 3.82 (s, 3H), 3.75 - 3.60 (m, 1H), 3.34 (s, 3H), 3.31 - 3.27 (m, 2H), 3.27 - 3.24 (m, 1H), 2.93 (s, 6H), 2.86 - 2.76 (m, 2H), 2.25 - 2.15 (m, 2H), 2.08 (s, 3H), 1.99 - 1.85 (m, 2H), 1.22 - 1.08 (m, 1H), 1.03 - 0.94 (m, 1H), 0.47 - 0.37 (m, 1H), 0.33 - 0.24 (m, 1H). MS (ESI) m/z: 652 [M+H]⁺. |
| I-244 | 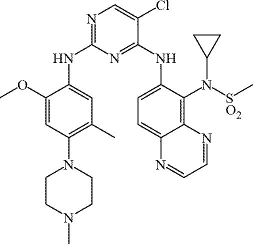 | ¹H NMR (600 MHz, Methanol-d₄) δ 8.94 (d, J = 1.9 Hz, 1H), 8.85 (d, J = 1.8 Hz, 1H), 8.67 (d, J = 9.3 Hz, 1H), 8.13 (s, 1H), 7.95 (d, J = 9.2 Hz, 1H), 7.52 (s, 1H), 6.76 (s, 1H), 3.83 (s, 3H), 3.72 - 3.64 (m, 1H), 3.65 - 3.55 (m, 2H), 3.38 - 3.33 (m, 5H), 3.32 - 3.31 (m, 1H), 3.25 (t, J = 14.4 Hz, 2H), 3.14 - 3.06 (m, 2H), 2.98 (s, 3H), 2.08 (s, 3H), 1.26 - 1.11 (m, 1H), 1.02 - 0.89 (m, 1H), 0.46 - 0.35 (m, 1H), 0.30 - 0.23 (m, 1H). MS (ESI) m/z: 624 [M+H]⁺. |
| I-245 | 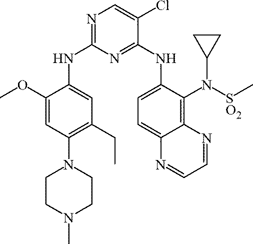 | ¹H NMR (600 MHz, DMSO-d₆) δ 9.00 (d, J = 1.9 Hz, 1H), 8.94 (d, J = 1.9 Hz, 1H), 8.92 - 8.80 (m, 1H), 8.46 (s, 1H), 8.34 (s, 1H), 8.26 (s, 1H), 7.96 (d, J = 9.4 Hz, 1H), 7.49 (s, 1H), 6.81 (s, 1H), 3.80 (s, 3H), 3.63 - 3.52 (m, 3H), 3.38 (s, 3H), 3.26 (d, J = 10.6 Hz, 2H), 3.18 - 3.05 (m, 4H), 2.91 (s, 3H), 2.60 - 2.51 (m, 2H), 1.17 - 1.07 (m, 1H), 1.02 - 0.83 (m, 4H), 0.49 - 0.38 (m, 1H), 0.31 - 0.20 (m, 1H). MS (ESI) m/z: 638 [M+H]⁺. |
| I-246 | 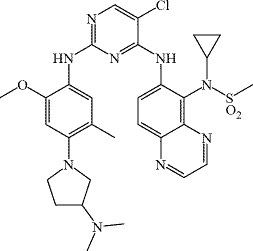 | ¹H NMR (600 MHz, Methanol-d₄) δ 8.97 (d, J = 2.0 Hz, 1H), 8.89 (d, J = 1.9 Hz, 1H), 8.76 - 8.57 (m, 1H), 8.17 (s, 1H), 7.99 (d, J = 9.4 Hz, 1H), 7.37 (d, J = 2.2 Hz, 1H), 6.73 (d, J = 2.1 Hz, 1H), 4.10 - 3.95 (m, 1H), 3.84 (s, 3H), 3.73 - 3.64 (m, 1H), 3.56 - 3.46 (m, 1H), 3.46 - 3.38 (m, 2H), 3.35 (s, 3H), 3.20 - 3.09 (m, 1H), 3.06 - 2.94 (m, 6H), 2.57 - 2.47 (m, 1H), 2.30 - 2.20 (m, 1H), 2.11 (s, 3H), 1.24 - 1.11 (m, 1H), 1.08 - 0.90 (m, 1H), 0.52 - 0.37 (m, 1H), 0.33 - 0.22 (m, 1H). MS (ESI) m/z: 638 [M+H]⁺. |
| I-247 | 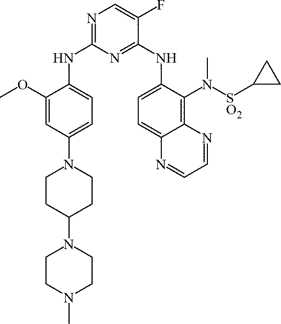 [SMILES: COc1cc(N2CCC(N3CCN(C)CC3)CC2)ccc1Nc1ncc(F)c(Nc2ccc3nccnc3c2N(C)S(=O)(=O)C2CC2)n1] | ¹H NMR (600 MHz, Methanol-d₄) δ 8.99 (d, J = 1.9 Hz, 1H), 8.92 (d, J = 1.9 Hz, 1H), 8.52 (d, J = 9.2 Hz, 1H), 8.13 (d, J = 9.2 Hz, 1H), 8.09 (d, J = 4.3 Hz, 1H), 7.64 (d, J = 8.5 Hz, 1H), 6.94 (d, J = 2.4 Hz, 1H), 6.72 (dd, J = 8.9, 2.4 Hz, 1H), 3.89 (s, 3H), 3.86 - 3.79 (m, 2H), 3.52 (s, 3H), 3.48 - 3.35 (m, 4H), 3.30 - 3.10 (m, 6H), 3.09 - 3.03 (m, 1H), 2.91 (s, 3H), 2.89 - 2.81 (m, 1H), 2.24 - 2.14 (m, 2H), 2.01 - 1.84 (m, 2H), 1.26 - 1.09 (m, 2H), 0.75 - 0.58 (m, 2H). MS (ESI) m/z: 677 [M+H]⁺. |
| I-248 | 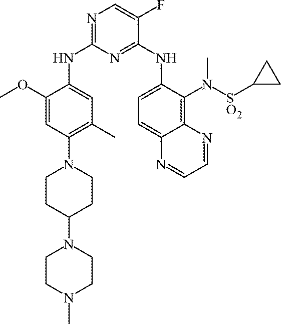 [SMILES: COc1cc(N2CCC(N3CCN(C)CC3)CC2)c(C)cc1Nc1ncc(F)c(Nc2ccc3nccnc3c2N(C)S(=O)(=O)C2CC2)n1] | ¹H NMR (600 MHz, Methanol-d₄) δ 9.00 (d, J = 1.9 Hz, 1H), 8.94 (d, J = 1.9 Hz, 1H), 8.34 (d, J = 9.3 Hz, 1H), 8.15 - 8.05 (m, 2H), 7.29 (s, 1H), 6.78 (s, 1H), 3.84 (s, 3H), 3.63 - 3.40 (m, 11H), 3.27 - 3.18 (m, 3H), 2.94 (s, 3H), 2.89 - 2.69 (m, 3H), 2.26 - 2.12 (m, 2H), 2.02 - 1.84 (m, 5H), 1.23 - 1.04 (m, 2H), 0.74 - 0.56 (m, 2H).MS (ESI) m/z: 691 [M+H]⁺. |
| I-249 | 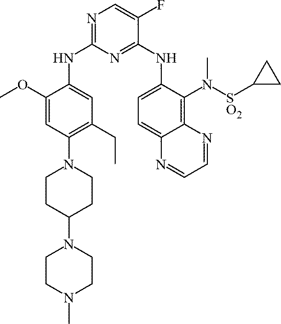 [SMILES: CCc1cc(Nc2ncc(F)c(Nc3ccc4nccnc4c3N(C)S(=O)(=O)C3CC3)n2)c(OC)cc1N1CCC(N2CCN(C)CC2)CC1] | ¹H NMR (600 MHz, Methanol-d₄) δ 9.00 (d, J = 1.9 Hz, 1H), 8.95 (d, J = 1.8 Hz, 1H), 8.28 (d, J = 9.2 Hz, 1H), 8.20 - 8.01 (m, 2H), 7.33 (s, 1H), 6.81 (s, 1H), 3.84 (s, 3H), 3.61 - 3.49 (m, 8H), 3.49 (s, 3H), 3.26 - 3.19 (m, 1H), 3.19 - 3.05 (m, 2H), 2.94 (s, 3H), 2.91 - 2.70 (m, 3H), 2.38 (s, 1H), 2.28 - 2.04 (m, 3H), 2.01 - 1.79 (m, 2H), 1.23 - 1.06 (m, 2H), 0.91 - 0.73 (m, 3H), 0.73 - 0.57 (m, 2H). MS (ESI) m/z: 705 [M+H]⁺. |
| I-250 | 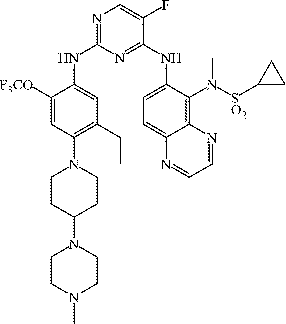 [SMILES: CCc1cc(Nc2ncc(F)c(Nc3ccc4nccnc4c3N(C)S(=O)(=O)C3CC3)n2)c(OC(F)(F)F)cc1N1CCC(N2CCN(C)CC2)CC1] | ¹H NMR (600 MHz, Methanol-d₄) δ 8.98 (d, J = 1.9 Hz, 1H), 8.91 (d, J = 1.9 Hz, 1H), 8.32 (d, J = 9.3 Hz, 1H), 8.14 (d, J = 4.2 Hz, 1H), 7.99 (d, J = 9.2 Hz, 1H), 7.47 (s, 1H), 7.04 (s, 1H), 3.62 - 3.37 (m, 11H), 3.20 - 3.06 (m, 3H), 2.91 (s, 3H), 2.86 - 2.79 (m, 1H), 2.73 (q, J = 11.4 Hz, 2H), 2.53 - 2.42 (m, 1H), 2.41 - 2.26 (m, 1H), 2.23 - 2.11 (m, 2H), 1.93 - 1.81 (m, 2H), 1.22 - 1.10 (m, 2H), 0.87 (t, J = 7.5 Hz, 3H), 0.71 - 0.61 (m, 2H). MS (ESI) m/z: 759 [M+H]⁺. |
| I-251 | 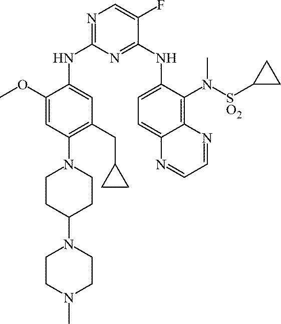 [SMILES: COc1cc(N2CCC(N3CCN(C)CC3)CC2)c(CC2CC2)cc1Nc1ncc(F)c(Nc2ccc3nccnc3c2N(C)S(=O)(=O)C2CC2)n1] | ¹H NMR (600 MHz, Methanol-d₄) δ 9.01 (d, J = 1.8 Hz, 1H), 8.94 (d, J = 1.8 Hz, 1H), 8.34 (d, J = 9.4 Hz, 1H), 8.14 - 8.03 (m, 2H), 7.53 (s, 1H), 6.82 (s, 1H), 3.85 (s, 3H), 3.50 (s, 3H), 3.44 - 3.31 (m, 8H), 3.19 - 3.10 (m, 2H), 3.09 - 2.99 (m, 1H), 2.87 (s, 3H), 2.86 - 2.77 (m, 3H), 2.37 - 2.23 (m, 1H), 2.20 - 2.01 (m, 3H), 1.91 - 1.75 (m, 2H), 1.21 - 1.05 (m, 2H), 0.81 - 0.63 (m, 2H), 0.61 - 0.50 (m, 1H), 0.34 - 0.09 (m, 2H), - 0.07 - -0.24 (m, 2H). MS (ESI) m/z: 731 [M+H]⁺. |
| I-252 | 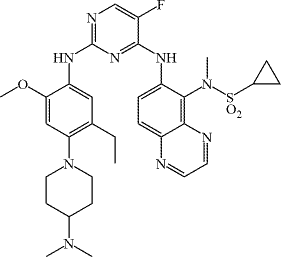 [SMILES: CCc1cc(Nc2ncc(F)c(Nc3ccc4nccnc4c3N(C)S(=O)(=O)C3CC3)n2)c(OC)cc1N1CCC(N(C)C)CC1] | ¹H NMR (600 MHz, Methanol-d₄) δ 9.00 (d, J = 1.7 Hz, 1H), 8.95 (d, J = 1.7 Hz, 1H), 8.31 (d, J = 9.2 Hz, 1H), 8.16 - 8.04 (m, 2H), 7.31 (s, 1H), 6.78 (s, 1H), 3.83 (s, 3H), 3.49 (s, 3H), 3.38 - 3.33 (m, 1H), 3.18 - 3.08 (m, 2H), 2.93 (s, 6H), 2.86 - 2.72 (m, 3H), 2.45 - 2.36 (m, 1H), 2.29 - 2.21 (m, 1H), 2.20 - 2.14 (m, 2H), 1.93 - 1.84 (m, 2H), 1.26 - 1.11 (m, 2H), 0.91 - 0.74 (m, 3H), 0.74 - 0.61 (m, 2H). MS (ESI) m/z: 650 [M+H]⁺. |
| I-253 | 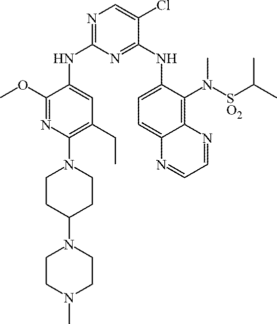 [SMILES: CCc1cc(Nc2ncc(Cl)c(Nc3ccc4nccnc4c3N(C)S(=O)(=O)C(C)C)n2)c(OC)nc1N1CCC(N2CCN(C)CC2)CC1] | ¹H NMR (600 MHz, Methanol-d₄) δ 9.01 (d, J = 1.9 Hz, 1H), 8.94 (d, J = 1.9 Hz, 1H), 8.49 - 8.32 (m, 1H), 8.22 (s, 1H), 8.07 (d, J = 9.2 Hz, 1H), 7.79 (s, 1H), 3.93 (s, 3H), 3.59 - 3.43 (m, 14H), 3.25 - 3.18 (m, 1H), 3.03 - 2.97 (m, 2H), 2.92 (s, 3H), 2.37 - 2.25 (m, 2H), 2.22 - 2.13 (m, 2H), 2.01 - 1.86 (m, 2H), 1.65 (d, J = 6.7 Hz, 3H), 1.28 (d, J = 6.9 Hz, 3H), 0.87 - 0.64 (m, 3H). MS (ESI) m/z: 724 [M+H]⁺. |
| I-254 | 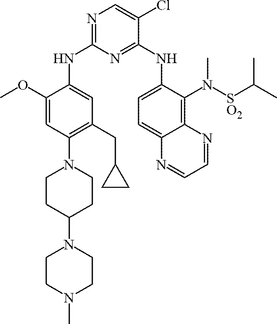 [SMILES: COc1cc(N2CCC(N3CCN(C)CC3)CC2)c(CC2CC2)cc1Nc1ncc(Cl)c(Nc2ccc3nccnc3c2N(C)S(=O)(=O)C(C)C)n1] | ¹H NMR (600 MHz, Methanol-d₄) δ 9.02 (d, J = 1.9 Hz, 1H), 8.96 (d, J = 1.8 Hz, 1H), 8.45 - 8.31 (m, 1H), 8.22 (s, 1H), 8.05 (d, J = 9.3 Hz, 1H), 7.55 (s, 1H), 6.84 (s, 1H), 3.85 (s, 3H), 3.71 - 3.54 (m, 8H), 3.46 (s, 3H), 3.45 - 3.42 (m, 1H), 3.39 - 3.34 (m, 1H), 3.18 (t, J = 13.4 Hz, 2H), 2.98 (s, 3H), 2.88 (t, J = 11.9 Hz, 2H), 2.34 - 2.16 (m, 4H), 2.01 - 1.86 (m, 2H), 1.65 (d, J = 6.7 Hz, 3H), 1.27 (d, J = 6.9 Hz, 3H), 0.69 - 0.40 (m, 1H), 0.31 - 0.07 (m, 2H), -0.07 - -0.39 (m, 2H). MS (ESI) m/z: 749 [M+H]⁺. |
| I-255 | 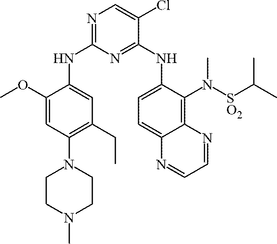 [SMILES: CCc1cc(Nc2ncc(Cl)c(Nc3ccc4nccnc4c3N(C)S(=O)(=O)C(C)C)n2)c(OC)cc1N1CCN(C)CC1] | ¹H NMR (600 MHz, Methanol-d₄) δ 9.01 (d, J = 1.9 Hz, 1H), 8.94 (d, J = 1.9 Hz, 1H), 8.40 (d, J = 9.0 Hz, 1H), 8.21 (s, 1H), 8.05 (d, J = 9.2 Hz, 1H), 7.46 (s, 1H), 6.81 (s, 1H), 3.85 (s, 3H), 3.62 - 3.55 (m, 2H), 3.52 - 3.41 (m, 5H), 3.30 - 3.26 (m, 1H), 3.19 - 3.10 (m, 4H), 2.99 (s, 3H), 2.46 - 2.23 (m, 2H), 1.65 (d, J = 6.6 Hz, 3H), 1.28 (d, J = 6.9 Hz, 3H), 0.85 - 0.62 (m, 3H). MS (ESI) m/z: 640 [M+H]⁺. |
| I-256 | 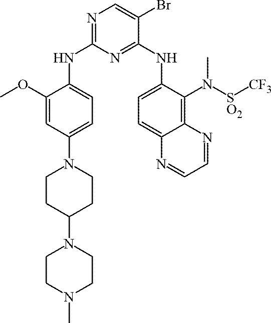 [SMILES: COc1cc(N2CCC(N3CCN(C)CC3)CC2)ccc1Nc1ncc(Br)c(Nc2ccc3nccnc3c2N(C)S(=O)(=O)C(F)(F)F)n1] | ¹H NMR (600 MHz, Methanol-d₄) δ 9.02 (d, J = 1.8 Hz, 1H), 8.99 (d, J = 1.9 Hz, 1H), 8.33 (d, J = 9.2 Hz, 1H), 8.29 (s, 1H), 8.22 (d, J = 9.2 Hz, 1H), 7.53 (d, J = 8.8 Hz, 1H), 6.91 (d, J = 2.5 Hz, 1H), 6.56 (d, J = 8.6 Hz, 1H), 3.85 (s, 3H), 3.79 - 3.71 (m, 2H), 3.59 (s, 3H), 3.52 - 3.39 (m, 4H), 3.30 - 3.14 (m, 6H), 3.13 - 3.07 (m, 1H), 2.93 (s, 3H), 2.25 - 2.15 (m, 2H), 2.00 - 1.89 (m, 2H). MS (ESI) m/z: 765 [M+H]⁺. |
| I-257 | 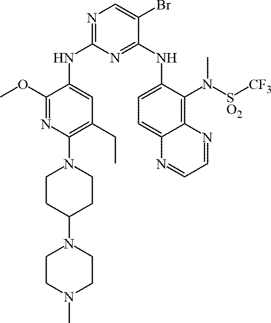 [SMILES: CCc1cc(Nc2ncc(Br)c(Nc3ccc4nccnc4c3N(C)S(=O)(=O)C(F)(F)F)n2)c(OC)nc1N1CCC(N2CCN(C)CC2)CC1] | ¹H NMR (600 MHz, Methanol-d₄) δ 9.03 (d, J = 1.9 Hz, 1H), 8.99 (d, J = 1.9 Hz, 1H), 8.31 (s, 1H), 8.22 (d, J = 9.0 Hz, 1H), 8.15 (d, J = 9.1 Hz, 1H), 7.63 (s, 1H), 3.90 (s, 3H), 3.59 (s, 3H), 3.57 - 3.38 (m, 10H), 3.26 - 3.19 (m, 1H), 2.99 - 2.89 (m, 5H), 2.32 - 2.19 (m, 1H), 2.18 - 2.11 (m, 2H), 2.09 - 1.96 (m, 1H), 1.97 - 1.82 (m, 2H), 0.76 - 0.50 (m, 3H). MS (ESI) m/z: 794 [M+H]⁺. |
| I-258 | 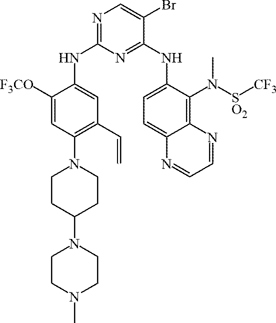 [SMILES: C=Cc1cc(Nc2ncc(Br)c(Nc3ccc4nccnc4c3N(C)S(=O)(=O)C(F)(F)F)n2)c(OC(F)(F)F)cc1N1CCC(N2CCN(C)CC2)CC1] | ¹H NMR (600 MHz, Methanol-d₄) δ 8.99 (d, J = 1.9 Hz, 1H), 8.94 (d, J = 1.9 Hz, 1H), 8.31 (s, 1H), 8.20 (d, J = 9.2 Hz, 1H), 7.97 (d, J = 9.3 Hz, 1H), 7.63 (s, 1H), 6.98 - 6.88 (m, 1H), 6.66 (dd, J = 17.8, 11.0 Hz, 1H), 5.19 - 5.08 (m, 1H), 4.83 - 4.75 (m, 1H), 3.63 - 3.43 (m, 11H), 3.26-3.16 (m, 3H), 2.94 (s, 3H), 2.74 - 2.62 (m, 2H), 2.23 - 2.12 (m, 2H), 1.94 - 1.84 (m, 2H). MS (ESI) m/z: 845 [M+H]⁺. |
| I-259 | 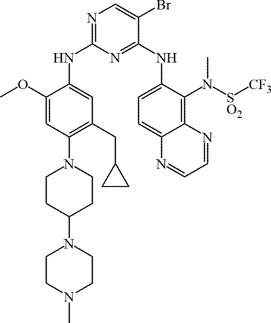 [SMILES: COc1cc(N2CCC(N3CCN(C)CC3)CC2)c(CC2CC2)cc1Nc1ncc(Br)c(Nc2ccc3nccnc3c2N(C)S(=O)(=O)C(F)(F)F)n1] | ¹H NMR (600 MHz, Methanol-d₄) δ 9.03 (d, J = 1.8 Hz, 1H), 9.00 (d, J = 1.8 Hz, 1H), 8.30 (s, 1H), 8.16 (s, 2H), 7.39 (s, 1H), 6.77 (s, 1H), 3.81 (s, 3H), 3.73 - 3.49 (m, 11H), 3.30 - 3.24 (m, 1H), 3.15 - 3.05 (m, 2H), 2.96 (s, 3H), 2.88 - 2.73 (m, 2H), 2.29 - 2.12 (m, 3H), 2.01 - 1.81 (m, 3H), 0.60 - 0.32 (m, 1H), 0.29 - 0.07 (m, 2H), -0.09 - -0.31 (m, 2H). MS (ESI) m/z: 819 [M+H]⁺. |
| I-260 | 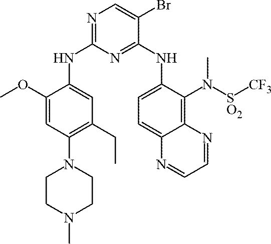 [SMILES: CCc1cc(Nc2ncc(Br)c(Nc3ccc4nccnc4c3N(C)S(=O)(=O)C(F)(F)F)n2)c(OC)cc1N1CCN(C)CC1] | ¹H NMR (600 MHz, Methanol-d₄) δ 9.01 (d, J = 1.8 Hz, 1H), 8.95 (d, J = 1.9 Hz, 1H), 8.38 (d, J = 9.2 Hz, 1H), 8.28 (s, 1H), 8.13 (d, J = 9.2 Hz, 1H), 7.52 (s, 1H), 6.75 (s, 1H), 3.84 (s, 3H), 3.58 (s, 3H), 3.58 - 3.51 (m, 2H), 3.30 - 3.24 (m, 2H), 3.16 - 3.04 (m, 4H), 2.98 (s, 3H), 2.34 - 2.07 (m, 2H), 0.74 - 0.53 (m, 3H). MS (ESI) m/z: 710 [M+H]⁺. |
| I-261 | 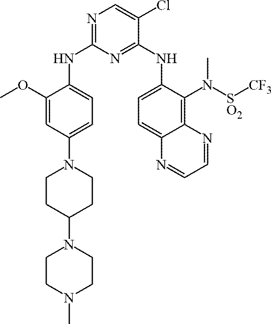 [SMILES: COc1cc(N2CCC(N3CCN(C)CC3)CC2)ccc1Nc1ncc(Cl)c(Nc2ccc3nccnc3c2N(C)S(=O)(=O)C(F)(F)F)n1] | ¹H NMR (600 MHz, Methanol-d₄) δ 9.03 (d, J = 1.8 Hz, 1H), 8.99 (d, J = 1.8 Hz, 1H), 8.28 (d, J = 9.2 Hz, 1H), 8.26 - 8.18 (m, 2H), 7.51 (d, J = 8.8 Hz, 1H), 6.90 (d, J = 2.5 Hz, 1H), 6.60 - 6.51 (m, 1H), 3.85 (s, 3H), 3.81 - 3.72 (m, 2H), 3.59 (s, 3H), 3.53 - 3.42 (m, 4H), 3.32 - 3.23 (m, 4H), 3.19 - 3.10 (m, 3H), 2.94 (s, 3H), 2.26 - 2.17 (m, 2H), 2.00 - 1.89 (m, 2H). MS (ESI) m/z: 721 [M+H]⁺. |
| I-262 | 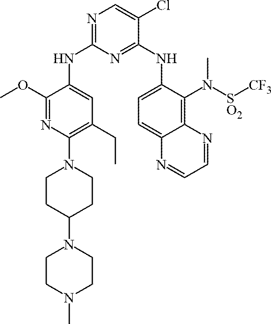 [SMILES: CCc1cc(Nc2ncc(Cl)c(Nc3ccc4nccnc4c3N(C)S(=O)(=O)C(F)(F)F)n2)c(OC)nc1N1CCC(N2CCN(C)CC2)CC1] | ¹H NMR (600 MHz, Methanol-d₄) δ 9.04 (d, J = 1.9 Hz, 1H), 9.00 (d, J = 1.8 Hz, 1H), 8.24 (s, 1H), 8.20 - 8.08 (m, 2H), 7.60 (s, 1H), 3.90 (s, 3H), 3.59 (s, 3H), 3.58 - 3.44 (m, 8H), 3.43 - 3.38 (m, 2H), 3.28 - 3.21 (m, 1H), 2.98 - 2.91 (m, 5H), 2.28 - 2.10 (m, 3H), 2.01 - 1.81 (m, 3H), 0.74 - 0.50 (m, 3H). MS (ESI) m/z: 750 [M+H]⁺. |
| I-263 | 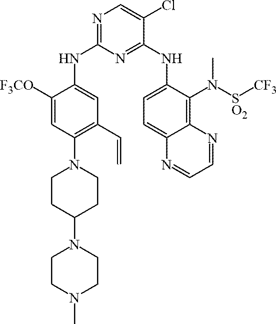 [SMILES: C=Cc1cc(Nc2ncc(Cl)c(Nc3ccc4nccnc4c3N(C)S(=O)(=O)C(F)(F)F)n2)c(OC(F)(F)F)cc1N1CCC(N2CCN(C)CC2)CC1] | ¹H NMR (600 MHz, Methanol-d₄) δ 8.99 (d, J = 1.9 Hz, 1H), 8.94 (d, J = 1.8 Hz, 1H), 8.23 (s, 1H), 8.18 (d, J = 9.3 Hz, 1H), 7.99 (d, J = 9.2 Hz, 1H), 7.63 (s, 1H), 6.92 (s, 1H), 6.64 (dd, J = 17.8, 11.0 Hz, 1H), 5.17 - 5.06 (m, 1H), 4.82 - 4.72 (m, 1H), 3.64 - 3.44 (m, 11H), 3.24 - 3.14 (m, 3H), 2.94 (s, 3H), 2.72 - 2.64 (m, 2H), 2.22 - 2.13 (m, 2H), 1.94 - 1.83 (m, 2H). MS (ESI) m/z: 801 [M+H]⁺. |
| I-264 | 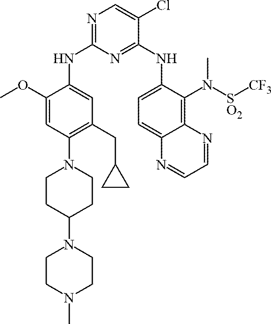 [SMILES: COc1cc(N2CCC(N3CCN(C)CC3)CC2)c(CC2CC2)cc1Nc1ncc(Cl)c(Nc2ccc3nccnc3c2N(C)S(=O)(=O)C(F)(F)F)n1] | ¹H NMR (600 MHz, Methanol-d₄) δ 9.03 (d, J = 1.9 Hz, 1H), 9.01 (d, J = 1.8 Hz, 1H), 8.22 (s, 1H), 8.20 - 8.09 (m, 2H), 7.38 (s, 1H), 6.76 (s, 1H), 3.81 (s, 3H), 3.68 - 3.48 (m, 11H), 3.27 - 3.21 (m, 1H), 3.15 - 3.05 (m, 2H), 2.95 (s, 3H), 2.89 - 2.72 (m, 2H), 2.26 - 2.14 (m, 3H), 1.97 - 1.81 (m, 3H), 0.62 - 0.31 (m, 1H), 0.26 - 0.08 (m, 2H), -0.05 - -0.26 (m, 2H). MS (ESI) m/z: 775 [M+H]⁺. |
| I-265 | 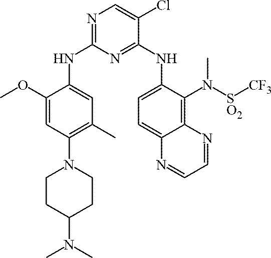 [SMILES: COc1cc(N2CCC(N(C)C)CC2)c(C)cc1Nc1ncc(Cl)c(Nc2ccc3nccnc3c2N(C)S(=O)(=O)C(F)(F)F)n1] | ¹H NMR (600 MHz, Methanol-d₄) δ 9.03 (d, J = 1.9 Hz, 1H), 9.00 (d, J = 1.8 Hz, 1H), 8.22 - 8.12 (m, 3H), 7.14 (s, 1H), 6.66 (s, 1H), 3.80 (s, 3H), 3.56 (s, 3H), 3.30 - 3.26 (m, 1H), 3.17 - 3.07 (m, 2H), 2.93 (s, 6H), 2.79 - 2.67 (m, 2H), 2.20 - 2.12 (m, 2H), 1.92 - 1.83 (m, 2H), 1.67 (brs, 3H). MS (ESI) m/z: 680 [M+H]⁺. |
| I-266 | 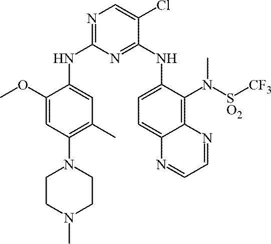 [SMILES: COc1cc(N2CCN(C)CC2)c(C)cc1Nc1ncc(Cl)c(Nc2ccc3nccnc3c2N(C)S(=O)(=O)C(F)(F)F)n1] | ¹H NMR (600 MHz, Methanol-d₄) δ 9.01 (d, J = 1.9 Hz, 1H), 8.95 (d, J = 1.8 Hz, 1H), 8.39 (d, J = 9.2 Hz, 1H), 8.18 (s, 1H), 8.14 (d, J = 9.2 Hz, 1H), 7.44 (s, 1H), 6.70 (s, 1H), 3.83 (s, 3H), 3.64 - 3.52 (m, 5H), 3.30 - 3.24 (m, 2H), 3.21 - 3.13 (m, 2H), 3.03 (t, J = 12.5 Hz, 2H), 2.98 (s, 3H), 1.73 (s, 3H). MS (ESI) m/z: 652 [M+H]⁺. |
| I-267 | 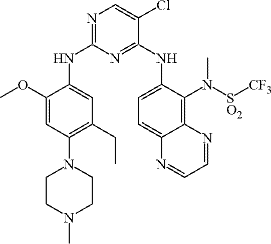 [SMILES: CCc1cc(Nc2ncc(Cl)c(Nc3ccc4nccnc4c3N(C)S(=O)(=O)C(F)(F)F)n2)c(OC)cc1N1CCN(C)CC1] | ¹H NMR (600 MHz, Methanol-d₄) δ 9.04 (d, J = 1.8 Hz, 1H), 9.00 (d, J = 1.8 Hz, 1H), 8.24 (s, 1H), 8.17 (d, J = 9.1 Hz, 1H), 8.13 (d, J = 9.2 Hz, 1H), 7.27 (s, 1H), 6.75 (s, 1H), 3.82 (s, 3H), 3.61 - 3.55 (m, 5H), 3.30 - 3.23 (m, 2H), 3.13 - 3.04 (m, 4H), 2.97 (s, 3H), 2.36 - 1.86 (m, 2H), 0.75 - 0.49 (m, 3H). MS (ESI) m/z: 666 [M+H]⁺. |
| I-268 | 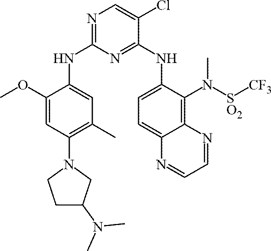 [SMILES: COc1cc(N2CCC(N(C)C)C2)c(C)cc1Nc1ncc(Cl)c(Nc2ccc3nccnc3c2N(C)S(=O)(=O)C(F)(F)F)n1] | ¹H NMR (600 MHz, Methanol-d₄) δ 9.01 (d, J = 1.8 Hz, 1H), 8.96 (d, J = 1.9 Hz, 1H), 8.32 (d, J = 9.2 Hz, 1H), 8.18 (s, 1H), 8.14 (dd, J = 9.3, 2.2 Hz, 1H), 7.27 (d, J = 2.7 Hz, 1H), 6.65 (s, 1H), 4.02 - 3.93 (m, 1H), 3.82 (s, 3H), 3.58 (d, J = 4.1 Hz, 3H), 3.43 - 3.36 (m, 1H), 3.36 - 3.25 (m, 2H), 3.08 - 2.99 (m, 1H), 2.96 (d, J = 6.0 Hz, 6H), 2.52 - 2.38 (m, 1H), 2.25 - 2.11 (m, 1H), 1.86 - 1.68 (m, 3H). MS (ESI) m/z: 666 [M+H]⁺. |
| I-269 | 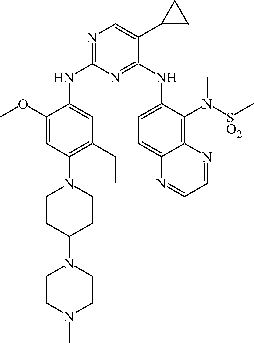 | MS (ESI) m/z: 701 [M+H]⁺. |
| I-270 | 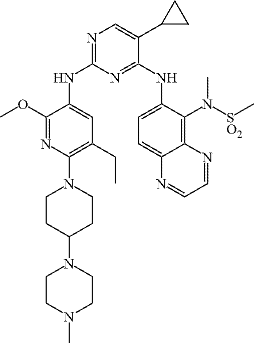 | ¹H NMR (600 MHz, Methanol-d₄) δ 9.00 (d, J = 1.9 Hz, 1H), 8.94 (d, J = 1.9 Hz, 1H), 8.74 - 8.36 (m, 1H), 8.14 - 7.98 (m, 1H), 7.90 - 7.72 (m, 1H), 7.60 (s, 1H), 3.92 (s, 3H), 3.59 - 3.46 (m, 13H), 3.28 - 3.23 (m, 1H), 3.17 (s, 3H), 2.97 - 2.93 (m, 2H), 2.91 (s, 3H), 2.51 - 2.29 (m, 2H), 2.20 - 2.14 (m, 2H), 1.95 - 1.85 (m, 2H), 1.83 - 1.72 (m, 1H), 1.26 - 1.15 (m, 2H), 0.96 - 0.84 (m, 3H), 0.82 - 0.69 (m, 2H). MS (ESI) m/z: 702 [M+H]⁺. |
| I-271 | 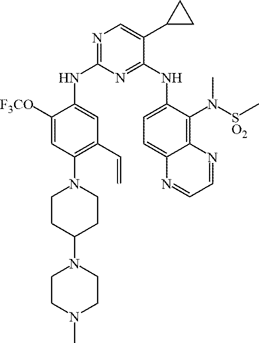 [SMILES: C=Cc1cc(Nc2ncc(C3CC3)c(Nc3ccc4nccnc4c3N(C)S(C)(=O)=O)n2)c(OC(F)(F)F)cc1N1CCC(N2CCN(C)CC2)CC1] | ¹H NMR (600 MHz, Methanol-d₄) δ 8.98 (d, J = 1.9 Hz, 1H), 8.92 (d, J = 1.9 Hz, 1H), 8.56 - 8.35 (m, 1H), 7.95 - 7.80 (m, 2H), 7.69 (s, 1H), 7.06 (s, 1H), 6.94 - 6.81 (m, 1H), 5.53 - 5.38 (m, 1H), 5.17 - 5.03 (m, 1H), 3.48 (s, 3H), 3.45 - 3.31 (m, 10H), 3.16 (s, 3H), 3.09 - 3.01 (m, 1H), 2.90 (d, J = 3.4 Hz, 3H), 2.83 - 2.72 (m, 2H), 2.21 - 2.11 (m, 2H), 1.96 - 1.84 (m, 2H), 1.83 - 1.74 (m, 1H), 1.26 - 1.15 (m, 2H), 0.90 - 0.71 (m, 2H). MS (ESI) m/z: 753 [M+H]⁺. |
| I-272 | 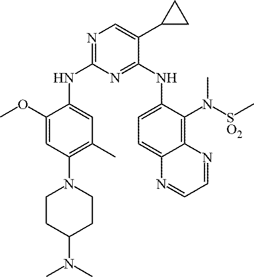 | ¹H NMR (600 MHz, Methanol-d₄) δ 8.99 (d, J = 1.8 Hz, 1H), 8.93 (d, J = 1.9 Hz, 1H), 8.88 - 8.49 (m, 1H), 8.05 (s, 1H), 7.79 (d, J = 35.9 Hz, 1H), 7.28 (s, 1H), 6.81 (s, 1H), 3.84 (s, 3H), 3.50 (s, 3H), 3.43 - 3.32 (m, 2H), 3.30 - 3.23 (m, 1H), 3.19 (s, 3H), 2.95 (s, 6H), 2.87 - 2.80 (m, 2H), 2.26 - 2.19 (m, 2H), 2.20 - 2.04 (m, 3H), 2.00 - 1.88 (m, 2H), 1.80 - 1.72 (m, 1H), 1.31 - 1.22 (m, 1H), 1.21 - 1.13 (m, 1H), 0.94 - 0.79 (m, 1H), 0.77 - 0.67 (m, 1H). MS (ESI) m/z: 632 [M+H]⁺. |
| I-273 | 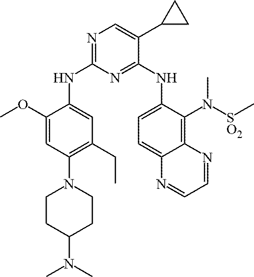 | ¹H NMR (600 MHz, Methanol-d₄) δ 9.00 (d, J = 1.8 Hz, 1H), 8.93 (d, J = 1.8 Hz, 1H), 8.82 - 8.29 (m, 1H), 8.21 - 7.91 (m, 1H), 7.90 - 7.64 (m, 1H), 7.40 - 7.24 (m, 1H), 6.86 (s, 1H), 3.84 (s, 3H), 3.50 (s, 3H), 3.39 - 3.32 (m, 1H), 3.26 - 3.20 (m, 2H), 3.18 (s, 3H), 2.95 (s, 6H), 2.92 - 2.80 (m, 2H), 2.67 - 2.34 (m, 2H), 2.26 - 2.17 (m, 2H), 2.00 - 1.88 (m, 2H), 1.80 - 1.71 (m, 1H), 1.27 - 1.15 (m, 2H), 1.11 - 0.64 (m, 5H). MS (ESI) m/z: 646 [M+H]⁺. |
| I-274 | 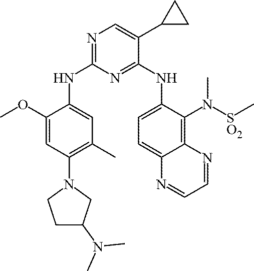 | ¹H NMR (600 MHz, Methanol-d₄) δ 8.99 (d, J = 1.9 Hz, 1H), 8.92 (d, J = 1.9 Hz, 1H), 8.89 - 8.59 (m, 1H), 8.26 - 7.92 (m, 1H), 7.86 - 7.64 (m, 1H), 7.26 (s, 1H), 6.76 (s, 1H), 4.09 - 3.98 (m, 1H), 3.85 (s, 3H), 3.57 - 3.49 (m, 4H), 3.48 - 3.41 (m, 2H), 3.27 - 3.13 (m, 4H), 3.00 (s, 6H), 2.60 - 2.47 (m, 1H), 2.31 - 2.22 (m, 1H), 2.20 - 2.08 (m, 3H), 1.86 - 1.63 (m, 1H), 1.31 - 1.24 (m, 1H), 1.20 - 1.13 (m, 1H), 0.91 - 0.81 (m, 1H), 0.77 - 0.67 (m, 1H). MS (ESI) m/z: 618 [M+H]⁺. |
| I-275 | 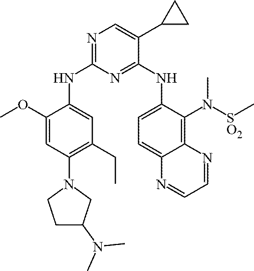 | ¹H NMR (600 MHz, Methanol-d₄) δ 8.99 (d, J = 1.9 Hz, 1H), 8.93 (d, J = 1.9 Hz, 1H), 8.83 - 8.38 (m, 1H), 8.18 - 7.91 (m, 1H), 7.89 - 7.65 (m, 1H), 7.28 (s, 1H), 6.84 (s, 1H), 4.12 - 3.97 (m, 1H), 3.85 (s, 3H), 3.56 - 3.43 (m, 5H), 3.41 - 3.34 (m, 1H), 3.18 (s, 4H), 2.99 (s, 6H), 2.63 - 2.39 (m, 3H), 2.30 - 2.16 (m, 1H), 1.84 - 1.71 (m, 1H), 1.33 - 1.23 (m, 1H), 1.22 - 1.13 (m, 1H), 1.12 - 0.78 (m, 4H), 0.76 - 0.68 (m, 1H). MS (ESI) m/z: 632 [M+H]⁺. |
| I-276 | 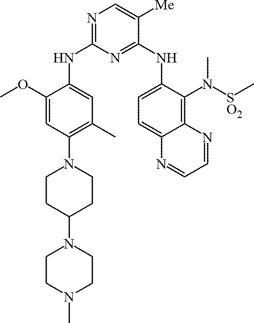 | ¹H NMR (600 MHz, Methanol-d₄) δ 8.99 (d, J = 1.8 Hz, 1H), 8.94 (d, J = 1.9 Hz, 1H), 8.68 - 8.31 (m, 1H), 8.08 (d, J 8.6 Hz, 1H), 7.80 (s, 1H), 7.21 (s, 1H), 6.77 (s, 1H), 3.82 (s, 3H), 3.73 - 3.51 (m, 8H), 3.43 (s, 3H), 3.32 (d, J = 8.9 Hz, 1H), 3.27 - 3.19 (m, 2H), 3.15 (s, 3H), 2.97 (s, 3H), 2.90 - 2.76 (m, 2H), 2.29 (s, 3H), 2.25 - 2.16 (m, 2H), 2.11 - 1.79 (m, 5H). MS (ESI) m/z: 661 [M+H]⁺. |
| I-277 | 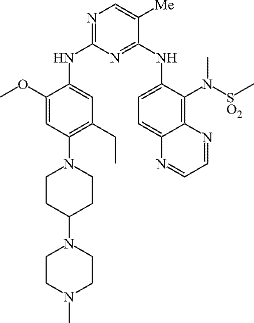 | ¹H NMR (600 MHz, Methanol-d₄) δ 8.99 (d, J = 1.9 Hz, 1H), 8.95 (d, J = 1.9 Hz, 1H), 8.62 - 8.21 (m, 1H), 8.16 - 7.97 (m, 1H), 7.81 (s, 1H), 7.26 (s, 1H), 6.82 (s, 1H), 3.83 (s, 3H), 3.75 - 3.54 (m, 8H), 3.46 - 3.41 (m, 3H), 3.34 - 3.32 (m, 1H), 3.21 - 3.11 (m, 5H), 2.98 (s, 3H), 2.84 (q, J = 11.1, 10.6 Hz, 2H), 2.45 - 2.34 (m, 2H), 2.30 (s, 3H), 2.24 - 2.18 (m, 2H), 2.01 - 1.86 (m, 2H), 1.14 - 0.45 (m, 3H). MS (ESI) m/z: 675 [M+H]⁺. |
| I-278 | 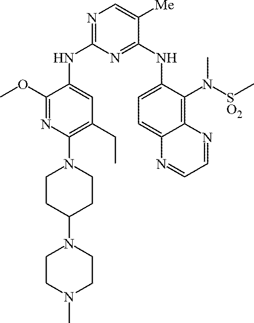 | ¹H NMR (600 MHz, Methanol-d₄) δ 9.00 (d, J = 1.9 Hz, 1H), 8.96 (d, J = 1.9 Hz, 1H), 8.46 - 8.18 (m, 1H), 8.15 - 8.00 (m, 1H), 7.83 (s, 1H), 7.54 (s, 1H), 3.91 (s, 3H), 3.66 - 3.51 (m, 8H), 3.52 - 3.47 (m, 2H), 3.45 (s, 3H), 3.34 - 3.32 (m, 1H), 3.15 (s, 3H), 2.99 - 2.88 (m, 5H), 2.40 - 2.26 (m, 4H), 2.24 - 2.12 (m, 3H), 2.00 - 1.83 (m, 2H), 1.02 - 0.52 (m, 3H). MS (ESI) m/z: 676 [M+H]⁺. |
| I-279 | 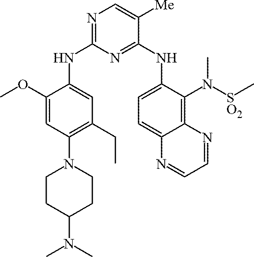 | ¹H NMR (600 MHz, Methanol-d₄) δ 9.00 (d, J = 1.9 Hz, 1H), 8.95 (d, J = 1.9 Hz, 1H), 8.56 - 8.21 (m, 1H), 8.11 - 8.00 (m, 1H), 7.81 (s, 1H), 7.25 (s, 1H), 6.81 (s, 1H), 3.82 (s, 3H), 3.44 (s, 3H), 3.37 - 3.33 (m, 1H), 3.19 - 3.09 (m, 5H), 2.94 (s, 6H), 2.86 - 2.77 (m, 2H), 2.52 - 2.32 (m, 2H), 2.30 (s, 3H), 2.22 - 2.16 (m, 2H), 1.99 - 1.85 (m, 2H), 1.19 - 0.45 (m, 3H). MS (ESI) m/z: 620 [M+H]⁺. |
| I-280 | 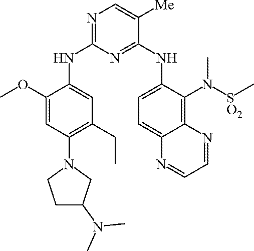 | ¹H NMR (600 MHz, Methanol-d₄) δ 8.99 (d, J = 1.9 Hz, 1H), 8.94 (d, J = 1.8 Hz, 1H), 8.44 (d, J = 44.5 Hz, 1H), 8.05 (s, 1H), 7.81 (s, 1H), 7.23 (s, 1H), 6.94 - 6.66 (m, 1H), 4.13 - 3.96 (m, 1H), 3.83 (s, 3H), 3.51 - 3.40 (m, 5H), 3.34 - 3.31 (m, 1H), 3.21 - 3.10 (m, 4H), 2.98 (s, 6H), 2.61 - 2.39 (m, 2H), 2.35 - 2.15 (m, 5H), 1.24 - 0.42 (m, 3H). MS (ESI) m/z: 606 [M+H]⁺. |
| I-281 | 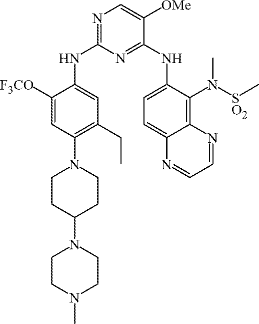 [SMILES: CCc1cc(Nc2ncc(OC)c(Nc3ccc4nccnc4c3N(C)S(C)(=O)=O)n2)c(OC(F)(F)F)cc1N1CCC(N2CCN(C)CC2)CC1] | ¹H NMR (600 MHz, Methanol-d₄) δ 8.99 (d, J = 1.8 Hz, 1H), 8.93 (d, J = 1.9 Hz, 1H), 8.41 (d, J = 9.3 Hz, 1H), 7.97 (d, J = 9.3 Hz, 1H), 7.69 (s, 1H), 7.42 (s, 1H), 7.13 - 7.08 (m, 1H), 4.04 (s, 3H), 3.72 - 3.48 (m, 8H), 3.44 (s, 3H), 3.28 - 3.22 (m, 1H), 3.20 - 3.10 (m, 5H), 2.95 (s, 3H), 2.84 - 2.73 (m, 2H), 2.61 - 2.51 (m, 1H), 2.50 - 2.42 (m, 1H), 2.27 - 2.16 (m, 2H), 2.01 - 1.86 (m, 2H), 0.94 (t, J = 7.6 Hz, 3H). MS (ESI) m/z: 745 [M+H]⁺. |
| I-282 | 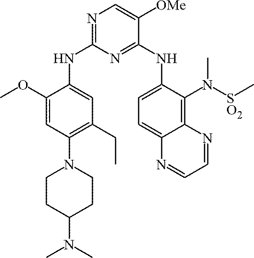 | ¹H NMR (600 MHz, Methanol-d₄) δ 8.99 (d, J = 1.8 Hz, 1H), 8.93 (d, J = 1.8 Hz, 1H), 8.73 - 8.50 (m, 1H), 8.06 (d, J = 9.4 Hz, 1H), 7.62 (s, 1H), 7.30 (s, 1H), 6.85 (s, 1H), 4.02 (s, 3H), 3.84 (s, 3H), 3.46 (s, 3H), 3.35 - 3.32 (m, 1H), 3.25 - 3.15 (m, 5H), 2.94 (s, 6H), 2.91 - 2.81 (m, 2H), 2.59 - 2.39 (m, 2H), 2.29 - 2.15 (m, 2H), 2.00 - 1.86 (m, 2H), 1.05 - 0.80 (m, 3H). MS (ESI) m/z: 636 [M+H]⁺. |
| I-283 | 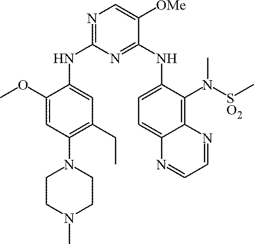 | ¹H NMR (600 MHz, Methanol-d₄) δ 8.99 (d, J = 1.9 Hz, 1H), 8.93 (d, J = 1.9 Hz, 1H), 8.69 - 8.51 (m, 1H), 8.06 (d, J = 9.3 Hz, 1H), 7.65 (s, 1H), 7.37 (s, 1H), 6.88 (s, 1H), 4.02 (s, 3H), 3.86 (s, 3H), 3.65 - 3.56 (m, 2H), 3.47 (s, 3H), 3.35 - 3.31 (m, 2H), 3.24 - 3.12 (m, 7H), 3.00 (s, 3H), 2.59 - 2.35 (m, 2H), 0.96 - 0.81 (m, 3H). MS (ESI) m/z: 608 [M+H]⁺. |
| I-284 | 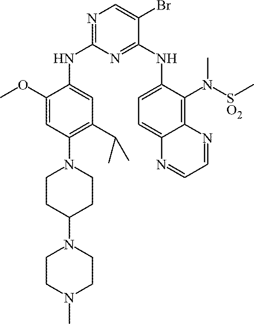 | ¹H NMR (600 MHz, DMSO-d₆) δ 8.98 (d, J = 1.9 Hz, 1H), 8.93 (d, J = 1.8 Hz, 1H), 8.82 - 8.75 (m, 3H), 8.35 - 8.34 (m, 1H), 7.94 - 7.87 (m, 1H), 7.35 - 7.30 (m, 1H), 6.84 (s, 1H), 3.76 (s, 3H), 3.36 (s, 3H), 3.33 (s, 5H), 3.28 - 3.23 (m, 5H), 3.22 (s, 3H), 3.09 - 3.02 (m, 2H), 2.90 (s, 3H), 2.86 - 2.78 (m, 2H), 2.19 - 2.11 (m, 2H), 1.85 - 1.74 (m, 2H), 0.99 - 0.87 (m, 6H). MS (ESI) m/z: 753 [M+H]⁺. |
| I-285 | 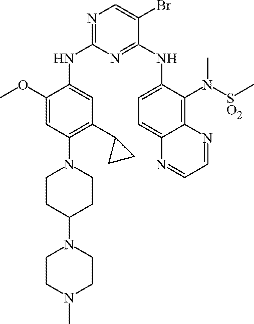 | ¹H NMR (600 MHz, DMSO-d₆) δ 8.98 (d, J = 1.9 Hz, 1H), 8.93 (d, J = 1.8 Hz, 1H), 8.82 - 8.70 (m, 3H), 8.34 (s, 1H), 7.95 - 7.88 (m, 1H), 6.90 (s, 1H), 6.76 (s, 1H), 3.77 (s, 3H), 3.65 (s, 8H), 3.44 - 3.39 (m, 3H), 3.38 (s, 3H), 3.23 (s, 3H), 2.90 (s, 3H), 2.84 - 2.73 (m, 2H), 2.22 - 2.13 (m, 2H), 2.09 - 2.02 (m, 1H), 1.91 - 1.81 (m, 2H), 0.78 - 0.62 (m, 2H), 0.45 - 0.32 (m, 2H). MS (ESI) m/z: 751 [M+H]⁺. |
| I-286 | 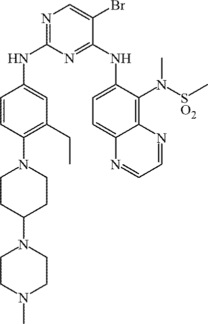 [SMILES: CCc1cc(Nc2ncc(Br)c(Nc3ccc4nccnc4c3N(C)S(C)(=O)=O)n2)ccc1N1CCC(N2CCN(C)CC2)CC1] | ¹H NMR (600 MHz, Methanol-d₄) δ 8.96 - 8.86 (m, 2H), 8.80 (d, J = 1.9 Hz, 1H), 8.20 (s, 1H), 7.94 (d, J = 9.3 Hz, 1H), 7.42 - 7.30 (m, 2H), 7.10 (d, J = 8.6 Hz, 1H), 3.45 (s, 3H), 3.41 - 3.32 (m, 4H), 3.30 - 3.17 (m, 9H), 3.05 - 2.99 (m, 1H), 2.98 - 2.89 (m, 2H), 2.86 (s, 3H), 2.66 (s, 3H), 2.61 (t, J = 8.1 Hz, 2H), 2.19 - 2.06 (m, 2H), 1.92 (q, J = 12.3 Hz, 2H), 1.06 (t, J = 7.6 Hz, 3H). MS (ESI) m/z: 709 [M+H]⁺. |
| I-287 | 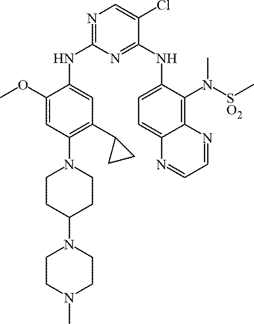 | ¹H NMR (600 MHz, Methanol-d₄) δ 8.94 (d, J = 1.8 Hz, 1H), 8.84 (d, J = 1.9 Hz, 1H), 8.74 - 8.58 (m, 1H), 8.12 (s, 1H), 7.95 (d, J = 9.4 Hz, 1H), 7.14 (s, 1H), 6.76 (s, 1H), 3.83 (s, 3H), 3.47 (s, 3H), 3.46 - 3.41 (m, 2H), 3.31 - 3.04 (m, 11H), 2.96 - 2.90 (m, 1H), 2.85 (t, J = 11.8 Hz, 2H), 2.79 (s, 3H), 2.18 - 2.05 (m, 3H), 1.94 - 1.77 (m, 2H), 0.70 - 0.51 (m, 2H), 0.35 - 0.19 (m, 2H). MS (ESI) m/z: 707 [M+H]⁺. |
| I-288 | 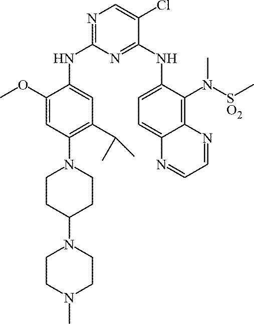 | ¹H NMR (600 MHz, Methanol-d₄) δ 8.94 (d, J = 1.9 Hz, 1H), 8.85 (d, J = 1.8 Hz, 1H), 8.77 - 8.58 (m, 1H), 8.15 (s, 1H), 7.97 (d, J = 9.4 Hz, 1H), 7.54 (s, 1H), 6.84 (s, 1H), 3.82 (s, 3H), 3.45 (s, 3H), 3.31 - 3.15 (m, 11H), 3.15 - 3.06 (m, 3H), 2.97 - 2.90 (m, 1H), 2.90 - 2.83 (m, 2H), 2.79 (s, 3H), 2.15 - 2.03 (m, 2H), 1.89 - 1.77 (m, 2H), 1.00 - 0.82 (m, 6H). MS (ESI) m/z:709 [M+H]⁺. |
| I-289 | 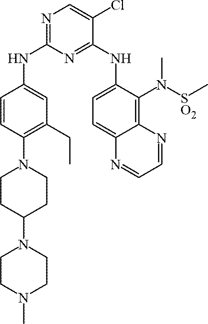 [SMILES: CCc1cc(Nc2ncc(Cl)c(Nc3ccc4nccnc4c3N(C)S(C)(=O)=O)n2)ccc1N1CCC(N2CCN(C)CC2)CC1] | ¹H NMR (600 MHz, Methanol-d₄) δ 8.96 - 8.89 (m, 2H), 8.80 (d, J = 1.8 Hz, 1H), 8.11 (s, 1H), 7.95 (d, J = 9.4 Hz, 1H), 7.42 - 7.28 (m, 2H), 7.09 (d, J = 8.6 Hz, 1H), 3.45 (s, 3H), 3.31 - 3.11 (m, 13H), 3.02 - 2.95 (m, 1H), 2.94 - 2.86 (m, 2H), 2.83 (s, 3H), 2.61 (q, J = 7.6 Hz, 2H), 2.15 - 2.05 (m, 2H), 1.95 - 1.84 (m, 2H), 1.07 (t, J = 7.5 Hz, 3H). MS (ESI) m/z: 665 [M+H]⁺. |
| I-290 | 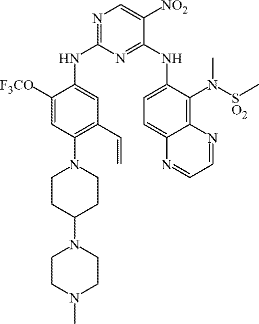 [SMILES: C=Cc1cc(Nc2ncc([N+](=O)[O-])c(Nc3ccc4nccnc4c3N(C)S(C)(=O)=O)n2)c(OC(F)(F)F)cc1N1CCC(N2CCN(C)CC2)CC1] | ¹H NMR (600 MHz, Methanol-d₄) δ 9.17 (s, 1H), 8.93 (s, 1H), 8.84 (s, 1H), 8.45 (s, 1H), 7.71 (s, 1H), 7.65 (s, 1H), 7.02 (s, 1H), 6.85 (s, 1H), 5.31 (d, J = 17.8 Hz, 1H), 4.93 (t, J = 8.7 Hz, 1H), 3.46 (s, 3H), 3.40 - 3.26 (m, 10H), 3.18 (s, 3H), 3.04 - 2.94 (m, 1H), 2.87 (s, 3H), 2.82 - 2.72 (m, 2H), 2.18 - 2.09 (m, 2H), 1.94 - 1.81 (m, 2H). MS (ESI) m/z: 758 [M+H]⁺. |
| I-291 | 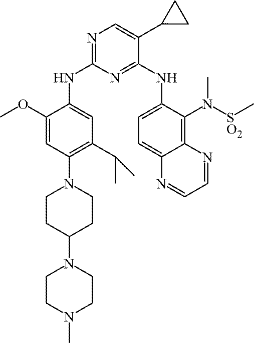 | ¹H NMR (600 MHz, Methanol-d₄) δ 8.96 (d, J = 1.9 Hz, 1H), 8.89 (d, J = 1.9 Hz, 1H), 8.75 - 8.36 (m, 1H), 8.15 - 7.84 (m, 1H), 7.78 - 7.62 (m, 1H), 7.22 (s, 1H), 6.87 (s, 1H), 3.80 (s, 3H), 3.48 (s, 3H), 3.44 - 3.29 (m, 8H), 3.21 - 3.09 (m, 6H), 3.08 - 2.99 (m, 1H), 2.89 - 2.81 (m, 5H), 2.21 - 2.07 (m, 2H), 1.91 - 1.81 (m, 2H), 1.76 - 1.69 (m, 1H), 1.25 - 1.20 (m, 1H), 1.17 - 1.12 (m, 1H), 1.11 - 0.85 (m, 6H), 0.85 - 0.80 (m, 1H), 0.74 - 0.68 (m, 1H). MS (ESI) m/z: 715 [M+H]⁺. |
| I-292 | 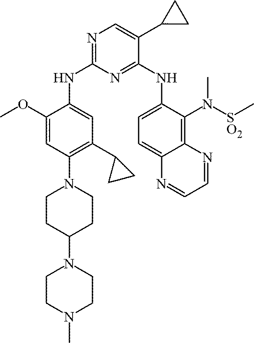 | ¹H NMR (600 MHz, Methanol-d₄) δ 8.98 (d, J = 2.0 Hz, 1H), 8.91 (d, J = 2.0 Hz, 1H), 8.81 - 8.48 (m, 1H), 8.11 - 7.93 (m, 1H), 7.84 - 7.64 (m, 1H), 6.84 (s, 1H), 6.79 (s, 1H), 3.82 (s, 3H), 3.52 - 3.46 (m, 5H), 3.31 - 3.11 (m, 11H), 3.00 - 2.89 (m, 1H), 2.84 - 2.78 (m, 5H), 2.19 - 2.06 (m, 3H), 1.92 - 1.82 (m, 2H), 1.80 - 1.67 (m, 1H), 1.27 - 1.21 (m, 1H), 1.19 - 1.10 (m, 1H), 0.87 - 0.81 (m, 1H), 0.78 - 0.60 (m, 3H), 0.55 - 0.15 (m, 2H). MS (ESI) m/z: 713 [M+H]⁺. |
| I-293 | 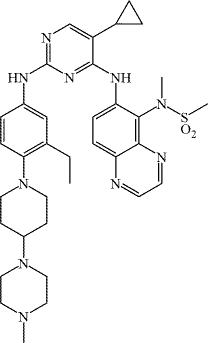 [SMILES: CCc1cc(Nc2ncc(C3CC3)c(Nc3ccc4nccnc4c3N(C)S(C)(=O)=O)n2)ccc1N1CCC(N2CCN(C)CC2)CC1] | ¹H NMR (600 MHz, Methanol-d₄) δ 8.98 (d, J = 1.9 Hz, 1H), 8.92 (d, J = 1.8 Hz, 1H), 8.79 - 8.64 (m, 1H), 8.00 (d, J = 9.0 Hz, 1H), 7.78 (s, 1H), 7.27 (d, J = 2.6 Hz, 1H), 7.21 (dd, J = 8.4, 2.6 Hz, 1H), 7.12 (d, J = 8.5 Hz, 1H), 3.48 (s, 3H), 3.34 - 3.21 (m, 8H), 3.18 (s, 3H), 3.17 - 3.13 (m, 2H), 3.00 - 2.93 (m, 1H), 2.82 (s, 3H), 2.81 - 2.76 (m, 2H), 2.63 - 2.53 (m, 2H), 2.18 - 2.07 (m, 2H), 1.89 - 1.82 (m, 2H), 1.78 - 1.65 (m, 1H), 1.29 - 1.21 (m, 1H), 1.19 - 1.09 (m, 1H), 1.07 - 0.96 (m, 3H), 0.91 - 0.82 (m, 1H), 0.74 - 0.64 (m, 1H). MS (ESI) m/z: 671 [M+H]⁺. |
| I-294 | 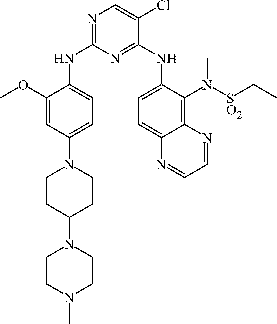 [SMILES: CCS(=O)(=O)N(C)c1c(Nc2nc(Nc3ccc(N4CCC(N5CCN(C)CC5)CC4)cc3OC)ncc2Cl)ccc2nccnc12] | ¹H NMR (600 MHz, Methanol-d₄) δ 8.97 (d, J = 1.9 Hz, 1H), 8.89 (d, J = 1.9 Hz, 1H), 8.65 (d, J = 9.3 Hz, 1H), 8.18 (s, 1H), 8.08 (d, J = 9.3 Hz, 1H), 7.76 (d, J = 8.7 Hz, 1H), 6.80 (dd, J = 8.9, 2.5 Hz, 1H), 3.90 (s, 3H), 3.86 - 3.79 (m, 2H), 3.54 - 3.34 (m, 9H), 3.30 - 3.16 (m, 6H), 3.14 - 3.05 (m, 1H), 2.93 (s, 3H), 2.26 - 2.16 (m, 2H), 2.04 - 1.92 (m, 2H), 1.43 (t, J = 7.4 Hz, 3H). MS (ESI) m/z: 681 [M+H]⁺. |
| I-295 | 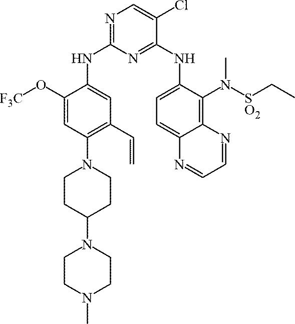 [SMILES: C=Cc1cc(Nc2ncc(Cl)c(Nc3ccc4nccnc4c3N(C)S(=O)(=O)CC)n2)c(OC(F)(F)F)cc1N1CCC(N2CCN(C)CC2)CC1] | ¹H NMR (600 MHz, Methanol-d₄) δ 8.94 (d, J = 1.9 Hz, 1H), 8.83 (d, J = 1.9 Hz, 1H), 8.67 (d, J = 9.5 Hz, 1H), 8.17 (s, 1H), 7.90 - 7.81 (m, 2H), 7.00 (s, 1H), 6.91 (dd, J = 17.7, 11.0 Hz, 1H), 5.36 (d, J = 17.7 Hz, 1H), 4.98 (d, J = 11.1 Hz, 1H), 3.53 - 3.47 (m, 1H), 3.45 (s, 3H), 3.43 - 3.37 (m, 1H), 3.29 -2.96 (m, 10H), 2.87 -2.79 (m, 4H), 2.76 - 2.68 (m, 2H), 2.13 - 1.99 (m, 2H), 1.88 - 1.74 (m, 2H), 1.42 (t, J = 7.4 Hz, 3H). MS (ESI) m/z: 761 [M+H]⁺. |
| I-296 | 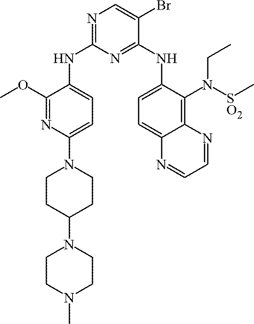 | ¹H NMR (600 MHz, Methanol-d₄) δ 8.89 (d, J = 1.9 Hz, 1H), 8.87 - 8.78 (m, 2H), 8.15 (s, 1H), 7.97 - 7.90 (m, 1H), 7.68 (d, J = 4.9 Hz, 1H), 6.29 (d, J = 8.4 Hz, 1H), 4.45 - 4.37 (m, 2H), 4.17 - 4.07 (m, 1H), 3.85 (s, 3H), 3.82 - 3.74 (m, 1H), 3.29 - 3.02 (m, 11H), 2.98 (d, J = 14.1 Hz, 1H), 2.91 - 2.85 (m, 2H), 2.80 (s, 3H), 2.08 - 1.97 (m, 2H), 1.73 - 1.58 (m, 2H), 1.03 (t, J = 7.1 Hz, 3H). MS (ESI) m/z: 726 [M+H]⁺. |
| I-297 | 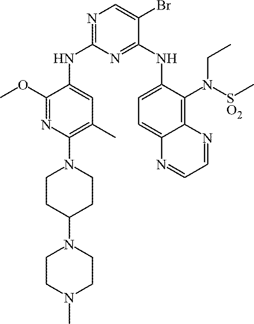 | ¹H NMR (600 MHz, DMSO-d₆) δ 8.95 (d, J = 1.8 Hz, 1H), 8.91 (d, J = 1.8 Hz, 1H), 8.88 - 8.76 (m, 1H), 8.74 - 8.69 (m, 1H), 8.62 (s, 1H), 8.33 (s, 1H), 7.86 - 7.59 (m, 1H), 7.42 (s, 1H), 4.11 - 4.05 (m, 1H), 3.93 (s, 3H), 3.89 - 3.81 (m, 2H), 3.72 - 3.66 (m, 1H), 3.38 (s, 3H), 3.22 (s, 3H), 3.14 - 2.54 (m, 11H), 2.04 (s, 3H), 1.84 - 1.60 (m, 2H), 1.42 - 1.20 (m, 2H), 1.00 (t, J = 7.1 Hz, 3H). MS (ESI) m/z: 740 [M+H]⁺. |
| I-298 | 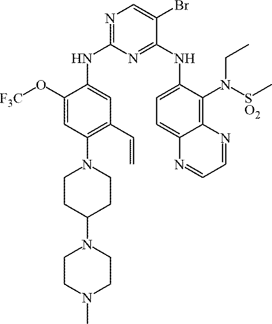 [SMILES: C=Cc1cc(Nc2ncc(Br)c(Nc3ccc4nccnc4c3N(CC)S(C)(=O)=O)n2)c(OC(F)(F)F)cc1N1CCC(N2CCN(C)CC2)CC1] | ¹H NMR (600 MHz, Methanol-d₄) δ 8.92 (d, J = 1.9 Hz, 1H), 8.83 (d, J = 1.9 Hz, 1H), 8.64 (d, J = 9.3 Hz, 1H), 8.26 (s, 1H), 7.85 - 7.77 (m, 2H), 7.03 - 6.99 (m, 1H), 6.93 (dd, J = 17.8, 11.1 Hz, 1H), 5.38 (d, J = 17.7 Hz, 1H), 5.01 (d, J = 11.0 Hz, 1H), 4.14 (dq, J = 14.5, 7.3 Hz, 1H), 3.79 (dq, J = 14.1, 7.1 Hz, 1H), 3.41 - 3.31 (m, 8H), 3.16 (s, 3H), 2.99 (d, J = 11.4 Hz, 2H), 2.89 - 2.84 (m, 4H), 2.79 - 2.72 (m, 2H), 2.17 - 2.08 (m, 2H), 1.92 - 1.82 (m, 2H), 1.03 (t, J = 7.2 Hz, 3H). MS (ESI) m/z: 805 [M+H]⁺. |
| I-299 | 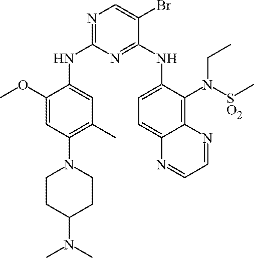 | ¹H NMR (600 MHz, Methanol-d₄) δ 8.91 (d, J = 1.9 Hz, 1H), 8.85 - 8.78 (m, 2H), 8.18 (s, 1H), 7.96 (d, J = 9.3 Hz, 1H), 7.62 (s, 1H), 6.71 (s, 1H), 4.16 - 4.07 (m, 1H), 3.82 (s, 3H), 3.81 - 3.76 (m, 1H), 3.34 - 3.28 (m, 1H), 3.26 - 3.19 (m, 2H), 3.17 (s, 3H), 2.93 (s, 6H), 2.82 - 2.74 (m, 2H), 2.23 - 2.13 (m, 2H), 2.06 (s, 3H), 1.91 (qdd, J = 11.5, 6.2, 4.1 Hz, 2H), 1.02 (t, J= 7.2 Hz, 3H). MS (ESI) m/z: 684 [M+H]⁺. |
| I-300 | 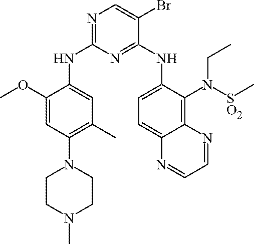 | ¹H NMR (600 MHz, Methanol-d₄) δ 8.93 (d, J = 1.8 Hz, 1H), 8.85 (d, J = 1.9 Hz, 1H), 8.80 (d, J = 9.3 Hz, 1H), 8.22 (s, 1H), 8.00 (d, J = 9.3 Hz, 1H), 7.67 (s, 1H), 6.76 (s, 1H), 4.19 - 4.07 (m, 1H), 3.85 (s, 3H), 3.84 - 3.77 (m, 1H), 3.66 - 3.53 (m, 2H), 3.36 - 3.31 (m, 2H), 3.29 - 3.22 (m, 2H), 3.18 (s, 3H), 3.14 - 3.04 (m, 2H), 3.00 (s, 3H), 2.08 (s, 3H), 1.02 (t, J = 7.2 Hz, 3H). MS (ESI) m/z: 656 [M+H]⁺. |
| I-301 | 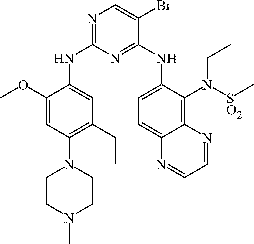 | ¹H NMR (600 MHz, DMSO-d₆) δ 8.98 (d, J = 1.9 Hz, 1H), 8.94 (d, J = 1.9 Hz, 1H), 8.85 (d, J = 21.5 Hz, 1H), 8.60 (s, 1H), 8.42 (s, 1H), 8.34 (s, 1H), 7.97 (d, J = 9.3 Hz, 1H), 7.49 (s, 1H), 6.80 (s, 1H), 4.06 (dq, J = 14.5, 7.3 Hz, 1H), 3.80 (s, 3H), 3.69 (dq, J = 13.9, 7.0 Hz, 1H), 3.57 - 3.48 (m, 2H), 3.27 - 3.19 (m, 5H), 3.17 - 3.10 (m, 2H), 3.09 - 2.97 (m, 2H), 2.91 (s, 3H), 2.59 - 2.53 (m, 1H), 2.48 - 2.38 (m, 1H), 0.98 (t, J = 7.2 Hz, 3H), 0.91 - 0.79 (m, 3H). MS (ESI) m/z: 670 [M+H]⁺. |
| I-302 | 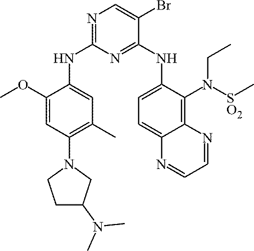 | ¹H NMR (600 MHz, Methanol-d₄) δ 8.91 (d, J = 1.5 Hz, 1H), 8.85 - 8.78 (m, 2H), 8.19 (s, 1H), 7.97 (d, J = 9.4 Hz, 1H), 7.58 (s, 1H), 6.72 (s, 1H), 4.19 - 4.07 (m, 1H), 4.05 - 3.97 (m, 1H), 3.83 (s, 3H), 3.82 - 3.76 (m, 1H), 3.49 - 3.40 (m, 1H), 3.41 - 3.33 (m, 2H), 3.17 (s, 3H), 3.14 - 3.06 (m, 1H), 2.98 (d, J = 5.5 Hz, 6H), 2.53 - 2.43 (m, 1H), 2.20 (dq, J = 14.2, 7.5 Hz, 1H), 2.09 (s, 3H), 1.03 (t, J = 7.1 Hz, 3H). MS (ESI) m/z: 670 [M+H]⁺. |
| I-303 | 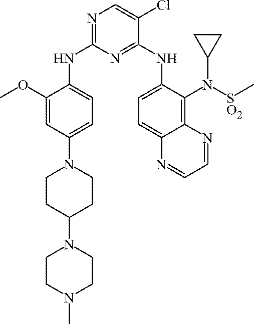 | ¹H NMR (600 MHz, Methanol-d₄) δ 8.93 (d, J = 1.8 Hz, 1H), 8.85 (d, J = 9.3 Hz, 1H), 8.81 (d, J = 1.8 Hz, 1H), 8.13 (s, 1H), 7.98 (d, J = 9.3 Hz, 1H), 7.94 - 7.85 (m, 1H), 7.00 (s, 1H), 3.90 (s, 3H), 3.82 - 3.75 (m, 2H), 3.70 - 3.65 (m, 1H), 3.38 (s, 3H), 3.31 - 3.19 (m, 5H), 3.19 - 2.96 (m, 4H), 2.95 - 2.88 (m, 2H), 2.87 (s, 3H), 2.21 - 2.12 (m, 2H), 1.99 - 1.90 (m, 2H), 1.24 - 1.17 (m, 1H), 1.05 - 0.93 (m, 1H), 0.49 - 0.37 (m, 1H), 0.34 - 0.22 (m, 1H). MS (ESI) m/z: 693 [M+H]⁺. |
| I-304 | 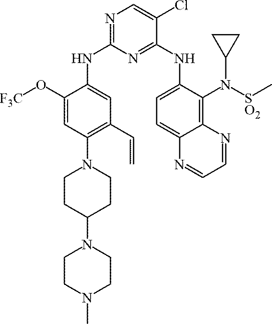 [SMILES: C=Cc1cc(Nc2ncc(Cl)c(Nc3ccc4nccnc4c3N(C3CC3)S(C)(=O)=O)n2)c(OC(F)(F)F)cc1N1CCC(N2CCN(C)CC2)CC1] | ¹H NMR (600 MHz, Methanol-d₄) δ 8.93 (d, J = 1.9 Hz, 1H), 8.82 (d, J = 1.8 Hz, 1H), 8.73 (d, J = 9.7 Hz, 1H), 8.16 (s, 1H), 7.91 - 7.72 (m, 2H), 7.01 (s, 1H), 6.95 (dd, J = 17.8, 11.0 Hz, 1H), 5.39 (d, J = 17.7 Hz, 1H), 5.01 (d, J = 11.0 Hz, 1H), 3.68 - 3.63 (m, 1H), 3.34 (s, 3H), 3.28 - 2.99 (m, 10H), 2.83 - 2.78 (m, 4H), 2.76 - 2.71 (m, 2H), 2.11 - 2.01 (m, 2H), 1.87 - 1.79 (m, 2H), 1.25 - 1.20 (m, 1H), 1.03 - 0.97 (m, 1H), 0.45 - 0.38 (m, 1H), 0.33 - 0.25 (m, 1H). MS (ESI) m/z: 773 [M+H]⁺. |
| I-305 | 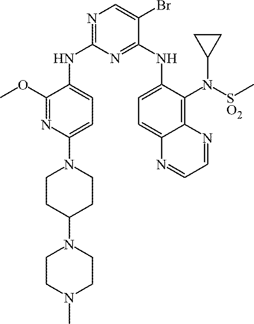 | ¹H NMR (600 MHz, Methanol-d₄) δ 8.90 (d, J = 1.9 Hz, 1H), 8.86 - 8.75 (m, 2H), 8.12 (s, 1H), 7.94 - 7.81 (m, 1H), 7.70 - 7.61 (m, 1H), 6.28 (d, J = 8.5 Hz, 1H), 4.48 - 4.38 (m, 2H), 3.84 (s, 3H), 3.68 - 3.62 (m, 1H), 3.37 (s, 3H), 3.36 - 3.34 (m, 1H), 3.31 - 3.14 (m, 7H), 3.07 - 3.01 (m, 1H), 2.89 - 2.84 (m, 2H), 2.83 (s, 3H), 2.10 - 2.02 (m, 2H), 1.73 - 1.61 (m, 2H), 1.33 - 1.27 (m, 1H), 1.06 - 0.95 (m, 1H), 0.48 - 0.35 (m, 1H), 0.35 - 0.25 (m, 1H). MS (ESI) m/z: 738 [M+H]⁺. |
| I-306 | 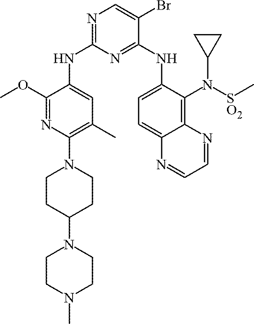 | ¹H NMR (600 MHz, DMSO-d₆) δ 8.98 (d, J = 1.9 Hz, 1H), 8.91 (d, J = 1.8 Hz, 1H), 8.87 - 8.81 (m, 1H), 8.77 (s, 1H), 8.32 (d, J = 12.6 Hz, 2H), 7.75 (s, 1H), 7.43 (s, 1H), 4.10 - 3.85 (m, 7H), 3.71 - 3.59 (m, 4H), 3.40 (s, 3H), 3.08 - 2.81 (m, 4H), 2.77 - 2.70 (m, 5H), 2.05 (s, 3H), 1.95 - 1.80 (m, 2H), 1.56 - 1.37 (m, 2H), 1.31 - 1.20 (m, 1H), 1.07 - 0.96 (m, 1H), 0.50 - 0.37 (m, 1H), 0.30 - 0.23 (m, 1H). MS (ESI) m/z: 752 [M+H]⁺. |
| I-307 | 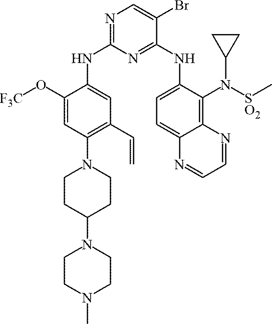 [SMILES: C=Cc1cc(Nc2ncc(Br)c(Nc3ccc4nccnc4c3N(C3CC3)S(C)(=O)=O)n2)c(OC(F)(F)F)cc1N1CCC(N2CCN(C)CC2)CC1] | MS (ESI) m/z: 817 [M+H]⁺. |
| I-308 | 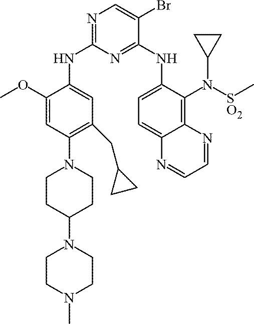 | ¹H NMR (600 MHz, Methanol-d₄) δ 8.93 (d, J = 1.9 Hz, 1H), 8.84 (d, J = 1.9 Hz, 1H), 8.71 - 8.57 (m, 1H), 8.19 (s, 1H), 7.92 (d, J = 9.3 Hz, 1H), 7.76 (s, 1H), 6.81 (s, 1H), 3.82 (s, 3H), 3.73 - 3.62 (m, 1H), 3.38 - 3.22 (m, 11H), 3.18 - 3.09 (m, 2H), 3.03 - 2.95 (m, 1H), 2.88 - 2.76 (m, 5H), 2.49 - 2.42 (m, 1H), 2.33 - 2.21 (m, 1H), 2.15 - 2.05 (m, 2H), 1.90 - 1.75 (m, 2H), 1.30 - 1.16 (m, 1H), 1.04 - 0.91 (m, 1H), 0.64 - 0.47 (m, 1H), 0.41 - 0.31 (m, 1H), 0.32 - 0.23 (m, 1H), 0.20 - 0.01 (m, 2H), -0.12 - -0.30 (m, 2H). MS (ESI) m/z: 791 [M+H]⁺. |
| I-309 | 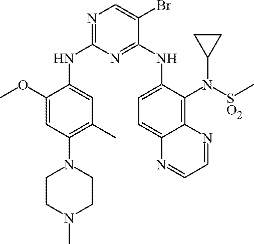 | ¹H NMR (600 MHz, Methanol-d₄) δ 8.98 -8.89 (m, 1H), 8.82 (d, J = 1.8 Hz, 1H), 8.81 - 8.77 (m, 1H), 8.18 (s, 1H), 7.94 (d, J = 9.3 Hz, 1H), 7.67 (s, 1H), 6.75 (s, 1H), 3.84 (s, 3H), 3.72 - 3.64 (m, 1H), 3.63 - 3.53 (m, 2H), 3.36 (s, 3H), 3.35 - 3.31 (m, 2H), 3.29 - 3.19 (m, 2H), 3.09 (t, J = 12.5 Hz, 2H), 2.99 (s, 3H), 2.09 (s, 3H), 1.29 - 1.24 (m, 1H), 1.01 - 0.93 (m, 1H), 0.45 - 0.35 (m, 1H), 0.35 - 0.26 (m, 1H). MS (ESI) m/z: 668 [M+H]⁺. |
| I-310 | 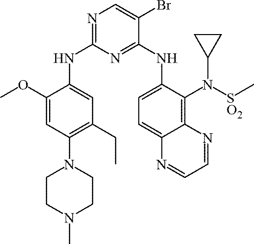 | ¹H NMR (600 MHz, DMSO-d₆) δ 10.14 (s, 1H), 8.99 (d, J = 1.9 Hz, 1H), 8.94 (d, J =1.9 Hz, 1H), 8.90 - 8.76 (m, 1H), 8.48 (s, 1H), 8.32 (d, J = 2.9 Hz, 2H), 7.94 (d, J = 9.4 Hz, 1H), 7.47 (s, 1H), 6.82 (s, 1H), 3.80 (s, 3H), 3.65 - 3.59 (m, 1H), 3.57 - 3.48 (m, 2H), 3.38 (s, 3H), 3.31 - 3.21 (m, 2H), 3.19 - 3.05 (m, 4H), 2.91 (s, 3H), 2.59 - 2.52 (m, 1H), 2.49 - 2.42 (m, 1H), 1.26 - 1.15 (m, 1H), 1.00 - 0.95 (m, 1H), 0.94 - 0.85 (m, 3H), 0.46 - 0.35 (m, 1H), 0.31 - 0.16 (m, 1H). MS (ESI) m/z: 682 [M+H]⁺. |
| I-311 | 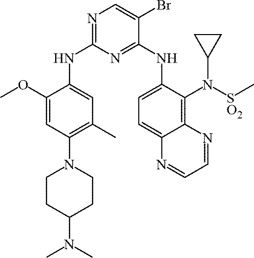 | ¹H NMR (600 MHz, Methanol-d₄) δ 8.91 (d, J = 2.0 Hz, 1H), 8.84 - 8.80 (m, 1H), 8.80 (d, J = 1.9 Hz, 1H), 8.14 (s, 1H), 7.90 (d, J = 9.3 Hz, 1H), 7.61 (s, 1H), 6.70 (s, 1H), 3.80 (s, 3H), 3.69 - 3.63 (m, 1H), 3.36 (s, 3H), 3.31 - 3.26 (m, 1H), 3.25 - 3.13 (m, 2H), 2.92 (s, 6H), 2.76 (t, J = 11.6 Hz, 2H), 2.22 - 2.14 (m, 2H), 2.06 (s, 3H), 1.97 - 1.85 (m, 2H), 1.33 - 1.19 (m, 1H), 1.03 - 0.91 (m, 1H), 0.45 - 0.37 (m, 1H), 0.35 - 0.27 (m, 1H). MS (ESI) m/z: 696 [M+H]⁺. |
| I-312 | 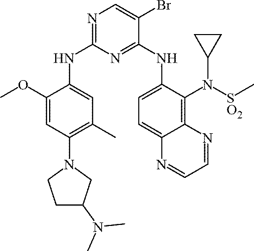 | ¹H NMR (600 MHz, Methanol-d₄) δ 8.92 (d, J = 1.8 Hz, 1H), 8.85 - 8.76 (m, 2H), 8.15 (s, 1H), 7.91 (d, J = 9.3 Hz, 1H), 7.56 - 7.52 (m, 1H), 6.71 (d, J = 2.1 Hz, 1H), 4.04 - 3.97 (m, 1H), 3.82 (d, J = 1.7 Hz, 3H), 3.69 - 3.63 (m, 1H), 3.51 - 3.43 (m, 1H), 3.40 - 3.34 (m, 5H), 3.15 - 3.06 (m, 1H), 2.98 (d, J = 4.2 Hz, 6H), 2.52 - 2.43 (m, 1H), 2.24 - 2.16 (m, 1H), 2.10 (s, 3H), 1.29 - 1.24 (m, 1H), 1.04 - 0.93 (m, 1H), 0.44 - 0.35 (m, 1H), 0.34 - 0.26 (m, 1H). MS (ESI) m/z: 682 [M+H]⁺. |
| I-313 | 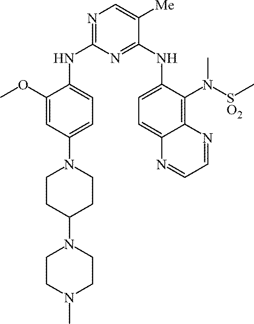 | ¹H NMR (600 MHz, Methanol-d₄) δ 8.98 (d, J = 1.8 Hz, 1H), 8.93 (d, J = 2.0 Hz, 1H), 8.61 - 8.47 (m, 1H), 8.09 (d, J = 9.3 Hz, 1H), 7.77 (s, 1H), 7.31 - 7.23 (m, 1H), 6.77 (s, 1H), 3.85 - 3.81 (m, 5H), 3.46 (s, 3H), 3.42 - 3.33 (m, 4H), 3.31 - 3.19 (m, 4H), 3.16 (s, 3H), 3.07 - 3.00 (m, 1H), 2.97 - 2.89 (m, 2H), 2.87 (s, 3H), 2.28 (s, 3H), 2.16 - 2.11 (m, 2H), 1.87 - 1.78 (m, 2H). MS (ESI) m/z: 647 [M+H]⁺. |
| I-314 | 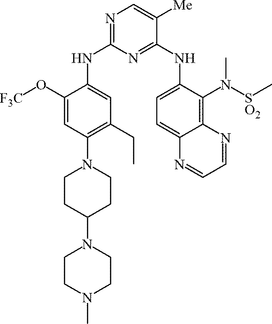 [SMILES: CCc1cc(Nc2ncc(C)c(Nc3ccc4nccnc4c3N(C)S(C)(=O)=O)n2)c(OC(F)(F)F)cc1N1CCC(N2CCN(C)CC2)CC1] | ¹H NMR (600 MHz, Methanol-d₄) δ 8.98 (d, J = 1.9 Hz, 1H), 8.94 (d, J = 1.9 Hz, 1H), 8.24 (d, J = 8.7 Hz, 1H), 7.96 (d, J = 9.2 Hz, 1H), 7.87 (s, 1H), 7.35 (s, 1H), 7.04 (s, 1H), 3.41 (s, 3H), 3.38 - 3.20 (m, 8H), 3.14 (s, 3H), 3.11 - 3.04 (m, 2H), 3.00 - 2.93 (m, 1H), 2.85 (s, 3H), 2.75 -2.68 (m, 2H), 2.51 - 2.42 (m, 1H), 2.36 -2.32 (m, 1H), 2.31 (s, 3H), 2.15 - 2.07 (m, 2H), 1.87 - 1.76 (m, 2H), 0.92 - 0.83 (m, 3H). MS (ESI) m/z: 729 [M+H]⁺. |
| I-315 | 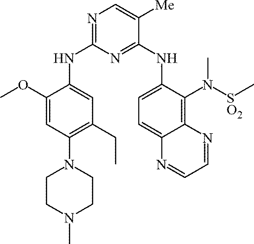 | ¹H NMR (600 MHz, Methanol-d₄) δ 8.99 (d, J = 1.9 Hz, 1H), 8.94 (d, J = 1.9 Hz, 1H), 8.42 (d, J = 9.1 Hz, 1H), 8.05 (d, J = 9.2 Hz, 1H), 7.83 (s, 1H), 7.37 (s, 1H), 6.83 (s, 1H), 3.85 (s, 3H), 3.66 - 3.51 (m, 2H), 3.44 (s, 3H), 3.32 - 3.25 (m, 2H), 3.18 - 3.11 (m, 7H), 2.99 (s, 3H), 2.44 - 2.36 (m, 1H), 2.34 - 2.24 (m, 4H), 0.85 - 0.70 (m, 3H). MS (ESI) m/z: 592 [M+H]⁺. |
| I-316 | 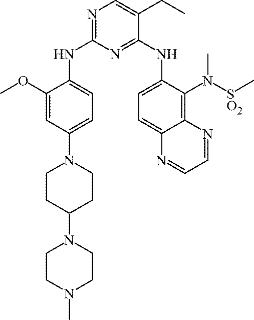 | ¹H NMR (600 MHz, Methanol-d₄) δ 8.99 (d, J = 1.9 Hz, 1H), 8.94 (d, J = 1.9 Hz, 1H), 8.59 - 8.49 (m, 1H), 8.11 (d, J = 9.3 Hz, 1H), 7.73 (s, 1H), 7.32 - 7.21 (m, 1H), 6.77 (s, 1H), 6.57 - 6.47 (m, 1H), 3.89 - 3.79 (m, 5H), 3.46 (s, 3H), 3.42 - 3.32 (m, 4H), 3.30 - 3.17 (m, 4H), 3.16 (s, 3H), 3.04 - 2.97 (m, 1H), 2.92 (t, J = 12.2 Hz, 2H), 2.87 (s, 3H), 2.73 - 2.66 (m, 2H), 2.16 - 2.10 (m, 2H), 1.87 - 1.78 (m, 2H), 1.37 (t, J = 7.5 Hz, 3H). MS (ESI) m/z: 661 [M+H]⁺. |
| I-317 | 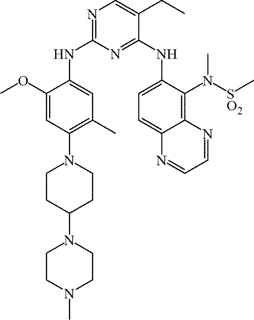 | ¹H NMR (600 MHz, Methanol-d₄) δ 8.99 (d, J = 1.8 Hz, 1H), 8.95 (d, J = 1.9 Hz, 1H), 8.46 (br s, J = 34.6 Hz, 1H), 8.13 - 8.04 (m, 1H), 7.81 - 7.72 (m, 1H), 7.19 (s, 1H), 6.76 (s, 1H), 3.82 (s, 3H), 3.46 - 3.35 (m, 9H), 3.26 - 3.17 (m, 2H), 3.15 (s, 3H), 3.12 - 3.06 (m, 1H), 2.89 (s, 3H), 2.81 -2.74 (m, 2H), 2.72 - 2.67 (m, 2H), 2.19 -2.11 (m, 2H), 1.98 - 1.80 (m, 4H), 1.38 (t, J = 7.4 Hz, 3H). MS (ESI) m/z: 675 [M+H]⁺. |
| I-318 | 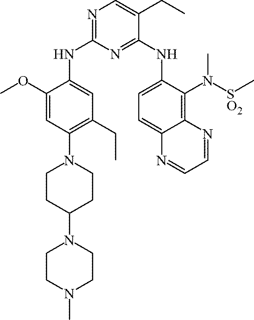 | ¹H NMR (600 MHz, Methanol-d₄) δ 8.99 (d, J = 1.8 Hz, 1H), 8.95 (d, J = 1.8 Hz, 1H), 8.39 (br s, 1H), 8.12 - 8.00 (m, 1H), 7.83 - 7.67 (m, 1H), 7.24 (s, 1H), 6.80 (s, 1H), 3.83 (s, 3H), 3.44 (s, 3H), 3.36 - 3.23 (m, 10H), 3.16 - 3.08 (m, 5H), 3.01 - 2.94 (m, 1H), 2.83 (s, 3H), 2.81 - 2.75 (m, 2H), 2.72 - 2.67 (m, 2H), 2.49 - 2.19 (m, 2H), 2.13 - 2.07 (m, 2H), 1.87 - 1.77 (m, 2H), 1.38 (t, J = 7.4 Hz, 3H), 1.03 - 0.62 (m, 3H). MS (ESI) m/z: 689 [M+H]⁺. |
| I-319 | 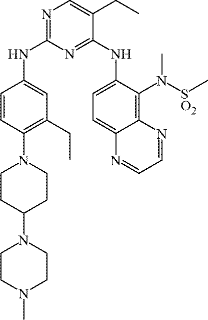 [SMILES: CCc1cc(Nc2ncc(CC)c(Nc3ccc4nccnc4c3N(C)S(C)(=O)=O)n2)ccc1N1CCC(N2CCN(C)CC2)CC1] | ¹H NMR (600 MHz, Methanol-d₄) δ 8.99 (d, J = 1.8 Hz, 1H), 8.95 (d, J = 1.9 Hz, 1H), 8.47 - 8.37 (m, 1H), 8.05 (d, J = 9.2 Hz, 1H), 7.77 (s, 1H), 7.20 (d, J = 2.6 Hz, 1H), 7.10 (dd, J = 8.5, 2.6 Hz, 1H), 7.00 (d, J = 8.6 Hz, 1H), 3.41 (s, 3H), 3.39 - 3.36 (m, 1H), 3.23 - 3.05 (m, 12H), 2.88 - 2.82 (m, 1H), 2.78 (s, 3H), 2.73 - 2.68 (m, 4H), 2.55 - 2.36 (m, 2H), 2.10 - 2.01 (m, 2H), 1.83 - 1.73 (m, 2H), 1.38 (t, J = 7.5 Hz, 4H), 1.00 - 0.91 (m, 3H). MS (ESI) m/z: 659 [M+H]⁺. |
| I-320 | 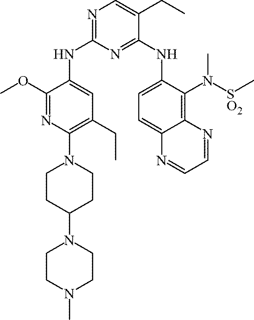 | ¹H NMR (600 MHz, Methanol-d₄) δ 9.00 (d, J = 1.9 Hz, 1H), 8.96 (d, J = 1.9 Hz, 1H), 8.31 (br s, 1H), 8.06 (d, J = 9.0 Hz, 1H), 7.86 - 7.76 (m, 1H), 7.54 (s, 1H), 3.91 (s, 3H), 3.52 - 3.40 (m, 6H), 3.39 - 3.23 (m, 7H), 3.14 (s, 3H), 3.08 - 3.02 (m, 1H), 2.92 - 2.86 (m, 2H), 2.84 (s, 3H), 2.70 (q, J = 7.4 Hz, 2H), 2.37 - 2.23 (m, 1H), 2.18 - 2.03 (m, 3H), 1.88 - 1.77 (m, 2H), 1.38 (t, J = 7.5 Hz, 3H), 0.93 - 0.52 (m, 3H). MS (ESI) m/z: 690 [M+H]⁺. |
| I-321 | 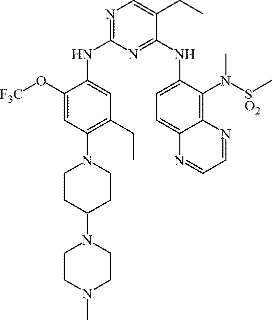 [SMILES: CCc1cc(Nc2ncc(CC)c(Nc3ccc4nccnc4c3N(C)S(C)(=O)=O)n2)c(OC(F)(F)F)cc1N1CCC(N2CCN(C)CC2)CC1] | ¹H NMR (600 MHz, Methanol-d₄) δ 8.98 (d, J = 1.9 Hz, 1H), 8.94 (d, J = 1.9 Hz, 1H), 8.22 (d, J = 9.1 Hz, 1H), 7.95 (d, J = 9.2 Hz, 1H), 7.84 (s, 1H), 7.35 (s, 1H), 7.03 (s, 1H), 3.41 (s, 3H), 3.38 - 3.17 (m, 8H), 3.13 (s, 3H), 3.11 - 3.04 (m, 2H), 2.97 - 2.91 (m, 1H), 2.85 (s, 3H), 2.70 (q, J = 7.4 Hz, 4H), 2.50 - 2.39 (m, 1H), 2.35 -2.25 (m, 1H), 2.14 - 2.05 (m, 2H), 1.87 - 1.76 (m, 2H), 1.39 (t, J = 7.5 Hz, 3H), 0.91 - 0.78 (m, 3H). MS (ESI) m/z: 743 [M+H]⁺. |
| I-322 | 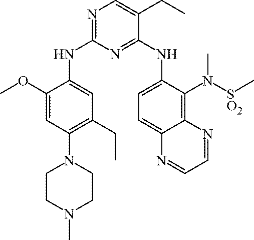 | ¹H NMR (600 MHz, Methanol-d₄) δ 9.00 (d, J = 1.9 Hz, 1H), 8.95 (d, J = 1.9 Hz, 1H), 8.38 (s, 1H), 8.06 (d, J = 9.2 Hz, 1H), 7.81 (s, 1H), 7.34 (s, 1H), 6.83 (s, 1H), 3.85 (s, 3H), 3.66 - 3.57 (m, 2H), 3.45 (s, 3H), 3.31 - 3.26 (m, 2H), 3.20 - 3.11 (m, 7H), 2.99 (s, 3H), 2.70 (q, J = 7.4 Hz, 2H), 2.42 - 2.19 (m, 2H), 1.38 (t, J = 7.4 Hz, 3H), 0.92 - 0.62 (m, 3H). MS (ESI) m/z: 606 [M+H]⁺. |
| I-323 | 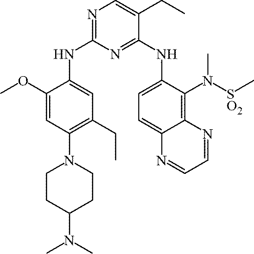 | ¹H NMR (600 MHz, Methanol-d₄) δ 8.99 (d, J = 1.9 Hz, 1H), 8.95 (d, J = 1.8 Hz, 1H), 8.46 - 8.33 (m, 1H), 8.10 - 8.01 (m, 1H), 7.82 - 7.72 (m, 1H), 7.28 (s, 1H), 6.80 (s, 1H), 3.83 (s, 3H), 3.44 (s, 3H), 3.35 - 3.32 (m, 1H), 3.19 - 3.12 (m, 5H), 2.93 (s, 6H), 2.84 - 2.78 (m, 2H), 2.69 (q, J = 7.5 Hz, 2H), 2.42 - 2.25 (m, 2H), 2.20 - 2.14 (m, 2H), 1.95 - 1.86 (m, 2H), 1.38 (t, J = 7.5 Hz, 3H), 0.92 - 0.74 (m, 3H). MS (ESI) m/z: 634 [M+H]⁺. |
| I-324 | 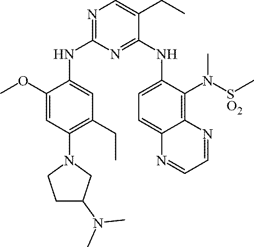 | ¹H NMR (600 MHz, Methanol-d₄) δ 8.99 (d, J = 1.9 Hz, 1H), 8.94 (d, J = 1.9 Hz, 1H), 8.41 (br s, 1H), 8.05 (d, J = 9.2 Hz, 1H), 7.84 - 7.74 (m, 1H), 7.26 (s, 1H), 6.79 (d, J = 2.1 Hz, 1H), 4.07 - 3.96 (m, 1H), 3.84 (s, 3H), 3.46 (s, 3H), 3.42 - 3.38 (m, 2H), 3.31 - 3.27 (m, 1H), 3.14 (s, 3H), 3.12 - 3.07 (m, 1H), 2.97 (d, J = 2.1 Hz, 6H), 2.71 - 2.67 (m, 2H), 2.52 - 2.45 (m, 1H), 2.43 - 2.25 (m, 2H), 2.25 - 2.15 (m, 1H), 1.38 (t, J = 7.5 Hz, 3H), 0.94 - 0.67 (m, 3H). MS (ESI) m/z: 620 [M+H]⁺. |
| I-325 | 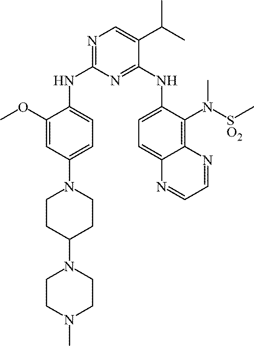 | ¹H NMR (600 MHz, Methanol-d₄) δ 8.99 (d, J = 1.8 Hz, 1H), 8.95 (d, J = 1.9 Hz, 1H), 8.57 - 8.42 (m, 1H), 8.11 (d, J = 9.3 Hz, 1H), 7.83 - 7.67 (m, 1H), 7.27 (d, J = 8.0 Hz, 1H), 6.78 (s, 1H), 6.60 - 6.44 (m, 1H), 3.86 - 3.79 (m, 5H), 3.49 - 3.36 (m, 7H), 3.31 - 3.23 (m, 4H), 3.15 (s, 3H), 3.12 - 3.03 (m, 2H), 2.98 - 2.91 (m, 2H), 2.89 (s, 3H), 2.19 - 2.07 (m, 2H), 1.90 - 1.78 (m, 2H), 1.42 (d, J = 6.8 Hz, 3H), 1.39 (d, J = 6.8 Hz, 3H). MS (ESI) m/z: 675 [M+H]⁺. |
| I-326 | 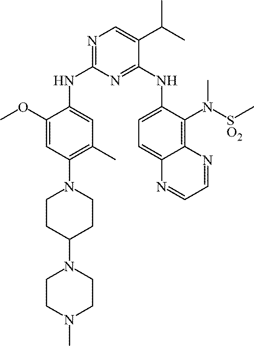 | ¹H NMR (600 MHz, Methanol-d₄) δ 9.00 (d, J = 1.9 Hz, 1H), 8.95 (d, J = 1.9 Hz, 1H), 8.46 - 8.25 (m, 1H), 8.11 - 8.01 (m, 1H), 7.83 - 7.74 (m, 1H), 7.24 (s, 1H), 6.79 (s, 1H), 3.82 (s, 3H), 3.43 (s, 3H), 3.31 - 3.16 (m, 7H), 3.14 (s, 3H), 3.13 -3.06 (m, 4H), 2.98 - 2.88 (m, 1H), 2.81 (s, 3H), 2.80 - 2.73 (m, 2H), 2.47 - 2.16 (m, 2H), 2.13 - 2.01 (m, 2H), 1.84 - 1.73 (m, 2H), 1.42 (d, J = 6.8 Hz, 3H), 1.40 (d, J = 6.8 Hz, 3H), 1.00 - 0.48 (m, 3H). MS (ESI) m/z: 689 [M+H]⁺. |
| I-327 | 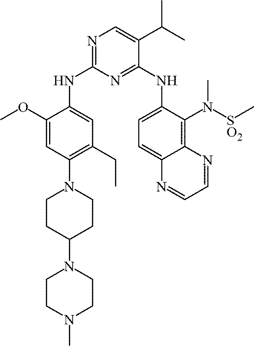 | ¹H NMR (600 MHz, Methanol-d₄) δ 9.00 (d, J = 1.9 Hz, 1H), 8.96 (d, J = 1.9 Hz, 1H), 8.40 - 8.18 (m, 1H), 8.05 (d, J = 9.2 Hz, 1H), 7.91 - 7.74 (m, 1H), 7.54 (s, 1H), 3.49 - 3.40 (m, 5H), 3.34 - 3.15 (m, 8H), 3.14 (s, 3H), 3.11 - 3.06 (m, 1H), 3.01 - 2.94 (m, 1H), 2.91 - 2.83 (m, 2H), 2.80 (s, 3H), 2.36 - 2.10 (m, 2H), 2.10 - 2.03 (m, 2H), 1.84 - 1.74 (m, 2H), 1.42 (d, J = 6.8 Hz, 3H), 1.40 (d, J = 6.8 Hz, 3H), 0.73 (s, 3H). MS (ESI) m/z: 703 [M+H]⁺. |
| I-328 | 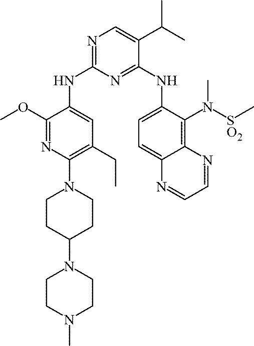 | ¹H NMR (600 MHz, Methanol-d₄) δ 9.00 (d, J = 1.9 Hz, 1H), 8.96 (d, J = 1.9 Hz, 1H), 8.40 - 8.18 (m, 1H), 8.05 (d, J = 9.2 Hz, 1H), 7.91 - 7.74 (m, 1H), 7.54 (s, 1H), 3.49 - 3.40 (m, 5H), 3.34 - 3.15 (m, 8H), 3.14 (s, 3H), 3.11 - 3.06 (m, 1H), 3.01 - 2.94 (m, 1H), 2.91 - 2.83 (m, 2H), 2.80 (s, 3H), 2.36 - 2.10 (m, 2H), 2.10 - 2.03 (m, 2H), 1.84 - 1.74 (m, 2H), 1.42 (d, J = 6.8 Hz, 3H), 1.40 (d, J = 6.8 Hz, 3H), 0.73 (s, 3H). MS (ESI) m/z: 704 [M+H]⁺. |
| I-329 | 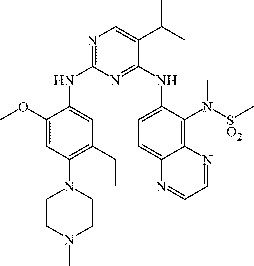 | ¹H NMR (600 MHz, Methanol-d₄) δ 8.99 (d, J = 1.8 Hz, 1H), 8.94 (d, J = 1.8 Hz, 1H), 8.39 (d, J = 9.0 Hz, 1H), 8.05 (d, J = 9.2 Hz, 1H), 7.85 (s, 1H), 7.39 (s, 1H), 6.82 (s, 1H), 3.86 (s, 3H), 3.70 - 3.51 (m, 2H), 3.45 (s, 3H), 3.31 - 3.21 (m, 2H), 3.16 - 3.11 (m, 7H), 3.08 (q, J = 6.8 Hz, 1H), 2.98 (s, 3H), 2.42 - 2.15 (m, 2H), 1.42 (d, J = 6.8 Hz, 3H), 1.40 (d, J = 6.8 Hz, 3H), 0.87 - 0.65 (m, 3H). MS (ESI) m/z: 620 [M+H]⁺. |
| I-330 | 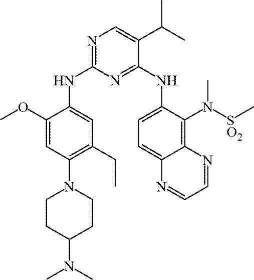 | ¹H NMR (600 MHz, Methanol-d₄) δ 9.00 (d, J = 1.9 Hz, 1H), 8.95 (d, J = 1.9 Hz, 1H), 8.51 - 8.21 (m, 1H), 8.06 (d, J = 9.2 Hz, 1H), 7.79 (s, 1H), 7.27 (s, 1H), 6.80 (s, 1H), 3.83 (s, 3H), 3.44 (s, 3H), 3.35 - 3.32 (m, 1H), 3.17 - 3.06 (m, 6H), 2.93 (s, 6H), 2.85 - 2.75 (m, 2H), 2.48 - 2.21 (m, 2H), 2.22 - 2.15 (m, 2H), 1.95 - 1.83 (m, 2H), 1.42 (d, J = 6.8 Hz, 3H), 1.40 (d, J = 6.7 Hz, 3H), 0.97 - 0.50 (m, 3H). MS (ESI) m/z: 648 [M+H]⁺. |
| I-331 | 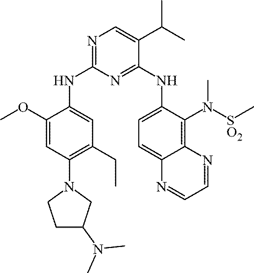 | ¹H NMR (600 MHz, Methanol-d₄) δ 9.00 (d, J = 1.8 Hz, 1H), 8.95 (d, J = 1.8 Hz, 1H), 8.46 - 8.26 (m, 1H), 8.13 - 7.97 (m, 1H), 7.80 (s, 1H), 7.25 (s, 1H), 6.79 (d, J = 2.8 Hz, 1H), 4.05 - 3.96 (m, 1H), 3.84 (s, 3H), 3.45 (s, 3H), 3.42 - 3.38 (m, 2H), 3.30 - 3.27 (m, 1H), 3.14 (s, 3H), 3.12 -3.05 (m, 2H), 2.97 (d, J = 2.0 Hz, 6H), 2.51 - 2.43 (m, 1H), 2.42 - 2.25 (m, 2H), 2.24 -2.16 (m, 1H), 1.42 (d, J = 6.7 Hz, 3H), 1.40 (d, J = 6.8 Hz, 3H), 0.97 - 0.57 (m, 3H). MS (ESI) m/z: 634 [M+H]⁺. |
| I-332 | 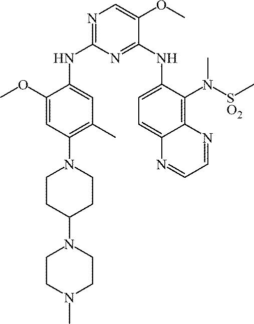 | ¹H NMR (600 MHz, Methanol-d₄) δ 8.99 (d, J = 1.9 Hz, 1H), 8.92 (d, J = 1.9 Hz, 1H), 8.71 (d, J = 8.0 Hz, 1H), 8.08 (d, J = 9.3 Hz, 1H), 7.59 (s, 1H), 7.26 (s, 1H), 6.80 (s, 1H), 4.01 (s, 3H), 3.83 (s, 3H), 3.47 (s, 3H), 3.30 - 3.00 (m, 13H), 2.96 - 2.87 (m, 1H), 2.84 - 2.74 (m, 5H), 2.14 - 2.07 (m, 5H), 1.89 - 1.76 (m, 2H). MS (ESI) m/z: 677 [M+H]⁺. |
| I-333 | 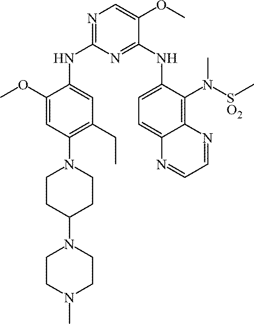 | ¹H NMR (600 MHz, Methanol-d₄) δ 8.99 (d, J = 1.8 Hz, 1H), 8.93 (d, J = 1.9 Hz, 1H), 8.74 - 8.56 (m, 1H), 8.08 (d, J = 9.4 Hz, 1H), 7.59 (s, 1H), 7.29 (s, 1H), 6.84 (s, 1H), 4.02 (s, 3H), 3.84 (s, 3H), 3.47 (s, 3H), 3.30 - 2.97 (m, 13H), 2.91 - 2.76 (m, 6H), 2.57 - 2.38 (m, 2H), 2.13 - 2.03 (m, 2H), 1.85 - 1.72 (m, 2H), 1.04 - 0.86 (m, 3H). MS (ESI) m/z: 691 [M+H]⁺. |
| I-334 | 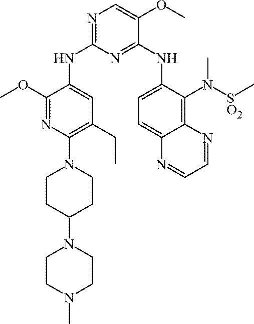 | ¹H NMR (600 MHz, Methanol-d₄) δ 8.99 (d, J = 1.9 Hz, 1H), 8.93 (d, J = 1.9 Hz, 1H), 8.61 - 8.50 (m, 1H), 8.06 (d, J = 9.3 Hz, 1H), 7.66 (s, 1H), 7.62 (s, 1H), 4.02 (s, 3H), 3.92 (s, 3H), 3.55 - 3.49 (m, 2H), 3.47 (s, 3H), 3.37 - 3.23 (m, 8H), 3.17 (s, 3H), 3.09 - 3.00 (m, 1H), 2.96 - 2.88 (m, 2H), 2.83 (s, 3H), 2.47 - 2.27 (m, 2H), 2.16 - 2.03 (m, 2H), 1.94 - 1.78 (m, 2H), 0.89 (s, 3H). MS (ESI) m/z: 692 [M+H]⁺. |
| I-335 | 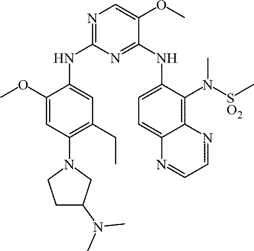 | ¹H NMR (600 MHz, Methanol-d₄) δ 8.97 (d, J = 1.8 Hz, 1H), 8.90 (d, J = 1.8 Hz, 1H), 8.69 (d, J = 9.3 Hz, 1H), 8.03 (d, J = 9.3 Hz, 1H), 7.65 (s, 1H), 7.38 (s, 1H), 6.82 (s, 1H), 4.06 - 3.98 (m, 4H), 3.85 (s, 3H), 3.47 (s, 3H), 3.45 - 3.40 (m, 2H), 3.35 - 3.32 (m, 1H), 3.18 (s, 3H), 3.17 - 3.11 (m, 1H), 2.98 (s, 6H), 2.56 - 2.43 (m, 3H), 2.28 - 2.16 (m, 1H), 1.00 - 0.88 (m, 3H). MS (ESI) m/z: 622 [M+H]⁺. |
| I-336 | 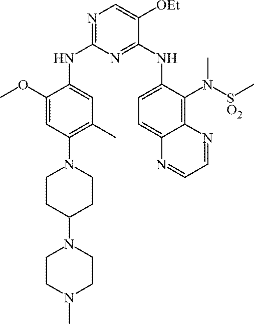 | ¹H NMR (600 MHz, Methanol-d₄) δ 8.97 (d, J = 1.9 Hz, 1H), 8.92 (d, J = 9.3 Hz, 1H), 8.89 (d, J = 1.9 Hz, 1H), 8.06 (d, J = 9.4 Hz, 1H), 7.60 (s, 1H), 7.36 (s, 1H), 6.80 (s, 1H), 4.24 - 4.15 (m, 2H), 3.84 (s, 3H), 3.48 (s, 3H), 3.30 - 3.22 (m, 5H), 3.21 (s, 3H), 3.18 -2.68 (m, 11H), 2.18 (s, 3H), 2.10 - 2.04 (m, 2H), 1.85 - 1.72 (m, 2H), 1.55 (t, J = 6.9 Hz, 3H). MS (ESI) m/z: 691 [M+H]⁺. |
| I-337 | 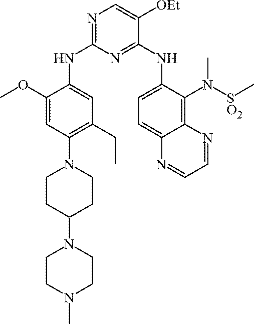 | ¹H NMR (600 MHz, Methanol-d₄) δ 8.98 (d, J = 1.8 Hz, 1H), 8.90 (d, J = 1.9 Hz, 1H), 8.86 (d, J = 9.6 Hz, 1H), 8.05 (d, J = 9.4 Hz, 1H), 7.60 (s, 1H), 7.37 (s, 1H), 6.86 (s, 1H), 4.22 - 4.13 (m, 2H), 3.84 (s, 3H), 3.48 (s, 3H), 3.36 - 3.22 (m, 3H), 3.20 (s, 3H), 3.18 - 3.15 (m, 2H), 3.14 - 2.87 (m, 4H), 2.87 - 2.78 (m, 4H), 2.76 (s, 3H), 2.60 - 2.53 (m, 2H), 2.13 - 2.01 (m, 2H), 1.87 - 1.75 (m, 2H), 1.55 (t, J = 6.9 Hz, 3H), 1.03 (t, J = 7.5 Hz, 3H). MS (ESI) m/z: 705 [M+H]⁺. |
| I-338 | 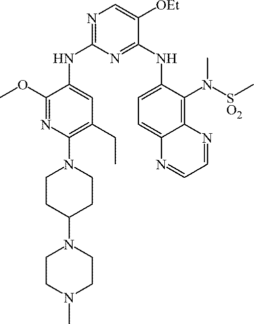 | ¹H NMR (600 MHz, Methanol-d₄) δ 8.97 (d, J = 1.8 Hz, 1H), 8.89 (d, J = 1.9 Hz, 1H), 8.79 (d, J = 9.4 Hz, 1H), 8.03 (d, J = 9.3 Hz, 1H), 7.73 (s, 1H), 7.66 (s, 1H), 4.22 - 4.14 (m, 2H), 3.93 (s, 3H), 3.53 -3.46 (m, 5H), 3.26 - 3.21 (m, 1H), 3.20 (s, 3H), 3.18 - 2.79 (m, 10H), 2.75 (s, 3H), 2.55 - 2.46 (m, 2H), 2.09 - 2.03 (m, 2H), 1.83 - 1.73 (m, 2H), 1.54 (t, J = 6.9 Hz, 3H), 1.01 (t, J = 7.5 Hz, 3H). MS (ESI) m/z: 706 [M+H]⁺. |
| I-339 | 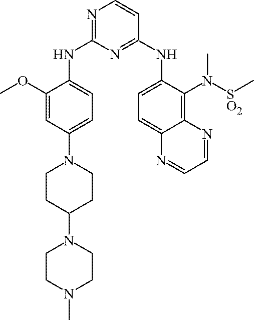 | MS (ESI) m/z: 633 [M+H]⁺. |
| I-340 | 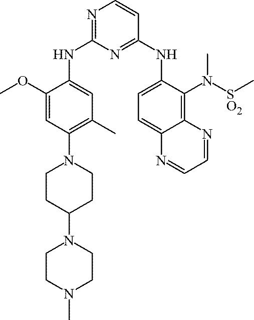 | ¹H NMR (600 MHz, Methanol-d₄) δ 8.98 (d, J = 1.9 Hz, 1H), 8.93 (d, J = 1.9 Hz, 1H), 8.49 - 8.15 (m, 1H), 8.14 - 7.99 (m, 1H), 7.96 - 7.80 (m, 1H), 7.34 - 7.17 (m, 1H), 6.76 (s, 1H), 6.67 (d, J = 7.2 Hz, 1H), 3.82 (s, 3H), 3.63 - 3.37 (m, 11H), 3.27 - 3.16 (m, 3H), 3.14 (s, 3H), 2.92 (s, 3H), 2.83 - 2.73 (m, 2H), 2.21 - 2.13 (m, 2H), 2.05 - 1.71 (m, 5H). MS (ESI) m/z: 647 [M+H]⁺. |
| I-341 | 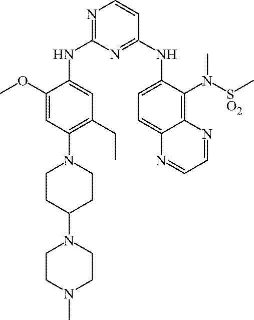 | ¹H NMR (600 MHz, Methanol-d₄) δ 8.98 (d, J = 1.8 Hz, 1H), 8.93 (d, J = 1.9 Hz, 1H), 8.42 - 8.11 (m, 1H), 8.11 - 7.97 (m, 1H), 7.96 - 7.83 (m, 1H), 7.30 (s, 1H), 6.79 (s, 1H), 6.66 (d, J = 7.2 Hz, 1H), 3.83 (s, 3H), 3.58 - 3.40 (m, 11H), 3.20 - 3.10 (m, 6H), 2.91 (s, 3H), 2.85 - 2.76 (m, 2H), 2.21 - 2.10 (m, 2H), 1.94 - 1.80 (m, 2H), 1.42 - 1.35 (m, 2H), 1.07 - 0.33 (m, 3H). MS (ESI) m/z: 661 [M+H]⁺. |
| I-342 | 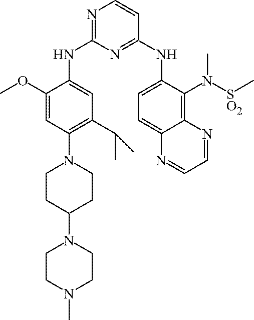 | ¹H NMR (600 MHz, Methanol-d₄) δ 8.97 (d, J = 1.9 Hz, 1H), 8.92 (d, J = 1.8 Hz, 1H), 8.31 - 8.08 (m, 1H), 8.07 - 7.94 (m, 1H), 7.93 - 7.80 (m, 1H), 7.24 (s, 1H), 6.81 (s, 1H), 6.65 (d, J = 7.2 Hz, 1H), 3.81 (s, 3H), 3.47 (s, 3H), 3.39 - 3.18 (m, 9H), 3.12 (s, 3H), 3.09 - 3.02 (m, 2H), 3.01 - 2.94 (m, 1H), 2.84 - 2.77 (m, 5H), 2.14 - 2.07 (m, 2H), 1.86 - 1.77 (m, 2H), 1.30 - 0.43 (m, 6H). MS (ESI) m/z: 675 [M+H]⁺. |
| I-343 | 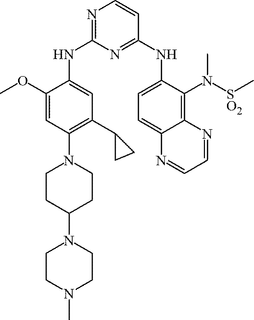 | ¹H NMR (600 MHz, Methanol-d₄) δ 8.98 (d, J = 1.9 Hz, 1H), 8.92 (d, J = 1.8 Hz, 1H), 8.30 - 8.07 (m, 1H), 8.05 - 7.93 (m, 1H), 7.91 - 7.81 (m, 1H), 6.82 (s, 1H), 6.73 (s, 1H), 6.64 (d, J = 7.2 Hz, 1H), 3.83 (s, 3H), 3.49 (s, 3H), 3.44 - 3.38 (m, 2H), 3.31 - 3.22 (m, 8H), 3.13 (s, 3H), 3.02 -2.93 (m, 1H), 2.82 (s, 3H), 2.80 - 2.74 (m, 2H), 2.15 - 2.07 (m, 2H), 2.04 - 1.90 (m, 1H), 1.89 - 1.80 (m, 2H), 1.05 - -0.04 (m, 4H). MS (ESI) m/z: 673 [M+H]⁺. |
| I-344 | 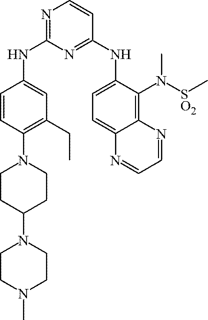 [SMILES: CCc1cc(Nc2nccc(Nc3ccc4nccnc4c3N(C)S(C)(=O)=O)n2)ccc1N1CCC(N2CCN(C)CC2)CC1] | ¹H NMR (600 MHz, Methanol-d₄) δ 8.98 (d, J = 1.8 Hz, 1H), 8.94 (d, J = 1.8 Hz, 1H), 8.34 - 8.15 (m, 1H), 8.02 (d, J = 9.2 Hz, 1H), 7.93 - 7.84 (m, 1H), 7.25 (d, J = 2.5 Hz, 1H), 7.12 (dd, J = 8.4, 2.7 Hz, 1H), 7.07 - 6.95 (m, 1H), 6.67 (d, J = 7.2 Hz, 1H), 3.46 (s, 3H), 3.44 - 3.32 (m, 8H), 3.14 - 3.05 (m, 6H), 2.87 (s, 3H), 2.79 -2.72 (m, 2H), 2.56 - 2.30 (m, 2H), 2.19 -2.09 (m, 2H), 1.90 - 1.80 (m, 2H), 1.06 -0.63 (m, 3H). MS (ESI) m/z: 631 [M+H]⁺. |
| I-345 | 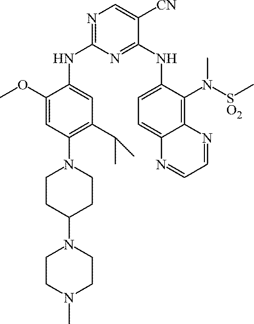 | ¹H NMR (600 MHz, Methanol-d₄) δ 8.94 (d, J = 1.8 Hz, 1H), 8.88 - 8.81 (m, 1H), 8.46 - 8.37 (m, 2H), 7.98 - 7.72 (m, 1H), 7.11 - 6.89 (m, 1H), 6.74 (s, 1H), 3.81 (s, 3H), 3.49 - 3.42 (m, 5H), 3.25 - 3.09 (m, 9H), 2.93 (s, 1H), 2.87 - 2.75 (m, 6H), 2.14 - 2.08 (m, 2H), 2.07 - 1.90 (m, 2H), 1.88 - 1.82 (m, 2H), 0.63 - 0.28 (m, 3H), 0.20 - -0.09 (m, 3H). MS (ESI) m/z: 700 [M+H]⁺. |
| I-346 | 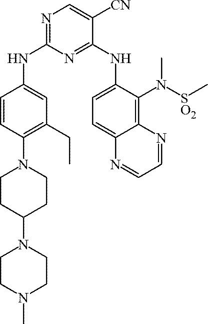 [SMILES: CCc1cc(Nc2ncc(C#N)c(Nc3ccc4nccnc4c3N(C)S(C)(=O)=O)n2)ccc1N1CCC(N2CCN(C)CC2)CC1] | ¹H NMR (600 MHz, Methanol-d₄) δ 8.92 (d, J = 1.8 Hz, 1H), 8.83 - 8.78 (m, 1H), 8.65 - 8.48 (m, 1H), 8.41 (s, 1H), 8.07 - 7.87 (m, 1H), 7.52 - 7.19 (m, 2H), 7.16 - 6.99 (m, 1H), 3.64 - 3.32 (m, 11H), 3.24 -3.16 (m, 5H), 3.14 - 3.07 (m, 1H), 2.97 - 2.84 (m, 5H), 2.64 - 2.36 (m, 2H), 2.22 - 2.09 (m, 2H), 1.99 - 1.86 (m, 2H), 1.19 - 0.77 (m, 3H). MS (ESI) m/z: 656 [M+H]⁺. |
| I-347 | 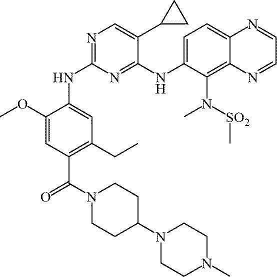 [SMILES: CCc1cc(Nc2ncc(C3CC3)c(Nc3ccc4nccnc4c3N(C)S(C)(=O)=O)n2)c(OC)cc1C(=O)N1CCC(N2CCN(C)CC2)CC1] | ¹H NMR (600 MHz, DMSO-d₆) δ 10.05 (s, 1H), 9.78 - 9.66 (m, 1H), 9.11 - 8.98 (m, 2H), 8.50 (s, 1H), 8.17 - 8.05 (m, 2H), 7.52 - 7.40 (m, 1H), 7.05 - 6.92 (m, 1H), 3.88 - 3.79 (m, 3H), 3.75 - 3.28 (m, 15H), 3.24 - 3.20 (m, 3H), 3.14 - 2.97 (m, 2H), 2.89 (s, 3H), 2.84 - 2.75 (m, 1H), 2.27 - 1.91 (m, 2H), 1.84 - 1.63 (m, 2H), 1.57 - 1.30 (m, 1H), 1.16 - 1.05 (m, 3H), 0.89 - 0.64 (m, 4H). MS (ESI) m/z: 729 [M+H]⁺. |
| I-348 | 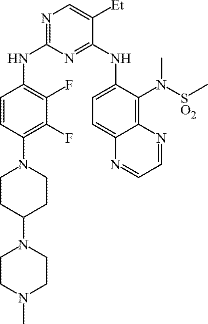 [SMILES: CCc1cnc(Nc2ccc(N3CCC(N4CCN(C)CC4)CC3)c(F)c2F)nc1Nc1ccc2nccnc2c1N(C)S(C)(=O)=O] | ¹H NMR (600 MHz, Methanol-d₄) δ 8.97 (d, J = 1.9 Hz, 1H), 8.92 (d, J = 1.9 Hz, 1H), 8.40 (d, J = 9.3 Hz, 1H), 7.99 (d, J = 9.2 Hz, 1H), 7.85 (s, 1H), 7.13 - 7.06 (m, 1H), 6.73 (t, J = 8.6 Hz, 1H), 3.54 - 3.46 (m, 2H), 3.41 (s, 3H), 3.34 - 3.18 (m, 6H), 3.14 (s, 3H), 3.12 - 2.91 (m, 3H), 2.82 (s, 3H), 2.80 - 2.72 (m, 3H), 2.69 (q, J = 7.6 Hz, 2H), 2.11 - 1.98 (m, 2H), 1.84 - 1.70 (m, 2H), 1.38 (t, J = 7.4 Hz, 3H). MS (ESI) m/z: 667 [M+H]⁺. |
| I-349 | 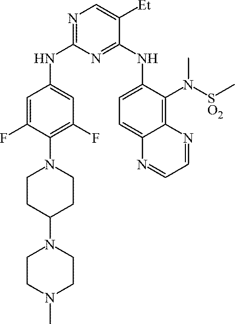 [SMILES: CCc1cnc(Nc2cc(F)c(N3CCC(N4CCN(C)CC4)CC3)c(F)c2)nc1Nc1ccc2nccnc2c1N(C)S(C)(=O)=O] | ¹H NMR (600 MHz, Methanol-d₄) δ 9.00 (d, J = 1.8 Hz, 1H), 8.97 (d, J = 1.8 Hz, 1H), 8.35 (d, J = 9.2 Hz, 1H), 8.15 (d, J = 9.1 Hz, 1H), 7.87 (s, 1H), 6.97 (d, J= 10.7 Hz, 2H), 3.43 (s, 3H), 3.40 - 3.23 (m, 8H), 3.18 - 3.13 (m, 5H), 3.06 (t, J = 11.8 Hz, 2H), 3.01 - 2.95 (m, 1H), 2.85 (s, 3H), 2.71 (q, J = 7.4 Hz, 2H), 2.06 - 1.96 (m, 2H), 1.82 - 1.71 (m, 2H), 1.39 (d, J = 7.5 Hz, 3H). MS (ESI) m/z: 667 [M+H]⁺. |
| I-350 | 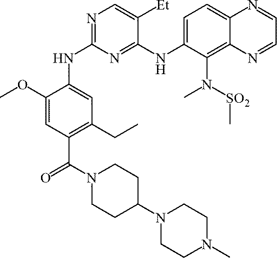 [SMILES: CCc1cc(Nc2ncc(CC)c(Nc3ccc4nccnc4c3N(C)S(C)(=O)=O)n2)c(OC)cc1C(=O)N1CCC(N2CCN(C)CC2)CC1] | ¹H NMR (600 MHz, Methanol-d₄) δ 9.01 (d, J = 2.0 Hz, 1H), 8.97 (d, J = 1.8 Hz, 1H), 8.33 (d, J = 12.0 Hz, 1H), 8.18 - 8.05 (m, 1H), 7.89 (d, J = 9.6 Hz, 1H), 7.53 - 7.46 (m, 1H), 6.90 - 6.79 (m, 1H), 3.91 - 3.84 (m, 3H), 3.49 - 3.43 (m, 3H), 3.43 - 3.18 (m, 8H), 3.14 (s, 3H), 3.12 - 2.88 (m, 5H), 2.88 - 2.85 (m, 3H), 2.75 - 2.69 (m, 2H), 2.09 - 2.00 (m, 2H), 1.99 - 1.79 (m, 2H), 1.66 - 1.47 (m, 2H), 1.40 (t, J = 7.4 Hz, 3H), 0.70 - 0.46 (m, 3H). MS (ESI) m/z: 717 [M+H]⁺. |
| I-351 | 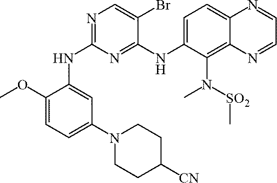 [SMILES: COc1ccc(N2CCC(C#N)CC2)cc1Nc1ncc(Br)c(Nc2ccc3nccnc3c2N(C)S(C)(=O)=O)n1] | ¹H NMR (600 MHz, Methanol-d₄) δ 8.97 (d, J = 1.9 Hz, 1H), 8.90 (d, J = 1.9 Hz, 1H), 8.81 (d, J = 9.4 Hz, 1H), 8.33 (s, 1H), 8.10 (d, J = 9.4 Hz, 1H), 7.96 (s, 1H), 7.08 - 7.04 (m, 1H), 3.91 (s, 3H), 3.50 (s, 3H), 3.21 (s, 3H), 3.20 - 3.14 (m, 2H), 3.13 - 3.00 (m, 2H), 2.88 - 2.80 (m, 1H), 2.06 - 1.95 (m, 2H), 1.92 - 1.76 (m, 2H). MS (ESI) m/z: 638 [M+H]⁺. |
| I-352 | 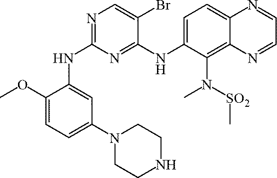 [SMILES: COc1ccc(N2CCNCC2)cc1Nc1ncc(Br)c(Nc2ccc3nccnc3c2N(C)S(C)(=O)=O)n1] | ¹H NMR (600 MHz, Chloroform-d) δ 9.61 (s, 1H), 9.01 (d, J = 9.1 Hz, 1H), 8.87 - 8.79 (m, 3H), 8.32 (s, 1H), 8.13 (d, J = 9.3 Hz, 1H), 8.10 - 8.02 (m, 1H), 7.63 (s, 1H), 6.83 (d, J = 8.7 Hz, 1H), 6.56 (dd, J = 8.6, 2.8 Hz, 1H), 3.89 (s, 3H), 3.51 (s, 3H), 3.24 - 3.10 (m, 11H). MS (ESI) m/z: 614 [M+H]⁺. |
| I-353 | 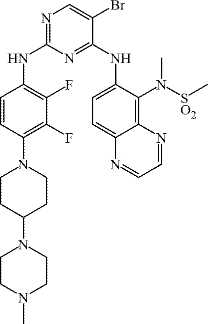 [SMILES: CN1CCN(C2CCN(c3ccc(Nc4ncc(Br)c(Nc5ccc6nccnc6c5N(C)S(C)(=O)=O)n4)c(F)c3F)CC2)CC1] | ¹H NMR (600 MHz, DMSO-d₆) δ 9.33 (s, 1H), 8.98 (d, J = 1.9 Hz, 1H), 8.92 (d, J = 1.9 Hz, 1H), 8.91 - 8.81 (m, 1H), 8.63 (s, 1H), 8.35 (s, 1H), 7.92 (s, 1H), 7.22 (t, J = 8.7 Hz, 1H), 6.91 - 6.82 (m, 1H), 3.65 - 3.36 (m, 11H), 3.25 (s, 3H), 3.13 - 2.89 (m, 3H), 2.81 - 2.71 (m, 5H), 2.09 - 1.99 (m, 2H), 1.77 - 1.66 (m, 2H). MS (ESI) m/z: 717 [M+H]⁺. |
| I-354 | 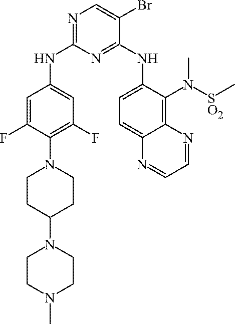 [SMILES: CN1CCN(C2CCN(c3c(F)cc(Nc4ncc(Br)c(Nc5ccc6nccnc6c5N(C)S(C)(=O)=O)n4)cc3F)CC2)CC1] | ¹H NMR (600 MHz, DMSO-d₆) δ 9.84 (s, 1H), 9.01 (d, J = 1.9 Hz, 1H), 8.96 (d, J = 1.9 Hz, 1H), 8.90 - 8.78 (m, 1H), 8.70 (s, 1H), 8.43 (s, 1H), 8.14 (d, J = 9.3 Hz, 1H), 7.40 - 7.30 (m, 2H), 3.93 - 3.28 (m, 11H), 3.23 (s, 3H), 3.15 - 3.11 (m, 2H), 3.01 (t, J = 11.8 Hz, 3H), 2.78 (s, 3H), 2.01 - 1.92 (m, 2H), 1.69 - 1.56 (m, 2H). MS (ESI) m/z: 717 [M+H]⁺. |
| I-355 | 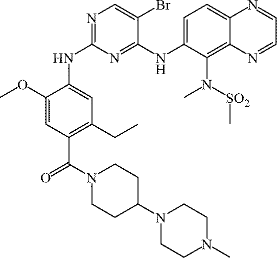 [SMILES: CCc1cc(Nc2ncc(Br)c(Nc3ccc4nccnc4c3N(C)S(C)(=O)=O)n2)c(OC)cc1C(=O)N1CCC(N2CCN(C)CC2)CC1] | ¹H NMR (600 MHz, DMSO-d₆) δ 8.99 (d, J = 1.8 Hz, 1H), 8.95 - 8.92 (m, 1H), 8.81 - 8.74 (m, 1H), 8.72 - 8.69 (m, 1H), 8.61 - 8.54 (m, 1H), 8.39 (s, 1H), 8.05 - 7.99 (m, 1H), 7.63 - 7.55 (m, 1H), 6.94 - 6.85 (m, 1H), 3.87 - 3.74 (m, 3H), 3.69 - 3.42 (m, 8H), 3.38 (s, 3H), 3.23 (s, 3H), 3.20 - 2.93 (m, 4H), 2.83 (s, 3H), 2.80 - 2.71 (m, 1H), 2.42 - 2.18 (m, 2H), 2.13 - 1.80 (m, 2H), 1.66 - 1.22 (m, 2H), 0.90 - 0.74 (m, 3H). MS (ESI) m/z: 767 [M+H]⁺. |
| I-356 | 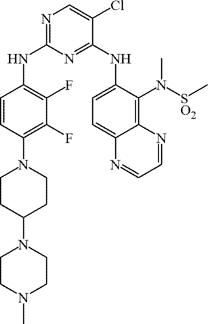 [SMILES: CN1CCN(C2CCN(c3ccc(Nc4ncc(Cl)c(Nc5ccc6nccnc6c5N(C)S(C)(=O)=O)n4)c(F)c3F)CC2)CC1] | ¹H NMR (600 MHz, DMSO-d₆) δ 9.31 (s, 1H), 8.98 (d, J = 1.9 Hz, 1H), 8.93 (d, J = 1.9 Hz, 1H), 8.85 (d, J = 29.4 Hz, 1H), 8.66 (s, 1H), 8.29 (s, 1H), 7.93 (d, J = 8.9 Hz, 1H), 7.25 - 7.17 (m, 1H), 6.91 - 6.78 (m, 1H), 3.61 - 3.42 (m, 6H), 3.37 (s, 3H), 3.24 (s, 3H), 3.20 - 2.86 (m, 5H), 2.81 - 2.71 (m, 5H), 2.12 - 1.98 (m, 2H), 1.78 - 1.65 (m, 2H). MS (ESI) m/z: 673 [M+H]⁺. |
| I-357 | 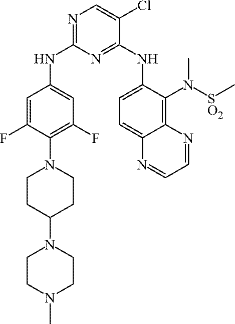 [SMILES: CN1CCN(C2CCN(c3c(F)cc(Nc4ncc(Cl)c(Nc5ccc6nccnc6c5N(C)S(C)(=O)=O)n4)cc3F)CC2)CC1] | ¹H NMR (600 MHz, Methanol-d₄) δ 8.99 (d, J = 9.8 Hz, 1H), 8.95 (s, 1H), 8.84 (s, 1H), 8.19 (s, 1H), 8.10 (d, J = 9.2 Hz, 1H), 7.23 (d, J = 11.9 Hz, 2H), 3.48 (s, 3H), 3.38 - 3.18 (m, 13H), 3.15 - 3.09 (m, 2H), 3.03 - 2.94 (m, 1H), 2.83 (s, 3H), 2.10 -1.96 (m, 2H), 1.84 - 1.72 (m, 2H). MS (ESI) m/z: 673 [M+H]⁺. |
| I-358 | 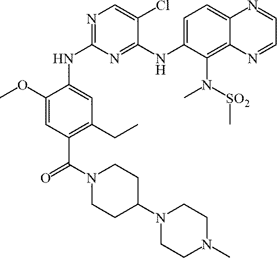 [SMILES: CCc1cc(Nc2ncc(Cl)c(Nc3ccc4nccnc4c3N(C)S(C)(=O)=O)n2)c(OC)cc1C(=O)N1CCC(N2CCN(C)CC2)CC1] | ¹H NMR (600 MHz, Methanol-d₄) δ 8.97 - 8.92 (m, 1H), 8.89 - 8.83 (m, 1H), 8.70 - 8.61 (m, 1H), 8.21 (d, J = 2.8 Hz, 1H), 8.06 (dd, J = 9.1, 5.1 Hz, 1H), 7.85 (d, J = 4.9 Hz, 1H), 6.88 - 6.74 (m, 1H), 3.88 (dd, J = 12.6, 5.0 Hz, 3H), 3.70 - 3.51 (m, 2H), 3.48 - 3.44 (m, 3H), 3.42 - 3.27 (m, 6H), 3.18 (s, 3H), 3.14 - 2.97 (m, 4H), 2.90 - 2.85 (m, 4H), 2.37 - 2.18 (m, 2H), 2.15 - 1.99 (m, 1H), 1.99 - 1.89 (m, 1H), 1.70 - 1.48 (m, 2H), 0.87 - 0.66 (m, 3H). MS (ESI) m/z: 723 [M+H]⁺. |
| I-359 | 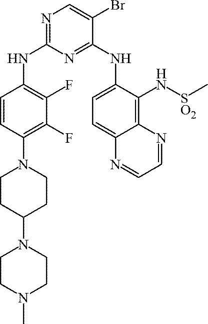 [SMILES: CN1CCN(C2CCN(c3ccc(Nc4ncc(Br)c(Nc5ccc6nccnc6c5NS(C)(=O)=O)n4)c(F)c3F)CC2)CC1] | (IEC200162PUS) ¹H NMR (600 MHz, DMSO-d₆) δ 9.95 (s, 1H), 9.30 (s, 1H), 9.01 (d, J = 1.8 Hz, 1H), 8.94 (s, 1H), 8.93 (d, J = 1.9 Hz, 1H), 8.72 - 8.60 (m, 1H), 8.34 (s, 1H), 7.88 (d, J = 9.4 Hz, 1H), 7.18 (t, J = 8.5 Hz, 1H), 6.88 - 6.77 (m, 1H), 3.87 - 3.59 (m, 8H), 3.55 -3.48 (m, 2H), 3.45 - 3.36 (m, 1H), 3.03 (s, 3H), 2.91 (s, 3H), 2.80 - 2.70 (m, 2H), 2.24 - 2.13 (m, 2H), 1.88 - 1.76 (m, 2H). MS (ESI) m/z: 703 [M+H]⁺. |
| I-360 | 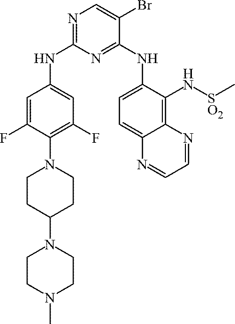 [SMILES: CN1CCN(C2CCN(c3c(F)cc(Nc4ncc(Br)c(Nc5ccc6nccnc6c5NS(C)(=O)=O)n4)cc3F)CC2)CC1] | ¹H NMR (600 MHz, DMSO-d₆) δ 9.99 (s, 1H), 9.82 (s, 1H), 9.05 (d, J = 1.9 Hz, 1H), 9.03 (s, 1H), 8.99 (d, J = 1.8 Hz, 1H), 8.72 - 8.61 (m, 1H), 8.43 (s, 1H), 8.11 (d, J = 9.2 Hz, 1H), 7.36 - 7.28 (m, 2H), 3.80 -3.24 (m, 9H), 3.22 - 3.12 (m, 2H), 3.05 (s, 3H), 3.04 - 2.98 (m, 2H), 2.89 (s, 3H), 2.11 - 2.01 (m, 2H), 1.74 - 1.67 (m, 2H). MS (ESI) m/z: 703 [M+H]⁺. |
| I-361 | 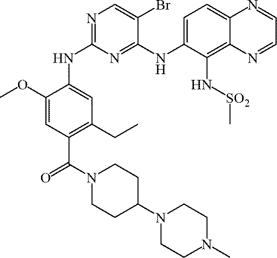 [SMILES: CCc1cc(Nc2ncc(Br)c(Nc3ccc4nccnc4c3NS(C)(=O)=O)n2)c(OC)cc1C(=O)N1CCC(N2CCN(C)CC2)CC1] | ¹H NMR (600 MHz, DMSO-d₆) δ 9.97 (s, 1H), 9.07 - 9.00 (m, 2H), 8.98 - 8.94 (m, 1H), 8.59 (d, J = 8.2 Hz, 1H), 8.46 (s, 1H), 8.40 (s, 1H), 8.06 - 7.97 (m, 1H), 7.67 -7.59 (m, 1H), 6.91 - 6.82 (m, 1H), 3.82 -3.79 (m, 3H), 3.71 - 3.53 (m, 8H), 3.51 -3.38 (m, 2H), 3.35 - 3.23 (m, 2H), 3.04 (s, 3H), 2.86 (s, 3H), 2.81 - 2.72 (m, 1H), 2.36 - 2.04 (m, 2H), 1.99 - 1.85 (m, 2H), 1.69 - 1.42 (m, 2H), 0.81 - 0.68 (m, 3H). MS (ESI) m/z: 753 [M+H]⁺. |
| I-362 | 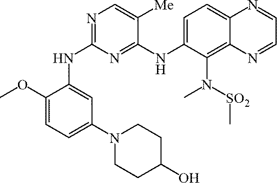 [SMILES: COc1ccc(N2CCC(O)CC2)cc1Nc1ncc(C)c(Nc2ccc3nccnc3c2N(C)S(C)(=O)=O)n1] | ¹H NMR (600 MHz, Methanol-d₄) δ 9.00 (d, J = 1.9 Hz, 1H), 8.95 (d, J = 1.9 Hz, 1H), 8.30 (d, J = 9.3 Hz, 1H), 8.05 (d, J = 9.2 Hz, 1H), 7.93 (s, 1H), 7.74 (d, J = 2.9 Hz, 1H), 7.45 (dd, J = 9.0, 3.0 Hz, 1H), 7.24 (d, J = 9.0 Hz, 1H), 3.92 (s, 3H), 3.79 - 3.69 (m, 1H), 3.49 (s, 3H), 3.46 - 3.37 (m, 1H), 3.34 - 3.31 (m, 1H), 3.18 (s, 3H), 3.16 - 3.11 (m, 1H), 3.09 - 2.96 (m, 1H), 2.32 (s, 3H), 2.00 - 1.91 (m, 1H), 1.88 -1.78 (m, 1H), 1.76 - 1.70 (m, 1H), 1.69 -1.60 (m, 1H). MS (ESI) m/z: 565 [M+H]⁺. |
| I-363 | 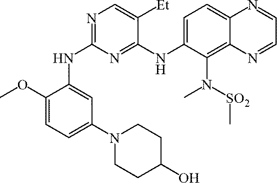 [SMILES: CCc1cnc(Nc2cc(N3CCC(O)CC3)ccc2OC)nc1Nc1ccc2nccnc2c1N(C)S(C)(=O)=O] | ¹H NMR (600 MHz, Methanol-d₄) δ 8.99 (d, J = 1.8 Hz, 1H), 8.94 (d, J = 1.8 Hz, 1H), 8.42 (d, J = 9.3 Hz, 1H), 8.06 (d, J = 9.2 Hz, 1H), 7.91 (s, 1H), 7.69 (s, 1H), 7.23 (dd, J = 9.0, 2.8 Hz, 1H), 7.15 (d, J = 9.0 Hz, 1H), 3.90 (s, 3H), 3.66 - 3.57 (m, 1H), 3.49 (s, 3H), 3.38 - 3.32 (m, 1H), 3.29 - 3.24 (m, 1H), 3.17 (s, 3H), 3.00 -2.91 (m, 1H), 2.91 - 2.83 (m, 1H), 2.71 (q, J = 7.4 Hz, 2H), 1.90 - 1.82 (m, 1H), 1.81 - 1.75 (m, 1H), 1.67 - 1.60 (m, 1H), 1.59 -1.53 (m, 1H), 1.38 (t, J = 7.5 Hz, 3H). MS (ESI) m/z: 579 [M+H]⁺. |
| I-364 | 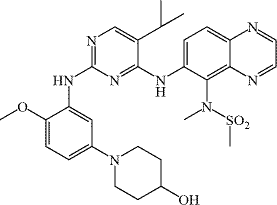 [SMILES: COc1ccc(N2CCC(O)CC2)cc1Nc1ncc(C(C)C)c(Nc2ccc3nccnc3c2N(C)S(C)(=O)=O)n1] | ¹H NMR (600 MHz, Methanol-d₄) δ 8.97 (d, J = 1.9 Hz, 1H), 8.91 (d, J = 1.8 Hz, 1H), 8.43 (d, J = 9.3 Hz, 1H), 8.03 (d, J = 9.2 Hz, 1H), 7.91 (s, 1H), 7.62 (s, 1H), 7.08 (s, 2H), 3.86 (s, 3H), 3.58 - 3.49 (m, 1H), 3.46 (s, 3H), 3.27 - 3.19 (m, 2H), 3.15 (s, 3H), 3.07 (p, J = 6.8 Hz, 1H), 2.84 - 2.69 (m, 2H), 1.84 - 1.66 (m, 2H), 1.60 -1.44 (m, 2H), 1.39 (dd, J = 12.2, 6.8 Hz, 6H). MS (ESI) m/z: 593 [M+H]⁺. |
| I-365 | 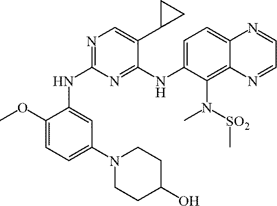 [SMILES: COc1ccc(N2CCC(O)CC2)cc1Nc1ncc(C2CC2)c(Nc2ccc3nccnc3c2N(C)S(C)(=O)=O)n1] | ¹H NMR (600 MHz, Methanol-d₄) δ 9.00 (d, J = 1.9 Hz, 1H), 8.93 (d, J = 1.9 Hz, 1H), 8.61 (d, J = 9.3 Hz, 1H), 8.04 (d, J = 9.3 Hz, 1H), 7.91 (s, 1H), 7.79 (d, J = 2.9 Hz, 1H), 7.41 (dd, J = 9.0, 3.0 Hz, 1H), 7.25 (d, J = 9.0 Hz, 1H), 3.93 (s, 3H), 3.77 - 3.65 (m, 1H), 3.53 (s, 3H), 3.50 - 3.44 (m, 1H), 3.44 - 3.37 (m, 1H), 3.20 (s, 3H), 3.19 - 3.14 (m, 1H), 3.14 - 3.03 (m, 1H), 2.00 - 1.90 (m, 1H), 1.90 - 1.81 (m, 1H), 1.79 - 1.75 (m, 1H), 1.74 - 1.67 (m, 1H), 1.66 - 1.59 (m, 1H), 1.29 - 1.22 (m, 1H), 1.21 - 1.13 (m, 1H), 0.93 - 0.81 (m, 1H), 0.79 - 0.71 (m, 1H). MS (ESI) m/z: 591 [M+H]⁺. |
| I-366 | 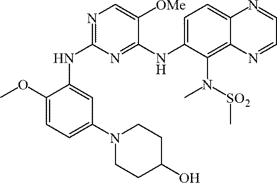 [SMILES: COc1ccc(N2CCC(O)CC2)cc1Nc1ncc(OC)c(Nc2ccc3nccnc3c2N(C)S(C)(=O)=O)n1] | ¹H NMR (600 MHz, Methanol-d₄) δ 8.96 (d, J = 1.8 Hz, 1H), 8.87 (d, J = 1.9 Hz, 1H), 8.75 (d, J = 9.4 Hz, 1H), 8.06 (d, J = 9.3 Hz, 2H), 7.81 (s, 1H), 7.20 (dd, J = 8.8, 2.9 Hz, 1H), 7.14 (d, J = 9.0 Hz, 1H), 4.00 (s, 3H), 3.94 (s, 3H), 3.76 - 3.68 (m, 1H), 3.55 - 3.41 (m, 5H), 3.22 (s, 3H), 3.19 -3.12 (m, 1H), 3.13 - 3.04 (m, 1H), 2.02 -1.92 (m, 1H), 1.91 - 1.84 (m, 1H), 1.77 -1.61 (m, 2H). MS (ESI) m/z: 581 [M+H]⁺. |
| I-367 | 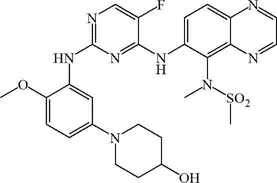 [SMILES: COc1ccc(N2CCC(O)CC2)cc1Nc1ncc(F)c(Nc2ccc3nccnc3c2N(C)S(C)(=O)=O)n1] | ¹H NMR (600 MHz, Methanol-d₄) δ 8.97 (d, J = 1.8 Hz, 1H), 8.89 (d, J = 1.7 Hz, 1H), 8.74 (d, J = 9.5 Hz, 1H), 8.62 - 8.48 (m, 1H), 8.22 (d, J = 3.2 Hz, 1H), 8.13 (d, J = 9.3 Hz, 1H), 7.22 - 7.11 (m, 2H), 3.98 (s, 3H), 3.63 - 3.56 (m, 2H), 3.52 (s, 3H), 3.36 - 3.33 (m, 2H), 3.26 - 3.16 (m, 4H), 2.00 - 1.73 (m, 4H). MS (ESI) m/z: 569 [M+H]⁺. |
| I-368 | 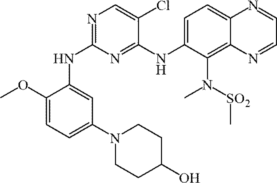 [SMILES: COc1ccc(N2CCC(O)CC2)cc1Nc1ncc(Cl)c(Nc2ccc3nccnc3c2N(C)S(C)(=O)=O)n1] | ¹H NMR (600 MHz, Methanol-d₄) δ 8.95 (d, J = 1.9 Hz, 1H), 8.85 (d, J = 1.8 Hz, 1H), 8.79 (d, J = 9.3 Hz, 1H), 8.44 (s, 1H), 8.26 (s, 1H), 8.04 (d, J = 9.3 Hz, 1H), 7.30 (dd, J = 8.8, 3.0 Hz, 1H), 7.18 (d, J = 8.9 Hz, 1H), 3.97 (s, 3H), 3.93 - 3.84 (m, 1H), 3.67 - 3.61 (m, 1H), 3.60 - 3.54 (m, 1H), 3.50 (s, 3H), 3.45 - 3.38 (m, 1H), 3.37 -3.34 (m, 1H), 3.23 (s, 3H), 2.15 - 2.04 (m, 1H), 2.03 - 1.95 (m, 1H), 1.92 - 1.84 (m, 1H), 1.82 - 1.75 (m, 1H). MS (ESI) m/z: 585 [M+H]⁺. |
| I-369 | 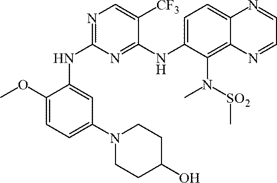 [SMILES: COc1ccc(N2CCC(O)CC2)cc1Nc1ncc(C(F)(F)F)c(Nc2ccc3nccnc3c2N(C)S(C)(=O)=O)n1] | ¹H NMR (600 MHz, Methanol-d₄) δ 8.97 (d, J = 1.9 Hz, 1H), 8.90 (d, J = 1.9 Hz, 1H), 8.74 - 8.59 (m, 1H), 8.53 (s, 1H), 8.50 - 8.32 (m, 1H), 8.08 (d, J = 8.9 Hz, 1H), 7.32 (dd, J = 8.9, 2.9 Hz, 1H), 7.20 (d, J = 8.9 Hz, 1H), 3.96 (s, 3H), 3.90 - 3.79 (m, 1H), 3.68 - 3.53 (m, 2H), 3.49 (s, 3H), 3.41 - 3.34 (m, 2H), 3.19 (s, 3H), 2.13 -1.90 (m, 2H), 1.88 - 1.70 (m, 2H). MS (ESI) m/z: 619 [M+H]⁺. |
| I-370 | 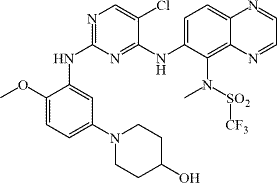 [SMILES: COc1ccc(N2CCC(O)CC2)cc1Nc1ncc(Cl)c(Nc2ccc3nccnc3c2N(C)S(=O)(=O)C(F)(F)F)n1] | ¹H NMR (600 MHz, Methanol-d₄) δ 9.01 (d, J = 1.9 Hz, 1H), 8.94 (d, J = 1.9 Hz, 1H), 8.56 (d, J = 9.4 Hz, 1H), 8.48 - 8.35 (m, 1H), 8.31 (s, 1H), 8.20 (d, J = 9.2 Hz, 1H), 7.17 (d, J = 8.1 Hz, 1H), 7.13 (d, J = 8.8 Hz, 1H), 3.94 (s, 3H), 3.70 - 3.61 (m, 4H), 3.60 - 3.46 (m, 2H), 3.27 - 3.11 (m, 2H), 2.06 - 1.89 (m, 2H), 1.87 - 1.70 (m, 2H). MS (ESI) m/z: 639 [M+H]⁺. |
| I-371 | 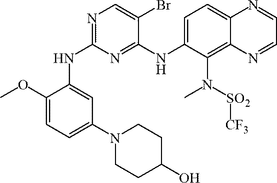 [SMILES: COc1ccc(N2CCC(O)CC2)cc1Nc1ncc(Br)c(Nc2ccc3nccnc3c2N(C)S(=O)(=O)C(F)(F)F)n1] | ¹H NMR (600 MHz, Methanol-d₄) δ 9.00 (d, J = 1.9 Hz, 1H), 8.93 (d, J = 1.9 Hz, 1H), 8.59 (d, J = 9.3 Hz, 1H), 8.45 - 8.39 (m, 2H), 8.18 (d, J = 9.3 Hz, 1H), 7.24 (dd, J = 8.8, 2.9 Hz, 1H), 7.15 (d, J = 8.8 Hz, 1H), 3.94 (s, 3H), 3.83 - 3.72 (m, 1H), 3.64 (s, 3H), 3.63 - 3.50 (m, 2H), 3.36 -3.33 (m, 1H), 3.30 - 3.24 (m, 1H), 2.09 -2.03 (m, 1H), 2.01 - 1.93 (m, 1H), 1.91 -1.83 (m, 1H), 1.82 - 1.72 (m, 1H). MS (ESI) m/z: 683 [M+H]⁺. |
| I-372 | 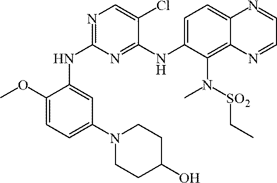 [SMILES: CCS(=O)(=O)N(C)c1c(Nc2nc(Nc3cc(N4CCC(O)CC4)ccc3OC)ncc2Cl)ccc2nccnc12] | ¹H NMR (600 MHz, Methanol-d₄) δ 8.96 (d, J = 1.8 Hz, 1H), 8.86 (d, J = 1.9 Hz, 1H), 8.83 (d, J = 9.3 Hz, 1H), 8.52 (s, 1H), 8.27 (s, 1H), 8.09 (d, J = 9.3 Hz, 1H), 7.22 (dd, J = 8.8, 2.8 Hz, 1H), 7.15 (d, J = 8.8 Hz, 1H), 3.97 (s, 3H), 3.89 - 3.79 (m, 1H), 3.66 - 3.47 (m, 7H), 3.42 - 3.32 (m, 2H), 3.28 - 3.21 (m, 1H), 2.10 - 1.95 (m, 2H), 1.90 - 1.83 (m, 1H), 1.81 - 1.70 (m, 1H), 1.45 (t, J = 7.4 Hz, 3H). MS (ESI) m/z: 599 [M+H]⁺. |
| I-373 | 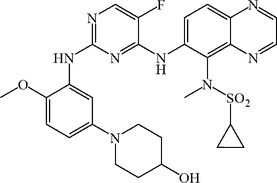 [SMILES: COc1ccc(N2CCC(O)CC2)cc1Nc1ncc(F)c(Nc2ccc3nccnc3c2N(C)S(=O)(=O)C2CC2)n1] | ¹H NMR (600 MHz, Methanol-d₄) δ 8.98 (d, J = 1.8 Hz, 1H), 8.88 (d, J = 1.9 Hz, 1H), 8.66 (d, J = 9.4 Hz, 1H), 8.53 - 8.42 (m, 1H), 8.21 (d, J = 3.3 Hz, 1H), 8.11 (d, J = 9.2 Hz, 1H), 7.22 (dd, J = 8.8, 2.9 Hz, 1H), 7.15 (d, J = 8.9 Hz, 1H), 3.97 (s, 3H), 3.83 - 3.66 (m, 1H), 3.61 - 3.48 (m, 5H), 3.39 - 3.33 (m, 1H), 3.25 - 3.18 (m, 1H), 2.95 - 2.82 (m, 1H), 2.08 - 2.00 (m, 1H), 1.90 - 1.80 (m, 2H), 1.79 - 1.71 (m, 1H), 1.26 - 1.15 (m, 2H), 0.86 - 0.72 (m, 2H). MS (ESI) m/z: 595 [M+H]⁺. |
| I-374 | 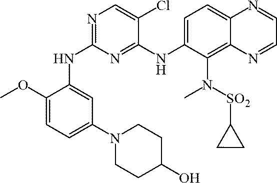 [SMILES: COc1ccc(N2CCC(O)CC2)cc1Nc1ncc(Cl)c(Nc2ccc3nccnc3c2N(C)S(=O)(=O)C2CC2)n1] | ¹H NMR (600 MHz, Methanol-d₄) δ 8.96 (d, J = 1.7 Hz, 1H), 8.85 (d, J = 2.1 Hz, 1H), 8.51 (s, 1H), 8.27 (s, 1H), 8.15 - 8.04 (m, 2H), 7.24 (dd, J = 8.9, 2.8 Hz, 1H), 7.15 (d, J = 8.9 Hz, 1H), 3.97 (s, 3H), 3.87 - 3.75 (m, 1H), 3.65 - 3.57 (m, 2H), 3.54 (s, 3H), 3.44 - 3.35 (m, 2H), 2.95 - 2.91 (m, 1H), 2.10 - 1.96 (m, 2H), 1.88 - 1.75 (m, 2H), 1.24 - 1.19 (m, 2H), 0.77 - 0.71 (m, 2H). MS (ESI) m/z: 611 [M+H]⁺. |
| I-375 | 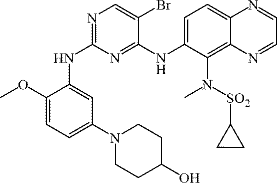 [SMILES: COc1ccc(N2CCC(O)CC2)cc1Nc1ncc(Br)c(Nc2ccc3nccnc3c2N(C)S(=O)(=O)C2CC2)n1] | ¹H NMR (600 MHz, Methanol-d₄) δ 8.96 (d, J = 1.8 Hz, 1H), 8.87 (d, J = 9.2 Hz, 1H), 8.85 (d, J = 1.8 Hz, 1H), 8.49 (s, 1H), 8.36 (s, 1H), 8.09 (d, J = 9.3 Hz, 1H), 7.22 (dd, J = 8.9, 2.8 Hz, 1H), 7.15 (d, J = 8.8 Hz, 1H), 3.97 (s, 3H), 3.89 - 3.76 (m, 1H), 3.63 - 3.54 (m, 5H), 3.37 (s, 2H), 2.99 -2.91 (m, 1H), 2.07 - 1.95 (m, 2H), 1.87 -1.75 (m, 2H), 1.26 - 1.18 (m, 2H), 0.80 -0.74 (m, 2H). MS (ESI) m/z: 655 [M+H]⁺. |
| I-376 | 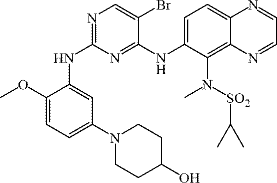 [SMILES: COc1ccc(N2CCC(O)CC2)cc1Nc1ncc(Br)c(Nc2ccc3nccnc3c2N(C)S(=O)(=O)C(C)C)n1] | ¹H NMR (600 MHz, Methanol-d₄) δ 8.98 (d, J = 1.9 Hz, 1H), 8.88 (d, J = 1.9 Hz, 1H), 8.74 (d, J = 9.2 Hz, 1H), 8.49 (s, 1H), 8.38 (s, 1H), 8.10 (d, J = 9.3 Hz, 1H), 7.24 (dd, J = 8.8, 2.9 Hz, 1H), 7.17 (d, J = 8.9 Hz, 1H), 3.97 (s, 3H), 3.87 - 3.77 (m, 1H), 3.64 - 3.47 (m, 7H), 3.40 - 3.36 (m, 1H), 2.07 - 1.98 (m, 2H), 1.87 - 1.74 (m, 2H), 1.70 (d, J = 6.6 Hz, 3H), 1.30 (d, J = 6.9 Hz, 3H). MS (ESI) m/z: 657 [M+H]⁺. |
| I-377 | 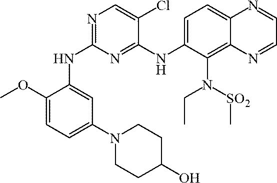 [SMILES: CCN(c1c(Nc2nc(Nc3cc(N4CCC(O)CC4)ccc3OC)ncc2Cl)ccc2nccnc12)S(C)(=O)=O] | ¹H NMR (600 MHz, Methanol-d₄) δ 8.95 -8.93 (m, 1H), 8.92 (d, J = 1.9 Hz, 2H), 8.81 (d, J = 1.9 Hz, 1H), 8.62 - 8.51 (m, 1H), 8.24 (s, 1H), 8.03 (d, J = 9.4 Hz, 1H), 7.26 (dd, J = 8.8, 2.9 Hz, 1H), 7.15 (d, J = 8.9 Hz, 1H), 4.19 -4.11 (m, 1H), 3.98 (s, 3H), 3.93 - 3.87 (m, 1H), 3.85 - 3.79 (m, 1H), 3.73 - 3.63 (m, 2H), 3.49 - 3.40 (m, 2H), 3.22 (s, 3H), 2.14 - 2.03 (m, 2H), 1.94 -1.81 (m, 2H), 1.08 (t, J = 7.2 Hz, 3H). MS (ESI) m/z: 599 [M+H]⁺. |
| I-378 | 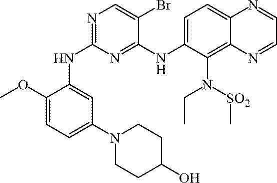 [SMILES: CCN(c1c(Nc2nc(Nc3cc(N4CCC(O)CC4)ccc3OC)ncc2Br)ccc2nccnc12)S(C)(=O)=O] | ¹H NMR (600 MHz, Methanol-d₄) δ 8.93 (d, J = 1.9 Hz, 1H), 8.91 (d, J = 9.6 Hz, 1H), 8.85 (d, J = 1.9 Hz, 1H), 8.42 (s, 1H), 8.35 (s, 1H), 8.09 (d, J = 9.4 Hz, 1H), 7.20 - 7.10 (m, 2H), 4.24 - 4.08 (m, 1H), 3.96 (s, 3H), 3.89 - 3.73 (m, 2H), 3.63 - 3.51 (m, 2H), 3.29 - 3.13 (m, 5H), 2.10 - 1.87 (m, 2H), 1.83 - 1.69 (m, 2H), 1.09 (t, J = 7.2 Hz, 3H). MS (ESI) m/z: 643 [M+H]⁺. |
| I-379 | 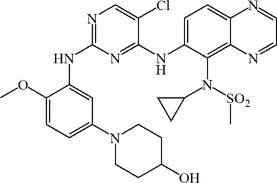 [SMILES: COc1ccc(N2CCC(O)CC2)cc1Nc1ncc(Cl)c(Nc2ccc3nccnc3c2N(C2CC2)S(C)(=O)=O)n1] | ¹H NMR (600 MHz, Methanol-d₄) δ 8.96 -8.91 (m, 2H), 8.81 (d, J= 1.8 Hz, 1H), 8.55 (s, 1H), 8.22 (s, 1H), 8.01 (d, J = 9.4 Hz, 1H), 7.23 (dd, J = 8.8, 2.9 Hz, 1H), 7.14 (d, J = 8.9 Hz, 1H), 3.97 (s, 3H), 3.92 - 3.83 (m, 1H), 3.75 - 3.61 (m, 3H), 3.45 - 3.33 (m, 5H), 2.17 - 1.99 (m, 2H), 1.95 - 1.80 (m, 2H), 1.36 - 1.22 (m, 1H), 1.08 - 0.96 (m, 1H), 0.53 - 0.42 (m, 1H), 0.41 - 0.31 (m, 1H). MS (ESI) m/z: 611 [M+H]⁺. |
| I-380 | 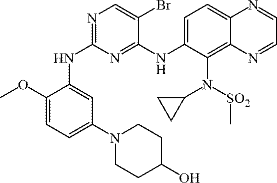 [SMILES: COc1ccc(N2CCC(O)CC2)cc1Nc1ncc(Br)c(Nc2ccc3nccnc3c2N(C2CC2)S(C)(=O)=O)n1] | ¹H NMR (600 MHz, DMSO-d₆) δ 8.99 (d, J = 1.9 Hz, 1H), 8.99 - 8.95 (m, 1H), 8.94 (d, J = 1.9 Hz, 1H), 8.73 - 8.65 (m, 1H), 8.39 (s, 2H), 8.01 (d, J = 9.4 Hz, 1H), 7.94 - 7.67 (m, 1H), 7.16 (d, J = 7.0 Hz, 1H), 3.83 (s, 3H), 3.56 - 3.50 (m, 3H), 3.46 -3.41 (m, 2H), 3.39 (s, 3H), 3.15 - 3.01 (m, 1H), 1.95 - 1.75 (m, 2H), 1.68 - 1.50 (m, 2H), 1.29 - 1.21 (m, 1H), 1.06 - 0.94 (m, 1H), 0.53 - 0.40 (m, 1H), 0.33 - 0.25 (m, 1H). MS (ESI) m/z: 655 [M+H]⁺. |
| I-381 | 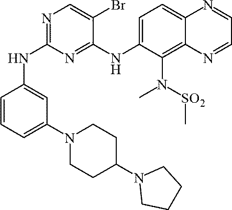 [SMILES: CN(c1c(Nc2nc(Nc3cccc(N4CCC(N5CCCC5)CC4)c3)ncc2Br)ccc2nccnc12)S(C)(=O)=O] | ¹H NMR (600 MHz, Methanol-d₄) δ 9.03 (d, J = 9.4 Hz, 1H), 8.93 (d, J = 1.8 Hz, 1H), 8.83 (d, J = 1.9 Hz, 1H), 8.24 (s, 1H), 8.02 (d, J = 9.4 Hz, 1H), 7.22 (s, 1H), 7.18 - 7.14 (m, 1H), 7.05 (d, J = 7.9 Hz, 1H), 6.77 - 6.70 (m, 1H), 3.77 - 3.72 (m, 2H), 3.65 - 3.59 (m, 2H), 3.47 (s, 3H), 3.24 -3.22 (m, 4H), 3.15 - 3.06 (m, 2H), 2.77 -2.69 (m, 2H), 2.18 - 2.09 (m, 4H), 2.01 -1.95 (m, 2H), 1.80 - 1.72 (m, 2H). MS (ESI) m/z: 652 [M+H]⁺. |
| I-382 | 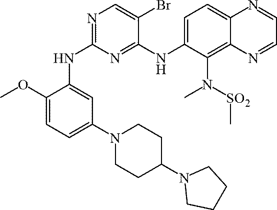 [SMILES: COc1ccc(N2CCC(N3CCCC3)CC2)cc1Nc1ncc(Br)c(Nc2ccc3nccnc3c2N(C)S(C)(=O)=O)n1] | ¹H NMR (600 MHz, DMSO-d₆) δ 9.82 (s, 1H), 8.98 (d, J = 1.9 Hz, 1H), 8.93 (d, J = 1.9 Hz, 1H), 8.90 - 8.83 (m, 1H), 8.60 (s, 1H), 8.45 (s, 1H), 8.36 (s, 1H), 7.97 (d, J = 9.4 Hz, 1H), 7.43 - 7.32 (m, 1H), 7.02 -6.94 (m, 1H), 6.75 (dd, J = 8.9, 2.9 Hz, 1H), 3.75 (s, 3H), 3.54 - 3.47 (m, 4H), 3.43 - 3.37 (m, 7H), 3.24 (s, 3H), 3.07 -3.03 (m, 1H), 2.06 - 1.92 (m, 4H), 1.88 -1.80 (m, 2H), 1.64 - 1.52 (m, 2H). MS (ESI) m/z: 682 [M+H]⁺. |
| I-383 | 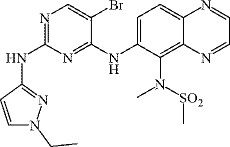 [SMILES: CCn1ccc(Nc2ncc(Br)c(Nc3ccc4nccnc4c3N(C)S(C)(=O)=O)n2)n1] | 1H NMR (600 MHz, DMSO-d₆) δ 10.53 -10.11 (m, 1H), 9.12 - 9.06 (m, 1H), 9.01 (d, J = 1.9 Hz, 1H), 8.97 (d, J = 1.8 Hz, 1H), 8.42 (s, 1H), 8.13 (d, J = 9.3 Hz, 1H), 7.67 - 7.62 (m, 1H), 6.32 - 6.24 (m, 1H), 4.08 (q, J = 7.3 Hz, 2H), 3.41 (s, 3H), 3.25 (s, 3H), 1.38 (t, J = 7.3 Hz, 3H). MS (ESI) m/z: 518 [M+H]⁺. |
| I-384 | 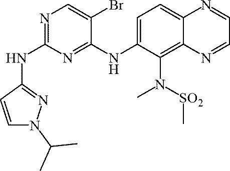 [SMILES: CC(C)n1ccc(Nc2ncc(Br)c(Nc3ccc4nccnc4c3N(C)S(C)(=O)=O)n2)n1] | ¹H NMR (600 MHz, DMSO-d₆) δ 10.24 -10.05 (m, 1H), 9.36 - 9.14 (m, 1H), 9.00 (d, J = 1.9 Hz, 1H), 8.95 (d, J = 1.9 Hz, 1H), 8.84 - 8.78 (m, 1H), 8.39 (s, 1H), 8.09 (d, J = 9.4 Hz, 1H), 7.65 (d, J = 2.4 Hz, 1H), 6.39 - 6.29 (m, 1H), 4.42 (p, J = 6.7 Hz, 1H), 3.41 (s, 3H), 3.26 (s, 3H), 1.41 (d, J = 6.7 Hz, 6H). MS (ESI) m/z: 532 [M+H]⁺. |
| I-385 | 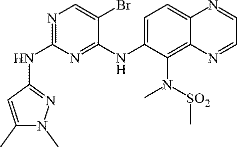 [SMILES: Cc1cc(Nc2ncc(Br)c(Nc3ccc4nccnc4c3N(C)S(C)(=O)=O)n2)nn1C] | ¹H NMR (600 MHz, Chloroform-d) δ 10.06 (s, 1H), 9.80 (s, 1H), 9.23 (d, J = 2.0 Hz, 1H), 9.03 (d, J = 2.0 Hz, 1H), 8.72 (d, J = 9.3 Hz, 1H), 8.37 (d, J = 9.3 Hz, 1H), 8.35 (s, 1H), 6.21 (s, 1H), 3.81 (s, 3H), 3.53 (s, 3H), 3.19 (s, 3H), 2.33 (s, 3H). MS (ESI) m/z: [M+H]⁺. |
| I-386 | 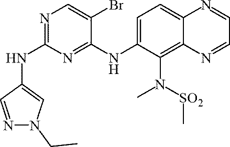 [SMILES: CCn1cc(Nc2ncc(Br)c(Nc3ccc4nccnc4c3N(C)S(C)(=O)=O)n2)cn1] | ¹H NMR (600 MHz, DMSO-d₆) δ 9.45 (s, 1H), 9.33 - 9.17 (m, 1H), 9.00 (d, 1H), 8.98 - 8.93 (m, 1H), 8.61 (s, 1H), 8.33 (s, 1H), 8.15 (d, J = 9.3 Hz, 1H), 7.96 - 7.85 (m, 1H), 7.53 - 7.29 (m, 1H), 4.17 - 3.76 (m, 2H), 3.38 (s, 3H), 3.23 (s, 3H), 1.41 -1.07 (m, 3H). MS (ESI) m/z: 518 [M+H]⁺. |
| I-387 | 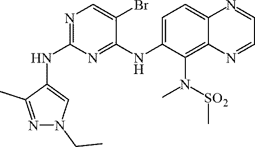 [SMILES: CCn1cc(Nc2ncc(Br)c(Nc3ccc4nccnc4c3N(C)S(C)(=O)=O)n2)c(C)n1] | 1H NMR (600 MHz, DMSO-d₆) δ 9.14 (s, 1H), 9.01 (d, J = 1.8 Hz, 1H), 8.97 (d, J = 1.9 Hz, 1H), 8.71 - 8.55 (m, 1H), 8.44 -8.28 (m, 1H), 8.08 - 7.97 (m, 1H), 7.89 -7.74 (m, 1H), 7.51 (s, 1H), 4.10 - 3.79 (m, 2H), 3.38 (s, 3H), 3.23 (s, 3H), 2.07 (s, 3H), 1.39 - 1.09 (m, 3H). MS (ESI) m/z: 532 [M+H]⁺. |
| I-388 | 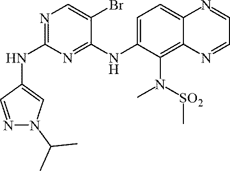 [SMILES: CC(C)n1cc(Nc2ncc(Br)c(Nc3ccc4nccnc4c3N(C)S(C)(=O)=O)n2)cn1] | 1H NMR (600 MHz, DMSO-d₆) δ 9.43 (s, 1H), 9.04 - 8.92 (m, 2H), 8.67 - 8.55 (m, 2H), 8.41 - 8.26 (m, 1H), 8.14 (s, 1H), 7.54 - 7.29 (m, 2H), 4.54 - 4.09 (m, 1H), 3.38 (s, 3H), 3.23 (s, 3H), 1.52 - 1.08 (m, 6H). MS (ESI) m/z: 532 [M+H]⁺. |
| I-389 | 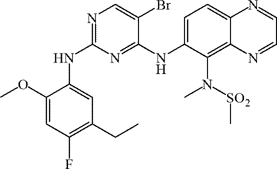 [SMILES: CCc1cc(Nc2ncc(Br)c(Nc3ccc4nccnc4c3N(C)S(C)(=O)=O)n2)c(OC)cc1F] | 1H NMR (600 MHz, DMSO-d₆) δ 8.98 (d, J = 1.9 Hz, 1H), 8.93 (d, J = 1.9 Hz, 1H), 8.83 - 8.73 (m, 1H), 8.65 (s, 1H), 8.57 (s, 1H), 8.33 (s, 1H), 7.95 (d, J = 9.4 Hz, 1H), 7.43 (d, J = 8.3 Hz, 1H), 7.01 (d, J = 11.8 Hz, 1H), 3.78 (s, 3H), 3.38 (s, 3H), 3.23 (s, 3H), 2.43 (q, J = 7.5 Hz, 2H), 0.97 (t, J = 7.6 Hz, 3H). MS (ESI) m/z: 576 [M+H]⁺. |
| I-390 | 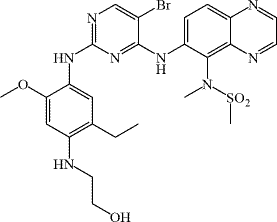 [SMILES: CCc1cc(Nc2ncc(Br)c(Nc3ccc4nccnc4c3N(C)S(C)(=O)=O)n2)c(OC)cc1NCCO] | 1H NMR (600 MHz, Methanol-d₄) δ 8.97 (d, J = 1.8 Hz, 1H), 8.88 (d, J = 1.8 Hz, 1H), 8.72 - 8.62 (m, 1H), 8.23 (s, 1H), 7.97 (d, J = 9.4 Hz, 1H), 7.39 (s, 1H), 6.74 (s, 1H), 4.29 (t, J = 5.1 Hz, 2H), 3.85 (s, 3H), 3.47 (s, 3H), 3.43 (t, J = 5.0 Hz, 2H), 3.19 (s, 3H), 2.51 (q, J = 7.6 Hz, 2H), 0.99 - 0.87 (m, 3H). MS (ESI) m/z: 617 [M+H]⁺. |
| I-391 | 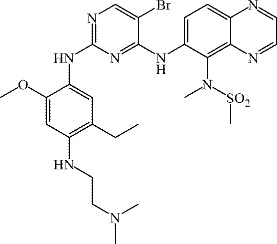 [SMILES: CCc1cc(Nc2ncc(Br)c(Nc3ccc4nccnc4c3N(C)S(C)(=O)=O)n2)c(OC)cc1NCCN(C)C] | ¹H NMR (600 MHz, DMSO-d₆) δ 9.91 -9.69 (m, 1H), 8.99 (d, J = 1.8 Hz, 1H), 8.93 (d, J = 1.8 Hz, 1H), 8.90 - 8.79 (m, 2H), 8.32 (s, 1H), 7.95 - 7.81 (m, 1H), 7.02 (s, 1H), 6.38 (s, 1H), 3.79 (s, 3H), 3.57 (t, J = 6.4 Hz, 2H), 3.38 (s, 3H), 3.34 (t, J = 6.4 Hz, 2H), 3.23 (s, 3H), 2.90 (s, 6H), 2.39 -2.32 (m, 2H), 0.98 (s, 3H). MS (ESI) m/z: 644 [M+H]⁺. |
| I-392 | 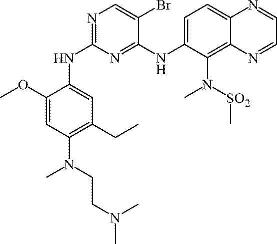 [SMILES: CCc1cc(Nc2ncc(Br)c(Nc3ccc4nccnc4c3N(C)S(C)(=O)=O)n2)c(OC)cc1N(C)CCN(C)C] | 1H NMR (600 MHz, DMSO-d₆) δ 9.75 (s, 1H), 9.20 - 9.12 (m, 2H), 9.02 (d, J = 1.9 Hz, 1H), 8.98 (d, J = 1.8 Hz, 1H), 8.46 (s, 1H), 8.03 (d, J = 9.3 Hz, 1H), 7.37 (s, 1H), 6.93 (s, 1H), 3.83 (s, 3H), 3.38 (s, 3H), 3.25 (s, 4H), 3.22 (s, 3H), 2.83 (s, 6H), 2.61 (s, 3H), 2.45 - 2.29 (m, 2H), 0.84 -0.71 (m, 3H). MS (ESI) m/z: 658 [M+H]⁺. |
| I-393 | 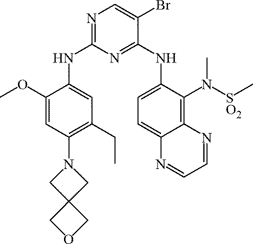 | MS (ESI) m/z: 655 [M+H]⁺. |
| I-394 | 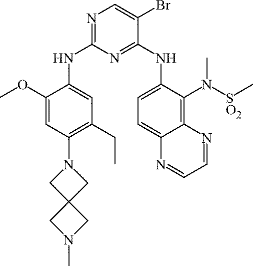 | MS (ESI) m/z: 668 [M+H]⁺. |
| I-395 | 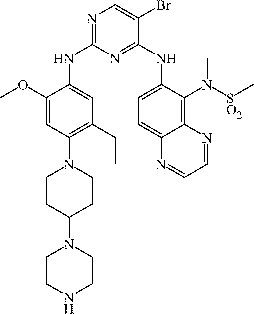 | 1H NMR (600 MHz, DMSO-d₆) δ 8.96 (d, J = 1.9 Hz, 1H), 8.91 (d, J = 1.9 Hz, 1H), 8.70 (s, 1H), 8.45 (s, 1H), 8.31 (s, 1H), 8.11 - 8.07 (m, 1H), 7.94 (d, J = 9.4 Hz, 1H), 7.49 (s, 1H), 6.80 - 6.72 (m, 2H), 5.76 (d, J = 17.5 Hz, 1H), 5.34 (d, J = 11.2 Hz, 1H), 4.37 - 4.33 (m, 1H), 3.77 (s, 3H), 3.32 (s, 3H), 3.18 (s, 3H), 3.01 - 2.93 (m, 2H), 2.74 - 2.61 (m, 6H), 2.45 (s, 4H), 2.36 - 2.30 (m, 2H), 2.29 - 2.23 (m, 1H), 1.86 - 1.78 (m, 2H), 1.60 - 1.50 (m, 2H), 0.83 - 0.76 (m, 3H). MS (ESI) m/z: 725 [M+H]⁺. |
| I-396 | 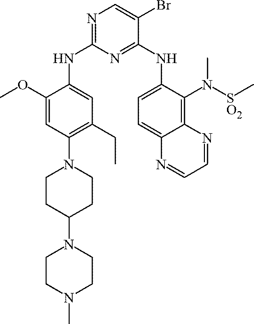 | 1H NMR (600 MHz, DMSO-d₆) δ 9.00 (d, J = 1.9 Hz, 1H), 8.95 (d, J = 1.8 Hz, 1H), 8.81 - 8.73 (m, 2H), 8.66 (s, 1H), 8.37 (s, 1H), 7.98 (d, J = 9.3 Hz, 1H), 7.47 (s, 1H), 6.79 (s, 1H), 3.80 (s, 3H), 3.72 - 3.62 (m, 4H), 3.60 - 3.50 (m, 1H), 3.38 (s, 3H), 3.34 - 3.26 (m, 2H), 3.24 (s, 3H), 3.15 (d, J = 8.4 Hz, 4H), 3.10 - 3.00 (m, 2H), 2.82 (s, 3H), 2.49 - 2.37 (m, 4H), 2.08 - 1.92 (m, 2H), 0.95 - 0.83 (m, 3H). MS (ESI) m/z: 739 [M+H]⁺. |
| I-397 | 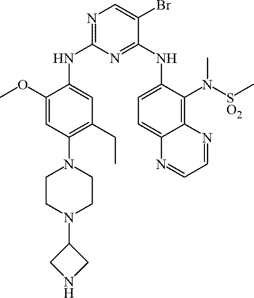 | MS (ESI) m/z: 697 [M+H]⁺. |
| I-398 | 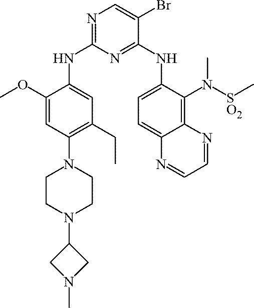 | 1H NMR (600 MHz, DMSO-d₆) δ 9.00 (d, J = 1.9 Hz, 1H), 8.95 (d, J = 1.8 Hz, 1H), 8.83 (s, 1H), 8.74 - 8.69 (m, 2H), 8.37 (s, 1H), 7.96 (d, J = 9.2 Hz, 1H), 7.41 (s, 1H), 6.81 (s, 1H), 4.58 - 4.39 (m, 2H), 4.30 -4.06 (m, 2H), 3.80 (s, 3H), 3.37 (s, 3H), 3.23 (s, 3H), 3.17 - 2.98 (m, 9H), 2.93 (s, 3H), 2.45 - 2.34 (m, 2H), 0.91 - 0.80 (m, 3H). MS (ESI) m/z: 711 [M+H]⁺. |
| I-399 | 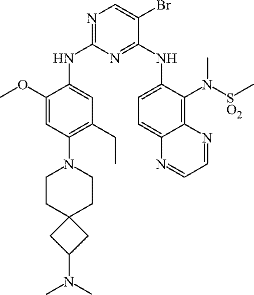 | 1H NMR (600 MHz, DMSO-d₆) δ 9.00 (d, J = 2.0 Hz, 1H), 8.99 - 8.94 (m, 2H), 8.90 (s, 1H), 8.75 - 8.57 (m, 1H), 8.40 (s, 1H), 7.99 (d, J = 9.3 Hz, 1H), 7.38 (s, 1H), 6.85 (s, 1H), 3.79 (s, 3H), 3.77 - 3.68 (m, 1H), 3.36 (s, 3H), 3.22 (s, 3H), 2.93 - 2.81 (m, 4H), 2.72 (s, 3H), 2.71 (s, 3H), 2.44 - 2.35 (m, 2H), 2.32 - 2.20 (m, 2H), 2.09 - 1.96 (m, 2H), 1.82 - 1.68 (m, 4H), 0.84 (s, 3H). MS (ESI) m/z: 724 [M+H]⁺. |
| I-400 | 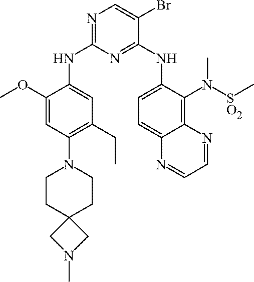 | MS (ESI) m/z: 696 [M+H]⁺. |
| I-401 | 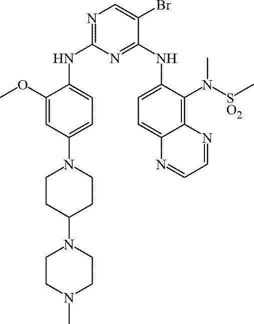 | ¹H NMR (600 MHz, DMSO-d₆) δ 9.99 (s, 1H), 9.13 (s, 1H), 9.02 (d, J = 1.9 Hz, 1H), 8.95 (d, J = 1.9 Hz, 1H), 8.85 (s, 1H), 8.71 - 8.63 (m, 1H), 8.36 (s, 1H), 7.98 (d, J = 9.4 Hz, 1H), 7.41 (d, J = 8.7 Hz, 1H), 6.85 - 6.73 (m, 1H), 6.56 (d, J = 8.8 Hz, 1H), 3.90 - 3.86 (m, 2H), 3.80 (s, 3H), 3.51 (d, J = 129.0 Hz, 9H), 3.05 (s, 3H), 2.90 (s, 3H), 2.88 - 2.80 (m, 2H), 2.22 - 2.14 (m, 2H), 1.82 - 1.72 (m, 2H). MS (ESI) m/z: 697 [M+H]⁺. |
| I-402 | 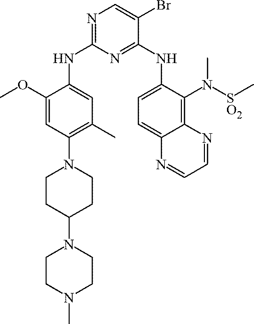 | 1H NMR (600 MHz, DMSO-d₆) δ 9.98 (s, 1H), 9.12 (s, 1H), 9.03 (d, J = 1.8 Hz, 1H), 8.96 (d, J = 1.9 Hz, 1H), 8.67 (s, 1H), 8.64 - 8.57 (m, 1H), 8.37 (s, 1H), 7.94 (d, J = 9.3 Hz, 1H), 7.36 (s, 1H), 6.75 (s, 1H), 3.79 (s, 3H), 3.73 - 3.28 (m, 10H), 3.22 -3.19 (m, 1H), 3.18 (s, 3H), 3.05 (s, 3H), 2.91 (s, 3H), 2.77 - 2.70 (m, 2H), 2.20 -2.11 (m, 2H), 1.86 - 1.76 (m, 2H). MS (ESI) m/z: 711 [M+H]⁺. |
| I-403 | 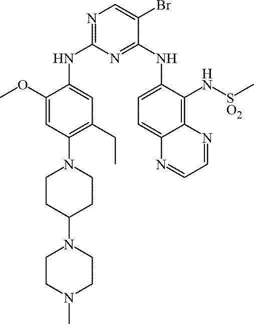 | 1H NMR (600 MHz, DMSO-d₆) δ 10.00 (s, 1H), 9.17 (s, 1H), 9.03 (d, J = 1.8 Hz, 1H), 8.97 (d, J = 1.9 Hz, 1H), 8.79 (s, 1H), 8.62 - 8.50 (m, 1H), 8.40 (s, 1H), 7.93 (d, J = 9.4 Hz, 1H), 7.39 (s, 1H), 6.80 (s, 1H), 3.79 (s, 3H), 3.74 - 3.21 (m, 9H), 3.15 -3.09 (m, 2H), 3.05 (s, 3H), 2.92 (s, 3H), 2.84 - 2.74 (m, 2H), 2.42 - 2.35 (m, 2H), 2.20 - 2.13 (m, 2H), 1.87 - 1.77 (m, 2H), 0.87 - 0.73 (m, 3H). MS (ESI) m/z: 725 [M+H]⁺. |
| I-404 | 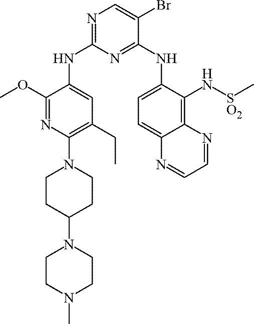 | 1H NMR (600 MHz, DMSO-d₆) δ 9.97 (s, 1H), 9.03 (s, 1H), 9.02 (d, J = 1.9 Hz, 1H), 8.95 (d, J = 1.9 Hz, 1H), 8.64 (s, 1H), 8.60 - 8.51 (m, 1H), 8.35 (s, 1H), 7.93 - 7.87 (m, 1H), 7.67 (s, 1H), 3.85 (s, 3H), 3.79 -3.57 (m, 8H), 3.49 - 3.40 (m, 2H), 3.32 -3.20 (m, 1H), 3.04 (s, 3H), 2.90 (s, 3H), 2.88 - 2.82 (m, 2H), 2.43 - 2.33 (m, 2H), 2.19 - 2.07 (m, 2H), 1.85 - 1.74 (m, 2H), 0.91 - 0.74 (m, 3H). MS (ESI) m/z: 726 [M+H]⁺. |
| I-405 | 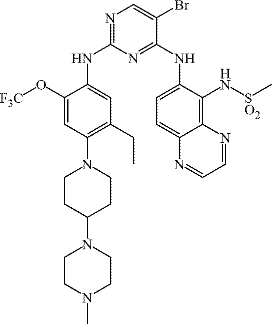 [SMILES: CCc1cc(Nc2ncc(Br)c(Nc3ccc4nccnc4c3NS(C)(=O)=O)n2)c(OC(F)(F)F)cc1N1CCC(N2CCN(C)CC2)CC1] | 1H NMR (600 MHz, DMSO-d₆) δ 9.95 (s, 1H), 9.11 (s, 1H), 9.01 (d, J = 1.8 Hz, 1H), 8.94 (s, 1H), 8.93 (d, J = 1.8 Hz, 1H), 8.64 - 8.55 (m, 1H), 8.33 (s, 1H), 7.82 (d, J = 9.2 Hz, 1H), 7.41 (s, 1H), 7.08 (s, 1H), 3.85 - 3.23 (m, 9H), 3.15 - 3.09 (m, 2H), 3.03 (s, 3H), 2.90 (s, 3H), 2.79 - 2.69 (m, 2H), 2.56 - 2.52 (m, 2H), 2.19 - 2.12 (m, 2H), 1.85 - 1.75 (m, 2H), 0.98 (t, J = 7.5 Hz, 3H). MS (ESI) m/z: 779 [M+H]⁺. |
| I-406 | 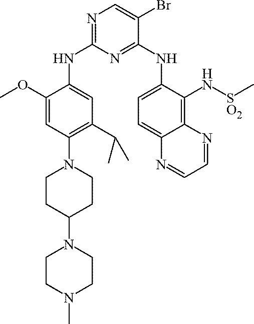 | 1H NMR (600 MHz, DMSO-d₆) δ 10.00 (s, 1H), 9.08 (s, 1H), 9.02 (d, J = 1.9 Hz, 1H), 8.94 (d, J = 1.9 Hz, 1H), 8.83 (s, 1H), 8.67 - 8.51 (m, 1H), 8.37 (s, 1H), 7.95 - 7.79 (m, 1H), 7.40 - 7.29 (m, 1H), 6.85 (s, 1H), 3.77 (s, 3H), 3.72 - 3.48 (m, 8H), 3.47 -3.36 (m, 1H), 3.27 - 3.20 (m, 1H), 3.10 -3.06 (m, 2H), 3.05 (s, 3H), 2.92 (s, 3H), 2.89 - 2.80 (m, 2H), 2.24 - 2.11 (m, 2H), 1.92 - 1.78 (m, 2H), 1.00 - 0.90 (m, 6H). MS (ESI) m/z: 739 [M+H]⁺. |
| I-407 | 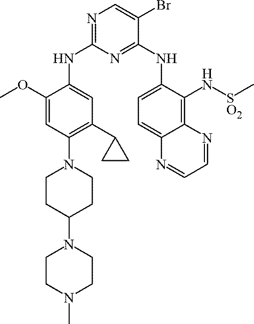 | ¹H NMR (600 MHz, DMSO-d₆) δ 10.00 (s, 1H), 9.13 - 9.06 (m, 1H), 9.02 (d, J = 1.9 Hz, 1H), 8.94 (d, J = 2.0 Hz, 1H), 8.87 -8.81 (m, 1H), 8.63 - 8.51 (m, 1H), 8.35 (s, 1H), 7.91 - 7.82 (m, 1H), 6.98 - 6.87 (m, 1H), 6.75 (s, 1H), 3.78 (s, 3H), 3.73 - 3.47 (m, 8H), 3.46 - 3.38 (m, 2H), 3.26 - 3.21 (m, 1H), 3.04 (s, 3H), 2.91 (s, 3H), 2.83 -2.73 (m, 2H), 2.25 - 2.14 (m, 2H), 2.09 -2.01 (m, 1H), 1.95 - 1.80 (m, 2H), 0.73 -0.62 (m, 2H), 0.41 - 0.31 (m, 2H). MS (ESI) m/z: 737 [M+H]⁺. |
| I-408 | 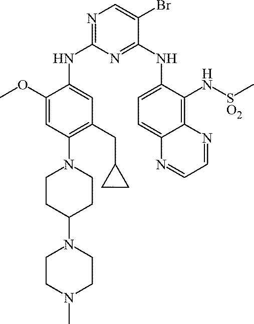 | 1H NMR (600 MHz, DMSO-d₆) δ 9.98 (s, 1H), 9.06 (s, 1H), 9.02 (d, J = 1.9 Hz, 1H), 8.95 (d, J = 1.9 Hz, 1H), 8.64 (s, 1H), 8.60 - 8.53 (m, 1H), 8.35 (s, 1H), 7.89 (d, J = 9.2 Hz, 1H), 7.53 (s, 1H), 6.80 (s, 1H), 3.78 (s, 3H), 3.70 - 3.40 (m, 8H), 3.38 -3.30 (m, 1H), 3.12 - 3.06 (m, 2H), 3.03 (s, 3H), 2.90 (s, 3H), 2.81 - 2.72 (m, 2H), 2.34 - 2.25 (m, 2H), 2.18 - 2.10 (m, 2H), 1.82 - 1.69 (m, 2H), 0.68 - 0.52 (m, 1H), 0.27 - 0.14 (m, 2H), -0.09 - -0.19 (m, 2H). MS (ESI) m/z: 751 [M+H]⁺. |
| I-409 | 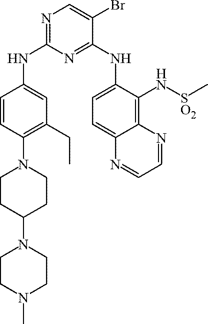 [SMILES: CCc1cc(Nc2ncc(Br)c(Nc3ccc4nccnc4c3NS(C)(=O)=O)n2)ccc1N1CCC(N2CCN(C)CC2)CC1] | 1H NMR (600 MHz, DMSO-d₆) δ 9.97 (s, 1H), 9.48 (s, 1H), 9.03 (d, J = 1.9 Hz, 1H), 8.99 - 8.94 (m, 2H), 8.81 - 8.67 (m, 1H), 8.36 (s, 1H), 8.05 (d, J = 9.3 Hz, 1H), 7.44 - 7.37 (m, 2H), 6.97 (d, J = 8.5 Hz, 1H), 3.72 - 3.40 (m, 8H), 3.33 - 3.21 (m, 1H), 3.08 - 3.00 (m, 5H), 2.87 (s, 3H), 2.74 -2.63 (m, 2H), 2.49 - 2.47 (m, 2H), 2.16 -2.05 (m, 2H), 1.82 - 1.69 (m, 2H), 0.98 -0.89 (m, 3H). MS (ESI) m/z: 695 [M+H]⁺. |
| I-410 | 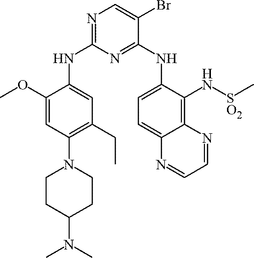 | 1H NMR (600 MHz, DMSO-d₆) δ 10.00 (s, 1H), 9.18 (s, 1H), 9.03 (d, J = 1.9 Hz, 1H), 8.97 (d, J = 1.9 Hz, 1H), 8.79 (s, 1H), 8.60 - 8.47 (m, 1H), 8.39 (s, 1H), 7.92 (d, J = 9.3 Hz, 1H), 7.39 (s, 1H), 6.80 (s, 1H), 3.79 (s, 3H), 3.32 - 3.25 (m, 1H), 3.13 -3.07 (m, 2H), 3.05 (s, 3H), 2.84 (s, 3H), 2.83 (s, 3H), 2.80 - 2.73 (m, 2H), 2.39 (q, J = 7.5 Hz, 2H), 2.17 - 2.05 (m, 2H), 1.85 - 1.75 (m, 2H), 0.84 - 0.77 (m, 3H). MS (ESI) m/z: 670 [M+H]⁺. |
| I-411 | 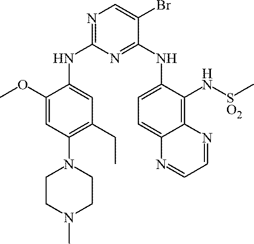 | 1H NMR (600 MHz, DMSO-d₆) δ 9.98 (s, 1H), 9.07 (s, 1H), 9.05 - 9.00 (m, 1H), 8.96 (d, J = 1.9 Hz, 1H), 8.56 (s, 1H), 8.37 (s, 1H), 7.94 (d, J = 9.3 Hz, 1H), 7.47 (s, 1H), 6.85 - 6.63 (m, 2H), 3.81 (s, 3H), 3.57 - 3.49 (m, 2H), 3.27 - 3.19 (m, 2H), 3.14 - 3.09 (m, 2H), 3.06 - 2.99 (m, 5H), 2.91 (s, 3H), 2.44 - 2.37 (m, 2H), 0.92 -0.73 (m, 3H). MS (ESI) m/z: 642 [M+H]⁺. |
| I-412 | 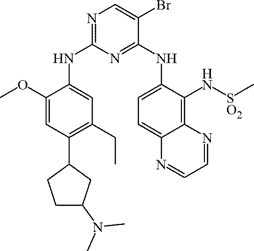 | 1H NMR (600 MHz, DMSO-d₆) δ 9.99 (s, 1H), 9.10 (s, 1H), 9.03 (d, J = 1.9 Hz, 1H), 8.96 (d, J = 1.9 Hz, 1H), 8.70 (s, 1H), 8.60 - 8.53 (m, 1H), 8.36 (s, 1H), 7.91 (d, J = 9.2 Hz, 1H), 7.34 (s, 1H), 6.78 (s, 1H), 3.79 (s, 3H), 3.36 - 3.30 (m, 2H), 3.23 -3.18 (m, 2H), 3.12 - 3.06 (m, 1H), 3.04 (s, 3H), 2.88 (s, 3H), 2.87 (s, 3H), 2.45 - 2.38 (m, 2H), 2.37 - 2.30 (m, 1H), 2.22 - 2.08 (m, 1H), 0.89 - 0.78 (m, 3H). MS (ESI) m/z: 656 [M+H]⁺. |
| I-413 | 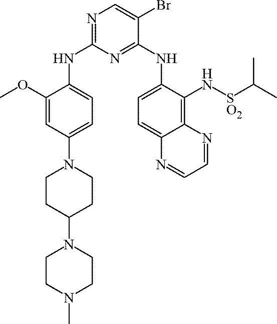 [SMILES: COc1cc(N2CCC(N3CCN(C)CC3)CC2)ccc1Nc1ncc(Br)c(Nc2ccc3nccnc3c2NS(=O)(=O)C(C)C)n1] | ¹H NMR (600 MHz, DMSO-d₆) δ 9.85 (s, 1H), 9.23 (s, 1H), 9.01 (d, J = 1.7 Hz, 1H), 8.95 (d, J = 1.7 Hz, 1H), 8.86 - 8.78 (m, 1H), 8.56 (d, J = 9.2 Hz, 1H), 8.36 (s, 1H), 7.98 (d, J = 9.2 Hz, 1H), 7.39 (d, J = 8.6 Hz, 1H), 6.78 (s, 1H), 6.52 (d, J = 8.6 Hz, 1H), 3.90 - 3.83 (m, 2H), 3.80 (s, 3H), 3.73 - 3.45 (m, 8H), 3.42 - 3.33 (m, 1H), 3.29 (hept, J = 6.8 Hz, 1H), 2.89 (s, 3H), 2.87 - 2.78 (m, 2H), 2.20 - 2.11 (m, 2H), 1.80 - 1.70 (m, 2H), 1.38 (d, J = 6.8 Hz, 6H). MS (ESI) m/z: 725 [M+H]⁺. |
| I-414 | 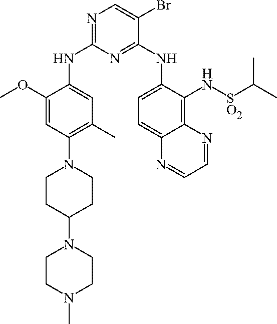 [SMILES: COc1cc(N2CCC(N3CCN(C)CC3)CC2)c(C)cc1Nc1ncc(Br)c(Nc2ccc3nccnc3c2NS(=O)(=O)C(C)C)n1] | ¹H NMR (600 MHz, DMSO-d₆) δ 9.84 (s, 1H), 9.17 (s, 1H), 9.01 (d, J = 1.9 Hz, 1H), 8.94 (d, J = 1.9 Hz, 1H), 8.55 - 8.46 (m, 2H), 8.36 (s, 1H), 7.93 (d, J = 9.3 Hz, 1H), 7.36 (s, 1H), 6.73 (s, 1H), 3.78 (s, 3H), 3.75 - 3.44 (m, 8H), 3.38 - 3.24 (m, 2H), 3.16 - 3.12 (m, 2H), 2.89 (s, 3H), 2.75 -2.67 (m, 2H), 2.16 - 2.09 (m, 2H), 1.95 (s, 3H), 1.84 - 1.73 (m, 2H), 1.38 (d, J = 6.8 Hz, 6H). MS (ESI) m/z: 739 [M+H]⁺. |
| I-415 | 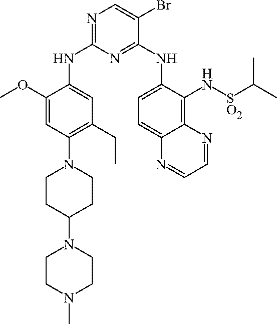 [SMILES: CCc1cc(Nc2ncc(Br)c(Nc3ccc4nccnc4c3NS(=O)(=O)C(C)C)n2)c(OC)cc1N1CCC(N2CCN(C)CC2)CC1] | ¹H NMR (600 MHz, DMSO-d₆) δ 9.87 (s, 1H), 9.27 (s, 1H), 9.00 (d, J = 1.9 Hz, 1H), 8.95 (d, J = 1.9 Hz, 1H), 8.71 (s, 1H), 8.46 - 8.40 (m, 1H), 8.38 (s, 1H), 7.91 (d, J = 9.2 Hz, 1H), 7.38 (s, 1H), 6.77 (s, 1H), 3.77 (s, 3H), 3.60 (d, J = 116.1 Hz, 8H), 3.42 - 3.34 (m, 1H), 3.28 (hept, J = 6.7 Hz, 1H), 3.09 - 3.03 (m, 2H), 2.90 (s, 3H), 2.80 - 2.71 (m, 2H), 2.31 (s, 2H), 2.17 -2.10 (m, 2H), 1.84 - 1.74 (m, 2H), 1.36 (d, J = 6.7 Hz, 6H), 0.79 - 0.65 (m, 3H). MS (ESI) m/z: 753 [M+H]⁺. |
| II-1-1 | 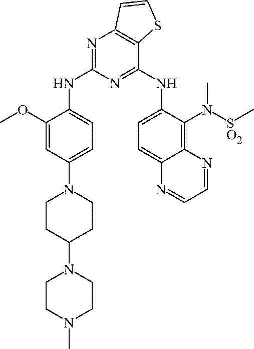 | ¹H NMR (600 MHz, Methanol-d₄) δ 9.01 (d, J = 1.7 Hz, 1H), 8.98 (d, J = 1.7 Hz, 1H), 8.35 - 8.24 (m, 2H), 8.15 (d, J = 9.2 Hz, 1H), 7.40 - 7.26 (m, 2H), 6.85 (d, J = 2.4 Hz, 1H), 6.62 - 6.50 (m, 1H), 3.89 -3.81 (m, 5H), 3.60 - 3.41 (m, 11H), 3.28 -3.21 (m, 1H), 3.12 (s, 3H), 3.08 - 3.01 (m, 2H), 2.96 (s, 3H), 2.32 - 2.19 (m, 2H), 2.04 - 1.83 (m, 2H). MS (ESI) m/z: 689 [M+H]⁺. |
| II-1-2 | 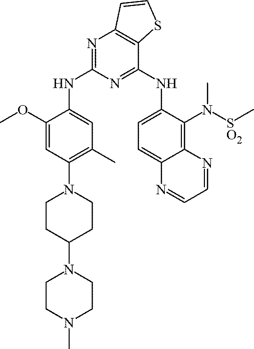 | 1H NMR (600 MHz, MeOD) δ 8.98 (d, J = 1.8 Hz, 1H), 8.95 (d, J = 1.8 Hz, 1H), 8.25 (d, J = 5.5 Hz, 1H), 8.21 - 8.15 (m, 1H), 8.10 (d, J = 9.2 Hz, 1H), 7.29 (d, J = 5.5 Hz, 1H), 7.16 (s, 1H), 6.72 (s, 1H), 3.79 (s, 3H), 3.54 (s, 8H), 3.41 (s, 3H), 3.26 - 3.22 (m, 1H), 3.22 - 3.15 (m, 2H), 3.09 (s, 3H), 2.93 (s, 3H), 2.81 - 2.73 (m, 2H), 2.24 -2.12 (m, 2H), 1.97 - 1.89 (m, 2H), 1.78 (br s, 3H). MS (ESI) m/z: 703 [M+H]⁺. |
| II-1-3 | 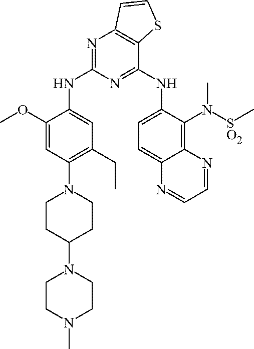 | 1H NMR (600 MHz, MeOD) δ 9.00 - 8.97 (m, 1H), 8.96 - 8.92 (m, 1H), 8.25 (d, J = 5.6 Hz, 1H), 8.21 - 8.13 (m, 1H), 8.08 (d, J = 8.9 Hz, 1H), 7.30 (d, J = 5.5 Hz, 1H), 7.23 (s, 1H), 6.76 (s, 1H), 3.79 (s, 3H), 3.64 - 3.47 (m, 8H), 3.42 (s, 3H), 3.26 -3.19 (m, 1H), 3.10 (s, 2H), 3.07 (s, 3H), 2.92 (s, 3H), 2.87 - 2.72 (m, 2H), 2.49 -2.20 (m, 2H), 2.20 - 2.12 (m, 2H), 1.95 -1.81 (m, 2H), 1.02 - 0.50 (m, 3H). MS (ESI) m/z: 717 [M+H]⁺. |
| II-1-4 | 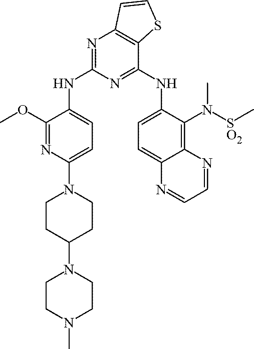 | ¹H NMR (600 MHz, MeOD) δ 8.98 (d, J = 1.8 Hz, 1H), 8.94 (d, J = 1.8 Hz, 1H), 8.38 - 8.20 (m, 2H), 8.16 - 8.01 (m, 1H), 7.45 -7.34 (m, 1H), 7.33 - 7.22 (m, 1H), 6.49 -5.96 (m, 1H), 4.56 - 4.40 (m, 2H), 3.83 (s, 3H), 3.52 - 3.43 (m, 8H), 3.34 - 3.31 (m, 1H), 3.28 (s, 3H), 3.09 (s, 3H), 2.90 (s, 3H), 2.90 - 2.84 (m, 2H), 2.17 - 2.08 (m, 2H), 1.80 - 1.60 (m, 2H). MS (ESI) m/z: 690 [M+H]⁺. |
| II-1-5 | 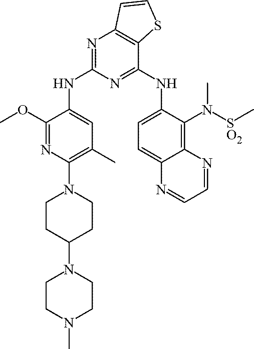 | ¹H NMR (600 MHz, DMSO-d₆) δ 10.88 (br s, 1H), 9.60 (br s, 1H), 9.06 (s, 2H), 8.35 (d, J = 5.3 Hz, 1H), 8.22 - 8.05 (m, 2H), 7.41 - 7.36 (m, 1H), 7.34 (d, J = 5.4 Hz, 1H), 3.82 (s, 3H), 3.75 - 3.62 (m, 2H), 3.53 - 3.20 (m, 8H), 3.20 - 3.03 (m, 6H), 2.82 (s, 3H), 2.78 - 2.68 (m, 2H), 1.92 (s, 3H), 1.86 - 1.25 (m, 5H). MS (ESI) m/z: 704 [M+H]⁺. |
| II-1-6 | 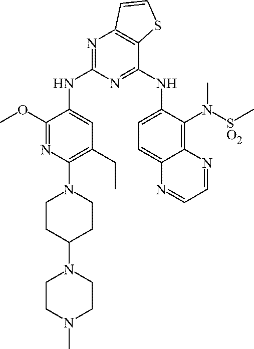 | ¹H NMR (600 MHz, MeOD) δ 8.96 (d, J = 1.9 Hz, 1H), 8.95 - 8.92 (m, 1H), 8.24 (d, J = 5.5 Hz, 1H), 8.12 - 8.05 (m, 1H), 8.05 -8.01 (m, 1H), 7.48 (s, 1H), 7.29 (d, J = 5.4 Hz, 1H), 3.85 (s, 3H), 3.71 - 3.57 (m, 8H), 3.48 - 3.39 (m, 5H), 3.38 - 3.33 (m, 1H), 3.05 (s, 3H), 2.94 (s, 3H), 2.91 - 2.79 (m, 2H), 2.29 - 2.15 (m, 2H), 2.18 - 2.11 (m, 2H), 1.94 - 1.82 (m, 2H), 1.04 - 0.30 (m, 3H). MS (ESI) m/z: 718 [M+H]⁺. |
| II-1-7 | 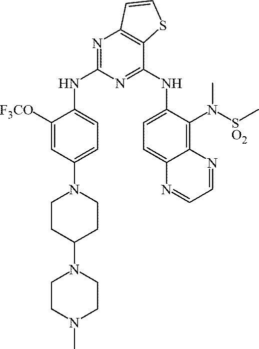 [SMILES: CN1CCN(C2CCN(c3ccc(Nc4nc(Nc5ccc6nccnc6c5N(C)S(C)(=O)=O)c5sccc5n4)c(OC(F)(F)F)c3)CC2)CC1] | ¹H NMR (600 MHz, Methanol-d₄) δ 8.99 (d, J = 8.7 Hz, 2H), 8.28 (d, J = 5.5 Hz, 1H), 8.16 (d, J = 8.8 Hz, 1H), 8.07 (d, J = 9.3 Hz, 1H), 7.35 (d, J = 5.5 Hz, 1H), 7.29 (d, J = 8.9 Hz, 1H), 6.88 (s, 1H), 6.77 (s, 1H), 3.93 - 3.80 (m, 2H), 3.77 - 3.49 (m, 8H), 3.41 (s, 3H), 3.38 - 3.33 (m, 1H), 3.11 (s, 3H), 2.97 (s, 3H), 2.89 - 2.77 (m, 2H), 2.29 - 2.20 (m, 2H), 1.97 - 1.81 (m, 2H). MS (ESI) m/z: 743 [M+H]⁺. |
| II-1-8 | 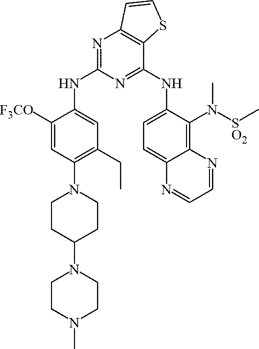 [SMILES: CCc1cc(Nc2nc(Nc3ccc4nccnc4c3N(C)S(C)(=O)=O)c3sccc3n2)c(OC(F)(F)F)cc1N1CCC(N2CCN(C)CC2)CC1] | ¹H NMR (600 MHz, MeOD) δ 8.94 - 8.92 (m, 1H), 8.92 - 8.91 (m, 1H), 8.25 (d, J = 5.6 Hz, 1H), 8.01 - 7.85 (m, 2H), 7.31 (d, J = 5.5 Hz, 1H), 7.24 (s, 1H), 6.94 (s, 1H), 3.73 - 3.54 (m, 8H), 3.35 (s, 3H), 3.33 -3.30 (m, 1H), 3.03 (s, 3H), 3.02 - 2.98 (m, 2H), 2.93 (s, 3H), 2.72 - 2.58 (m, 2H), 2.38 - 2.23 (m, 1H), 2.23 - 2.11 (m, 2H), 2.10 - 1.92 (m, 1H), 1.91 - 1.79 (m, 2H), 0.83 - 0.63 (m, 3H). MS (ESI) m/z: 771 [M+H]⁺. |
| II-1-9 | 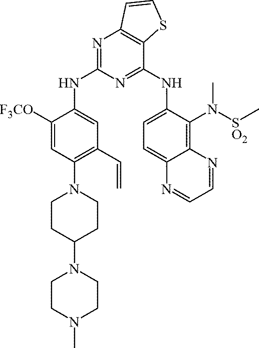 [SMILES: C=Cc1cc(Nc2nc(Nc3ccc4nccnc4c3N(C)S(C)(=O)=O)c3sccc3n2)c(OC(F)(F)F)cc1N1CCC(N2CCN(C)CC2)CC1] | ¹H NMR (600 MHz, MeOD) δ 8.99 (d, J = 1.8 Hz, 1H), 8.97 (d, J = 1.9 Hz, 1H), 8.33 (d, J = 5.6 Hz, 1H), 8.01 - 7.95 (m, 1H), 7.94 - 7.86 (m, 1H), 7.54 (s, 1H), 7.38 (d, J = 5.5 Hz, 1H), 6.96 (s, 1H), 6.62 - 6.49 (m, 1H), 5.16 (s, 1H), 4.89 (s, 1H), 3.65 -3.51 (m, 8H), 3.43 (s, 3H), 3.28 - 3.19 (m, 3H), 3.11 (s, 3H), 2.96 (s, 3H), 2.79 - 2.62 (m, 2H), 2.26 - 2.14 (m, 2H), 1.97 - 1.87 (m, 2H). MS (ESI) m/z: 769 [M+H]⁺. |
| II-1-10 | 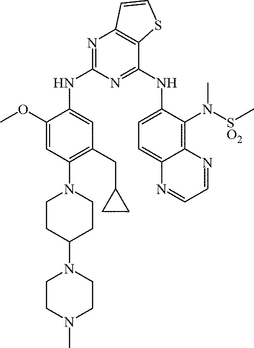 | ¹H NMR (600 MHz, MeOD) δ 8.96 (d, J = 1.8 Hz, 1H), 8.93 (d, J = 1.9 Hz, 1H), 8.22 (d, J = 5.4 Hz, 1H), 8.18 - 8.08 (m, 1H), 8.07 - 7.94 (m, 1H), 7.37 (s, 1H), 7.28 (d, J = 5.5 Hz, 1H), 6.74 (s, 1H), 3.77 (s, 3H), 3.65 - 3.47 (m, 8H), 3.41 (s, 3H), 3.24 -3.20 (m, 1H), 3.10 - 3.03 (m, 5H), 2.91 (s, 3H), 2.82 - 2.69 (m, 2H), 2.16 (dt, J = 9.5, 3.3 Hz, 2H), 1.92 - 1.77 (m, 2H), 0.62 -0.31 (m, 1H), 0.27 - 0.06 (m, 2H), -0.08 --0.39 (m, 2H). MS (ESI) m/z: 742 [M+H]⁺. |
| II-1-11 | 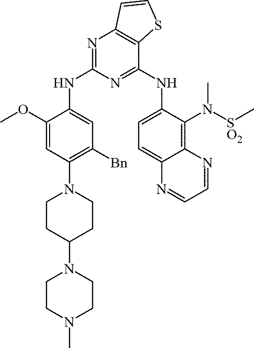 | ¹H NMR (600 MHz, MeOD) δ 8.94 - 8.89 (m, 1H), 8.89 - 8.80 (m, 1H), 8.28 (d, J = 5.6 Hz, 1H), 8.24 - 8.12 (m, 1H), 8.10 -8.03 (m, 1H), 7.43 - 7.26 (m, 2H), 7.15 -6.98 (m, 3H), 6.95 - 6.73 (m, 3H), 3.82 (s, 3H), 3.79 - 3.48 (m, 10H), 3.41 (s, 3H), 3.37 - 3.34 (m, 1H), 3.06 (s, 3H), 2.99 (s, 3H), 2.96 - 2.80 (m, 2H), 2.78 - 2.61 (m, 2H), 2.18 - 2.02 (m, 2H), 1.87 - 1.71 (m, 2H). MS (ESI) m/z: 779 [M+H]⁺. |
| II-1-12 | 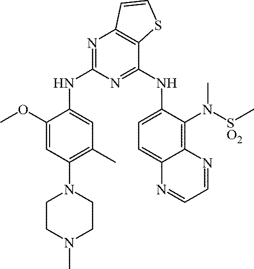 | ¹H NMR (600 MHz, MeOD) δ 9.03 (d, J = 1.8 Hz, 1H), 8.99 (d, J = 1.9 Hz, 1H), 8.29 (d, J = 5.5 Hz, 1H), 8.26 - 8.20 (m, 1H), 8.15 - 8.09 (m, 1H), 7.35 (d, J = 5.5 Hz, 1H), 7.26 (s, 1H), 6.76 (s, 1H), 3.85 (s, 3H), 3.62 (t, J = 11.4 Hz, 2H), 3.48 (s, 3H), 3.34 - 3.28 (m, 2H), 3.27 - 3.18 (m, 2H), 3.14 (s, 3H), 3.13 - 3.06 (m, 2H), 3.01 (s, 3H), 1.83 (br s, 3H). MS (ESI) m/z: 620 [M+H]⁺. |
| II-1-13 | 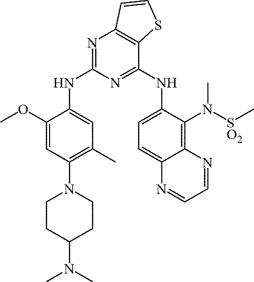 | ¹H NMR (600 MHz, MeOD) δ 9.01 (d, J = 1.8 Hz, 1H), 8.98 (d, J = 1.9 Hz, 1H), 8.28 (d, J = 5.5 Hz, 1H), 8.25 - 8.20 (m, 1H), 8.16 - 8.10 (m, 1H), 7.32 (d, J = 5.1 Hz, 1H), 7.19 (s, 1H), 6.73 (s, 1H), 3.82 (s, 3H), 3.46 (s, 3H), 3.24 - 3.17 (m, 2H), 3.12 (s, 3H), 2.94 (s, 6H), 2.84 - 2.69 (m, 3H), 2.23 - 2.16 (m, 2H), 1.97 - 1.72 (m, 5H). MS (ESI) m/z: 648 [M+H]⁺. |
| II-1-14 | 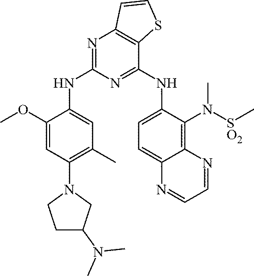 | ¹H NMR (600 MHz, MeOD) δ 9.01 (d, J = 1.8 Hz, 1H), 8.97 (d, J = 1.9 Hz, 1H), 8.35 - 8.26 (m, 1H), 8.26 - 8.17 (m, 1H), 8.16 -8.10 (m, 1H), 7.32 (d, J = 5.4 Hz, 1H), 7.20 - 7.12 (m, 1H), 6.69 (s, 1H), 4.04 - 3.98 (m, 1H), 3.82 (s, 3H), 3.48 (s, 3H), 3.45 -3.37 (m, 4H), 3.18 - 3.14 (m, 1H), 3.12 (s, 3H), 2.99 (s, 3H), 2.98 (s, 3H), 2.55 - 2.45 (m, 1H), 2.25 - 2.18 (m, 1H), 1.73 (br s, 3H). MS (ESI) m/z: 634 [M+H]⁺. |
| II-1-15 | 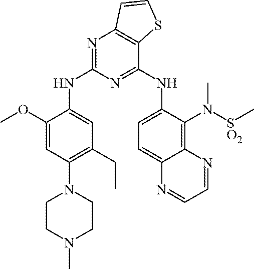 | ¹H NMR (600 MHz, MeOD) δ 9.01 (d, J = 1.8 Hz, 1H), 8.97 (d, J = 1.8 Hz, 1H), 8.29 (d, J = 5.5 Hz, 1H), 8.24 - 8.16 (m, 1H), 8.12 - 8.05 (m, 1H), 7.36 - 7.27 (m, 2H), 6.80 (s, 1H), 3.84 (s, 3H), 3.59 (t, J = 10.1 Hz, 2H), 3.47 (s, 3H), 3.30 - 3.25 (m, 2H), 3.17 - 3.07 (m, 7H), 2.98 (s, 3H), 2.46 -2.25 (m, 1H), 2.18 - 1.92 (m, 1H), 0.72 (br s, 3H). MS (ESI) m/z: 634 [M+H]⁺. |
| II-1-16 | 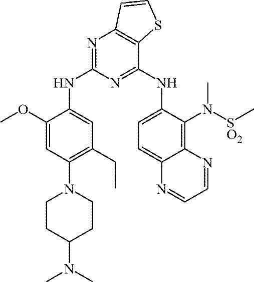 | ¹H NMR (600 MHz, MeOD) δ 9.01 (d, J = 1.8 Hz, 1H), 8.98 (d, J = 1.9 Hz, 1H), 8.28 (d, J = 5.5 Hz, 1H), 8.26 - 8.15 (m, 1H), 8.15 - 8.03 (m, 1H), 7.33 (d, J = 5.4 Hz, 1H), 7.25 (s, 1H), 6.78 (s, 1H), 3.82 (s, 3H), 3.46 (s, 3H), 3.36 - 3.32 (m, 2H), 3.17 - 3.07 (m, 5H), 2.93 (s, 6H), 2.85 -2.72 (m, 2H), 2.45 - 2.28 (m, 1H), 2.23 -2.15 (m, 2H), 1.94 - 1.83 (m, 2H), 0.76 (br s, 3H). MS (ESI) m/z: 662 [M+H]⁺. |
| II-1-17 | 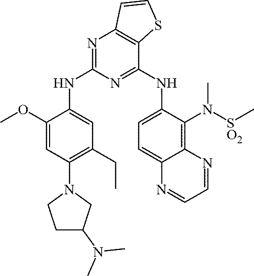 | ¹H NMR (600 MHz, MeOD) δ 9.06 - 9.02 (m, 1H), 9.01 - 8.98 (m, 1H), 8.30 (d, J = 5.5 Hz, 1H), 8.26 - 8.18 (m, 1H), 8.13 -8.06 (m, 1H), 7.35 (d, J = 5.5 Hz, 1H), 7.25 (d, J = 8.7 Hz, 1H), 6.78 (s, 1H), 4.09 -3.98 (m, 1H), 3.85 (s, 3H), 3.50 (s, 3H), 3.47 - 3.39 (m, 2H), 3.32 - 3.23 (m, 1H), 3.13 (s, 3H), 3.10 - 3.06 (m, 1H), 3.00 (s, 3H), 2.99 (s, 3H), 2.58 - 2.47 (m, 1H), 2.27 - 2.19 (m, 1H), 1.12 - 0.50 (m, 3H). MS (ESI) m/z: 648 [M+H]⁺. |
| II-1-18 | 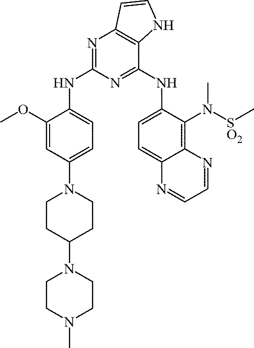 | ¹H NMR (600 MHz, MeOD) δ 8.99 (d, J = 1.8 Hz, 1H), 8.94 (d, J = 1.6 Hz, 1H), 8.42 (d, J = 9.2 Hz, 1H), 8.08 (d, J = 9.2 Hz, 1H), 7.64 (d, J = 3.0 Hz, 1H), 7.50 - 7.40 (m, 1H), 6.93 (s, 1H), 6.67 - 6.59 (m, 1H), 6.41 (d, J = 3.1 Hz, 1H), 3.84 (s, 3H), 3.83 - 3.78 (m, 2H), 3.63 - 3.47 (m, 7H), 3.46 -3.33 (m, 4H), 3.24 - 3.09 (m, 6H), 2.95 (s, 3H), 2.28 - 2.17 (m, 2H), 2.04 - 1.93 (m, 2H). MS (ESI) m/z: 672 [M+H]⁺. |
| II-1-19 | 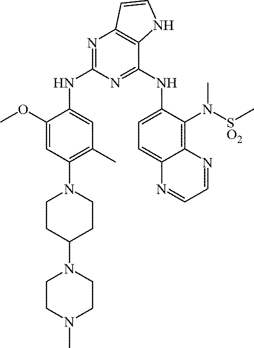 | ¹H NMR (600 MHz, MeOD) δ 8.99 (d, J = 1.8 Hz, 1H), 8.94 (d, J = 1.8 Hz, 1H), 8.37 (d, J = 9.2 Hz, 1H), 8.05 (d, J = 9.2 Hz, 1H), 7.64 (d, J = 3.1 Hz, 1H), 7.27 (s, 1H), 6.80 (s, 1H), 6.40 (d, J = 3.1 Hz, 1H), 3.82 (s, 3H), 3.68 - 3.54 (m, 8H), 3.52 (s, 3H), 3.50 - 3.45 (m, 1H), 3.29 - 3.25 (m, 2H), 3.15 (s, 3H), 2.98 (s, 3H), 2.88 (t, J = 11.9 Hz, 2H), 2.26 - 2.19 (m, 2H), 2.00 - 1.93 (m, 2H), 1.87 (br s, 3H). MS (ESI) m/z: 686 [M+H]⁺. |
| II-1-20 | 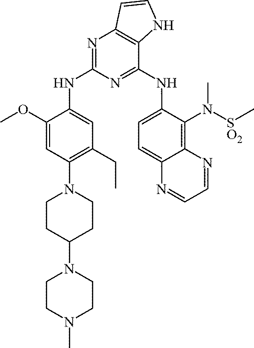 | ¹H NMR (600 MHz, MeOD) δ 9.01 - 8.97 (m, 1H), 8.96 - 8.91 (m, 1H), 8.31 (d, J = 9.0 Hz, 1H), 8.01 (d, J = 9.3 Hz, 1H), 7.63 (d, J = 3.2 Hz, 1H), 7.36 (s, 1H), 6.81 (s, 1H), 6.39 (d, J = 3.0 Hz, 1H), 3.82 (s, 3H), 3.76 - 3.59 (m, 8H), 3.52 (s, 3H), 3.41 -3.35 (m, 1H), 3.23 - 3.17 (m, 2H), 3.13 (s, 3H), 2.99 (s, 3H), 2.94 - 2.79 (m, 2H), 2.41 - 2.31 (m, 1H), 2.28 - 2.18 (m, 2H), 2.16 - 2.10 (m, 1H), 2.05 - 1.88 (m, 2H), 0.85 - 0.56 (m, 3H). MS (ESI) m/z: 670 [M+H]⁺. |
| II-1-21 | 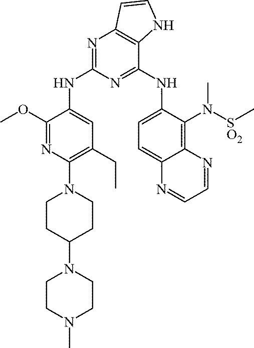 | ¹H NMR (600 MHz, Methanol-d₄) δ 9.00 (d, J = 1.8 Hz, 1H), 8.95 (d, J = 1.8 Hz, 1H), 8.27 (d, J = 9.0 Hz, 1H), 8.01 (d, J = 9.2 Hz, 1H), 7.66 (d, J = 3.1 Hz, 1H), 7.61 (s, 1H), 6.42 (d, J = 3.0 Hz, 1H), 3.91 (s, 3H), 3.72 - 3.53 (m, 11H), 3.50 - 3.42 (m, 2H), 3.37 - 3.33 (m, 1H), 3.14 (s, 3H), 3.01 - 2.88 (m, 5H), 2.40 - 2.24 (m, 1H), 2.23 - 2.15 (m, 2H), 2.13 - 2.03 (m, 1H), 2.01 - 1.86 (m, 2H), 0.84 - 0.45 (m, 3H). MS (ESI) m/z: 701 [M+H]⁺. |
| II-1-22 | 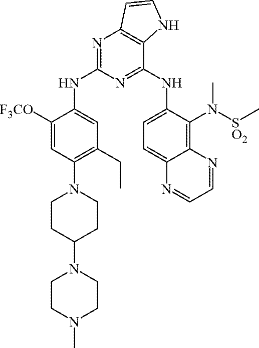 [SMILES: CCc1cc(Nc2nc(Nc3ccc4nccnc4c3N(C)S(C)(=O)=O)c3[nH]ccc3n2)c(OC(F)(F)F)cc1N1CCC(N2CCN(C)CC2)CC1] | ¹H NMR (600 MHz, Methanol-d₄) δ 9.00 (d, J = 1.9 Hz, 1H), 8.96 (d, J = 1.9 Hz, 1H), 8.18 (d, J = 9.2 Hz, 1H), 7.91 (d, J = 9.2 Hz, 1H), 7.69 (d, J = 3.0 Hz, 1H), 7.37 (s, 1H), 7.06 (s, 1H), 6.47 (d, J = 3.0 Hz, 1H), 3.70 - 3.56 (m, 8H), 3.52 (s, 3H), 3.32 - 3.27 (m, 1H), 3.19 - 3.12 (m, 5H), 2.98 (s, 3H), 2.84 - 2.72 (m, 2H), 2.50 -2.38 (m, 1H), 2.29 - 2.19 (m, 3H), 2.01 -1.86 (m, 2H), 0.76 (t, J = 7.5 Hz, 3H). MS (ESI) m/z: 754 [M+H]⁺. |
| II-1-23 | 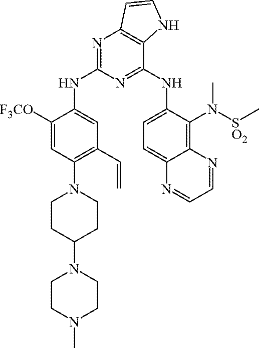 [SMILES: C=Cc1cc(Nc2nc(Nc3ccc4nccnc4c3N(C)S(C)(=O)=O)c3[nH]ccc3n2)c(OC(F)(F)F)cc1N1CCC(N2CCN(C)CC2)CC1] | ¹H NMR (600 MHz, Methanol-d₄) δ 8.96 (d, J = 1.8 Hz, 1H), 8.91 (d, J = 2.0 Hz, 1H), 8.08 (d, J = 9.2 Hz, 1H), 7.79 (d, J = 9.2 Hz, 1H), 7.67 (d, J = 3.0 Hz, 1H), 7.58 (s, 1H), 7.03 - 6.88 (m, 1H), 6.61 (dd, J = 17.7, 11.1 Hz, 1H), 6.45 (d, J = 3.1 Hz, 1H), 5.10 (d, J = 17.8 Hz, 1H), 4.72 (d, J = 11.2 Hz, 1H), 3.56 - 3.39 (m, 11H), 3.26 -3.20 (m, 2H), 3.18 - 3.10 (m, 4H), 2.91 (s, 3H), 2.81 - 2.66 (m, 2H), 2.27 - 2.10 (m, 2H), 1.95 - 1.82 (m, 2H). MS (ESI) m/z: 752 [M+H]⁺. |
| II-1-24 | 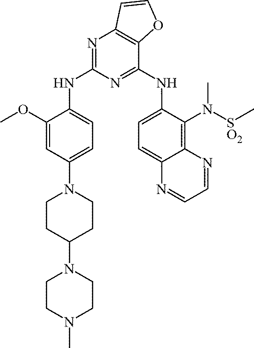 | ¹H NMR (600 MHz, MeOD) δ 9.00 (d, J = 1.8 Hz, 1H), 8.95 (d, J = 1.9 Hz, 1H), 8.43 (d, J = 9.3 Hz, 1H), 8.19 (d, J = 2.2 Hz, 1H), 8.12 (d, J = 9.2 Hz, 1H), 7.41 (d, J = 8.7 Hz, 1H), 6.95 - 6.87 (m, 2H), 6.68 -6.61 (m, 1H), 3.92 - 3.86 (m, 2H), 3.85 (s, 3H), 3.68 - 3.45 (m, 11H), 3.35 - 3.33 (m, 1H), 3.14 (s, 3H), 3.13 - 3.05 (m, 2H), 2.97 (s, 3H), 2.31 - 2.21 (m, 2H), 2.04 -1.87 (m, 2H). MS (ESI) m/z: 673 [M+H]⁺. |
| II-1-25 | 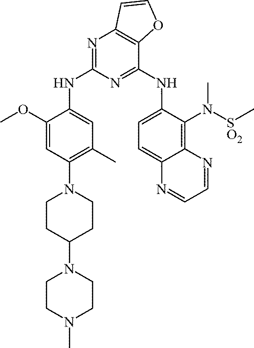 | ¹H NMR (600 MHz, MeOD) δ 9.00 (d, J = 1.7 Hz, 1H), 8.98 - 8.90 (m, 1H), 8.43 -8.29 (m, 1H), 8.19 (d, J = 2.2 Hz, 1H), 8.10 (d, J = 9.3 Hz, 1H), 7.23 (s, 1H), 6.99 -6.89 (m, 1H), 6.79 (s, 1H), 3.82 (s, 3H), 3.79 - 3.60 (m, 8H), 3.48 (s, 3H), 3.45 -3.38 (m, 1H), 3.29 - 3.23 (m, 2H), 3.13 (s, 3H), 3.01 (s, 3H), 2.90 - 2.79 (m, 2H), 2.31 - 2.20 (m, 2H), 2.07 - 1.85 (m, 5H). MS (ESI) m/z: 687 [M+H]⁺. |
| II-1-26 | 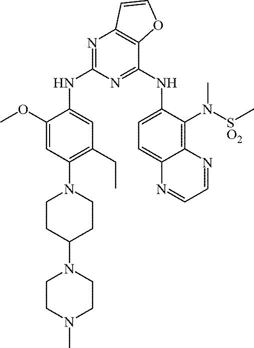 | ¹H NMR (600 MHz, MeOD) δ 9.01 (d, J = 1.8 Hz, 1H), 8.96 (d, J = 1.8 Hz, 1H), 8.37 - 8.27 (m, 1H), 8.20 (d, J = 2.2 Hz, 1H), 8.08 (d, J = 9.3 Hz, 1H), 7.29 (s, 1H), 7.01 - 6.90 (m, 1H), 6.82 (s, 1H), 3.83 (s, 3H), 3.79 - 3.62 (m, 8H), 3.48 (s, 3H), 3.45 -3.38 (m, 1H), 3.23 - 3.16 (m, 2H), 3.12 (s, 3H), 3.01 (s, 3H), 2.94 - 2.79 (m, 2H), 2.51 - 2.35 (m, 1H), 2.31 - 2.23 (m, 2H), 2.23 - 2.13 (m, 1H), 2.03 - 1.91 (m, 2H), 0.98 - 0.54 (m, 3H). MS (ESI) m/z: 701 [M+H]⁺. |
| II-1-27 | 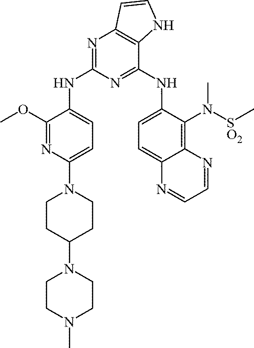 | ¹H NMR (600 MHz, Methanol-d₄) δ 8.98 (d, J = 1.8 Hz, 1H), 8.93 (d, J = 1.8 Hz, 1H), 8.64 - 8.29 (m, 1H), 8.17 - 7.95 (m, 1H), 7.60 (d, J = 3.0 Hz, 1H), 7.50 - 7.41 (m, 1H), 6.37 (d, J = 3.0 Hz, 1H), 6.30 -6.16 (m, 1H), 4.56 - 4.43 (m, 2H), 3.85 (s, 3H), 3.64 - 3.38 (m, 12H), 3.15 (s, 3H), 2.98 - 2.88 (m, 5H), 2.24 - 2.06 (m, 2H), 1.79 - 1.61 (m, 2H). MS (ESI) m/z: 673 [M+H]⁺. |
| II-1-28 | 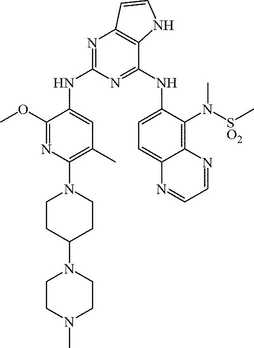 | ¹H NMR (600 MHz, Methanol-d₄) δ 8.99 (d, J = 1.8 Hz, 1H), 8.94 (d, J = 1.9 Hz, 1H), 8.64 - 8.29 (m, 1H), 8.21 - 7.94 (m, 1H), 7.62 (d, J = 3.0 Hz, 1H), 7.38 (s, 1H), 6.38 (d, J = 3.0 Hz, 1H), 3.93 (s, 3H), 3.86 - 3.77 (m, 1H), 3.74 - 3.65 (m, 1H), 3.59 (s, 3H), 3.53 - 3.32 (m, 6H), 3.27 - 3.11 (m, 6H), 2.96 - 2.83 (m, 4H), 2.77 - 2.65 (m, 1H), 2.16 - 1.95 (m, 4H), 1.91 - 1.83 (m, 1H), 1.56 - 1.22 (m, 2H). MS (ESI) m/z: 687 [M+H]⁺. |
| II-1-29 | 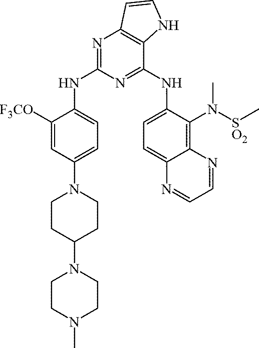 [SMILES: CN1CCN(C2CCN(c3ccc(Nc4nc(Nc5ccc6nccnc6c5N(C)S(C)(=O)=O)c5[nH]ccc5n4)c(OC(F)(F)F)c3)CC2)CC1] | ¹H NMR (600 MHz, Methanol-d₄) δ 8.97 (d, J = 2.3 Hz, 1H), 8.93 (d, J = 2.3 Hz, 1H), 8.31 (d, J = 9.5 Hz, 1H), 7.98 (d, J = 9.1 Hz, 1H), 7.65 (t, J = 2.7 Hz, 1H), 7.32 (dd, J = 9.0, 2.3 Hz, 1H), 6.93 (s, 1H), 6.84 (d, J = 8.9 Hz, 1H), 6.43 (t, J = 2.7 Hz, 1H), 3.92 - 3.81 (m, 2H), 3.64 - 3.46 (m, 11H), 3.29 - 3.25 (m, 1H), 3.14 (s, 3H), 2.95 (d, J = 2.3 Hz, 3H), 2.91 - 2.81 (m, 2H), 2.26 - 2.17 (m, 2H), 1.92 - 1.78 (m, 2H). MS (ESI) m/z: 726 [M+H]⁺. |
| II-1-30 | 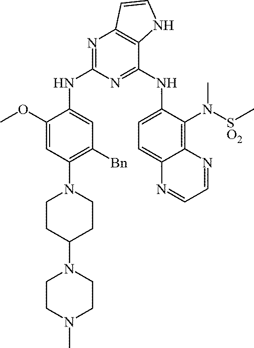 | ¹H NMR (600 MHz, Methanol-d₄) δ 8.90 (d, J = 2.0 Hz, 1H), 8.84 (d, J = 1.9 Hz, 1H), 8.32 (d, J = 9.2 Hz, 1H), 8.01 (d, J = 9.3 Hz, 1H), 7.65 (t, J = 2.2 Hz, 1H), 7.37 (s, 1H), 7.08 - 6.99 (m, 3H), 6.91 - 6.80 (m, 3H), 6.41 (d, J = 2.7 Hz, 1H), 3.83 (s, 3H), 3.73 - 3.61 (m, 1H), 3.54 - 3.35 (m, 12H), 3.12 - 3.06 (m, 4H), 3.01 - 2.95 (m, 1H), 2.93 - 2.88 (m, 4H), 2.79 - 2.64 (m, 2H), 2.10 - 1.94 (m, 2H), 1.78 - 1.68 (m, 2H). MS (ESI) m/z: 762 [M+H]⁺. |
| II-1-31 | 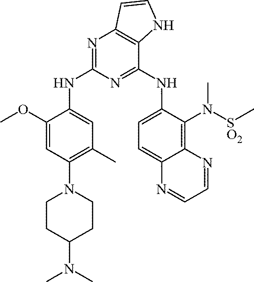 | ¹H NMR (600 MHz, Methanol-d₄) δ 8.99 (d, J = 1.8 Hz, 1H), 8.93 (d, J = 1.9 Hz, 1H), 8.38 (d, J = 9.1 Hz, 1H), 8.03 (d, J = 9.2 Hz, 1H), 7.62 (d, J = 2.9 Hz, 1H), 7.23 (s, 1H), 6.74 (s, 1H), 6.39 (d, J = 3.0 Hz, 1H), 3.80 (s, 3H), 3.52 (s, 3H), 3.38 - 3.33 (m, 1H), 3.25 - 3.20 (m, 2H), 3.15 (s, 3H), 2.94 (s, 6H), 2.84 - 2.75 (m, 2H), 2.26 -2.15 (m, 2H), 2.02 - 1.82 (m, 5H). MS (ESI) m/z: 631 [M+H]⁺. |
| II-1-32 | 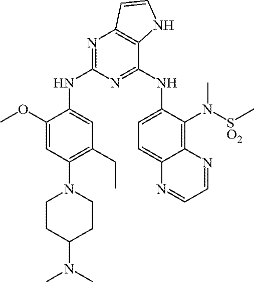 | ¹H NMR (600 MHz, Methanol-d₄) δ 8.99 (d, J = 1.9 Hz, 1H), 8.94 (d, J = 1.8 Hz, 1H), 8.34 (d, J = 9.2 Hz, 1H), 8.01 (d, J = 9.2 Hz, 1H), 7.63 (d, J = 3.0 Hz, 1H), 7.32 (s, 1H), 6.79 (s, 1H), 6.40 (d, J = 3.0 Hz, 1H), 3.82 (s, 3H), 3.53 (s, 3H), 3.36 - 3.32 (m, 1H), 3.17 - 3.11 (m, 5H), 2.93 (s, 6H), 2.87 - 2.76 (m, 2H), 2.44 - 2.33 (m, 1H), 2.25 - 2.13 (m, 3H), 1.93 - 1.84 (m, 2H), 0.85 - 0.61 (m, 3H). MS (ESI) m/z: 645 [M+H]⁺. |
| II-1-33 | 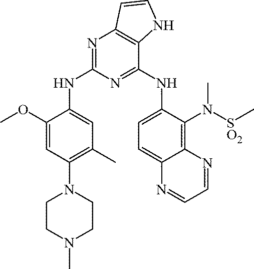 | ¹H NMR (600 MHz, Methanol-d₄) δ 8.99 (d, J = 1.9 Hz, 1H), 8.93 (d, J = 2.0 Hz, 1H), 8.36 (d, J = 9.2 Hz, 1H), 8.02 (dd, J = 9.2, 2.5 Hz, 1H), 7.63 (d, J = 2.7 Hz, 1H), 7.29 (s, 1H), 6.76 (s, 1H), 6.40 (d, J = 2.8 Hz, 1H), 3.82 (d, J = 2.1 Hz, 3H), 3.61 (t, J = 10.3 Hz, 2H), 3.52 (d, J = 2.1 Hz, 3H), 3.36 - 3.32 (m, 2H), 3.26 - 3.20 (m, 2H), 3.15 (d, J = 2.0 Hz, 3H), 3.10 (t, J = 12.6 Hz, 2H), 3.00 (d, J = 2.1 Hz, 3H), 1.87 (s, 3H). MS (ESI) m/z: 603 [M+H]⁺. |
| II-1-34 | 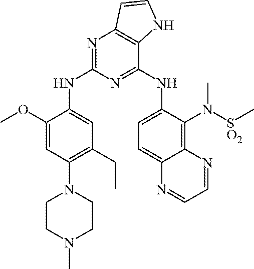 | ¹H NMR (600 MHz, Methanol-d₄) δ 9.00 (d, J = 1.9 Hz, 1H), 8.94 (d, J = 1.9 Hz, 1H), 8.31 (d, J = 9.2 Hz, 1H), 8.00 (d, J = 9.1 Hz, 1H), 7.65 (d, J = 3.1 Hz, 1H), 7.38 (s, 1H), 6.81 (s, 1H), 6.41 (d, J = 3.1 Hz, 1H), 3.84 (s, 3H), 3.66 - 3.58 (m, 2H), 3.53 (s, 3H), 3.31 - 3.26 (m, 2H), 3.22 -3.13 (m, 7H), 2.99 (s, 3H), 2.47 - 2.31 (m, 1H), 2.24 - 2.09 (m, 1H), 0.81 - 0.61 (m, 3H). MS (ESI) m/z: 617 [M+H]⁺. |
| II-1-35 | 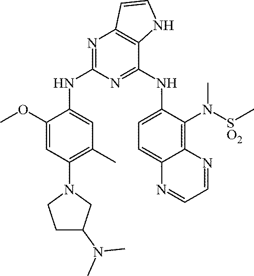 | ¹H NMR (600 MHz, Methanol-d₄) δ 8.99 (d, J = 1.9 Hz, 1H), 8.93 (d, J = 1.9 Hz, 1H), 8.39 (d, J = 9.1 Hz, 1H), 8.01 (d, J = 9.4 Hz, 1H), 7.63 (d, J = 3.1 Hz, 1H), 7.20 (d, J = 4.1 Hz, 1H), 6.71 (s, 1H), 6.40 (d, J = 3.0 Hz, 1H), 4.05 - 3.97 (m, 1H), 3.81 (s, 3H), 3.53 (d, J = 2.4 Hz, 3H), 3.48 - 3.43 (m, 1H), 3.43 - 3.36 (m, 2H), 3.15 (s, 3H), 3.14 - 3.09 (m, 1H), 2.99 (d, J = 3.4 Hz, 6H), 2.58 - 2.46 (m, 1H), 2.29 - 2.15 (m, 1H), 1.94 (s, 3H). MS (ESI) m/z: 617 [M+H]⁺. |
| II-1-36 | 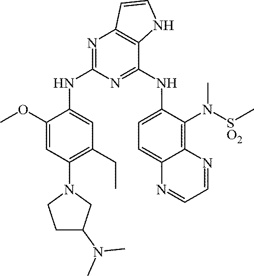 | ¹H NMR (600 MHz, Methanol-d₄) δ 8.99 (d, J = 1.9 Hz, 1H), 8.93 (d, J = 1.9 Hz, 1H), 8.41 - 8.28 (m, 1H), 7.99 (d, J = 9.3 Hz, 1H), 7.65 (d, J = 3.0 Hz, 1H), 7.29 (d, J = 8.0 Hz, 1H), 6.79 (s, 1H), 6.41 (d, J = 3.0 Hz, 1H), 4.08 - 3.98 (m, 1H), 3.82 (s, 3H), 3.54 (s, 3H), 3.45 - 3.38 (m, 2H), 3.37 - 3.32 (m, 1H), 3.14 (s, 3H), 3.12 -3.04 (m, 1H), 2.98 (d, J = 5.3 Hz, 6H), 2.53 - 2.46 (m, 1 H), 2.44 - 2.35 (m, 1 H), 2.30 -2.15 (m, 2H), 0.84 - 0.69 (m, 3H). MS (ESI) m/z: 631 [M+H]⁺. |
| II-1-37 | 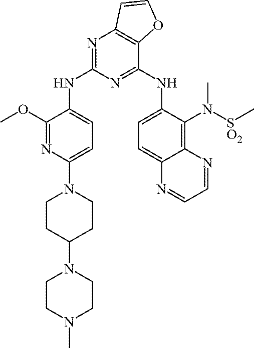 | ¹H NMR (600 MHz, Methanol-d₄) δ 8.99 (d, J = 1.8 Hz, 1H), 8.95 (d, J = 1.9 Hz, 1H), 8.64 - 8.31 (m, 1H), 8.17 (d, J = 2.3 Hz, 1H), 8.14 - 8.02 (m, 1H), 7.46 (dd, J = 9.2, 4.4 Hz, 1H), 7.03 - 6.76 (m, 1H), 6.46 - 6.15 (m, 1H), 4.60 - 4.44 (m, 2H), 3.87 (s, 3H), 3.54 (s, 11H), 3.43 - 3.36 (m, 1H), 3.14 (s, 3H), 3.00 - 2.86 (m, 5H), 2.27 -2.12 (m, 2H), 1.84 - 1.66 (m, 2H). MS (ESI) m/z: 674 [M+H]⁺. |
| II-1-38 | 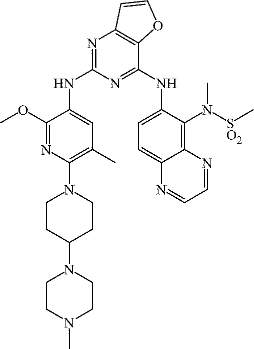 | ¹H NMR (600 MHz, Methanol-d₄) δ 9.00 (d, J = 1.8 Hz, 1H), 8.96 (d, J = 1.9 Hz, 1H), 8.45 - 8.31 (m, 1H), 8.19 (d, J = 2.2 Hz, 1H), 8.16 - 8.01 (m, 1H), 7.40 (s, 1H), 7.00 - 6.79 (m, 1H), 3.93 (s, 3H), 3.86 -3.70 (m, 2H), 3.63 - 3.42 (m, 11H), 3.30 -3.26 (m, 1H), 3.15 (s, 3H), 2.93 (s, 3H), 2.85 (dt, J = 26.2, 12.1 Hz, 2H), 2.21 -1.85 (m, 5H), 1.77 - 1.42 (m, 2H). MS (ESI) m/z: 688 [M+H]⁺. |
| II-1-39 | 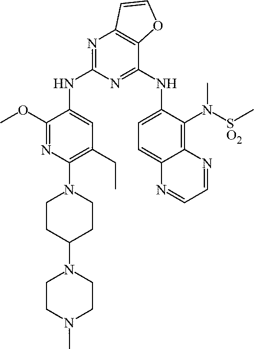 | ¹H NMR (600 MHz, Methanol-d₄) δ 9.09 -9.00 (m, 1H), 8.97 (d, J = 1.7 Hz, 1H), 8.36 - 8.19 (m, 2H), 8.07 (d, J = 9.3 Hz, 1H), 7.59 (s, 1H), 7.02 - 6.89 (m, 1H), 3.91 (s, 3H), 3.82 - 3.62 (m, 8H), 3.56 - 3.48 (m, 5H), 3.46 - 3.41 (m, 1H), 3.12 (s, 3H), 3.06 - 2.92 (m, 5H), 2.42 - 2.28 (m, 1H), 2.27 - 2.18 (m, 2H), 2.15 - 2.03 (m, 1H), 2.02 - 1.85 (m, 2H), 0.90 - 0.52 (m, 3H). MS (ESI) m/z: 702 [M+H]⁺. |
| II-1-40 | 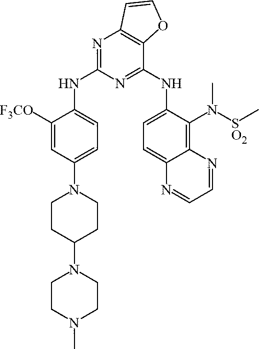 [SMILES: CN1CCN(C2CCN(c3ccc(Nc4nc(Nc5ccc6nccnc6c5N(C)S(C)(=O)=O)c5occc5n4)c(OC(F)(F)F)c3)CC2)CC1] | ¹H NMR (600 MHz, Methanol-d₄) δ 8.99 (d, J = 1.8 Hz, 1H), 8.95 (d, J = 1.7 Hz, 1H), 8.33 (d, J = 9.3 Hz, 1H), 8.20 (d, J = 2.1 Hz, 1H), 8.06 (d, J = 9.3 Hz, 1H), 7.36 (d, J = 8.9 Hz, 1H), 7.01 - 6.88 (m, 3H), 3.91 - 3.82 (m, 2H), 3.61 - 3.38 (m, 11H), 3.23 - 3.17 (m, 1H), 3.13 (s, 3H), 2.92 (s, 3H), 2.91 - 2.82 (m, 2H), 2.28 - 2.10 (m, 2H), 1.89 - 1.72 (m, 2H). MS (ESI) m/z: 727 [M+H]⁺. |
| II-1-41 | 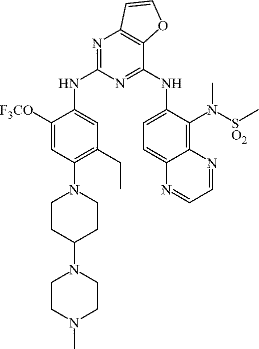 [SMILES: CCc1cc(Nc2nc(Nc3ccc4nccnc4c3N(C)S(C)(=O)=O)c3occc3n2)c(OC(F)(F)F)cc1N1CCC(N2CCN(C)CC2)CC1] | ¹H NMR (600 MHz, Methanol-d₄) δ 9.00 (d, J = 1.9 Hz, 1H), 8.96 (d, J = 1.9 Hz, 1H), 8.23 (d, J = 2.1 Hz, 1H), 8.16 (d, J = 9.2 Hz, 1H), 7.97 (d, J = 9.1 Hz, 1H), 7.37 (s, 1H), 7.05 (s, 1H), 6.97 (d, J = 2.2 Hz, 1H), 3.76 - 3.53 (m, 8H), 3.48 - 3.42 (m, 3H), 3.38 - 3.32 (m, 1H), 3.16 - 3.08 (m, 5H), 2.98 (s, 3H), 2.75 (dt, J = 17.6, 12.0 Hz, 2H), 2.51 - 2.40 (m, 1H), 2.28 - 2.16 (m, 3H), 1.98 - 1.84 (m, 2H), 0.83 (t, J = 7.4 Hz, 3H). MS (ESI) m/z: 755 [M+H]⁺. |
| II-1-42 | 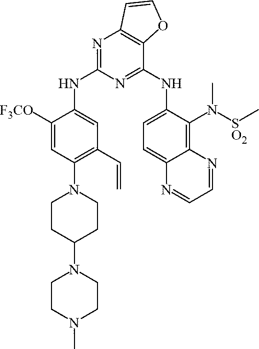 [SMILES: C=Cc1cc(Nc2nc(Nc3ccc4nccnc4c3N(C)S(C)(=O)=O)c3occc3n2)c(OC(F)(F)F)cc1N1CCC(N2CCN(C)CC2)CC1] | ¹H NMR (600 MHz, Methanol-d₄) δ 8.99 (d, J = 1.9 Hz, 1H), 8.96 (d, J = 1.9 Hz, 1H), 8.24 (d, J = 2.2 Hz, 1H), 8.09 (d, J = 10.0 Hz, 1H), 7.90 (d, J = 9.3 Hz, 1H), 7.60 (s, 1H), 7.05 - 6.93 (m, 2H), 6.63 (dd, J = 17.3, 11.4 Hz, 1H), 5.26 - 5.16 (m, 1H), 4.86 - 4.80 (m, 1H), 3.75 - 3.59 (m, 8H), 3.49 - 3.45 (m, 3H), 3.37 - 3.34 (m, 1H), 3.29 - 3.22 (m, 2H), 3.12 (s, 3H), 2.99 (s, 3H), 2.80 - 2.64 (m, 2H), 2.23 (t, J = 14.3 Hz, 2H), 2.01 - 1.87 (m, 2H). MS (ESI) m/z: 753 [M+H]⁺. |
| II-1-43 | 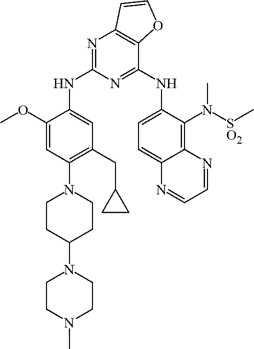 | ¹H NMR (600 MHz, Methanol-d₄) δ 9.01 (d, J = 1.9 Hz, 1H), 8.96 (d, J = 1.9 Hz, 1H), 8.46 - 8.25 (m, 1H), 8.20 (d, J = 2.2 Hz, 1H), 8.08 (d, J = 9.3 Hz, 1H), 7.47 (s, 1H), 6.92 (s, 1H), 6.83 (s, 1H), 3.84 (s, 3H), 3.50 (s, 3H), 3.48 - 3.34 (m, 8H), 3.20 - 3.11 (m, 6H), 2.89 (s, 3H), 2.86 -2.73 (m, 2H), 2.41 - 2.30 (m, 1H), 2.22 -2.00 (m, 3H), 1.95 - 1.71 (m, 2H), 0.74 -0.49 (m, 1H), 0.40 - 0.09 (m, 2H), -0.02 --0.25 (m, 2H). MS (ESI) m/z: 727 [M+H]⁺. |
| II-1-44 | 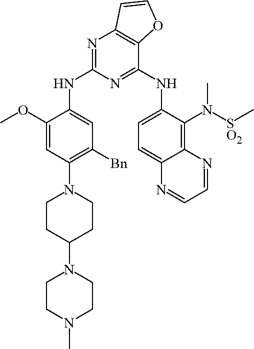 | ¹H NMR (600 MHz, Methanol-d₄) δ 8.93 (d, J = 1.7 Hz, 1H), 8.88 (d, J = 1.8 Hz, 1H), 8.39 - 8.25 (m, 1H), 8.20 (d, J = 2.2 Hz, 1H), 8.06 (d, J = 9.3 Hz, 1H), 7.40 -7.27 (m, 1H), 7.11 - 7.01 (m, 3H), 6.96 -6.79 (m, 4H), 3.83 (s, 3H), 3.75 - 3.54 (m, 10H), 3.45 (s, 3H), 3.31 - 3.25 (m, 1H), 3.08 (s, 3H), 3.02 - 2.90 (m, 5H), 2.84 -2.66 (m, 2H), 2.20 - 2.00 (m, 2H), 1.86 -1.73 (m, 2H). MS (ESI) m/z: 763 [M+H]⁺. |
| II-1-45 | 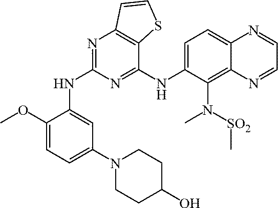 [SMILES: COc1ccc(N2CCC(O)CC2)cc1Nc1nc(Nc2ccc3nccnc3c2N(C)S(C)(=O)=O)c2sccc2n1] | ¹H NMR (600 MHz, Methanol-d₄) δ 9.02 (d, J = 1.7 Hz, 1H), 8.97 (d, J = 1.8 Hz, 1H), 8.27 (dd, J = 7.4, 4.2 Hz, 2H), 8.10 (d, J = 9.1 Hz, 1H), 8.02 (s, 1H), 7.38 (d, J = 5.5 Hz, 1H), 7.32 (dd, J = 9.1, 2.9 Hz, 1H), 7.20 (d, J = 8.9 Hz, 1H), 3.94 (s, 3H), 3.77 - 3.66 (m, 1H), 3.50 (s, 3H), 3.48 - 3.42 (m, 1H), 3.39 - 3.30 (m, 1H), 3.18 (s, 3H), 3.17 - 3.12 (m, 1H), 3.09 - 3.00 (m, 1H), 2.02 - 1.94 (m, 1H), 1.85 - 1.65 (m, 3H). MS (ESI) m/z: 607 [M+H]⁺. |
| II-1-46 | 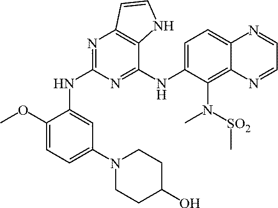 [SMILES: COc1ccc(N2CCC(O)CC2)cc1Nc1nc(Nc2ccc3nccnc3c2N(C)S(C)(=O)=O)c2[nH]ccc2n1] | ¹H NMR (600 MHz, Methanol-d₄) δ 9.02 (d, J = 1.9 Hz, 1H), 8.96 (d, J = 1.9 Hz, 1H), 8.26 (d, J = 9.2 Hz, 1H), 7.99 (d, J = 9.2 Hz, 1H), 7.84 (d, J = 2.9 Hz, 1H), 7.72 (d, J = 3.0 Hz, 1H), 7.30 (dd, J = 8.9, 2.9 Hz, 1H), 7.20 (d, J = 8.9 Hz, 1H), 6.49 (d, J = 3.0 Hz, 1H), 3.94 (s, 3H), 3.60 (s, 3H), 3.43 - 3.32 (m, 2H), 3.25 (s, 3H), 3.18 -3.07 (m, 1H), 3.03 - 2.94 (m, 1H), 2.82 -2.68 (m, 1H), 1.90 - 1.80 (m, 1H), 1.67 -1.43 (m, 3H). MS (ESI) m/z: 590 [M+H]⁺. |
| II-2-1 | 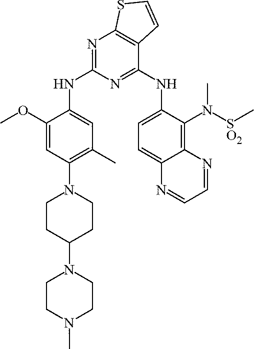 | ¹H NMR (600 MHz, MeOD) δ 8.87 (d, J = 1.9 Hz, 1H), 8.78 (d, J = 1.8 Hz, 1H), 8.56 (d, J = 9.3 Hz, 1H), 7.93 (d, J = 9.2 Hz, 1H), 7.54 (s, 1H), 7.34 (d, J = 5.8 Hz, 1H), 7.20 (d, J = 5.8 Hz, 1H), 6.72 (s, 1H), 3.75 (s, 3H), 3.59 - 3.34 (m, 11H), 3.31 - 3.27 (m, 1H), 3.21 - 3.13 (m, 2H), 3.12 (s, 3H), 2.98 - 2.92 (m, 2H), 2.91 (s, 3H), 2.20 -2.08 (m, 2H), 2.00 - 1.89 (m, 5H). MS (ESI) m/z: 703 [M+H]⁺. |
| II-2-2 | 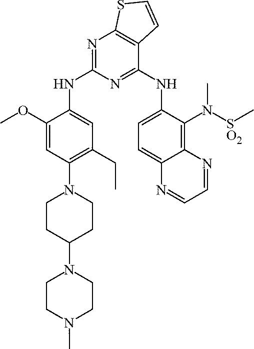 | ¹H NMR (600 MHz, MeOD) δ 8.99 (d, J = 1.8 Hz, 1H), 8.93 (d, J = 1.9 Hz, 1H), 8.47 (d, J = 9.2 Hz, 1H), 8.10 (d, J = 9.2 Hz, 1H), 7.58 (d, J = 5.8 Hz, 1H), 7.50 (s, 1H), 7.40 (d, J = 5.8 Hz, 1H), 6.86 (s, 1H), 3.84 (s, 3H), 3.68 - 3.52 (m, 8H), 3.47 (s, 3H), 3.37 - 3.33 (m, 1H), 3.28 - 3.19 (m, 2H), 3.15 (s, 3H), 3.02 - 2.93 (m, 5H), 2.52 -2.41 (m, 1H), 2.38 - 2.29 (m, 1H), 2.27 -2.15 (m, 2H), 2.09 - 1.93 (m, 2H), 1.03 -0.78 (m, 3H). MS (ESI) m/z: 717 [M+H]⁺. |
| II-2-3 | 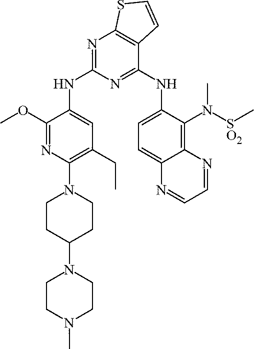 | ¹H NMR (600 MHz, DMSO-d₆) δ 9.31 (s, 1H), 9.02 (d, J = 1.9 Hz, 1H), 8.97 (d, J = 1.9 Hz, 1H), 8.57 (br s, 1H), 8.16 (s, 1H), 8.05 (d, J = 9.2 Hz, 1H), 7.86 (s, 1H), 7.57 (d, J = 5.9 Hz, 1H), 7.37 (d, J = 6.1 Hz, 1H), 3.85 (s, 3H), 3.78 - 3.18 (m, 14H), 3.17 (s, 3H), 2.88 (s, 3H), 2.85 - 2.74 (m, 2H), 2.41 - 2.25 (m, 1H), 2.23 - 2.14 (m, 1H), 2.16 - 2.03 (m, 2H), 1.86 - 1.65 (m, 2H), 0.94 - 0.63 (m, 3H). MS (ESI) m/z: 718 [M+H]⁺. |
| II-2-4 | 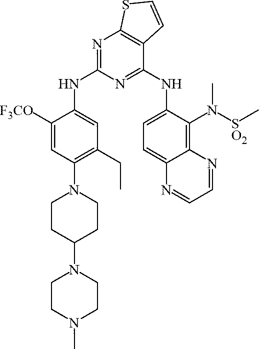 [SMILES: CCc1cc(Nc2nc(Nc3ccc4nccnc4c3N(C)S(C)(=O)=O)c3ccsc3n2)c(OC(F)(F)F)cc1N1CCC(N2CCN(C)CC2)CC1] | MS (ESI) m/z: 771 [M+H]⁺. |
| II-2-5 | 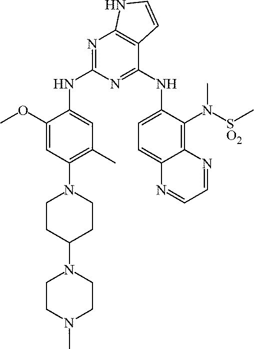 | ¹H NMR (600 MHz, MeOD) δ 8.98 (d, J = 1.9 Hz, 1H), 8.92 (d, J = 1.9 Hz, 1H), 8.40 (d, J = 9.3 Hz, 1H), 8.09 (d, J = 9.2 Hz, 1H), 7.57 (s, 1H), 7.05 (d, J = 3.6 Hz, 1H), 6.79 (s, 1H), 6.68 (d, J = 3.6 Hz, 1H), 3.83 (s, 3H), 3.53 - 3.39 (m, 11H), 3.27 - 3.22 (m, 2H), 3.18 - 3.12 (m, 4H), 2.95 - 2.88 (m, 5H), 2.18 - 2.14 (m, 2H), 1.97 - 1.86 (m, 5H). MS (ESI) m/z: 686 [M+H]⁺. |
| II-2-6 | 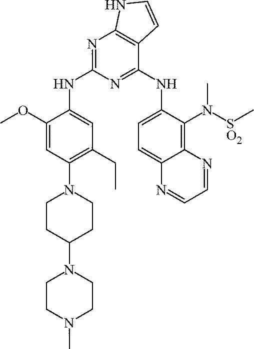 | MS (ESI) m/z: 700 [M+H]⁺. |
| II-2-7 | 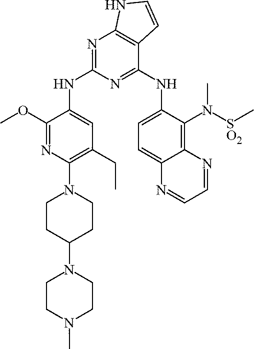 | ¹H NMR (600 MHz, MeOD) δ 8.96 (d, J =1.9 Hz, 1H), 8.90 (d, J = 1.8 Hz, 1H), 8.25 (d, J = 9.2 Hz, 1H), 8.03 (d, J = 9.1 Hz, 1H), 7.88 (s, 1H), 7.03 (d, J = 3.5 Hz, 1H), 6.69 (d, J = 3.6 Hz, 1H), 3.88 (s, 3H), 3.70 - 3.49 (m, 8H), 3.43 (s, 5H), 3.34 - 3.28 (m, 1H), 3.10 (s, 3H), 3.01 - 2.86 (m, 5H), 2.36 - 2.22 (m, 1H), 2.19 - 2.10 (m, 2H), 2.11 - 2.04 (m, 1H), 1.98 - 1.81 (m, 2H), 0.78 - 0.57 (m, 3H). MS (ESI) m/z: 701 [M+H]⁺. |
| II-2-8 | 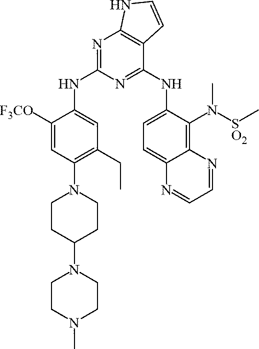 [SMILES: CCc1cc(Nc2nc(Nc3ccc4nccnc4c3N(C)S(C)(=O)=O)c3cc[nH]c3n2)c(OC(F)(F)F)cc1N1CCC(N2CCN(C)CC2)CC1] | ¹H NMR (600 MHz, MeOD) δ 8.97 (d, J = 1.9 Hz, 1H), 8.90 (d, J = 1.8 Hz, 1H), 8.35 (d, J = 9.2 Hz, 1H), 7.97 (d, J = 9.2 Hz, 1H), 7.66 (s, 1H), 7.08 (d, J = 3.6 Hz, 1H), 7.04 (s, 1H), 6.69 (d, J = 3.6 Hz, 1H), 3.44 (s, 3H), 3.43 - 3.31 (m, 8H), 3.15 (s, 3H), 3.13 - 3.02 (m, 3H), 2.87 (s, 3H), 2.79 -2.69 (m, 2H), 2.52 - 2.40 (m, 1H), 2.34 -2.24 (m, 1H), 2.17 - 2.09 (m, 2H), 1.90 -1.78 (m, 2H), 0.84 (t, J = 7.5 Hz, 3H). MS (ESI) m/z: 754 [M+H]⁺. |
| II-2-9 | 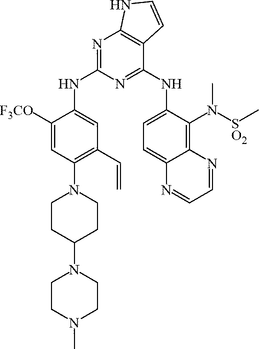 [SMILES: C=Cc1cc(Nc2nc(Nc3ccc4nccnc4c3N(C)S(C)(=O)=O)c3cc[nH]c3n2)c(OC(F)(F)F)cc1N1CCC(N2CCN(C)CC2)CC1] | ¹H NMR (600 MHz, MeOD) δ 8.95 - 8.91 (m, 1H), 8.87 (d, J = 1.7 Hz, 1H), 8.18 (d, J = 9.2 Hz, 1H), 7.85 (d, J = 9.1 Hz, 1H), 7.78 (s, 1H), 7.05 (d, J = 3.4 Hz, 1H), 6.95 (s, 1H), 6.70 (d, J = 3.5 Hz, 1H), 6.64 (dd, J = 17.7, 11.1 Hz, 1H), 5.17 (d, J = 17.7 Hz, 1H), 4.77 (d, J = 11.0 Hz, 1H), 3.63 -3.47 (m, 8H), 3.40 (s, 3H), 3.24 - 3.18 (m, 3H), 3.11 (s, 3H), 2.92 (s, 3H), 2.73 - 2.63 (m, 2H), 2.21 - 2.13 (m, 2H), 1.94 - 1.83 (m, 2H). MS (ESI) m/z: 752 [M+H]⁺. |
| II-2-10 | 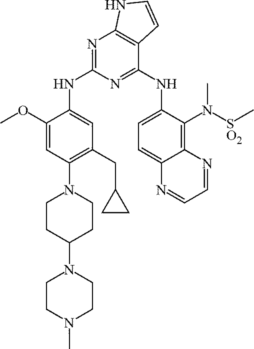 | MS (ESI) m/z: 726 [M+H]⁺. |
| II-2-11 | 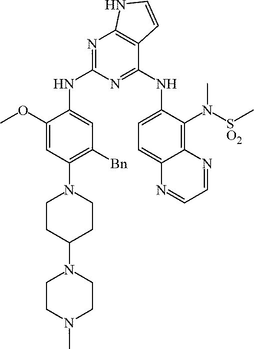 | MS (ESI) m/z: 762 [M+H]⁺. |
| II-2-12 | 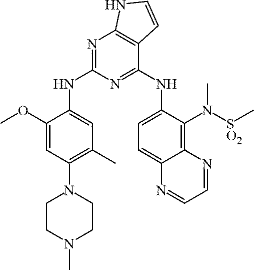 | ¹H NMR (600 MHz, MeOD) δ 8.98 (d, J = 1.9 Hz, 1H), 8.91 (d, J = 1.9 Hz, 1H), 8.48 (d, J = 9.2 Hz, 1H), 8.08 (d, J = 9.2 Hz, 1H), 7.66 (s, 1H), 7.05 (d, J = 3.5 Hz, 1H), 6.75 (s, 1H), 6.67 (d, J = 3.5 Hz, 1H), 3.85 (s, 3H), 3.64 - 3.56 (m, 2H), 3.46 (s, 3H), 3.38 - 3.31 (m, 2H), 3.25 - 3.19 (m, 2H), 3.17 (s, 3H), 3.12 - 3.05 (m, 2H), 2.98 (s, 3H), 1.92 (s, 3H). MS (ESI) m/z: 603 [M+H]⁺. |
| II-2-13 | 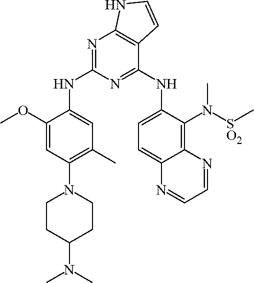 | MS (ESI) m/z: 631 [M+H]⁺. |
| II-2-14 | 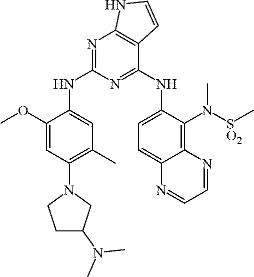 | MS (ESI) m/z: 617 [M+H]⁺. |
| II-2-15 | 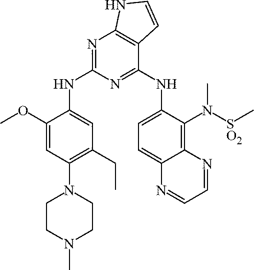 | MS (ESI) m/z: 617 [M+H]⁺. |
| II-2-16 | 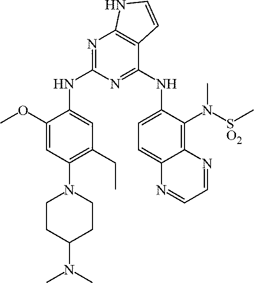 | ¹H NMR (600 MHz, Methanol-d₄) δ 8.98 (d, J = 1.9 Hz, 1H), 8.90 (d, J = 2.0 Hz, 1H), 8.63 - 8.50 (m, 1H), 8.07 (d, J = 9.4 Hz, 1H), 7.83 - 7.68 (m, 1H), 7.05 (d, J = 3.7 Hz, 1H), 6.79 (d, J = 6.1 Hz, 1H), 6.65 (d, J = 3.7 Hz, 1H), 3.86 (s, 3H), 3.52 -3.45 (m, 3H), 3.21 - 3.17 (m, 3H), 3.16 -3.08 (m, 3H), 2.94 (d, J = 3.3 Hz, 6H), 2.85 -2.81 (m, 2H), 2.20 - 2.17 (m, 2H), 1.92 -1.88 (m, 2H), 0.89 - 0.78 (m, 3H). MS (ESI) m/z: 645 [M+H]⁺. |
| II-2-17 | 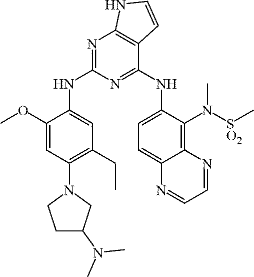 | ¹H NMR (600 MHz, Methanol-d₄) δ 8.96 (d, J = 1.9 Hz, 1H), 8.86 (d, J = 1.9 Hz, 1H), 8.78 - 8.73 (m, 1H), 8.05 (d, J = 9.3 Hz, 1H), 7.89 (s, 1H), 7.03 (d, J = 3.7 Hz, 1H), 6.81 (s, 1H), 6.58 (d, J = 3.6 Hz, 1H), 3.88 (s, 3H), 3.48 (s, 3H), 3.40 - 3.38 (m, 3H), 3.20 (s, 3H), 3.10 - 3.05 (m, 2H), 2.97 (s, 6H), 2.49 - 2.18 (m, 4H), 0.91 -0.88 (m, 3H). MS (ESI) m/z: 631 [M+H]⁺. |
| II-2-18 | 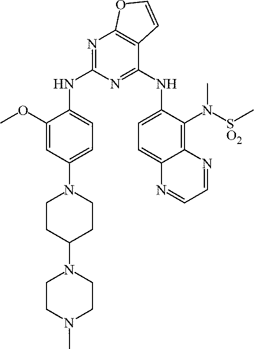 | MS (ESI) m/z: 673 [M+H]⁺. |
| II-2-19 | 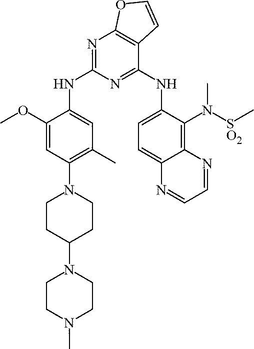 | MS (ESI) m/z: 687 [M+H]⁺. |
| II-2-20 | 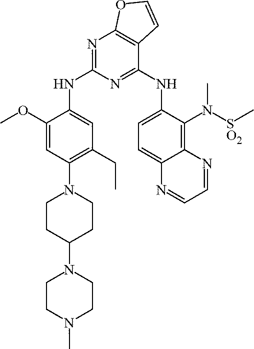 | MS (ESI) m/z: 701 [M+H]⁺. |
| II-2-21 | 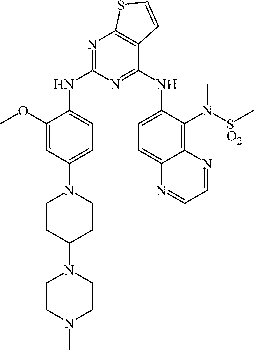 | ¹H NMR (600 MHz, Methanol-d₄) δ 8.90 -8.82 (m, 1H), 8.77 - 8.65 (m, 2H), 8.01 (d, J = 8.1 Hz, 1H), 7.84 (d, J = 9.2 Hz, 1H), 7.24 (d, J = 5.9 Hz, 1H), 7.16 (d, J = 5.9 Hz, 1H), 6.98 (s, 1H), 6.83 (d, J = 8.8 Hz, 1H), 3.84 (s, 3H), 3.75 - 3.68 (m, 2H), 3.52 - 3.34 (m, 9H), 3.27 - 3.12 (m, 6H), 3.10 - 3.04 (m, 1H), 2.95 (s, 3H), 2.22 (d, J = 13.1 Hz, 2H), 2.05 (q, J = 10.8 Hz, 2H). MS (ESI) m/z: 689 [M+H]⁺. |
| II-2-22 | 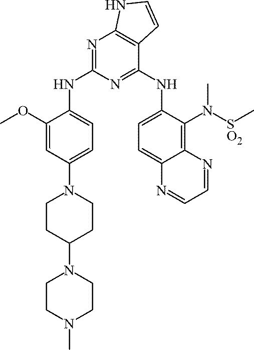 | ¹H NMR (600 MHz, Methanol-d₄) δ 8.99 (d, J = 1.9 Hz, 1H), 8.92 (d, J = 1.8 Hz, 1H), 8.52 (d, J = 9.3 Hz, 1H), 8.13 (d, J = 9.2 Hz, 1H), 7.98 (d, J = 8.6 Hz, 1H), 7.08 (d, J = 3.6 Hz, 1H), 7.03 (s, 1H), 6.81 -6.76 (m, 1H), 6.65 (d, J = 3.6 Hz, 1H), 3.90 (s, 3H), 3.83 - 3.73 (m, 2H), 3.61 - 3.34 (m, 9H), 3.30 - 3.27 (m, 1H), 3.26 - 3.13 (m, 6H), 3.11 - 3.04 (m, 1H), 2.93 (s, 3H), 2.26 - 2.18 (m, 2H), 2.06 - 1.95 (m, 2H). MS (ESI) m/z: 672 [M+H]⁺. |
| II-2-23 | 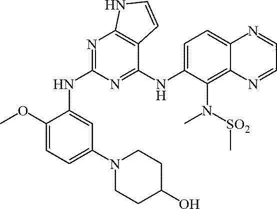 [SMILES: COc1ccc(N2CCC(O)CC2)cc1Nc1nc(Nc2ccc3nccnc3c2N(C)S(C)(=O)=O)c2cc[nH]c2n1] | MS (ESI) m/z: 590 [M+H]⁺. |

### Biological Activity Assay

### Growth Inhibitory Activity of Compounds on Cell Lines Stably Transfected With Kinase

The activity of compounds against various mutants of kinase EGFR was evaluated by their effect of inhibiting growth of BaF₃ cell lines stably transfected with kinase. The specific test method was given as follows:

1) Culture medium: DMEM (Dulbecco’s modified eagle medium) or RPMI 1640 (containing 10% fetal bovine serum, 100 µg/mL ampicillin, 100 µg/mL streptomycin).

2) Reagent: MTS reaction solution (containing 2 mg/mL of MTS [3-(4,5-dimethylthiazol-2-yl)-5-(3-carboxymethoxyphenyl)-2-(4-sulfophenyl)-2H-tetrazoliu m, inner Salt]; 100 µg/mL of PES (phenazine methosulfate)).

3) Compound test: cells were incubated into a 96-well culture plate, the volume of cytosol was 90 µL, and then 10 µL of the compound at each gradient concentration was added. That is, the highest concentration was 10 µM, which was diluted stepwise by ⅓, and 8 concentration points were set in total; 0.1% DMSO (dimethyl sulfoxide) was contained in the system. The cell plate with uniformly mixed compound was cultured in a cell culture incubator (37° C.; 5% CO₂) for 48 h, then 20 µL of MTS reaction solution was added, uniformly mixed and incubated in the cell culture incubator (37° C.; 5% CO₂) for 1-4 h. OD values at 490 nm were measured by a microplate reader (VARIOSKAN FLASH, Thermo). Two parallels were set in each group of experiments, with 0.1% (a final concentration) DMSO as a negative control, and a culture medium without cells and compounds as a blank control. The cell growth inhibition rate was calculated by the following formula:

$$\begin{matrix}\text{Cell growth inhibition rate\%=1} \text{-} \\ \left({\text{OD}}_{\text{experimental group}} {\text{-OD}}_{\text{blank group}}\right) / \left({\text{OD}}_{\text{negative group}} {\text{-OD}}_{\text{blank group}}\right) \\ \times 100 \% .\end{matrix}$$

4) IC₅₀ calculation: The semi-inhibitory concentration of the compounds acting on cells was calculated using GradPad Prism 5 software according to the measured cell growth inhibition rate.

The following table lists growth inhibitory activities of compounds on cell lines stably transfected with kinase:

| | | | | | | |
|---|---|---|---|---|---|---|
| No. | Δ19Del | L858R | Δ19Del/ T790M | L858R/ T790M | Δ19Del/T790M/ C797S | L858R/T790M/ C797S |
| I-1 | ++++ | ++++ | ++++ | ++++ | ++++ | ++++ |
| I-2 | ++++ | ++++ | ++++ | ++++ | ++++ | ++++ |
| I-3 | ++++ | ++++ | ++++ | ++++ | ++++ | ++++ |
| I-4 | +++ | +++ | +++ | +++ | ++++ | +++ |
| I-6 | +++ | ++++ | ++++ | ++++ | ++++ | ++++ |
| I-7 | +++ | +++ | +++ | +++ | ++++ | +++ |
| I-9 | ++++ | ++++ | ++++ | ++++ | ++++ | ++++ |
| I-10 | +++ | ++++ | ++++ | ++++ | ++++ | ++++ |
| I-11 | ++ | +++ | ++ | ++ | ++ | ++ |
| I-12 | +++ | ++++ | ++++ | ++++ | ++++ | ++++ |
| 1-13 | ++++ | ++++ | ++++ | ++++ | ++++ | ++++ |
| 1-14 | +++ | ++++ | ++++ | ++++ | ++++ | ++++ |
| I-15 | ++++ | ++++ | ++++ | ++++ | ++++ | ++++ |
| I-16 | ++++ | ++++ | ++++ | ++++ | ++++ | ++++ |
| I-17 | ++++ | ++++ | ++++ | ++++ | ++++ | ++++ |
| I-18 | +++ | +++ | +++ | ++++ | +++ | +++ |
| I-19 | ++++ | ++++ | ++++ | ++++ | ++++ | ++++ |
| I-20 | ++++ | ++++ | ++++ | ++++ | ++++ | ++++ |
| I-21 | +++ | ++++ | ++++ | ++++ | ++++ | ++++ |
| I-22 | +++ | +++ | ++++ | +++ | ++++ | ++++ |
| I-23 | ++++ | ++++ | ++++ | ++++ | ++++ | ++++ |
| I-24 | +++ | +++ | +++ | +++ | +++ | +++ |
| I-25 | ++++ | ++++ | +++ | ++++ | ++++ | ++++ |
| I-26 | ++++ | ++++ | ++++ | ++++ | ++++ | ++++ |
| I-27 | ++ | +++ | ++ | ++ | ++ | ++ |
| I-28 | +++ | +++ | +++ | +++ | +++ | +++ |
| I-29 | +++ | +++ | +++ | +++ | +++ | ++++ |
| I-30 | ++ | ++ | ++ | +++ | +++ | ++ |
| I-31 | +++ | ++++ | +++ | ++++ | ++++ | ++++ |
| I-32 | ++++ | ++++ | ++++ | ++++ | ++++ | ++++ |
| I-33 | +++ | ++++ | ++++ | ++++ | ++++ | +++ |
| I-34 | ++++ | ++++ | ++++ | ++++ | ++++ | ++++ |
| I-35 | ++++ | ++++ | ++++ | ++++ | ++++ | ++++ |
| I-37 | ++ | ++ | +++ | +++ | ++++ | +++ |
| I-38 | +++ | +++ | ++++ | ++++ | ++++ | ++++ |
| I-39 | ++ | ++ | +++ | +++ | ++++ | ++++ |
| I-40 | ++++ | ++++ | ++++ | ++++ | ++++ | ++++ |
| I-41 | ++++ | ++++ | ++++ | ++++ | ++++ | ++++ |
| I-43 | +++ | +++ | ++++ | ++++ | ++++ | ++++ |
| I-44 | +++ | +++ | ++++ | ++++ | ++++ | ++++ |
| I-45 | +++ | +++ | +++ | +++ | ++++ | ++++ |
| I-48 | +++ | +++ | +++ | +++ | ++++ | ++++ |
| I-49 | +++ | +++ | +++ | +++ | +++ | +++ |
| I-50 | +++ | +++ | +++ | ++++ | ++ | +++ |
| I-55 | +++ | +++ | +++ | +++ | +++ | +++ |
| I-56 | ++++ | ++++ | ++++ | ++++ | ++++ | ++++ |
| I-63 | ++++ | +++ | ++++ | ++++ | +++ | +++ |
| I-66 | +++ | +++ | +++ | ++++ | +++ | +++ |
| I-67 | ++++ | +++ | ++++ | ++++ | +++ | ++++ |
| I-73 | ++ | ++ | +++ | +++ | ++ | ++ |
| I-76 | +++ | ++ | +++ | ++++ | +++ | ++++ |
| I-77 | +++ | +++ | ++++ | ++++ | ++++ | ++++ |
| I-78 | +++ | +++ | +++ | ++++ | +++ | ++++ |
| I-80 | +++ | +++ | +++ | ++++ | ++ | +++ |
| I-81 | +++ | ++ | +++ | +++ | ++ | +++ |
| I-82 | +++ | +++ | +++ | ++++ | +++ | +++ |
| I-83 | +++ | +++ | ++ | +++ | ++ | ++ |
| I-84 | +++ | ++++ | +++ | ++++ | +++ | +++ |
| I-85 | +++ | +++ | +++ | +++ | +++ | +++ |
| I-87 | ++++ | ++++ | +++ | +++ | ++++ | ++++ |
| I-89 | ++ | +++ | +++ | +++ | ++ | +++ |
| I-90 | +++ | ++ | ++ | ++ | ++ | +++ |
| I-92 | +++ | +++ | +++ | +++ | +++ | ++++ |
| I-93 | ++++ | ++++ | ++++ | ++++ | ++++ | ++++ |
| I-94 | ++++ | ++++ | ++++ | ++++ | ++++ | ++++ |
| I-95 | ++++ | ++++ | ++++ | ++++ | ++++ | ++++ |
| I-96 | +++ | +++ | +++ | +++ | ++++ | ++++ |
| I-97 | ++++ | ++++ | ++++ | ++++ | ++++ | ++++ |
| I-98 | +++ | ++++ | ++++ | ++++ | ++++ | ++++ |
| I-99 | ++++ | ++++ | ++++ | ++++ | ++++ | ++++ |
| I-100 | ++++ | ++++ | ++++ | ++++ | ++++ | ++++ |
| I-101 | +++ | +++ | ++++ | +++ | ++++ | ++++ |
| I-102 | ++++ | ++++ | ++++ | ++++ | ++++ | ++++ |
| I-105 | ++++ | ++++ | ++++ | ++++ | ++++ | ++++ |
| I-106 | +++ | +++ | ++++ | +++ | ++++ | +++ |
| I-108 | +++ | +++ | ++++ | ++++ | ++++ | ++++ |
| I-110 | ++++ | ++++ | ++++ | ++++ | ++++ | ++++ |
| I-111 | +++ | +++ | +++ | ++++ | ++++ | +++ |
| I-112 | ++++ | ++++ | ++++ | ++++ | ++++ | ++++ |
| I-113 | +++ | ++++ | ++++ | ++++ | ++++ | ++++ |
| I-114 | ++++ | ++++ | ++++ | ++++ | ++++ | ++++ |
| I-115 | ++++ | ++++ | ++++ | ++++ | ++++ | ++++ |
| I-116 | +++ | +++ | +++ | +++ | ++++ | +++ |
| I-117 | ++++ | ++++ | ++++ | ++++ | ++++ | ++++ |
| I-118 | ++++ | ++++ | ++++ | ++++ | ++++ | ++++ |
| I-120 | ++++ | ++++ | ++++ | ++++ | ++++ | ++++ |
| I-121 | +++ | +++ | +++ | +++ | +++ | +++ |
| I-122 | ++++ | ++++ | ++++ | ++++ | ++++ | ++++ |
| I-123 | +++ | +++ | +++ | ++++ | ++++ | +++ |
| I-124 | ++ | ++ | +++ | +++ | ++++ | +++ |
| I-125 | ++ | +++ | +++ | +++ | ++++ | +++ |
| I-126 | ++ | ++ | +++ | ++ | +++ | +++ |
| I-127 | ++ | ++ | +++ | +++ | ++++ | +++ |
| I-128 | ++ | ++ | +++ | +++ | ++++ | +++ |
| I-129 | ++ | ++ | +++ | ++ | +++ | +++ |
| I-130 | +++ | +++ | ++++ | +++ | +++ | +++ |
| I-131 | ++ | ++ | +++ | ++ | +++ | +++ |
| I-132 | ++ | ++ | +++ | +++ | ++++ | +++ |
| I-133 | ++ | ++ | +++ | +++ | ++++ | +++ |
| I-134 | +++ | +++ | ++++ | ++++ | ++++ | ++++ |
| I-135 | +++ | +++ | ++++ | ++++ | ++++ | ++++ |
| I-137 | +++ | ++++ | ++++ | ++++ | ++++ | ++++ |
| I-138 | +++ | +++ | +++ | ++++ | ++++ | +++ |
| I-142 | ++++ | ++++ | ++++ | ++++ | ++++ | ++++ |
| I-143 | +++ | +++ | ++++ | +++ | ++++ | +++ |
| I-154 | ++++ | ++++ | ++++ | ++++ | ++++ | ++++ |
| I-155 | +++ | ++++ | ++++ | ++++ | ++++ | ++++ |
| I-156 | +++ | +++ | ++++ | +++ | ++++ | +++ |
| I-157 | ++++ | ++++ | ++++ | ++++ | ++++ | ++++ |
| I-158 | +++ | +++ | ++++ | ++++ | ++++ | ++++ |
| I-159 | ++++ | ++++ | ++++ | ++++ | ++++ | ++++ |
| I-160 | ++++ | +++ | ++++ | ++++ | ++++ | ++++ |
| I-161 | +++ | +++ | +++ | +++ | +++ | +++ |
| I-162 | ++++ | ++++ | ++++ | ++++ | ++++ | ++++ |
| I-163 | ++++ | ++++ | ++++ | ++++ | ++++ | ++++ |
| I-164 | ++ | +++ | ++ | +++ | +++ | +++ |
| I-166 | +++ | +++ | +++ | +++ | ++++ | +++ |
| I-167 | +++ | ++++ | ++++ | ++++ | ++++ | ++++ |
| I-168 | ++ | ++ | +++ | ++ | ++ | ++ |
| I-169 | ++++ | ++++ | ++++ | ++++ | ++++ | ++++ |
| I-170 | ++++ | ++++ | ++++ | ++++ | ++++ | ++++ |
| I-171 | +++ | ++++ | ++++ | ++++ | ++++ | ++++ |
| I-172 | ++++ | ++++ | ++++ | ++++ | ++++ | ++++ |
| I-173 | ++++ | ++++ | ++++ | ++++ | ++++ | ++++ |
| I-174 | ++++ | ++++ | ++++ | ++++ | ++++ | ++++ |
| I-175 | ++ | ++ | ++ | ++ | +++ | ++ |
| I-177 | ++++ | ++++ | ++++ | ++++ | ++++ | ++++ |
| I-178 | +++ | +++ | ++ | ++ | +++ | ++ |
| I-179 | +++ | +++ | +++ | +++ | ++++ | +++ |
| I-180 | ++++ | ++++ | +++ | ++++ | ++++ | +++ |
| I-181 | ++++ | ++++ | +++ | ++++ | ++++ | +++ |
| 1-182 | ++ | ++ | ++ | ++ | ++ | ++ |
| I-183 | ++++ | ++++ | +++ | ++++ | ++++ | ++++ |
| I-184 | ++++ | ++++ | +++ | ++++ | ++++ | ++++ |
| I-185 | +++ | ++++ | +++ | +++ | ++++ | +++ |
| I-186 | +++ | ++++ | +++ | +++ | ++++ | ++++ |
| I-187 | ++++ | +++ | +++ | +++ | +++ | +++ |
| I-188 | +++ | ++++ | ++++ | +++ | ++++ | +++ |
| I-190 | +++ | +++ | ++++ | ++++ | ++++ | +++ |
| I-191 | +++ | +++ | +++ | +++ | ++++ | +++ |
| I-192 | ++++ | +++ | ++++ | ++++ | ++++ | ++++ |
| I-193 | ++++ | ++++ | ++++ | ++++ | ++++ | ++++ |
| I-194 | +++ | +++ | +++ | +++ | ++++ | +++ |
| I-195 | +++ | +++ | +++ | +++ | ++++ | +++ |
| I-196 | +++ | +++ | +++ | +++ | ++++ | +++ |
| I-197 | +++ | +++ | +++ | +++ | ++++ | +++ |
| I-199 | +++ | ++++ | ++++ | ++++ | ++++ | ++++ |
| I-200 | +++ | +++ | +++ | +++ | +++ | +++ |
| I-201 | +++ | +++ | +++ | +++ | ++++ | +++ |
| I-202 | +++ | +++ | ++++ | ++++ | ++++ | +++ |
| I-203 | +++ | +++ | ++++ | ++++ | ++++ | +++ |
| I-204 | ++ | ++ | ++ | ++ | ++ | ++ |
| I-205 | ++++ | ++++ | ++++ | ++++ | ++++ | ++++ |
| I-206 | ++++ | ++++ | ++++ | ++++ | ++++ | ++++ |
| I-207 | +++ | +++ | ++++ | ++++ | ++++ | ++++ |
| I-208 | +++ | ++++ | ++++ | ++++ | ++++ | ++++ |
| I-209 | +++ | ++++ | ++++ | ++++ | ++++ | ++++ |
| I-210 | ++++ | ++++ | ++++ | ++++ | ++++ | ++++ |
| I-211 | +++ | ++++ | ++++ | ++++ | ++++ | ++++ |
| I-212 | +++ | +++ | ++++ | ++++ | ++++ | ++++ |
| I-213 | ++++ | ++++ | ++++ | ++++ | ++++ | ++++ |
| I-214 | +++ | +++ | ++++ | ++++ | ++++ | +++ |
| I-215 | +++ | +++ | ++++ | ++++ | ++++ | ++++ |
| I-216 | ++ | ++ | ++ | ++ | +++ | +++ |
| I-218 | +++ | +++ | ++++ | ++++ | ++++ | ++++ |
| I-219 | +++ | +++ | ++++ | +++ | ++++ | +++ |
| I-220 | +++ | +++ | +++ | +++ | ++++ | ++++ |
| I-221 | +++ | +++ | ++++ | ++++ | ++++ | ++++ |
| I-222 | +++ | +++ | ++++ | ++++ | ++++ | ++++ |
| I-223 | +++ | +++ | ++++ | ++++ | ++++ | ++++ |
| I-224 | +++ | +++ | +++ | ++++ | ++++ | ++++ |
| I-225 | +++ | +++ | +++ | +++ | ++++ | +++ |
| I-226 | ++ | ++ | +++ | +++ | ++ | ++ |
| I-227 | +++ | ++++ | ++++ | ++++ | ++++ | ++++ |
| I-228 | +++ | +++ | ++++ | ++++ | ++++ | ++++ |
| I-229 | +++ | +++ | +++ | ++++ | ++++ | ++++ |
| I-230 | ++++ | ++++ | ++++ | ++++ | ++++ | ++++ |
| I-231 | ++++ | ++++ | +++ | ++++ | ++++ | ++++ |
| I-232 | ++++ | ++++ | ++++ | ++++ | ++++ | ++++ |
| I-233 | ++++ | ++++ | ++++ | ++++ | ++++ | ++++ |
| I-234 | +++ | +++ | ++++ | ++++ | ++++ | ++++ |
| I-235 | +++ | +++ | ++++ | ++++ | ++++ | +++ |
| I-236 | ++++ | ++++ | ++++ | ++++ | ++++ | ++++ |
| I-237 | +++ | +++ | +++ | +++ | +++ | +++ |
| I-238 | +++ | +++ | +++ | +++ | ++++ | +++ |
| I-239 | +++ | +++ | +++ | +++ | ++++ | +++ |
| I-240 | ++ | ++ | ++ | ++ | ++ | ++ |
| I-241 | ++++ | ++++ | ++++ | ++++ | ++++ | +++ |
| I-242 | +++ | +++ | +++ | +++ | ++++ | ++++ |
| I-243 | ++++ | ++++ | ++++ | ++++ | +++ | +++ |
| I-244 | ++++ | ++++ | +++ | ++++ | ++++ | ++++ |
| I-245 | ++++ | ++++ | ++++ | ++++ | ++++ | ++++ |
| I-246 | ++++ | ++++ | ++++ | ++++ | ++++ | ++++ |
| I-247 | +++ | +++ | ++ | ++ | +++ | ++ |
| I-248 | ++++ | ++++ | +++ | ++++ | ++++ | +++ |
| I-249 | ++++ | ++++ | +++ | ++++ | ++++ | +++ |
| I-250 | +++ | +++ | ++ | +++ | +++ | ++ |
| I-251 | +++ | +++ | ++ | +++ | +++ | ++ |
| I-252 | ++++ | ++++ | +++ | ++++ | ++++ | +++ |
| I-253 | +++ | +++ | ++++ | +++ | ++++ | +++ |
| I-254 | +++ | +++ | ++++ | +++ | +++ | +++ |
| I-255 | +++ | +++ | ++++ | +++ | ++++ | +++ |
| I-256 | ++ | ++ | ++ | +++ | +++ | +++ |
| I-257 | ++ | ++ | +++ | +++ | ++++ | +++ |
| I-258 | ++ | ++ | +++ | +++ | ++++ | +++ |
| I-259 | ++ | ++ | ++ | ++ | +++ | +++ |
| I-260 | ++ | ++ | +++ | +++ | ++++ | +++ |
| I-261 | ++ | ++ | +++ | +++ | +++ | +++ |
| I-262 | ++ | ++ | +++ | ++ | +++ | +++ |
| I-263 | ++ | ++ | +++ | ++ | +++ | +++ |
| I-264 | ++ | ++ | ++ | ++ | +++ | +++ |
| I-265 | ++ | ++ | ++ | ++ | +++ | +++ |
| I-266 | ++ | ++ | ++ | ++ | ++ | +++ |
| I-267 | +++ | +++ | +++ | +++ | +++ | +++ |
| I-268 | ++ | ++ | ++ | ++ | +++ | +++ |
| I-269 | ++++ | ++++ | ++++ | ++++ | ++++ | ++++ |
| I-270 | +++ | ++++ | +++ | +++ | ++++ | ++++ |
| I-271 | +++ | +++ | +++ | +++ | ++++ | ++++ |
| I-272 | ++++ | ++++ | ++++ | ++++ | ++++ | ++++ |
| I-273 | ++++ | ++++ | ++++ | ++++ | ++++ | ++++ |
| I-274 | +++ | +++ | +++ | +++ | ++++ | +++ |
| I-275 | ++++ | ++++ | ++++ | +++ | ++++ | ++++ |
| I-276 | ++++ | ++++ | ++++ | ++++ | ++++ | ++++ |
| I-277 | ++++ | ++++ | ++++ | ++++ | ++++ | ++++ |
| I-278 | ++++ | ++++ | ++++ | ++++ | ++++ | ++++ |
| I-279 | ++++ | ++++ | ++++ | ++++ | ++++ | ++++ |
| I-280 | ++++ | ++++ | ++++ | ++++ | ++++ | ++++ |
| I-281 | ++++ | ++++ | ++++ | ++++ | ++++ | ++++ |
| I-282 | ++++ | ++++ | ++++ | ++++ | ++++ | ++++ |
| I-283 | ++++ | ++++ | ++++ | ++++ | ++++ | ++++ |
| I-284 | +++ | ++ | +++ | +++ | ++++ | ++++ |
| I-285 | +++ | +++ | ++++ | ++++ | ++++ | ++++ |
| I-286 | ++++ | ++++ | ++++ | ++++ | ++++ | ++++ |
| I-287 | +++ | +++ | ++++ | ++++ | ++++ | ++++ |
| I-288 | +++ | ++++ | +++ | +++ | +++ | +++ |
| I-289 | ++++ | ++++ | ++++ | ++++ | ++++ | ++++ |
| I-290 | ++ | ++ | +++ | +++ | +++ | +++ |
| I-291 | +++ | +++ | +++ | +++ | +++ | ++ |
| I-292 | +++ | +++ | ++++ | ++++ | +++ | +++ |
| I-293 | +++ | +++ | ++++ | ++++ | +++ | +++ |
| I-294 | +++ | +++ | +++ | +++ | ++++ | +++ |
| I-295 | +++ | +++ | +++ | +++ | ++++ | +++ |
| I-296 | +++ | +++ | +++ | +++ | ++ | +++ |
| I-297 | ++ | +++ | +++ | +++ | ++ | ++ |
| I-298 | +++ | +++ | ++++ | +++ | ++++ | ++++ |
| I-299 | ++++ | +++ | ++++ | ++++ | ++++ | ++++ |
| I-300 | +++ | +++ | +++ | ++++ | ++++ | ++++ |
| I-301 | +++ | ++++ | ++++ | ++++ | ++++ | ++++ |
| I-302 | +++ | +++ | ++++ | ++++ | +++ | ++++ |
| I-303 | +++ | +++ | +++ | +++ | ++++ | ++++ |
| I-304 | +++ | +++ | ++++ | ++++ | +++ | +++ |
| I-305 | +++ | +++ | +++ | ++++ | +++ | +++ |
| I-306 | ++ | +++ | +++ | +++ | ++ | ++ |
| I-307 | ++ | ++ | +++ | +++ | +++ | +++ |
| I-308 | +++ | +++ | ++++ | ++++ | ++++ | +++ |
| I-309 | +++ | ++++ | ++++ | ++++ | ++++ | ++++ |
| I-310 | +++ | ++++ | ++++ | ++++ | ++++ | ++++ |
| I-311 | +++ | +++ | ++++ | ++++ | ++++ | ++++ |
| I-312 | +++ | +++ | ++++ | ++++ | ++++ | ++++ |
| I-313 | +++ | +++ | +++ | +++ | ++++ | +++ |
| I-314 | +++ | +++ | ++++ | ++++ | ++++ | ++++ |
| I-315 | ++++ | ++++ | ++++ | ++++ | ++++ | ++++ |
| I-316 | +++ | +++ | +++ | +++ | ++++ | +++ |
| I-317 | ++++ | ++++ | ++++ | ++++ | ++++ | ++++ |
| I-318 | ++++ | ++++ | ++++ | ++++ | ++++ | ++++ |
| I-319 | ++++ | ++++ | ++++ | ++++ | ++++ | ++++ |
| I-320 | ++++ | ++++ | ++++ | ++++ | ++++ | ++++ |
| I-321 | +++ | +++ | ++++ | +++ | ++++ | +++ |
| I-322 | ++++ | ++++ | ++++ | ++++ | ++++ | ++++ |
| I-323 | ++++ | ++++ | ++++ | ++++ | ++++ | ++++ |
| I-324 | ++++ | ++++ | ++++ | ++++ | ++++ | ++++ |
| I-325 | +++ | +++ | +++ | +++ | ++++ | +++ |
| I-326 | +++ | +++ | +++ | +++ | +++ | +++ |
| I-327 | ++++ | ++++ | ++++ | ++++ | +++ | +++ |
| I-328 | +++ | +++ | ++++ | ++++ | +++ | +++ |
| I-329 | +++ | +++ | ++++ | ++++ | +++ | +++ |
| I-330 | ++++ | ++++ | ++++ | ++++ | ++++ | ++++ |
| I-331 | +++ | +++ | ++++ | +++ | ++++ | ++++ |
| I-332 | ++++ | ++++ | ++++ | ++++ | ++++ | ++++ |
| I-333 | ++++ | ++++ | ++++ | ++++ | ++++ | ++++ |
| I-334 | ++++ | ++++ | ++++ | ++++ | ++++ | ++++ |
| I-335 | ++++ | ++++ | ++++ | ++++ | ++++ | ++++ |
| I-336 | +++ | +++ | +++ | +++ | ++ | +++ |
| I-337 | ++++ | +++ | ++++ | +++ | +++ | +++ |
| I-338 | +++ | +++ | +++ | +++ | ++ | +++ |
| I-340 | ++++ | ++++ | ++++ | ++++ | +++ | +++ |
| I-341 | ++++ | ++++ | ++++ | ++++ | +++ | ++++ |
| I-342 | +++ | +++ | +++ | +++ | ++ | ++ |
| I-343 | +++ | +++ | +++ | +++ | +++ | +++ |
| I-344 | ++++ | ++++ | +++ | ++++ | +++ | ++++ |
| I-345 | +++ | +++ | +++ | +++ | +++ | +++ |
| I-346 | ++++ | ++++ | ++++ | ++++ | +++ | ++++ |
| I-347 | ++ | +++ | ++ | +++ | +++ | +++ |
| I-348 | +++ | +++ | ++++ | +++ | +++ | +++ |
| I-349 | +++ | ++++ | ++++ | ++++ | ++++ | ++++ |
| I-350 | +++ | +++ | +++ | +++ | +++ | +++ |
| I-351 | +++ | +++ | +++ | ++++ | +++ | +++ |
| I-352 | +++ | +++ | +++ | ++++ | +++ | +++ |
| I-353 | +++ | ++++ | ++++ | ++++ | ++++ | ++++ |
| I-354 | +++ | ++++ | ++++ | ++++ | ++++ | ++++ |
| I-355 | +++ | +++ | +++ | +++ | ++++ | +++ |
| I-356 | +++ | ++++ | +++ | ++++ | ++++ | +++ |
| I-357 | +++ | ++++ | ++++ | ++++ | ++++ | ++++ |
| I-358 | ++ | +++ | +++ | +++ | +++ | +++ |
| I-359 | ++++ | ++++ | ++++ | ++++ | ++++ | ++++ |
| I-360 | +++ | ++++ | +++ | ++++ | +++ | +++ |
| I-361 | ++ | ++ | ++ | +++ | ++ | ++ |
| I-362 | +++ | ++++ | +++ | +++ | ++++ | ++++ |
| I-363 | ++++ | ++++ | +++ | ++++ | ++++ | ++++ |
| I-364 | ++++ | +++ | ++++ | +++ | +++ | ++++ |
| I-365 | +++ | ++++ | +++ | ++++ | ++++ | ++++ |
| I-366 | +++ | +++ | +++ | ++++ | ++++ | ++++ |
| I-367 | ++ | ++ | ++ | ++ | +++ | +++ |
| I-368 | +++ | +++ | +++ | ++++ | ++++ | ++++ |
| I-369 | +++ | +++ | ++++ | ++++ | ++++ | ++++ |
| I-370 | ++ | +++ | ++ | +++ | ++++ | ++++ |
| I-371 | ++ | +++ | +++ | +++ | ++++ | ++++ |
| I-372 | +++ | ++++ | +++ | ++++ | ++++ | ++++ |
| I-373 | ++ | +++ | ++ | ++ | +++ | ++ |
| I-374 | +++ | +++ | +++ | +++ | ++++ | ++++ |
| I-375 | ++++ | ++++ | ++++ | ++++ | ++++ | ++++ |
| I-376 | +++ | +++ | +++ | ++++ | ++++ | ++++ |
| I-377 | ++++ | ++++ | ++++ | ++++ | ++++ | ++++ |
| I-378 | +++ | +++ | ++++ | ++++ | ++++ | ++++ |
| I-379 | ++++ | ++++ | ++++ | ++++ | ++++ | ++++ |
| I-380 | ++++ | ++++ | ++++ | ++++ | ++++ | ++++ |
| I-381 | +++ | +++ | ++++ | ++++ | +++ | ++++ |
| I-382 | +++ | +++ | +++ | ++++ | +++ | ++++ |
| I-383 | +++ | +++ | +++ | +++ | +++ | ++++ |
| I-384 | ++ | ++ | +++ | +++ | +++ | +++ |
| I-385 | ++ | ++ | ++ | ++ | +++ | ++++ |
| I-386 | +++ | +++ | +++ | +++ | ++++ | +++ |
| I-387 | ++ | ++ | ++ | +++ | +++ | +++ |
| I-388 | +++ | +++ | +++ | ++++ | ++++ | ++++ |
| I-389 | ++ | ++ | ++ | ++ | ++ | +++ |
| I-390 | ++ | +++ | +++ | +++ | +++ | ++++ |
| I-391 | +++ | +++ | +++ | +++ | ++++ | ++++ |
| I-392 | +++ | +++ | +++ | +++ | ++++ | ++++ |
| I-396 | +++ | +++ | +++ | +++ | ++++ | ++++ |
| I-398 | ++++ | ++++ | ++++ | ++++ | ++++ | ++++ |
| I-399 | +++ | +++ | +++ | +++ | ++++ | ++++ |
| I-401 | +++ | +++ | +++ | ++++ | +++ | +++ |
| I-402 | ++++ | +++ | ++++ | ++++ | ++++ | ++++ |
| I-403 | ++++ | ++++ | ++++ | ++++ | ++++ | ++++ |
| I-404 | +++ | ++++ | ++++ | ++++ | ++++ | ++++ |
| I-405 | +++ | +++ | ++++ | +++ | +++ | +++ |
| I-406 | +++ | +++ | +++ | +++ | +++ | +++ |
| I-407 | ++++ | ++++ | ++++ | ++++ | ++++ | ++++ |
| I-408 | +++ | ++++ | ++++ | ++++ | ++++ | ++++ |
| I-409 | ++++ | ++++ | ++++ | ++++ | ++++ | ++++ |
| I-410 | ++++ | ++++ | ++++ | ++++ | ++++ | ++++ |
| I-411 | ++++ | ++++ | ++++ | ++++ | ++++ | ++++ |
| I-412 | ++++ | ++++ | ++++ | ++++ | ++++ | ++++ |
| I-413 | +++ | +++ | +++ | +++ | +++ | +++ |
| I-414 | +++ | +++ | ++++ | ++++ | ++++ | ++++ |
| I-415 | ++++ | ++++ | ++++ | ++++ | ++++ | ++++ |
| II-1-1 | ++ | ++ | ++ | ++ | +++ | ++ |
| II-1-2 | +++ | +++ | +++ | +++ | ++++ | +++ |
| II-1-3 | +++ | +++ | +++ | +++ | ++++ | ++++ |
| II-1-4 | ++ | ++ | ++ | ++ | ++ | ++ |
| II-1-6 | ++ | ++ | ++ | +++ | +++ | +++ |
| II-1-8 | ++ | ++ | +++ | +++ | +++ | +++ |
| II-1-9 | ++ | ++ | +++ | +++ | ++++ | +++ |
| II-1-10 | ++ | +++ | +++ | ++ | +++ | +++ |
| II-1-11 | ++ | ++ | +++ | ++ | ++ | ++ |
| II-1-12 | ++ | ++ | ++ | +++ | +++ | +++ |
| II-1-13 | ++ | ++ | +++ | +++ | ++++ | +++ |
| II-1-14 | ++ | ++ | ++ | +++ | +++ | ++ |
| II-1-15 | ++ | ++ | ++ | ++ | +++ | +++ |
| II-1-16 | ++ | +++ | +++ | +++ | ++++ | +++ |
| II-1-17 | +++ | +++ | +++ | +++ | +++ | +++ |
| II-1-19 | ++ | ++ | ++ | +++ | ++ | ++ |
| II-1-20 | ++ | +++ | ++ | +++ | +++ | ++ |
| II-1-21 | +++ | +++ | +++ | ++ | +++ | +++ |
| II-1-22 | +++ | ++ | +++ | +++ | +++ | +++ |
| II-1-23 | +++ | +++ | +++ | +++ | ++++ | +++ |
| II-1-24 | ++ | ++ | ++ | ++ | ++ | ++ |
| II-1-25 | +++ | +++ | +++ | +++ | ++++ | +++ |
| II-1-26 | +++ | +++ | ++++ | ++++ | ++++ | ++++ |
| II-1-27 | ++ | ++ | ++ | ++ | ++ | ++ |
| II-1-28 | ++ | ++ | ++ | ++ | ++ | ++ |
| II-1-29 | ++ | ++ | ++ | ++ | +++ | ++ |
| II-1-31 | ++ | ++ | ++ | ++ | +++ | ++ |
| II-1-32 | ++ | ++ | ++ | +++ | +++ | +++ |
| II-1-33 | ++ | ++ | ++ | ++ | ++ | ++ |
| II-1-34 | ++ | ++ | +++ | ++ | +++ | ++ |
| II-1-35 | ++ | ++ | ++ | ++ | ++ | ++ |
| II-1-36 | ++ | ++ | ++ | ++ | ++ | ++ |
| II-1-39 | ++ | ++ | +++ | +++ | ++++ | +++ |
| II-1-40 | ++ | ++ | +++ | ++ | ++ | ++ |
| II-1-41 | ++ | +++ | ++ | ++ | +++ | +++ |
| II-1-42 | +++ | +++ | +++ | +++ | +++ | +++ |
| II-1-43 | ++ | +++ | ++ | +++ | +++ | +++ |
| II-1-44 | ++ | ++ | +++ | ++ | ++ | ++ |
| II-1-45 | ++ | +++ | +++ | +++ | ++++ | +++ |
| II-2-1 | ++ | ++ | ++ | ++ | +++ | ++ |
| II-2-4 | ++ | ++ | ++ | ++ | ++ | ++ |
| II-2-5 | +++ | +++ | ++++ | ++++ | ++++ | ++++ |
| II-2-7 | +++ | +++ | ++++ | ++++ | ++++ | ++++ |
| II-2-8 | +++ | +++ | ++++ | +++ | ++++ | ++++ |
| II-2-9 | +++ | +++ | ++++ | ++++ | ++++ | ++++ |
| II-2-12 | +++ | +++ | ++++ | ++++ | ++++ | ++++ |
| II-2-17 | ++ | ++ | +++ | +++ | ++++ | +++ |
| II-2-22 | +++ | ++ | +++ | ++++ | ++++ | +++ |
| II-2-23 | +++ | +++ | +++ | +++ | ++++ | ++++ |

Note: “++++” denotes IC₅₀ ≤ 50 nM; “+++” denotes 50 nM < IC₅₀ ≤ 500 nM; “++” denotes 500 nM < IC₅₀ ≤ 2500 nM.

The other compounds also have good inhibitory activities on the above cell lines stably transfected with kinase.

1. A compound having the general formula: 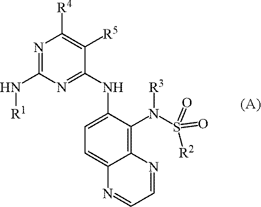
or a stereoisomer, a prodrug, a pharmaceutically acceptable salt or a pharmaceutically acceptable solvate thereof, wherein:
R¹ is selected from:
1) H, C₁—C₆ alkyl, C₁—C₆ fluorine-containing alkyl, C₃—C₈ cycloalkyl, C₁—C₆ heteroatom-containing alkyl, C₃—C₈ heteroatom-containing cycloalkyl;
2) 2-N,N-dimethylaminoethyl, 3-N,N-dimethylaminopropyl, 2-N,N-diethylaminoethyl, 3-N,N-diethylaminopropyl, 2-(N-methylpiperazinyl)ethyl, 2-(N-acetylpiperazinyl)ethyl, 2-morpholinylethyl, 2-thiomorpholinylethyl, 2-piperidylethyl, 2-hydroxyethyl, 3-hydroxypropyl, 2-methoxyethyl, N-methyl-4-piperidyl, N-ethyl-4-piperidyl, N-propyl-4-piperidyl, N-isopropyl-4-piperidyl, N-hydroxyethyl-4-piperidyl, N-cyanomethyl-4-piperidyl, N-(2-cyanoethyl)-4-piperidyl, N-acetyl-4-piperidyl, N-propionyl-4-piperidyl, N-isopropionyl-4-piperidyl, N-cyclopropylformyl-4-piperidyl, N-methylsulfonyl-4-piperidyl, N-ethylsulfonyl-4-piperidyl, N-propylsulfonyl-4-piperidyl, N-isopropylsulfonyl-4-piperidyl, N-cyclopropylsulfonyl-4-piperidyl, 3-N,N-dimethylaminocyclopentyl, 3-N,N-diethylaminocyclopentyl, 3-N,N-dipropylaminocyclopentyl, 3-N,N-isopropylaminocyclopentyl, 4-aminocyclohexyl, 4-N,N-dimethylaminocyclohexyl, 4-N,N-diethylaminocyclohexyl, 4-N,N-dipropylaminocyclohexyl, 4-N,N-diisopropylaminocyclohexyl, 4-acetylaminocyclohexyl, 4-propionylaminocyclohexyl, 4-isopropylformylaminocyclohexyl, 4-methylsulfonylaminocyclohexyl, 4-ethylsulfonylaminocyclohexyl, 4-propylsulfonylaminocyclohexyl, 4-isopropylsulfonylaminocyclohexyl, 4-cyclopropylsulfonylaminocyclohexyl, 4-hydroxycyclohexyl, 4-methoxycyclohexyl, 4-ethoxycyclohexyl, 4-isopropoxycyclohexyl;
3) 1-methyl-3-pyrrolyl, 1-ethyl-3-pyrrolyl, 1-isopropyl-3-pyrrolyl, 1-cyclopropyl-3-pyrrolyl, 1-methyl-3-pyrazolyl, 1-ethyl-3-pyrazolyl, 1-isopropyl-3-pyrazolyl, 1-cyclopropyl-3-pyrazolyl, 1-hydroxyethyl-3-pyrazolyl, 1,5-dimethyl-3-pyrazolyl, 1,5-diethyl-3-pyrazolyl, 1,5-diisopropyl-3-pyrazolyl, 1-methyl-4-pyrazolyl, 1-ethyl-4-pyrazolyl, 1-isopropyl-4-pyrazolyl, 1-cyclopropyl-4-pyrazolyl, 1-hydroxyethyl-4-pyrazolyl, 1-(N-methyl-4-piperidyl)-4-pyrazolyl, 1-(N-t-butoxycarbonyl-4-piperidyl)-4-pyrazolyl, 1,3-dimethyl-4-pyrazolyl, 1-methyl-3-ethyl-4-pyrazolyl, 1-methyl-3-isopropyl-4-pyrazolyl, 1-ethyl-3-methyl-4-pyrazolyl, 1,3-diethyl-4-pyrazolyl, 1-ethyl-3-isopropyl-4-pyrazolyl, 1-isopropyl-3-methyl-4-pyrazolyl, 1-isopropyl-3-ethyl-4-pyrazolyl, 1,3-diisopropyl-4-pyrazolyl, 1,3-dimethyl-5-pyrazolyl, 1-methyl-4-imidazolyl, 3-methyl-5-isoxazolyl;
4) 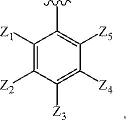
wherein Z
₁, Z₂, Z₃, Z₄, Z₅ each are independently selected from the following groups, and Z₁, Z₂, Z₃, Z₄, Z₅ are not hydrogen at the same time:
(1) H, F, Cl, Br, I, nitro, cyano,
(2) C₁—C₆ alkyl, C₁—C₆ alkoxy, C₁—C₆ oxygen-containing alkyl, C₁—C₆ fluorine-containing alkyl, C₁—C₆ fluorine-containing alkoxy, C₃—C₆ cycloalkyl, C₂—C₆ alkenyl,
(3) 2-N,N-dimethylaminoethoxy, 3-N,N-dimethylaminopropoxy, 2-(N-methylpiperazinyl)ethoxy, 2-(N-acetylpiperazinyl)ethoxy, 2-morpholinylethoxy, 2-thiomorpholinylethoxy, 2-piperidylethoxy, 3-N,N-dimethylaminopropoxy, 3-N,N-diethylaminopropoxy, 3-N,N-diisopropylaminopropoxy, 3-(N-methylpiperazinyl)propoxy, 3-(N-acetylpiperazinyl)propoxy, 3-morpholinylpropoxy, 3-thiomorpholinylpropoxy, 3-piperidylpropoxy, 2-pyridylmethoxy, 3-pyridylmethoxy, 4-pyridylmethoxy, phenylmethoxy, monohalo-substituted phenylmethoxy, gem-dihalo-substituted phenylmethoxy, hetero-dihalo-substituted phenylmethoxy,
(4) piperazinyl, N-methylpiperazinyl, N-ethylpiperazinyl, N-n-propylpiperazinyl, N-isopropylpiperazinyl, 4-(piperidyl-4-)piperazinyl, 4-(N-methylpiperidyl-4-)piperazinyl, 4-(N-ethylpiperidyl-4-)piperazinyl, 4-(N-n-propylpiperidyl-4-)piperazinyl, 4-(N-isopropylpiperidyl-4-)piperazinyl, 4-(azetidin-3-yl)piperazinyl, 4-(N-methyl-azetidin-3-yl)piperazinyl, 4-(N-ethyl-azetidin-3-yl)piperazinyl, 4-(N-n-propyl-azetidin-3-yl)piperazinyl, 4-(N-isopropyl-azetidin-3-yl)piperazinyl, N-cyclopropylpiperazinyl, N-(2-hydroxyethyl)piperazinyl, N-(2-N,N-dimethylaminoethyl)piperazinyl, N-(2-N,N-diethylaminoethyl)piperazinyl, N-(2-cyanoethyl)piperazinyl, N-cyanomethylpiperazinyl, N-(3-hydroxypropyl)piperazinyl, N-(3-N,N-dimethylaminopropyl)piperazinyl, N-(3-N,N-diethylaminopropyl)piperazinyl, N-(N-methyl-4-piperidyl)piperazinyl, N-(N-ethyl-4-piperidyl)piperazinyl, N-t-butoxycarbonylpiperazinyl, N-acetylpiperazinyl, N-propionylpiperazinyl, N-isobutyrylpiperazinyl, N-cyclopropylformylpiperazinyl, N-methylsulfonylpiperazinyl, N-ethylsulfonylpiperazinyl, N-n-propylsulfonylpiperazinyl, N-isopropylsulfonylpiperazinyl, N-cyclopropylsulfonylpiperazinyl, 2-oxo-piperazin-4-yl,
(5) morpholinyl, 2,6-dimethylmorpholinyl, thiomorpholinyl,
(6) tetrahydropyrrolyl, 3-(N,N-dimethylamino)tetrahydropyrrolyl, 3-(N,N-diethylamino)tetrahydropyrrolyl,
(7) imidazolyl, 4-methyl-1-imidazolyl,
(8) piperidyl, 4-N,N-dimethylaminopiperidyl, 4-N,N-diethylaminopiperidyl, 4-N,N-diisopropylaminopiperidyl, 4-hydroxypiperidyl, 4-cyanopiperidyl, 4-methoxypiperidyl, 4-ethoxypiperidyl, 4-(piperazinyl-1-)piperidyl, 4-(N-methylpiperazinyl-1-)piperidyl, 4-(N-ethylpiperazinyl-1-)piperidyl, 4-(N-isopropylpiperazinyl-1-)piperidyl, 4-(N-acetylpiperazinyl-1-)piperidyl, 4-(N-t-butoxycarbonylpiperazinyl-1-)piperidyl, 4-(N-methylsulfonylpiperazinyl-1-)piperidyl, 4-(N-(2-hydroxyethyl)piperazinyl-1-)piperidyl, 4-(N-(2-cyanoethyl)piperazinyl-1-)piperidyl, 4-(N-(3-hydroxypropyl)piperazinyl-1-)piperidyl, 4-(N-(2-N,N-dimethylaminoethyl)piperazinyl-1-)piperidyl, 4-(N-(2-N,N-diethylethyl)piperazinyl-1-)piperidyl, 4-(N-(3-N,N-dimethylpropyl)piperazinyl-1-)piperidyl, 4-(N-(3-N,N-diethylpropyl)piperazinyl-1-)piperidyl, 4-(tetrahydropyrrolyl-1-)piperidyl, 4-(3-N,N-dimethylaminotetrahydropyrrolyl-1-)piperidyl, 4-(3-N,N-diethylaminotetrahydropyrrolyl-1-)piperidyl,
(9) piperidin-4-yl, N-methyl-4-piperidyl, N-ethyl-4-piperidyl, N-isopropyl-4-piperidyl, N-acetyl-4-piperidyl, N-t-butoxycarbonyl-4-piperidyl, N-methylsulfonyl-4-piperidyl, N-(2-hydroxyethyl)-4-piperidyl, N-(2-N,N-dimethylaminoethyl)-4-piperidyl, N-(2-N,N-diethylaminoethyl)-4-piperidyl, N-(2-cyanoethyl)-4-piperidyl, N-(3-hydroxypropyl)-4-piperidyl, N-(3-N,N-dimethylaminopropyl)-4-piperidyl, N-(3-N,N-diethylaminopropyl)-4-piperidyl, N-(3-cyanopropyl)-4-piperidyl, N-cyanomethylene-4-piperidyl,
(10) amino, N,N-dimethylamino, N,N-diethylamino, N,N-di-n-propylamino, N,N-diisopropylamino, 2-hydroxyethylamino, 2-N,N-dimethylaminoethylamino, 2-morpholinylethylamino, 2-(N-methylpiperazinyl)ethylamino, 3-N,N-dimethylaminopropylamino, 3-N,N-diethylaminopropylamino, 3-N,N-diisopropylaminopropylamino, 3-morpholinylpropylamino, 3-(N-methylpiperazinyl)propylamino, N-methylpiperidyl-4-amino, N-ethylpiperidyl-4-amino, N-n-propylpiperidyl-4-amino, N-isopropylpiperidyl-4-amino, acetamido, propionamido, 2-(N,N-dimethylamino)acetamido, 2-hydroxyacetamido, 2-methoxyacetamido, methylsulfamido, ethylsulfamido, n-propylsulfamido, isopropylsulfamido, cyclopropylsulfamido, N-methyl-N-(N-methyl-3-tetrahydropyrrolyl)amino, N-(N-methyl-3-tetrahydropyrrolyl)amino, N-methyl-N-(2-N,N-dimethylamino)ethylamino,
(11) aminomethylene, N,N-dimethylaminomethylene, N,N-diethylaminomethylene, 4-methyl-1-piperazinylmethylene, 4-ethyl-1-piperazinylmethylene, 4-propyl-1-piperazinylmethylene, 4-isopropyl-1-piperazinylmethylene, 4-(2-hydroxyethyl)-1-piperazinylmethylene, 4-(2-N,N-dimethylaminoethyl)-1-piperazinylmethylene, piperidyl-1-methylene, 4-(N,N-dimethylamino)-1-piperidylmethylene, 4-hydroxypiperidyl-1-methylene, tetrahydropyrrolyl-1-methylene, 3-(N,N-dimethylamino)-1-tetrahydropyrrolylmethylene, benzyl, cyclopropylmethylene, cyclobutylmethylene, cyclopentylmethylene, cyclohexylmethylene,
(12) aminoformyl, methylaminoformyl, N,N-dimethylaminoformyl, ethylaminoformyl, cyclopropylaminoformyl, cyclobutylaminoformyl, cyclopentylaminoformyl, cyclohexylaminoformyl, aminoacetyl, methylaminoacetyl, 2-(N,N-dimethylamino)acetyl, ethylaminoacetyl, cyclopropylaminoacetyl, cyclobutylaminoacetyl, cyclopentylaminoacetyl, cyclohexylaminoacetyl, tetrahydropyrrolyl-1-formyl, 3-(N,N-dimethylamino)tetrahydropyrrolyl-1-formyl, 3-(N,N-diethylamino)tetrahydropyrrolyl-1-formyl, morpholinyl-4-formyl, thiomorpholinyl-4-formyl, 2,6-dimethylmorpholinyl-4-formyl, piperidyl-1-formyl, 4-hydroxypiperidyl-1-formyl, 4-(N,N-dimethylamino)piperidyl-1-formyl, 4-(N,N-diethylamino)piperidyl-1-formyl, 4-(N-methyl-1-piperazinyl)piperidyl-1-formyl, 4-(N-ethyl-1-piperazinyl)piperidyl-1-formyl, N-methylpiperazinyl-1-formyl, N-ethylpiperazinyl-1-formyl, N-isopropylpiperazinyl-1-formyl, N-(2-hydroxyethyl)piperazinyl-1-formyl, N-(2-N,N-dimethylaminoethyl)piperazinyl-1-formyl, N-(2-N,N-diethylaminoethyl)piperazinyl-1-formyl, 4-(N-methyl-4-piperidyl)piperazinyl-1-formyl, 4-(N-ethyl-4-piperidyl)piperazinyl-1-formyl,
(13) hydroxyformyl, methoxyformyl, ethoxyformyl, propoxyformyl, isopropoxyformyl, n-butoxyformyl, iso-butoxyformyl, t-butoxyformyl,
(14) hydroxysulfonyl, aminosulfonyl, methylaminosulfonyl, ethylaminosulfonyl, propylaminosulfonyl, isopropylaminosulfonyl, cyclopropylaminosulfonyl, cyclobutylaminosulfonyl, cyclopentylaminosulfonyl, cyclohexylaminosulfonyl, tetrahydropyrrolyl-1-sulfonyl, 3-(N,N-dimethylamino)tetrahydropyrrolyl-1-sulfonyl, 3-(N,N-diethylamino)tetrahydropyrrolyl-1-sulfonyl, morpholinyl-4-sulfonyl, thiomorpholinyl-4-sulfonyl, 2,6-dimethylmorpholinyl-4-sulfonyl, piperidyl-1-sulfonyl, 4-hydroxypiperidyl-1-sulfonyl, 4-(N,N-dimethylamino)piperidyl-1-sulfonyl, 4-(N,N-diethylamino)piperidyl-1-sulfonyl, 4-(N-methyl-1-piperazinyl)piperidyl-1-sulfonyl, 4-(N-ethyl-1-piperazinyl)piperidyl-1-sulfonyl, N-methylpiperazinyl-1-sulfonyl, N-ethylpiperazinyl-1-sulfonyl, N-isopropylpiperazinyl-1-sulfonyl, N-(2-hydroxyethyl)piperazinyl-1-sulfonyl, N-(2-N,N-dimethylaminoethyl)piperazinyl-1-sulfonyl, N-(2-N,N-diethylaminoethyl)piperazinyl-1-sulfonyl, 4-(N-methyl-4-piperidyl)piperazinyl-1-sulfonyl, 4-(N-ethyl-4-piperidyl)piperazinyl-1-sulfonyl,
(15) aminoformamido, methylaminoformamido, ethylaminoformamido, propylaminoformamido, isopropylaminoformamido, cyclopropylaminoformamido, cyclobutylaminoformamido, cyclopentylaminoformamido, piperidyl-1-formamido, 4-hydroxypiperidyl-1-formamido, 4-N,N-dimethylpiperidyl-1-formamido, 4-N,N-diethylpiperidyl-1-formamido, tetrahydropyrrolyl-1-formamido, 3-N,N-dimethyltetrahydropyrrolyl-1-formamido, 3-N,N-diethyltetrahydropyrrolyl-1-formamido, N-methylpiperazinyl-1-formamido, N-ethylpiperazinyl-1-formamido, N-acetylpiperazinyl-1-formamido, N-t-butoxycarbonylpiperazinyl-1-formamido, N-(2-hydroxyethyl)piperazinyl-1-formamido, N-(2-cyanoethyl)piperazinyl-1-formamido, N-(2-N,N-dimethylaminoethyl)piperazinyl-1-formamido, N-(2-N,N-diethylaminoethyl)piperazinyl-1-formamido, N-(3-hydroxypropyl)piperazinyl-1-formamido, N-(3-N,N-dimethylpropyl)piperazinyl-1-formamido, N-(3-N,N-diethylpropyl)piperazinyl-1-formamido, morpholinyl-1-formamido, 3,5-dimethylmorpholinyl-1-formamido, 4-(tetrahydropyrrolyl)piperidyl-1-formamido, 4-(N-methyl-1-piperazinyl)piperidyl-1-formamido, 4-(N-ethyl-1-piperazinyl)piperidyl-1-formamido, 4-(N-acetyl-1-piperazinyl)piperidyl-1-formamido, N-(N-methyl-4-piperidyl)piperazinyl-1-formamido,
(16) Z₃ and Z₄ may form an oxygen-containing substituted or unsubstituted 5- to 7-membered ring; the substituent may be selected from the same substituents as described above for Z₁,
(17) Z₃ and Z₄ may form a nitrogen-containing substituted or unsubstituted 5- to 7-membered ring; the substituent may be selected from the same substituents as described above for Z₁,
(18) 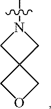 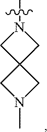 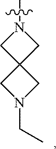 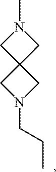 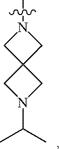 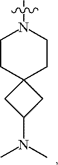 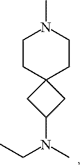 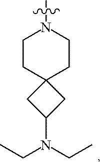 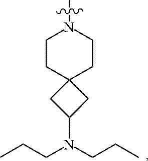 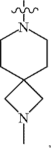 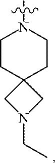 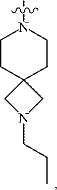 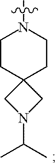
5) 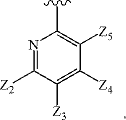
wherein Z
₂, Z₃, Z₄, Z₅ are the same as defined in 4), and Z₂, Z₃, Z₄, Z₅ are not hydrogen at the same time;
6) 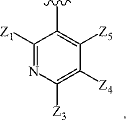
wherein Z
₁, Z₃, Z₄, Z₅ are the same as defined in 4), and Z₁, Z₃, Z₄, Z₅ are not hydrogen at the same time;
7) 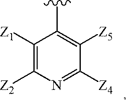
wherein Z
₁, Z₂, Z₄, Z₅ are the same as defined in 4), and Z₁, Z₂, Z₄, Z₅ may be hydrogen at the same time;
8) 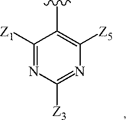
wherein Z
₁, Z₃, Z₅ are the same as defined in 4), and Z₁, Z₃, Z₅ are not hydrogen at the same time;
preferably, R¹ is selected from:
1) H, C₁—C₆ alkyl, C₁—C₆ fluorine-containing alkyl, C₃—C₈ cycloalkyl, C₁—C₆ heteroatom-containing alkyl, C₃—C₈ heteroatom-containing cycloalkyl;
2) 2-N,N-dimethylaminoethyl, 3-N,N-dimethylaminopropyl, 2-N,N-diethylaminoethyl, 3-N,N-diethylaminopropyl, 2-(N-methylpiperazinyl)ethyl, 2-(N-acetylpiperazinyl)ethyl, 2-morpholinylethyl, 2-thiomorpholinylethyl, 2-piperidylethyl, 2-hydroxyethyl, 3-hydroxypropyl, 2-methoxyethyl, N-methyl-4-piperidyl, N-ethyl-4-piperidyl, N-propyl-4-piperidyl, N-isopropyl-4-piperidyl, N-hydroxyethyl-4-piperidyl, N-cyanomethyl-4-piperidyl, N-(2-cyanoethyl)-4-piperidyl, 3-N,N-dimethylaminocyclopentyl, 3-N,N-diethylaminocyclopentyl, 3-N,N-dipropylaminocyclopentyl, 3-N,N-isopropylaminocyclopentyl, 4-aminocyclohexyl, 4-N,N-dimethylaminocyclohexyl, 4-N,N-diethylaminocyclohexyl, 4-N,N-dipropylaminocyclohexyl, 4-N,N-diisopropylaminocyclohexyl, 4-hydroxycyclohexyl, 4-methoxycyclohexyl, 4-ethoxycyclohexyl, 4-isopropoxycyclohexyl;
3) 1-methyl-3-pyrrolyl, 1-ethyl-3-pyrrolyl, 1-isopropyl-3-pyrrolyl, 1-cyclopropyl-3-pyrrolyl, 1-methyl-3-pyrazolyl, 1-ethyl-3-pyrazolyl, 1-isopropyl-3-pyrazolyl, 1-cyclopropyl-3-pyrazolyl, 1-hydroxyethyl-3-pyrazolyl, 1,5-dimethyl-3-pyrazolyl, 1,5-diethyl-3-pyrazolyl, 1,5-diisopropyl-3-pyrazolyl, 1-methyl-4-pyrazolyl, 1-ethyl-4-pyrazolyl, 1-isopropyl-4-pyrazolyl, 1-cyclopropyl-4-pyrazolyl, 1-hydroxyethyl-4-pyrazolyl, 1,3-dimethyl-4-pyrazolyl, 1-methyl-3-ethyl-4-pyrazolyl, 1-methyl-3-isopropyl-4-pyrazolyl, 1-ethyl-3-methyl-4-pyrazolyl, 1,3-diethyl-4-pyrazolyl, 1-ethyl-3-isopropyl-4-pyrazolyl, 1-isopropyl-3-methyl-4-pyrazolyl, 1-isopropyl-3-ethyl-4-pyrazolyl, 1,3-diisopropyl-4-pyrazolyl, 1,3-dimethyl-5-pyrazolyl, 1-methyl-4-imidazolyl, 3-methyl-5-isoxazolyl;
4) 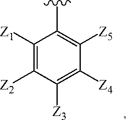
wherein Z
₁, Z₂, Z₃, Z₄, Z₅ each are independently selected from the following groups, and Z₁, Z₂, Z₃, Z₄, Z₅ are not hydrogen at the same time:
(1) H, F, Cl, Br, I, nitro, cyano,
(2) C₁—C₆ alkyl, C₁—C₆ alkoxy, C₁—C₆ oxygen-containing alkyl, C₁—C₆ fluorine-containing alkyl, C₁—C₆ fluorine-containing alkoxy, C₃—C₆ cycloalkyl, C₂—C₆ alkenyl,
(3) piperazinyl, N-methylpiperazinyl, N-ethylpiperazinyl, N-n-propylpiperazinyl, N-isopropylpiperazinyl, 4-(piperidyl-4-)piperazinyl, 4-(N-methylpiperidyl-4-)piperazinyl, 4-(N-ethylpiperidyl-4-)piperazinyl, 4-(N-n-propylpiperidyl-4-)piperazinyl, 4-(N-isopropylpiperidyl-4-)piperazinyl, 4-(azetidin-3-yl)piperazinyl, 4-(N-methyl-azetidin-3-yl)piperazinyl, 4-(N-ethyl-azetidin-3-yl)piperazinyl, 4-(N-n-propyl-azetidin-3-yl)piperazinyl, 4-(N-isopropyl-azetidin-3-yl)piperazinyl, N-cyclopropylpiperazinyl, N-(2-hydroxyethyl)piperazinyl, N-(2-N,N-dimethylaminoethyl)piperazinyl, N-(2-N,N-diethylaminoethyl)piperazinyl, N-(2-cyanoethyl)piperazinyl, N-cyanomethylpiperazinyl, N-(3-hydroxypropyl)piperazinyl, N-(3-N,N-dimethylaminopropyl)piperazinyl, N-(3-N,N-diethylaminopropyl)piperazinyl,
(4) morpholinyl, 2,6-dimethylmorpholinyl, thiomorpholinyl,
(5) tetrahydropyrrolyl, 3-(N,N-dimethylamino)tetrahydropyrrolyl, 3-(N,N-diethylamino)tetrahydropyrrolyl,
(6) imidazolyl, 4-methyl-1-imidazolyl,
(7) piperidyl, 4-N,N-dimethylaminopiperidyl, 4-N,N-diethylaminopiperidyl, 4-N,N-diisopropylaminopiperidyl, 4-hydroxypiperidyl, 4-cyanopiperidyl, 4-methoxypiperidyl, 4-ethoxypiperidyl, 4-(piperazinyl-1-)piperidyl, 4-(N-methylpiperazinyl-1-)piperidyl, 4-(N-ethylpiperazinyl-1-)piperidyl, 4-(N-isopropylpiperazinyl-1-)piperidyl, 4-(N-(2-hydroxyethyl)piperazinyl-1-)piperidyl, 4-(N-(2-cyanoethyl)piperazinyl-1-)piperidyl, 4-(N-(3-hydroxypropyl)piperazinyl-1-)piperidyl, 4-(N-(2-N,N-dimethylaminoethyl)piperazinyl-1-)piperidyl, 4-(N-(2-N,N-diethylethyl)piperazinyl-1-)piperidyl, 4-(N-(3-N,N-dimethylpropyl)piperazinyl-1-)piperidyl, 4-(N-(3-N,N-diethylpropyl)piperazinyl-1-)piperidyl, 4-(tetrahydropyrrolyl-1-)piperidyl, 4-(3-N,N-dimethylaminotetrahydropyrrolyl-1-)piperidyl, 4-(3-N,N-diethylaminotetrahydropyrrolyl-1-)piperidyl,
(8) piperidin-4-yl, N-methyl-4-piperidyl, N-ethyl-4-piperidyl, N-isopropyl-4-piperidyl, N-(2-hydroxyethyl)-4-piperidyl, N-(2-N,N-dimethylaminoethyl)-4-piperidyl, N-(2-N,N-diethylaminoethyl)-4-piperidyl, N-(2-cyanoethyl)-4-piperidyl, N-(3-hydroxypropyl)-4-piperidyl, N-(3-N,N-dimethylaminopropyl)-4-piperidyl, N-(3-N,N-diethylaminopropyl)-4-piperidyl, N-(3-cyanopropyl)-4-piperidyl, N-cyanomethylene-4-piperidyl,
(9) amino, N,N-dimethylamino, N,N-diethylamino, N,N-di-n-propylamino, N,N-diisopropylamino, 2-hydroxyethylamino, 2-N,N-dimethylaminoethylamino, 3-N,N-dimethylam inopropylamino, 3-N,N-diethylaminopropylamino, 3-N,N-diisopropylaminopropylamino, N-methylpiperidyl-4-amino, N-ethylpiperidyl-4-amino, N-n-propylpiperidyl-4-amino, N-isopropylpiperidyl-4-amino, acetamido, propionamido, 2-(N,N-dimethylamino)acetamido, 2-hydroxyacetamido, 2-methoxyacetamido, methylsulfamido, ethylsulfamido, n-propylsulfamido, isopropylsulfamido, cyclopropylsulfamido, N-methyl-N-(N-methyl-3-tetrahydropyrrolyl)amino, N-(N-methyl-3-tetrahydropyrrolyl)amino, N-methyl-N-(2-N,N-dimethylamino)ethylamino,
(10) 4-methyl-1-piperazinylmethylene, 4-ethyl-1-piperazinylmethylene, 4-propyl-1-piperazinylmethylene, 4-isopropyl-1-piperazinylmethylene, 4-(2-hydroxyethyl)-1-piperazinylmethylene, 4-(2-N,N-dimethylam inoethyl)-1-piperazinylmethylene, piperidyl-1-methylene, 4-(N,N-dimethylamino)-1-piperidylmethylene, 4-hydroxypiperidyl-1-methylene, tetrahydropyrrolyl-1-methylene, 3-(N,N-dimethylamino)-1-tetrahydropyrrolylmethylene, benzyl, cyclopropylmethylene, cyclobutylmethylene, cyclopentylmethylene, cyclohexylmethylene,
(11) aminoformyl, methylaminoformyl, N,N-dimethylaminoformyl, ethylaminoformyl, aminoacetyl, methylaminoacetyl, 2-(N,N-dimethylamino)acetyl, ethylaminoacetyl, tetrahydropyrrolyl-1-formyl, 3-(N,N-dimethylam ino)tetrahydropyrrolyl-1-formyl, 3-(N,N-diethylamino)tetrahydropyrrolyl-1-formyl, morpholinyl-4-formyl, thiomorpholinyl-4-formyl, 2,6-dimethylmorpholinyl-4-formyl, piperidyl-1-formyl, 4-hydroxypiperidyl-1-formyl, 4-(N,N-dimethylamino)piperidyl-1-formyl, 4-(N,N-diethylamino)piperidyl-1-formyl, 4-(N-methyl-1-piperazinyl)piperidyl-1-formyl, 4-(N-ethyl-1-piperazinyl)piperidyl-1-formyl, N-methylpiperazinyl-1-formyl, N-ethylpiperazinyl-1-formyl, N-isopropylpiperazinyl-1-formyl, N-(2-hydroxyethyl)piperazinyl-1-formyl, N-(2-N,N-dimethylam inoethyl)piperazinyl-1-formyl, N-(2-N,N-diethylaminoethyl)piperazinyl-1-formyl, 4-(N-methyl-4-piperidyl)piperazinyl-1-formyl, 4-(N-ethyl-4-piperidyl)piperazinyl-1-formyl,
(12) hydroxysulfonyl, aminosulfonyl, methylaminosulfonyl, ethylaminosulfonyl, propylaminosulfonyl, isopropylaminosulfonyl, cyclopropylaminosulfonyl, cyclobutylaminosulfonyl, cyclopentylaminosulfonyl, cyclohexylaminosulfonyl, tetrahydropyrrolyl-1-sulfonyl, 3-(N,N-dimethylamino)tetrahydropyrrolyl-1-sulfonyl, 3-(N,N-diethylamino)tetrahydropyrrolyl-1-sulfonyl, morpholinyl-4-sulfonyl, thiomorpholinyl-4-sulfonyl, 2,6-dimethylmorpholinyl-4-sulfonyl, piperidyl-1-sulfonyl, 4-hydroxypiperidyl-1-sulfonyl, 4-(N,N-dimethylamino)piperidyl-1-sulfonyl, 4-(N,N-diethylamino)piperidyl-1-sulfonyl, N-methylpiperazinyl-1-sulfonyl, N-ethylpiperazinyl-1-sulfonyl, N-isopropylpiperazinyl-1-sulfonyl, N-(2-hydroxyethyl)piperazinyl-1-sulfonyl, N-(2-N,N-dimethylam inoethyl)piperazinyl-1-sulfonyl, N-(2-N,N-diethylaminoethyl)piperazinyl-1-sulfonyl,
(13) aminoformamido, methylaminoformamido, ethylaminoformamido, propylaminoformamido, isopropylaminoformamido,
(14) 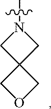 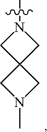 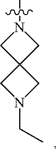 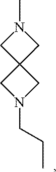 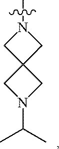 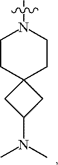 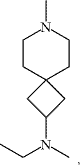 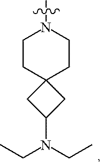 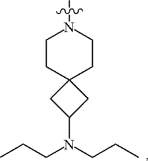 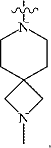 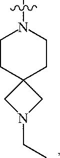 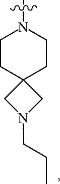 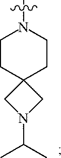
5) 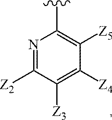
wherein Z
₂, Z₃, Z₄, Z₅ are the same as defined in 4), and Z₂, Z₃, Z₄, Z₅ are not hydrogen at the same time;
6) 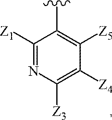
wherein Z
₁, Z₃, Z₄, Z₅ are the same as defined in 4), and Z₁, Z₃, Z₄, Z₅ are not hydrogen at the same time;
7) 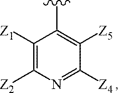
wherein Z
₁, Z₂, Z₄, Z₅ are the same as defined in 4), and Z₁, Z₂, Z₄, Z₅ may be hydrogen at the same time;
8) 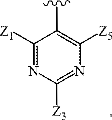
wherein Z
₁, Z₃, Z₅ are the same as defined in 4), and Z₁, Z₃, Z₅ are not hydrogen at the same time;
9) 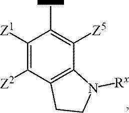
wherein Z
₁, Z₂, Z₅ are the same as defined in 4), and Z₁, Z₂, Z₅ may be hydrogen at the same time, R^{x} is selected from the same substituents as described above for Z¹;
10) 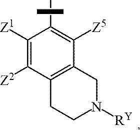
wherein Z
₁, Z₂, Z₅ are the same as defined in 4), and Z₁, Z₂, Z₅ may be hydrogen at the same time, R^{y} is selected from the same substituents as described above for Z¹;
11) 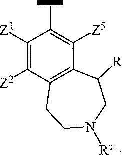
wherein Z
₁, Z₂, Z₅ are the same as defined in 4), and Z₁, Z₂, Z₅ may be hydrogen at the same time, R^{z}, R each are independently selected from the same substituents as described above for Z¹;
more preferably, R¹ is selected from:
1) H, C₁—C₆ alkyl, C₃—C₈ cycloalkyl;
2) 2-N,N-dimethylaminoethyl, 3-N,N-dimethylaminopropyl, 2-N,N-diethylaminoethyl, 3-N,N-diethylaminopropyl, 2-hydroxyethyl, 3-hydroxypropyl, N-methyl-4-piperidyl, N-ethyl-4-piperidyl, N-propyl-4-piperidyl, N-isopropyl-4-piperidyl, 4-aminocyclohexyl, 4-N,N-dimethylaminocyclohexyl, 4-N,N-diethylaminocyclohexyl, 4-N,N-dipropylaminocyclohexyl, 4-N,N-diisopropylaminocyclohexyl;
3) 1-methyl-3-pyrazolyl, 1-ethyl-3-pyrazolyl, 1-isopropyl-3-pyrazolyl, 1-cyclopropyl-3-pyrazolyl, 1,5-dimethyl-3-pyrazolyl, 1,5-diethyl-3-pyrazolyl, 1,5-diisopropyl-3-pyrazolyl, 1-methyl-4-pyrazolyl, 1-ethyl-4-pyrazolyl, 1-isopropyl-4-pyrazolyl, 1-cyclopropyl-4-pyrazolyl, 1,3-dimethyl-4-pyrazolyl, 1-methyl-3-ethyl-4-pyrazolyl, 1-methyl-3-isopropyl-4-pyrazolyl, 1-ethyl-3-methyl-4-pyrazolyl, 1,3-diethyl-4-pyrazolyl, 1-ethyl-3-isopropyl-4-pyrazolyl, 1-isopropyl-3-methyl-4-pyrazolyl, 1-isopropyl-3-ethyl-4-pyrazolyl, 1,3-diisopropyl-4-pyrazolyl, 1,3-dimethyl-5-pyrazolyl, 1-methyl-4-imidazolyl;
4) 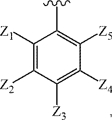
wherein Z
₁, Z₂, Z₃, Z₄, Z₅ each are independently selected from the following groups, and Z₁, Z₂, Z₃, Z₄, Z₅ are not hydrogen at the same time:
(1) H, F, Cl, Br, I, nitro, cyano,
(2) C₁—C₆ alkyl, C₁—C₆ alkoxy, C₁—C₆ fluorine-containing alkyl, C₁—C₆ fluorine-containing alkoxy, C₂—C₆ alkenyl, C₃—C₆ cycloalkyl,
(3) piperazinyl, N-methylpiperazinyl, N-ethylpiperazinyl, N-n-propylpiperazinyl, N-isopropylpiperazinyl, 4-(piperidyl-4-)piperazinyl, 4-(N-methylpiperidyl-4-)piperazinyl, 4-(N-ethylpiperidyl-4-)piperazinyl, 4-(N-n-propylpiperidyl-4-)piperazinyl, 4-(N-isopropylpiperidyl-4-)piperazinyl, 4-(azetidin-3-yl)piperazinyl, 4-(N-methyl-azetidin-3-yl)piperazinyl, 4-(N-ethyl-azetidin-3-yl)piperazinyl, 4-(N-n-propyl-azetidin-3-yl)piperazinyl, 4-(N-isopropyl-azetidin-3-yl)piperazinyl,
(4) morpholinyl, 2,6-dimethylmorpholinyl, thiomorpholinyl,
(5) 3-(N,N-dimethylamino)tetrahydropyrrolyl, 3-(N,N-diethylamino)tetrahydropyrrolyl,
(6) 4-N,N-dimethylaminopiperidyl, 4-N,N-diethylaminopiperidyl, 4-N,N-diisopropylaminopiperidyl, 4-hydroxypiperidyl, 4-cyanopiperidyl, 4-(piperazinyl-1-)piperidyl, 4-(N-methylpiperazinyl-1-)piperidyl, 4-(N-ethylpiperazinyl-1-)piperidyl, 4-(N-isopropylpiperazinyl-1-)piperidyl, 4-(tetrahydropyrrolyl-1-)piperidyl,
(7) piperidin-4-yl, N-methyl-4-piperidyl, N-ethyl-4-piperidyl, N-isopropyl-4-piperidyl,
(8) aminoformamido, methylaminoformamido, ethylaminoformamido, propylaminoformamido, isopropylaminoformamido, 2-(N,N-dimethylamino)acetamido, cyclopropylsulfamido, 2-hydroxyethylamino, 2-N,N-dimethylaminoethylamino, 3-N,N-dimethylaminopropylamino, 3-N,N-diethylaminopropylamino, 3-N,N-diisopropylaminopropylamino, N-methyl-N-(2-N,N-dimethylamino)ethylamino,
(9) 4-methyl-1-piperazinylmethylene, 4-ethyl-1-piperazinylmethylene, 4-propyl-1-piperazinylmethylene, 4-isopropyl-1-piperazinylmethylene, benzyl, cyclopropylmethylene, cyclobutylmethylene, cyclopentylmethylene, cyclohexylmethylene,
(10) 4-(N-methyl-1-piperazinyl)piperidyl-1-formyl, 4-(N-ethyl-1-piperazinyl)piperidyl-1-formyl, N-methylpiperazinyl-1-formyl, N-ethylpiperazinyl-1-formyl, N-isopropylpiperazinyl-1-formyl,
(11) cyclopropylaminosulfonyl, cyclobutylaminosulfonyl, cyclopentylaminosulfonyl, cyclohexylaminosulfonyl, N-methylpiperazinyl-1-sulfonyl, N-ethylpiperazinyl-1-sulfonyl, N-isopropylpiperazinyl-1-sulfonyl,
(12) 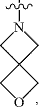 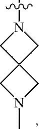 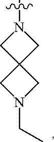 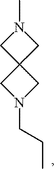 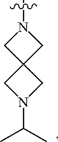 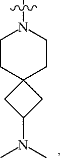 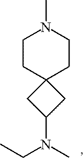 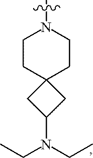 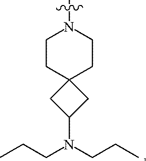 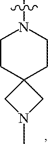 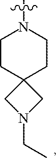 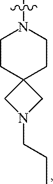 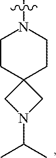
5) 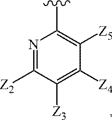
wherein Z
₂, Z₃, Z₄, Z₅ each are independently selected from H, C₁—C₆ alkyl, C₁—C₆ alkoxy, piperazinyl, N-methylpiperazinyl, N-ethylpiperazinyl, N-n-propylpiperazinyl, or N-isopropylpiperazinyl, and Z₂, Z₃, Z₄, Z₅ are not hydrogen at the same time;
6) 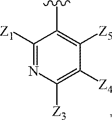
wherein Z
₁, Z₃, Z₄, Z₅ each are independently selected from H, C₁—C₆ alkyl, C₁—C₆ alkoxy, N-(N-methyl-3-tetrahydropyrrolyl)amino, N-methyl-N-(N-methyl-3-tetrahydropyrrolyl)amino, N-methylpiperazinyl, N-ethylpiperazinyl, N-n-propylpiperazinyl, N-isopropylpiperazinyl, 4-(N-methylpiperazinyl-1-)piperidyl, 4-(N-ethylpiperazinyl-1-)piperidyl, or 4-(N-isopropylpiperazinyl-1-)piperidyl, and Z₁, Z₃, Z₄, Z₅ are not hydrogen at the same time;
7) 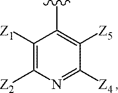
wherein Z
₁, Z₂, Z₄, Z₅ each are independently selected from H, or C₁-C₆ alkyl, and Z₁, Z₂, Z₄, Z₅ may be hydrogen at the same time;
8) 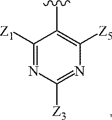
wherein Z
₁, Z₃, Z₅ each are independently selected from H, C₁—C₆ alkyl, C₁—C₆ alkoxy, N-methylpiperazinyl, N-ethylpiperazinyl, N-n-propylpiperazinyl, or N-isopropylpiperazinyl, and Z₁, Z₃, Z₅ are not hydrogen at the same time;
9) 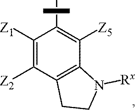
wherein Z
₁, Z₂, Z₅ each are independently selected from H, C₁—C₆ alkyl, or C₁—C₆ alkoxy, and Z₁, Z₂, Z₅ may be hydrogen at the same time, R^{x} is selected from aminoformyl, methylaminoformyl, N,N-dimethylaminoformyl, ethylaminoformyl, aminoacetyl, methylaminoacetyl, 2-(N,N-dimethylamino)acetyl, or ethylaminoacetyl;
10) 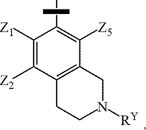
wherein Z
₁, Z₂, Z₅ each are independently selected from H, F, Cl, Br, I, C₁—C₆ alkyl, C₁—C₆ alkoxy, C₁—C₆ fluorine-containing alkyl, or C₁—C₆ fluorine-containing alkoxy, and Z₁, Z₂, Z₅ may be hydrogen at the same time, R^{y} is C₁—C₆ alkyl;
11) 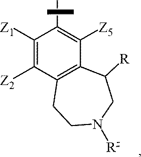
wherein Z
₁, Z₂, Z₅ each are independently selected from H, or C₁—C₆ alkyl, and Z₁, Z₂, Z₅ may be hydrogen at the same time, R^{z}, R each are independently selected from C₁—C₆ alkyl;
most preferably, R¹ is selected from:
1) cyclopropyl, 2-N,N-dimethylaminoethyl, 2-hydroxyethyl, N-methyl-4-piperidyl, 4-aminocyclohexyl, 1-methyl-3-pyrazolyl, 1-ethyl-3-pyrazolyl, 1-isopropyl-3-pyrazolyl, 1,5-dimethyl-3-pyrazolyl, 1-methyl-4-pyrazolyl, 1-ethyl-4-pyrazolyl, 1-isopropyl-4-pyrazolyl, 1-ethyl-3-methyl-4-pyrazolyl, 1,3-dimethyl-5-pyrazolyl, 1-methyl-4-imidazolyl;
2) 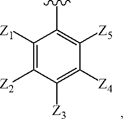
wherein Z
₁, Z₂, Z₃, Z₄, Z₅ each are independently selected from the following groups, and Z₁, Z₂, Z₃, Z₄, Z₅ are not hydrogen at the same time: H, F, 2-hydroxyethylamino, 2-N,N-dimethylaminoethylamino, N-methyl-N-(2-N,N-dimethylamino)ethylamino, methyl, ethyl, isopropyl, cyclopropyl, methoxy, ethoxy, isopropoxy, trifluoromethyl, trifluoromethoxy, vinyl, piperazin-1-yl, N-methylpiperazinyl, 4-(N-methylpiperidyl-4-)piperazinyl, 4-(azetidin-3-yl)piperazinyl, morpholinyl, 3-(N,N-dimethylamino)tetrahydropyrrolyl, 4-N,N-dimethylaminopiperidyl, 4-hydroxypiperidyl, 4-cyanopiperidyl, 4-(N-methylpiperazinyl-1-)piperidyl, 4-(piperazinyl-1-)piperidyl, 4-(tetrahydropyrrolyl-1-)piperidyl, N-methyl-4-piperidyl, 2-(N,N-dimethylamino)acetamido, cyclopropylsulfamido, N-methyl-N-(2-N,N-dimethylamino)ethylamino, 4-methyl-1-piperazinylmethylene, benzyl, cyclopropylmethylene, 4-(N-methyl-1-piperazinyl)piperidyl-1-formyl, N-methylpiperazinyl-1-formyl, cyclopropylaminosulfonyl, N-methylpiperazinyl-1-sulfonyl, 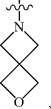 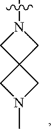 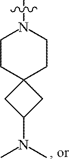 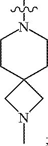
3) 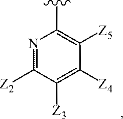
wherein Z
₂, Z₃, Z₄, Z₅ each are independently selected from H, methoxy, or N-methylpiperazinyl, and Z₂, Z₃, Z₄, Z₅ are not hydrogen at the same time;
4) 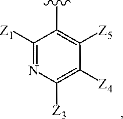
wherein Z
₁, Z₃, Z₄, Z₅ each are independently selected from H, methyl, ethyl, methoxy, N-(N-methyl-3-tetrahydropyrrolyl)amino, N-methyl-N-(N-methyl-3-tetrahydropyrrolyl)amino, N-methylpiperazinyl, or 4-(N-methylpiperazinyl-1-)piperidyl, and Z₁, Z₃, Z₄, Z₅ are not hydrogen at the same time;
5) 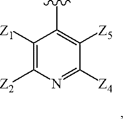
wherein Z
₁, Z₂, Z₄, Z₅ are H;
6) 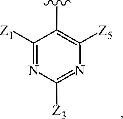
wherein Z
₁, Z₃, Z₅ each are independently selected from H, methoxy, or N-methylpiperazinyl, and Z₁, Z₃, Z₅ are not hydrogen at the same time;
7) 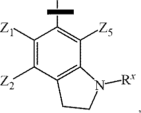
wherein Z
₁, Z₂, Z₅ each are independently selected from H, or methoxy, R^{x} is 2-(N,N-dimethylamino)acetyl;
8) 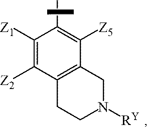
wherein Z
₁, Z₂, Z₅ each are independently selected from H F Cl, methyl, methoxy, trifluoromethyl, or trifluoromethoxy, R^{y} is methyl;
9) 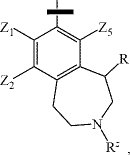
wherein Z
₁, Z₂, Z₅ are H, R^{z}, R are methyl;
R², R³ each are independently selected from:
H, C₁—C₆ alkyl, C₁—C₆ fluorine-containing alkyl, C₃—C₆ cycloalkyl, or C₃—C₆ fluorine-containing cycloalkyl;
preferably, R², R³ each are independently selected from H, methyl, trifluoromethyl, ethyl, isopropyl, or cyclopropyl;
R⁴, R⁵ each are independently selected from:
H, F, Cl, Br, C₁—C₆ fluorine-containing alkyl (e.g., trifluoromethyl), cyano, nitro, C₁—C₆ alkyl (such as, methyl, ethyl or isopropyl), C₁—C₆ alkoxy (such as, methoxy or ethoxy), or C₃—C₆ cycloalkyl (e.g., cyclopropyl),
or, R⁴, R⁵ and carbon atoms linked thereto together form a N—, O— or S-containing 5-membered ring;
preferably, R⁴ is H, R⁵ is selected from H, F, Cl, Br, trifluoromethyl, cyano, nitro, methyl, ethyl, isopropyl, methoxy, ethoxy or cyclopropyl,
or, R⁴, R⁵ and carbon atoms linked thereto together form 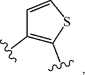 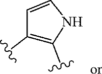 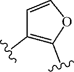
.

2. The compound according to claim 1, having the following formula: 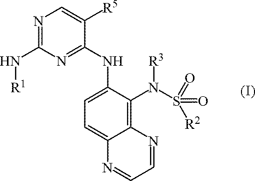
or a stereoisomer, a prodrug, a pharmaceutically acceptable salt or a pharmaceutically acceptable solvate thereof, wherein:
R¹ is selected from:
1) H, C₁—C₆ alkyl, C₁—C₆ fluorine-containing alkyl, C₃—C₈ cycloalkyl, C₁—C₆ heteroatom-containing alkyl, C₃—C₈ heteroatom-containing cycloalkyl;
2) 2-N,N-dimethylaminoethyl, 3-N,N-dimethylaminopropyl, 2-N,N-diethylaminoethyl, 3-N,N-diethylaminopropyl, 2-(N-methylpiperazinyl)ethyl, 2-(N-acetylpiperazinyl)ethyl, 2-morpholinylethyl, 2-thiomorpholinylethyl, 2-piperidylethyl, 2-hydroxyethyl, 3-hydroxypropyl, 2-methoxyethyl, N-methyl-4-piperidyl, N-ethyl-4-piperidyl, N-propyl-4-piperidyl, N-isopropyl-4-piperidyl, N-hydroxyethyl-4-piperidyl, N-cyanomethyl-4-piperidyl, N-(2-cyanoethyl)-4-piperidyl, N-acetyl-4-piperidyl, N-propionyl-4-piperidyl, N-isopropionyl-4-piperidyl, N-cyclopropylformyl-4-piperidyl, N-methylsulfonyl-4-piperidyl, N-ethylsulfonyl-4-piperidyl, N-propylsulfonyl-4-piperidyl, N-isopropylsulfonyl-4-piperidyl, N-cyclopropylsulfonyl-4-piperidyl, 3-N,N-dimethylaminocyclopentyl, 3-N,N-diethylam inocyclopentyl, 3-N,N-dipropylaminocyclopentyl, 3-N,N-isopropylam inocyclopentyl, 4-aminocyclohexyl, 4-N,N-dimethylaminocyclohexyl, 4-N,N-diethylam inocyclohexyl, 4-N,N-dipropylaminocyclohexyl, 4-N,N-diisopropylaminocyclohexyl, 4-acetylaminocyclohexyl, 4-propionylam inocyclohexyl, 4-isopropylformylaminocyclohexyl, 4-methylsulfonylaminocyclohexyl, 4-ethylsulfonylaminocyclohexyl, 4-propylsulfonylaminocyclohexyl, 4-isopropylsulfonylaminocyclohexyl, 4-cyclopropylsulfonylaminocyclohexyl, 4-hydroxycyclohexyl, 4-methoxycyclohexyl, 4-ethoxycyclohexyl, 4-isopropoxycyclohexyl;
3) 1-methyl-3-pyrrolyl, 1-ethyl-3-pyrrolyl, 1-isopropyl-3-pyrrolyl, 1-cyclopropyl-3-pyrrolyl, 1-methyl-3-pyrazolyl, 1-ethyl-3-pyrazolyl, 1-isopropyl-3-pyrazolyl, 1-cyclopropyl-3-pyrazolyl, 1-hydroxyethyl-3-pyrazolyl, 1,5-dimethyl-3-pyrazolyl, 1,5-diethyl-3-pyrazolyl, 1,5-diisopropyl-3-pyrazolyl, 1-methyl-4-pyrazolyl, 1-ethyl-4-pyrazolyl, 1-isopropyl-4-pyrazolyl, 1,3-dimethyl-4-pyrazolyl, 1 -methyl-3-ethyl-4-pyrazolyl, 1-methyl-3-isopropyl-4-pyrazolyl, 1-ethyl-3-methyl-4-pyrazolyl, 1,3-diethyl-4-pyrazolyl, 1-ethyl-3-isopropyl-4-pyrazolyl, 1-isopropyl-3-methyl-4-pyrazolyl, 1-isopropyl-3-ethyl-4-pyrazolyl, 1,3-diisopropyl-4-pyrazolyl, 1-cyclopropyl-4-pyrazolyl, 1-hydroxyethyl-4-pyrazolyl, 1-(N-methyl-4-piperidyl)-4-pyrazolyl, 1-(N-t-butoxycarbonyl-4-piperidyl)-4-pyrazolyl, 1,3-dimethyl-5-pyrazolyl, 1-methyl-4-imidazolyl, 3-methyl-5-isoxazolyl;
4) 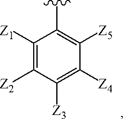
wherein Z
₁, Z₂, Z₃, Z₄, Z₅ each are independently selected from the following groups, and Z₁, Z₂, Z₃, Z₄, Z₅ are not hydrogen at the same time:
(1) H, F, Cl, Br, I, nitro, cyano,
(2) C₁—C₆ alkyl, C₁—C₆ alkoxy, C₁—C₆ oxygen-containing alkyl, C₁—C₆ fluorine-containing alkyl, C₁—C₆ fluorine-containing alkoxy, C₃—C₆ cycloalkyl, C₂—C₆ alkenyl,
(3) 2-N,N-dimethylaminoethoxy, 3-N,N-dimethylaminopropoxy, 2-(N-methylpiperazinyl)ethoxy, 2-(N-acetylpiperazinyl)ethoxy, 2-morpholinylethoxy, 2-thiomorpholinylethoxy, 2-piperidylethoxy, 3-N,N-dimethylaminopropoxy, 3-N,N-diethylam inopropoxy, 3-N,N-diisopropylaminopropoxy, 3-(N-methylpiperazinyl)propoxy, 3-(N-acetylpiperazinyl)propoxy, 3-morpholinylpropoxy, 3-thiomorpholinylpropoxy, 3-piperidylpropoxy, 2-pyridylmethoxy, 3-pyridylmethoxy, 4-pyridylmethoxy, phenylmethoxy, monohalo-substituted phenylmethoxy, gem-dihalo-substituted phenylmethoxy, hetero-dihalo-substituted phenylmethoxy,
(4) piperazinyl, N-methylpiperazinyl, N-ethylpiperazinyl, N-n-propylpiperazinyl, N-isopropylpiperazinyl, 4-(piperidyl-4-)piperazinyl, 4-(N-methylpiperidyl-4-)piperazinyl, 4-(N-ethylpiperidyl-4-)piperazinyl, 4-(N-n-propylpiperidyl-4-)piperazinyl, 4-(N-isopropylpiperidyl-4-)piperazinyl, 4-(azetidin-3-yl)piperazinyl, 4-(N-methyl-azetidin-3-yl)piperazinyl, 4-(N-ethyl-azetidin-3-yl)piperazinyl, 4-(N-n-propyl-azetidin-3-yl)piperazinyl, 4-(N-isopropyl-azetidin-3-yl)piperazinyl, N-cyclopropylpiperazinyl, N-(2-hydroxyethyl)piperazinyl, N-(2-N,N-dimethylaminoethyl)piperazinyl, N-(2-N,N-diethylaminoethyl)piperazinyl, N-(2-cyanoethyl)piperazinyl, N-cyanomethylpiperazinyl, N-(3-hydroxypropyl)piperazinyl, N-(3-N,N-dimethylaminopropyl)piperazinyl, N-(3-N,N-diethylaminopropyl)piperazinyl, N-(N-methyl-4-piperidyl)piperazinyl, N-(N-ethyl-4-piperidyl)piperazinyl, N-t-butoxycarbonylpiperazinyl, N-acetylpiperazinyl, N-propionylpiperazinyl, N-isobutyrylpiperazinyl, N-cyclopropylformylpiperazinyl, N-methylsulfonylpiperazinyl, N-ethylsulfonylpiperazinyl, N-n-propylsulfonylpiperazinyl, N-isopropylsulfonylpiperazinyl, N-cyclopropylsulfonylpiperazinyl, 2-oxo-piperazin-4-yl,
(5) morpholinyl, 2,6-dimethylmorpholinyl, thiomorpholinyl,
(6) tetrahydropyrrolyl, 3-(N,N-dimethylamino)tetrahydropyrrolyl, 3-(N,N-diethylam ino)tetrahydropyrrolyl,
(7) imidazolyl, 4-methyl-1-imidazolyl,
(8) piperidyl, 4-N,N-dimethylaminopiperidyl, 4-N,N-diethylaminopiperidyl, 4-N,N-diisopropylaminopiperidyl, 4-hydroxypiperidyl, 4-cyanopiperidyl, 4-methoxypiperidyl, 4-ethoxypiperidyl, 4-(piperazinyl-1-)piperidyl, 4-(N-methylpiperazinyl-1-)piperidyl, 4-(N-ethylpiperazinyl-1-)piperidyl, 4-(N-isopropylpiperazinyl-1-)piperidyl, 4-(N-acetylpiperazinyl-1-)piperidyl, 4-(N-t-butoxycarbonylpiperazinyl-1-)piperidyl, 4-(N-methylsulfonylpiperazinyl-1-)piperidyl, 4-(N-(2-hydroxyethyl)piperazinyl-1-)piperidyl, 4-(N-(2-cyanoethyl)piperazinyl-1-)piperidyl, 4-(N-(3-hydroxypropyl)piperazinyl-1-)piperidyl, 4-(N-(2-N,N-dimethylaminoethyl)piperazinyl-1 -)piperidyl, 4-(N-(2-N,N-diethylethyl)piperazinyl-1-)piperidyl, 4-(N-(3-N,N-dimethylpropyl)piperazinyl-1-)piperidyl, 4-(N-(3-N,N-diethylpropyl)piperazinyl-1-)piperidyl, 4-(tetrahydropyrrolyl-1-)piperidyl, 4-(3-N, N-dimethylaminotetrahydropyrrolyl-1-)piperidyl, 4-(3-N, N-diethylam inotetrahydropyrrolyl-1-)piperidyl,
(9) piperidin-4-yl, N-methyl-4-piperidyl, N-ethyl-4-piperidyl, N-isopropyl-4-piperidyl, N-acetyl-4-piperidyl, N-t-butoxycarbonyl-4-piperidyl, N-methylsulfonyl-4-piperidyl, N-(2-hydroxyethyl)-4-piperidyl, N-(2-N,N-dimethylaminoethyl)-4-piperidyl, N-(2-N,N-diethylam inoethyl)-4-piperidyl, N-(2-cyanoethyl)-4-piperidyl, N-(3-hydroxypropyl)-4-piperidyl, N-(3-N,N-dimethylaminopropyl)-4-piperidyl, N-(3-N,N-diethylam inopropyl)-4-piperidyl, N-(3-cyanopropyl)-4-piperidyl, N-cyanomethylene-4-piperidyl,
(10) amino, N,N-dimethylamino, N,N-diethylamino, N,N-di-n-propylamino, N,N-diisopropylamino, 2-hydroxyethylamino, 2-N,N-dimethylaminoethylamino, 2-morpholinylethylamino, 2-(N-methylpiperazinyl)ethylamino, 3-N,N-dimethylaminopropylamino, 3-N,N-diethylaminopropylamino, 3-N,N-diisopropylaminopropylamino, 3-morpholinylpropylamino, 3-(N-methylpiperazinyl)propylamino, N-methylpiperidyl-4-amino, N-ethylpiperidyl-4-amino, N-n-propylpiperidyl-4-amino, N-isopropylpiperidyl-4-amino, acetamido, propionamido, 2-(N,N-dimethylamino)acetamido, 2-hydroxyacetamido, 2-methoxyacetamido, methylsulfamido, ethylsulfamido, n-propylsulfamido, isopropylsulfamido, cyclopropylsulfamido, N-methyl-N-(N-methyl-3-tetrahydropyrrolyl)amino, N-(N-methyl-3-tetrahydropyrrolyl)amino, N-methyl-N-(2-N,N-dimethylamino)ethylamino,
(11) aminomethylene, N,N-dimethylaminomethylene, N,N-diethylaminomethylene, 4-methyl-1-piperazinylmethylene, 4-ethyl-1-piperazinylmethylene, 4-propyl-1-piperazinylmethylene, 4-isopropyl-1-piperazinylmethylene, 4-(2-hydroxyethyl)-1-piperazinylmethylene, 4-(2-N,N-dimethylaminoethyl)-1-piperazinylmethylene, piperidyl-1-methylene, 4-(N,N-dimethylamino)-1-piperidylmethylene, 4-hydroxypiperidyl-1-methylene, tetrahydropyrrolyl-1-methylene, 3-(N,N-dimethylamino)-1-tetrahydropyrrolylmethylene, benzyl, cyclopropylmethylene, cyclobutylmethylene, cyclopentylmethylene, cyclohexylmethylene,
(12) aminoformyl, methylaminoformyl, N,N-dimethylaminoformyl, ethylaminoformyl, cyclopropylaminoformyl, cyclobutylaminoformyl, cyclopentylaminoformyl, cyclohexylaminoformyl, aminoacetyl, methylaminoacetyl, 2-(N,N-dimethylamino)acetyl, ethylaminoacetyl, cyclopropylaminoacetyl, cyclobutylaminoacetyl, cyclopentylaminoacetyl, cyclohexylaminoacetyl, tetrahydropyrrolyl-1-formyl, 3-(N,N-dimethylamino)tetrahydropyrrolyl-1-formyl, 3-(N,N-diethylamino)tetrahydropyrrolyl-1-formyl, morpholinyl-4-formyl, thiomorpholinyl-4-formyl, 2,6-dimethylmorpholinyl-4-formyl, piperidyl-1-formyl, 4-hydroxypiperidyl-1-formyl, 4-(N,N-dimethylamino)piperidyl-1-formyl, 4-(N,N-diethylamino)piperidyl-1-formyl, 4-(N-methyl-1-piperazinyl)piperidyl-1-formyl, 4-(N-ethyl-1-piperazinyl)piperidyl-1-formyl, N-methylpiperazinyl-1-formyl, N-ethylpiperazinyl-1-formyl, N-isopropylpiperazinyl-1-formyl, N-(2-hydroxyethyl)piperazinyl-1-formyl, N-(2-N,N-dimethylaminoethyl)piperazinyl-1-formyl, N-(2-N,N-diethylaminoethyl)piperazinyl-1-formyl, 4-(N-methyl-4-piperidyl)piperazinyl-1-formyl, 4-(N-ethyl-4-piperidyl)piperazinyl-1-formyl,
(13) hydroxyformyl, methoxyformyl, ethoxyformyl, propoxyformyl, isopropoxyformyl, n-butoxyformyl, iso-butoxyformyl, t-butoxyformyl,
(14) hydroxysulfonyl, aminosulfonyl, methylaminosulfonyl, ethylaminosulfonyl, propylaminosulfonyl, isopropylaminosulfonyl, cyclopropylaminosulfonyl, cyclobutylaminosulfonyl, cyclopentylaminosulfonyl, cyclohexylaminosulfonyl, tetrahydropyrrolyl-1-sulfonyl, 3-(N,N-dimethylamino)tetrahydropyrrolyl-1-sulfonyl, 3-(N,N-diethylamino)tetrahydropyrrolyl-1-sulfonyl, morpholinyl-4-sulfonyl, thiomorpholinyl-4-sulfonyl, 2,6-dimethylmorpholinyl-4-sulfonyl, piperidyl-1-sulfonyl, 4-hydroxypiperidyl-1-sulfonyl, 4-(N,N-dimethylamino)piperidyl-1-sulfonyl, 4-(N,N-diethylamino)piperidyl-1-sulfonyl, 4-(N-methyl-1-piperazinyl)piperidyl-1-sulfonyl, 4-(N-ethyl-1-piperazinyl)piperidyl-1-sulfonyl, N-methylpiperazinyl-1-sulfonyl, N-ethylpiperazinyl-1-sulfonyl, N-isopropylpiperazinyl-1-sulfonyl, N-(2-hydroxyethyl)piperazinyl-1-sulfonyl, N-(2-N,N-dimethylaminoethyl)piperazinyl-1-sulfonyl, N-(2-N,N-diethylaminoethyl)piperazinyl-1-sulfonyl, 4-(N-methyl-4-piperidyl)piperazinyl-1-sulfonyl, 4-(N-ethyl-4-piperidyl)piperazinyl-1-sulfonyl,
(15) aminoformamido, methylaminoformamido, ethylaminoformamido, propylaminoformamido, isopropylaminoformamido, cyclopropylaminoformamido, cyclobutylaminoformamido, cyclopentylaminoformamido, piperidyl-1-formamido, 4-hydroxypiperidyl-1-formamido, 4-N,N-dimethylpiperidyl-1-formamido, 4-N,N-diethylpiperidyl-1-formamido, tetrahydropyrrolyl-1-formam ido, 3-N,N-dimethyltetrahydropyrrolyl-1-formamido, 3-N,N-diethyltetrahydropyrrolyl-1-formamido, N-methylpiperazinyl-1-formamido, N-ethylpiperazinyl-1-formamido, N-acetylpiperazinyl-1-formamido, N-t-butoxycarbonylpiperazinyl-1-formamido, N-(2-hydroxyethyl)piperazinyl-1-formamido, N-(2-cyanoethyl)piperazinyl-1-formamido, N-(2-N,N-dimethylaminoethyl)piperazinyl-1-formamido, N-(2-N,N-diethylam inoethyl)piperazinyl-1-formamido, N-(3-hydroxypropyl)piperazinyl-1 -formamido, N-(3-N,N-dimethylpropyl)piperazinyl-1-formamido, N-(3-N,N-diethylpropyl)piperazinyl-1-formamido, morpholinyl-1-formamido, 3,5-dimethylmorpholinyl-1-formamido, 4-(tetrahydropyrrolyl)piperidyl-1-formamido, 4-(N-methyl-1-piperazinyl)piperidyl-1-formamido, 4-(N-ethyl-1-piperazinyl)piperidyl-1-formamido, 4-(N-acetyl-1-piperazinyl)piperidyl-1-formamido, N-(N-methyl-4-piperidyl)piperazinyl-1-formamido,
(16) 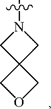 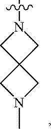 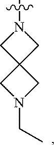 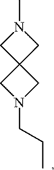 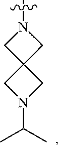 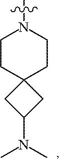 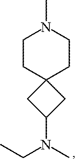 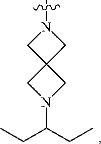 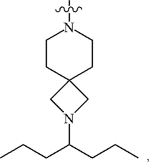 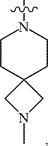 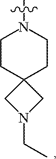 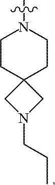 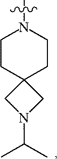
(17) Z₃ and Z₄ may form an oxygen-containing substituted or unsubstituted 5- to 7-membered ring; the substituent may be selected from the same substituents as described above for Z₁,
(18) Z₃ and Z₄ may form a nitrogen-containing substituted or unsubstituted 5- to 7-membered ring; the substituent may be selected from the same substituents as described above for Z₁;
5) 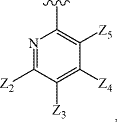
wherein Z
₂, Z₃, Z₄, Z₅ are the same as defined in 4), and Z₂, Z₃, Z₄, Z₅ are not hydrogen at the same time;
6) 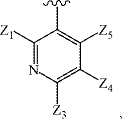
wherein Z
₁, Z₃, Z₄, Z₅ are the same as defined in 4), and Z₁, Z₃, Z₄, Z₅ are not hydrogen at the same time;
7) 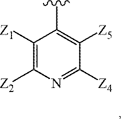
wherein Z
₁, Z₂, Z₄, Z₅ are the same as defined in 4), and Z₁, Z₂, Z₄, Z₅ may be hydrogen at the same time;
8) 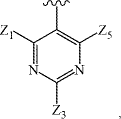
wherein Z
₁, Z₃, Z₅ are the same as defined in 4), and Z₁, Z₃, Z₅ are not hydrogen at the same time;
preferably, R¹ is selected from:
1) H, C₁—C₆ alkyl, C₁—C₆ fluorine-containing alkyl, C₃—C₈ cycloalkyl, C₁—C₆ heteroatom-containing alkyl, C₃—C₈ heteroatom-containing cycloalkyl;
2) 2-N,N-dimethylaminoethyl, 3-N,N-dimethylaminopropyl, 2-N,N-diethylaminoethyl, 3-N,N-diethylaminopropyl, 2-(N-methylpiperazinyl)ethyl, 2-(N-acetylpiperazinyl)ethyl, 2-morpholinylethyl, 2-thiomorpholinylethyl, 2-piperidylethyl, 2-hydroxyethyl, 3-hydroxypropyl, 2-methoxyethyl, N-methyl-4-piperidyl, N-ethyl-4-piperidyl, N-propyl-4-piperidyl, N-isopropyl-4-piperidyl, N-hydroxyethyl-4-piperidyl, N-cyanomethyl-4-piperidyl, N-(2-cyanoethyl)-4-piperidyl, 3-N,N-dimethylaminocyclopentyl, 3-N,N-diethylaminocyclopentyl, 3-N,N-dipropylaminocyclopentyl, 3-N,N-isopropylaminocyclopentyl, 4-aminocyclohexyl, 4-N,N-dimethylaminocyclohexyl, 4-N,N-diethylaminocyclohexyl, 4-N,N-dipropylaminocyclohexyl, 4-N,N-diisopropylaminocyclohexyl, 4-hydroxycyclohexyl, 4-methoxycyclohexyl, 4-ethoxycyclohexyl, 4-isopropoxycyclohexyl;
3) 1-methyl-3-pyrrolyl, 1-ethyl-3-pyrrolyl, 1-isopropyl-3-pyrrolyl, 1-cyclopropyl-3-pyrrolyl, 1-methyl-3-pyrazolyl, 1-ethyl-3-pyrazolyl, 1-isopropyl-3-pyrazolyl, 1-cyclopropyl-3-pyrazolyl, 1-hydroxyethyl-3-pyrazolyl, 1,5-dimethyl-3-pyrazolyl, 1,5-diethyl-3-pyrazolyl, 1,5-diisopropyl-3-pyrazolyl, 1-methyl-4-pyrazolyl, 1-ethyl-4-pyrazolyl, 1-isopropyl-4-pyrazolyl, 1-cyclopropyl-4-pyrazolyl, 1-hydroxyethyl-4-pyrazolyl, 1,3-dimethyl-4-pyrazolyl, 1-methyl-3-ethyl-4-pyrazolyl, 1-methyl-3-isopropyl-4-pyrazolyl, 1-ethyl-3-methyl-4-pyrazolyl, 1,3-diethyl-4-pyrazolyl, 1-ethyl-3-isopropyl-4-pyrazolyl, 1-isopropyl-3-methyl-4-pyrazolyl, 1-isopropyl-3-ethyl-4-pyrazolyl, 1,3-diisopropyl-4-pyrazolyl, 1,3-dimethyl-5-pyrazolyl, 1-methyl-4-imidazolyl, 3-methyl-5-isoxazolyl;
4) 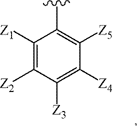
wherein Z
₁, Z₂, Z₃, Z₄, Z₅ each are independently selected from the following groups, and Z₁, Z₂, Z₃, Z₄, Z₅ are not hydrogen at the same time:
(1) H, F, Cl, Br, I, nitro, cyano,
(2) C₁—C₆ alkyl, C₁—C₆ alkoxy, C₁—C₆ oxygen-containing alkyl, C₁—C₆ fluorine-containing alkyl, C₁—C₆ fluorine-containing alkoxy, C₃—C₆ cycloalkyl, C₂—C₆ alkenyl,
(3) piperazinyl, N-methylpiperazinyl, N-ethylpiperazinyl, N-n-propylpiperazinyl, N-isopropylpiperazinyl, 4-(piperidyl-4-)piperazinyl, 4-(N-methylpiperidyl-4-)piperazinyl, 4-(N-ethylpiperidyl-4-)piperazinyl, 4-(N-n-propylpiperidyl-4-)piperazinyl, 4-(N-isopropylpiperidyl-4-)piperazinyl, 4-(azetidin-3-yl)piperazinyl, 4-(N-methyl-azetidin-3-yl)piperazinyl, 4-(N-ethyl-azetidin-3-yl)piperazinyl, 4-(N-n-propyl-azetidin-3-yl)piperazinyl, 4-(N-isopropyl-azetidin-3-yl)piperazinyl, N-cyclopropylpiperazinyl, N-(2-hydroxyethyl)piperazinyl, N-(2-N,N-dimethylaminoethyl)piperazinyl, N-(2-N,N-diethylaminoethyl)piperazinyl, N-(2-cyanoethyl)piperazinyl, N-cyanomethylpiperazinyl, N-(3-hydroxypropyl)piperazinyl, N-(3-N,N-dimethylaminopropyl)piperazinyl, N-(3-N,N-diethylaminopropyl)piperazinyl,
(4) morpholinyl, 2,6-dimethylmorpholinyl, thiomorpholinyl,
(5) tetrahydropyrrolyl, 3-(N,N-dimethylamino)tetrahydropyrrolyl, 3-(N,N-diethylam ino)tetrahydropyrrolyl,
(6) imidazolyl, 4-methyl-1-imidazolyl,
(7) piperidyl, 4-N,N-dimethylaminopiperidyl, 4-N,N-diethylaminopiperidyl, 4-N,N-diisopropylaminopiperidyl, 4-hydroxypiperidyl, 4-cyanopiperidyl, 4-methoxypiperidyl, 4-ethoxypiperidyl, 4-(piperazinyl-1-)piperidyl, 4-(N-methylpiperazinyl-1-)piperidyl, 4-(N-ethylpiperazinyl-1-)piperidyl, 4-(N-isopropylpiperazinyl-1-)piperidyl, 4-(N-(2-hydroxyethyl)piperazinyl-1-)piperidyl, 4-(N-(2-cyanoethyl)piperazinyl-1-)piperidyl, 4-(N-(3-hydroxypropyl)piperazinyl-1-)piperidyl, 4-(N-(2-N,N-dimethylaminoethyl)piperazinyl-1-)piperidyl, 4-(N-(2-N,N-diethylethyl)piperazinyl-1 -)piperidyl, 4-(N-(3-N,N-dimethylpropyl)piperazinyl-1-)piperidyl, 4-(N-(3-N,N-diethylpropyl)piperazinyl-1-)piperidyl, 4-(tetrahydropyrrolyl-1-)piperidyl, 4-(3-N, N-dimethylaminotetrahydropyrrolyl-1-)piperidyl, 4-(3-N, N-diethylam inotetrahydropyrrolyl-1-)piperidyl,
(8) piperidin-4-yl, N-methyl-4-piperidyl, N-ethyl-4-piperidyl, N-isopropyl-4-piperidyl, N-(2-hydroxyethyl)-4-piperidyl, N-(2-N,N-dimethylam inoethyl)-4-piperidyl, N-(2-N,N-diethylam inoethyl)-4-piperidyl, N-(2-cyanoethyl)-4-piperidyl, N-(3-hydroxypropyl)-4-piperidyl, N-(3-N,N-dimethylam inopropyl)-4-piperidyl, N-(3-N,N-diethylam inopropyl)-4-piperidyl, N-(3-cyanopropyl)-4-piperidyl, N-cyanomethylene-4-piperidyl,
(9) amino, N,N-dimethylamino, N,N-diethylamino, N,N-di-n-propylamino, N,N-diisopropylamino, 2-hydroxyethylamino, 2-N,N-dimethylaminoethylamino, 3-N,N-dimethylam inopropylamino, 3-N,N-diethylam inopropylamino, 3-N,N-diisopropylaminopropylamino, N-methylpiperidyl-4-amino, N-ethylpiperidyl-4-amino, N-n-propylpiperidyl-4-amino, N-isopropylpiperidyl-4-amino, acetamido, propionamido, 2-(N,N-dimethylamino)acetamido, 2-hydroxyacetamido, 2-methoxyacetamido, methylsulfamido, ethylsulfamido, n-propylsulfamido, isopropylsulfamido, cyclopropylsulfamido, N-methyl-N-(N-methyl-3-tetrahydropyrrolyl)amino, N-(N-methyl-3-tetrahydropyrrolyl)amino, N-methyl-N-(2-N,N-dimethylamino)ethylamino,
(10) 4-methyl-1-piperazinylmethylene, 4-ethyl-1-piperazinylmethylene, 4-propyl-1-piperazinylmethylene, 4-isopropyl-1-piperazinylmethylene, 4-(2-hydroxyethyl)-1-piperazinylmethylene, 4-(2-N,N-dimethylam inoethyl)-1-piperazinylmethylene, piperidyl-1-methylene, 4-(N,N-dimethylamino)-1-piperidylmethylene, 4-hydroxypiperidyl-1-methylene, tetrahydropyrrolyl-1-methylene, 3-(N,N-dimethylamino)-1-tetrahydropyrrolylmethylene, benzyl, cyclopropylmethylene, cyclobutylmethylene, cyclopentylmethylene, cyclohexylmethylene,
(11) aminoformyl, methylaminoformyl, N,N-dimethylaminoformyl, ethylaminoformyl, aminoacetyl, methylaminoacetyl, 2-(N,N-dimethylamino)acetyl, ethylaminoacetyl, tetrahydropyrrolyl-1-formyl, 3-(N,N-dimethylam ino)tetrahydropyrrolyl-1-formyl, 3-(N,N-diethylamino)tetrahydropyrrolyl-1-formyl, morpholinyl-4-formyl, thiomorpholinyl-4-formyl, 2,6-dimethylmorpholinyl-4-formyl, piperidyl-1-formyl, 4-hydroxypiperidyl-1-formyl, 4-(N,N-dimethylamino)piperidyl-1-formyl, 4-(N,N-diethylamino)piperidyl-1-formyl, 4-(N-methyl-1-piperazinyl)piperidyl-1-formyl, 4-(N-ethyl-1-piperazinyl)piperidyl-1-formyl, N-methylpiperazinyl-1-formyl, N-ethylpiperazinyl-1-formyl, N-isopropylpiperazinyl-1-formyl, N-(2-hydroxyethyl)piperazinyl-1-formyl, N-(2-N,N-dimethylam inoethyl)piperazinyl-1 -formyl, N-(2-N,N-diethylam inoethyl)piperazinyl-1 -formyl, 4-(N-methyl-4-piperidyl)piperazinyl-1-formyl, 4-(N-ethyl-4-piperidyl)piperazinyl-1-formyl,
(12) hydroxysulfonyl, aminosulfonyl, methylaminosulfonyl, ethylaminosulfonyl, propylaminosulfonyl, isopropylaminosulfonyl, cyclopropylaminosulfonyl, cyclobutylaminosulfonyl, cyclopentylaminosulfonyl, cyclohexylaminosulfonyl, tetrahydropyrrolyl-1-sulfonyl, 3-(N,N-dimethylamino)tetrahydropyrrolyl-1-sulfonyl, 3-(N,N-diethylamino)tetrahydropyrrolyl-1-sulfonyl, morpholinyl-4-sulfonyl, thiomorpholinyl-4-sulfonyl, 2,6-dimethylmorpholinyl-4-sulfonyl, piperidyl-1-sulfonyl, 4-hydroxypiperidyl-1-sulfonyl, 4-(N,N-dimethylamino)piperidyl-1-sulfonyl, 4-(N,N-diethylamino)piperidyl-1-sulfonyl, N-methylpiperazinyl-1-sulfonyl, N-ethylpiperazinyl-1-sulfonyl, N-isopropylpiperazinyl-1-sulfonyl, N-(2-hydroxyethyl)piperazinyl-1-sulfonyl, N-(2-N,N-dimethylam inoethyl)piperazinyl-1-sulfonyl, N-(2-N,N-diethylam inoethyl)piperazinyl-1-sulfonyl,
(13) aminoformamido, methylaminoformamido, ethylaminoformamido, propylaminoformamido, isopropylaminoformamido,
(14) 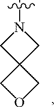 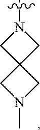 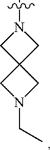 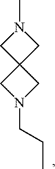 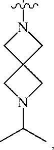 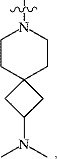 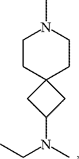 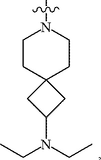 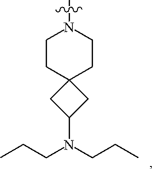 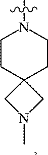 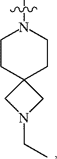 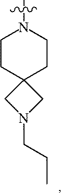 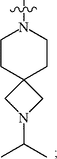
5) 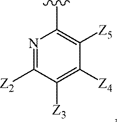
wherein Z
₂, Z₃, Z₄, Z₅ are the same as defined in 4), and Z₂, Z₃, Z₄, Z₅ are not hydrogen at the same time;
6) 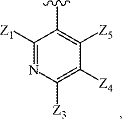
wherein Z
₁, Z₃, Z₄, Z₅ are the same as defined in 4), and Z₁, Z₃, Z₄, Z₅ are not hydrogen at the same time;
7) 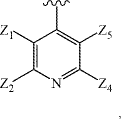
wherein Z
₁, Z₂, Z₄, Z₅ are the same as defined in 4), and Z₁, Z₂, Z₄, Z₅ may be hydrogen at the same time;
8) 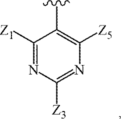
wherein Z
₁, Z₃, Z₅ are the same as defined in 4), and Z₁, Z₃, Z₅ are not hydrogen at the same time;
9) 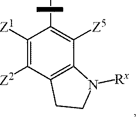
wherein Z
₁, Z₂, Z₅ are the same as defined in 4), and Z₁, Z₂, Z₅ may be hydrogen at the same time, R^{x} is selected from the same substituents as described above for Z¹;
10) 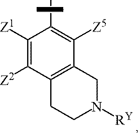
wherein Z
₁, Z₂, Z₅ are the same as defined in 4), and Z₁, Z₂, Z₅ may be hydrogen at the same time, R^{y} is selected from the same substituents as described above for Z¹;
11) 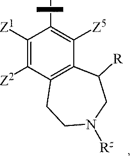
wherein Z
₁, Z₂, Z₅ are the same as defined in 4), and Z₁, Z₂, Z₅ may be hydrogen at the same time, R^{z}, R each are independently selected from the same substituents as described above for Z¹;
more preferably, R¹ is selected from:
1) H, C₁—C₆ alkyl, C₃—C₈ cycloalkyl;
2) 2-N,N-dimethylaminoethyl, 3-N,N-dimethylaminopropyl, 2-N,N-diethylaminoethyl, 3-N,N-diethylaminopropyl, 2-hydroxyethyl, 3-hydroxypropyl, N-methyl-4-piperidyl, N-ethyl-4-piperidyl, N-propyl-4-piperidyl, N-isopropyl-4-piperidyl, 4-aminocyclohexyl, 4-N,N-dimethylaminocyclohexyl, 4-N,N-diethylaminocyclohexyl, 4-N,N-dipropylaminocyclohexyl, 4-N,N-diisopropylaminocyclohexyl;
3) 1-methyl-3-pyrazolyl, 1-ethyl-3-pyrazolyl, 1-isopropyl-3-pyrazolyl, 1-cyclopropyl-3-pyrazolyl, 1,5-dimethyl-3-pyrazolyl, 1,5-diethyl-3-pyrazolyl, 1,5-diisopropyl-3-pyrazolyl, 1-methyl-4-pyrazolyl, 1-ethyl-4-pyrazolyl, 1-isopropyl-4-pyrazolyl, 1,3-dimethyl-4-pyrazolyl, 1-methyl-3-ethyl-4-pyrazolyl, 1-methyl-3-isopropyl-4-pyrazolyl, 1-ethyl-3-methyl-4-pyrazolyl, 1,3-diethyl-4-pyrazolyl, 1-ethyl-3-isopropyl-4-pyrazolyl, 1-isopropyl-3-methyl-4-pyrazolyl, 1-isopropyl-3-ethyl-4-pyrazolyl, 1,3-diisopropyl-4-pyrazolyl, 1-cyclopropyl-4-pyrazolyl, 1,3-dimethyl-5-pyrazolyl, 1-methyl-4-imidazolyl;
4) 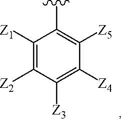
wherein Z
₁, Z₂, Z₃, Z₄, Z₅ each are independently selected from the following groups, and Z₁, Z₂, Z₃, Z₄, Z₅ are not hydrogen at the same time:
(1) H, F, Cl, Br, I, nitro, cyano,
(2) C₁—C₆ alkyl, C₁—C₆ alkoxy, C₁—C₆ fluorine-containing alkyl, C₁—C₆ fluorine-containing alkoxy, C₂—C₆ alkenyl, C₃—C₆ cycloalkyl,
(3) piperazinyl, N-methylpiperazinyl, N-ethylpiperazinyl, N-n-propylpiperazinyl, N-isopropylpiperazinyl, 4-(piperidyl-4-)piperazinyl, 4-(N-methylpiperidyl-4-)piperazinyl, 4-(N-ethylpiperidyl-4-)piperazinyl, 4-(N-n-propylpiperidyl-4-)piperazinyl, 4-(N-isopropylpiperidyl-4-)piperazinyl, 4-(azetidin-3-yl)piperazinyl, 4-(N-methyl-azetidin-3-yl)piperazinyl, 4-(N-ethyl-azetidin-3-yl)piperazinyl, 4-(N-n-propyl-azetidin-3-yl)piperazinyl, 4-(N-isopropyl-azetidin-3-yl)piperazinyl,
(4) morpholinyl, 2,6-dimethylmorpholinyl, thiomorpholinyl,
(5) 3-(N,N-dimethylamino)tetrahydropyrrolyl, 3-(N,N-diethylamino)tetrahydropyrrolyl,
(6) 4-N,N-dimethylaminopiperidyl, 4-N,N-diethylaminopiperidyl, 4-N,N-diisopropylaminopiperidyl, 4-hydroxypiperidyl, 4-cyanopiperidyl, 4-(piperazinyl-1-)piperidyl, 4-(N-methylpiperazinyl-1-)piperidyl, 4-(N-ethylpiperazinyl-1-)piperidyl, 4-(N-isopropylpiperazinyl-1-)piperidyl, 4-(tetrahydropyrrolyl-1-)piperidyl,
(7) piperidin-4-yl, N-methyl-4-piperidyl, N-ethyl-4-piperidyl, N-isopropyl-4-piperidyl,
(8) aminoformamido, methylaminoformamido, ethylaminoformamido, propylaminoformamido, isopropylaminoformamido, 2-(N,N-dimethylamino)acetamido, cyclopropylsulfamido, 2-hydroxyethylamino, 2-N,N-dimethylaminoethylamino, 3-N,N-dimethylaminopropylamino, 3-N,N-diethylaminopropylamino, 3-N,N-diisopropylaminopropylamino, N-methyl-N-(2-N,N-dimethylamino)ethylamino,
(9) 4-methyl-1-piperazinylmethylene, 4-ethyl-1-piperazinylmethylene, 4-propyl-1-piperazinylmethylene, 4-isopropyl-1-piperazinylmethylene, benzyl, cyclopropylmethylene, cyclobutylmethylene, cyclopentylmethylene, cyclohexylmethylene,
(10) 4-(N-methyl-1-piperazinyl)piperidyl-1-formyl, 4-(N-ethyl-1-piperazinyl)piperidyl-1-formyl, N-methylpiperazinyl-1-formyl, N-ethylpiperazinyl-1-formyl, N-isopropylpiperazinyl-1-formyl,
(11) cyclopropylaminosulfonyl, cyclobutylaminosulfonyl, cyclopentylaminosulfonyl, cyclohexylaminosulfonyl, N-methylpiperazinyl-1-sulfonyl, N-ethylpiperazinyl-1-sulfonyl, N-isopropylpiperazinyl-1-sulfonyl,
(12) 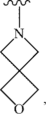 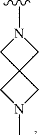 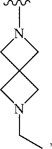 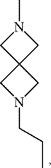 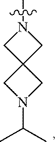 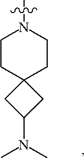 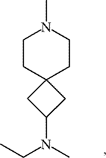 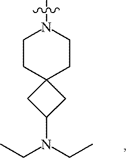 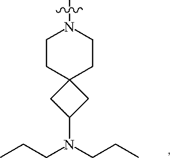 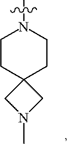 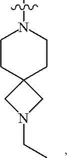 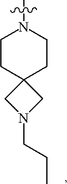 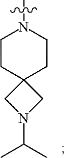
5) 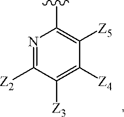
wherein Z
₂, Z₃, Z₄, Z₅ each are independently selected from H, C₁—C₆ alkyl, C₁—C₆ alkoxy, piperazinyl, N-methylpiperazinyl, N-ethylpiperazinyl, N-n-propylpiperazinyl, or N-isopropylpiperazinyl, and Z₂, Z₃, Z₄, Z₅ are not hydrogen at the same time;
6) 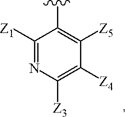
wherein Z
₁, Z₃, Z₄, Z₅ each are independently selected from H, C₁—C₆ alkyl, C₁—C₆ alkoxy, N-(N-methyl-3-tetrahydropyrrolyl)amino, N-methyl-N-(N-methyl-3-tetrahydropyrrolyl)amino, N-methylpiperazinyl, N-ethylpiperazinyl, N-n-propylpiperazinyl, N-isopropylpiperazinyl, 4-(N-methylpiperazinyl-1-)piperidyl,4-(N-ethylpiperazinyl-1-)piperidyl, or 4-(N-isopropylpiperazinyl-1-)piperidyl, and Z₁, Z₃, Z₄, Z₅ are not hydrogen at the same time;
7) 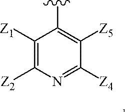
wherein Z
₁, Z₂, Z₄, Z₅ each are independently selected from H, or C₁—C₆ alkyl, and Z₁, Z₂, Z₄, Z₅ may be hydrogen at the same time;
8) 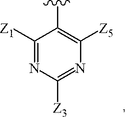
wherein Z
₁, Z₃, Z₅ each are independently selected from H, C₁—C₆ alkyl, C₁—C₆ alkoxy, N-methylpiperazinyl, N-ethylpiperazinyl, N-n-propylpiperazinyl, or N-isopropylpiperazinyl, and Z₁, Z₃, Z₅ are not hydrogen at the same time;
9) 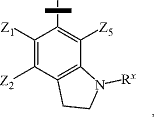
wherein Z
₁, Z₂, Z₅ each are independently selected from H, C₁—C₆ alkyl, or C₁—C₆ alkoxy, and Z₁, Z₂, Z₅ may be hydrogen at the same time, R^{x} is selected from aminoformyl, methylaminoformyl, N,N-dimethylaminoformyl, ethylaminoformyl, aminoacetyl, methylaminoacetyl, 2-(N,N-dimethylamino)acetyl, or ethylaminoacetyl;
10) 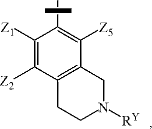
wherein Z
₁, Z₂, Z₅ each are independently selected from H, F, Cl, Br, I, C₁—C₆ alkyl, C₁—C₆ alkoxy, C₁—C₆ fluorine-containing alkyl, or C₁—C₆ fluorine-containing alkoxy, and Z₁, Z₂, Z₅ may be hydrogen at the same time, R^{y} is C₁—C₆ alkyl;
11) 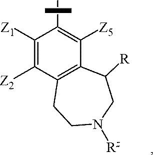
wherein Z
₁, Z₂, Z₅ each are independently selected from H, or C₁—C₆ alkyl, and Z₁, Z₂, Z₅ may be hydrogen at the same time, R^{z}, R each are independently selected from C₁—C₆ alkyl;
most preferably, R¹ is selected from:
1) cyclopropyl, 2-N,N-dimethylaminoethyl, 2-hydroxyethyl, N-methyl-4-piperidyl, 4-aminocyclohexyl, 1-methyl-3-pyrazolyl, 1-ethyl-3-pyrazolyl, 1-isopropyl-3-pyrazolyl, 1,5-dimethyl-3-pyrazolyl, 1-methyl-4-pyrazolyl, 1-ethyl-4-pyrazolyl, 1-isopropyl-4-pyrazolyl, 1-ethyl-3-methyl-4-pyrazolyl, 1,3-dimethyl-5-pyrazolyl, 1-methyl-4-imidazolyl;
2) 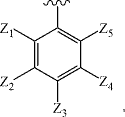
wherein:
Z₁, Z₂, Z₃, Z₄, Z₅ each are independently selected from the following groups, and Z₁, Z₂, Z₃, Z₄, Z₅ are not hydrogen at the same time:
H, F, 2-hydroxyethylamino, 2-N,N-dimethylaminoethylamino, N-methyl-N-(2-N,N-dimethylamino)ethylamino, methyl, ethyl, isopropyl, cyclopropyl, methoxy, ethoxy, isopropoxy, trifluoromethyl, trifluoromethoxy, vinyl, piperazin-1-yl, N-methylpiperazinyl, 4-(N-methylpiperidyl-4-)piperazinyl, 4-(azetidin-3-yl)piperazinyl, morpholinyl, 3-(N,N-dimethylamino)tetrahydropyrrolyl, 4-N,N-dimethylaminopiperidyl, 4-hydroxypiperidyl, 4-cyanopiperidyl, 4-(N-methylpiperazinyl-1-)piperidyl, 4-(piperazinyl-1-)piperidyl, 4-(tetrahydropyrrolyl-1-)piperidyl, N-methyl-4-piperidyl, 2-(N,N-dimethylamino)acetamido, cyclopropylsulfamido, N-methyl-N-(2-N,N-dimethylamino)ethylamino, 4-methyl-1-piperazinylmethylene, benzyl, cyclopropylmethylene, 4-(N-methyl-1-piperazinyl)piperidyl-1-formyl, N-methylpiperazinyl-1-formyl, cyclopropylaminosulfonyl, N-methylpiperazinyl-1-sulfonyl, 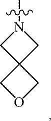 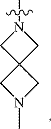 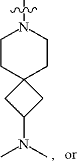 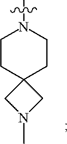
preferably, Z
₁, Z₂, Z₅ each are independently selected from H, F, methoxy, ethoxy, isopropoxy, trifluoromethyl, or trifluoromethoxy; and one of Z₃ and Z₄ is selected from the following groups, and the other is H, F, methyl, ethyl, isopropyl, cyclopropyl, vinyl, benzyl or cyclopropylmethylene:
F, 2-hydroxyethylamino, 2-N,N-dimethylaminoethylamino, N-methyl-N-(2-N,N-dimethylamino)ethylamino, piperazin-1-yl, N-methylpiperazinyl, 4-(N-methylpiperidyl-4-)piperazinyl, 4-(azetidin-3-yl)piperazinyl, morpholinyl, 3-(N,N-dimethylamino)tetrahydropyrrolyl, 4-N, N-dimethylaminopiperidyl, 4-hydroxypiperidyl, 4-cyanopiperidyl, 4-(N-methylpiperazinyl-1-)piperidyl, 4-(piperazinyl-1-)piperidyl, 4-(tetrahydropyrrolyl-1-)piperidyl, N-methyl-4-piperidyl, 2-(N,N-dimethylamino)acetamido, cyclopropylsulfamido, N-methyl-N-(2-N,N-dimethylamino)ethylamino, 4-methyl-1-piperazinylmethylene, 4-(N-methyl-1-piperazinyl)piperidyl-1-formyl, N-methylpiperazinyl-1-formyl, cyclopropylaminosulfonyl, N-methylpiperazinyl-1-sulfonyl, 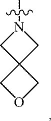 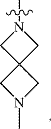 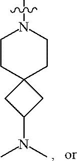 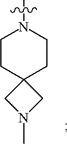
3) 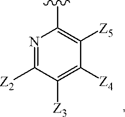
wherein Z
₂, Z₃, Z₄, Z₅ each are independently selected from H, methoxy, or N-methylpiperazinyl, and Z₂, Z₃, Z₄, Z₅ are not hydrogen at the same time;
4) 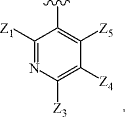
wherein:
Z₁, Z₃, Z₄, Z₅ each are independently selected from H, methyl, ethyl, methoxy, N-(N-methyl-3-tetrahydropyrrolyl)amino,
N-methyl-N-(N-methyl-3-tetrahydropyrrolyl)amino, N-methylpiperazinyl, or 4-(N-methylpiperazinyl-1-)piperidyl, and Z₁, Z₃, Z₄, Z₅ are not hydrogen at the same time; preferably, Z₁, Z₄, Z₅ each are independently selected from H, methyl, ethyl, or methoxy, Z₃ is methyl, N-(N-methyl-3-tetrahydropyrrolyl)amino, N-methyl-N-(N-methyl-3-tetrahydropyrrolyl)amino, N-methylpiperazinyl or 4-(N-methylpiperazinyl-1-)piperidyl;
5) 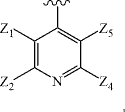
wherein Z
₁, Z₂, Z₄, Z₅ are H;
6) 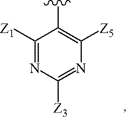
wherein Z
₁, Z₃, Z₅ each are independently selected from H, methoxy, or N-methylpiperazinyl, and Z₁, Z₃, Z₅ are not hydrogen at the same time;
7) 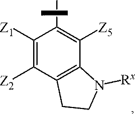
wherein Z
₁, Z₂, Z₅ each are independently selected from H, or methoxy, R^{x} is 2-(N,N-dimethylamino)acetyl;
8) 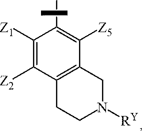
wherein Z
₁, Z₂, Z₅ each are independently selected from H, F, Cl, methyl, methoxy, trifluoromethyl, or trifluoromethoxy, R^{y} is methyl;
9) 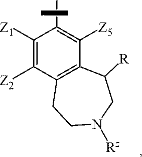
wherein Z
₁, Z₂, Z₅ are H, R^{z}, R are methyl;
R², R³ each are independently selected from H, C₁—C₆ alkyl, C₁—C₆ fluorine-containing alkyl, C₃—C₆ cycloalkyl, or C₃—C₆ fluorine-containing cycloalkyl;
preferably, R², R³ each are independently selected from H, C₁—C₆ alkyl, C₁—C₆ fluorine-containing alkyl, or C₃—C₆ cycloalkyl;
more preferably, R², R³ each are independently selected from H, methyl, trifluoromethyl, ethyl, isopropyl, or cyclopropyl;
most preferably, R² is selected from methyl, trifluoromethyl, ethyl, isopropyl, or cyclopropyl, R³ is selected from H, methyl, ethyl, isopropyl, or cyclopropyl;
R⁵ is selected from H, F, Cl, Br, C₁—C₆ fluorine-containing alkyl, cyano, nitro, C₁—C₆ alkyl, C₁—C₆ alkoxy, or C₃—C₆ cycloalkyl;
preferably, R⁵ is selected from H, F, Cl, Br, trifluoromethyl, cyano, nitro, methyl, ethyl, isopropyl, methoxy, ethoxy, or cyclopropyl.

3. The compound according to claim 1, having the following formula: 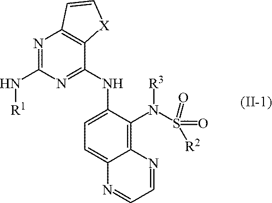 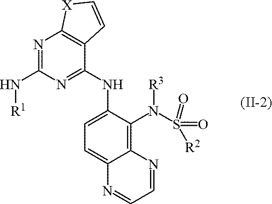
or a stereoisomer, a prodrug, a pharmaceutically acceptable salt or a pharmaceutically acceptable solvate thereof, wherein:
each X is independently selected from NH, O, or S;
each R¹ is independently selected from:
1) 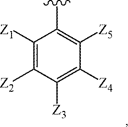
wherein Z
₁, Z₂, Z₃, Z₄, Z₅ each are independently selected from the following groups, and Z₁, Z₂, Z₃, Z₄, Z₅ are not hydrogen at the same time:
(1) H, F, Cl, Br, I, nitro, cyano,
(2) C₁—C₆ alkyl, C₁—C₆ alkoxy, C₁—C₆ oxygen-containing alkyl, C₁—C₆ fluorine-containing alkyl, C₁—C₆ fluorine-containing alkoxy, C₃—C₆ cycloalkyl, C₂—C₆ alkenyl,
(3) 2-N,N-dimethylaminoethoxy, 3-N,N-dimethylaminopropoxy, 2-(N-methylpiperazinyl)ethoxy, 2-(N-acetylpiperazinyl)ethoxy, 2-morpholinylethoxy, 2-thiomorpholinylethoxy, 2-piperidylethoxy, 3-N,N-dimethylaminopropoxy, 3-N,N-diethylaminopropoxy, 3-N,N-diisopropylaminopropoxy, 3-(N-methylpiperazinyl)propoxy, 3-(N-acetylpiperazinyl)propoxy, 3-morpholinylpropoxy, 3-thiomorpholinylpropoxy, 3-piperidylpropoxy, 2-pyridylmethoxy, 3-pyridylmethoxy, 4-pyridylmethoxy, phenylmethoxy, monohalo-substituted phenylmethoxy, gem-dihalo-substituted phenylmethoxy, hetero-dihalo-substituted phenylmethoxy,
(4) piperazinyl, N-methylpiperazinyl, N-ethylpiperazinyl, N-n-propylpiperazinyl, N-isopropylpiperazinyl, N-cyclopropylpiperazinyl, N-(2-hydroxyethyl)piperazinyl, N-(2-N,N-dimethylaminoethyl)piperazinyl, N-(2-N,N-diethylaminoethyl)piperazinyl, N-(2-cyanoethyl)piperazinyl, N-cyanomethylpiperazinyl, N-(3-hydroxypropyl)piperazinyl, N-(3-N,N-dimethylaminopropyl)piperazinyl, N-(3-N,N-diethylaminopropyl)piperazinyl, N-(N-methyl-4-piperidyl)piperazinyl, N-(N-ethyl-4-piperidyl)piperazinyl, N-t-butoxycarbonylpiperazinyl, N-acetylpiperazinyl, N-propionylpiperazinyl, N-isobutyrylpiperazinyl, N-cyclopropylformylpiperazinyl, N-methylsulfonylpiperazinyl, N-ethylsulfonylpiperazinyl, N-n-propylsulfonylpiperazinyl, N-isopropylsulfonylpiperazinyl, N-cyclopropylsulfonylpiperazinyl, 2-oxo-piperazin-4-yl,
(5) morpholinyl, 2,6-dimethylmorpholinyl, thiomorpholinyl,
(6) tetrahydropyrrolyl, 3-(N,N-dimethylamino)tetrahydropyrrolyl, 3-(N,N-diethylamino)tetrahydropyrrolyl,
(7) imidazolyl, 4-methyl-1-imidazolyl,
(8) piperidyl, 4-N,N-dimethylaminopiperidyl, 4-N,N-diethylaminopiperidyl, 4-N,N-diisopropylaminopiperidyl, 4-hydroxypiperidyl, 4-methoxypiperidyl, 4-ethoxypiperidyl, 4-(N-methylpiperazinyl-1-)piperidyl, 4-(N-ethylpiperazinyl-1-)piperidyl, 4-(N-isopropylpiperazinyl-1-)piperidyl, 4-(N-acetylpiperazinyl-1-)piperidyl, 4-(N-t-butoxycarbonylpiperazinyl-1-)piperidyl, 4-(N-methylsulfonylpiperazinyl-1-)piperidyl, 4-(N-(2-hydroxyethyl)piperazinyl-1-)piperidyl, 4-(N-(2-cyanoethyl)piperazinyl-1-)piperidyl, 4-(N-(3-hydroxypropyl)piperazinyl-1-)piperidyl, 4-(N-(2-N,N-dimethylaminoethyl)piperazinyl-1 -)piperidyl, 4-(N-(2-N,N-diethylethyl)piperazinyl-1 -)piperidyl, 4-(N-(3-N,N-dimethylpropyl)piperazinyl-1-)piperidyl, 4-(N-(3-N,N-diethylpropyl)piperazinyl-1-)piperidyl, 4-(tetrahydropyrrolyl-1-)piperidyl, 4-(3-N,N-dimethylaminotetrahydropyrrolyl-1-)piperidyl, 4-(3-N,N-diethylaminotetrahydropyrrolyl-1-)piperidyl,
(9) piperidin-4-yl, N-methyl-4-piperidyl, N-ethyl-4-piperidyl, N-isopropyl-4-piperidyl, N-acetyl-4-piperidyl, N-t-butoxycarbonyl-4-piperidyl, N-methylsulfonyl-4-piperidyl, N-(2-hydroxyethyl)-4-piperidyl, N-(2-N,N-dimethylaminoethyl)-4-piperidyl, N-(2-N,N-diethylaminoethyl)-4-piperidyl, N-(2-cyanoethyl)-4-piperidyl, N-(3-hydroxypropyl)-4-piperidyl, N-(3-N,N-dimethylaminopropyl)-4-piperidyl, N-(3-N,N-diethylaminopropyl)-4-piperidyl, N-(3-cyanopropyl)-4-piperidyl, N-cyanomethylene-4-piperidyl,
(10) amino, N,N-dimethylamino, N,N-diethylamino, N,N-di-n-propylamino, N,N-diisopropylamino, 2-N,N-dimethylaminoethylamino, 2-morpholinylethylamino, 2-(N-methylpiperazinyl)ethylamino, 3-N,N-dimethylaminopropylamino, 3-N,N-diethylaminopropylamino, 3-N,N-diisopropylaminopropylamino, 3-morpholinylpropylamino, 3-(N-methylpiperazinyl)propylamino, N-methylpiperidyl-4-amino, N-ethylpiperidyl-4-amino, N-n-propylpiperidyl-4-amino, N-isopropylpiperidyl-4-amino, acetamido, propionamido, 2-(N,N-dimethylamino)acetamido, 2-hydroxyacetamido, 2-methoxyacetamido, methylsulfamido, ethylsulfamido, n-propylsulfamido, isopropylsulfamido, cyclopropylsulfamido, N-methyl-N-(N-methyl-3-tetrahydropyrrolyl)amino, N-(N-methyl-3-tetrahydropyrrolyl)amino, N-methyl-N-(2-N,N-dimethylamino)ethylamino,
(11) aminomethylene, N,N-dimethylaminomethylene, N,N-diethylaminomethylene, 4-methyl-1-piperazinylmethylene, 4-ethyl-1-piperazinylmethylene, 4-propyl-1-piperazinylmethylene, 4-isopropyl-1-piperazinylmethylene, 4-(2-hydroxyethyl)-1-piperazinylmethylene, 4-(2-N,N-dimethylaminoethyl)-1-piperazinylmethylene, piperidyl-1-methylene, 4-(N,N-dimethylamino)-1-piperidylmethylene, 4-hydroxypiperidyl-1-methylene, tetrahydropyrrolyl-1-methylene, 3-(N,N-dimethylamino)-1-tetrahydropyrrolylmethylene, benzyl, cyclopropylmethylene, cyclobutylmethylene, cyclopentylmethylene, cyclohexylmethylene,
(12) aminoformyl, methylaminoformyl, N,N-dimethylaminoformyl, ethylaminoformyl, cyclopropylaminoformyl, cyclobutylaminoformyl, cyclopentylaminoformyl, cyclohexylaminoformyl, aminoacetyl, methylaminoacetyl, 2-(N,N-dimethylamino)acetyl, ethylaminoacetyl, cyclopropylaminoacetyl, cyclobutylaminoacetyl, cyclopentylaminoacetyl, cyclohexylaminoacetyl, tetrahydropyrrolyl-1-formyl, 3-(N,N-dimethylamino)tetrahydropyrrolyl-1-formyl, 3-(N,N-diethylamino)tetrahydropyrrolyl-1-formyl, morpholinyl-4-formyl, thiomorpholinyl-4-formyl, 2,6-dimethylmorpholinyl-4-formyl, piperidyl-1-formyl, 4-hydroxypiperidyl-1-formyl, 4-(N,N-dimethylamino)piperidyl-1-formyl, 4-(N,N-diethylamino)piperidyl-1-formyl, 4-(N-methyl-1-piperazinyl)piperidyl-1-formyl, 4-(N-ethyl-1-piperazinyl)piperidyl-1-formyl, N-methylpiperazinyl-1-formyl, N-ethylpiperazinyl-1-formyl, N-isopropylpiperazinyl-1-formyl, N-(2-hydroxyethyl)piperazinyl-1-formyl, N-(2-N,N-dimethylaminoethyl)piperazinyl-1-formyl, N-(2-N,N-diethylaminoethyl)piperazinyl-1-formyl, 4-(N-methyl-4-piperidyl)piperazinyl-1-formyl, 4-(N-ethyl-4-piperidyl)piperazinyl-1-formyl,
(13) hydroxyformyl, methoxyformyl, ethoxyformyl, propoxyformyl, isopropoxyformyl, n-butoxyformyl, iso-butoxyformyl, t-butoxyformyl,
(14) hydroxysulfonyl, aminosulfonyl, methylaminosulfonyl, ethylaminosulfonyl, propylaminosulfonyl, isopropylaminosulfonyl, cyclopropylaminosulfonyl, cyclobutylaminosulfonyl, cyclopentylaminosulfonyl, cyclohexylaminosulfonyl, tetrahydropyrrolyl-1-sulfonyl, 3-(N,N-dimethylamino)tetrahydropyrrolyl-1-sulfonyl, 3-(N,N-diethylamino)tetrahydropyrrolyl-1-sulfonyl, morpholinyl-4-sulfonyl, thiomorpholinyl-4-sulfonyl, 2,6-dimethylmorpholinyl-4-sulfonyl, piperidyl-1-sulfonyl, 4-hydroxypiperidyl-1-sulfonyl, 4-(N,N-dimethylamino)piperidyl-1-sulfonyl, 4-(N,N-diethylamino)piperidyl-1-sulfonyl, 4-(N-methyl-1-piperazinyl)piperidyl-1-sulfonyl, 4-(N-ethyl-1-piperazinyl)piperidyl-1-sulfonyl, N-methylpiperazinyl-1-sulfonyl, N-ethylpiperazinyl-1-sulfonyl, N-isopropylpiperazinyl-1-sulfonyl, N-(2-hydroxyethyl)piperazinyl-1-sulfonyl, N-(2-N,N-dimethylaminoethyl)piperazinyl-1-sulfonyl, N-(2-N,N-diethylaminoethyl)piperazinyl-1-sulfonyl, 4-(N-methyl-4-piperidyl)piperazinyl-1-sulfonyl, 4-(N-ethyl-4-piperidyl)piperazinyl-1-sulfonyl,
(15) aminoformamido, methylaminoformamido, ethylaminoformamido, propylaminoformamido, isopropylaminoformamido, cyclopropylaminoformamido, cyclobutylaminoformamido, cyclopentylaminoformamido, piperidyl-1-formamido, 4-hydroxypiperidyl-1-formamido, 4-N,N-dimethylpiperidyl-1-formamido, 4-N,N-diethylpiperidyl-1-formamido, tetrahydropyrrolyl-1-formamido, 3-N,N-dimethyltetrahydropyrrolyl-1-formamido, 3-N,N-diethyltetrahydropyrrolyl-1-formamido, N-methylpiperazinyl-1-formamido, N-ethylpiperazinyl-1-formamido, N-acetylpiperazinyl-1-formam ido, N-t-butoxycarbonylpiperazinyl-1-formamido, N-(2-hydroxyethyl)piperazinyl-1-formamido, N-(2-cyanoethyl)piperazinyl-1-formam ido, N-(2-N,N-dimethylaminoethyl)piperazinyl-1-formamido, N-(2-N,N-diethylaminoethyl)piperazinyl-1-formamido, N-(3-hydroxypropyl)piperazinyl-1-formamido, N-(3-N,N-dimethylpropyl)piperazinyl-1 -formamido, N-(3-N,N-diethylpropyl)piperazinyl-1-formamido, morpholinyl-1-formamido, 3,5-dimethylmorpholinyl-1-formamido, 4-(tetrahydropyrrolyl)piperidyl-1-formamido, 4-(N-methyl-1-piperazinyl)piperidyl-1-formamido, 4-(N-ethyl-1-piperazinyl)piperidyl-1-formamido, 4-(N-acetyl-1-piperazinyl)piperidyl-1-formamido, N-(N-methyl-4-piperidyl)piperazinyl-1-formamido;
2) 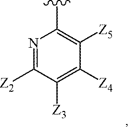
wherein Z
₂, Z₃, Z₄, Z₅ are the same as defined in 1), and Z₂, Z₃, Z₄, Z₅ are not hydrogen at the same time;
3) 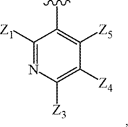
wherein Z
₁, Z₃, Z₄, Z₅ are the same as defined in 1), and Z₁, Z₃, Z₄, Z₅ are not hydrogen at the same time;
4) 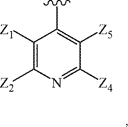
wherein Z
₁, Z₂, Z₄, Z₅ are the same as defined in 1), and Z₁, Z₂, Z₄, Z₅ may be hydrogen at the same time;
5) 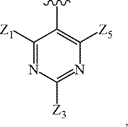
wherein Z
₁, Z₃, Z₅ are the same as defined in 1), and Z₁, Z₃, Z₅ are not hydrogen at the same time;
preferably, each R¹ is independently selected from:
1) 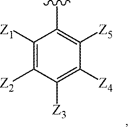
wherein Z
₁, Z₂, Z₃, Z₄, Z₅ each are independently selected from the following groups, and Z₁, Z₂, Z₃, Z₄, Z₅ are not hydrogen at the same time:
(1) H, F, Cl, Br, I, nitro, cyano,
(2) C₁—C₆ alkyl, C₁—C₆ alkoxy, C₁—C₆ oxygen-containing alkyl, C₁—C₆ fluorine-containing alkyl, C₁—C₆ fluorine-containing alkoxy, C₃—C₆ cycloalkyl, C₂—C₆ alkenyl,
(3) piperazinyl, N-methylpiperazinyl, N-ethylpiperazinyl, N-n-propylpiperazinyl, N-isopropylpiperazinyl, N-cyclopropylpiperazinyl, N-(2-hydroxyethyl)piperazinyl, N-(2-N,N-dimethylaminoethyl)piperazinyl, N-(2-N,N-diethylaminoethyl)piperazinyl, N-(2-cyanoethyl)piperazinyl, N-cyanomethylpiperazinyl, N-(3-hydroxypropyl)piperazinyl, N-(3-N,N-dimethylaminopropyl)piperazinyl, N-(3-N,N-diethylaminopropyl)piperazinyl,
(4) morpholinyl, 2,6-dimethylmorpholinyl, thiomorpholinyl,
(5) tetrahydropyrrolyl, 3-(N,N-dimethylamino)tetrahydropyrrolyl, 3-(N,N-diethylamino)tetrahydropyrrolyl,
(6) imidazolyl, 4-methyl-1-imidazolyl,
(7) piperidyl, 4-N,N-dimethylaminopiperidyl, 4-N,N-diethylaminopiperidyl, 4-N,N-diisopropylaminopiperidyl, 4-hydroxypiperidyl, 4-methoxypiperidyl, 4-ethoxypiperidyl, 4-(N-methylpiperazinyl-1-)piperidyl, 4-(N-ethylpiperazinyl-1-)piperidyl, 4-(N-isopropylpiperazinyl-1-)piperidyl, 4-(N-(2-hydroxyethyl)piperazinyl-1-)piperidyl, 4-(N-(2-cyanoethyl)piperazinyl-1-)piperidyl, 4-(N-(3-hydroxypropyl)piperazinyl-1-)piperidyl, 4-(N-(2-N,N-dimethylaminoethyl)piperazinyl-1-)piperidyl, 4-(N-(2-N,N-diethylethyl)piperazinyl-1 -)piperidyl, 4-(N-(3-N,N-dimethylpropyl)piperazinyl-1-)piperidyl, 4-(N-(3-N,N-diethylpropyl)piperazinyl-1-)piperidyl, 4-(tetrahydropyrrolyl-1-)piperidyl, 4-(3-N,N-dimethylaminotetrahydropyrrolyl-1-)piperidyl, 4-(3-N,N-diethylaminotetrahydropyrrolyl-1-)piperidyl,
(8) piperidin-4-yl, N-methyl-4-piperidyl, N-ethyl-4-piperidyl, N-isopropyl-4-piperidyl, N-(2-hydroxyethyl)-4-piperidyl, N-(2-N,N-dimethylaminoethyl)-4-piperidyl, N-(2-N,N-diethylaminoethyl)-4-piperidyl, N-(2-cyanoethyl)-4-piperidyl, N-(3-hydroxypropyl)-4-piperidyl, N-(3-N,N-dimethylaminopropyl)-4-piperidyl, N-(3-N,N-diethylaminopropyl)-4-piperidyl, N-(3-cyanopropyl)-4-piperidyl, N-cyanomethylene-4-piperidyl,
(9) amino, N,N-dimethylamino, N,N-diethylamino, N,N-di-n-propylamino, N,N-diisopropylamino, 2-N, N-dimethylaminoethylamino, 3-N,N-dimethylaminopropylamino, 3-N,N-diethylaminopropylamino, 3-N,N-diisopropylaminopropylamino, N-methylpiperidyl-4-amino, N-ethylpiperidyl-4-amino, N-n-propylpiperidyl-4-amino, N-isopropylpiperidyl-4-amino, acetamido, propionamido, 2-(N,N-dimethylamino)acetamido, 2-hydroxyacetamido, 2-methoxyacetamido, methylsulfamido, ethylsulfamido, n-propylsulfamido, isopropylsulfamido, cyclopropylsulfamido, N-methyl-N-(N-methyl-3-tetrahydropyrrolyl)am ino, N-(N-methyl-3-tetrahydropyrrolyl)amino, N-methyl-N-(2-N,N-dimethylamino)ethylamino,
(10) 4-methyl-1-piperazinylmethylene, 4-ethyl-1-piperazinylmethylene, 4-propyl-1-piperazinylmethylene, 4-isopropyl-1-piperazinylmethylene, 4-(2-hydroxyethyl)-1-piperazinylmethylene, 4-(2-N,N-dimethylaminoethyl)-1-piperazinylmethylene, piperidyl-1-methylene, 4-(N,N-dimethylamino)-1-piperidylmethylene, 4-hydroxypiperidyl-1-methylene, tetrahydropyrrolyl-1-methylene, 3-(N,N-dimethylamino)-1-tetrahydropyrrolylmethylene, benzyl, cyclopropylmethylene, cyclobutylmethylene, cyclopentylmethylene, cyclohexylmethylene,
(11) aminoformyl, methylaminoformyl, N,N-dimethylaminoformyl, ethylaminoformyl, aminoacetyl, methylaminoacetyl, 2-(N,N-dimethylamino)acetyl, ethylaminoacetyl, tetrahydropyrrolyl-1-formyl, 3-(N,N-dimethylamino)tetrahydropyrrolyl-1-formyl, 3-(N,N-diethylamino)tetrahydropyrrolyl-1-formyl, morpholinyl-4-formyl, thiomorpholinyl-4-formyl, 2,6-dimethylmorpholinyl-4-formyl, piperidyl-1-formyl, 4-hydroxypiperidyl-1-formyl, 4-(N,N-dimethylamino)piperidyl-1-formyl, 4-(N,N-diethylamino)piperidyl-1-formyl, 4-(N-methyl-1-piperazinyl)piperidyl-1-formyl, 4-(N-ethyl-1-piperazinyl)piperidyl-1-formyl, N-methylpiperazinyl-1-formyl, N-ethylpiperazinyl-1-formyl, N-isopropylpiperazinyl-1-formyl, N-(2-hydroxyethyl)piperazinyl-1-formyl, N-(2-N,N-dimethylaminoethyl)piperazinyl-1-formyl, N-(2-N,N-diethylaminoethyl)piperazinyl-1-formyl, 4-(N-methyl-4-piperidyl)piperazinyl-1-formyl, 4-(N-ethyl-4-piperidyl)piperazinyl-1-formyl,
(12) hydroxysulfonyl, aminosulfonyl, methylaminosulfonyl, ethylaminosulfonyl, propylaminosulfonyl, isopropylaminosulfonyl, cyclopropylaminosulfonyl, cyclobutylaminosulfonyl, cyclopentylaminosulfonyl, cyclohexylaminosulfonyl, tetrahydropyrrolyl-1-sulfonyl, 3-(N,N-dimethylamino)tetrahydropyrrolyl-1-sulfonyl, 3-(N,N-diethylamino)tetrahydropyrrolyl-1-sulfonyl, morpholinyl-4-sulfonyl, thiomorpholinyl-4-sulfonyl, 2,6-dimethylmorpholinyl-4-sulfonyl, piperidyl-1-sulfonyl, 4-hydroxypiperidyl-1-sulfonyl, 4-(N,N-dimethylamino)piperidyl-1-sulfonyl, 4-(N,N-diethylamino)piperidyl-1-sulfonyl, N-methylpiperazinyl-1-sulfonyl, N-ethylpiperazinyl-1-sulfonyl, N-isopropylpiperazinyl-1-sulfonyl, N-(2-hydroxyethyl)piperazinyl-1-sulfonyl, N-(2-N,N-dimethylaminoethyl)piperazinyl-1-sulfonyl, N-(2-N,N-diethylaminoethyl)piperazinyl-1-sulfonyl,
(13) aminoformamido, methylaminoformamido, ethylaminoformamido, propylaminoformamido, isopropylaminoformamido;
2) 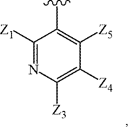
wherein Z
₁, Z₃, Z₄, Z₅ are the same as defined in 1), and Z₁, Z₃, Z₄, Z₅ are not hydrogen at the same time;
more preferably, each R¹ is independently selected from:
1) 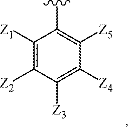
wherein Z
₁, Z₂, Z₃, Z₄, Z₅ each are independently selected from the following groups, and Z₁, Z₂, Z₃, Z₄, Z₅ are not hydrogen at the same time:
(1) H, F, Cl, Br, I, nitro, cyano,
(2) C₁—C₆ alkyl, C₁—C₆ alkoxy, C₁—C₆ fluorine-containing alkoxy, C₂—C₆ alkenyl,
(3) piperazinyl, N-methylpiperazinyl, N-ethylpiperazinyl, N-n-propylpiperazinyl, N-isopropylpiperazinyl,
(5) 3-(N,N-dimethylamino)tetrahydropyrrolyl, 3-(N,N-diethylamino)tetrahydropyrrolyl,
(6) 4-N,N-dimethylaminopiperidyl, 4-N,N-diethylaminopiperidyl, 4-N,N-diisopropylaminopiperidyl, 4-(N-methylpiperazinyl-1-)piperidyl, 4-(N-ethylpiperazinyl-1-)piperidyl, 4-(N-isopropylpiperazinyl-1-)piperidyl, 4-hydroxypiperidyl,
(7) benzyl, cyclopropylmethylene, cyclobutylmethylene, cyclopentylmethylene, cyclohexylmethylene;
2) 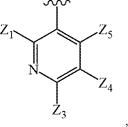
wherein Z
₁, Z₃, Z₄, Z₅ each are independently selected from H, C₁—C₆ alkyl, C₁—C₆ alkoxy, 4-(N-methylpiperazinyl-1-)piperidyl, 4-(N-ethylpiperazinyl-1-)piperidyl, or 4-(N-isopropylpiperazinyl-1-)piperidyl, and Z₁, Z₃, Z₄, Z₅ are not hydrogen at the same time;
most preferably, each R¹ is independently selected from:
1) 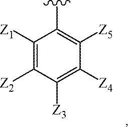
wherein:
Z₁, Z₂, Z₃, Z₄, Z₅ each are independently selected from the following groups, and Z₁, Z₂, Z₃, Z₄, Z₅ are not hydrogen at the same time:
H, methyl, ethyl, methoxy, trifluoromethoxy, vinyl, N-methylpiperazinyl, 3-(N,N-dimethylamino)tetrahydropyrrolyl, 4-N,N-dimethylaminopiperidyl, 4-(N-methylpiperazinyl-1-)piperidyl, 4-hydroxypiperidyl, benzyl, or cyclopropylmethylene; preferably, Z₁, Z₂, Z₅ each are independently selected from H, methoxy, trifluoromethoxy, Z₄ is H, methyl, ethyl, vinyl, benzyl, cyclopropylmethylene or 4-hydroxypiperidyl, Z₃ is H, N-methylpiperazinyl, 3-(N,N-dimethylamino)tetrahydropyrrolyl, 4-N,N-dimethylamino piperidyl or 4-(N-methylpiperazinyl-1-)piperidyl, Z₁, Z₂, Z₃, Z₄, Z₅ are not hydrogen at the same time;
2) 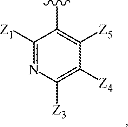
wherein:
Z₁, Z₃, Z₄, Z₅ each are independently selected from H, methyl, ethyl, methoxy, or 4-(N-methylpiperazinyl-1-)piperidyl, and Z₁, Z₃, Z₄, Z₅ are not hydrogen at the same time; preferably, Z₁, Z₄, Z₅ each are independently selected from H, methyl, ethyl, or methoxy, Z₃ is 4-(N-methylpiperazinyl-1-)piperidyl;
R², R³ each are independently selected from:
H, C₁—C₆ alkyl, C₁—C₆ fluorine-containing alkyl, C₃—C₆ cycloalkyl, or C₃—C₆ fluorine-containing cycloalkyl;
preferably, R², R³ each are independently selected from H, or C₁—C₆ alkyl;
more preferably, R², R³ each are independently selected from methyl.

4. The compound according to claim 1, which is selected from:

| | | | |
|---|---|---|---|
| No. | Structure | No. | Structure |
| I-1 | 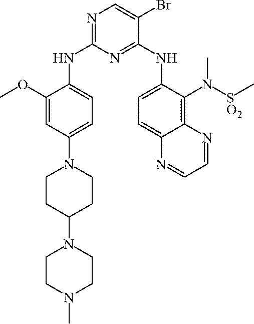 | I-106 | 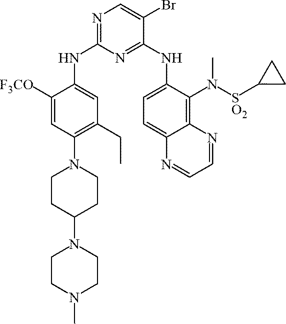 [SMILES: CCc1cc(Nc2ncc(Br)c(Nc3ccc4nccnc4c3N(C)S(=O)(=O)C3CC3)n2)c(OC(F)(F)F)cc1N1CCC(N2CCN(C)CC2)CC1] |
| I-2 | 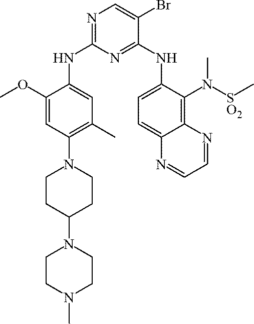 | I-107 | 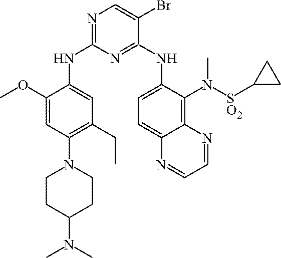 [SMILES: CCc1cc(Nc2ncc(Br)c(Nc3ccc4nccnc4c3N(C)S(=O)(=O)C3CC3)n2)c(OC)cc1N1CCC(N(C)C)CC1] |
| I-3 | 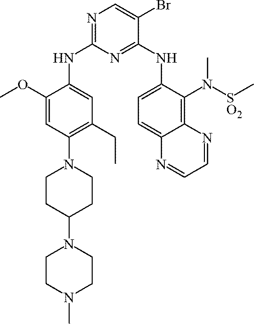 | I-108 | 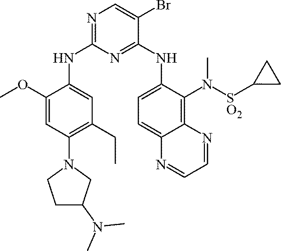 [SMILES: CCc1cc(Nc2ncc(Br)c(Nc3ccc4nccnc4c3N(C)S(=O)(=O)C3CC3)n2)c(OC)cc1N1CCC(N(C)C)C1] |
| I-4 | 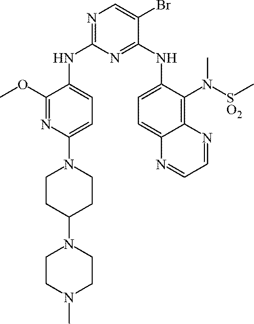 | I-109 | 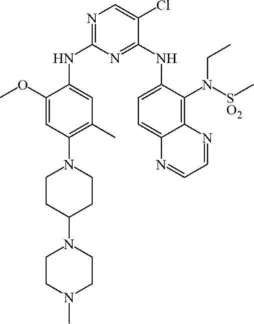 |
| I-5 | 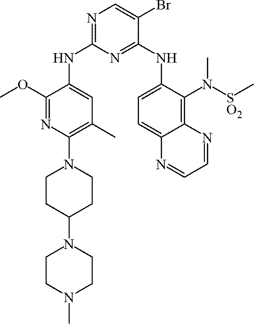 | I-110 | 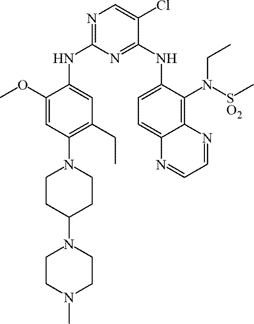 |
| I-6 | 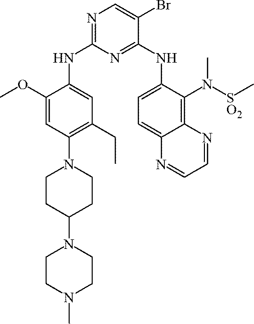 | I-111 | 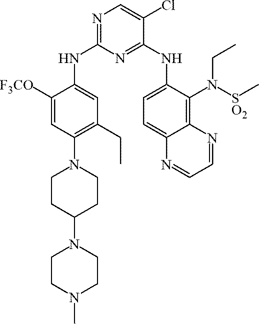 [SMILES: CCc1cc(Nc2ncc(Cl)c(Nc3ccc4nccnc4c3N(CC)S(C)(=O)=O)n2)c(OC(F)(F)F)cc1N1CCC(N2CCN(C)CC2)CC1] |
| I-7 | 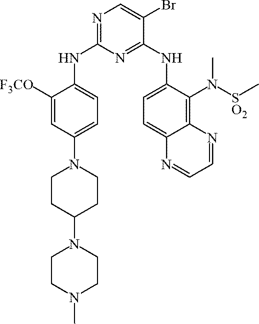 [SMILES: CN1CCN(C2CCN(c3ccc(Nc4ncc(Br)c(Nc5ccc6nccnc6c5N(C)S(C)(=O)=O)n4)c(OC(F)(F)F)c3)CC2)CC1] | I-112 | 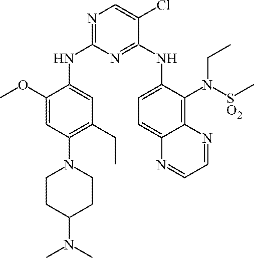 |
| I-8 | 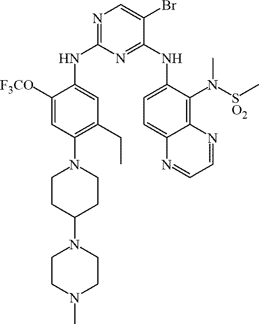 [SMILES: CCc1cc(Nc2ncc(Br)c(Nc3ccc4nccnc4c3N(C)S(C)(=O)=O)n2)c(OC(F)(F)F)cc1N1CCC(N2CCN(C)CC2)CC1] | I-113 | 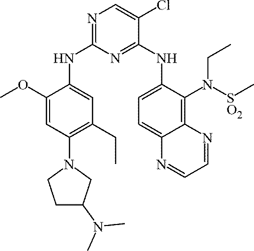 |
| I-9 | 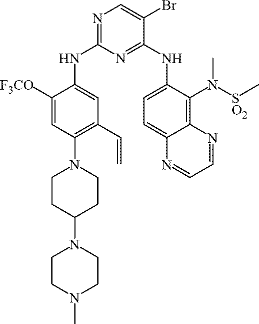 [SMILES: C=Cc1cc(Nc2ncc(Br)c(Nc3ccc4nccnc4c3N(C)S(C)(=O)=O)n2)c(OC(F)(F)F)cc1N1CCC(N2CCN(C)CC2)CC1] | I-114 | 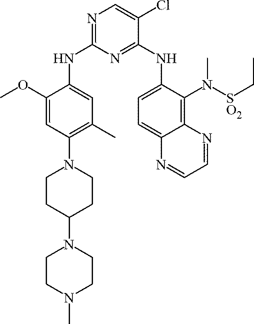 [SMILES: CCS(=O)(=O)N(C)c1c(Nc2nc(Nc3cc(C)c(N4CCC(N5CCN(C)CC5)CC4)cc3OC)ncc2Cl)ccc2nccnc12] |
| I-10 | 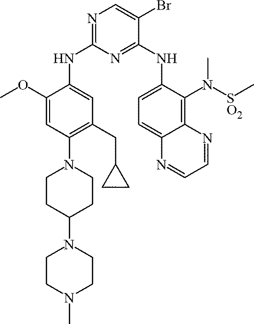 | I-115 | 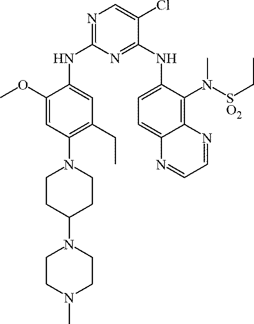 [SMILES: CCc1cc(Nc2ncc(Cl)c(Nc3ccc4nccnc4c3N(C)S(=O)(=O)CC)n2)c(OC)cc1N1CCC(N2CCN(C)CC2)CC1] |
| I-11 | 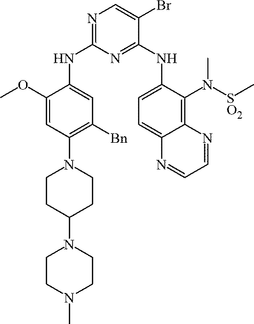 | I-116 | 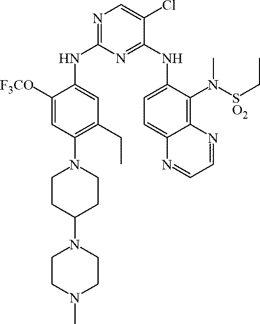 [SMILES: CCc1cc(Nc2ncc(Cl)c(Nc3ccc4nccnc4c3N(C)S(=O)(=O)CC)n2)c(OC(F)(F)F)cc1N1CCC(N2CCN(C)CC2)CC1] |
| I-12 | 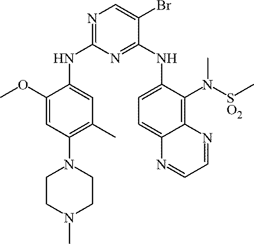 | I-117 | 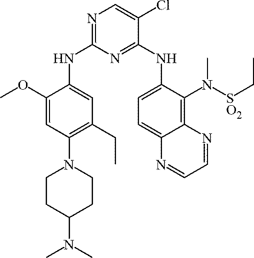 [SMILES: CCc1cc(Nc2ncc(Cl)c(Nc3ccc4nccnc4c3N(C)S(=O)(=O)CC)n2)c(OC)cc1N1CCC(N(C)C)CC1] |
| I-13 | 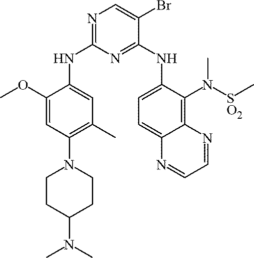 | I-118 | 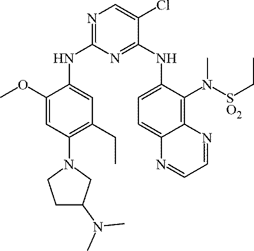 [SMILES: CCc1cc(Nc2ncc(Cl)c(Nc3ccc4nccnc4c3N(C)S(=O)(=O)CC)n2)c(OC)cc1N1CCC(N(C)C)C1] |
| I-14 | 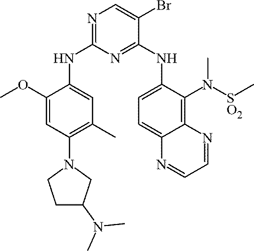 | I-119 | 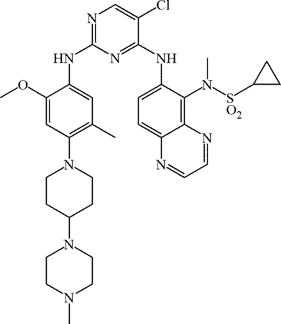 [SMILES: COc1cc(N2CCC(N3CCN(C)CC3)CC2)c(C)cc1Nc1ncc(Cl)c(Nc2ccc3nccnc3c2N(C)S(=O)(=O)C2CC2)n1] |
| I-15 | 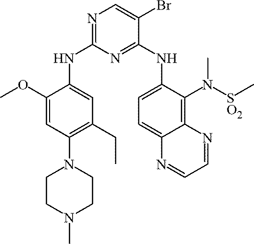 | I-120 | 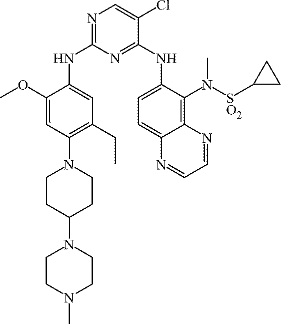 [SMILES: CCc1cc(Nc2ncc(Cl)c(Nc3ccc4nccnc4c3N(C)S(=O)(=O)C3CC3)n2)c(OC)cc1N1CCC(N2CCN(C)CC2)CC1] |
| I-16 | 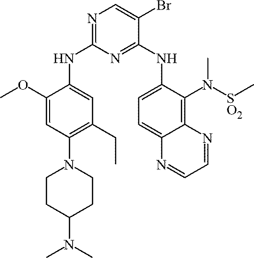 | I-121 | 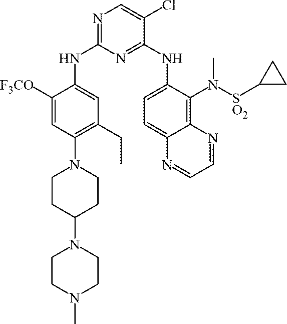 [SMILES: CCc1cc(Nc2ncc(Cl)c(Nc3ccc4nccnc4c3N(C)S(=O)(=O)C3CC3)n2)c(OC(F)(F)F)cc1N1CCC(N2CCN(C)CC2)CC1] |
| I-17 | 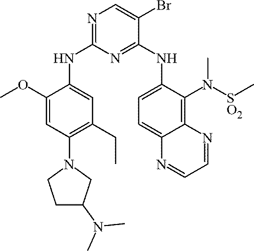 | I-122 | 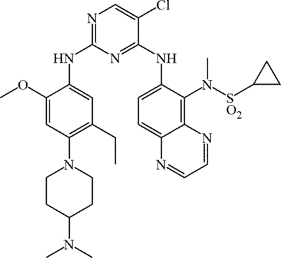 [SMILES: CCc1cc(Nc2ncc(Cl)c(Nc3ccc4nccnc4c3N(C)S(=O)(=O)C3CC3)n2)c(OC)cc1N1CCC(N(C)C)CC1] |
| I-18 | 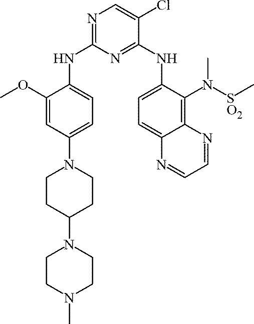 | I-123 | 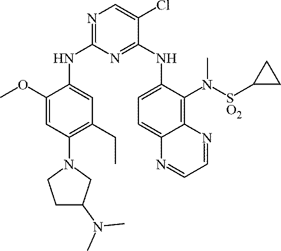 [SMILES: CCc1cc(Nc2ncc(Cl)c(Nc3ccc4nccnc4c3N(C)S(=O)(=O)C3CC3)n2)c(OC)cc1N1CCC(N(C)C)C1] |
| I-19 | 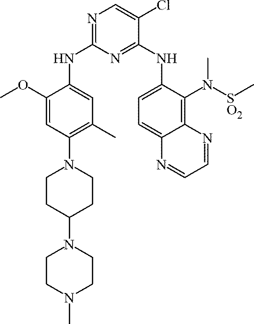 | I-124 | 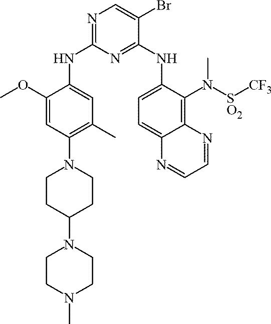 [SMILES: COc1cc(N2CCC(N3CCN(C)CC3)CC2)c(C)cc1Nc1ncc(Br)c(Nc2ccc3nccnc3c2N(C)S(=O)(=O)C(F)(F)F)n1] |
| I-20 | 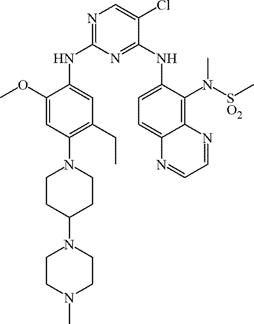 | I-125 | 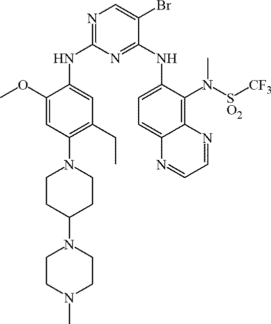 [SMILES: CCc1cc(Nc2ncc(Br)c(Nc3ccc4nccnc4c3N(C)S(=O)(=O)C(F)(F)F)n2)c(OC)cc1N1CCC(N2CCN(C)CC2)CC1] |
| I-21 | 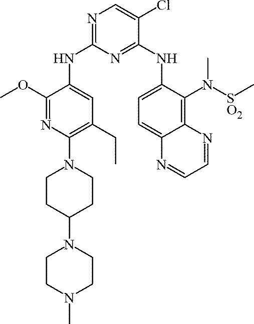 | I-126 | 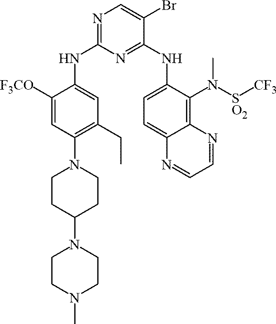 [SMILES: CCc1cc(Nc2ncc(Br)c(Nc3ccc4nccnc4c3N(C)S(=O)(=O)C(F)(F)F)n2)c(OC(F)(F)F)cc1N1CCC(N2CCN(C)CC2)CC1] |
| I-22 | 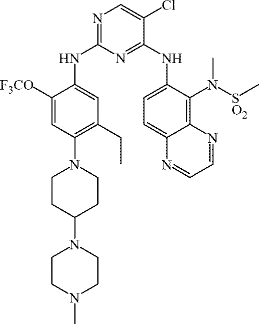 [SMILES: CCc1cc(Nc2ncc(Cl)c(Nc3ccc4nccnc4c3N(C)S(C)(=O)=O)n2)c(OC(F)(F)F)cc1N1CCC(N2CCN(C)CC2)CC1] | I-127 | 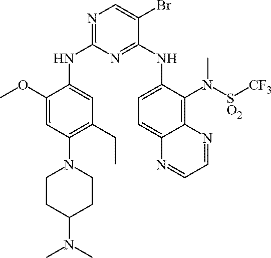 [SMILES: CCc1cc(Nc2ncc(Br)c(Nc3ccc4nccnc4c3N(C)S(=O)(=O)C(F)(F)F)n2)c(OC)cc1N1CCC(N(C)C)CC1] |
| I-23 | 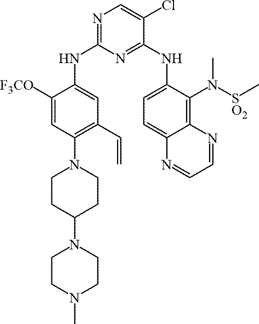 [SMILES: C=Cc1cc(Nc2ncc(Cl)c(Nc3ccc4nccnc4c3N(C)S(C)(=O)=O)n2)c(OC(F)(F)F)cc1N1CCC(N2CCN(C)CC2)CC1] | I-128 | 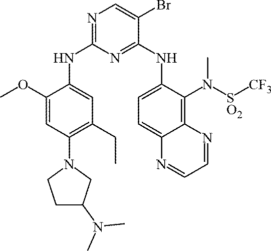 [SMILES: CCc1cc(Nc2ncc(Br)c(Nc3ccc4nccnc4c3N(C)S(=O)(=O)C(F)(F)F)n2)c(OC)cc1N1CCC(N(C)C)C1] |
| I-24 | 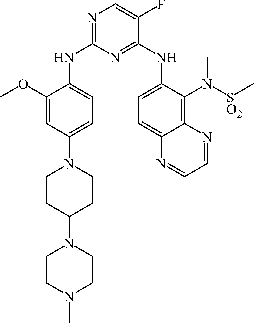 | I-129 | 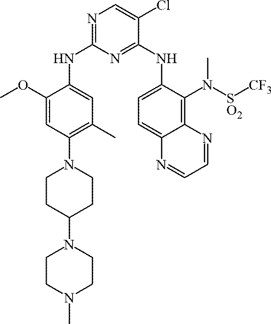 [SMILES: COc1cc(N2CCC(N3CCN(C)CC3)CC2)c(C)cc1Nc1ncc(Cl)c(Nc2ccc3nccnc3c2N(C)S(=O)(=O)C(F)(F)F)n1] |
| I-25 | 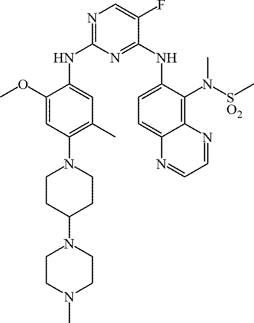 | I-130 | 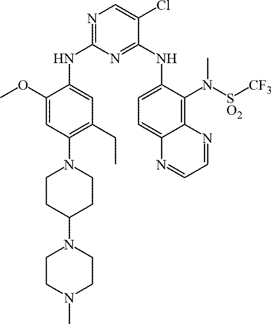 [SMILES: CCc1cc(Nc2ncc(Cl)c(Nc3ccc4nccnc4c3N(C)S(=O)(=O)C(F)(F)F)n2)c(OC)cc1N1CCC(N2CCN(C)CC2)CC1] |
| I-26 | 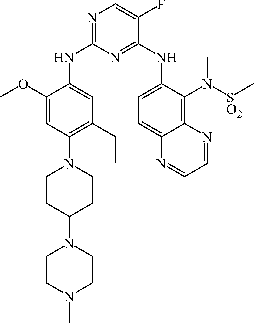 | I-131 | 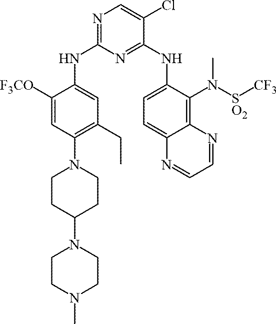 [SMILES: CCc1cc(Nc2ncc(Cl)c(Nc3ccc4nccnc4c3N(C)S(=O)(=O)C(F)(F)F)n2)c(OC(F)(F)F)cc1N1CCC(N2CCN(C)CC2)CC1] |
| I-27 | 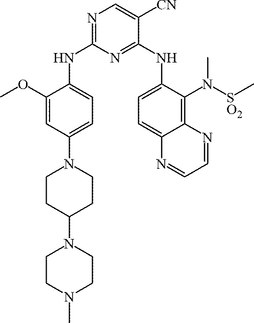 | I-132 | 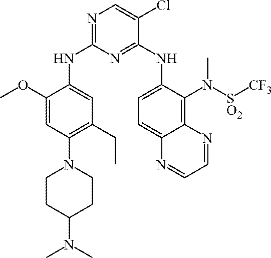 [SMILES: CCc1cc(Nc2ncc(Cl)c(Nc3ccc4nccnc4c3N(C)S(=O)(=O)C(F)(F)F)n2)c(OC)cc1N1CCC(N(C)C)CC1] |
| I-28 | 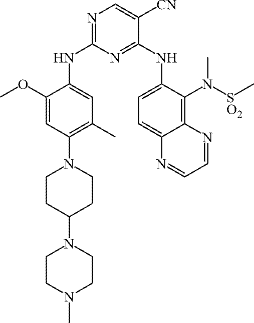 | I-133 | 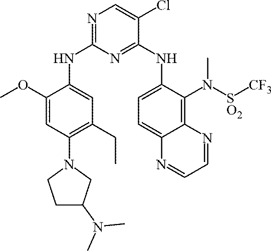 [SMILES: CCc1cc(Nc2ncc(Cl)c(Nc3ccc4nccnc4c3N(C)S(=O)(=O)C(F)(F)F)n2)c(OC)cc1N1CCC(N(C)C)C1] |
| I-29 | 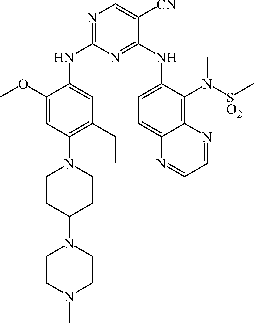 | I-134 | 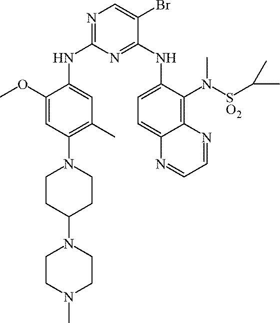 [SMILES: COc1cc(N2CCC(N3CCN(C)CC3)CC2)c(C)cc1Nc1ncc(Br)c(Nc2ccc3nccnc3c2N(C)S(=O)(=O)C(C)C)n1] |
| I-30 | 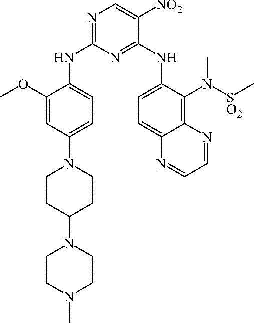 | I-135 | 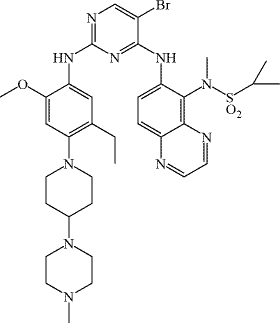 [SMILES: CCc1cc(Nc2ncc(Br)c(Nc3ccc4nccnc4c3N(C)S(=O)(=O)C(C)C)n2)c(OC)cc1N1CCC(N2CCN(C)CC2)CC1] |
| I-31 | 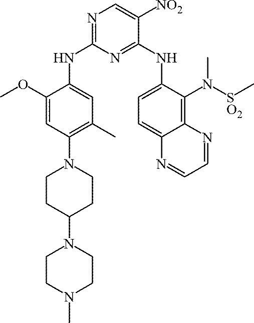 | I-136 | 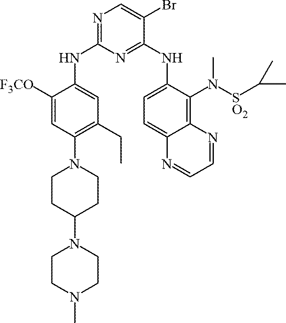 [SMILES: CCc1cc(Nc2ncc(Br)c(Nc3ccc4nccnc4c3N(C)S(=O)(=O)C(C)C)n2)c(OC(F)(F)F)cc1N1CCC(N2CCN(C)CC2)CC1] |
| I-32 | 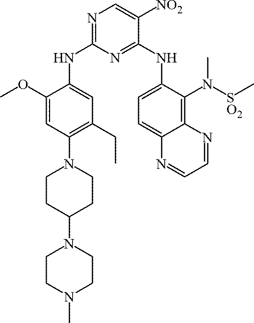 | I-137 | 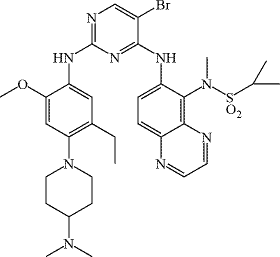 [SMILES: CCc1cc(Nc2ncc(Br)c(Nc3ccc4nccnc4c3N(C)S(=O)(=O)C(C)C)n2)c(OC)cc1N1CCC(N(C)C)CC1] |
| I-33 | 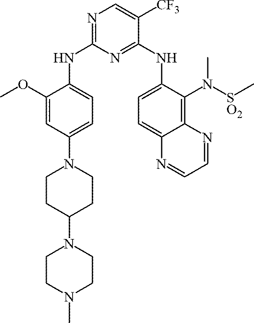 | I-138 | 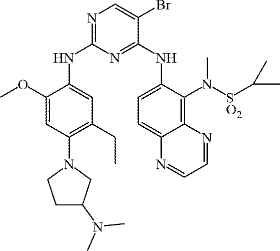 [SMILES: CCc1cc(Nc2ncc(Br)c(Nc3ccc4nccnc4c3N(C)S(=O)(=O)C(C)C)n2)c(OC)cc1N1CCC(N(C)C)C1] |
| I-34 | 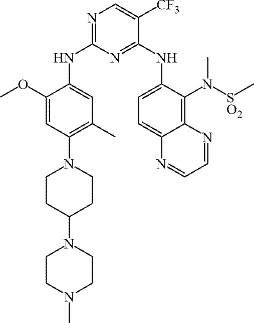 | I-139 | 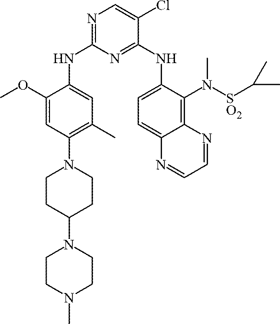 [SMILES: COc1cc(N2CCC(N3CCN(C)CC3)CC2)c(C)cc1Nc1ncc(Cl)c(Nc2ccc3nccnc3c2N(C)S(=O)(=O)C(C)C)n1] |
| I-35 | 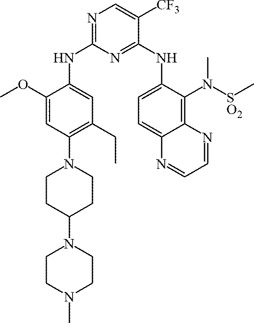 | I-140 | 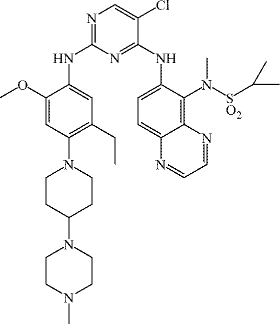 [SMILES: CCc1cc(Nc2ncc(Cl)c(Nc3ccc4nccnc4c3N(C)S(=O)(=O)C(C)C)n2)c(OC)cc1N1CCC(N2CCN(C)CC2)CC1] |
| I-36 | 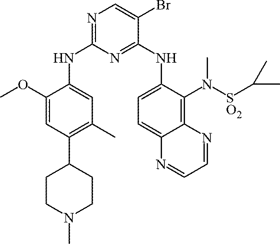 [SMILES: COc1cc(C2CCN(C)CC2)c(C)cc1Nc1ncc(Br)c(Nc2ccc3nccnc3c2N(C)S(=O)(=O)C(C)C)n1] | I-141 | 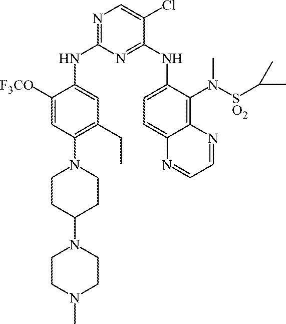 [SMILES: CCc1cc(Nc2ncc(Cl)c(Nc3ccc4nccnc4c3N(C)S(=O)(=O)C(C)C)n2)c(OC(F)(F)F)cc1N1CCC(N2CCN(C)CC2)CC1] |
| I-37 | 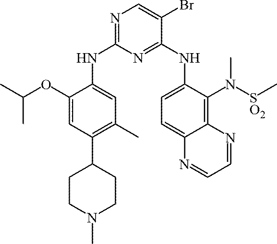 [SMILES: Cc1cc(Nc2ncc(Br)c(Nc3ccc4nccnc4c3N(C)S(C)(=O)=O)n2)c(OC(C)C)cc1C1CCN(C)CC1] | I-142 | 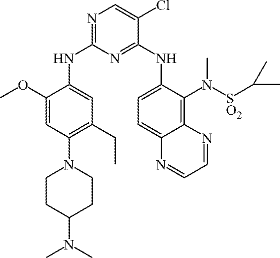 [SMILES: CCc1cc(Nc2ncc(Cl)c(Nc3ccc4nccnc4c3N(C)S(=O)(=O)C(C)C)n2)c(OC)cc1N1CCC(N(C)C)CC1] |
| I-38 | 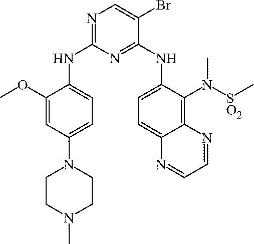 | I-143 | 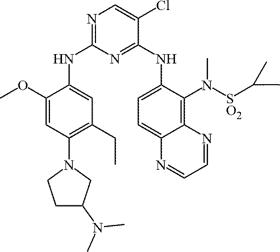 [SMILES: CCc1cc(Nc2ncc(Cl)c(Nc3ccc4nccnc4c3N(C)S(=O)(=O)C(C)C)n2)c(OC)cc1N1CCC(N(C)C)C1] |
| I-39 | 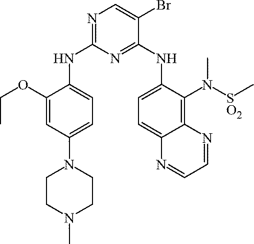 [SMILES: CCOc1cc(N2CCN(C)CC2)ccc1Nc1ncc(Br)c(Nc2ccc3nccnc3c2N(C)S(C)(=O)=O)n1] | I-144 | 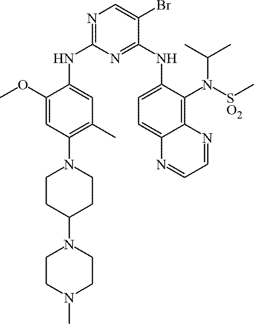 |
| I-40 | 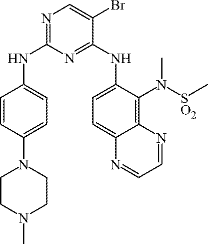 [SMILES: CN1CCN(c2ccc(Nc3ncc(Br)c(Nc4ccc5nccnc5c4N(C)S(C)(=O)=O)n3)cc2)CC1] | I-145 | 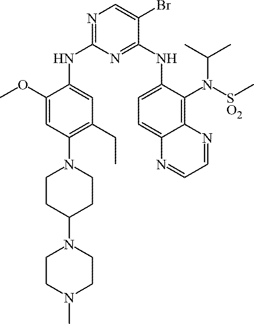 |
| I-41 | 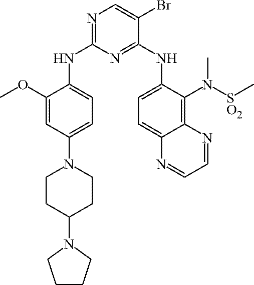 | I-146 | 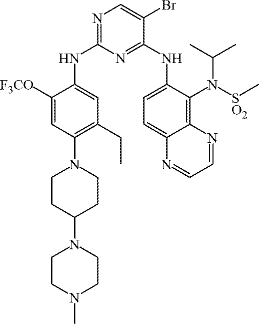 [SMILES: CCc1cc(Nc2ncc(Br)c(Nc3ccc4nccnc4c3N(C(C)C)S(C)(=O)=O)n2)c(OC(F)(F)F)cc1N1CCC(N2CCN(C)CC2)CC1] |
| I-42 | 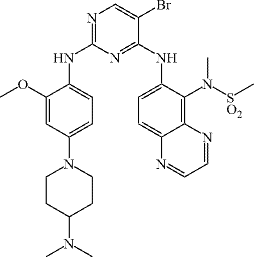 | I-147 | 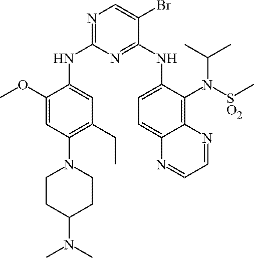 |
| I-43 | 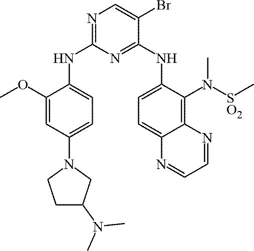 | I-148 | 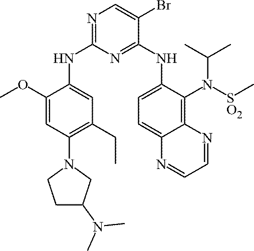 |
| I-44 | 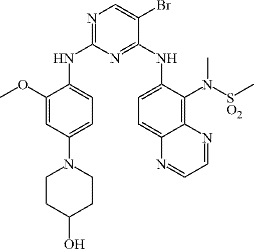 | I-149 | 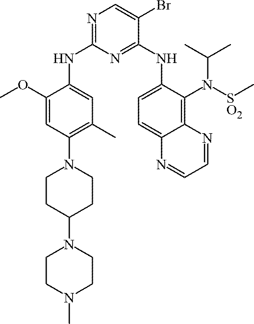 |
| I-45 | 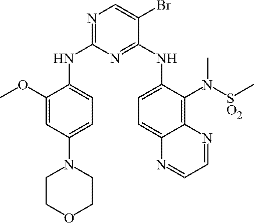 | I-150 | 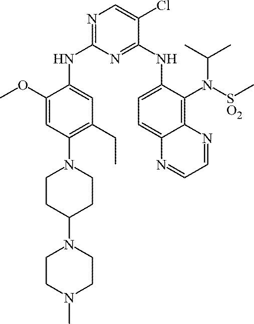 |
| I-46 | 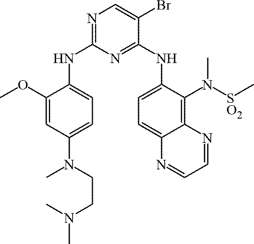 | I-151 | 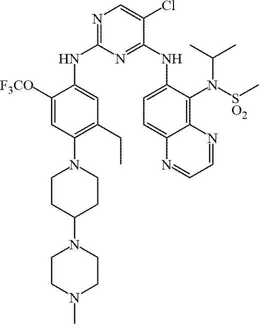 [SMILES: CCc1cc(Nc2ncc(Cl)c(Nc3ccc4nccnc4c3N(C(C)C)S(C)(=O)=O)n2)c(OC(F)(F)F)cc1N1CCC(N2CCN(C)CC2)CC1] |
| I-47 | 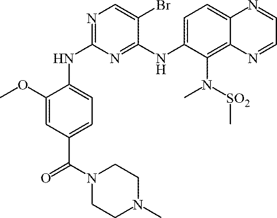 [SMILES: COc1cc(C(=O)N2CCN(C)CC2)ccc1Nc1ncc(Br)c(Nc2ccc3nccnc3c2N(C)S(C)(=O)=O)n1] | I-152 | 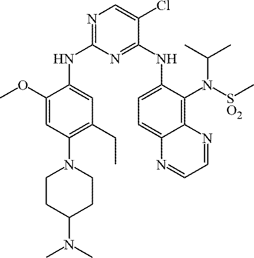 |
| I-48 | 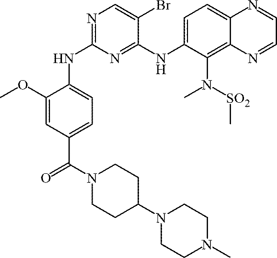 [SMILES: COc1cc(C(=O)N2CCC(N3CCN(C)CC3)CC2)ccc1Nc1ncc(Br)c(Nc2ccc3nccnc3c2N(C)S(C)(=O)=O)n1] | I-153 | 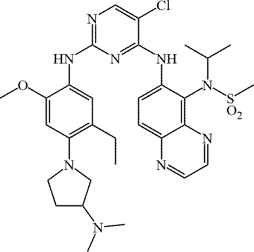 |
| I-49 | 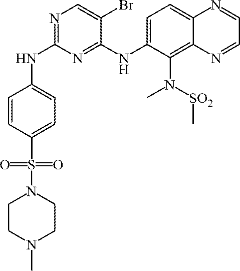 [SMILES: CN1CCN(S(=O)(=O)c2ccc(Nc3ncc(Br)c(Nc4ccc5nccnc5c4N(C)S(C)(=O)=O)n3)cc2)CC1] | I-154 | 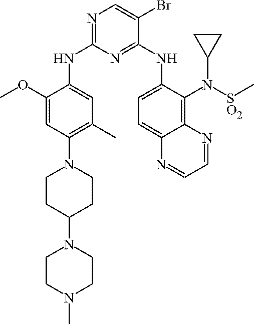 |
| I-50 | 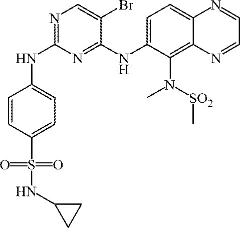 [SMILES: CN(c1c(Nc2nc(Nc3ccc(S(=O)(=O)NC4CC4)cc3)ncc2Br)ccc2nccnc12)S(C)(=O)=O] | I-155 | 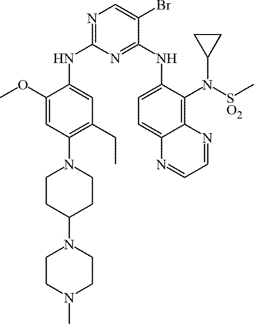 |
| I-51 | 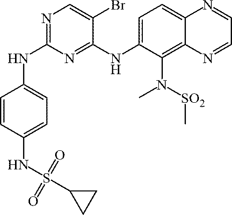 [SMILES: CN(c1c(Nc2nc(Nc3ccc(NS(=O)(=O)C4CC4)cc3)ncc2Br)ccc2nccnc12)S(C)(=O)=O] | I-156 | 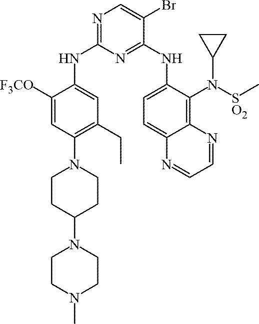 [SMILES: CCc1cc(Nc2ncc(Br)c(Nc3ccc4nccnc4c3N(C3CC3)S(C)(=O)=O)n2)c(OC(F)(F)F)cc1N1CCC(N2CCN(C)CC2)CC1] |
| I-52 | 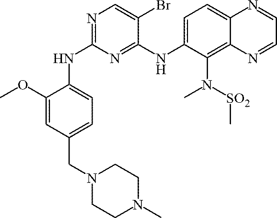 [SMILES: COc1cc(CN2CCN(C)CC2)ccc1Nc1ncc(Br)c(Nc2ccc3nccnc3c2N(C)S(C)(=O)=O)n1] | I-157 | 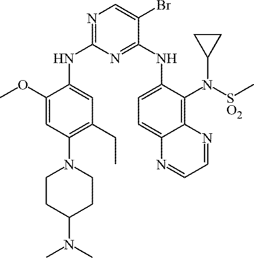 |
| I-53 | 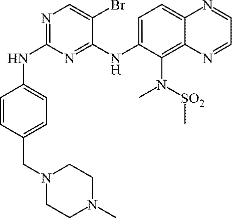 [SMILES: CN1CCN(Cc2ccc(Nc3ncc(Br)c(Nc4ccc5nccnc5c4N(C)S(C)(=O)=O)n3)cc2)CC1] | I-158 | 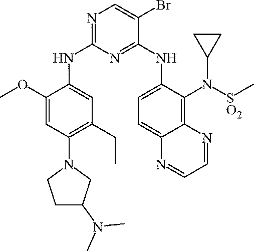 |
| I-54 | 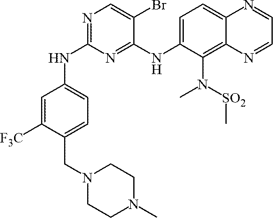 [SMILES: CN1CCN(Cc2ccc(Nc3ncc(Br)c(Nc4ccc5nccnc5c4N(C)S(C)(=O)=O)n3)cc2C(F)(F)F)CC1] | I-159 | 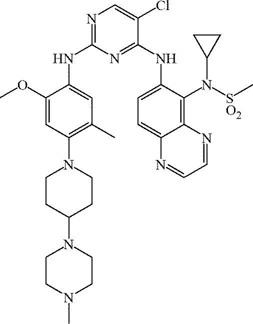 |
| I-55 | 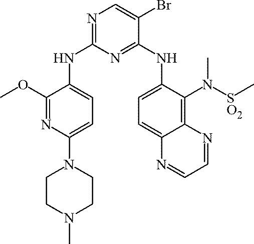 | I-160 | 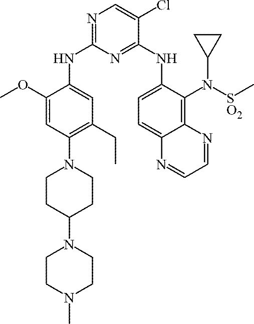 |
| I-56 | 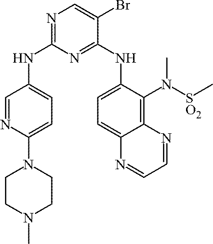 [SMILES: CN1CCN(c2ccc(Nc3ncc(Br)c(Nc4ccc5nccnc5c4N(C)S(C)(=O)=O)n3)cn2)CC1] | I-161 | 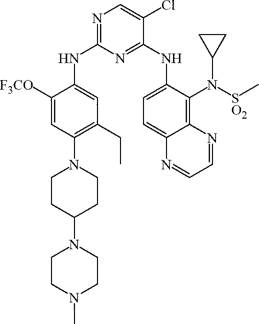 [SMILES: CCc1cc(Nc2ncc(Cl)c(Nc3ccc4nccnc4c3N(C3CC3)S(C)(=O)=O)n2)c(OC(F)(F)F)cc1N1CCC(N2CCN(C)CC2)CC1] |
| I-57 | 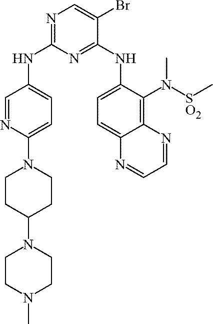 [SMILES: CN1CCN(C2CCN(c3ccc(Nc4ncc(Br)c(Nc5ccc6nccnc6c5N(C)S(C)(=O)=O)n4)cn3)CC2)CC1] | I-162 | 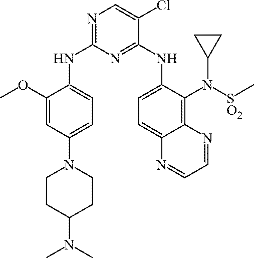 |
| I-58 | 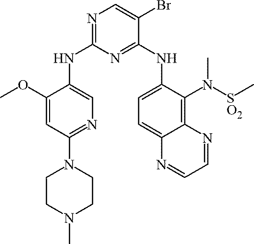 | I-163 | 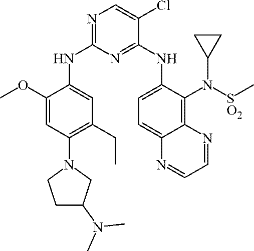 |
| I-59 | 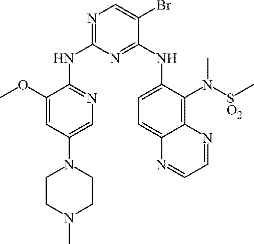 | II-1-1 | 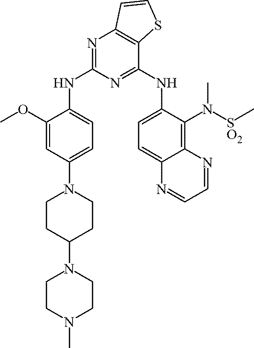 |
| I-60 | 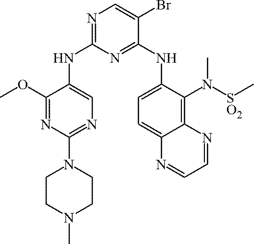 | II-1-2 | 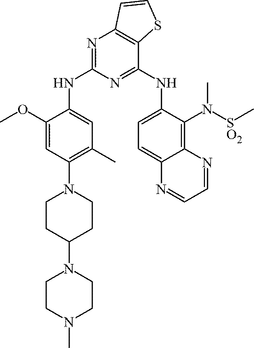 |
| I-61 | 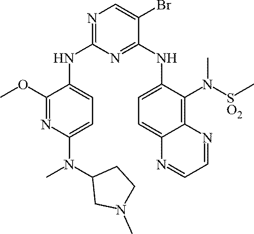 | II-1-3 | 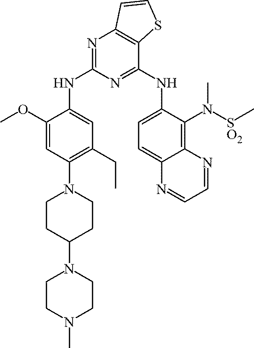 |
| I-62 | 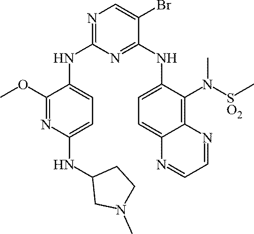 | II-1-4 | 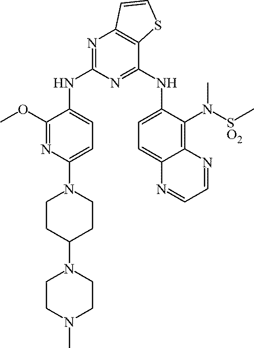 |
| I-63 | 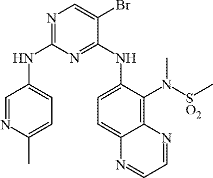 [SMILES: Cc1ccc(Nc2ncc(Br)c(Nc3ccc4nccnc4c3N(C)S(C)(=O)=O)n2)cn1] | II-1-5 | 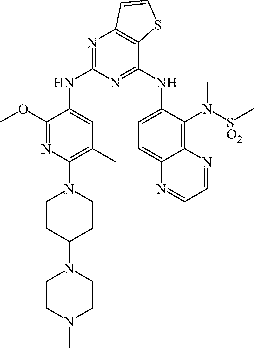 |
| I-64 | 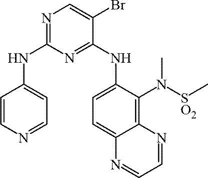 [SMILES: CN(c1c(Nc2nc(Nc3ccncc3)ncc2Br)ccc2nccnc12)S(C)(=O)=O] | II-1-6 | 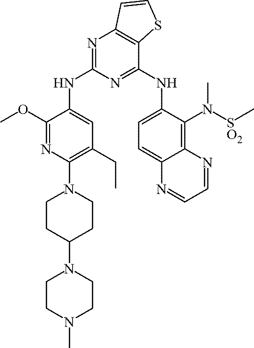 |
| I-65 | 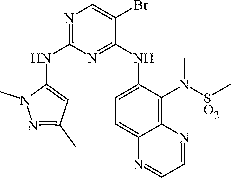 | II-1-7 | 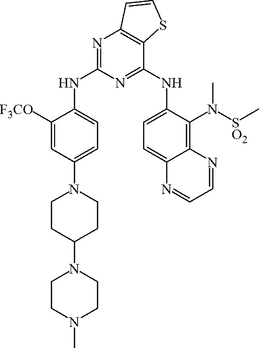 [SMILES: CN1CCN(C2CCN(c3ccc(Nc4nc(Nc5ccc6nccnc6c5N(C)S(C)(=O)=O)c5sccc5n4)c(OC(F)(F)F)c3)CC2)CC1] |
| I-66 | 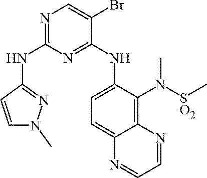 [SMILES: CN(c1c(Nc2nc(Nc3ccn(C)n3)ncc2Br)ccc2nccnc12)S(C)(=O)=O] | II-1-8 | 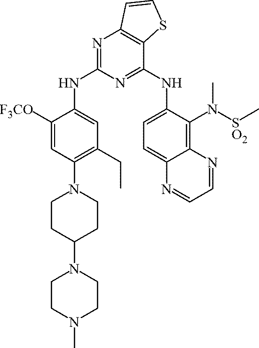 [SMILES: CCc1cc(Nc2nc(Nc3ccc4nccnc4c3N(C)S(C)(=O)=O)c3sccc3n2)c(OC(F)(F)F)cc1N1CCC(N2CCN(C)CC2)CC1] |
| I-67 | 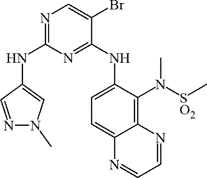 [SMILES: CN(c1c(Nc2nc(Nc3cnn(C)c3)ncc2Br)ccc2nccnc12)S(C)(=O)=O] | II-1-9 | 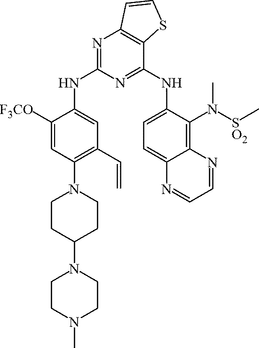 [SMILES: C=Cc1cc(Nc2nc(Nc3ccc4nccnc4c3N(C)S(C)(=O)=O)c3sccc3n2)c(OC(F)(F)F)cc1N1CCC(N2CCN(C)CC2)CC1] |
| I-68 | 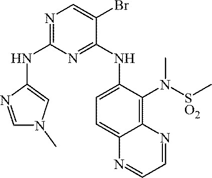 [SMILES: CN(c1c(Nc2nc(Nc3cn(C)cn3)ncc2Br)ccc2nccnc12)S(C)(=O)=O] | II-1-10 | 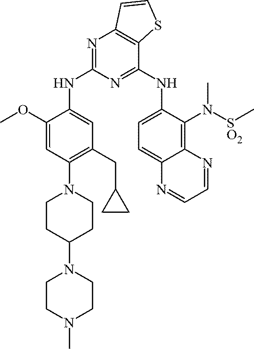 |
| I-69 | 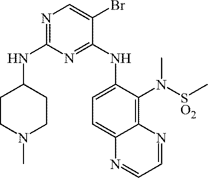 [SMILES: CN1CCC(Nc2ncc(Br)c(Nc3ccc4nccnc4c3N(C)S(C)(=O)=O)n2)CC1] | II-1-11 | 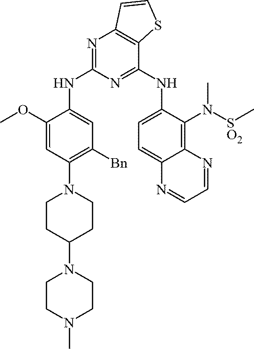 |
| I-70 | 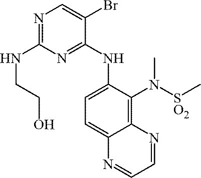 [SMILES: CN(c1c(Nc2nc(NCCO)ncc2Br)ccc2nccnc12)S(C)(=O)=O] | II-1-12 | 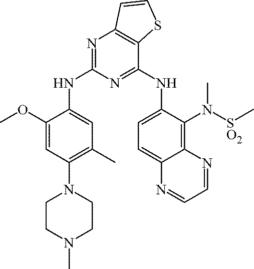 |
| I-71 | 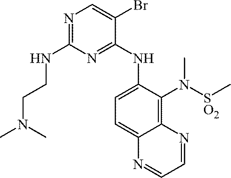 | II-1-13 | 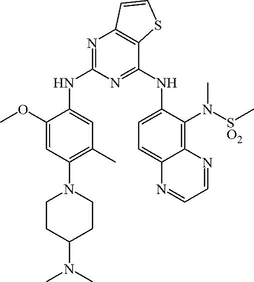 |
| I-72 | 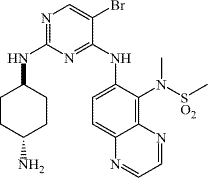 [SMILES: CN(c1c(Nc2nc(N[C@H]3CC[C@H](N)CC3)ncc2Br)ccc2nccnc12)S(C)(=O)=O] | II-1-14 | 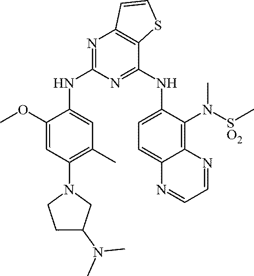 |
| I-73 | 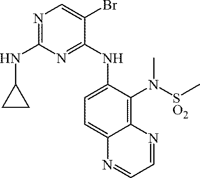 [SMILES: CN(c1c(Nc2nc(NC3CC3)ncc2Br)ccc2nccnc12)S(C)(=O)=O] | II-1-15 | 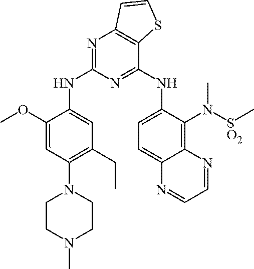 |
| I-74 | 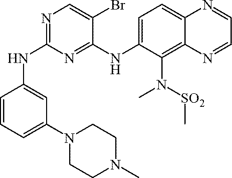 [SMILES: CN1CCN(c2cccc(Nc3ncc(Br)c(Nc4ccc5nccnc5c4N(C)S(C)(=O)=O)n3)c2)CC1] | II-1-16 | 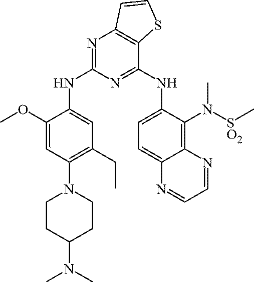 |
| I-75 | 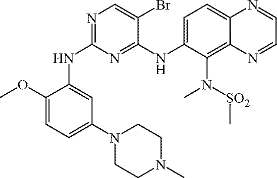 [SMILES: COc1ccc(N2CCN(C)CC2)cc1Nc1ncc(Br)c(Nc2ccc3nccnc3c2N(C)S(C)(=O)=O)n1] | II-1-17 | 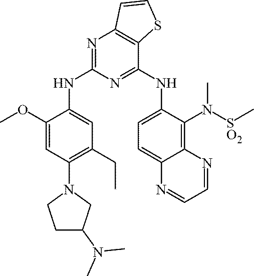 |
| I-76 | 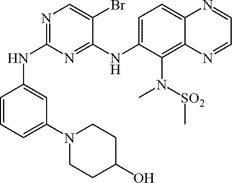 [SMILES: CN(c1c(Nc2nc(Nc3cccc(N4CCC(O)CC4)c3)ncc2Br)ccc2nccnc12)S(C)(=O)=O] | II-1-18 | 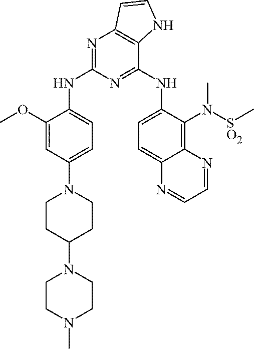 |
| I-77 | 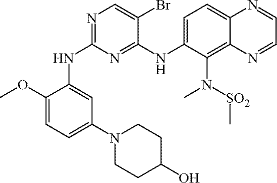 [SMILES: COc1ccc(N2CCC(O)CC2)cc1Nc1ncc(Br)c(Nc2ccc3nccnc3c2N(C)S(C)(=O)=O)n1] | II-1-19 | 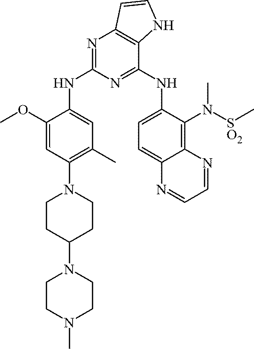 |
| I-78 | 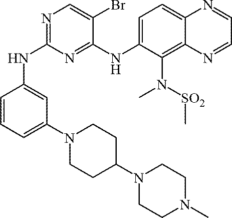 [SMILES: CN1CCN(C2CCN(c3cccc(Nc4ncc(Br)c(Nc5ccc6nccnc6c5N(C)S(C)(=O)=O)n4)c3)CC2)CC1] | II-1-20 | 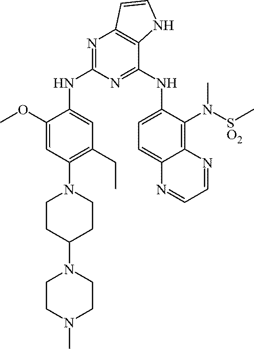 |
| I-79 | 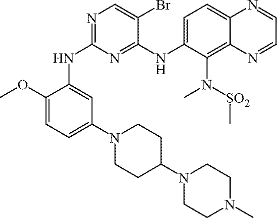 [SMILES: COc1ccc(N2CCC(N3CCN(C)CC3)CC2)cc1Nc1ncc(Br)c(Nc2ccc3nccnc3c2N(C)S(C)(=O)=O)n1] | II-1-21 | 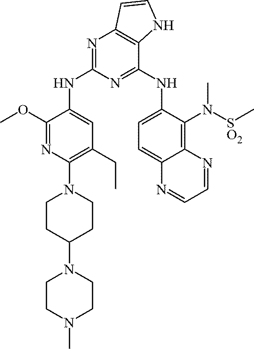 |
| I-80 | 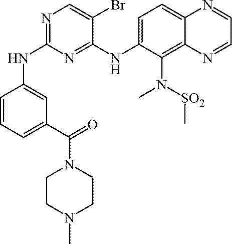 [SMILES: CN1CCN(C(=O)c2cccc(Nc3ncc(Br)c(Nc4ccc5nccnc5c4N(C)S(C)(=O)=O)n3)c2)CC1] | II-1-22 | 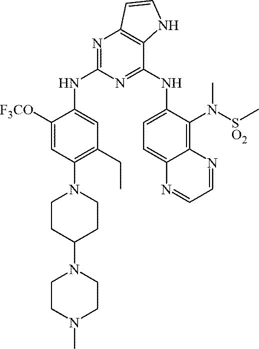 [SMILES: CCc1cc(Nc2nc(Nc3ccc4nccnc4c3N(C)S(C)(=O)=O)c3[nH]ccc3n2)c(OC(F)(F)F)cc1N1CCC(N2CCN(C)CC2)CC1] |
| I-81 | 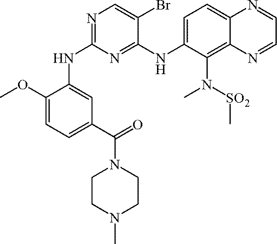 [SMILES: COc1ccc(C(=O)N2CCN(C)CC2)cc1Nc1ncc(Br)c(Nc2ccc3nccnc3c2N(C)S(C)(=O)=O)n1] | II-1-23 | 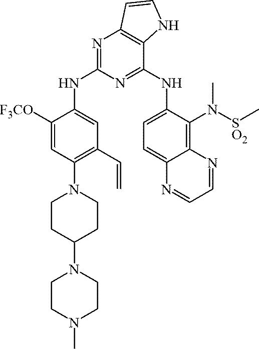 [SMILES: C=Cc1cc(Nc2nc(Nc3ccc4nccnc4c3N(C)S(C)(=O)=O)c3[nH]ccc3n2)c(OC(F)(F)F)cc1N1CCC(N2CCN(C)CC2)CC1] |
| I-82 | 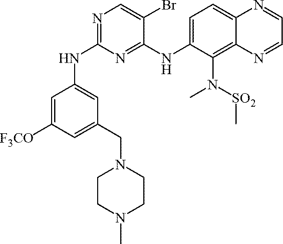 [SMILES: CN1CCN(Cc2cc(Nc3ncc(Br)c(Nc4ccc5nccnc5c4N(C)S(C)(=O)=O)n3)cc(OC(F)(F)F)c2)CC1] | II-1-24 | 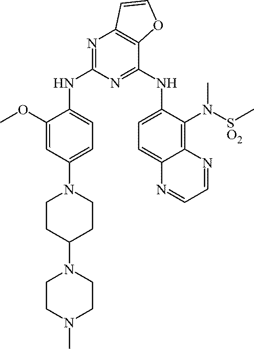 |
| I-83 | 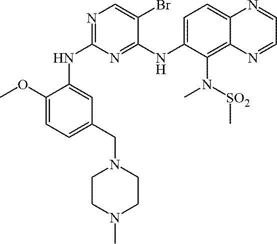 [SMILES: COc1ccc(CN2CCN(C)CC2)cc1Nc1ncc(Br)c(Nc2ccc3nccnc3c2N(C)S(C)(=O)=O)n1] | II-1-25 | 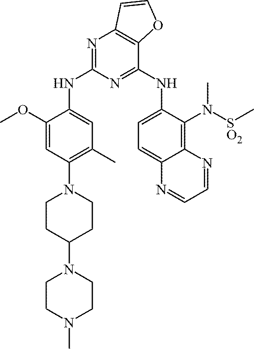 |
| I-84 | 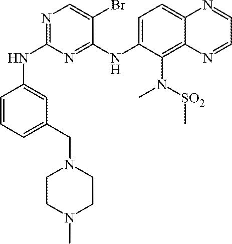 [SMILES: CN1CCN(Cc2cccc(Nc3ncc(Br)c(Nc4ccc5nccnc5c4N(C)S(C)(=O)=O)n3)c2)CC1] | II-1-26 | 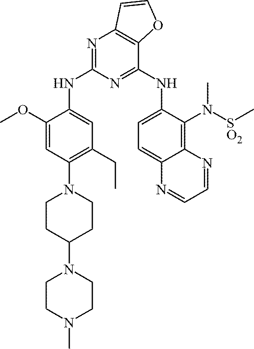 |
| I-85 | 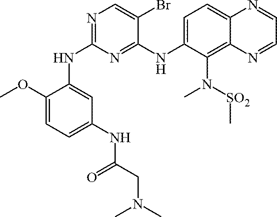 [SMILES: COc1ccc(NC(=O)CN(C)C)cc1Nc1ncc(Br)c(Nc2ccc3nccnc3c2N(C)S(C)(=O)=O)n1] | II-2-1 | 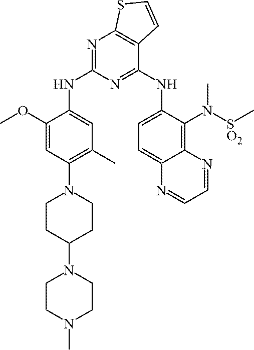 |
| I-86 | 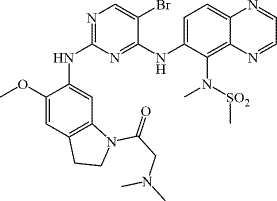 [SMILES: COc1cc2c(cc1Nc1ncc(Br)c(Nc3ccc4nccnc4c3N(C)S(C)(=O)=O)n1)N(C(=O)CN(C)C)CC2] | II-2-2 | 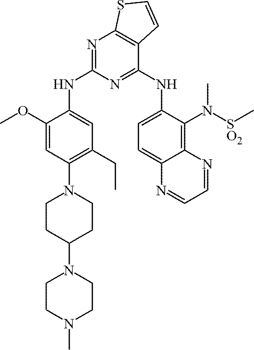 |
| I-87 | 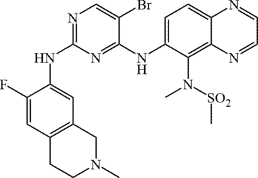 [SMILES: CN1CCc2cc(F)c(Nc3ncc(Br)c(Nc4ccc5nccnc5c4N(C)S(C)(=O)=O)n3)cc2C1] | II-2-3 | 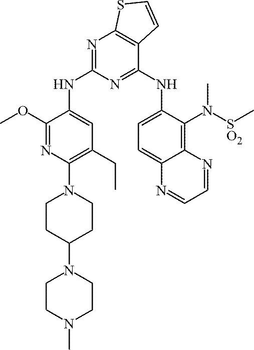 |
| I-88 | 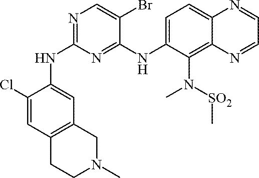 [SMILES: CN1CCc2cc(Cl)c(Nc3ncc(Br)c(Nc4ccc5nccnc5c4N(C)S(C)(=O)=O)n3)cc2C1] | II-2-4 | 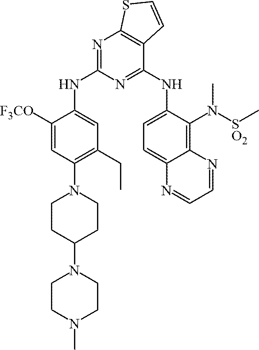 [SMILES: CCc1cc(Nc2nc(Nc3ccc4nccnc4c3N(C)S(C)(=O)=O)c3ccsc3n2)c(OC(F)(F)F)cc1N1CCC(N2CCN(C)CC2)CC1] |
| I-89 | 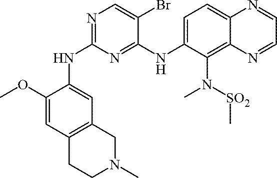 [SMILES: COc1cc2c(cc1Nc1ncc(Br)c(Nc3ccc4nccnc4c3N(C)S(C)(=O)=O)n1)CN(C)CC2] | II-2-5 | 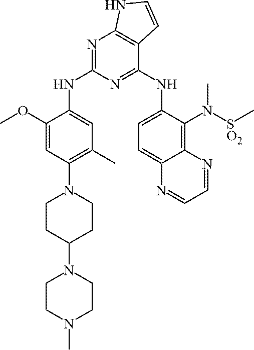 |
| I-90 | 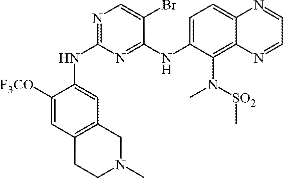 [SMILES: CN1CCc2cc(OC(F)(F)F)c(Nc3ncc(Br)c(Nc4ccc5nccnc5c4N(C)S(C)(=O)=O)n3)cc2C1] | II-2-6 | 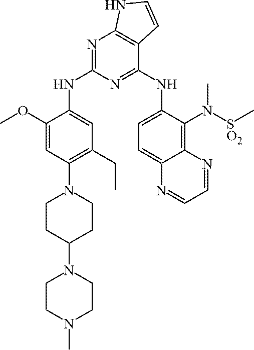 |
| I-91 | 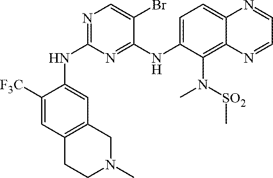 [SMILES: CN1CCc2cc(C(F)(F)F)c(Nc3ncc(Br)c(Nc4ccc5nccnc5c4N(C)S(C)(=O)=O)n3)cc2C1] | II-2-7 | 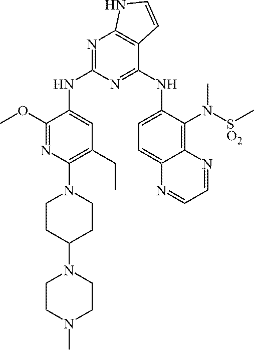 |
| I-92 | 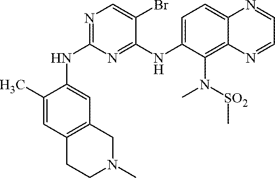 [SMILES: Cc1cc2c(cc1Nc1ncc(Br)c(Nc3ccc4nccnc4c3N(C)S(C)(=O)=O)n1)CN(C)CC2] | II-2-8 | 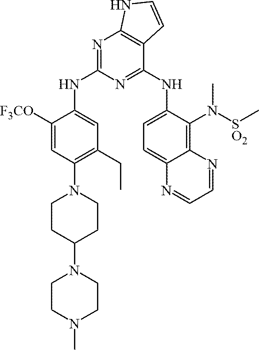 [SMILES: CCc1cc(Nc2nc(Nc3ccc4nccnc4c3N(C)S(C)(=O)=O)c3cc[nH]c3n2)c(OC(F)(F)F)cc1N1CCC(N2CCN(C)CC2)CC1] |
| I-93 | 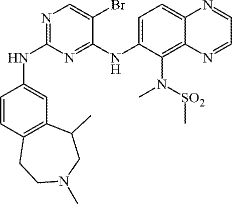 [SMILES: CC1CN(C)CCc2ccc(Nc3ncc(Br)c(Nc4ccc5nccnc5c4N(C)S(C)(=O)=O)n3)cc21] | II-2-9 | 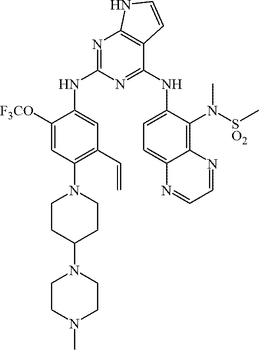 [SMILES: C=Cc1cc(Nc2nc(Nc3ccc4nccnc4c3N(C)S(C)(=O)=O)c3cc[nH]c3n2)c(OC(F)(F)F)cc1N1CCC(N2CCN(C)CC2)CC1] |
| I-94 | 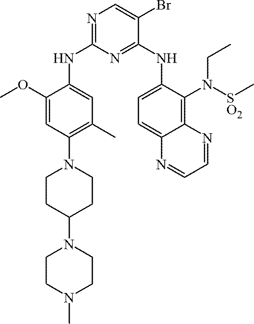 | II-2-10 | 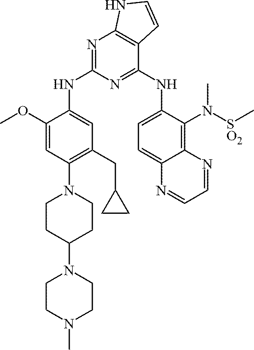 |
| I-95 | 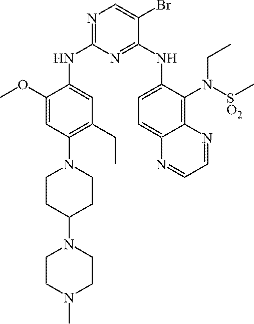 | II-2-11 | 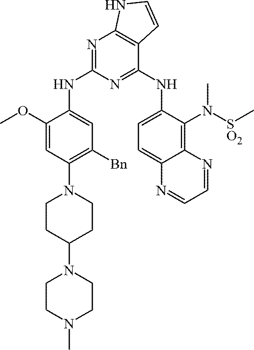 |
| I-96 | 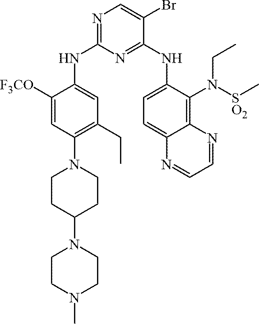 [SMILES: CCc1cc(Nc2ncc(Br)c(Nc3ccc4nccnc4c3N(CC)S(C)(=O)=O)n2)c(OC(F)(F)F)cc1N1CCC(N2CCN(C)CC2)CC1] | II-2-12 | 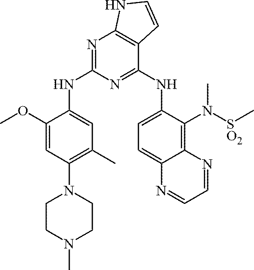 |
| I-97 | 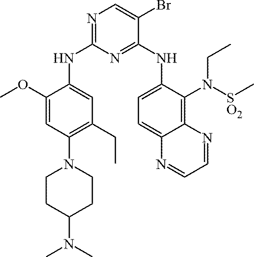 | II-2-13 | 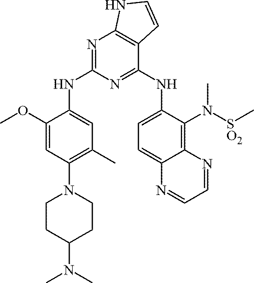 |
| I-98 | 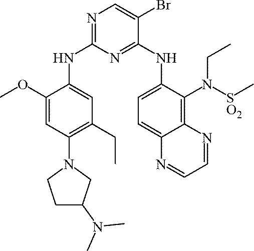 | II-2-14 | 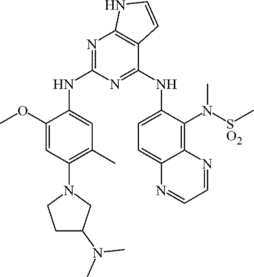 |
| I-99 | 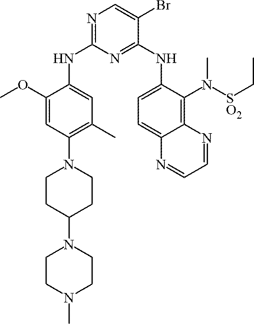 [SMILES: CCS(=O)(=O)N(C)c1c(Nc2nc(Nc3cc(C)c(N4CCC(N5CCN(C)CC5)CC4)cc3OC)ncc2Br)ccc2nccnc12] | II-2-15 | 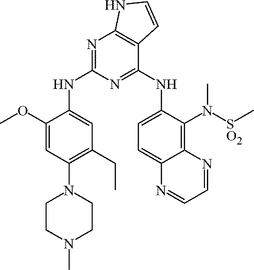 |
| I-100 | 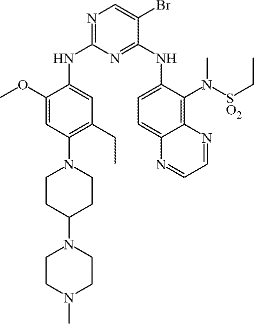 [SMILES: CCc1cc(Nc2ncc(Br)c(Nc3ccc4nccnc4c3N(C)S(=O)(=O)CC)n2)c(OC)cc1N1CCC(N2CCN(C)CC2)CC1] | II-2-16 | 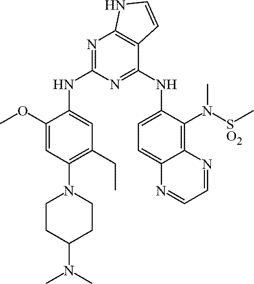 |
| I-101 | 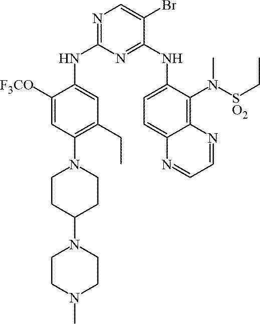 [SMILES: CCc1cc(Nc2ncc(Br)c(Nc3ccc4nccnc4c3N(C)S(=O)(=O)CC)n2)c(OC(F)(F)F)cc1N1CCC(N2CCN(C)CC2)CC1] | II-2-17 | 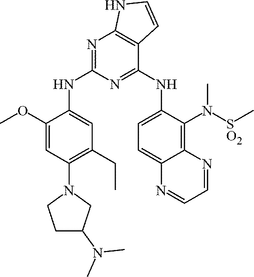 |
| I-102 | 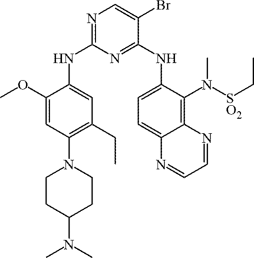 [SMILES: CCc1cc(Nc2ncc(Br)c(Nc3ccc4nccnc4c3N(C)S(=O)(=O)CC)n2)c(OC)cc1N1CCC(N(C)C)CC1] | II-2-18 | 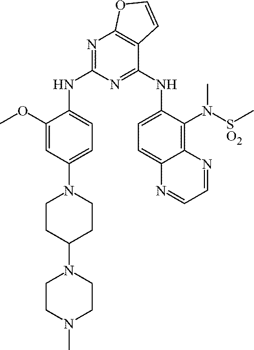 |
| I-103 | 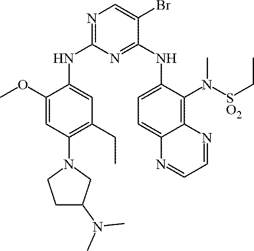 [SMILES: CCc1cc(Nc2ncc(Br)c(Nc3ccc4nccnc4c3N(C)S(=O)(=O)CC)n2)c(OC)cc1N1CCC(N(C)C)C1] | II-2-19 | 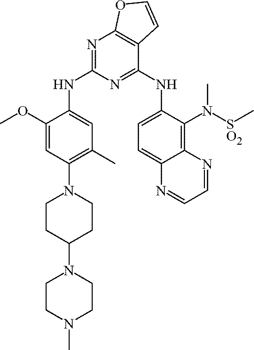 |
| I-104 | 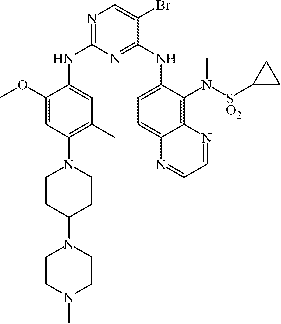 [SMILES: COc1cc(N2CCC(N3CCN(C)CC3)CC2)c(C)cc1Nc1ncc(Br)c(Nc2ccc3nccnc3c2N(C)S(=O)(=O)C2CC2)n1] | II-2-20 | 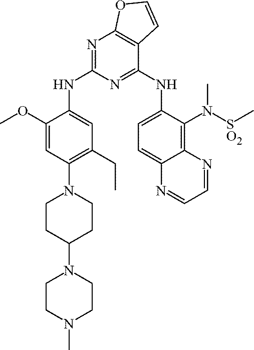 |
| I-105 | 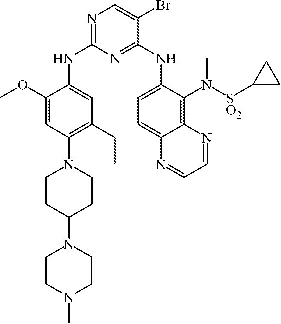 [SMILES: CCc1cc(Nc2ncc(Br)c(Nc3ccc4nccnc4c3N(C)S(=O)(=O)C3CC3)n2)c(OC)cc1N1CCC(N2CCN(C)CC2)CC1] | | |
| I-164 | 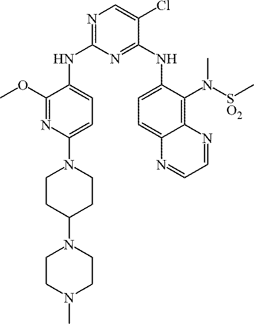 | I-235 | 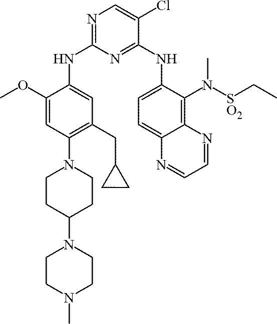 [SMILES: CCS(=O)(=O)N(C)c1c(Nc2nc(Nc3cc(CC4CC4)c(N4CCC(N5CCN(C)CC5)CC4)cc3OC)ncc2Cl)ccc2nccnc12] |
| I-165 | 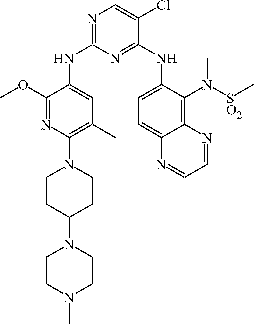 | I-236 | 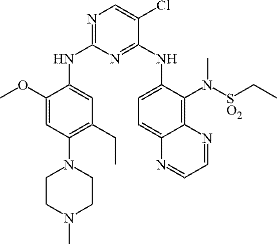 [SMILES: CCc1cc(Nc2ncc(Cl)c(Nc3ccc4nccnc4c3N(C)S(=O)(=O)CC)n2)c(OC)cc1N1CCN(C)CC1] |
| I-166 | 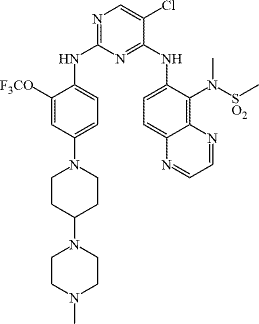 [SMILES: CN1CCN(C2CCN(c3ccc(Nc4ncc(Cl)c(Nc5ccc6nccnc6c5N(C)S(C)(=O)=O)n4)c(OC(F)(F)F)c3)CC2)CC1] | I-237 | 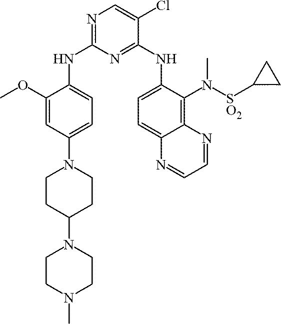 [SMILES: COc1cc(N2CCC(N3CCN(C)CC3)CC2)ccc1Nc1ncc(Cl)c(Nc2ccc3nccnc3c2N(C)S(=O)(=O)C2CC2)n1] |
| I-167 | 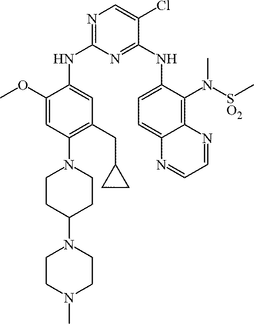 | I-238 | 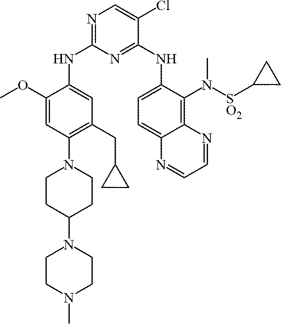 [SMILES: COc1cc(N2CCC(N3CCN(C)CC3)CC2)c(CC2CC2)cc1Nc1ncc(Cl)c(Nc2ccc3nccnc3c2N(C)S(=O)(=O)C2CC2)n1] |
| I-168 | 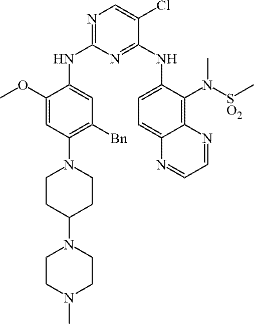 | I-239 | 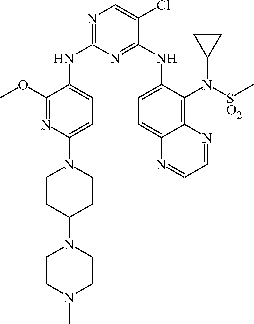 |
| I-169 | 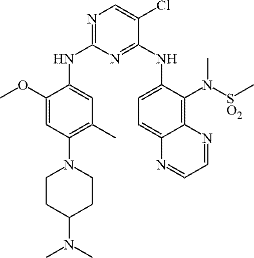 | I-240 | 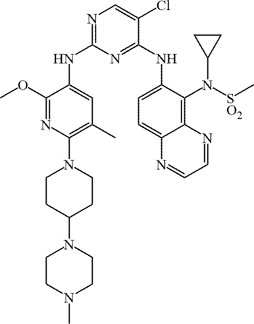 |
| I-170 | 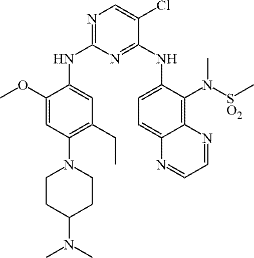 | I-241 | 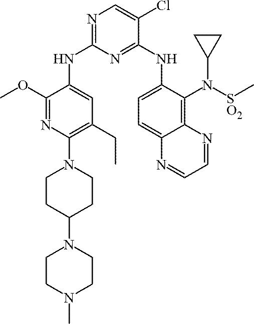 |
| I-171 | 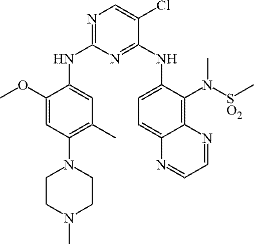 | I-242 | 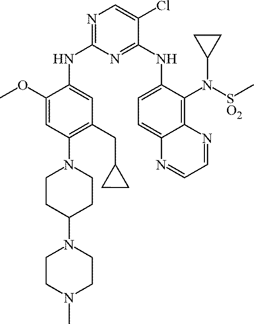 |
| I-172 | 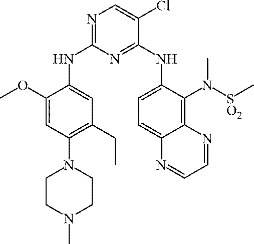 | I-243 | 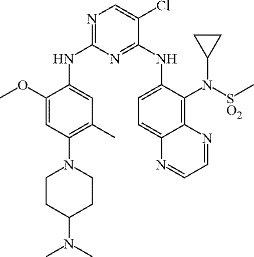 |
| I-173 | 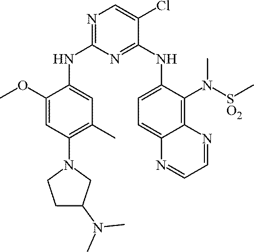 | I-244 | 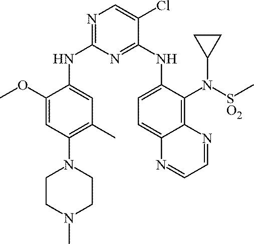 |
| I-174 | 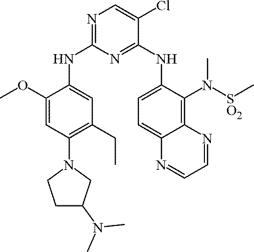 | I-245 | 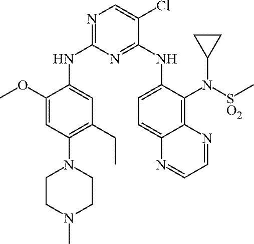 |
| I-175 | 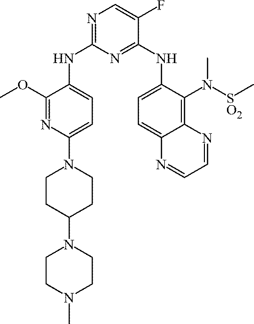 | I-246 | 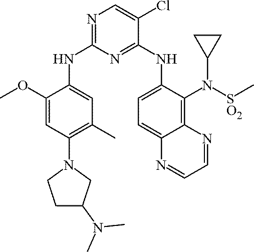 |
| I-176 | 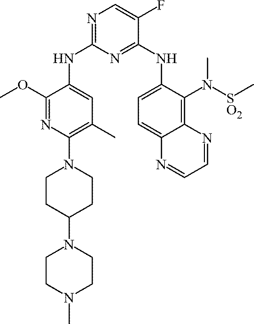 | I-247 | 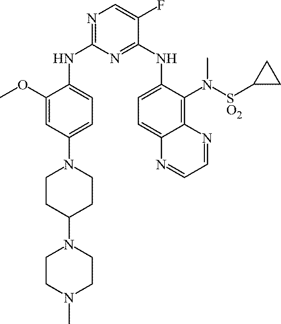 [SMILES: COc1cc(N2CCC(N3CCN(C)CC3)CC2)ccc1Nc1ncc(F)c(Nc2ccc3nccnc3c2N(C)S(=O)(=O)C2CC2)n1] |
| I-177 | 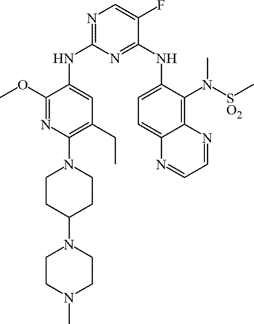 | I-248 | 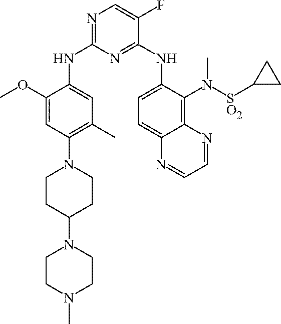 [SMILES: COc1cc(N2CCC(N3CCN(C)CC3)CC2)c(C)cc1Nc1ncc(F)c(Nc2ccc3nccnc3c2N(C)S(=O)(=O)C2CC2)n1] |
| I-178 | 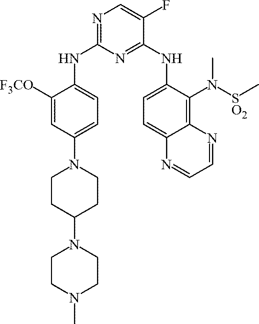 [SMILES: CN1CCN(C2CCN(c3ccc(Nc4ncc(F)c(Nc5ccc6nccnc6c5N(C)S(C)(=O)=O)n4)c(OC(F)(F)F)c3)CC2)CC1] | I-249 | 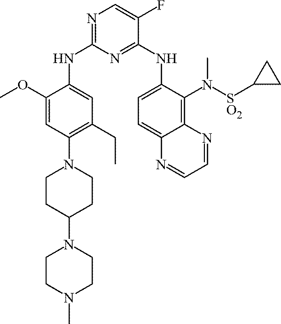 [SMILES: CCc1cc(Nc2ncc(F)c(Nc3ccc4nccnc4c3N(C)S(=O)(=O)C3CC3)n2)c(OC)cc1N1CCC(N2CCN(C)CC2)CC1] |
| I-179 | 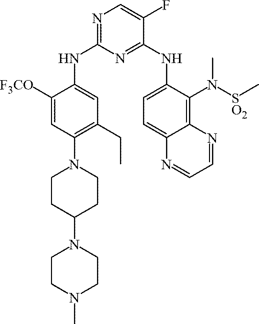 [SMILES: CCc1cc(Nc2ncc(F)c(Nc3ccc4nccnc4c3N(C)S(C)(=O)=O)n2)c(OC(F)(F)F)cc1N1CCC(N2CCN(C)CC2)CC1] | I-250 | 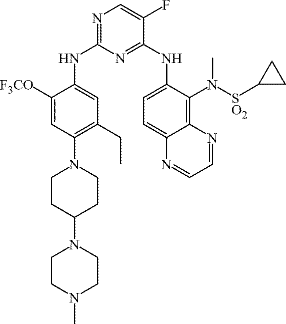 [SMILES: CCc1cc(Nc2ncc(F)c(Nc3ccc4nccnc4c3N(C)S(=O)(=O)C3CC3)n2)c(OC(F)(F)F)cc1N1CCC(N2CCN(C)CC2)CC1] |
| I-180 | 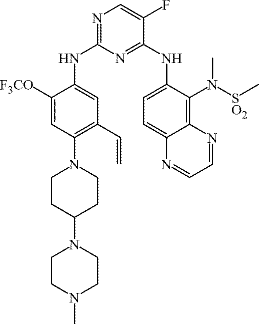 [SMILES: C=Cc1cc(Nc2ncc(F)c(Nc3ccc4nccnc4c3N(C)S(C)(=O)=O)n2)c(OC(F)(F)F)cc1N1CCC(N2CCN(C)CC2)CC1] | I-251 | 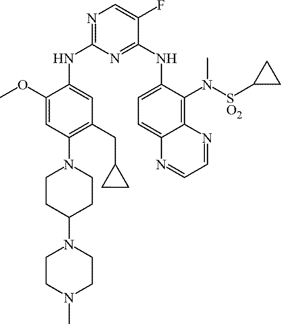 [SMILES: COc1cc(N2CCC(N3CCN(C)CC3)CC2)c(CC2CC2)cc1Nc1ncc(F)c(Nc2ccc3nccnc3c2N(C)S(=O)(=O)C2CC2)n1] |
| I-181 | 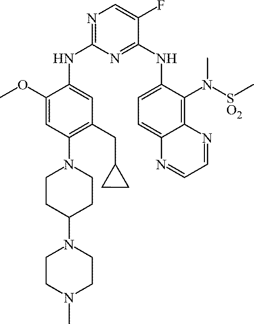 | I-252 | 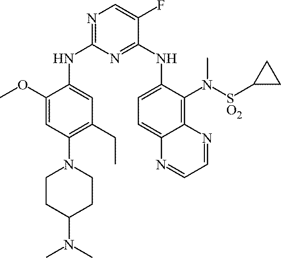 [SMILES: CCc1cc(Nc2ncc(F)c(Nc3ccc4nccnc4c3N(C)S(=O)(=O)C3CC3)n2)c(OC)cc1N1CCC(N(C)C)CC1] |
| I-182 | 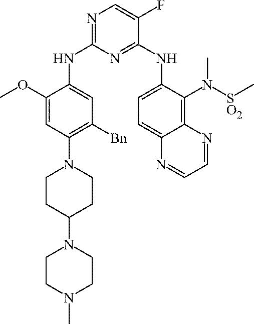 | I-253 | 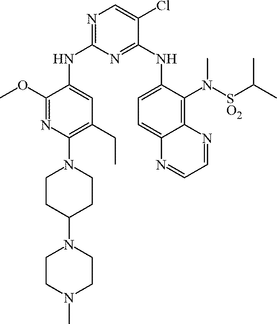 [SMILES: CCc1cc(Nc2ncc(Cl)c(Nc3ccc4nccnc4c3N(C)S(=O)(=O)C(C)C)n2)c(OC)nc1N1CCC(N2CCN(C)CC2)CC1] |
| I-183 | 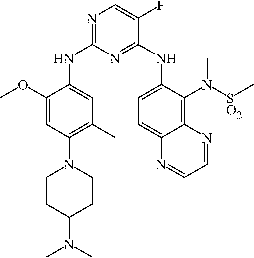 | I-254 | 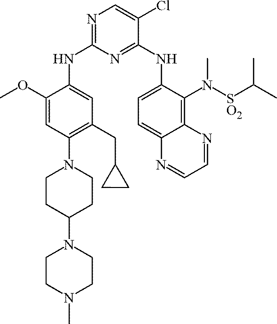 [SMILES: COc1cc(N2CCC(N3CCN(C)CC3)CC2)c(CC2CC2)cc1Nc1ncc(Cl)c(Nc2ccc3nccnc3c2N(C)S(=O)(=O)C(C)C)n1] |
| I-184 | 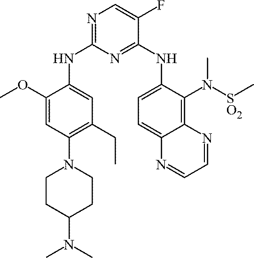 | I-255 | 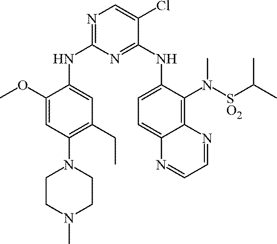 [SMILES: CCc1cc(Nc2ncc(Cl)c(Nc3ccc4nccnc4c3N(C)S(=O)(=O)C(C)C)n2)c(OC)cc1N1CCN(C)CC1] |
| I-185 | 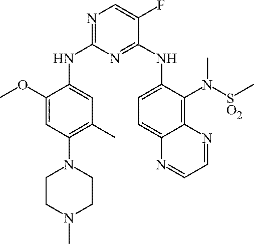 | I-256 | 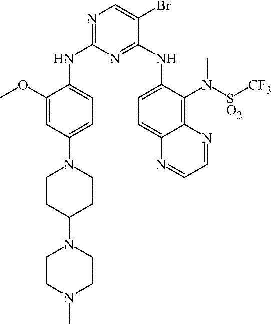 [SMILES: COc1cc(N2CCC(N3CCN(C)CC3)CC2)ccc1Nc1ncc(Br)c(Nc2ccc3nccnc3c2N(C)S(=O)(=O)C(F)(F)F)n1] |
| I-186 | 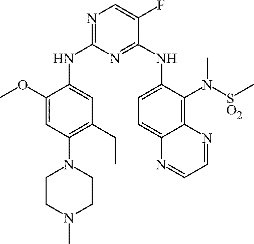 | I-257 | 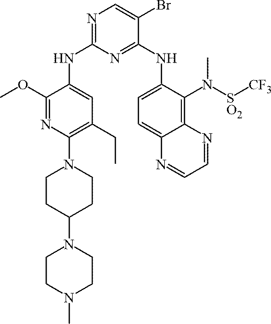 [SMILES: CCc1cc(Nc2ncc(Br)c(Nc3ccc4nccnc4c3N(C)S(=O)(=O)C(F)(F)F)n2)c(OC)nc1N1CCC(N2CCN(C)CC2)CC1] |
| I-187 | 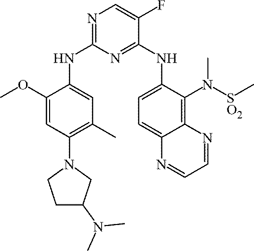 | I-258 | 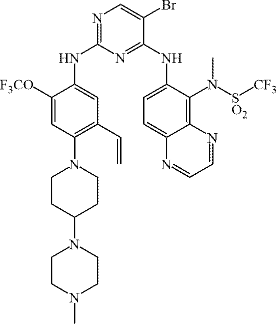 [SMILES: C=Cc1cc(Nc2ncc(Br)c(Nc3ccc4nccnc4c3N(C)S(=O)(=O)C(F)(F)F)n2)c(OC(F)(F)F)cc1N1CCC(N2CCN(C)CC2)CC1] |
| I-188 | 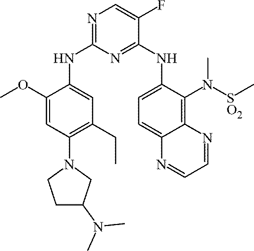 | I-259 | 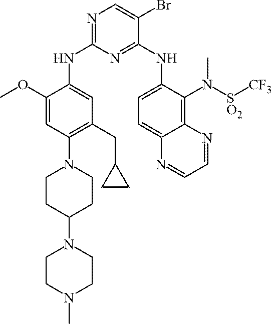 [SMILES: COc1cc(N2CCC(N3CCN(C)CC3)CC2)c(CC2CC2)cc1Nc1ncc(Br)c(Nc2ccc3nccnc3c2N(C)S(=O)(=O)C(F)(F)F)n1] |
| I-189 | 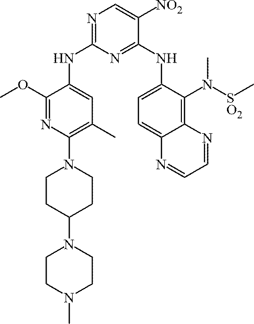 | I-260 | 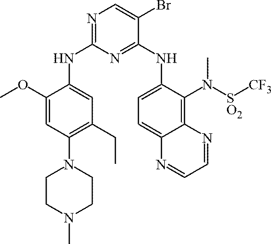 [SMILES: CCc1cc(Nc2ncc(Br)c(Nc3ccc4nccnc4c3N(C)S(=O)(=O)C(F)(F)F)n2)c(OC)cc1N1CCN(C)CC1] |
| I-190 | 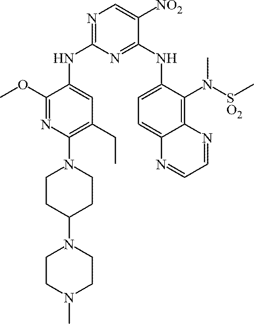 | I-261 | 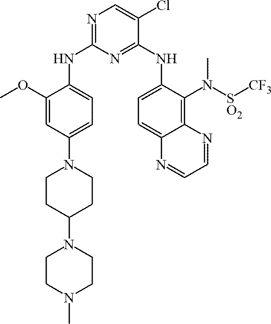 [SMILES: COc1cc(N2CCC(N3CCN(C)CC3)CC2)ccc1Nc1ncc(Cl)c(Nc2ccc3nccnc3c2N(C)S(=O)(=O)C(F)(F)F)n1] |
| I-191 | 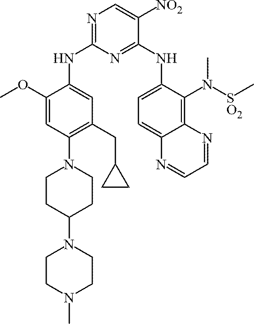 | I-262 | 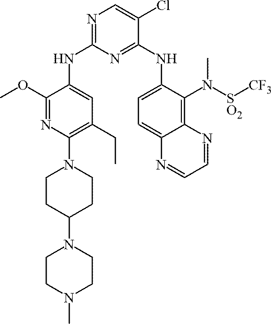 [SMILES: CCc1cc(Nc2ncc(Cl)c(Nc3ccc4nccnc4c3N(C)S(=O)(=O)C(F)(F)F)n2)c(OC)nc1N1CCC(N2CCN(C)CC2)CC1] |
| I-192 | 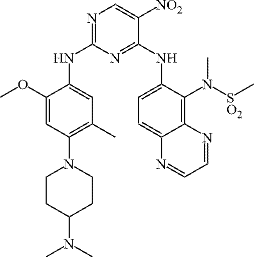 | I-263 | 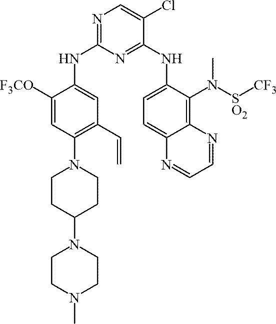 [SMILES: C=Cc1cc(Nc2ncc(Cl)c(Nc3ccc4nccnc4c3N(C)S(=O)(=O)C(F)(F)F)n2)c(OC(F)(F)F)cc1N1CCC(N2CCN(C)CC2)CC1] |
| I-193 | 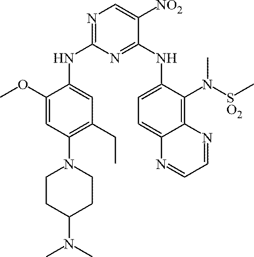 | I-264 | 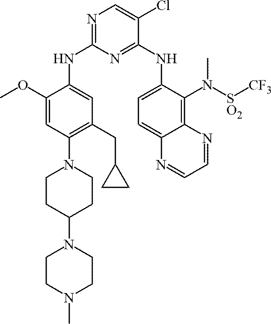 [SMILES: COc1cc(N2CCC(N3CCN(C)CC3)CC2)c(CC2CC2)cc1Nc1ncc(Cl)c(Nc2ccc3nccnc3c2N(C)S(=O)(=O)C(F)(F)F)n1] |
| I-194 | 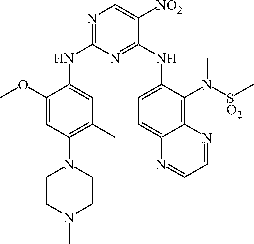 | I-265 | 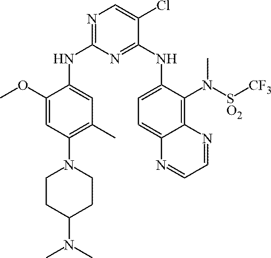 [SMILES: COc1cc(N2CCC(N(C)C)CC2)c(C)cc1Nc1ncc(Cl)c(Nc2ccc3nccnc3c2N(C)S(=O)(=O)C(F)(F)F)n1] |
| I-195 | 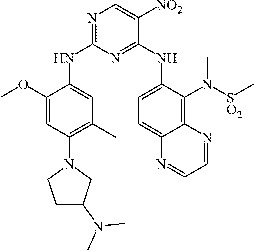 | I-266 | 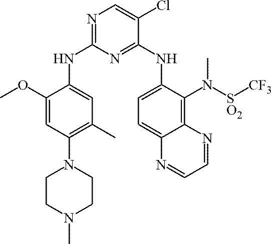 [SMILES: COc1cc(N2CCN(C)CC2)c(C)cc1Nc1ncc(Cl)c(Nc2ccc3nccnc3c2N(C)S(=O)(=O)C(F)(F)F)n1] |
| I-196 | 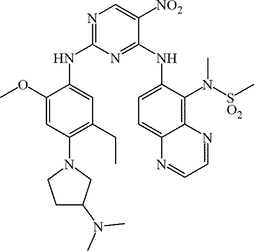 | I-267 | 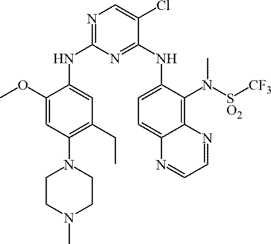 [SMILES: CCc1cc(Nc2ncc(Cl)c(Nc3ccc4nccnc4c3N(C)S(=O)(=O)C(F)(F)F)n2)c(OC)cc1N1CCN(C)CC1] |
| I-197 | 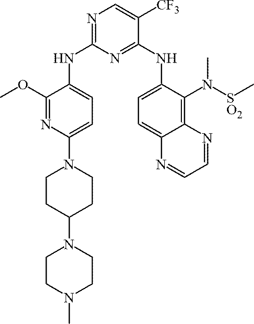 | I-268 | 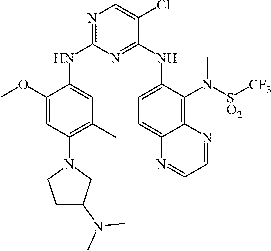 [SMILES: COc1cc(N2CCC(N(C)C)C2)c(C)cc1Nc1ncc(Cl)c(Nc2ccc3nccnc3c2N(C)S(=O)(=O)C(F)(F)F)n1] |
| I-198 | 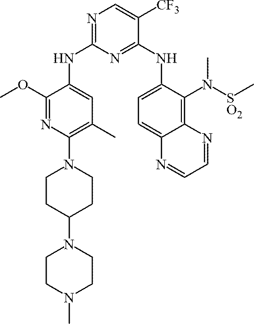 | I-269 | 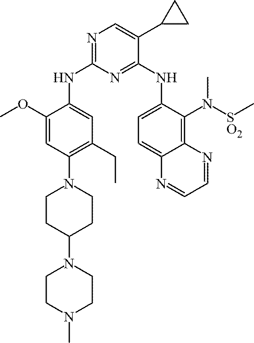 |
| I-199 | 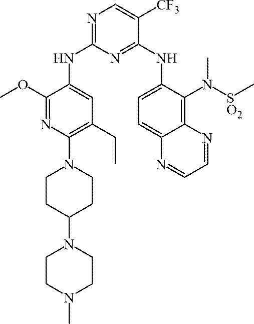 | I-270 | 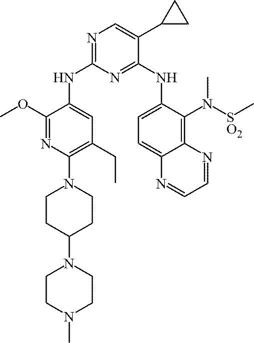 |
| I-200 | 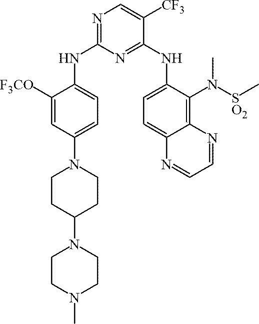 [SMILES: CN1CCN(C2CCN(c3ccc(Nc4ncc(C(F)(F)F)c(Nc5ccc6nccnc6c5N(C)S(C)(=O)=O)n4)c(OC(F)(F)F)c3)CC2)CC1] | I-271 | 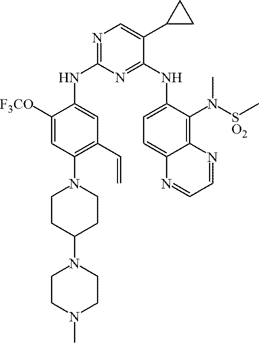 [SMILES: C=Cc1cc(Nc2ncc(C3CC3)c(Nc3ccc4nccnc4c3N(C)S(C)(=O)=O)n2)c(OC(F)(F)F)cc1N1CCC(N2CCN(C)CC2)CC1] |
| I-201 | 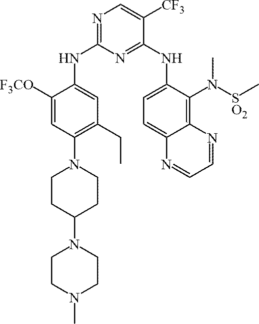 [SMILES: CCc1cc(Nc2ncc(C(F)(F)F)c(Nc3ccc4nccnc4c3N(C)S(C)(=O)=O)n2)c(OC(F)(F)F)cc1N1CCC(N2CCN(C)CC2)CC1] | I-272 | 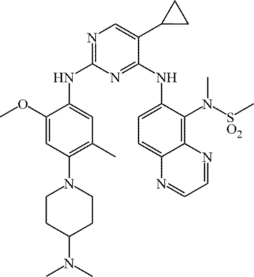 |
| I-202 | 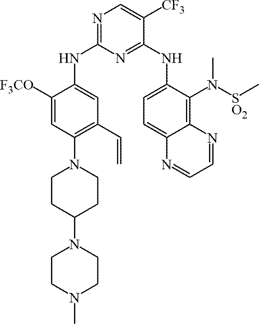 [SMILES: C=Cc1cc(Nc2ncc(C(F)(F)F)c(Nc3ccc4nccnc4c3N(C)S(C)(=O)=O)n2)c(OC(F)(F)F)cc1N1CCC(N2CCN(C)CC2)CC1] | I-273 | 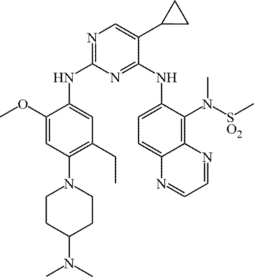 |
| I-203 | 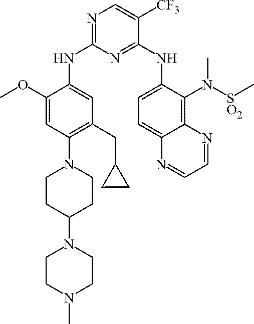 | I-274 | 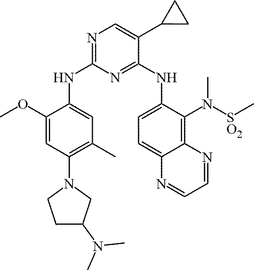 |
| I-204 | 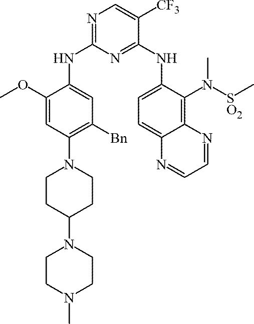 | I-275 | 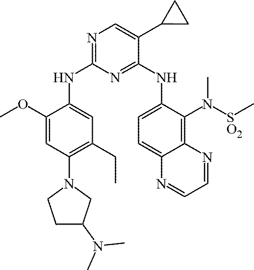 |
| I-205 | 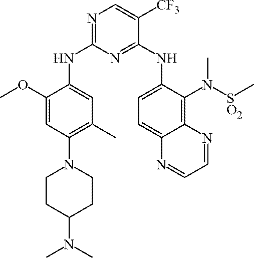 | I-276 | 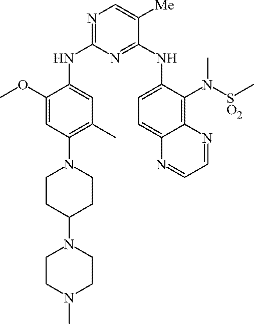 |
| I-206 | 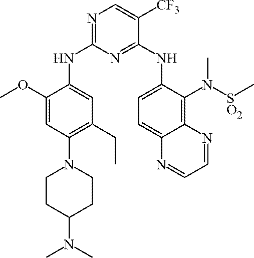 | I-277 | 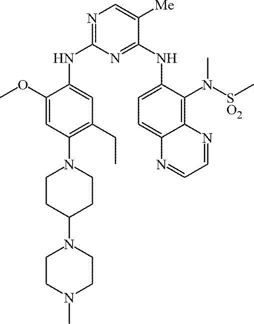 |
| I-207 | 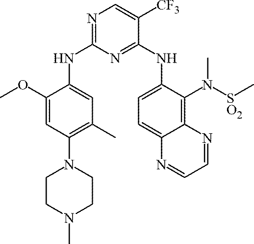 | I-278 | 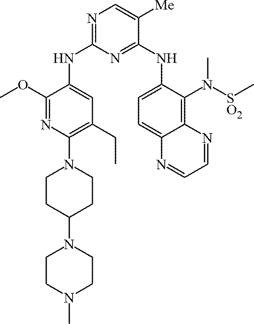 |
| I-208 | 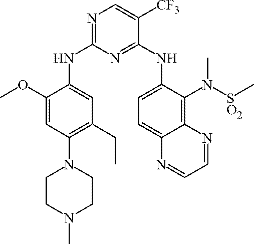 | I-279 | 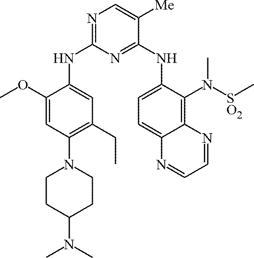 |
| I-209 | 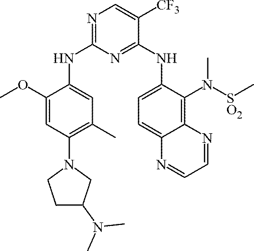 | I-280 | 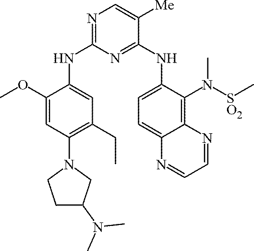 |
| I-210 | 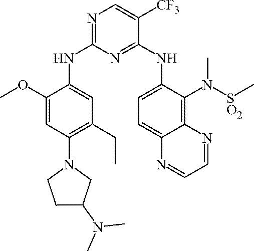 | I-281 | 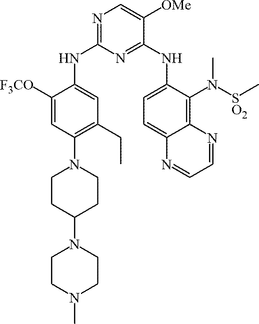 [SMILES: CCc1cc(Nc2ncc(OC)c(Nc3ccc4nccnc4c3N(C)S(C)(=O)=O)n2)c(OC(F)(F)F)cc1N1CCC(N2CCN(C)CC2)CC1] |
| I-211 | 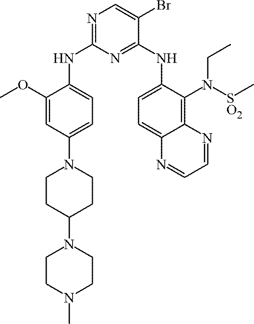 | I-282 | 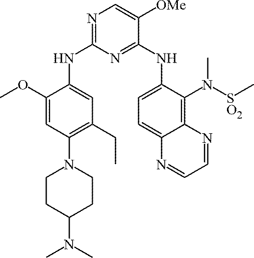 |
| I-212 | 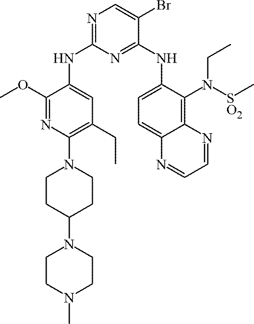 | I-283 | 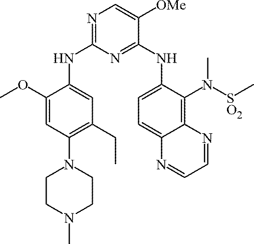 |
| I-213 | 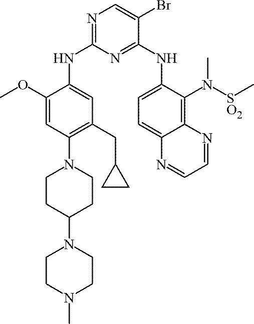 | II-1-27 | 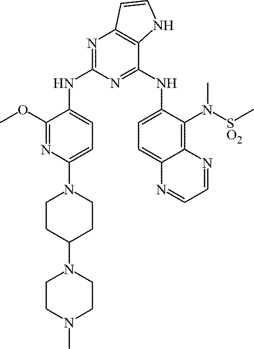 |
| I-214 | 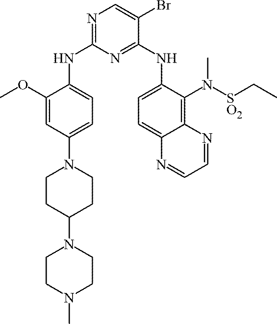 [SMILES: CCS(=O)(=O)N(C)c1c(Nc2nc(Nc3ccc(N4CCC(N5CCN(C)CC5)CC4)cc3OC)ncc2Br)ccc2nccnc12] | II-1-28 | 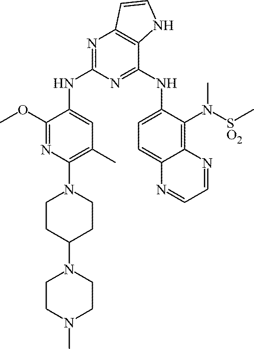 |
| I-215 | 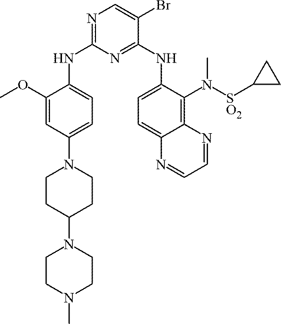 [SMILES: COc1cc(N2CCC(N3CCN(C)CC3)CC2)ccc1Nc1ncc(Br)c(Nc2ccc3nccnc3c2N(C)S(=O)(=O)C2CC2)n1] | II-1-29 | 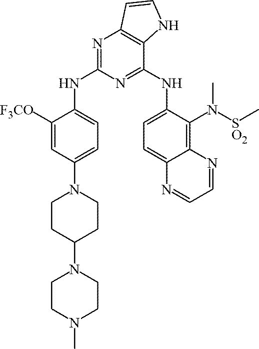 [SMILES: CN1CCN(C2CCN(c3ccc(Nc4nc(Nc5ccc6nccnc6c5N(C)S(C)(=O)=O)c5[nH]ccc5n4)c(OC(F)(F)F)c3)CC2)CC1] |
| I-216 | 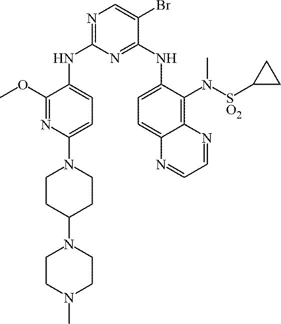 [SMILES: COc1nc(N2CCC(N3CCN(C)CC3)CC2)ccc1Nc1ncc(Br)c(Nc2ccc3nccnc3c2N(C)S(=O)(=O)C2CC2)n1] | II-1-30 | 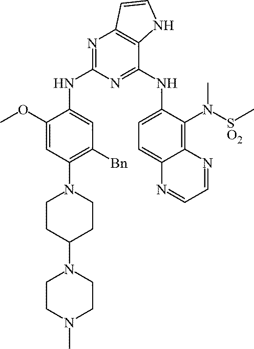 |
| I-217 | 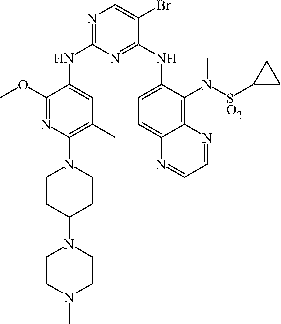 [SMILES: COc1nc(N2CCC(N3CCN(C)CC3)CC2)c(C)cc1Nc1ncc(Br)c(Nc2ccc3nccnc3c2N(C)S(=O)(=O)C2CC2)n1] | II-1-31 | 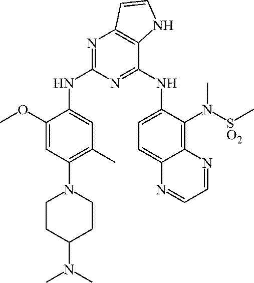 |
| I-218 | 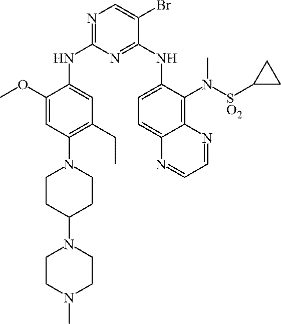 [SMILES: CCc1cc(Nc2ncc(Br)c(Nc3ccc4nccnc4c3N(C)S(=O)(=O)C3CC3)n2)c(OC)cc1N1CCC(N2CCN(C)CC2)CC1] | II-1-32 | 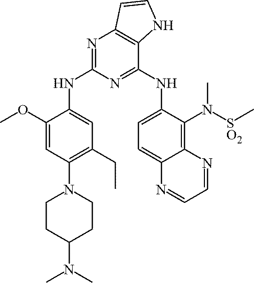 |
| I-219 | 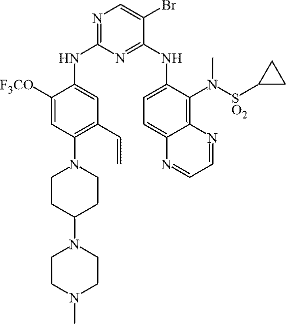 [SMILES: C=Cc1cc(Nc2ncc(Br)c(Nc3ccc4nccnc4c3N(C)S(=O)(=O)C3CC3)n2)c(OC(F)(F)F)cc1N1CCC(N2CCN(C)CC2)CC1] | II-1-33 | 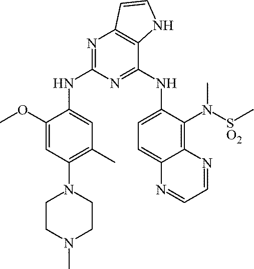 |
| I-220 | 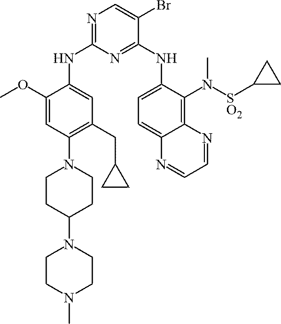 [SMILES: COc1cc(N2CCC(N3CCN(C)CC3)CC2)c(CC2CC2)cc1Nc1ncc(Br)c(Nc2ccc3nccnc3c2N(C)S(=O)(=O)C2CC2)n1] | II-1-34 | 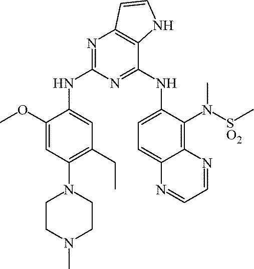 |
| I-221 | 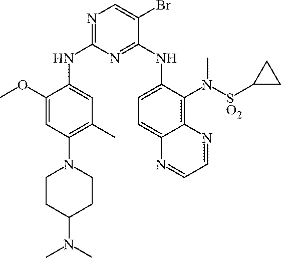 [SMILES: COc1cc(N2CCC(N(C)C)CC2)c(C)cc1Nc1ncc(Br)c(Nc2ccc3nccnc3c2N(C)S(=O)(=O)C2CC2)n1] | II-1-35 | 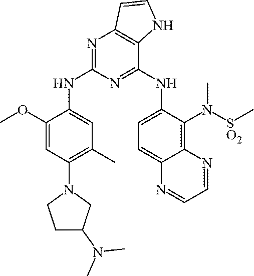 |
| I-222 | 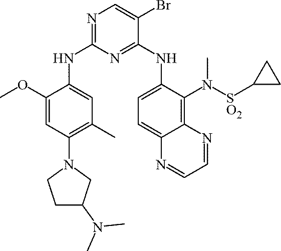 [SMILES: COc1cc(N2CCC(N(C)C)C2)c(C)cc1Nc1ncc(Br)c(Nc2ccc3nccnc3c2N(C)S(=O)(=O)C2CC2)n1] | II-1-36 | 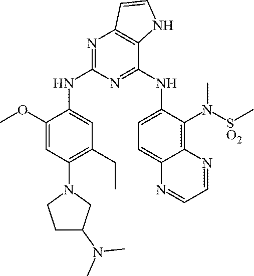 |
| I-223 | 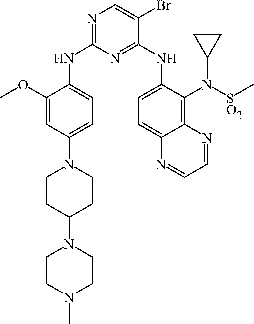 | II-1-37 | 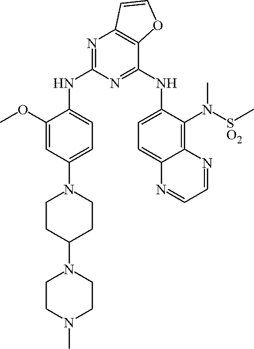 |
| I-224 | 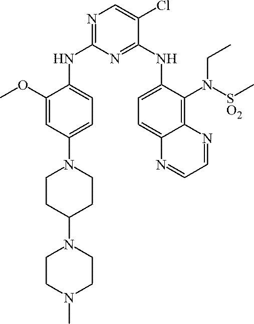 | II-1-38 | 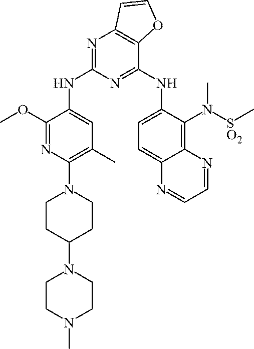 |
| I-225 | 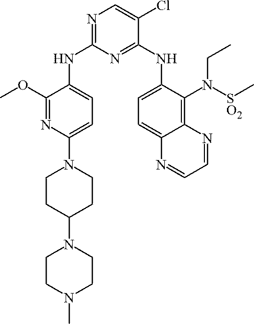 | II-1-39 | 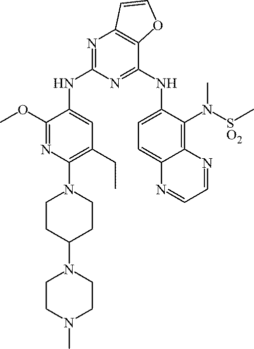 |
| I-226 | 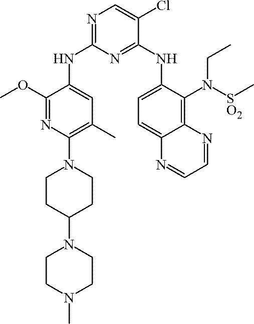 | II-1-40 | 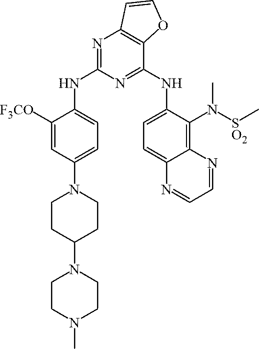 [SMILES: CN1CCN(C2CCN(c3ccc(Nc4nc(Nc5ccc6nccnc6c5N(C)S(C)(=O)=O)c5occc5n4)c(OC(F)(F)F)c3)CC2)CC1] |
| I-227 | 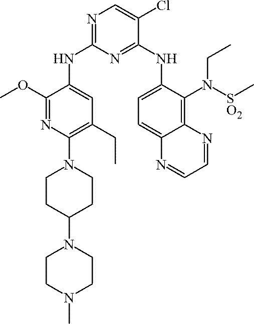 | II-1-41 | 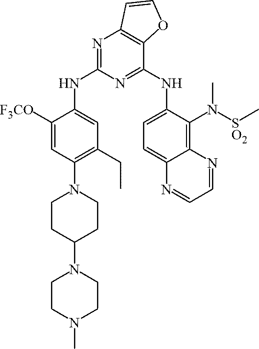 [SMILES: CCc1cc(Nc2nc(Nc3ccc4nccnc4c3N(C)S(C)(=O)=O)c3occc3n2)c(OC(F)(F)F)cc1N1CCC(N2CCN(C)CC2)CC1] |
| I-228 | 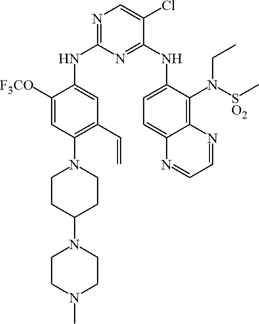 [SMILES: C=Cc1cc(Nc2ncc(Cl)c(Nc3ccc4nccnc4c3N(CC)S(C)(=O)=O)n2)c(OC(F)(F)F)cc1N1CCC(N2CCN(C)CC2)CC1] | II-1-42 | 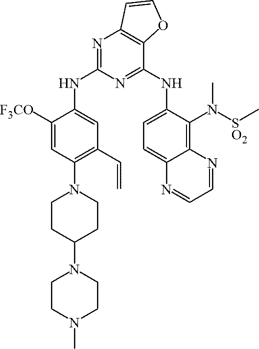 [SMILES: C=Cc1cc(Nc2nc(Nc3ccc4nccnc4c3N(C)S(C)(=O)=O)c3occc3n2)c(OC(F)(F)F)cc1N1CCC(N2CCN(C)CC2)CC1] |
| I-229 | 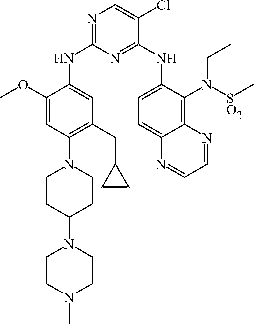 | II-1-43 | 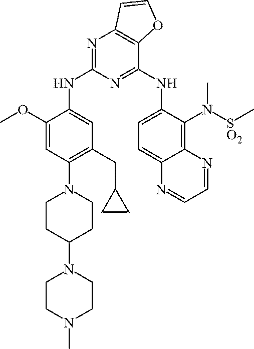 |
| I-230 | 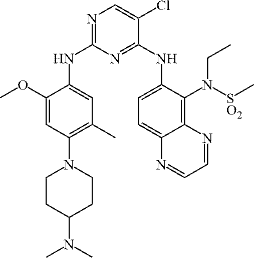 | II-1-44 | 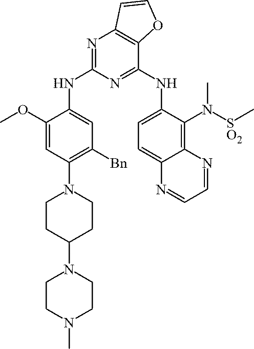 |
| I-231 | 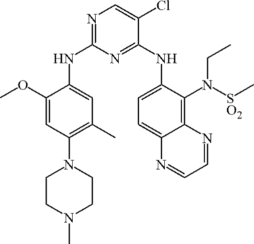 | II-2-21 | 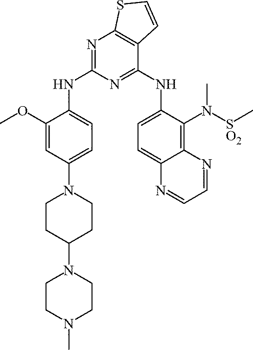 |
| I-232 | 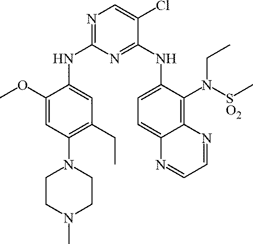 | II-2-22 | 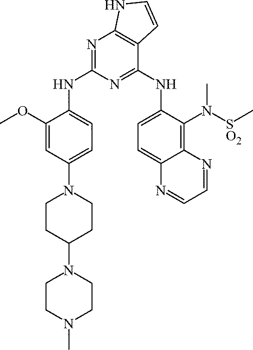 |
| I-233 | 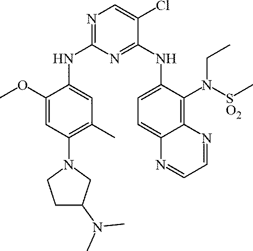 | | |
| I-234 | 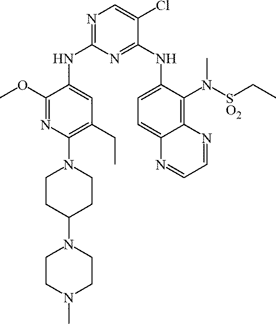 [SMILES: CCc1cc(Nc2ncc(Cl)c(Nc3ccc4nccnc4c3N(C)S(=O)(=O)CC)n2)c(OC)nc1N1CCC(N2CCN(C)CC2)CC1] | | |
| I-284 | 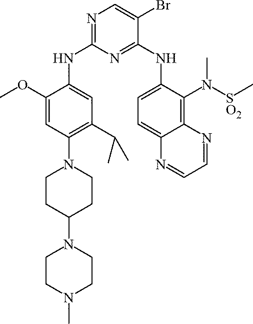 | I-352 | 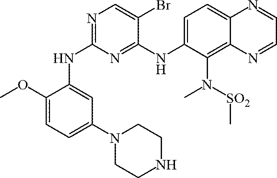 [SMILES: COc1ccc(N2CCNCC2)cc1Nc1ncc(Br)c(Nc2ccc3nccnc3c2N(C)S(C)(=O)=O)n1] |
| I-285 | 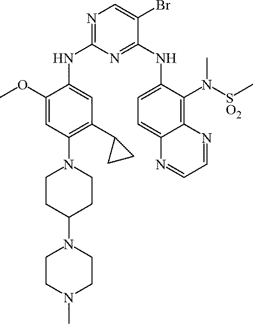 | I-353 | 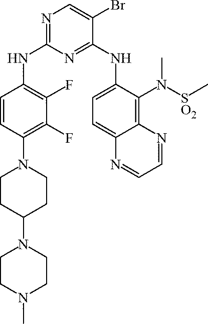 [SMILES: CN1CCN(C2CCN(c3ccc(Nc4ncc(Br)c(Nc5ccc6nccnc6c5N(C)S(C)(=O)=O)n4)c(F)c3F)CC2)CC1] |
| I-286 | 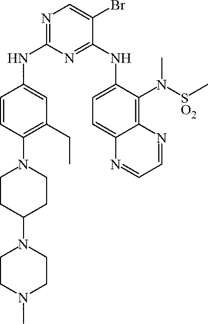 [SMILES: CCc1cc(Nc2ncc(Br)c(Nc3ccc4nccnc4c3N(C)S(C)(=O)=O)n2)ccc1N1CCC(N2CCN(C)CC2)CC1] | I-354 | 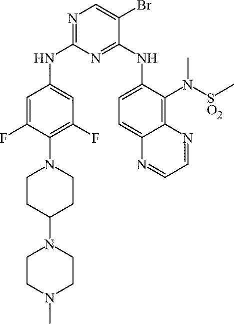 [SMILES: CN1CCN(C2CCN(c3c(F)cc(Nc4ncc(Br)c(Nc5ccc6nccnc6c5N(C)S(C)(=O)=O)n4)cc3F)CC2)CC1] |
| I-287 | 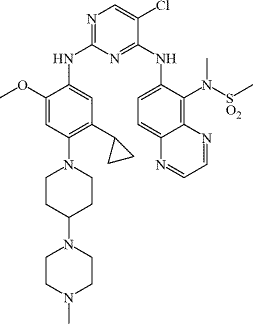 | I-355 | 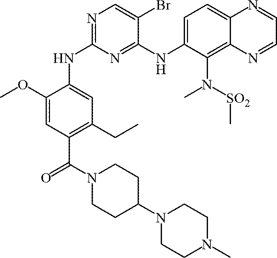 [SMILES: CCc1cc(Nc2ncc(Br)c(Nc3ccc4nccnc4c3N(C)S(C)(=O)=O)n2)c(OC)cc1C(=O)N1CCC(N2CCN(C)CC2)CC1] |
| I-288 | 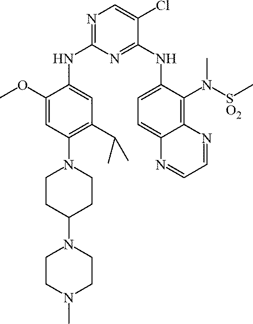 | I-356 | 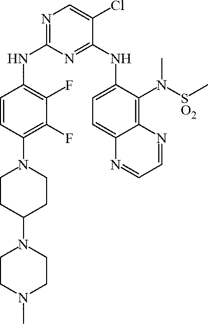 [SMILES: CN1CCN(C2CCN(c3ccc(Nc4ncc(Cl)c(Nc5ccc6nccnc6c5N(C)S(C)(=O)=O)n4)c(F)c3F)CC2)CC1] |
| I-289 | 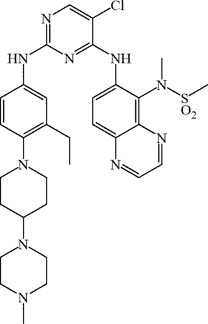 [SMILES: CCc1cc(Nc2ncc(Cl)c(Nc3ccc4nccnc4c3N(C)S(C)(=O)=O)n2)ccc1N1CCC(N2CCN(C)CC2)CC1] | I-357 | 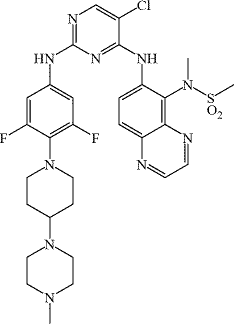 [SMILES: CN1CCN(C2CCN(c3c(F)cc(Nc4ncc(Cl)c(Nc5ccc6nccnc6c5N(C)S(C)(=O)=O)n4)cc3F)CC2)CC1] |
| I-290 | 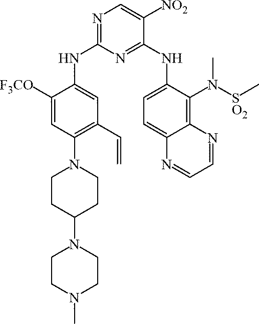 [SMILES: C=Cc1cc(Nc2ncc([N+](=O)[O-])c(Nc3ccc4nccnc4c3N(C)S(C)(=O)=O)n2)c(OC(F)(F)F)cc1N1CCC(N2CCN(C)CC2)CC1] | I-358 | 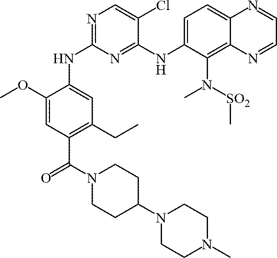 [SMILES: CCc1cc(Nc2ncc(Cl)c(Nc3ccc4nccnc4c3N(C)S(C)(=O)=O)n2)c(OC)cc1C(=O)N1CCC(N2CCN(C)CC2)CC1] |
| I-291 | 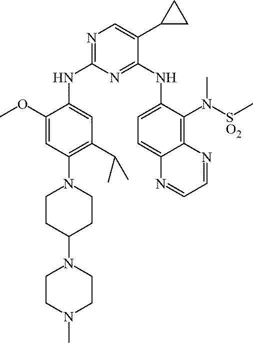 | I-359 | 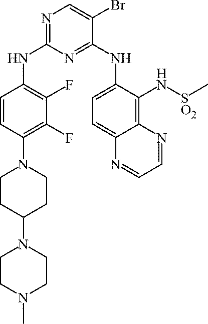 [SMILES: CN1CCN(C2CCN(c3ccc(Nc4ncc(Br)c(Nc5ccc6nccnc6c5NS(C)(=O)=O)n4)c(F)c3F)CC2)CC1] |
| I-292 | 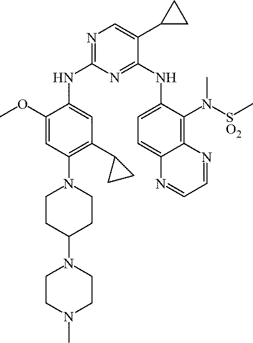 | I-360 | 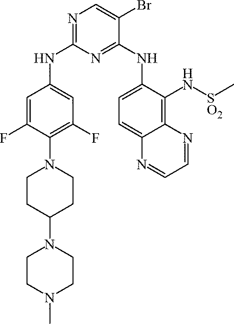 [SMILES: CN1CCN(C2CCN(c3c(F)cc(Nc4ncc(Br)c(Nc5ccc6nccnc6c5NS(C)(=O)=O)n4)cc3F)CC2)CC1] |
| I-293 | 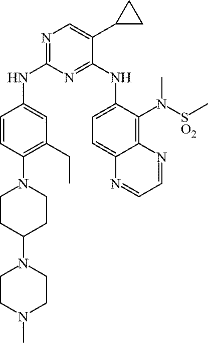 [SMILES: CCc1cc(Nc2ncc(C3CC3)c(Nc3ccc4nccnc4c3N(C)S(C)(=O)=O)n2)ccc1N1CCC(N2CCN(C)CC2)CC1] | I-361 | 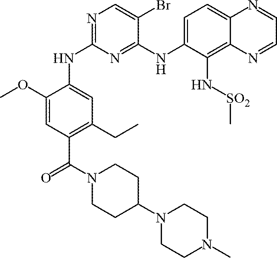 [SMILES: CCc1cc(Nc2ncc(Br)c(Nc3ccc4nccnc4c3NS(C)(=O)=O)n2)c(OC)cc1C(=O)N1CCC(N2CCN(C)CC2)CC1] |
| I-294 | 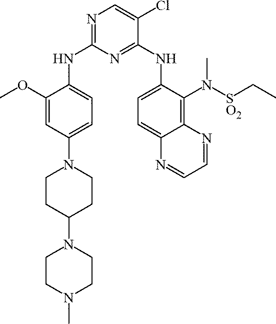 [SMILES: CCS(=O)(=O)N(C)c1c(Nc2nc(Nc3ccc(N4CCC(N5CCN(C)CC5)CC4)cc3OC)ncc2Cl)ccc2nccnc12] | I-362 | 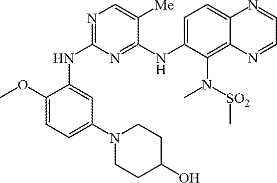 [SMILES: COc1ccc(N2CCC(O)CC2)cc1Nc1ncc(C)c(Nc2ccc3nccnc3c2N(C)S(C)(=O)=O)n1] |
| I-295 | 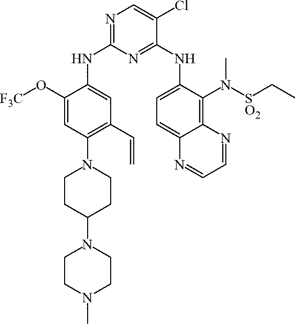 [SMILES: C=Cc1cc(Nc2ncc(Cl)c(Nc3ccc4nccnc4c3N(C)S(=O)(=O)CC)n2)c(OC(F)(F)F)cc1N1CCC(N2CCN(C)CC2)CC1] | I-363 | 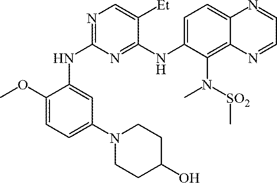 [SMILES: CCc1cnc(Nc2cc(N3CCC(O)CC3)ccc2OC)nc1Nc1ccc2nccnc2c1N(C)S(C)(=O)=O] |
| I-296 | 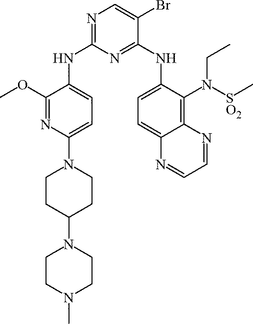 | I-364 | 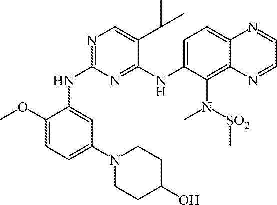 [SMILES: COc1ccc(N2CCC(O)CC2)cc1Nc1ncc(C(C)C)c(Nc2ccc3nccnc3c2N(C)S(C)(=O)=O)n1] |
| I-297 | 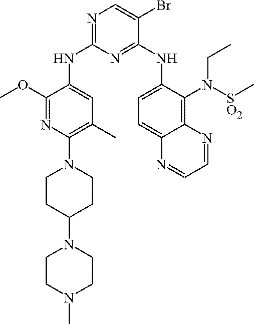 | I-365 | 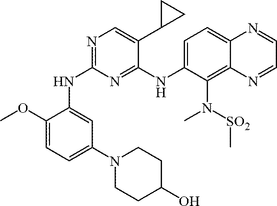 [SMILES: COc1ccc(N2CCC(O)CC2)cc1Nc1ncc(C2CC2)c(Nc2ccc3nccnc3c2N(C)S(C)(=O)=O)n1] |
| I-298 | 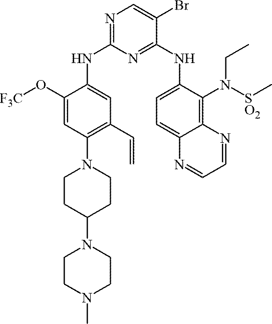 [SMILES: C=Cc1cc(Nc2ncc(Br)c(Nc3ccc4nccnc4c3N(CC)S(C)(=O)=O)n2)c(OC(F)(F)F)cc1N1CCC(N2CCN(C)CC2)CC1] | I-366 | 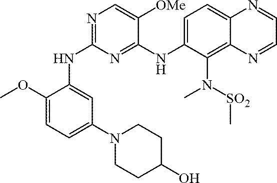 [SMILES: COc1ccc(N2CCC(O)CC2)cc1Nc1ncc(OC)c(Nc2ccc3nccnc3c2N(C)S(C)(=O)=O)n1] |
| I-299 | 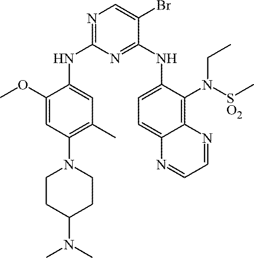 | I-367 | 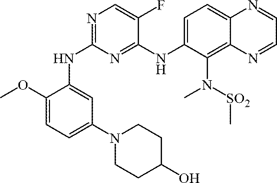 [SMILES: COc1ccc(N2CCC(O)CC2)cc1Nc1ncc(F)c(Nc2ccc3nccnc3c2N(C)S(C)(=O)=O)n1] |
| I-300 | 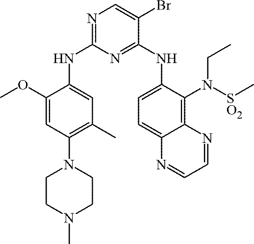 | I-368 | 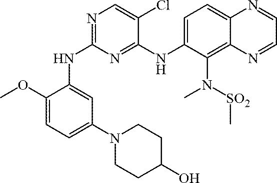 [SMILES: COc1ccc(N2CCC(O)CC2)cc1Nc1ncc(Cl)c(Nc2ccc3nccnc3c2N(C)S(C)(=O)=O)n1] |
| I-301 | 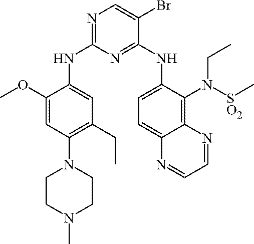 | I-369 | 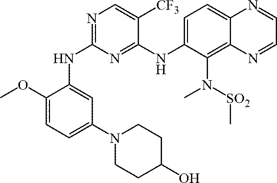 [SMILES: COc1ccc(N2CCC(O)CC2)cc1Nc1ncc(C(F)(F)F)c(Nc2ccc3nccnc3c2N(C)S(C)(=O)=O)n1] |
| I-302 | 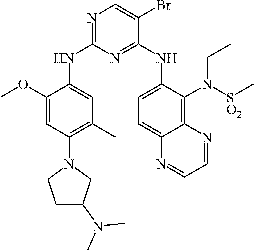 | I-370 | 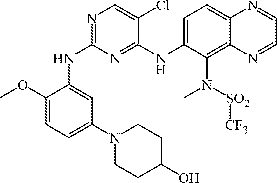 [SMILES: COc1ccc(N2CCC(O)CC2)cc1Nc1ncc(Cl)c(Nc2ccc3nccnc3c2N(C)S(=O)(=O)C(F)(F)F)n1] |
| I-303 | 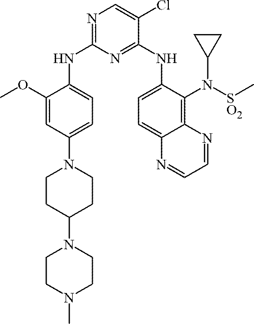 | I-371 | 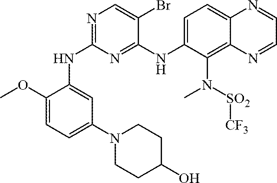 [SMILES: COc1ccc(N2CCC(O)CC2)cc1Nc1ncc(Br)c(Nc2ccc3nccnc3c2N(C)S(=O)(=O)C(F)(F)F)n1] |
| I-304 | 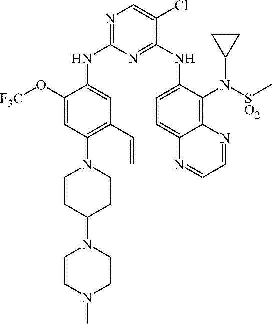 [SMILES: C=Cc1cc(Nc2ncc(Cl)c(Nc3ccc4nccnc4c3N(C3CC3)S(C)(=O)=O)n2)c(OC(F)(F)F)cc1N1CCC(N2CCN(C)CC2)CC1] | I-372 | 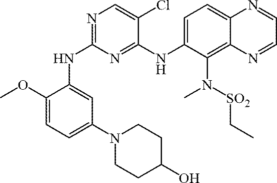 [SMILES: CCS(=O)(=O)N(C)c1c(Nc2nc(Nc3cc(N4CCC(O)CC4)ccc3OC)ncc2Cl)ccc2nccnc12] |
| I-305 | 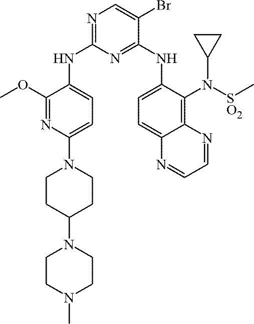 | I-373 | 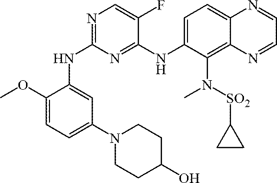 [SMILES: COc1ccc(N2CCC(O)CC2)cc1Nc1ncc(F)c(Nc2ccc3nccnc3c2N(C)S(=O)(=O)C2CC2)n1] |
| I-306 | 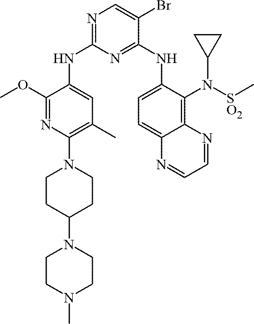 | I-374 | 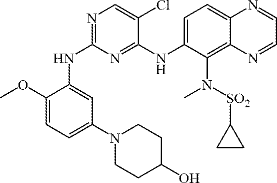 [SMILES: COc1ccc(N2CCC(O)CC2)cc1Nc1ncc(Cl)c(Nc2ccc3nccnc3c2N(C)S(=O)(=O)C2CC2)n1] |
| I-307 | 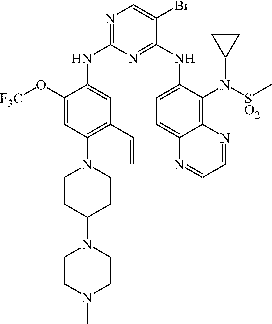 [SMILES: C=Cc1cc(Nc2ncc(Br)c(Nc3ccc4nccnc4c3N(C3CC3)S(C)(=O)=O)n2)c(OC(F)(F)F)cc1N1CCC(N2CCN(C)CC2)CC1] | I-375 | 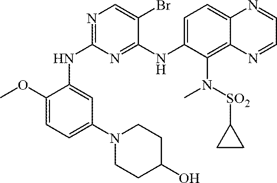 [SMILES: COc1ccc(N2CCC(O)CC2)cc1Nc1ncc(Br)c(Nc2ccc3nccnc3c2N(C)S(=O)(=O)C2CC2)n1] |
| I-308 | 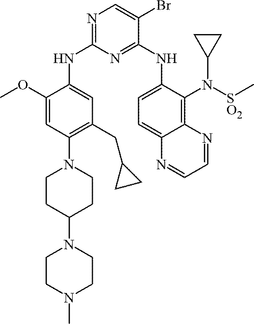 | I-376 | 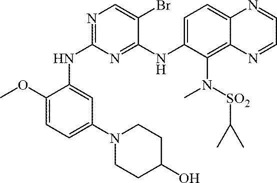 [SMILES: COc1ccc(N2CCC(O)CC2)cc1Nc1ncc(Br)c(Nc2ccc3nccnc3c2N(C)S(=O)(=O)C(C)C)n1] |
| I-309 | 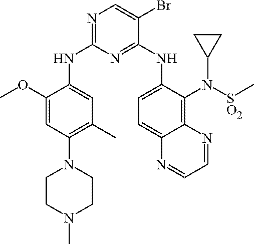 | I-377 | 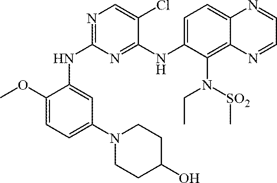 [SMILES: CCN(c1c(Nc2nc(Nc3cc(N4CCC(O)CC4)ccc3OC)ncc2Cl)ccc2nccnc12)S(C)(=O)=O] |
| I-310 | 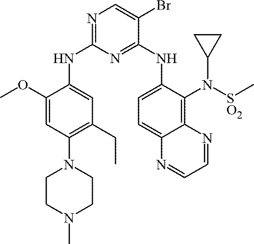 | I-378 | 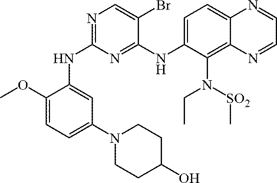 [SMILES: CCN(c1c(Nc2nc(Nc3cc(N4CCC(O)CC4)ccc3OC)ncc2Br)ccc2nccnc12)S(C)(=O)=O] |
| I-311 | 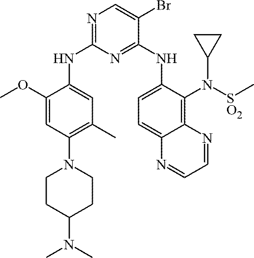 | I-379 | 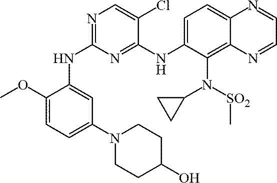 [SMILES: COc1ccc(N2CCC(O)CC2)cc1Nc1ncc(Cl)c(Nc2ccc3nccnc3c2N(C2CC2)S(C)(=O)=O)n1] |
| I-312 | 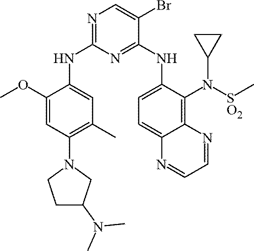 | I-380 | 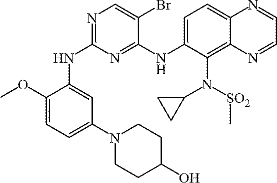 [SMILES: COc1ccc(N2CCC(O)CC2)cc1Nc1ncc(Br)c(Nc2ccc3nccnc3c2N(C2CC2)S(C)(=O)=O)n1] |
| I-313 | 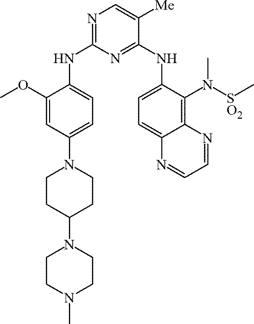 | I-381 | 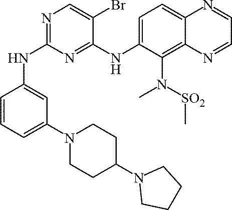 [SMILES: CN(c1c(Nc2nc(Nc3cccc(N4CCC(N5CCCC5)CC4)c3)ncc2Br)ccc2nccnc12)S(C)(=O)=O] |
| I-314 | 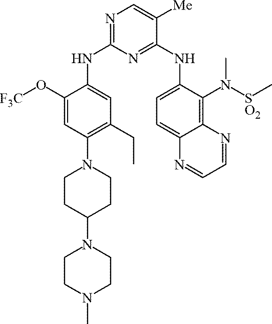 [SMILES: CCc1cc(Nc2ncc(C)c(Nc3ccc4nccnc4c3N(C)S(C)(=O)=O)n2)c(OC(F)(F)F)cc1N1CCC(N2CCN(C)CC2)CC1] | I-382 | 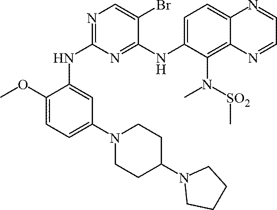 [SMILES: COc1ccc(N2CCC(N3CCCC3)CC2)cc1Nc1ncc(Br)c(Nc2ccc3nccnc3c2N(C)S(C)(=O)=O)n1] |
| I-315 | 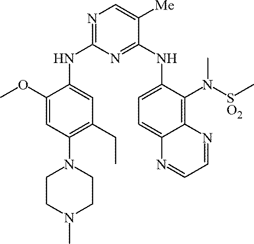 | I-383 | 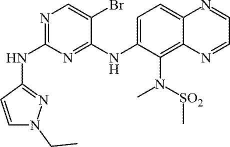 [SMILES: CCn1ccc(Nc2ncc(Br)c(Nc3ccc4nccnc4c3N(C)S(C)(=O)=O)n2)n1] |
| I-316 | 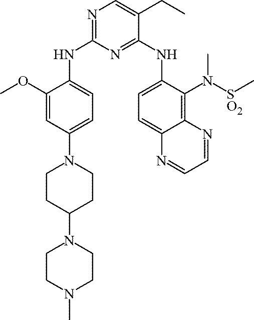 | I-384 | 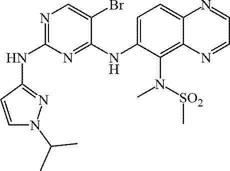 [SMILES: CC(C)n1ccc(Nc2ncc(Br)c(Nc3ccc4nccnc4c3N(C)S(C)(=O)=O)n2)n1] |
| I-317 | 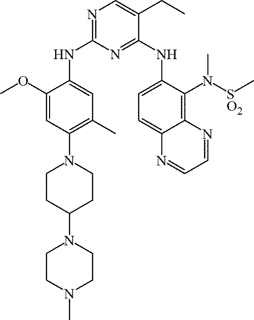 | I-385 | 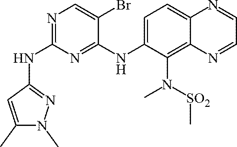 [SMILES: Cc1cc(Nc2ncc(Br)c(Nc3ccc4nccnc4c3N(C)S(C)(=O)=O)n2)nn1C] |
| I-318 | 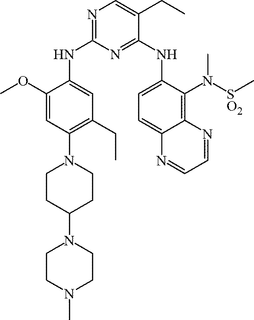 | I-386 | 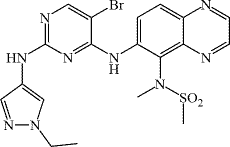 [SMILES: CCn1cc(Nc2ncc(Br)c(Nc3ccc4nccnc4c3N(C)S(C)(=O)=O)n2)cn1] |
| I-319 | 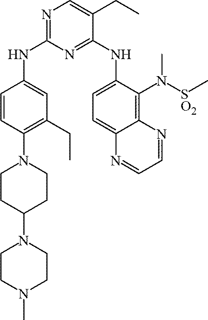 [SMILES: CCc1cc(Nc2ncc(CC)c(Nc3ccc4nccnc4c3N(C)S(C)(=O)=O)n2)ccc1N1CCC(N2CCN(C)CC2)CC1] | I-387 | 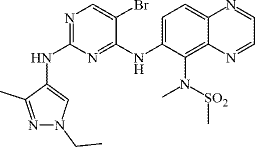 [SMILES: CCn1cc(Nc2ncc(Br)c(Nc3ccc4nccnc4c3N(C)S(C)(=O)=O)n2)c(C)n1] |
| I-320 | 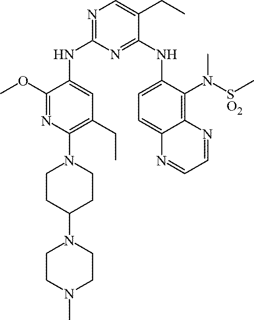 | 1-388 | 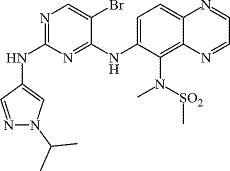 [SMILES: CC(C)n1cc(Nc2ncc(Br)c(Nc3ccc4nccnc4c3N(C)S(C)(=O)=O)n2)cn1] |
| I-321 | 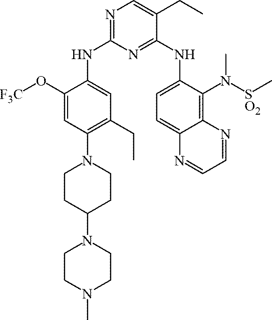 [SMILES: CCc1cc(Nc2ncc(CC)c(Nc3ccc4nccnc4c3N(C)S(C)(=O)=O)n2)c(OC(F)(F)F)cc1N1CCC(N2CCN(C)CC2)CC1] | I-389 | 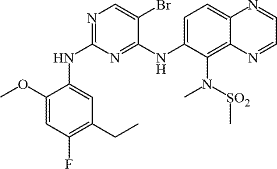 [SMILES: CCc1cc(Nc2ncc(Br)c(Nc3ccc4nccnc4c3N(C)S(C)(=O)=O)n2)c(OC)cc1F] |
| I-322 | 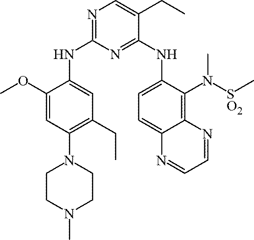 | I-390 | 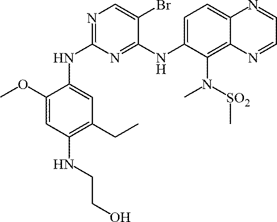 [SMILES: CCc1cc(Nc2ncc(Br)c(Nc3ccc4nccnc4c3N(C)S(C)(=O)=O)n2)c(OC)cc1NCCO] |
| I-323 | 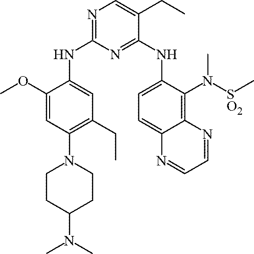 | I-391 | 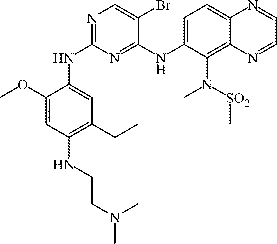 [SMILES: CCc1cc(Nc2ncc(Br)c(Nc3ccc4nccnc4c3N(C)S(C)(=O)=O)n2)c(OC)cc1NCCN(C)C] |
| I-324 | 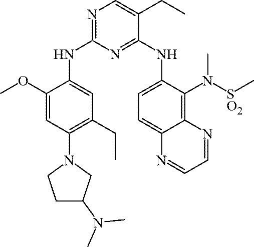 | I-392 | 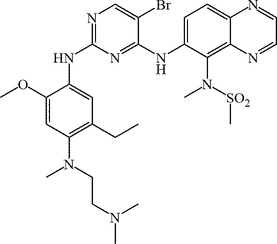 [SMILES: CCc1cc(Nc2ncc(Br)c(Nc3ccc4nccnc4c3N(C)S(C)(=O)=O)n2)c(OC)cc1N(C)CCN(C)C] |
| I-325 | 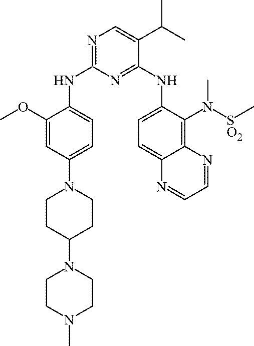 | I-393 | 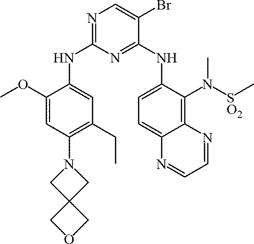 |
| I-326 | 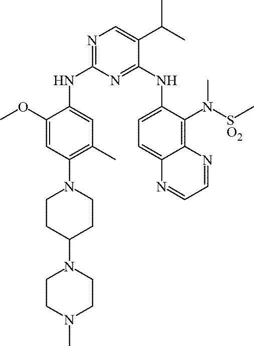 | I-394 | 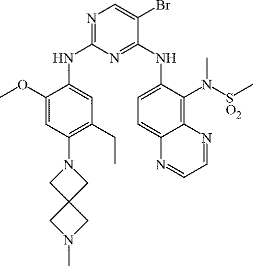 |
| I-327 | 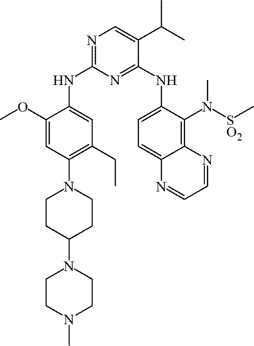 | I-395 | 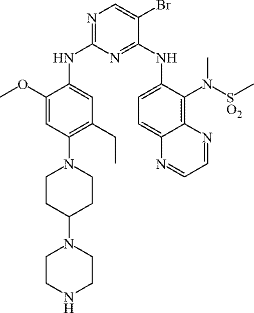 |
| I-328 | 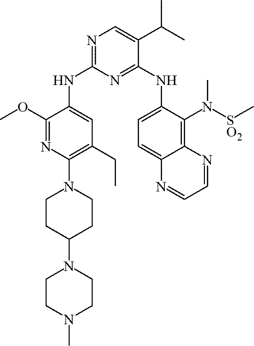 | I-396 | 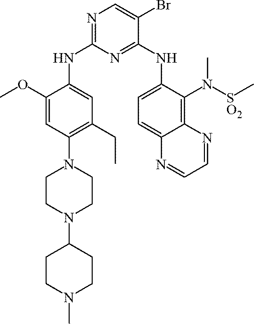 |
| I-329 | 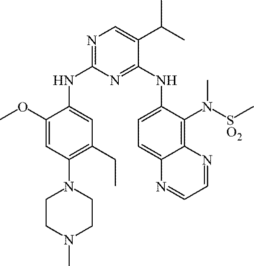 | I-397 | 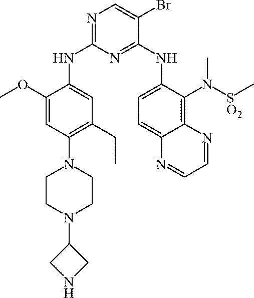 |
| I-330 | 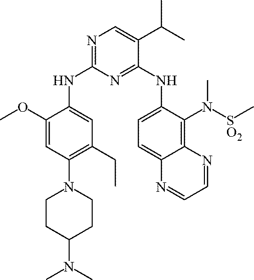 | I-398 | 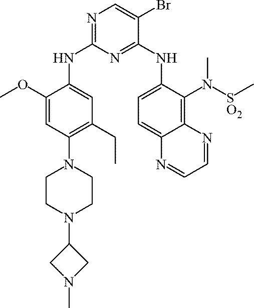 |
| I-331 | 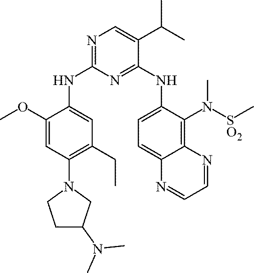 | I-399 | 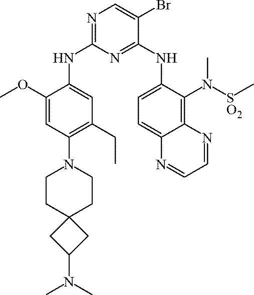 |
| I-332 | 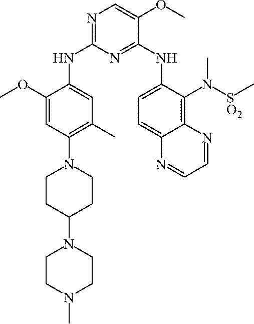 | I-400 | 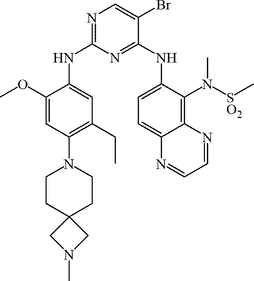 |
| I-333 | 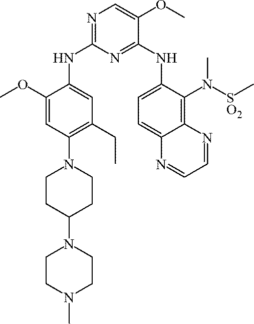 | I-401 | 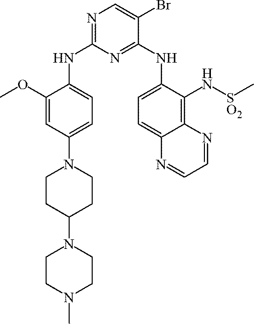 |
| I-334 | 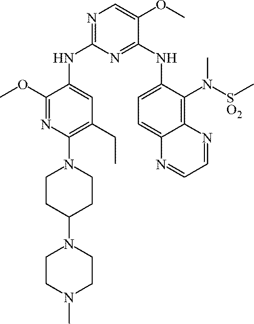 | I-402 | 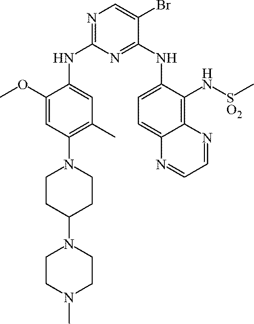 |
| I-335 | 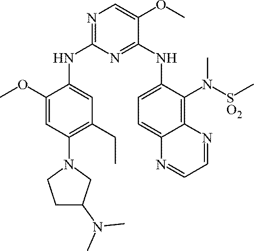 | I-403 | 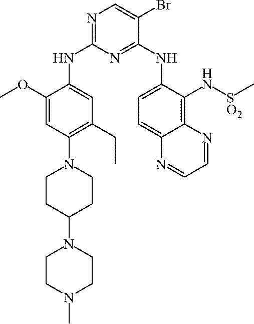 |
| I-336 | 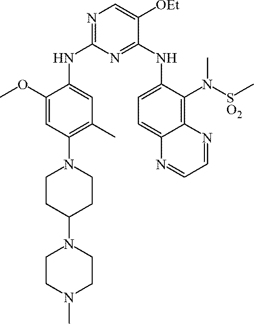 | I-404 | 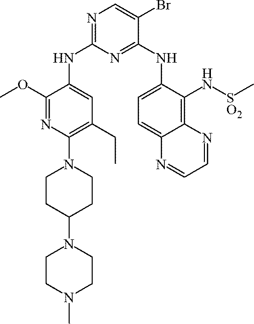 |
| I-337 | 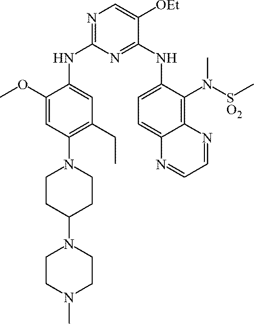 | I-405 | 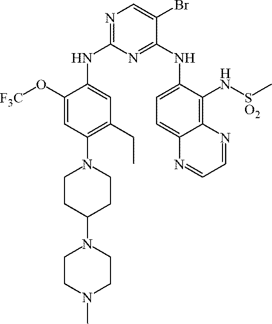 [SMILES: CCc1cc(Nc2ncc(Br)c(Nc3ccc4nccnc4c3NS(C)(=O)=O)n2)c(OC(F)(F)F)cc1N1CCC(N2CCN(C)CC2)CC1] |
| I-338 | 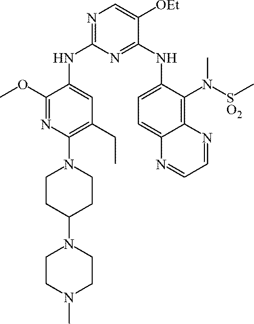 | I-406 | 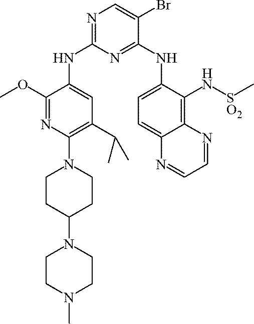 |
| I-339 | 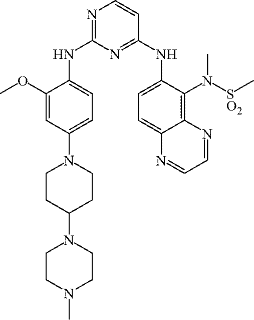 | I-407 | 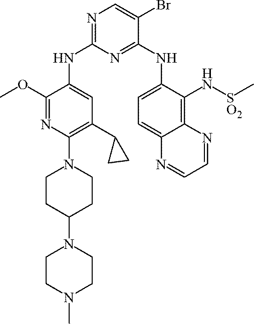 |
| I-340 | 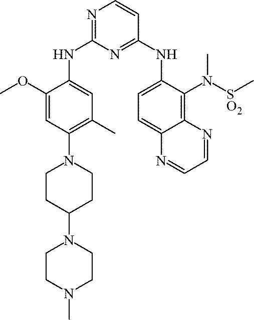 | I-408 | 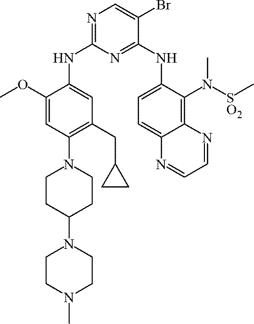 |
| I-341 | 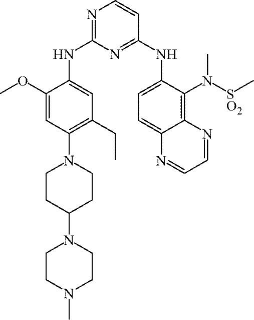 | I-409 | 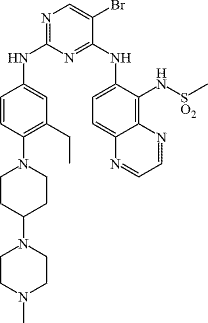 [SMILES: CCc1cc(Nc2ncc(Br)c(Nc3ccc4nccnc4c3NS(C)(=O)=O)n2)ccc1N1CCC(N2CCN(C)CC2)CC1] |
| I-342 | 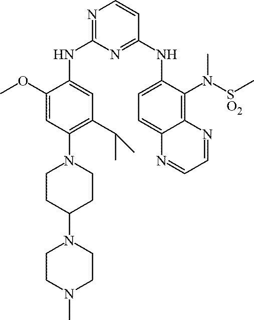 | I-410 | 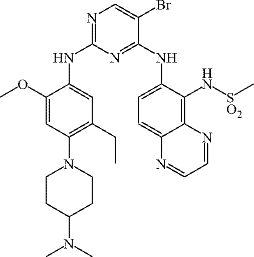 |
| I-343 | 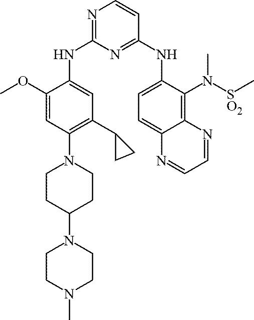 | I-411 | 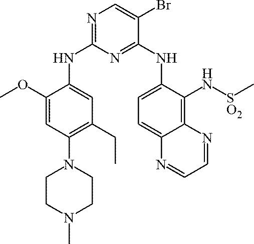 |
| I-344 | 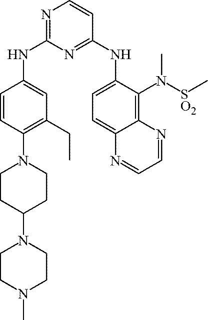 [SMILES: CCc1cc(Nc2nccc(Nc3ccc4nccnc4c3N(C)S(C)(=O)=O)n2)ccc1N1CCC(N2CCN(C)CC2)CC1] | I-412 | 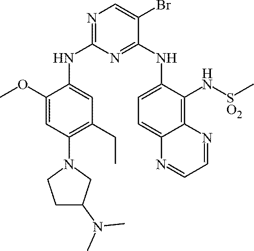 |
| I-345 | 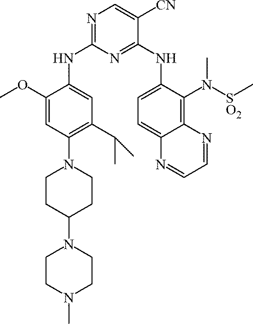 | I-413 | 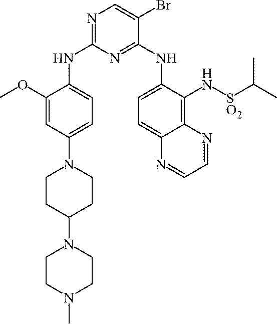 [SMILES: COc1cc(N2CCC(N3CCN(C)CC3)CC2)ccc1Nc1ncc(Br)c(Nc2ccc3nccnc3c2NS(=O)(=O)C(C)C)n1] |
| I-346 | 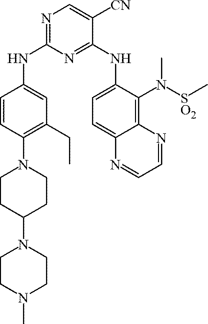 [SMILES: CCc1cc(Nc2ncc(C#N)c(Nc3ccc4nccnc4c3N(C)S(C)(=O)=O)n2)ccc1N1CCC(N2CCN(C)CC2)CC1] | I-414 | 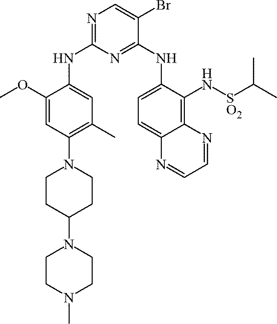 [SMILES: COc1cc(N2CCC(N3CCN(C)CC3)CC2)c(C)cc1Nc1ncc(Br)c(Nc2ccc3nccnc3c2NS(=O)(=O)C(C)C)n1] |
| I-347 | 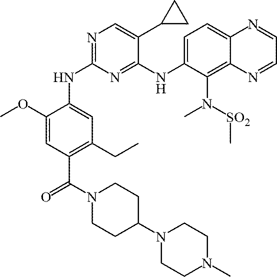 [SMILES: CCc1cc(Nc2ncc(C3CC3)c(Nc3ccc4nccnc4c3N(C)S(C)(=O)=O)n2)c(OC)cc1C(=O)N1CCC(N2CCN(C)CC2)CC1] | I-415 | 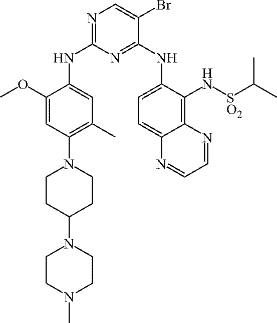 [SMILES: COc1cc(N2CCC(N3CCN(C)CC3)CC2)c(C)cc1Nc1ncc(Br)c(Nc2ccc3nccnc3c2NS(=O)(=O)C(C)C)n1] |
| I-348 | 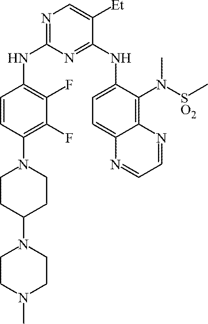 [SMILES: CCc1cnc(Nc2ccc(N3CCC(N4CCN(C)CC4)CC3)c(F)c2F)nc1Nc1ccc2nccnc2c1N(C)S(C)(=O)=O] | II-1-45 | 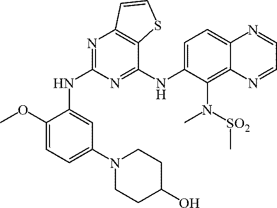 [SMILES: COc1ccc(N2CCC(O)CC2)cc1Nc1nc(Nc2ccc3nccnc3c2N(C)S(C)(=O)=O)c2sccc2n1] |
| I-349 | 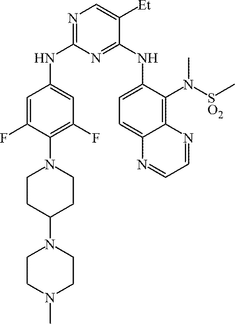 [SMILES: CCc1cnc(Nc2cc(F)c(N3CCC(N4CCN(C)CC4)CC3)c(F)c2)nc1Nc1ccc2nccnc2c1N(C)S(C)(=O)=O] | II-1-46 | 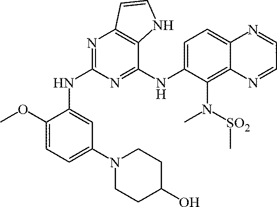 [SMILES: COc1ccc(N2CCC(O)CC2)cc1Nc1nc(Nc2ccc3nccnc3c2N(C)S(C)(=O)=O)c2[nH]ccc2n1] |
| I-350 | 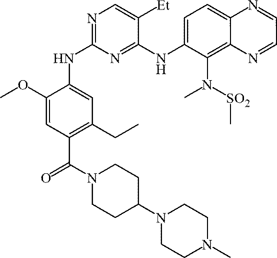 [SMILES: CCc1cc(Nc2ncc(CC)c(Nc3ccc4nccnc4c3N(C)S(C)(=O)=O)n2)c(OC)cc1C(=O)N1CCC(N2CCN(C)CC2)CC1] | II-2-23 | 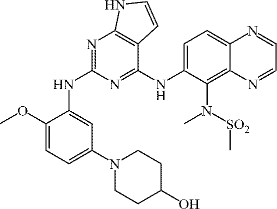 [SMILES: COc1ccc(N2CCC(O)CC2)cc1Nc1nc(Nc2ccc3nccnc3c2N(C)S(C)(=O)=O)c2cc[nH]c2n1] |
| I-351 | 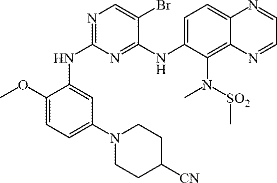 [SMILES: COc1ccc(N2CCC(C#N)CC2)cc1Nc1ncc(Br)c(Nc2ccc3nccnc3c2N(C)S(C)(=O)=O)n1] | | |

or stereoisomers, prodrugs, pharmaceutically acceptable salts or pharmaceutically acceptable solvates of the above compounds.

5. A method of preparing the compound according to claim 1, comprising the following steps: 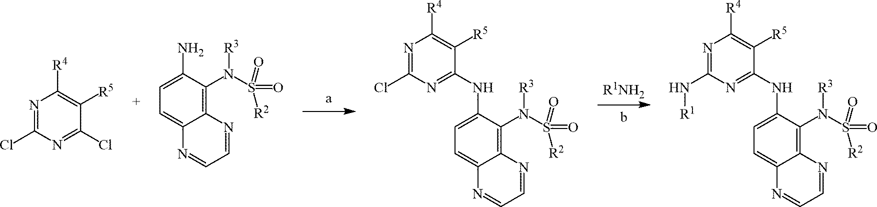
wherein, the definitions of R
¹, R², R³, R⁴ and R⁵ are as described in claim 1, reaction conditions are as follows:
(a) a nucleophilic substitution reaction under acidic or alkaline condition;
(b) a coupling reaction catalyzed by metal palladium, or a nucleophilic substitution reaction under acidic condition; wherein:
the metal palladium catalyst is selected from palladium acetate, tetrakis(triphenylphosphine) palladium, bistriphenylphosphine palladium dichloride, [1,1′-bis(diphenylphosphino)ferrocene] palladium dichloride or tris(dibenzylideneacetone) dipalladium;
the alkaline condition refers to a condition in which any of the following substances exists: triethylamine, diisopropylethylamine, pyridine, sodium bicarbonate, sodium carbonate, potassium carbonate, cesium carbonate, lithium hydroxide, sodium hydroxide, potassium hydroxide, sodium hydride, or potassium hydride;
the acidic condition refers to a condition in which any of the following substances exists: acetic acid, trifluoroacetic acid, hydrochloric acid, methanesulfonic acid, p-toluene sulfonic acid, or camphorsulfonic acid.

6. A pharmaceutical composition comprising the compound according to claim 1 or a stereoisomer, a prodrug, a pharmaceutically acceptable salt or a pharmaceutically acceptable solvate thereof and optionally a pharmaceutically acceptable excipient,
preferably, the pharmaceutical composition further comprises a EGFR monoclonal antibody;
preferably, the EGFR monoclonal antibody is Cetuximab or biosimilars thereof.

7. Use of the compound according to claim 1, or a stereoisomer thereof, a prodrug thereof, a pharmaceutically acceptable salt thereof or a pharmaceutically acceptable solvate thereof, claim in the manufacture of a medicament for preventing and/or treating EGFR kinase-mediated cancers and other diseases, especially in the manufacture of a medicament for preventing and/or treating lung cancer (preferably non-small cell lung cancer), particularly in the manufacture of a medicament for preventing and/or treating mutation-type lung cancer (preferably non-small cell lung cancer) with 19Del, L858R, T790M, or C797S mutation or a combination thereof of EGFR kinase, and most preferably in the manufacture of a medicament for preventing and/or treating lung cancer (preferably non-small cell lung cancer) with 19Del single mutation, L858R single mutation, 19Del/T790M dual mutation, L858R/T790M dual mutation, 19Del/T790M/C797S triple mutation or L858R/T790M/C797S triple mutation of EGFR kinase.

8. A method of preventing and/or treating EGFR kinase-mediated cancers and other diseases, comprising administering a preventively effective amount and/or a therapeutically effective amount of the compound according to any one of claim 1, or a stereoisomer thereof, a prodrug thereof, a pharmaceutically acceptable salt thereof or a pharmaceutically acceptable solvate thereof to a subject in need,
preferably, the method is for preventing and/or treating lung cancer (preferably non-small cell lung cancer);
more preferably, the method is for preventing and/or treating mutation-type lung cancer (preferably non-small cell lung cancer) with 19Del, L858R, T790M, or C797S mutation or a combination thereof of EGFR kinase;
most preferably, the method is for preventing and/or treating lung cancer (preferably non-small cell lung cancer) with 19Del single mutation, L858R single mutation, 19Del/T790M dual mutation, L858R/T790M dual mutation, 19Del/T790M/C797S triple mutation or L858R/T790M/C797S triple mutation of EGFR kinase.

9. The compound according to claim 1 or a stereoisomer thereof, a prodrug thereof, a pharmaceutically acceptable salt thereof or a pharmaceutically acceptable solvate thereof, for use in preventing and/or treating EGFR kinase-mediated cancers and other diseases, preferably for use in preventing and/or treating lung cancer (preferably non-small cell lung cancer), more preferably for use in preventing and/or treating mutation-type lung cancer (preferably non-small cell lung cancer) with 19Del, L858R, T790M, or C797S mutation or a combination thereof of EGFR kinase, and most preferably for use in preventing and/or treating lung cancer (preferably non-small cell lung cancer) with 19Del single mutation, L858R single mutation, 19Del/T790M dual mutation, L858R/T790M dual mutation, 19Del/T790M/C797S triple mutation or L858R/T790M/C797S triple mutation of EGFR kinase.
